(19) United States
(12) Patent Application Publication
(10) Pub. No.: US 2025/0325665 A1
(43) Pub. Date: Oct. 23, 2025

(54) FUSION CONSTRUCTS AND METHODS OF USING THEREOF

(71) Applicant: PRECIGEN, INC., Germantown, MD (US)

(72) Inventors: Helen SABZEVARI, Germantown, MD (US); Simon METENOU, Germantown, MD (US); Chang Hung CHEN, Germantown, MD (US); Rutul SHAH, Germantown, MD (US)

(73) Assignee: PRECIGEN, INC., Germantown, MD (US)

(21) Appl. No.: 18/966,676

(22) Filed: Dec. 3, 2024

Related U.S. Application Data
(62) Division of application No. 17/305,577, filed on Jul. 9, 2021, now Pat. No. 12,194,094.
(60) Provisional application No. 63/050,393, filed on Jul. 10, 2020.
(60) Provisional application No. 63/173,902, filed on Apr. 12, 2021.

(51) Int. Cl. A61K 39/395; A61K 38/17; A61K 38/20; A61K 39/00; A61K 40/11; A61K 40/31; A61K 40/42; C07K 14/71; C07K 16/28; C07K 16/30

(52) U.S. Cl. CPC A61K 39/39558; A61K 38/179; A61K 38/1793; A61K 38/2086; A61K 39/001102; A61K 39/001112; A61K 39/001129; A61K 39/00117; A61K 39/001195; A61K 40/11; A61K 40/31; A61K 40/4202; A61K 40/421; A61K 40/4211; A61K 40/4215; A61K 40/4224; A61K 40/4229; A61K 40/4257; A61K 40/4276; C07K 14/71; C07K 16/2818; C07K 16/2857; C07K 16/2863; C07K 16/2866; C07K 16/2875; C07K 16/2878; C07K 16/2884; C07K 16/2887; C07K 16/2896; C07K 16/30; C07K 16/3069; C07K 16/3092; A61K 2239/50; A61K 2239/54; A61K 2239/55; A61K 2239/59

(57) ABSTRACT

A fusion protein comprising: a first component comprising an antibody, or a fragment or variant thereof; and a second component comprising a cytokine trap or an adenosine deaminase or a fragment or variant thereof. In certain embodiments, the antibody is an anti-PD-1 antibody. In certain embodiments, the antibody binds to a tumor antigen, for example a MUC16 or MUC1 antigen. In certain embodiments, the cytokine trap is a TGF-β trap. A polynucleotide encoding such a fusion protein and a vector comprising such a polynucleotide. A composition comprising the fusion protein. A method of using the composition, including in the treatment of cancer.

### CROSS-REFERENCE TO RELATED APPLICATIONS

The present application is a division of U.S. application Ser. No. 17/305,577, filed Jul. 9, 2021, which in turn claims the benefit of U.S. Provisional Patent Application Nos. 63/050,393, filed Jul. 10, 2020, and 63/173,902, filed Apr. 12, 2021. Each of these applications is hereby incorporated by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SEQUENCE LISTING

The instant application contains a Sequence Listing that has been submitted electronically in XML format and is hereby incorporated by reference in its entirety. Said XML copy, created on Nov. 26, 2024, is named 17305577DIVSL.xml and is 745,458 bytes in size.

### BACKGROUND OF THE DISCLOSURE

Recently, monoclonal antibody-based cancer immunotherapy based on the interruption of suppressive signals that are delivered to the adaptive immune system has shown promise in the clinic. With the FDA approval of CTLA-4 antibody inhibitor (e.g., ipilimumab) and PD-1 inhibitors (e.g., pembrolizumab, nivolumab), more treatment options are now available to treat solid tumors including lung cancer, renal cell cancer, and ovarian cancer. However, in the majority of indications where PD-1/PD-L1 and TGF-β are co-expressed (e.g., ovarian, gastric and colorectal) little to no response to immune checkpoint inhibitors has been observed. Accordingly, there is a continuing need in the art to obtain safer and more effective treatments for cancer.

### SUMMARY OF THE DISCLOSURE

Provided herein is a fusion protein comprising: (a) a first component that is an antibody, or a fragment or variant thereof; and (b) a second component that is: (i) a cytokine trap, or fragment or variant thereof; or (ii) an adenosine deaminase (ADA), or fragment or variant.

In certain embodiments, first component is an immunoglobulin G (lgG) antibody, for example an IgG1, IgG2, IgG3, or IgG4 antibody. In certain embodiments, the antibody is an IgG1 or IgG4 antibody.

In certain embodiments, the antibody fragment of the first component is a Fab, (Fab)₂, (Fab′)₂, Fv, (Fv)₂, or scFv.

In certain embodiments, the antibody, or fragment or variant thereof, of the first component comprises a variable region of a heavy chain (V_{H}) and a variable region of a light chain (V_{L}).

In certain embodiments, the antibody, or fragment or variant thereof, of the first component comprises a fragment crystallizable region (Fc), for example an Fc1, Fc2, Fc3, or Fc4 region, or a fragment or variant thereof. In certain embodiments, the antibody, or fragment or variant thereof, comprises an Fc1 region. In certain embodiments, the Fc comprises at least one mutation.

In certain embodiments, the antibody, or fragment or variant thereof, of the first component comprises a scFv and an Fc.

In certain embodiments, the antibody, or fragment or variant thereof, of the first component binds to a tumor antigen expressed on the surface of a tumor cell. Examples of such a tumor antigen include CD19, BCMA, CD23, BAFF-R, GPRC5D, CD44, CAIX, CD5, CD30, CD70, CD44v6, CD44v7, CD44v8, CD174, CD28, CD128, CD138, CS1, CLL-1, L1-CAM, FAP, ROR1, CEA, EGP-2, EGP-40, HER2, HER3, Folate-binding Protein, GD2, GD3, IL-13R-a2, IL-11Ra, EphA2, CSPG4, KDR, EDB-F, mesothelin, CD22, EGFR, Folate receptor α, MUC-1, MUC-4, MUC-16, MAGE-A1, h5T4, PSMA, PSCA, GPC3, c-met, TAG-72, EGFR, CD20, EGFRVIII, CD123, and VEGF-R2. In certain embodiments, the tumor antigen is a mucin, for example a MUCI, MUC2, MUC3A, MUC3B, MUC4, MUC5AC, MUC5B, MUC6, MUC7, MUC8, MUC12, MUC13, MUC15, MUC16, MUC17, MUC19, or MUC20 antigen.

In certain embodiments, the antibody, or fragment or variant thereof, of the first component binds to MUC16. For example, the antibody, or fragment or variant thereof, may comprise a V_{L} region comprising a sequence that is at least 80% identical to any one of SEQ ID NOs: 417-428 and/or a V_{H} region comprising a sequence that is at least 80% identical to any one of SEQ ID NOs: 390-403. In certain embodiments, the antibody, or fragment or variant thereof, comprises a V_{H} region comprising a sequence that is at least 80% identical to SEQ ID NO: 399 and/or a V_{L} region comprising a sequence that is at least 80% identical to SEQ ID NO: 426. In certain embodiments, the antibody, or fragment or variant thereof, comprises a heavy chain sequence that is at least 80% identical to SEQ ID NO: 441 and/or a light chain sequence that is at least 80% identical to SEQ ID NO: 443.

In certain embodiments, the antibody, or fragment or variant thereof, of the first component binds to MUCI. For example, the antibody, or fragment or variant thereof, may comprise a V_{L} region comprising a sequence that is at least 80% identical to any one of SEQ ID NOs: 449-453, and/or a V_{H} region comprising a sequence that is at least 80% identical to any one of SEQ ID NOs: 444-448. In certain embodiments, the antibody, or fragment or variant thereof, comprises a V_{H} region comprising a sequence that is at least 80% identical to SEQ ID NO: 444. In certain embodiments, the antibody, or fragment or variant thereof, comprises a V_{H} region comprising a sequence that is at least 80% identical to SEQ ID NO: 444 and a V_{L} region comprising a sequence that is at least 0% identical to any one of SEQ ID NOs: 449-453. In certain embodiments, the antibody, or fragment or variant thereof, comprises a heavy chain sequence that is at least 80% identical to SEQ ID NO: 466 and/or a light chain sequence that is at least 80% identical to SEQ ID NO: 467.

In certain embodiments, the antibody, or fragment or variant thereof, of the first component binds to programmed cell death protein-1 (PD-1). Such an antibody will be referred to herein as an “anti-PD-1 antibody.”

In certain embodiments, the antibody of the first component is an IgG4 antibody comprising a sequence having at least 80% sequence identity to SEQ ID NO: 146 or 292 and having a mutation at position 108 thereof. For example, the antibody may be an IgG4 antibody comprising a sequence having at least 80% sequence identity to SEQ ID NO: 146 or 292 and having a S108P mutation (i.e., amino acid proline in place of serine at position 108).

In certain embodiments, the antibody, or fragment or variant thereof, of the first component comprises a V_{H} or a V_{L} that is connected to the second component by a linker.

In certain embodiments, the antibody, or fragment or variant thereof, of the first component comprises a V_{H} and a V_{L}, and the V_{H} and V_{L} are connected to each other by a second linker, for example one comprising a sequence of any one of SEQ ID NOs: 17-34.

In certain embodiments, the anti-PD-1 antibody, or fragment or variant thereof, comprises a V_{H} comprising a sequence that is at least 80% identical to any one of SEQ ID NOs: 1-7, 149-164, 333-337, and 384 and/or a V_{L} comprising a sequence that is at least 80% identical to any one of SEQ ID NOs: 8-13, 148, 338-343, and 385. For example, the antibody, or fragment or variant thereof, may comprise: a V_{H} comprising a sequence that is at least 90% identical to SEQ ID NO: 6 and a V_{L} comprising a sequence that is at least 90% identical to SEQ ID NO: 12; a V_{H} comprising a sequence that is at least 90% identical to SEQ ID NO: 7 and a V_{L} comprising a sequence that is at least 90% identical to SEQ ID NO: 13; a V_{H} comprising a sequence that is at least 90% identical to SEQ ID NO: 333 and a V_{L} comprising a sequence that is at least 90% identical to SEQ ID NO: 338; a V_{H} comprising a sequence that is at least 90% identical to SEQ ID NO: 334 and a V_{L} comprising a sequence that is at least 90% identical to SEQ ID NO: 339; a V_{H} comprising a sequence that is at least 90% identical to SEQ ID NO: 335 and a V_{L} comprising a sequence that is at least 90% identical to SEQ ID NO: 340; a V_{H} comprising a sequence that is at least 90% identical to SEQ ID NO: 336 and a V_{L} comprising a sequence that is at least 90% identical to SEQ ID NO: 341; a V_{H} comprising a sequence that is at least 90% identical to SEQ ID NO: 337 and a V_{L} comprising a sequence that is at least 90% identical to SEQ ID NO: 342; a V_{H} comprising a sequence that is at least 90% identical to SEQ ID NO: 335 and a V_{L} comprising a sequence that is at least 90% identical to SEQ ID NO: 343; a V_{H} comprising a sequence that is at least 90% identical to SEQ ID NO: 384 and a V_{L}, comprising a sequence that is at least 90% identical to SEQ ID NO: 385; or a V_{H} comprising a sequence that is at least 90% identical to SEQ ID NO: 386 and a V_{L} comprising a sequence that is at least 90% identical to SEQ ID NO: 387.

In certain embodiments, the anti-PD-1 antibody, or fragment or variant thereof, is: AM-0001; AMP-224: balstilimab; budigalimab; BI 754091; camrelizumab: cemiplimab; cetrelimab; dostarlimab: JTX-4014; MEDI-0680; MGA012; nivolumab; pembrolizumab; pidilizumab; prolgolimab; sasanlimab; sintilimab; spartalizumab; STI-1110; tislelizumab; toripalimab; or zimberelimab; or a fragment or variant thereof. For example, the anti-PD-1 antibody, or fragment or variant thereof, is: pembrolizumab; nivolumab; zimberelimab; or cetrelimab; or a fragment or variant thereof. In certain embodiments, the anti-PD-1 antibody, or fragment or variant thereof, of the first component is cetrelimab, or a fragment or variant thereof.

In certain embodiments, the anti-PD-1 antibody, or fragment or variant thereof, comprises a sequence that is at least 90% identical to SEQ ID NO: 15 and a sequence that is at least 90% identical to SEQ ID NO: 16 or 143. In certain embodiments, the anti-PD-1 antibody, or fragment or variant thereof, comprises a sequence that is at least 80% identical to SEQ ID NO: 296 and a sequence that is at least 80% identical to any one of SEQ ID NOs: 144, 145, and 295. In certain embodiments, the anti-PD-1 antibody, or fragment or variant thereof, comprises a sequence that is at least 80% identical to SEQ ID NO: 13 or 15 and a sequence that is at least 80% identical to SEQ ID NO: 294 or 295.

In certain embodiments, the second component of the fusion protein is a cytokine trap, for example a TGF-β cytokine trap. The TGF-β cytokine trap may, for example, comprise: a transforming growth factor receptor (TGFβR) or a fragment or variant thereof; an anti-TGF-β antibody or an antigen binding fragment or variant thereof; a TGF-β inhibitory peptide or a fragment or variant thereof; and/or a TGF-β antagonistic peptide or a fragment or variant thereof.

In certain embodiments, the TGF-β cytokine trap comprises a transforming growth factor beta receptor II (TGFβRII) or a fragment or variant thereof.

In certain embodiments, the TGF-β cytokine trap comprises a TGFβR extracellular domain or a fragment or variant thereof. In certain embodiments, the TGF-β cytokine trap comprises a TGFβRII extracellular domain, or a fragment or variant thereof, for example comprising a sequence that is at least 80% identical to any one of SEQ ID NOs: 14, 141, and 142. In certain embodiments, the TGFβRII extracellular domain, or a fragment or variant thereof, binds TGF-β1 and/or TGF-β3. In certain embodiments, the TGFβRII extracellular domain, or a fragment or variant thereof, binds TGF-1 and TGF-β3 but does not bind TGF-β2 or binds TGF-β2 at a lower affinity than it does TGF-β1 and TGF-β3.

In certain embodiments, the TGF-β cytokine trap comprises a sequence that is at least 80% identical to any one of SEQ ID NOs: 14, 141, and 142.

In certain embodiments, the TGF-β cytokine trap comprises an anti-TGF-β antibody or an antigen binding fragment or variant thereof. The antibody, or antigen binding fragment or variant thereof, may for example comprise a variable region of a heavy chain (V_{H}) and a variable region of a light chain (V_{L}). In certain embodiments, the antibody, or antigen binding fragment or variant thereof, may comprise a V_{H} comprising a sequence that is at least 80% identical to any one of SEQ ID NOs: 166, 168, 169, 171, 173, and 175 and/or a V_{L} comprising a sequence that is at least 80% identical to any one of SEQ ID NOs: 165, 167, 170, 172, 174, 176, and 178.

In certain embodiments, the TGF-β cytokine trap comprises a TGF-β inhibitory peptide or a fragment or variant thereof, for example comprising a sequence that is at least 80% identical to any one of SEQ ID NOs: 468-507 and 263-267. In certain embodiments, the TGF-β cytokine trap comprises two or more TGF-β inhibitory peptides, or fragments or variants thereof. The two or more TGF-β inhibitory peptides, or fragments or variants thereof, may be connected by linker(s).

In certain embodiments, the TGF-β cytokine trap comprises a TGF-β antagonistic peptide or a fragment or variant thereof. In certain embodiments, the TGF-β cytokine trap comprising two or more TGF-β antagonistic peptides, or a fragments or variants thereof. The two or more TGF-β antagonistic peptides, or fragments or variants thereof, may be connected by linker(s).

In certain embodiments, the second component of the fusion protein is an ADA or a fragment or variant thereof.

In certain embodiments, the ADA, of fragment or variant thereof, is adenosine deaminase 2 (ADA2) or a fragment or variant thereof, for example one comprising a sequence that is at least 80% identical to any one of SEQ ID NOs: 273-279 and 284. In certain embodiments, the ADA2, or fragment or variant thereof, comprises at least one amino acid substitution or deletion.

In certain embodiments, the first and second components are connected by a linker. The linker may, for example, comprise: (G4S)ₙ, wherein n is 2, 3, 4, 5, or 6 (SEQ ID NO: 555); (Gly)ₙ, wherein n is 6, 7, or 8 (SEQ ID NO: 33); (EAAAK)ₙ, wherein n is 1, 2, 3, 4, 5, or 6 (SEQ ID NO: 34); A (EAAAK)₄ALEA (EAAAK)₄A (SEQ ID NO: 31); and/or a sequence of any one of SEQ ID NOs: 17-34.

Also provided herein a polynucleotide encoding the fusion protein as described above. Further provided is an expression vector comprising such a polynucleotide operably linked to a promoter, for example, a constitutive promoter, a tissue specific promoter, or an inducible promoter. The promoter may be a small molecule ligand-inducible two polypeptide ecdysone receptor-based gene switch promoter. The vector may be an adenoviral vector.

Further provided herein is a pharmaceutical composition comprising: the fusion protein as described above or a polynucleotide encoding the same; and a pharmaceutically-acceptable excipient. In certain embodiments, the polynucleotide may be contained in an expression vector.

Also provided is a method of treating cancer, the method comprising contacting a cell with the fusion protein as described above or a polynucleotide encoding the same. In embodiments wherein the cell is contacted with a polynucleotide, the polynucleotide may be contained in an expression vector and the cell contacted with the vector. In certain embodiments the cell is a cancer cell. In certain embodiments, the cell is a mammalian cell. In certain embodiments, the mammalian cell is an immune cell. In certain embodiments, the immune cell is a T cell.

Further provided is a method of treating cancer in a subject in need of such treatment, the method comprising administering the aforementioned composition comprising the fusion protein to a subject. In certain embodiments, the cancer is a refractory cancer. In certain embodiments, the subject is non-responsive to a treatment with an anti-PD-1 antibody or a CTLA-4 antibody. In certain embodiments, the method further comprises administering one or more additional anti-cancer agent, for example a PD-1 inhibitor (e.g., an anti-PD-1 antibody, or a fragment or variant thereof), PD-L1 inhibitor, and/or a CTLA-4 inhibitor (e.g., an anti-CTLA-4 antibody or a fragment or variant thereof). In certain embodiments, the method further comprises administering a cytokine, for example a fusion protein comprising IL-15 and IL-15Ra. In certain embodiments, the subject is a mammalian subject, for example a human. In certain embodiments, the cancer is mesothelioma, glioblastoma, endometrial cancer, colorectal cancer, gastric cancer, cervical cancer, ovarian cancer, pancreatic cancer, prostate cancer, breast cancer, stomach cancer, bladder cancer, liver cancer, Hodgkin's lymphoma, lung cancer, skin cancer, renal cancer, head and neck cancer, melanoma, bronchus cancer, urinary tract cancer, anal cancer, brain cancer, esophageal cancer, cervical cancer, uterine cancer, cancer of the oral cavity or pharynx, kidney cancer, testicular cancer, biliary tract cancer, small bowel cancer, appendix cancer, salivary gland cancer, thyroid gland cancer, adrenal gland cancer, osteosarcoma, chondrosarcoma, or a cancer of a hematological tissue. In certain embodiments, the cancer is cutaneous squamous-cell carcinoma, melanoma or basal cell cancer. In certain embodiments, the cancer is non-small cell lung cancer (NSLC) or small cell lung cancer (SCLC). In certain embodiments, the cancer is triple negative breast cancer (TNBC).

In certain embodiments, the method further comprises administering an effective amount of T cells engineered to express an exogenous receptor. In certain embodiments, the exogenous receptor is a chimeric antigen receptor, for example one that comprises an antigen binding domain that binds to an epitope on CD19, BCMA, CD23, BAFF-R, GPRC5D, CD44, CAIX, CD5, CD30, CD70, CD44v6, CD44v7, CD44v8, CD174, CD28, CD128, CD138, CS1, CLL-1, L1-CAM, FAP, ROR1, CEA, EGP-2, EGP-40, HER2, HER3, Folate-binding Protein, GD2, GD3, IL-13R-a2, IL-11Ra, EphA2, CSPG4, KDR, EDB-F, mesothelin, CD22, EGFR, Folate receptor α, MUC-1, MUC-4, MUC-16, MAGE-A1, h5T4, PSMA, PSCA, GPC3, c-mct, TAG-72, EGFR, CD20, EGFRVIII, CD123, or VEGF-R2. In certain embodiments, the chimeric antigen receptor is an engineered T-cell receptor. In certain embodiments, the chimeric antigen receptor comprises an antigen binding domain comprising a sequence that is at least 90% identical to any one of SEQ ID NOs: 37-56. In certain embodiments, the chimeric antigen receptor comprises an antigen binding domain comprising a sequence that is at least 90% identical to SEQ ID NO: 35 or 36. In certain embodiments, the effective amount of engineered T-cells is at least 10² cells/kg, at least 10⁴ cells/kg, or at least 10⁵ cells/kg. In certain embodiments, the engineered T-cells further express a cytokine, for example a fusion protein comprising IL-15 and IL-15Rα.

Also provided is a use of a fusion protein of the present invention, or a polynucleotide encoding the same, in the manufacture of a medicament for use in the treatment of cancer.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic of PD-1/PD-L1 in immunosuppression.

FIG. 2 is a schematic of TGF-β in immunosuppression.

FIG. 3 shows TGF-β associated gene cluster correlated with metastatic disease and poor prognosis in subset of ovarian cancer patients (enriched in Stage III/IV).

FIG. 4A, FIG. 4B, and FIG. 4C show a schematic design of antibody-TGFRII fusion protein design. In other exemplary embodiments, ADA2 can be fused to the antibody.

FIG. 5 is a graph showing blockade of PD-1/PD-L1 interaction by anti-PD-1 (VH6-VL5) IgG1-TGFβRII and anti-PD-1 (VH6-VL5) IgG4-TGFβRII.

FIG. 6 is a graph showing neutralization of TGF-β1 isoform signaling by anti-PD-1 (VH6-VL5) IgG1-TGFβRII and anti-PD-1 (VH6-VL5) IgG4-TGFβRII.

FIG. 7 is a graph showing neutralization of TGF-β2 isoform by anti-PD-1 (VH6-VL5) IgG1-TGFβRII and anti-PD-1 (VH6-VL5) IgG4-TGFβRII.

FIG. 8 is a graph showing neutralization of TGF-β3 isoform signaling by anti-PD-1 (VH6-VL5) IgG1-TGFβRII and anti-PD-1 (VH6-VL5) IgG4-TGFβRII.

FIG. 9A, FIG. 9B, and FIG. 9C are graphs showing enhanced proliferation and IFN-γ production by stimulated PBMCs in the presence of anti-PD-1-TGFRII fusion protein in a dose dependent manner compared to anti-PD-1 or control antibodies.

FIG. 9D and FIG. 9E are graphs showing PD-1 receptor occupancy on CD8+ T cells and IFN-γ production respectively when anti-PD-1-TGFRII fusion protein is added to co-culture of PBMC and colorectal cancer (colorectal adenocarcinoma) cell line.

FIG. 9F and FIG. 9G are graphs showing PD-1 receptor occupancy on CD8+ T cells and IFN-γ production respectively when anti-PD-1-TGFRII fusion protein is added to co-culture of PBMC and head and neck cancer (pharyngeal carcinoma) cell line.

FIG. 9H and FIG. 9I are graphs depicting TGF-β1 and TGF-β2 concentration respectively in supernatant of PBMC and colorectal cancer (colorectal adenocarcinoma) cells co-culture in presence of anti-PD-1-TGFRII fusion protein.

FIG. 9J and FIG. 9K are graphs depicting TGF-β1 and TGF-β2 concentration respectively in supernatant of PBCM and head and neck cancer (pharyngeal carcinoma) cells co-culture in presence of anti-PD-1-TGFRII fusion protein.

FIG. 9L and FIG. 9M are graphs depicting IFN-γ production respectively in 3D spheroid culture of colorectal cancer (colorectal adenocarcinoma) cells or head & neck cancer (pharyngeal carcinoma) cells and PBMC in presence of anti-PD-1-TGFRII fusion protein as compared to anti-PD-1 alone.

FIGS. 10A-10D show effect of anti-PD-1-TGFRII fusion protein treatment on T cell proliferation and activation in the presence of recombinant TGF-β1.

FIGS. 11A-11F show expression of various cytokines by PBMC in the presence recombinant TGF-β1 and in the presence of anti-PD-1, anti-PD-1-TGFRII fusion protein or control antibodies.

FIG. 12A shows the effect of anti-PD-1-TGFRII fusion protein on tumor growth as compared to anti-PD-1 alone in a humanized mouse model of colorectal cancer.

FIG. 12B shows that treatment with anti-PD-1-TGFRII fusion protein significantly increases CD8+ T cell to Treg ratio in tumor in a humanized mouse model of colorectal cancer.

FIG. 12C shows the effect of anti-PD-1-TGFRII fusion protein treatment on perforin expression levels as compared to anti-PD-1 treatment in a humanized mouse model of colorectal cancer.

FIG. 12D and FIG. 12E shows the effect of anti-PD-1-TGFRII fusion protein treatment on TGF-β1 and TGF-β2 concentrations as compared to anti-PD-1 treatment respectively in a humanized mouse model of colorectal cancer.

FIG. 13A shows treatment with anti-PD-1-TGFRII fusion protein significantly improved IFNγ production compared to anti-PD-1 treatment in an in vitro model of head and neck cancer. FIGS. 13B-13G show treatment with anti-PD-1-TGFRII fusion protein significantly increases T-cell function as evidenced by expression analysis of various pathway genes.

FIG. 14A shows the effect of anti-PD-1-TGFRII fusion protein on tumor growth as compared to anti-PD-1 alone in a humanized mouse model of head and neck cancer.

FIG. 14B shows survival of tumor bearing mice in humanized mouse model of head and neck cancer when treated with anti-PD-1-TGFRII fusion protein vs. anti-PD-1 alone or isotype control.

FIG. 14C shows ratio of CD8+ T cells to regulatory T cells in tumors of mice treated with anti-PD-1-TGFRII fusion protein in a humanized mouse model of head and neck cancer.

FIG. 14D and FIG. 14E shows the effect of anti-PD-1-TGFRII fusion protein on TGF-β1 and TGF-β2 concentrations as compared to anti-PD-1 alone in a humanized mouse model of head and neck cancer.

FIG. 14F shows the effect of anti-PD-1-TGFRII fusion protein on IFN-γ production in a humanized mouse model of head and neck cancer.

FIGS. 15A-15B shows the effect of anti-PD-1 (VH7/VL6)-TGFRII fusion protein on IFN-γ production and TGF-β1 concentration in primary colorectal cancer patient samples.

FIG. 15C shows the gene expression analysis of primary colorectal cancer patient samples co-cultured with anti-PD-1 (VH7/VL6)-TGFRII fusion protein.

FIG. 16 shows a graph depicting the results of a cytotoxicity assay of anti-PD-1 (VH7/VL6)-TGFRII fusion protein compared to anti-PD-L1-TGFRII fusion protein.

FIG. 17 is a graph depicting the results of a cytotoxicity assay of anti-PD-1 (VH7/VL6)-TGFRII fusion protein in combination with a chimeric receptor antigen (CAR) T cells vs. anti-PD-1 in combination with a CAR T cells vs. CAR T cells alone.

FIG. 18A and FIG. 18B are graphs depicting the results of a cytotoxicity assay of anti-PD-1 (VH6/VL5)-TGFRII fusion protein in combination with CD33 CAR-T and a cytotoxicity assay of anti-PD-1 (VH7/VL6)-TGFRII fusion protein in combination with CD33 CAR-T respectively.

FIG. 19A and FIG. 19B are graphs depicting tumor cell lysis using anti-PD-1 (VH6/VL5)-TGFRII fusion protein and anti-PD-1 (VH7/VL6)-TGFRII fusion protein respectively when co-cultured with NK cells.

FIG. 20 is a graph depicting Biacore analysis of simultaneous binding of TGF-b1 and PD-1 by anti-PD-1 (VH6/VL5)-TGFRII fusion proteins using different linkers.

FIG. 21 is a graph showing blockade of PD-1/PD-L1 interaction by anti-PD-1 IgG4-ADA2.

FIG. 22 is a graph showing ADA2 enzymatic activity measured for anti-PD-1 hlgG1-ADA2 and anti-PD-1 hlgG4-ADA2.

FIGS. 23A-23C are graphs showing the effect of various variants of anti-PD-1 and anti-PD-1-ADA2 fusion proteins on PD-L1/PD-1 interaction.

FIGS. 24A-24F are graphs showing the enzymatic activity of various variants of anti-PD-1-ADA2 fusion proteins as measured by ADA enzymatic activity.

FIG. 25 is a graph showing the enzymatic activity of anti-PD-1-mutADA2 vs. anti-PD-1-wtADA2 as measured by ADA enzymatic activity.

FIG. 26A-26D are graphs depicting the effect of variants of anti-PD-1-wtADA2 on T cell proliferation.

FIG. 26E is a graph depicting IFNγ production by anti-PD-1-wtADA2 as compared to anti-PD-1 or isotype control.

FIGS. 27A-27B are graphs depicting the effectiveness of wtADA2 and mutADA2 to reverse adenosine-mediated suppression of T cell proliferation.

FIG. 28 is a graph depicting the effect of variants of anti-PD-1-ADA2 fusion proteins on the blockade of PD-1-PDL1 interaction.

FIG. 29 a graph showing the enzymatic activity of anti-PD-1-ADA2-scFv-Fc as measured by ADA enzymatic activity.

FIG. 30 is a bar graph showing the enzymatic activity of variants of anti-PD-1-ADA2 as measured by ADA enzymatic activity.

FIGS. 31A-31B are graphs depicting the effect of anti-PD-1-wtADA2 on IFN-γ production and proliferation of tumor-infiltrating lymphocytes (TILs) in primary CRC patient tumor.

FIG. 32 is a graph depicting the effect of anti-PD-1 vs. anti-PD-1-wtADA2 on tumor volume of a humanized mouse model of lung cancer.

FIGS. 33A-33C show a schematic design of anti-PD-1-adenosine deaminase 2 (ADA2) design: anti-PD-1-ADA2.

FIG. 34 is a graph depicting the effect of an anti-PD-1-mutADA2 on the blockade of PD-1-PD-L1 interaction.

FIGS. 35A-35B are graphs depicting anti-PD-1-wtADA2 catalyzing adenosine into inosine in both a dose-dependent (FIG. 35A) and a substrate concentration-dependent (FIG. 35B) manner.

FIG. 36A-36C are graphs depicting anti-PD-1-wtADA2 and anti-PD-1-mutADA2 reversing the adenosine-mediated suppression of T cell proliferation in a dose-dependent manner.

FIGS. 37A-37C are graphs depicting anti-PD-1-wtADA2 reversing, in a dose-dependent manner, the inhibition of T cell function (IFN-γ production) mediated by adenosine in three different tumor cell lines (A549, SKOV3, and Panc.08).

FIG. 38 is a graph depicting anti-PD-1-wtADA2 inhibiting growth of an A549 tumor in a NOD scid gamma (“NSG”) mouse.

FIG. 39 is a graph depicting simultaneous binding of TGF-β1 and MUC 16 to anti-MUC16 V_{L}10/V_{H}10-TGFβRII fusion protein.

FIG. 40 is a graph showing binding of a TGF-β1 isoform to an anti-MUC16 V_{L}10/V_{H}10-TGFβRII fusion protein.

### DETAILED DESCRIPTION OF THE DISCLOSURE

The following description and examples illustrate certain embodiments of the present invention.

It is to be understood that the present disclosure is not limited to the particular embodiments described herein and as such can vary. Those of skill in the art will recognize that there are variations and modifications of the present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### Definitions

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include,” “includes,” and “included,” is not limiting.

Reference in the specification to “some embodiments,” “an embodiment,” “one embodiment,” “certain embodiments,” “other embodiments,” or “another embodiment” means that a particular feature, structure, or characteristic described in connection with the embodiment is included in at least one embodiment of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

“Polynucleotide” or “oligonucleotide” as used herein refers to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs.

“Transfection,” “transformation,” or “transduction” as used herein refer to the introduction of one or more exogenous polynucleotides into a host cell by using physical or chemical methods. The polynucleotide sequences and vectors disclosed or contemplated herein can be introduced into a cell by, for example, transfection, transformation, or transduction. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346:776-777 (1990)); and strontium phosphate DNA co-precipitation (Brash et al., Mol. Cell Biol., 7:2031-2034 (1987)). Phage or viral vectors can be introduced into host cells, after growth of infectious particles in suitable packaging cells, many of which are commercially available.

“Polypeptide,” “peptide,” and their grammatical equivalents as used herein refer to a polymer of amino acid residues. The polypeptide can optionally include glycosylation or other modifications typical for a given protein in a given cellular environment. Polypeptides and proteins disclosed herein (including functional portions and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides or proteins described herein in an engineered cell can be associated with post-translational modifications of one or more amino acids of the polypeptide or protein. Non-limiting examples of post-translational modifications include phosphorylation, acylation including acetylation and formylation, glycosylation (including N-linked and O-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

The term “fragment” when used in referece to a polypeptide or a nucleic acid refers to a truncated polypeptide or nucleic acid that retains the primary function of the referenced polypeptide or nucleic acid.

The term “variant” when used in referece to a polypeptide or a nucleic acid refers to a polypeptide or nucleic acid that differs from the parent polypeptide or nucleic acid but has at least 50% sequence identity with the referenced polypeptide or nucleic acid and retains the primary function of the parent polypeptide or nucleic acid.

The terms “identical” and its grammatical equivalents as used herein or “sequence identity” in the context of two nucleic acid sequences or amino acid sequences of polypeptides refer to the residues in the two sequences which are the same when aligned for maximum correspondence over a specified comparison window. A “comparison window,” as used herein, refers to a segment of at least about 20 contiguous positions, usually about 50 to about 200, more usually about 100 to about 150 in which a sequence can be compared to a reference sequence of the same number of contiguous positions after the two sequences are aligned optimally. Methods of alignment of sequences for comparison are well-known in the art. Optimal alignment of sequences for comparison can be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein are at least 80%, 85%, 90%, 95%, 98% 99% or 100% identical to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 95%, 98%, 99% or 100% identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, the percentage of residues in the smaller molecule finds a match residue in the larger molecule in accordance with the order by which the two molecules are optimally aligned.

The term “substantially identical” and its grammatical equivalents as applied to nucleic acid or amino acid sequences mean that a nucleic acid or amino acid sequence comprises a sequence that has at least 90% sequence identity or more, at least 95%, at least 98% and at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In some embodiments, the substantial identity exists over a region of the sequences that is at least about 50 residues in length, over a region of at least about 100 residues, and in some embodiments, the sequences are substantially identical over at least about 150 residues. In some embodiments, the sequences are substantially identical over the entire length of the coding regions.

“Homology” is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available. Nucleic acids and/or nucleic acid sequences are “homologous” when they are derived, naturally or artificially, from a common ancestral nucleic acid or nucleic acid sequence. Proteins and/or protein sequences are “homologous” when their encoding DNAs are derived, naturally or artificially, from a common ancestral nucleic acid or nucleic acid sequence. The homologous molecules can be termed “homologs.” For example, any naturally occurring proteins can be modified by any available mutagenesis method. When expressed, this mutagenized nucleic acid encodes a polypeptide that is homologous to the protein encoded by the original nucleic acid.

The term “isolated” and its grammatical equivalents as used herein refer to the removal of a nucleic acid from its natural environment. The term “purified” and its grammatical equivalents as used herein refer to a molecule or composition, whether removed from nature (including genomic DNA and mRNA) or synthesized (including cDNA) and/or amplified under laboratory conditions, that has been increased in purity, wherein “purity” is a relative term, not “absolute purity.” It is to be understood, however, that nucleic acids and proteins can be formulated with diluents or adjuvants and still for practical purposes be isolated. For example, nucleic acids typically are mixed with an acceptable carrier or diluent when used for introduction into cells. The term “substantially purified” and its grammatical equivalents as used herein refer to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

An “expression vector” or “vector” is any genetic element, e.g., a plasmid, chromosome, virus, transposon, behaving either as an autonomous unit of polynucleotide replication within a cell. (i.e. capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, transposons, bacteriophages and cosmids. Vectors can contain polynucleotide sequences which are necessary to effect ligation or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors can be capable of directly expressing nucleic acid sequence products encoded therein without ligation or integration of the vector into host cell DNA sequences. In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11:1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.) and pBK-CMV from Stratagene (La Jolla, Calif.) represent non-limiting examples of an episomal vector that uses T-antigen and the SV40 origin of replication in lieu of EBNA1 and oriP. Vector also can comprise a selectable marker gene.

The term “selectable marker gene” as used herein refers to a nucleic acid sequence that allows cells expressing the nucleic acid sequence to be specifically selected for or against, in the presence of a corresponding selective agent. Suitable selectable marker genes are known in the art and described in, e.g., International Patent Application Publications WO 1992/08796 and WO 1994/28143; Wigler et al., Proc. Natl. Acad. Sci. USA, 77:3567 (1980); O'Hare et al., Proc. Natl. Acad. Sci. USA, 78:1527 (1981); Mulligan & Berg, Proc. Natl. Acad. Sci. USA, 78:2072 (1981); Colberre-Garapin et al., J. Mol. Biol., 150:1 (1981); Santerre et al., Gene, 30:147 (1984); Kent et al., Science, 237:901-903 (1987); Wigler et al., Cell, 11:223 (1977); Szybalska & Szybalski, Proc. Natl. Acad. Sci. USA, 48:2026 (1962); Lowy et al., Cell, 22:817 (1980); and U.S. Pat. Nos. 5,122,464 and 5,770,359.

The term “coding sequence” as used herein refers to a segment of a polynucleotide that encodes for protein or polypeptide. The region or sequence is bounded nearer the 5′ end by a start codon and nearer the 3′ end with a stop codon. Coding sequences can also be referred to as open reading frames.

The term “operably linked” as used herein refers to refers to the physical and/or functional linkage of a DNA segment to another DNA segment in such a way as to allow the segments to function in their intended manners. A DNA sequence encoding a gene product is operably linked to a regulatory sequence when it is linked to the regulatory sequence, such as, for example, promoters, enhancers and/or silencers, in a manner which allows modulation of transcription of the DNA sequence, directly or indirectly. For example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. An enhancer or silencer is operably linked to a DNA sequence coding for a gene product when it is ligated to the DNA sequence in such a manner as to increase or decrease, respectively, the transcription of the DNA sequence. Enhancers and silencers can be located upstream, downstream or embedded within the coding regions of the DNA sequence. A DNA for a signal sequence is operably linked to DNA coding for a polypeptide if the signal sequence is expressed as a pre-protein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

The terms “induce” and “induction,” and their grammatical equivalents as used herein, refer to an increase in nucleic acid sequence transcription, promoter activity and/or expression brought about by a transcriptional regulator, relative to some basal level of transcription.

The term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus (such enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

The term “promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. The term “promoter activity” and its grammatical equivalents as used herein refer to the extent of expression of nucleotide sequence that is operably linked to the promoter whose activity is being measured. Promoter activity can be measured directly by determining the amount of RNA transcript produced, for example by Northern blot analysis or indirectly by determining the amount of product coded for by the linked nucleic acid sequence, such as a reporter nucleic acid sequence linked to the promoter.

“Inducible promoter” as used herein refers to a promoter which is induced into activity by the presence or absence of transcriptional regulators, e.g., biotic or abiotic factors. Inducible promoters are useful because the expression of genes operably linked to them can be turned on or off at certain stages of development of an organism or in a particular tissue. Non-limiting examples of inducible promoters include alcohol-regulated promoters, tetracycline-regulated promoters, steroid-regulated promoters, metal-regulated promoters, pathogenesis-regulated promoters, temperature-regulated promoters and light-regulated promoters. The inducible promoter can be part of a gene switch or genetic switch.

“T cell” or “T lymphocyte” as used herein is a type of lymphocyte that plays a central role in cell-mediated immunity. They can be distinguished from other lymphocytes, such as B cells and natural killer cells (NK cells), by the presence of a T-cell receptor (TCR) on the cell surface.

The term “antibody,” also known as immunoglobulin (lg), as used herein can refer to a monoclonal or polyclonal antibody. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. The antibodies can be from any animal origin. An antibody can be IgG (including IgG1, IgG2, IgG3, and IgG4), IgA (including IgA1 and IgA2), IgD, IgE, or IgM, and IgY. In some embodiments, the antibody can be whole antibodies, including single-chain whole antibodies. In some embodiments, the antibody can be a fragment of an antibody, which can include, but are not limited to, a Fab, a Fab′, a F(ab′)₂, a Fd (consisting of V_{H} and CH1), a Fv fragment (consisting of V_{H} and V_{L}), a single-chain variable fragment (scFv), a single-chain antibody, a disulfide-linked variable fragment (dsFv), and fragments comprising either a V_{L}, or V_{H} domain. A whole antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The V_{H} and V_{L}, regions have a similar general structure, with each region comprising four framework regions, whose sequences are relatively conserved. The framework regions are connected by three complementarity determining regions (CDRs). The three CDRs, known as CDRI, CDR2, and CDR3, form the “hypervariable region” of an antibody, which is responsible for antigen binding. These particular regions have been described by Kabat et al., J. Biol. Chem. 252, 6609-6616 (1977) and Kabat et al., Sequences of protein of immunological interest. (1991), by Chothia et al., J. Mol. Biol. 196:901-917 (1987), and by MacCallum et al., J. Mol. Biol. 262:732-745 (1996), all of which are herein incorporated by reference in their entireties, where the definitions include overlapping or subsets of amino acid residues when compared against each other. Preferably, the term “CDR” is a CDR as defined by Kabat, based on sequence comparisons. CDRH1, CDRH2 and CDRH3 denote the heavy chain CDRs, and CDRL1, CDRL2 and CDRL3 denote the light chain CDRs.

The terms “fragment of an antibody,” “antibody fragment,” “fragment of an antibody,” “antigen-binding portion” or its grammatical equivalents are used interchangeably herein to mean one or more fragments or portions of an antibody that retain the ability to specifically bind to an antigen (see, generally, Holliger et al., Nat. Biotech., 23 (9): 1126-1129 (2005)). The antibody fragment desirably comprises, for example, one or more CDRs, the variable region (or portions thereof), the constant region (or portions thereof), or combinations thereof. Non-limiting examples of antibody fragments include (1) a Fab fragment, which is a monovalent fragment consisting of the V_{L}, V_{H}, CL, and CHI domains; (2) a F(ab′)2 fragment, which is a bivalent fragment comprising two Fab fragments linked by a disulfide bridge at the stalk region; (3) a Fv fragment consisting of the V_{L}, and V_{H} domains of a single arm of an antibody; (4) a single chain Fv (scFv), which is a monovalent molecule consisting of the two domains of the Fv fragment (i.e., V_{L} and V_{H}) joined by a linker which enables the two domains to be synthesized as a single polypeptide chain (see, e.g., Bird et al., Science, 242:423-426 (1988); Huston et al., Proc. Natl. Acad. Sci. USA, 85:5879-5883 (1988); and Osbourn et al., Nat. Biotechnol., 16:778 (1998)) and (5) a diabody, which is a dimer of polypeptide chains, wherein each polypeptide chain comprises a V_{H} connected to a V_{L} by a peptide linker that is too short to allow pairing between the V_{H} and V_{L}, on the same polypeptide chain, thereby driving the pairing between the complementary domains on different V_{H}-V_{L} polypeptide chains to generate a dimeric molecule having two functional antigen binding sites. Antibody fragments are known in the art and are described in more detail in, e.g., U.S. Pat. No. 8,603,950.

“Antigen recognition moiety,” “antigen recognition domain,” “antigen binding domain,” or “antigen binding region” refers to a molecule or portion of a molecule that specifically binds to an antigen. In some embodiments, the antigen recognition moiety is an antibody, antibody like molecule or fragment thereof.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups above, for example, lysine for arginine and vice versa such that a positive charge can be maintained; glutamic acid for aspartic acid and vice versa such that a negative charge can be maintained; serine for threonine such that a free-OH can be maintained; and glutamine for asparagine such that a free —NH₂ can be maintained. Alternatively or additionally, the functional variants can comprise the amino acid sequence of the reference protein with at least one non-conservative amino acid substitution.

The term “non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution can enhance the biological activity of the functional variant, such that the biological activity of the functional variant is increased as compared to the homologous parent protein.

The term “proliferative disease” as referred to herein refers to a unifying concept in which excessive proliferation of cells and/or turnover of cellular matrix contributes significantly to the pathogenesis of the disease, including cancer. In some embodiments, the proliferative disease is cancer.

“Patient” or “subject” as used herein refers to a mammalian subject diagnosed with or suspected of having or developing a proliferative disorder such as cancer. In some embodiments, the term “patient” refers to a mammalian subject with a higher than average likelihood of developing a proliferative disorder such as cancer. Exemplary patients can be humans, apes, dogs, pigs, cattle, cats, horses, goats, sheep, rodents and other mammalians that can benefit from the therapies disclosed herein. Exemplary human patients can be male and/or female. “Patient in need thereof” or “subject in need thereof” is referred to herein as a patient diagnosed with or suspected of having a disease or disorder, for instance, but not restricted to cancer.

“Administering” is referred to herein as providing one or more compositions described herein to a patient or a subject. By way of example and not limitation, composition administration, e.g., injection, can be performed by intravenous (i.v.) injection, subcutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise a target cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

As used herein, the term “treatment,” “treating,” or its grammatical equivalents refers to obtaining a desired pharmacologic and/or physiologic effect. In embodiments, the effect is therapeutic, i.e., the effect partially or completely cures a disease and/or adverse symptom attributable to the disease. In some embodiments, the term “treating” can include “preventing” a disease or a condition.

As used herein, a “treatment interval” refers to a treatment cycle, for example, a course of administration of a therapeutic agent that can be repeated, e.g., on a regular schedule. In embodiments, a dosage regimen can have one or more periods of no administration of the therapeutic agent in between treatment intervals. For example, a treatment interval can include one dose of a fusion protein administered in combination with (prior, concurrently or after) administration of a second therapeutic agent, e.g. CAR-T cells.

The terms “administered in combination” or “co-administration” or “co-administering” or “co-providing” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the first treatment and second treatment can be administered simultaneously (e.g., at the same time), in the same or in separate compositions, or sequentially. Sequential administration refers to administration of one treatment before (e.g., immediately before, less than 5, 10, 15, 30, 45, 60 minutes; 1, 2, 3, 4, 6, 8, 10, 12, 16, 20, 24, 48, 72, 96 or more hours; 4, 5, 6, 7, 8, 9 or more days; 1, 2, 3, 4, 5, 6, 7, 8 or more weeks before) administration of an additional, e.g., secondary, treatment. The order of administration of the first and secondary treatment can also be reversed.

The terms “therapeutically effective amount,” therapeutic amount,” “immunologically effective amount,” “anti-tumor effective amount,” “tumor-inhibiting effective amount,” and their grammatical equivalents, refer to an amount effective, at dosages and for periods of time necessary, to achieve a desired therapeutic result. The therapeutically effective amount can vary according to factors such as the disease state, age, sex, and weight of the individual, and the ability of a composition described herein to elicit a desired response in one or more subjects. The precise amount of the compositions of the present disclosure to be administered can be determined by a physician with consideration of individual differences in age, weight, tumor size, extent of infection or metastasis, and condition of the patient (subject).

Alternatively, the pharmacologic and/or physiologic effect of administration of one or more compositions described herein to a patient or a subject of can be “prophylactic,” i.e., the effect completely or partially prevents a disease or symptom thereof. A “prophylactically effective amount” refers to an amount effective, at dosages and for periods of time necessary, to achieve a desired prophylactic result (e.g., prevention of disease onset).

As used herein, reference to a particular polypeptide (e.g., antibody, ligand, or receptor) shall include any and all forms of that polypeptide, including fragments and variants that retain at least part of the activity of the polypeptide. Unless indicated differently, reference to a particular polypeptide includes all mammalian species thereof (e.g., human, canine, feline, equine, and bovine). For example, the term “PD-1” necessarily includes any active form of PD-1, including fragments and variants that retain at least partial PD-1 activity.

### Fusion Proteins

In some embodiments, the fusion protein (or fragment or variant thereof) provided herein comprise: (a) a first component that is an antibody, or a fragment or variant thereof; and (b) a second component this is a cytokine trap or an adenosine deaminase (ADA) or a fragment or variant thereof. In certain embodiments, the antibody, or fragment or variant thereof, of the first component binds to programmed cell death protein-1 (PD-1). Such an antibody will be referred to herein as an “anti-PD-1 antibody.” In certain other embodiments, the antibody, or fragment or variant thereof, of the first component binds to a tumor antigen expressed on the surface of a tumor cell.

In some embodiments of the fusion protein comprising an anti-PD-1 antibody, the fusion protein comprises prembrolizumab (Ketruda®) fused to a cytokine trap (e.g., TGF-β trap). In some embodiments, the fusion protein comprises nivolumab (Opdivo®) fused to a cytokine trap (e.g., TGF-β trap). In some embodiments, the fusion protein comprises zimberelimab (WBP3055) fused to a cytokine trap (e.g., TGF-β trap). In some embodiments, the fusion protein comprises cetrelimab (JNJ-63723283) can be fused to a cytokine trap (e.g., TGF-β trap).

In certain embodiments wherein the antibody, or fragment or variant thereof, binds to a tumor antigen expressed on the surface of a tumor cell, the tumor antigen may be a MUCI antigen. In certain other such embodiments, the tumor antigen is a MUC16 antigen.

In some embodiments, the fusion protein (or fragment or variant thereof) described herein comprises a cytokine trap as described above and an antibody, a fragment or a variant of the antibody that targets immune checkpoint genes. In some embodiments, an antibody or a fragment of the antibody or a variant of the antibody that target immune checkpoints, such as cytotoxic T lymphocyte associated protein-4 (CTLA-4) and programmed cell death-ligand-1 (PDL1), can be fused to a TGF-β trap molecule via a linker. In some embodiments, the PD-L1 inhibitor is atezolizumab.

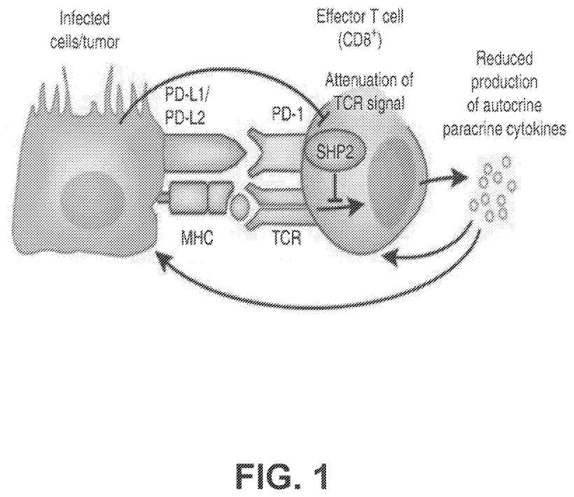

U.S. patent application publication number U.S. 2015/0225483A1, incorporated herein by reference, describes a bi-functional fusion protein that combines a PD-L1 antibody with the soluble extracellular domain of transforming growth factor beta receptor type II (TGFβRII) as a neutralizing “Trap,” into a single molecule. Specifically, the protein is a heterotetramer, consisting of the two immunoglobulin light chains of anti-PD-L1, and two heavy chains comprising the heavy chain of anti-PD-L1 genetically fused via a flexible glycine-serine linker to the extracellular domain of the human TGFβRII (sce FIG. 1). This anti-PD-L1/TGF′P Trap molecule is designed to target two major mechanisms of immunosuppression in the tumor microenvironment. US patent application publication number US2015/0225483A1 describes administration of the Trap molecule at doses based on the patient's weight.

In some embodiments, the fusion protein (or fragment or variant thereof) provided herein comprises an anti-PD-1 from Table 1 fused to an adenosine deaminase (e.g., ADA2) described herein or a functional variant or derivative thereof. In some embodiments, the fusion protein comprises prembrolizumab (Keytruda®) fused to an adenosine deaminase (e.g., ADA2). In some embodiments, the fusion protein comprises nivolumab (Opdivo®) fused to an adenosine deaminase (e.g., ADA2). In some embodiments, the fusion protein comprises zimberelimab (WBP3055) fused to an adenosine deaminase (e.g., ADA2). In some embodiments, the fusion protein comprises cetrelimab (JNJ-63723283) fused to an adenosine deaminasc (e.g., ADA2).

### Programmed Cell Death Protein and Other Check-Point Inhibitors

Programmed cell death protein 1, also known as PD-1, is a family of inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells. For purposes of the present invention, it will be explicitly understood that the term “PD-1” encompass all known and previously identified terms and titles of PD-1, including, for example, PD-1, PD-1, programmed cell death protein 1, programmed cell death 1, programmed cell death 1 receptor, programmed cell death 1 protein, CD279, cluster of differentiation 279, hPD-1, PDCD1, CD279 antigen, systemic lupus erythematosus susceptibility 2, HPD-L, HSLE1, and/or SLEB2.

PD-1 was previously identified using a subtraction cloning based approach to select for genes upregulated during TCR-induced activated T cell death. Like CTLA-4, PD-1 is rapidly induced on the surface of T-cells in response to anti-CD3 (Agata et al., 25 Int. Immunol. 8:765 (1996)). In contrast to CTLA-4, however, PD-1 is also induced on the surface of B-cells (in response to anti-IgM). PD-1 is also expressed on a subset of thymocytes and myeloid cells (Agata et al., supra; Nishimura et al., Int. Immunol. 8:773 (1996)).

The nucleic acid and amino acid sequences of a representative human PD-1 biomarker is available to the public at the GenBank database under NM_005018.2 and NP_005009.2 and is shown in Table 26 (see also Ishida et al. 20 EMBO J 11:3887 (1992); Shinohara et al. Genomics 23:704 (1994); U.S. Pat. No. 5,698,520). PD-1 has an extracellular region containing immunoglobulin superfamily domain, a transmembrane domain, and an intracellular region including an immunoreceptor tyrosine-based inhibitory motif (ITIM) (Ishida et al., EMBO J. 11:3887 (1992); Shinohara et al., Genomics 23:704 (1994); and U.S. Pat. No. 5,698,520) and an immunoreceptor tyrosine-based switch motif (ITSM). These features also define a larger family of polypeptides, called the immunoinhibitory receptors, which also includes gp49B, PIR-B, and the killer inhibitory receptors (KIRs) (Vivier and Daeron, Immunol. Today 18:286 (1997)). It is often assumed that the tyrosyl phosphorylated ITIM and ITSM motif of these receptors interacts with SH2-domain containing phosphatases, which leads to inhibitory signals. A subset of these immunoinhibitory receptors bind to MHC polypeptides, for example the KIRs, and CTLA4 binds to B7-1 and B7-2. It has been proposed that there is a phylogenetic relationship between the MHC and B7 genes (Henry et al., Immunol. Today 20 (6): 285-8 (1999)). Nucleic acid and polypeptide sequences of PD-1 orthologs in organisms other than humans are well known and include, for example, mouse PD-1 (NM_008798.2 and NP_032824.1), rat PD-1 (NM_001106927.1 and NP_001100397.1), dog PD-1 (XM_543338.3 and XP_543338.3), cow PD-1 (NM_001083506.1 and NP_001076975.1), and chicken PD-1 (XM_422723.3 and XP_422723.2).

As used herein, the term “anti-PD-1” is used synonymously with “PD-1 antibody”, “anti-PD-1 antibody” and “PD-1 inhibitor,” and each of these terms refers to a polypeptide capable of inhibiting PD-1 biological activity and/or downstream event(s) mediated by PD-1. For clarity, the term “anti-PD-1” encompasses anti-PD-1 proteins and fragments and variants thereof that are capable of inhibiting PD-1 biological activity and/or downstream event(s) mediated by PD-1. Anti-PD-1 proteins encompass polypeptides that block, antagonize, suppress or reduce (to any degree including significantly) PD-1 biological activity, including downstream events mediated by PD-1, such as PD-1 binding and downstream signaling, inhibition of T cell proliferation, inhibition of T cell activation, inhibition of IFN secretion, inhibition of IL-2 secretion, inhibition of TNF secretion, induction of IL-10, and inhibition of anti-tumor immune responses. For purposes of the present invention, it will be explicitly understood that the terms “anti-PD-1, “PD-1 antibody,” “anti-PD-1 antibody,” and “PD-1 inhibitor” encompass all the previously identified terms, titles, and functional states and characteristics whereby PD-1 itself or a PD-1 biological activity, is substantially nullified, decreased, or neutralized in any meaningful degree. In some embodiments, an anti-PD-1 antibody binds PD-1 and upregulates anti-tumor immune response.

As used herein, the terms “PD-1 ligand” and “PD-L” refer to binding partners of the PD-1 receptor and includes all known PD-1 ligands polypeptides, including PD-L1 (Freeman et al., J. Exp. Med. 192:1027 (2000)) and PD-L2 (Latchman et al., Nat. Immunol. 2: flinker (2001)). PD-1 ligand proteins comprise a signal sequence, and an IgV domain, an IgC domain, a transmembrane domain, and a short cytoplasmic tail. Both PD-L1 (see, e.g., Freeman et al., J. Exp. Med. 192:1027 (2000) for sequence data) and PD-L2 (see, e.g., Latchman et al., Nat. Immunol. 2:261 (2000) for sequence data) are members of the B7 family of polypeptides. Both PD-L1 and PD-L2 are expressed in placenta, spleen, lymph nodes, thymus, and heart. Only PD-L2 is expressed in pancreas, lung and liver, while only PD-L1 is expressed in fetal liver. Both PD-1 ligands are upregulated on activated monocytes and dendritic cells, although PD-L1 expression is broader. For example, PD-L1 is known to be constitutively expressed and upregulated to higher levels on murine hematopoietic cells (e.g., T cells, B cells, macrophages, dendritic cells (DCs), and bone marrow-derived mast cells) and non-hematopoietic cells (e.g., endothelial, epithelial, and muscle cells), whereas PD-L2 is inducibly expressed on DCs, macrophages, and bone marrow-derived mast cells (see, e.g., Butte et al., Immunity 27:111 (2007)).

PD-1 ligands comprise a family of polypeptides having certain conserved structural and functional features. The term “family” when used to refer to proteins or nucleic acid molecules, is intended to mean two or more proteins or nucleic acid molecules having a common structural domain or motif and having sufficient amino acid or nucleotide sequence homology, as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics. PD-1 ligands are members of the B7 family of polypeptides. The term “B7 family” or “B7 polypeptides” as used herein includes costimulatory polypeptides that share sequence homology with B7 polypeptides, e.g., with B7-1, B7-2, B7h (Swallow et al., Immunity 11:423 (1999)), and/or PD-1 ligands (e.g., PD-L1 or PD-L2). For example, human B7-1 and B7-2 share approximately 26% amino acid sequence identity when compared using the BLAST program at NCBI with the default parameters (Blosum62 matrix with gap penalties set at existence 11 and extension 1 (see the NCBI website). The term B7 family also includes variants of these polypeptides which are capable of modulating immune cell function. The B7 family of molecules share a number of conserved regions, including signal domains, IgV domains and the IgC domains. IgV domains and the IgC domains are art-recognized Ig superfamily member domains. These domains correspond to structural units that have distinct folding patterns called Ig folds. Ig folds are comprised of a sandwich of two β sheets, each consisting of anti-parallel β strands of 5-10 amino acids with a conserved disulfide bond between the two sheets in most, but not all, IgC domains of Ig, TCR, and MHC molecules share the same types of sequence patterns and are called the C1-set within the Ig superfamily. Other IgC domains fall within other sets. IgV domains also share sequence patterns and are called V set domains. IgV domains are longer than IgC domains and contain an additional pair of β strands.

Many B7 polypeptides are capable of providing costimulatory or inhibitory signals to immune cells to promote or inhibit immune cell responses. For example, B7 family members that bind to costimulatory receptors increase T cell activation and proliferation, while B7 family members that bind to inhibitory receptors reduce costimulation. Moreover, the same B7 family member may increase or decrease T cell costimulation. For example, when bound to a costimulatory receptor, PD-1 ligand can induce costimulation of immune cells or can inhibit immune cell costimulation, e.g., when present in soluble form. When bound to an inhibitory receptor, PD-1 ligand polypeptides can transmit an inhibitory signal to an immune cell. Preferred B7 family members include B7-1, B7-2, B7h, PD-L1 or PD-L2 and soluble fragments or derivatives thereof. In some embodiments, B7 family members bind to one or more receptors on an immune cell, e.g., CTLA4, CD28, ICOS, PD-1 and/or other receptors, and, depending on the receptor, have the ability to transmit an inhibitory signal or a costimulatory signal to an immune cell, preferably a T cell.

The PD-1/PD-L1 signaling axis can promote tumor mediated immune evasion. In some cases, PD-L1 can be over-expressed by tumor cells, accessory cells, such as myeloid-derived suppressor cell (MDSCs), tumor associated macrophages (TAMs), antigen presenting cells (APCs), in the tumor microenvironment. In some cases, PD-1 can be upregulated by “exhausted” T cells and can signal to suppress effector T cells function upon binding to its ligand (PD-L1, PD-L2, and CD80). Blockade of the PD-1/PD-L1 pathway by anti-PD-1 or anti-PD-L1 can restore the function of exhausted T cells and promote tumor cell killing (FIG. 1).

In some embodiments of the present invention, the PD-1 inhibitor is selected or derived from a full-length, fragment, or variant of an antibody from Table 1.

### Table 1: PD-1 Antibodies

| Name | Also Known as | Company | Reference(s) |
|---|---|---|---|
| cemiplimab | Libtayo, | Regeneron, Sanofi | WO |
| | cemiplimab, | | 2015/112800 |
| | REGN2810 | | |
| pembrolizumab | Keytruda, | Merck (MSD), Schering- | WO |
| | MK-3475, SCH | Plough | 2008/156712, |
| | 900475, | | U.S. Pat. No. 8,354,509, |
| | lambrolizumab | | U.S. Pat. No. 8,952,136, |
| | | | U.S. Pat. No. 8,900,587 |
| nivolumab | Opdivo, | BMS, Medarex, Ono | U.S. Pat. No. 8,728,474, |
| | ONO-4538, | | U.S. Pat. No. 8,779,105, |
| | MDX-1106, | | U.S. Pat. No. 8,008,449, |
| | BMS-936558, | | U.S. Pat. No. 9,067,999, |
| | 5C4 | | U.S. Pat. No. 9,073,994 |
| toripalimab | JS001, JS-001, | Junmeng Biosciences, | Si-Yang Liu et al., |
| | TAB001, | Shanghai Junshi, TopAlliance | J. Hematol. Oncol. |
| | triprizumab | Bio | 10: 136 (2017) |
| sintilimab | Tyvyt, IBI308 | Adimab, Innovent, Lilly | WO |
| | | | 2017/024465, |
| | | | WO |
| | | | 2017/025016, |
| | | | WO |
| | | | 2017/132825, |
| | | | WO |
| | | | 2017/133540 |
| LY3434172 | — | Lilly, Zymeworks | ClinicalTrials.gov |
| | | | Identifier: |
| | | | NCT03936959 |
| JTX-4014 | — | Jounce Therapeutics Inc. | U.S. |
| | | | 2018/0118829; |
| | | | ClinicalTrials.gov |
| | | | Identifier: |
| | | | NCT03790488; |
| | | | Papdopolous et al., |
| | | | Cancer Immunol Immunother |
| | | | 70(3): 763-772 (2021)) |
| 609 A | 609A | 3S Bio; Sunshine Guojian | ClinicalTrials.gov |
| | | Pharma | Identifier: |
| | | | NCT03998345 |
| Sym021 | — | Symphogen A/S | Gjetting et al., |
| | | | mAbs 11(4): |
| | | | 666-680 (2019 |
| LZM009 | — | Livzon Pharmaceutical Group | ClinicalTrials.gov |
| | | | Identifier: |
| | | | NCT03286296 |
| budigalimab | ABBV-181, | Abbvie | Powderly et al., |
| | PR-1648817 | | Annals of Oncology |
| | | | 29(8) (2018); |
| | | | ClinicalTrials.gov |
| | | | Identifier: |
| | | | NCT03000257 |
| IB | IBI-318 | Innovent, Lilly | ClinicalTrials.gov |
| | | | Identifier: |
| | | | NCT03875157 |
| SCT-I10A | — | Sinocelltech Ltd. | ClinicalTrials.gov |
| | | | Identifier: |
| | | | NCT03821363 |
| SG001 | — | CSPC ZhongQi | ClinicalTrials.gov |
| | | Pharmaceutical Technology | Identifier: |
| | | Co., Ltd. | NCT03852823 |
| AMP-224 | GSK-2661380 | Astra Zeneca, Glaxo Smith | Floudas et al., |
| | | Kline | Clin. Colorectal Cancer |
| | | | 18(4) (2019) |
| AMG 404 | AMG404 | Amgen | ClinicalTrials.gov |
| | | | Identifier: |
| | | | NCT03853109 |
| AK112 | — | Akesobio Australia Pty Ltd | ClinicalTrials.gov |
| | | | Identifier: |
| | | | NCT04047290 |
| CS1003 | — | CStone Pharma | Li et al., |
| | | | Acta Pharmacologica Sinica |
| | | | 42: 142-148 (2021) |
| MEDI0680 | AMP-514 | Astra Zeneca, Amplimmune, | WO |
| | | Medimmune | 2012/145493, |
| | | | WO |
| | | | 2014/194293 |
| RO7121661 | — | Roche | ClinicalTrials.gov |
| | | | Identifier: |
| | | | NCT03708328 |
| F520 | — | Shandong New Time | ClinicalTrials.gov |
| | | Pharmaceutical Co. | Identifier: |
| | | | NCT03657381 |
| sasanlimab | PF-06801591, | Pfizer | Cho et al., |
| | RN-888 | | Annals of Oncology |
| | | | 30(5) (2019) |
| BI 754091 | BI754091 | Boehringer Ingelheim | Kang et al., |
| | | | J. Clin. Oncology |
| | | | 38(15) (2020) |
| cetrelimab | JNJ-63723283 | Janssen Biotech | Rutkowski et al., |
| | | | J. Clin. Oncology |
| | | | 37: 8 (2019) |
| HerinCAR- | — | Ningbo Cancer Hospital | ClinicalTrials.gov |
| PD-1 | | | Identifier: |
| | | | NCT02873390 |
| HX008 | — | Taizhou Hanzhong Bio | Zhang et al, |
| | | | mAbs |
| | | | 12(1) (2020); |
| | | | ClinicalTrials.gov |
| | | | Identifier: |
| | | | NCT03704246 |
| zimberelimab | WBP3055, | Arcus, Guangzhou Gloria | US |
| | GLS-010, | Bio, Harbin Gloria Pharma, | 2019/0270815, |
| | AB122 | WuXi Biologies | Si-Yang Liu et al., |
| | | | J. Hematol. Oncol. |
| | | | 10: 136 (2017) |
| retifanlimab | MGA012, | Incyte, MacroGenics | WO |
| | INCMGA00012 | | 2017/19846 |
| balstilimab | AGEN2034, | Agenus, Ludwig Inst., Sloan- | WO |
| | AGEN-2034 | Kettering | 2017/040790 |
| pidilizumab | CT-011, hBat-1, | CureTech, Medivation, Teva | Rosenblatt et al., |
| | MDV9300 | | J Immunother. |
| | | | 34(5): 409-118 |
| | | | (2011) |
| teripalimab | — | Henan Cancer Hospital | ClinicalTrials.gov |
| | | | Identifier: |
| | | | NCT03985670 |
| CBT-501 | GB226, GB 226, | CBT Pharmaceuticals, Genor | ClinicalTrials.gov |
| | Genolimzumab, | | Identifier: |
| | Genormab | | NCT03053466. |
| BAT1306 | — | Bio-Thera Solutions | Wu et al., |
| | | | J. Clin. Oncol. |
| | | | 37(4) (2019) |
| tislelizumab | BGB-A317 | BeiGene, Celgene | WO |
| | | | 2015/35606, |
| | | | US |
| | | | 2015/0079109 |
| AK105 | — | Akeso, HanX Bio | ClinicalTrials.gov |
| | | | Identifier: |
| | | | NCT03866967 |
| spartalizumab | PDR001, | Dana-Farber, Novartis | WO |
| | BAP049 | | 2015/112900; |
| | | | Lin et al., |
| | | | Ann. of Oncology, |
| | | | 29: 8 (2018) |
| prolgolimab | BCD-100 | Biocad | Kaplon et al., |
| | | | mAbs 10(2): |
| | | | 183-203 (2018) |
| serplulimab | HLX10 | Henlix Biotech | ClinicalTrials.gov |
| | | | Identifier: |
| | | | NCT04297995 |
| dostarlimab | ANB011, | AnaptysBio, Tesaro | WO |
| | TSR-042, | | 2014/179664 |
| | ABT1, | | |
| | WBP-285 | | |
| camrelizumab | SHR-1210 | Incyte, Jiangsu Hengrui, | WO |
| | | Shanghai Hengrui | 2015/085847; |
| | | | Si-Yang Liu et al., |
| | | | J. Hematol. Oncol. |
| | | | 10: 136 (2017) |
| IBI319 | IBI-319 | Innovent Biologies (Suzhou) | ClinicalTrials.gov |
| | | Co. Ltd., Lilly | Identifier: |
| | | | NCT04708210 |
| KY1043 | — | Kymab | Van Krinks, |
| | | | Kymab poster |
| | | | no. P625, |
| | | | “KY1043, a |
| | | | novel PD-L1 |
| | | | IL-2 |
| | | | immunocytokine |
| | | | directed towards |
| | | | CD25, delivers |
| | | | potent |
| | | | anti-tumour |
| | | | activity |
| | | | in vitro |
| | | | and in vivo” |
| STI-1110 | — | Sorrento Therapeutics | WO |
| | | | 2014/194302 |
| CA05100948 | Antibody 948 | UCB Biopharma | U.S. Pat. No. 8,993,731 |
| Nb97 | MY2935, | Fudan University, Novamab | Xian et al., |
| | MY2626 | | Biochem. & Biophys. Res. Comm's |
| | | | 519(3), 267-273 (2019) |
| ENUM | — | Enumeral | Scheuplein et al., |
| 388D4 | | | Immunology, |
| | | | Abstract 4871 |
| | | | (2016) |
| hAb-10D3 | hAb-10D3 | BPS Bioscience | U.S. Pat. No. 10,759,859 |
| Unknown | — | Isis Innovation | WO |
| | | | 2010/029434 |
| ANB030 | — | AnaptysBio Inc. | Grebinoski et al., |
| | | | Current Opinion in Immunol. |
| | | | 67: 1-9 (2020) |
| MCLA-134 | — | Merus N.V. | WO |
| | | | 2019/009727 |
| hAb21 | — | Suzhou Stainwei Biotech Inc. | U.S. |
| | | | 2020/0277376 |
| — | — | Ampsource | U.S. |
| | | | 2019/0367617 |
| — | — | Reyoung (Suzhou) Biology | U.S. |
| | | Science & Technology Co. | 2019/0071501 |
| — | — | Xencor, Inc. | WO |
| | | | 2018/071918 |
| — | — | Crown Bioscience Inc. | WO |
| | | | 2016/014688 |
| — | — | Beijing Hanmi | U.S. |
| | | | 2020/0299412 |
| — | — | Beijing Hanmi | U.S. |
| | | | 2021/0032343 |
| — | — | Beijing Hanmi | U.S. |
| | | | 2019/0367615 |
| — | — | MabQuest | WO |
| | | | 2016/020856 |
| — | — | Fuso Pharma, Hokkaido U. | WO |
| | | | 2018/034226 |
| — | — | Eureka Therapeutics, Inc., | WO |
| | | Sloan-Kettering | 2016/210129 |
| — | — | Sutro Biopharma, Inc. | WO |
| | | | 2016/077397 |
| — | — | Y-Biologics | U.S. |
| | | | 2019/0248900 |
| — | — | Harbour Biomed Ltd. | WO |
| | | | 2017/016497 |
| — | — | Shanghai PharmaExplorer Co. | Liu et al., |
| | | | Sci. Rep. |
| | | | 9(1) (2019) |
| — | — | Bio X Cell | Grasselly et al., |
| | | | Front Immunol |
| | | | 9: 2100 (2018) |
| — | — | Tongji University | Cai et al., |
| | | | Invest. New Drugs |
| | | | 37(5): 799-809 |
| | | | (2019) |
| — | — | Janssen Biotech | U.S. |
| | | | 2018/0355061 |
| — | — | Shanghai Henlius Biotech, | U.S. |
| | | Inc. | 2019/0218295 |
| — | — | Ultrahuman Nine Ltd. | WO |
| | | | 2019/170898 |
| — | — | CytomX | WO |
| | | | 2017/011580 |
| — | — | Nanjing Legend Biotech | WO |
| | | | 2017/133633 |
| — | — | Aduro Biotech | U.S. Pat. No. 10,494,436 |
| — | — | Sutro Biopharm | U.S. Pat. No. 10,822,414 |
| — | — | Versitech Ltd. | U.S. Pat. No. 10,047,137 |
| — | — | STCube & Co., University of | WO |
| | | Texas | 2016/160792 |
| — | — | Tayu Huaxia Biotech | U.S. |
| | | Medivcal Group Co. | 2019/0144543 |
| — | — | Hangzhou Sumgen | U.S. |
| | | Biotechnology | 2019/0016799 |
| — | — | Jiangsu Hengrui Medicine Co, | WO |
| | | Shanghai Hengrui | 2015/085847 |
| | | Pharmaceutical Co | |
| — | — | Salubris (Chengdu) Biotech | WO |
| | | Co. | 2019/201169 |
| — | — | Kadmon Corp. | U.S. Pat. No. 10,407,502 |
| — | — | Augusta University Research | WO |
| | | Institute | 2019/051164 |
| — | — | Abbvie Biotherapeutics Inc. | WO |
| | | | 2018/053106 |
| — | — | Lyvgen Biopharma Holdings | WO |
| | | Ltd. | 2017/087599 |
| — | — | Crescendo Biologies Ltd. | WO |
| | | | 2018/127711 |
| — | — | Zhejiang Teruisi | U.S. |
| | | Pharmaceutics Inc. | 2019/0322747 |
| — | — | Biosion Inc. | WO |
| | | | 2019/204132 |
| — | — | Celgene Corp. | U.S. |
| | | | 2017/0088618 |
| — | — | Asia Biotech Pte. Ltd. | WO |
| | | | 2017/058115 |
| — | — | The Brigham and Women's | U.S. Pat. No. 10,934,352 |
| | | Hospital | |
| — | — | Singapore Immunology | Seidel et al., |
| | | Network (SIgN), Institute of | Front. Oncol. |
| | | Medical Biology, Agency for | 28 (2018) |
| | | Science, Technology and | |
| | | Research (A*STAR) | |
| — | — | Chinese People Liberation | Sun et al., |
| | | Army General Hospital | J. Gastrointest Oncol. |
| | | | 11(6): 1421-1430 |
| | | | (2020) |
| — | — | Immunomedics Inc. | U.S. Pat. No. 10,669,338 |
| — | — | Xiangtan Tenghua Bio | WO |
| | | | 2018/162944 |
| — | — | Beijing Dongfang Biotech | U.S. |
| | | | 2019/0144541 |
| — | — | REMD Biotherapeutics | WO |
| | | | 2018/119474 |
| — | — | Rinat Neuroscience Corp. | U.S. Pat. No. 10,155,037 |
| — | — | Medimmune, Wyeth | WO |
| | | | 2004/056875 |
| — | — | Fudan University | Yuan et al., |
| | | | Invest. New Drugs |
| | | | 39(1): 34-51 |
| | | | (2021) |

### Each of the References Identified in Table 1 is Incorporated Herein by Reference.

In certain embodiments, the PD-1 inhibitor comprises an amino acid sequence having at least about 80%, at least about 85%, at least about 90%, at least about 95%, at least about 98%, at least about 99%, or 100% sequence identity to any of the antibodies identified in Table 1. For example, in some embodiments, the PD-1 inhibitor comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% identity to any of the antibodies identified in Table 1.

In some embodiments, the anti-PD-1 inhibitor is pembrolizumab (Keytruda®). In some embodiments, the PD-1 inhibitor is nivolumab (Opdivo®). In some embodiments, the PD-1 inhibitor is zimberelimab (WBP3055). In some embodiments, PD-1 inhibitor is cetrelimab (JNJ-63723283). In certain embodiments, the PD-1 inhibitor comprises an amino acid sequence having at least about 80%, at least about 85%, at least about 90%, at least about 95%, at least about 98%, at least about 99%, or 100% sequence identity to: a) pembrolizumab (Keytruda®); b) nivolumab (Opdivo®); c) zimberelimab (WBP3055); or d) cetrelimab (JNJ-63723283).

In some embodiments, the anti-PD-1 is selected or derived from those described in U.S. patent application publication numbers U.S. 2019/0270815A1 and U.S. 2019/0225689A1, each of which is incorporated herein by reference.

In some embodiments, the anti-PD-1 is capable of specifically binding to human PD-1 at a Kd value of ≤10⁻¹³M (eg., ≤5×10⁻⁷M.≤2×10⁻⁷M, ≤10⁻⁷M, ≤5×10⁻⁸ M, ≤2×10⁻⁸ M, ≤10⁻⁸M, ≤5×10⁻⁹M, ≤2×10⁻⁹M. ≤10⁻⁹M, ≤5×10⁻¹⁰M, ≤2×10⁻¹⁰M. ≤10⁻¹⁰ M, ≤5×10⁻¹¹M, ≤2×10⁻¹¹M, ≤10⁻¹¹M, ≤5×10⁻¹² M, ≤2×10⁻¹² M, ≤10⁻¹²M, ≤5×10⁻¹³M, ≤2×10⁻¹³ M, and ≤10⁻¹³ M). In some embodiments, the anti-PD-1 is capable of specifically binding to human PD-1 at a Kd value of no more than 108M (eg. no more than ≤9×10⁻⁹M, ≤8×10⁻⁹M. ≤7×10⁻⁹M, ≤6×10⁻⁹M, ≤5×10⁻⁹M, ≤4×10⁻⁹M, ≤3×10⁻⁹M, ≤2×10⁻⁹M, or ≤10⁻⁹M). The Kd value may be measured by plasmon resonance binding assay.

In some embodiments, the anti-PD-1 binds to human PD-1 with an EC₅₀ (i.e. 50% binding concentration) of 0.1 nM-100 nM (e.g. 0.1 nM-50 nM, 0.1 nM-30 nM. 0.1 nM-20 nM, 0.1 nM-10 nM, or 0.1 nM-1 nM). In some embodiments, the anti-PD-1 inhibits the binding of human PD-1 to its ligand at an IC₅₀ of 0.2 nM-100 nM (e.g. 0.2 nM-50 nM, 0.2 nM-30 nM, 0.2 nM-20 nM. 0.2 nM-10 nM, or 1 nM-10 nM), as measured in a competition assay.

In some embodiments, the anti-PD-1 has substantially reduced or depleted effector function. In certain embodiments, the anti-PD-1 has a constant region of IgG4 isotype that has reduced or depleted effector function. In some embodiments, the anti-PD-1 has a fully human IgG sequence, for example an IgG4 sequence. In certain embodiments, the anti-PD-1 does not mediate ADCC and/or CDC.

In certain embodiments, the anti-PD-1 comprises a heavy chain CDR comprising an amino acid sequence selected from the group consisting of: SEQ ID NOs: 310-320, 378-380, and sequences having at least 80%, at least 85%, at least 90%, at least 95%, at least 98%, at least 99%, or 100% sequence identity therewith. In certain embodiments, the anti-PD-1 comprises a light chain CDR comprising an amino acid sequence selected from the group consisting of: SEQ ID NOs: 321-332, 381-383, and sequences having at least 80%, at least 85%, at least 90%, at least 95%, at least 98%, at least 99%, or 100% sequence identity therewith.

In certain embodiments, the anti-PD-1 comprises at least one, two, three, four, five, or six CDRs, each comprising an amino acid sequence individually selected from: SEQ ID NOS: 310-312 and 321-323: SEQ ID NOs: 312-314, 321, 323, and 324; SEQ ID NOs: 310, 312, 314, 321, 324, and 325: SEQ ID NOs: 310, 312, 314, 321, 324, and 332; SEQ ID NOs: 315-317 and 326-328; SEQ ID NOs: 318-320 and 329-331; or SEQ ID NOs: 378-383. In certain embodiments, the anti-PD-1 comprises a heavy chain variable region comprising the amino acid sequences of: SEQ ID NOs: 310, 311, and/or 312; SEQ ID NOs: 312, 313, and/or 314; SEQ ID NOS: 310, 312, and/or 314; SEQ ID NOs: 315, 316, and/or 317; SEQ ID NOs: 318, 319, and/or 320; or SEQ ID NOs: 378, 379, and/or 380. In certain embodiments, the anti-PD-1 comprises a light chain variable region comprising the amino acid sequences of: SEQ ID NOs: 321, 322, and/or 323: SEQ ID NOs: 321, 323, and/or 324; SEQ ID NOs: 321, 324, and/or 325; SEQ ID NOs: 326, 327, and/or 328; SEQ ID NOs: 329, 330, and/or 331; SEQ ID NOS: 321, 324, and/or 332; or SEQ ID NOs: 381, 382, and/or 383. In certain embodiments, the anti-PD-1 comprises: a heavy chain variable region comprising the amino acid sequences of SEQ ID NOs: 310, 311, and/or 312 and a light chain variable region comprising the amino acid sequences of SEQ ID NOs: 321, 322, and/or 323; a heavy chain variable region comprising the amino acid sequences of SEQ ID NOs: 312, 313, and/or 314 and a light chain variable region comprising the amino acid sequences of SEQ ID NOs: 321, 323, and/or 324; a heavy chain variable region comprising the amino acid sequences of SEQ ID NOs: 310, 312, and/or 314 and a light chain variable region comprising the amino acid sequences of SEQ ID NOs: 321, 324, and/or 325; a heavy chain variable region comprising the amino acid sequences of SEQ ID NOs: 315, 316, and/or 317 and a light chain variable region comprising the amino acid sequences of SEQ ID NOs: 326, 327, and/or 328; a heavy chain variable region comprising the amino acid sequences of SEQ ID NOs: 318, 319, and/or 320 and a light chain variable region comprising the amino acid sequences of SEQ ID NOs: 329, 330, and/or 331; a heavy chain variable region comprising the amino acid sequences of SEQ ID NOs: 310, 312, and/or 315 and a light chain variable region comprising the amino acid sequences of SEQ ID NOS: 321, 324, and/or 332; or a heavy chain variable region comprising the amino acid sequences of SEQ ID NOs: 378, 379, and/or 380 and a light chain variable region comprising the amino acid sequences of SEQ ID NOs: 381, 382, and/or 383. In each of the aforementioned embodiments, the CDRs may instead comprise amino acid sequences having at least 80%, at least 85%, at least 90%, at least 95%, at least 98%, or at least 99% sequence identity with the recited sequences.

In certain embodiments, the fusion protein comprises: three heavy chain CDRs, the CDRs comprising, respectively, amino acid sequences of SEQ ID NOs: 378, 379, and 380, or sequences having at least 80%, at least 85%, at least 90%, at least 95%, at least 98%, at least 99%, or 100% sequence identity therewith; and three light chain CDRs, the CDRs comprising, respectively, amino acid sequences of SEQ ID NOs: 381, 382, and 383, or sequences having at least 80%, at least 85%, at least 90%, at least 95%, at least 98%, at least 99%, or 100% sequence identity therewith.

In certain embodiments, the fusion protein comprises: three heavy chain CDRs, the CDRs comprising, respectively, amino acid sequences of SEQ ID NOs: 378, 379, and 380; and three light chain CDRs, the CDRs comprising, respectively, amino acid sequences of SEQ ID NOs: 381, 382, and 383.

In certain embodiments, the anti-PD-1 comprises a heavy chain variable region comprising an amino acid sequence of one of SEQ ID NOs: 333-337, 384, or a sequence having at least 80%, at least 85%, at least 90%, at least 95%, at least 98%, at least 99%, or 100% sequence identity with such a sequence. In certain embodiments, the anti-PD-1 comprises a light chain variable region comprising an amino acid sequence of one of SEQ ID NOs: 338-343, 385, or a sequence having at least 80%, at least 85%, at least 90%, at least 95%, at least 98%, at least 99%, or 100% sequence identity with such a sequence.

In certain embodiments, the anti-PD-1 comprises: a heavy chain variable region comprising an amino acid sequence of SEQ ID NO: 333 and a light chain variable region comprising an amino acid sequence of SEQ ID NO: 338; a heavy chain variable region comprising an amino acid sequence of SEQ ID NO: 334 and a light chain variable region comprising an amino acid sequence of SEQ ID NO: 339: a heavy chain variable region comprising an amino acid sequence of SEQ ID NO: 335 and a light chain variable region comprising an amino acid sequence of SEQ ID NO: 340; a heavy chain variable region comprising an amino acid sequence of SEQ ID NO: 336 and a light chain variable region comprising an amino acid sequence of SEQ ID NO: 341; a heavy chain variable region comprising an amino acid sequence of SEQ ID NO: 337 and a light chain variable region comprising an amino acid sequence of SEQ ID NO: 342; a heavy chain variable region comprising an amino acid sequence of SEQ ID NO: 335 and a light chain variable region comprising an amino acid sequence of SEQ ID NO: 343; or a heavy chain variable region comprising an amino acid sequence of SEQ ID NO: 384 and a light chain variable region comprising an amino acid sequence of SEQ ID NO: 385. In each of the aforementioned embodiments, the heavy and light chain variable regions may instead comprise amino acid sequences having at least 80%, at least 85%, at least 90%, at least 95%, at least 98%, or at least 99% sequence identity with the recited sequences.

In certain embodiments, the anti-PD-1 comprises a heavy chain comprising an amino acid sequence of SEQ ID NO: 386, or a sequence having at least 80%, at least 85%, at least 90%, at least 95%, at least 98%, at least 99%, or 100% sequence identity therewith, and a light chain comprising an amino acid sequence of SEQ ID NO: 387, or a sequence having at least 80%, at least 85%, at least 90%, at least 95%, at least 98%, at least 99%, or 100% sequence identity therewith. In certain embodiments, the anti-PD-1 comprises a heavy chain comprising and amino acid sequence of SEQ ID NO: 386 and a light chain comprising an amino acid sequence of SEQ ID NO: 387.

In certain embodiments, the anti-PD-1 is a fully human monoclonal antibody that comprises a human constant region of the lgG4 isotype and: a heavy chain variable region comprising the amino acid sequence of SEQ ID NO: 333 and a light chain variable region comprising the amino acid sequence of SEQ ID NO: 338; a heavy chain variable region comprising the amino acid sequence of SEQ ID NO: 334 and a light chain variable region comprising the amino acid sequence of SEQ ID NO: 339; a heavy chain variable region comprising the amino acid sequence of SEQ ID NO: 335 and a light chain variable region comprising the amino acid sequence of SEQ ID NO: 340; a heavy chain variable region comprising the amino acid sequence of SEQ ID NO: 335 and a light chain variable region comprising the amino acid sequence of SEQ ID NO: 343: a heavy chain variable region comprising the amino acid sequence of SEQ ID NO: 336 and a light chain variable region comprising the amino acid sequence of SEQ ID NO: 341; a heavy chain variable region comprising the amino acid sequence of SEQ ID NO: 337 and a light chain variable region comprising the amino acid sequence of SEQ ID NO: 342; or a heavy chain variable region comprising an amino acid sequence of SEQ ID NO: 384 and a light chain variable region comprising an amino acid sequence of SEQ ID NO: 385. In each of the aforementioned embodiments, the heavy and light chain variable regions may instead comprise amino acid sequences having at least 80%, at least 85%, at least 90%, at least 95%, at least 98%, or at least 99% sequence identity with the recited sequences.

In some embodiments, the anti-PD-1 competes for the same epitope as any of the aforementioned antibodies. For example, the anti-PD-1 may block the binding of the antibody to human PD-1 at an IC₅₀ value (i.e. 50% inhibition concentration) of below 10⁻⁶ M, below 10⁻⁷M, below 10^{-7.5} M, below 10⁻⁸ M, below 10^{-3.5} M, below 10⁻⁹M, or below 10⁻¹⁰ M. The IC₅₀ values are determined based on a competition assay such as ELISA assays, radioligand competition binding assays, and FACS analysis.

In some embodiments, the anti-PD-1 binds to a buroan PD-1 comprising the amino acid sequence in Table 26, and reproduced below:

(SEQ ID NO: 388)
FLDSPDRPWNPPTFSPALLVVTEGDNATFTCSFSNTSESFVLNWYRMS
PSNQTDKLAAFPEDRSQPGQDCRFRVTQLPNGRDFHMSVVRARRNDSG
TYLCGAISLAPKAQIKESLRAELRVTERRAEVPTAHPSPSPRPAGQFQ
TLVVGVVGGLLGSLVLLVWVLAVICSRAARGTIGARRTGQPLKEDPSA
VPVFSVDYGELDFQWREKTPEPPVPCVPEQTEYATIVFPSGMGTSSPA
RRGSADGPRSAQPLRPEDGHCSWPL

In some embodiments, the anti-PD-1 binds to an epitope of human PD-1 comprising at least one of the following residues of SEQ ID NO: 388: V41, P60, D62, LI05, A106, P107, K108, A109 and Q110, shown in emphasis below.

(SEQ ID NO: 388)
FLDSPDRPWNPPTFSPALLVVTEGDNATFTCSFSNTSESFVLNWYRMS
PSNQTDKLAAFPEDRSQPGQDCRFRVTQLPNGRDFHMSVVRARRNDSG
TYLCGAISLAPKAQIKESLRAELRVTERRAEVPTAHPSPSPRPAGQFQ
TLVVGVVGGLLGSLVLLVWVLAVICSRAARGTIGARRTGQPLKEDPSA
VPVFSVDYGELDFQWREKTPEPPVPCVPEQTEYATIVFPSGMGTSSPA
RRGSADGPRSAQPLRPEDGHCSWPL

In some embodiments, the anti-PD-1 is capable of blocking the binding of human PD-1 to its ligand and thereby: inducing production of IL-2 in CD4+ T cells; inducing production of IFNγ in CD4+ T cells; inducing proliferation of CD4+ T cells; and/or reversing the T cells' suppressive function.

In some embodiments, the anti-PD-1 is a monoclonal antibody, a polyclonal antibody, a fully human antibody, a humanized antibody, a chimeric antibody, a recombinant antibody, a bispecific antibody, a labeled antibody, a bivalent antibody, or an anti-idiotypic antibody. In some embodiments, the anti-PD-1 is a fully human monoclonal antibody, optionally produced by a transgenic mouse or rat, for example, a transgenic rat having inactivated endogenous expression of rat immunoglobulin genes and carrying recombinant human immunoglobulin loci having J-locus deletion and a C-kappa mutation.

In some embodiments, the anti-PD-1 is a camelized single domain antibody, a diabody, a scFv, an scFv dimer, a BsFv, a dsFv, a (dsFv)₂, a dsFv-dsFv′, an Fv fragment, a Fab, a Fab′, a F(ab′)₂, a ds diabody, a nanobody, a domain antibody, or a bivalent domain antibody.

In some embodiments, the anti-PD-1 further comprises an immunoglobulin constant region.

In some embodiments, the anti-PD-1 further comprises a conjugate. The conjugate may, for example, be a detectable label, a pharmacokinetic modifying moiety, or a purification moiety.

### Tumor Antigen-Binding Antibody

In certain embodiments, the antibody, or fragment or variant thereof, of the first component binds to a tumor antigen expressed on the surface of a tumor cell.

The antibody, or fragment or variant thereof, can be an antibody, antibody fragment, scFv, Fc-containing polypeptide, fusion antibody, polypeptide, peptide, aptamer, ligand, nucleic acid, or any combination thereof. In one embodiment, the antibody, or fragment or variant thereof, can bind to a molecule present in a cell or tissue. In one aspect, the targeting moiety can bind a molecule in a diseased cell or tissue, such as a cancer cell or tumor. In another aspect, the antibody, or fragment or variant thereof, can bind a normal cell or tissue, such as an immune cell. In another aspect, the antibody, or fragment or variant thereof, can bind a cellular or extracellular molecule that modulates the immune response. In another aspect, the antibody, or fragment or variant thereof, binds a growth factor receptor, growth factor, cytokine receptor, cytokine, or cell surface molecule. In one embodiment, the antibody, or fragment or variant thereof, binds to an epitope on CD19, BCMA, CD44, a-Folate receptor, CAIX, CD30, ROR1, CEA, EGP-2, EGP-40, HER2, HER3, Folate-binding Protein, GD2, GD3, IL-13R-a2, KDR, EDB-F, mesothelin, CD22, EGFR, Folate receptor α, MUCI, MUC4, MUC16, MAGE-A1, h5T4, PSMA, TAG-72, EGFR, CD20, EGFRVIII, CD123 or VEGF-R2. In one embodiment the antibody, or fragment or variant thereof, binds to an epitope on MUCI, MUC2, MUC3A, MUC3B, MUC4, MUC5AC, MUC5B, MUC6, MUC7, MUC8, MUC12, MUC13, MUC15, MUC16, MUC 17, MUC19, or MUC20. In one embodiment the antibody, or fragment or variant thereof, binds to an epitope on MUC1. In one embodiment, the antibody, or fragment or variant thereof, binds to an epitope on MUC16. In one embodiment, the antibody, or fragment or variant thereof, includes an antibody, antibody fragment, scFv, or Fc-containing polypeptide. In some cases, the antibody is immunoglobulin G (IgG) antibody. In some embodiments, the IgG is IgG 1, IgG2, IgG3, or lgG4. In one embodiment, the IgG is IgG1. In other cases, the fragment of the antibody is a Fab, (Fab)₂, (Fab′)₂, Fv, (Fv)₂, or scFv of the antibody. In another case, the antibody comprises a variable region of heavy chain (V_{H}) and a variable region of light chain (V_{L}). In some embodiments, the antibody further comprises fragment crystallizable region (Fc). In yet another case, the Fc is human Fc1, Fc2, Fc3, Fc4, or a fragment thereof. In some embodiments, the Fe further comprises one or more mutations. In some embodiments, the antibody comprises the scFv and the Fc fragment.

### MUCIN I, Cell Surface Associated Protein (MUCI)

MUCI (mucin 1, cell surface associated protein) is a mucin-type glycoprotein that is expressed on the apical borders of normal secretory epithelial cells. MUCI forms a heterodimer following synthesis as a single polypeptide and cleavage of the precursor into two subunits in the endoplasmic reticulum. MUCI N-terminal (MUCI N-ter, MUCI-N) subunit contains variable numbers of 20 amino acid tandem repeats that are imperfect with highly conserved variations and are modified by O-linked glycans. MUC 1-N is tethered to the cell surface by dimerization with the about 23 kDa C-terminal subunit (MUCI C-ter, MUCI-C), which includes a 58 amino acid extracellular region, a 28 amino acid transmembrane domain and a 72-amino acid cytoplasmic domain (CD). The tumor targeting moiety as described herein includes antibodies that bind to the 58 amino acid portion of the MUCI-C/ECD. Examples of MUCI antibodies are described in U.S. Pat. No. 10,059,775, U.S. Patent Application Publication No. 20180036441, U.S. Pat. No. 9,932,407, U.S. Patent Application Publication Nos. 20180112007, 20180222998, WO2018071583, U.S. Pat. No. 7,202,346, all of which are herein incorporated by reference in their entireties.

In one embodiment, the targeting moiety is a tumor targeting moiety that binds to an epitope on MUC1. In some embodiments, the anti-MUC1 antibody fragment is a Fab, Fab2, (Fab′)₂, Fv, (Fv)₂, scFv, scFv-Fc, Fc, diabody, triabody, or mini body of the anti-MUC1. In some embodiments, the anti-MUC1 antibody fragment is a single-domain antibody of the anti-MUCI antibody. In some embodiments, the single-domain antibody is a V_{NAR} or V_{H}H fragment of the anti-MUC1 antibody.

In some embodiments, the variable region of heavy chain (V_{H}) of an anti-MUC1 antibody or a fragment/variant thereof comprises one or more polypeptide sequences as shown in any one of SEQ ID NOs: 444-448. In some embodiments, the variable region of heavy chain (V_{H}) of an anti-MUC1 antibody or a fragment/variant thereof comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 444-448.

In some embodiments, the variable region of light chain (V_{L}) of an anti-MUC1 antibody or a fragment/variant thereof comprises one or more polypeptide sequences as shown in any one of SEQ ID NOs: 449-453. In some embodiments, the variable region of light chain (V_{L}) of an anti-MUC1 antibody or a fragment/variant thereof comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 449-453.

In some embodiments, the anti-MUC1 antibody, or a fragment/variant thereof, comprises a variable region of light chain (V_{L}) comprising a sequence that is at least 80% identical to of any one of SEQ ID NOs: 449-453 and a heavy chain (V_{H}) comprising a sequence that is at least 80% identical to SEQ ID NO: 444.

### MUCIN 16, Cell Surface Associated Protein (MUC16)

MUC16 (also known as CA-125 (cancer antigen 125, carcinoma antigen 125, or carbohydrate antigen 125) or mucin 16), is a member of the mucin family of glycoproteins. MUC 16 has been shown to play a role in advancing tumorigenesis and tumor proliferation by several different mechanisms.

MUC16 is a large carbohydrate antigen, also known as CA-125. MUC16 is encoded by the MUC16 gene located on human chromosome 19. MUC16 is a highly glycosylated multidomain type I transmembrane protein comprising 3 domains. The C-terminal domain contains multiple extracellular SEA (sea urchin sperm protein, enterokinase, and agrin) modules that have an autoproteolytic activity. SEA harbors two proteolytic sites proximal to the transmembrane (TM) domain. A large cleaved domain termed CA-125 is released into circulation at acidic pH. CA-125 is commonly used as disease biomarker for ovarian cancer. The highly conserved truncated extracellular membrane tethered protein domain called MUC16ecto domain. A MUC16 antibody was identified that specifically bound the ectodomain of MUC16 that is retained on the tumor cell surface. “Overexpression of MUC16” by a cell of interest (such as a cancer cell) refers to a higher level of MUC16 protein and/or mRNA that is expressed by the cell of interest compared to a control cell (such as a non-cancerous cell, normal cell, etc.).

In one embodiment the targeting moiety is a tumor targeting moiety that binds to an epitope on MUC16. In some embodiments, the anti-MUC16 antibody fragment is a Fab, Fab2, (Fab′)₂, Fv, (Fv)₂, scFv, scFv-Fc, Fc, diabody, triabody, or mini body of the anti-MUC16. In some embodiments, the anti-MUC16 antibody fragment is a single-domain antibody of the anti-MUC16 antibody. In some embodiments, the single-domain antibody is a V_{NAR} Or V_{H}H fragment of the anti-MUC16 antibody.

In some embodiments, the variable region of heavy chain (V_{H}) of an anti-MUC16 antibody or a fragment/variant thereof comprises one or more polypeptide sequences as shown in any one of SEQ ID NOs: 390-403. In some embodiments, the variable region of heavy chain (V_{H}) of an anti-MUC 16 antibody or a fragment/variant thereof comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOS: 390-403.

In some embodiments, the variable region of light chain (V_{L}) of an anti-MUC16 antibody or a fragment/variant thereof comprises one or more polypeptide sequences as shown in any one of SEQ ID NOs: 417-428. In some embodiments, the variable region of light chain (V_{L}) of an anti-MUC16 antibody or a fragment/variant thereof comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 417-428.

### Cytokine Trap

Cytokines have an impact on many biological processes. Inhibiting cytokines can have clinical benefits for example, in cancer. Several cytokines have been shown to be causative agents in a variety of diseases. Such cytokines, include but are not limited to IL-1, IL-4, IL-6, TNF-α, TGF-beta and its various isoforms. The term “cytokine trap” as used herein refers to blockers or neutralizers of cytokine action. Examples of such cytokine traps can include but are not limited to extracellular domains of the cytokine receptors, antibodies that bind to cytokines, and peptides that bind to the cytokines (e.g. inhibitory peptides). In some embodiments, the cytokine is TGF-β. In some embodiments, the cytokine is TGF-β1. In some embodiments, the cytokine is TGF-β3. In some embodiments, the cytokine is TGF-β1 and TGF-β3. In further embodiments, cytokine traps that target TGF-β (e.g. TGF-β trap) can include the extracellular domain of TGF-βRII or its variants thereof (for example, SEQ ID NOs: 141 and 142), anti-TGF-β antibodies and inhibitory peptides of TGF-β1, TGF-β2 and/or TGF-β3.

### Transforming Growth Factor-β

Transforming growth factor-β (TGF-β) is a multifunctional set of peptides that can control proliferation, differentiation, and other functions in many cell types. TGF-β can act synergistically with TGF-α in inducing transformation. It also can act as a negative autocrine growth factor. Dysregulation of TGF-β activation and signaling can result in apoptosis. Many cells can synthesize TGF-β and almost all of them have specific receptors for this peptide. TGF-β1, TGF-β2, and TGF-β3 all can function through the same receptor signaling systems. TGF-β1 can play an important role in controlling the immune system, and can show different activities on different types of cell, or cells at different developmental stages. Most immune cells (or leukocytes) can secrete TGF-β1. TGF-β1 is a peptide of 112 amino acid residues derived by proteolytic cleavage from the C-terminal of a precursor protein. TGF-β, a small secreted polypeptide, can signal through TGFβRII, which can recruit and phosphorylate the type I dimeric receptor (TGFβRI). TGFβRI can phosphorylate and activate SMADs which can be transcription factors regulating genes involved in cell proliferation, differentiation, apoptosis and growth. Many advanced stage cancers are known to over-express both the TGF-β and TGFβR promoting aggressive tumor formation. Inhibiting the TGFB signaling pathway can be a key therapeutic strategy in treating cancer.

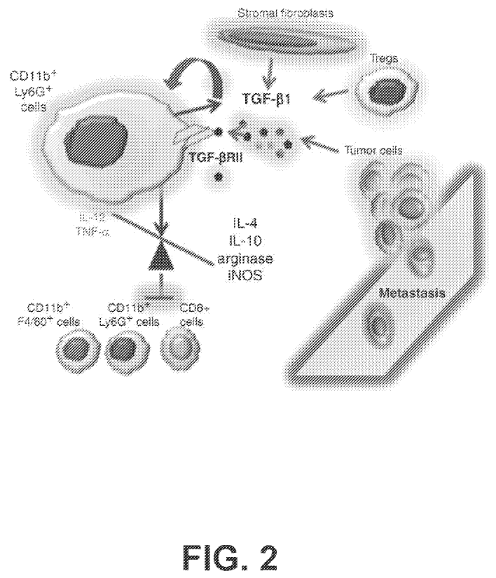

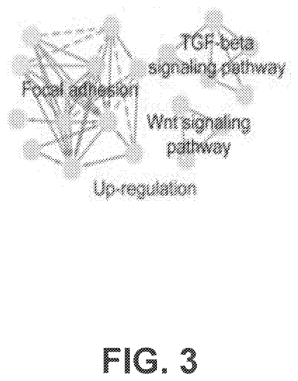

Some T cells (e.g., regulatory T cells) can release TGF-β1 to inhibit the actions of other T cells. IL-1 and IL-2 dependent proliferation of activated T cells, and the activation of quiescent helper T cells and cytotoxic T cells can be prevented by the activity of TGF-β1. Similarly, TGF-β1 can inhibit the secretion and activity of many other cytokines including, but not limited to, interferon-γ, tumor necrosis factor alpha (TNF-α) and various interleukins. It can also decrease the expression levels of cytokine receptors, such as the IL-2 receptor, to down-regulate the activity of immune cells. However, TGF-β1 can also increase the expression of certain cytokines in T cells and can promote their proliferation, particularly if the cells are immature (FIG. 2).

TGF-β1 can have similar effects on B cells that vary according to the differentiation state of the cell. It can inhibit proliferation and can stimulate apoptosis of B cells, and can play a role in controlling the expression of antibody, transferrin, and MHC class II proteins on immature and mature B cells.

The effects of TGF-β1 on macrophages and monocytes can be predominantly suppressive; this cytokine can inhibit the proliferation of these cells and can prevent their production of reactive oxygen (e.g., superoxide (02)) and nitrogen (e.g., nitric oxide (NO)) intermediates. However, as with other cell types, TGF-β1 can also have the opposite effect on cells of myeloid origin. For example, TGF-β1 can act as a chemoattractant, directing an immune response to some pathogens; macrophages and monocytes can respond to low levels of TGF-β1 in a chemotactic manner. Furthermore, the expression of monocytic cytokines (including IL-la, IL-1B, and TNF-α), and phagocytic killing by macrophages can be increased by the action of TGF-β1 (FIG. 2).

Transforming Growth Factor-Beta III (TGF-β3), a subset of a cytokine family, is responsible for a plethora of functions including cellular proliferation, embryogenesis, immune system regulation, and differentiation.

### Transforming Growth Factor-β Receptor Type II (TGFβRII)

TGF-β receptors (TGFβR) are single pass serine/threonine kinase receptors. They can exist in several different isoforms that can be homodimeric or heterodimeric. The number of characterized ligands in the TGF-β superfamily can far exceed the number of known receptors, suggesting the promiscuity between the ligand and receptor interactions. Three TGF-β superfamily receptors (TGFβR) specific for TGF-β can be distinguished by their structural and functional properties. TGFβRI (ALK5) and TGFβRII can have similar ligand-binding affinities and can be distinguished from each other only by peptide mapping. Both TGFβRI and TGFβRII can have a high affinity for TGF-β1 and low affinity for TGF-β2. TGFβRIII (β-glycan) can have a high affinity for both homodimeric TGF-β1 and TGF-β2 and in addition the heterodimer TGF-β1,2. The TGFB receptors can also bind to TGF-β3. By “TGFβRII” or “TGFB Receptor II” is meant a polypeptide having the wild-type human TGFβ Receptor Type 2 Isoform A sequence (e.g., the amino acid sequence of NCBI Reference Sequence (RefSeq) Accession No. NP_001020018 (SEQ ID NO: 289), or a polypeptide having the wild-type human TGFB Receptor Type 2 Isoform B sequence (e.g., the amino acid sequence of NCBI RefSeq Accession No. NP_003233 (SEQ ID NO: 290) or having a sequence substantially identical the amino acid sequence of SEQ ID NO: 289 or of SEQ ID NO: 290. The TGFβRII may retain at least 0.1%, 0.5%, 1%, 5%, 10%, 25%, 35%, 50%, 75%, 90%, 95%, or 99% of the TGFB-binding activity of the wild-type sequence. The polypeptide of expressed TGFβRII lacks the signal sequence.

TGF-β1 can reduce the efficacy of the MHC II in astrocytes and dendritic cells, which in turn can decrease the activation of appropriate helper T cell populations. TGF-β1 can promote tumor growth as cancer progresses and in some embodiments, does not suppress inflammatory cells responses but can promote regulatory T cell function. TGF-β1 can be produced by tumor cells, tumor-associated fibroblast cells, regulatory T cells and immature myeloid cells. TGF-β1 can inhibit T cell priming and promote an exhausted phenotype. TGF-β1 can suppress the anti-tumor activity of innate immune cell populations including natural killer cells, macrophages and dendritic cells. TGF-β receptor II can be upregulated by tumor-associated myeloid cells and can promote metastasis.

### TGF-β Trap Fusion Proteins, or Fragments or Variants Thereof

Provided herein is a fusion protein, or a fragment or a variant thereof, comprising a cytokine trap that can neutralize the cytokine (for instance, TGF-β). In certain cases, a cytokine trap can be a TGF-β trap (also referred to as TGF-βRII or fragment or variant thereof) that comprises SEQ ID NO. 142. Examples of TGF-β trap can include, but are not limited to, extracellular domain (ECD) of the receptor (e.g., TGFβRII), or a functional variant or derivative thereof, TGF-β inhibitory peptides (for instance, SEQ ID NOs: 468-507 and 263-267), or an anti-TGF-β antibodies. In some embodiments, the anti-TGF-β antibody comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence selected from any one of SEQ ID NOs: 166, 168, 169, 171, 173, 175, or 177. In some embodiments, the anti-TGF-β antibody comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence selected from any one of SEQ ID NOs: 165, 167, 170, 172, 174, 176, or 178. In certain embodiments, a TGF-β trap can specifically bind or have high affinity for TGF-β1 or TGF-β2 or TGF-β3. In other embodiments, a TGF-β trap can specifically bind or have high affinity for TGF-β1, TGF-β2 and TGF-β3. In other embodiments, a TGF-β trap can specifically bind or have high affinity for TGF-β1 and TGF-β3. In further embodiments, a TGF-β trap can have low affinity for or not bind TGF-β2.

The fusion protein (or fragment or variant thereof) provided herein can elicit a synergistic anti-tumor effect due to the simultaneous blockade of the interaction between e.g., PD-L1, MUC1, and MUC16 on tumor cells and PD-1, MUCI, and MUC16 on immune cells, and the neutralization of e.g., TGF-β in the tumor microenvironment. Without being bound by theory, this effect is obtained from simultaneous blocking the two major immune escape mechanisms and the targeted depletion of the TGF-β in the tumor microenvironment by a single molecular entity. This depletion can be achieved by one or more of the following: (1) anti-PD-1, anti-MUC1, or anti-MUC16 targeting of tumor cells; (2) binding of the TGF-β in the tumor microenvironment by the TGF-β trap (e.g., TGFbRII); and/or (3) destruction of the bound TGF-β through the PD-L1 receptor mediated endocytosis. The fusion protein (or fragment or variant thereof) provided herein (e.g., PD-1 inhibitor or antibody, anti-MUC16 antibody, or anti-MUC1 antibody fused to a cytokine trap such as a TGF-β trap) can also promote natural killer cell-mediated killing of tumor cells.

### TGFB-RII Fusion

In some embodiments, the cytokine trap (e.g., TGF-β trap) is a cytokine receptor (e.g., TGFβRII). In some embodiments, the cytokine receptor sequence in a fusion protein described herein comprises an extracellular domain (ECD) of the receptor (e.g., TGFβRII), or a functional variant or derivative thereof. In some embodiments, the extracellular domain (ECD) of TGFβRII comprises a polypeptide sequence as shown in SEQ ID NO: 14. In some embodiments, the cytokine receptor sequence in a fusion protein, or a fragment or a variant thereof, described herein comprises a polypeptide sequence having at least 50%, at least 55%, at least 60%, at least 65%, at least 70%, at least 75%, at least 80%, at least 85%, at least 90%, at least 95%, at least 99%, at least 99.5% or 100% identity with the polypeptide sequence of SEQ ID NO: 14. In some embodiments, the cytokine receptor sequence in a fusion protein described herein comprises an extracellular domain (ECD) of the receptor (e.g., TGFβRII), or a functional variant or derivative thereof. In some embodiments, the extracellular domain (ECD) of TGFβRII comprises a polypeptide sequence as shown in SEQ ID NO: 141. In some embodiments, the cytokine receptor sequence in a fusion protein, or a fragment or a variant thereof, described herein comprises a polypeptide sequence having at least 50%, at least 55%, at least 60%, at least 65%, at least 70%, at least 75%, at least 80%, at least 85%, at least 90%, at least 95%, at least 99%, at least 99.5% or 100% identity with the polypeptide sequence of SEQ ID NO: 141. In some embodiments, the cytokine receptor sequence in a fusion protein described herein comprises an extracellular domain (ECD) of the receptor (e.g., TGFβRII), or a fragment or variant thereof. In some embodiments, the extracellular domain (ECD) of TGFβRII comprises a polypeptide sequence as shown in SEQ ID NO: 142. In some embodiments, the cytokine receptor sequence in a fusion protein, or a fragment or a variant thereof described herein comprises a polypeptide sequence having at least 50%, at least 55%, at least 60%, at least 65%, at least 70%, at least 75%, at least 80%, at least 85%, at least 90%, at least 95%, at least 99%, at least 99.5% or 100% identity with the polypeptide sequence of SEQ ID NO: 142. In some embodiments, the cytokine receptor sequence in a fusion protein, or a fragment or a variant thereof described herein binds TGF-β1 and/or TGF-β3 but does not bind TGF-β2. In certain embodiments, the cytokine receptor sequence in a fusion protein or, a fragment or a variant thereof described herein only binds TGF-β1. In certain embodiments, the cytokine receptor sequence in a fusion protein, or a fragment or a variant thereof described herein only binds TGF-β3. In certain embodiments, the cytokine receptor sequence in a fusion protein, or a fragment or variant thereof, described herein only binds TGF-β1 and/or TGF-β3 but has low to no affinity for TGF-β2.

In some embodiments, the antibody of the first component is fused to TGFβRII or a fragment thereof (e.g., ECD of TGFβRII). In some embodiments, the antibody moiety is fused to TGFβRII or a fragment thereof (e.g., ECD of TGFβRII) via a linker. In some embodiments, the antibody moiety is fused to at least one extracellular domain of TGFβRII. In some embodiments, the antibody moiety is fused to at least one extracellular domain of TGFβRII via a linker.

In some embodiments, the antibody fragment or variant of the first component is a Fab, Fab2, (Fab′)₂, Fv, (Fv)₂, scFv, scFv-Fc, Fc, diabody, triabody, or minibody of the antibody of the first component. In some embodiments, the antibody fragment is a single-domain antibody of the antibody. In some embodiments, the single-domain antibody is a V_{NAR} or V_{H}H fragment of the antibody.

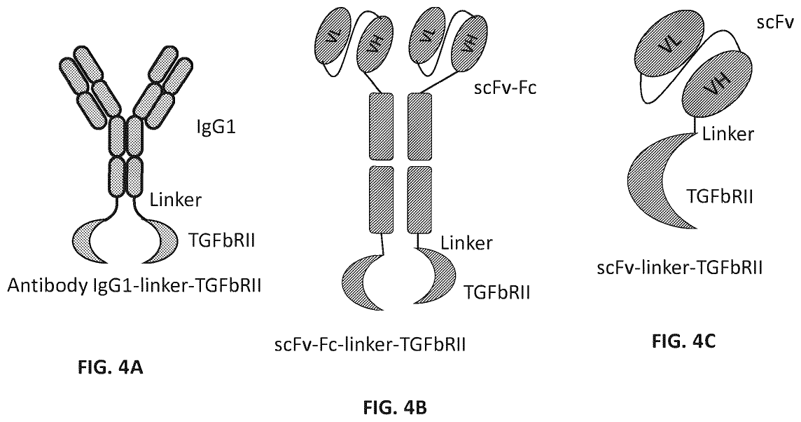

Non-limiting exemplary fusion proteins are illustrated in FIGS. 4A-4C.

In some embodiments, the TGF-β trap (e.g., TGFβRII) is fused to a variable region of heavy chain (V_{H}) of the antibody of the first component or a fragment or variant thereof. In other embodiments, the TGF-β trap is fused to IgG of the antibody of the first component (for example, FIG. 4A). In certain aspects, the IgG is IgG1, IgG2, IgG3, or IgG4. In an embodiment, the IgG is IgG4. In another embodiment, the IgG4 comprises a sequence having at least 80% sequence identity to SEQ ID NO 146 (wild type), SEQ ID NO: 291, SEQ ID NO: 292 or SEQ ID NO: 147 (S108P). In some embodiments, the TGF-β trap (e.g., TGFβRII) is fused to a variable region of heavy chain (V_{H}) of the antibody of the first component, or a fragment or variant thereof, via a linker. In some embodiments, the TGF-β trap (e.g., TGFβRII) is fused to a constant region of the V_{H} of the antibody, or a fragment or variant thereof, via a linker. Examples of V_{H} sequence of an anti-PD-1 antibody or fragment or variant thereof include but are not limited to SEQ ID NOs: 1-7 and 149-164. Examples of V_{L} sequence of an anti-PD-1 antibody or fragment or variant thereof include but are not limited to SEQ ID NOS: 8-13 and 148. Examples of V_{H} sequence of a MUCI targeting antibody or fragment or variant thereof include but are not limited to SEQ ID NOs: 444-448. Examples of V_{L} sequence of a MUCI targeting antibody or fragment or variant thereof include but are not limited to SEQ ID NOs: 449-453. Examples of V_{H} sequence of a MUC16 targeting antibody or fragment or variant thereof include but are not limited to SEQ ID NOs: 390-403. Examples of V_{L}, sequence of a MUC16 targeting antibody or fragment or variant thereof include but are not limited to SEQ ID NOs: 417-428. In some embodiments, the TGF-β trap (e.g., TGFβRII) is fused a variable region of light chain (V_{L}) of the antibody, or a fragment or variant thereof. In some embodiments, the TGF-β trap (e.g., TGFβRII) is fused to a constant region of the V_{L} of the antibody, or a fragment or variant thereof, via a linker. In some embodiments, the TGF-β trap (e.g., TGFβRII) is fused a variable region of light chain (V_{L}) of the antibody, or a fragment or variant thereof, via a linker. In one aspect, the TGF-β trap (e.g., TGFβRII) is fused to either the N- or C-terminus of the V_{L} or V_{H} chain, or a fragment or variant thereof, via a linker.

The term “anti-PD-1 (VL/VH)-TGFβRII” or “anti-PD-1 (VH/VL)-TGFβRII” is used interchangeably and denotes the specific VL or VH used in the fusion protein. In some embodiments, the terms “anti-PD-1 (VL/VH)-TGFβRII” or “anti-PD-1 (VH/VL)-TGFβRII” refers to TGF-β trap (e.g., TGFβRII) fused to constant region of the heavy chain of anti-PD-1 or alternatively, refers to TGF-β trap (e.g., TGFβRII) fused to constant region of the light chain of anti-PD-1.

In some embodiments, the TGF-β trap (e.g., TGFβRII) is fused to a Fab of the antibody of the first component, or a fragment or variant thereof. In some embodiments, the TGF-β trap (e.g., TGFβRII) is fused to a Fab of the antibody, or a fragment or variant thereof, via a linker. In some embodiments, the TGF-β trap (e.g., TGFβRII) is fused to a Fab2 of the antibody, or a fragment or variant thereof. In some embodiments, the TGF-β trap (e.g., TGFβRII) is fused to a Fab2 of the antibody, or a fragment or variant thereof, via a linker. In some embodiments, the TGF-β trap (e.g., TGFβRII) is fused to a (Fab′)₂ of the antibody, or a fragment or variant thereof. In some embodiments, the TGF-β trap (e.g., TGFβRII) is fused to a (Fab′)₂ of the antibody, or a fragment or variant thereof, via a linker. In one aspect, the TGF-β trap (e.g., TGFβRII) is fused to either the N- or C-terminus of the Fab or (Fab′)₂ of the antibody, or a fragment or variant thereof, via a linker.

In some embodiments, the TGF-β trap (e.g., TGFβRII) is fused to an Fv of the antibody of the first component, or a fragment or variant thereof. In some embodiments, the TGF-β trap (e.g., TGFβRII) is fused to a Fv of the antibody, or a fragment or variant thereof, via a linker. In some embodiments, the TGF-β trap (e.g., TGFβRII) is fused to a (Fv)₂ of the antibody, or a fragment or variant thereof. In some embodiments, the TGF-β trap (e.g., TGFβRII) is fused to a (Fv)₂ of the antibody, or a fragment or variant thereof, via a linker. In one aspect, the TGF-β trap (e.g., TGFβRII) is fused to either the N- or C-terminus of the Fv or (Fv)₂ of the antibody, or a fragment or variant thereof, via a linker.

In some embodiments, the TGF-β trap (e.g., TGFβRII) is fused to a scFv of the antibody of the first component, or a fragment or variant thereof. In some embodiments, the TGF-β trap (e.g., TGFβRII) is fused to a scFv of the antibody, or a fragment or variant thereof, via a linker. In some embodiments, the TGF-β trap (e.g., TGFβRII) is fused to a scFv-Fc of the antibody, or a fragment or variant thereof. In some embodiments, the TGF-β trap (e.g., TGFβRII) is fused to a scFv-Fc of the antibody, or a fragment or variant thereof, via a linker. In one aspect, the TGF-β trap (e.g., TGFβRII) is fused to either the N- or C-terminus of the scFv or scFv-Fc of the antibody, or a fragment or variant thereof, via a linker.

In some embodiments, the TGF-β trap (e.g., TGFβRII) is fused to a Fc of the antibody of the first component, or a fragment or variant thereof. In some embodiments, the TGF-β trap (e.g., TGFβRII) is fused to a Fc of the antibody, or a fragment or variant thereof, via a linker. In some embodiments, the TGF-β trap (e.g., TGFβRII) is linked to a C-terminus Fc of the antibody, or a fragment or variant thereof. In some embodiments, the TGF-β trap (e.g., TGFβRII) is linked to a C-terminus Fc of the antibody, or a fragment or variant thereof, via a linker. In some embodiments, the TGF-β trap (e.g., TGFβRII) is linked to an N-terminus Fc of the antibody, or a fragment or variant thereof. In some embodiments, the TGF-β trap (e.g., TGFβRII) is linked to an N-terminus Fc of the antibody, or a fragment or variant thereof, via a linker.

In some embodiments, the TGF-β trap (e.g., TGFβRII) is fused to a diabody of the antibody of the first component, or a fragment or variant thereof. In some embodiments, the TGF-β trap (e.g., TGFβRII) is fused to a diabody of the antibody, or a fragment or variant thereof, via a linker. In some embodiments, the TGF-β trap (e.g., TGFβRII) is fused to a triabody of the antibody, or a fragment or variant thereof. In some embodiments, the TGF-β trap (e.g., TGFβRII) is fused to a triabody of the antibody, or a fragment or variant thereof, via a linker. In some embodiments, the TGF-β trap (e.g., TGFβRII) is fused to a minibody of the antibody, or a fragment or variant thereof. In some embodiments, the TGF-β trap (e.g., TGFβRII) is fused to a minibody of the antibody, or a fragment or variant thereof, via a linker. In one aspect, the TGF-β trap (e.g., TGFβRII) is fused to either the N- or C-terminus of the minibody of the antibody, or a fragment or variant thereof, via a linker.

In some embodiments, the TGF-β trap (e.g., TGFβRII) is fused to a V_{NAR} of the antibody of the first component, or a fragment or variant thereof. In some embodiments, the TGF-β trap (e.g., TGFβRII) is fused to a V_{NAR} of the antibody, or a fragment or variant thereof, via a linker. In all embodiments, as described, the TGF-β trap (e.g., TGFβRII) is fused to either the N- or C-terminus of the V_{NAR} of the antibody, or a fragment or variant thereof, via a linker.

In some embodiments, the TGF-β trap (e.g., TGFβRII) is fused to a V_{H}H of the antibody of the first component, or a fragment or variant thereof. In some embodiments, the TGF-β trap (e.g., TGFβRII) is fused to a V_{H}H of the antibody, or a fragment or variant thereof, via a linker. In all embodiments, as described, the TGF-β trap (e.g., TGFβRII) is fused to either the N- or C-terminus of the V_{H}H of the antibody, or a fragment or variant thereof, via a linker.

In some embodiments, the antibody, or fragment or variant thereof, of the first component is fused to TGFβRII or a fragment or a variant thereof (e.g., ECD of TGFβRII). In some embodiments, the antibody moiety is fused to TGFβRII or a fragment or a variant thereof (e.g., ECD of TGFβRII) via a linker. In some embodiments, the antibody moiety is fused to at least one extracellular domain of TGFβRII. In some embodiments, the antibody moiety is fused to at least one extracellular domain of TGFβRII via a linker. Non-limiting exemplary fusion proteins are illustrated in FIGS. 4A-4C.

In some embodiments, the antibody fragment is a Fab, Fab2, (Fab′)₂, Fv, (Fv)₂, scFv, scFv-Fc, Fc, diabody, triabody, or minibody of the antibody of the first component. In some embodiments, the antibody fragment is a single-domain antibody of the antibody. In some embodiments, the single-domain antibody is a V_{NAR} or V_{H}H fragment of the antibody.

In some embodiments, the variable region of heavy chain (V_{H}) of anti-PD-1 antibody, or a fragment or variant thereof, comprises one or more polypeptide sequences as shown in any one of SEQ ID NOs: 1-7, 149-164, 333-337, and 384. In some embodiments, the variable region of heavy chain (V_{H}) of anti-PD-1 antibody, or a fragment or variant thereof, comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 1-7, 149-164, 333-337, and 384.

In some embodiments, the variable region of light chain (V_{L}) of anti-PD-1 antibody, or a fragment or variant thereof, comprises one or more polypeptide sequences as shown in any one of SEQ ID NOs: 8-13, 148, 338-343, and 385. In some embodiments, the variable region of light chain (V_{L}) of anti-PD-1 antibody, or a fragment or variant thereof, comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 8-13, 148, 338-343, and 385.

In some embodiments, the fusion protein comprises a variable region of heavy chain (V_{H}) comprising a polypeptide sequence as shown in SEQ ID NO: 6 and a variable region of light chain (V_{L}) comprising a polypeptide sequence as shown in SEQ ID NO: 12. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 6 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 12.

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 16.

In some embodiments, the fusion protein comprises a sequence as shown in SEQ ID NO: 15 and a sequence as shown in SEQ ID NO: 16.

In some embodiments, the fusion protein comprises a variable region of heavy chain (V_{H}) comprising a polypeptide sequence as shown in SEQ ID NO: 333 and a variable region of light chain (V_{L}) comprising a polypeptide sequence as shown in SEQ ID NO: 338. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 333 and a variable region of light chain (V_{L}) that is at least 80%, at least 85%, at least 90%, at least 95%, at least 98%, at least 99%, or 100% identical to a sequence as shown in SEQ ID NO: 338.

In some embodiments, the fusion protein comprises a variable region of heavy chain (V_{H}) comprising a polypeptide sequence as shown in SEQ ID NO: 334 and a variable region of light chain (V_{L}) comprising a polypeptide sequence as shown in SEQ ID NO: 339. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 334 and a variable region of light chain (V_{L}) that is at least 80%, at least 85%, at least 90%, at least 95%, at least 98%, at least 99%, or 100% identical to a sequence as shown in SEQ ID NO: 339.

In some embodiments, the fusion protein comprises a variable region of heavy chain (V_{H}) comprising a polypeptide sequence as shown in SEQ ID NO: 335 and a variable region of light chain (V_{L}) comprising a polypeptide sequence as shown in SEQ ID NO: 340. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 335 and a variable region of light chain (V_{L}) that is at Icast 80%, at least 85%, at least 90%, at least 95%, at least 98%, at least 99%, or 100% identical to a sequence as shown in SEQ ID NO: 340.

In some embodiments, the fusion protein comprises a variable region of heavy chain (V_{H}) comprising a polypeptide sequence as shown in SEQ ID NO: 336 and a variable region of light chain (V_{L}) comprising a polypeptide sequence as shown in SEQ ID NO: 341. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 336 and a variable region of light chain (V_{L}) that is at least 80%, at least 85%, at least 90%, at least 95%, at least 98%, at least 99%, or 100% identical to a sequence as shown in SEQ ID NO: 341.

In some embodiments, the fusion protein comprises a variable region of heavy chain (V_{H}) comprising a polypeptide sequence as shown in SEQ ID NO: 337 and a variable region of light chain (V_{L}) comprising a polypeptide sequence as shown in SEQ ID NO: 342. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 337 and a variable region of light chain (VL) that is at least 80%, at least 85%, at least 90%, at least 95%, at least 98%, at least 99%, or 100% identical to a sequence as shown in SEQ ID NO: 342.

In some embodiments, the fusion protein comprises a variable region of heavy chain (V_{H}) comprising a polypeptide sequence as shown in SEQ ID NO: 335 and a variable region of light chain (V_{L}) comprising a polypeptide sequence as shown in SEQ ID NO: 343. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 335 and a variable region of light chain (V_{L}) that is at least 80%, at least 85%, at least 90%, at least 95%, at least 98%, at least 99%, or 100% identical to a sequence as shown in SEQ ID NO: 343.

In some embodiments, the fusion protein comprises a variable region of heavy chain (V_{H}) comprising a polypeptide sequence as shown in SEQ ID NO: 384 and a variable region of light chain (V_{L}) comprising a polypeptide sequence as shown in SEQ ID NO: 385. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 384 and a variable region of light chain (V_{L}) that is at Icast 80%, at least 85%, at least 90%, at least 95%, at least 98%, at least 99%, or 100% identical to a sequence as shown in SEQ ID NO: 385.

In some embodiments, the variable region of heavy chain (V_{H}) of a MUCI targeting antibody, or a fragment or variant thereof, comprises one or more polypeptide sequences as shown in any one of SEQ ID NOs: 444 448. In some embodiments, the variable region of heavy chain (V_{H}) of a MUCI targeting antibody, or a fragment or variant thereof, comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 444-448.

In some embodiments, the variable region of light chain (V_{L}) of a MUCI targeting antibody, or a fragment or variant thereof, comprises one or more polypeptide sequences as shown in any one of SEQ ID NOs: 449-453. In some embodiments, the variable region of light chain (V_{L}) of a MUCI targeting antibody, or a fragment or variant thereof, comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 449-453.

In some embodiments, the fusion protein comprises a variable region of heavy chain (V_{H}) comprising a polypeptide sequence as shown in SEQ ID NO: 444. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 444.

In some embodiments, the fusion protein comprises a heavy chain sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 466.

In some embodiments, the fusion protein comprises a light chain sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 467.

In some embodiments, the variable region of heavy chain (V_{H}) of a MUC16 targeting antibody, or a fragment or variant thereof, comprises one or more polypeptide sequences as shown in any one of SEQ ID NOs: 390-403. In some embodiments, the variable region of heavy chain (V_{H}) of a MUC16 targeting antibody, or a fragment or variant thereof, comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 390-403.

In some embodiments, the variable region of light chain (V_{L}) of a MUC16 targeting antibody, or a fragment or variant thereof, comprises one or more polypeptide sequences as shown in any one of SEQ ID NOs: 417-428. In some embodiments, the variable region of light chain (V_{L}) of a MUC16 targeting antibody, or a fragment or variant thereof, comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 417-428.

In some embodiments, the fusion protein comprises a variable region of heavy chain (V_{H}) comprising a polypeptide sequence as shown in SEQ ID NO: 399. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 399.

In some embodiments, the fusion protein comprises a variable region of light chain (V_{L}) comprising a polypeptide sequence as shown in SEQ ID NO: 426. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 426.

In some embodiments, the fusion protein comprises a heavy chain sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 441.

In some embodiments, the fusion protein comprises a light chain sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 443.

In certain embodiments, the fusion protein comprises a fully human monoclonal antibody that comprises a human constant region of the IgG4 isotype and a heavy chain variable region and a light chain variable region as described above.

In certain embodiments, the fusion protein comprises a heavy chain CDR comprising an amino acid sequence selected from the group consisting of: SEQ ID NOs: 310-320, 378-380, and sequences having at least 80%, at least 85%, at least 90%, at least 95%, at least 98%, at least 99%, or 100% sequence identity therewith. In certain embodiments, the fusion protein comprises a light chain CDR comprising an amino acid sequence selected from the group consisting of: SEQ ID NOs: 321-332, 381-383, and sequences having at least 80%, at least 85%, at least 90%, at least 95%, at least 98%, at least 99%, or 100% sequence identity therewith.

In certain embodiments, the fusion protein comprises at least one, two, three, four, five, or six CDRs, each comprising an amino acid sequence individually selected from: SEQ ID NOs: 310-312 and 321-323; SEQ ID NOs: 312-314, 321, 323, and 324; SEQ ID NOs: 310, 312, 314, 321, 324, and 325; SEQ ID NOs: 310, 312, 314, 321, 324, and 332; SEQ ID NOs: 315-317 and 326-328; SEQ ID NOs: 318-320 and 329-331; or SEQ ID NOs: 378-383. In certain embodiments, the fusion protein comprises a heavy chain variable region comprising the amino acid sequences of: SEQ ID NOs: 310, 311, and/or 312; SEQ ID NOs: 312, 313, and/or 314; SEQ ID NOS: 310, 312, and/or 314; SEQ ID NOs: 315, 316, and/or 317; SEQ ID NOs: 318, 319, and/or 320; or SEQ ID NOs: 378, 379, and/or 380. In certain embodiments, the fusion protein comprises a light chain variable region comprising the amino acid sequences of: SEQ ID NOs: 321, 322, and/or 323; SEQ ID NOs: 321, 323, and/or 324; SEQ ID NOs: 321, 324, and/or 325; SEQ ID NOs: 326, 327, and/or 328; SEQ ID NOs: 329, 330, and/or 331; SEQ ID NOs: 321, 324, and/or 332; or SEQ ID NOs: 381, 382, and/or 383. In certain embodiments, the fusion protein comprises: a heavy chain variable region comprising the amino acid sequences of SEQ ID NOs: 310, 311, and/or 312 and a light chain variable region comprising the amino acid sequences of SEQ ID NOs: 321, 322, and/or 323; a heavy chain variable region comprising the amino acid sequences of SEQ ID NOs: 312, 313, and/or 314 and a light chain variable region comprising the amino acid sequences of SEQ ID NOs: 321, 323, and/or 324; a heavy chain variable region comprising the amino acid sequences of SEQ ID NOs: 310, 312, and/or 314 and a light chain variable region comprising the amino acid sequences of SEQ ID NOs: 321, 324, and/or 325; a heavy chain variable region comprising the amino acid sequences of SEQ ID NOs: 315, 316, and/or 317 and a light chain variable region comprising the amino acid sequences of SEQ ID NOs: 326, 327, and/or 328; a heavy chain variable region comprising the amino acid sequences of SEQ ID NOs: 318, 319, and/or 320 and a light chain variable region comprising the amino acid sequences of SEQ ID NOs: 329, 330, and/or 331; a heavy chain variable region comprising the amino acid sequences of SEQ ID NOs: 310, 312, and/or 315 and a light chain variable region comprising the amino acid sequences of SEQ ID NOs: 321, 324, and/or 332; or a heavy chain variable region comprising the amino acid sequences of SEQ ID NOs: 378, 379, and/or 380 and a light chain variable region comprising the amino acid sequences of SEQ ID NOs: 381, 382, and/or 383. In each of the aforementioned embodiments, the CDRs may instead comprise amino acid sequences having at least 80%, at least 85%, at least 90%, at least 95%, at least 98%, or at least 99% sequence identity with the recited sequences.

In certain embodiments, the fusion protein comprises: three heavy chain CDRs, the CDRs comprising, respectively, amino acid sequences of SEQ ID NOs: 378, 379, and 380, or sequences having at least 80%, at least 85%, at least 90%, at least 95%, at least 98%, at least 99%, or 100% sequence identity therewith; and three light chain CDRs, the CDRs comprising, respectively, amino acid sequences of SEQ ID NOs: 381, 382, and 383, or sequences having at least 80%, at least 85%, at least 90%, at least 95%, at least 98%, at least 99%, or 100% sequence identity therewith.

In certain embodiments, the fusion protein comprises: three heavy chain CDRs, the CDRs comprising, respectively, amino acid sequences of SEQ ID NOs: 378, 379, and 380; and three light chain CDRs, the CDRs comprising, respectively, amino acid sequences of SEQ ID NOS: 381, 382, and 383.

In certain embodiments, the fusion protein comprises a heavy chain comprising an amino acid sequence of SEQ ID NO: 386, or a sequence having at least 80%, at least 85%, at least 90%, at least 95%, at least 98%, at least 99%, or 100% sequence identity therewith, and a light chain comprising an amino acid sequence of SEQ ID NO: 387, or a sequence having at least 80%, at least 85%, at least 90%, at least 95%, at least 98%, at least 99%, or 100% sequence identity therewith. In certain embodiments, the fusion protein comprises a heavy chain comprising and amino acid sequence of SEQ ID NO: 386 and a light chain comprising an amino acid sequence of SEQ ID NO: 387.

In some embodiments of the present invention, the fusion protein comprises an anti-PD-1 moiety selected or derived from a full-length, fragment, or variant of one or more antibody from Table 1.

In some embodiments, the fusion protein comprises an anti-PD-1 moiety that is selected or derived from a full-length, fragment, or variant of pembrolizumab (Keytruda®), nivolumab (Opdivo®), zimberelimab (WBP3055), or cetrelimab (JNJ-63723283).

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 143.

In some embodiments, the fusion protein comprises a sequence as shown in SEQ ID NO: 15 and a sequence as shown in SEQ ID NO: 143.

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 294.

In embodiments, the fusion protein comprises a sequence as shown in SEQ ID NO: 15 and a sequence as shown in SEQ ID NO: 294.

In some embodiments, the fusion protein comprises a variable region of heavy chain (V_{H}) comprising a polypeptide sequence as shown in SEQ ID NO: 7 and a variable region of light chain (V_{L}) comprising a polypeptide sequence as shown in SEQ ID NO: 13. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 7 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 13.

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 296 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 145.

In some embodiments, the fusion protein comprises a sequence as shown in SEQ ID NO: 296 and a sequence as shown in SEQ ID NO: 145.

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 296 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 144.

In some embodiments, the fusion protein comprises a sequence as shown in SEQ ID NO: 296 and a sequence as shown in SEQ ID NO: 144.

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 296 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 295.

In some embodiments, the fusion protein comprises a sequence as shown in SEQ ID NO: 296 and a sequence as shown in SEQ ID NO: 295.

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 12 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 16.

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 12 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 143.

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 13 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 145. In some embodiments, the fusion protein comprises a sequence as shown in SEQ ID NO: 145.

In some embodiments, the fusion protein comprises a linker and a variable region of heavy chain (V_{H}) comprising a polypeptide sequence as shown in SEQ ID NO: 16 and a variable region of light chain (V_{L}) comprising a polypeptide sequence as shown in SEQ ID NO: 15. In some embodiments, the fusion protein comprises a linker and a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 16 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15.

In some embodiments, the fusion protein comprises a variable region of heavy chain (V_{H}) comprising a polypeptide sequence as shown in SEQ ID NO: 1. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 1.

In some embodiments, the fusion protein comprises a variable region of heavy chain (V_{H}) comprising a polypeptide sequence as shown in SEQ ID NO: 2. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 2.

In some embodiments, the fusion protein comprises a variable region of heavy chain (V_{H}) comprising a polypeptide sequence as shown in SEQ ID NO: 3. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 3.

In some embodiments, the fusion protein comprises a variable region of heavy chain (V_{H}) comprising a polypeptide sequence as shown in SEQ ID NO: 4. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 4.

In some embodiments, the fusion protein comprises a variable region of heavy chain (V_{H}) comprising a polypeptide sequence as shown in SEQ ID NO: 5. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 5.

In some embodiments, the fusion protein comprises a variable region of heavy chain (V_{H}) comprising a polypeptide sequence as shown in SEQ ID NO: 6. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 6.

In some embodiments, the fusion protein comprises a variable region of heavy chain (V_{H}) comprising a polypeptide sequence as shown in SEQ ID NO: 7. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 7.

In some embodiments, the fusion protein comprises a variable region of heavy chain (V_{H}) comprising a polypeptide sequence as shown in SEQ ID NO: 5 and a variable region of light chain (V_{L}) comprising a polypeptide sequence as shown in SEQ ID NO: 8. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 5 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 8.

In some embodiments, the fusion protein comprises a variable region of heavy chain (V_{H}) comprising a polypeptide sequence as shown in SEQ ID NO: 149 and a variable region of light chain (V_{L}) comprising a polypeptide sequence as shown in SEQ ID NO: 8. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 149 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 8.

In some embodiments, the fusion protein comprises a variable region of heavy chain (V_{H}) comprising a polypeptide sequence as shown in SEQ ID NO: 157 and a variable region of light chain (V_{L}) comprising a polypeptide sequence as shown in SEQ ID NO: 8. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 157 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 8.

In some embodiments, the fusion protein comprises a variable region of heavy chain (V_{H}) comprising a polypeptide sequence as shown in SEQ ID NO: 158 and a variable region of light chain (V_{L}) comprising a polypeptide sequence as shown in SEQ ID NO: 8. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 158 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 8.

In some embodiments, the fusion protein comprises a variable region of heavy chain (V_{H}) comprising a polypeptide sequence as shown in SEQ ID NO: 5. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 5.

In some embodiments, the fusion protein comprises a variable region of heavy chain (V_{H}) comprising a polypeptide sequence as shown in SEQ ID NO: 149. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 149.

In some embodiments, the fusion protein comprises a variable region of heavy chain (V_{H}) comprising a polypeptide sequence as shown in SEQ ID NO: 157. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 157.

In some embodiments, the fusion protein comprises a variable region of heavy chain (V_{H}) comprising a polypeptide sequence as shown in SEQ ID NO: 158. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 158.

In some embodiments, the fusion protein comprises a variable region of heavy chain (V_{H}) comprising a polypeptide sequence as shown in SEQ ID NO: 158. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 158.

In some embodiments, the fusion protein comprises a variable region of light chain (V_{L}) comprising a polypeptide sequence as shown in SEQ ID NO: 8. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 8.

In some embodiments, the fusion protein comprises a variable region of heavy chain (V_{H}) comprising a polypeptide sequence as shown in SEQ ID NO: 149. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 149.

In some embodiments, the fusion protein comprises a variable region of heavy chain (V_{H}) comprising a polypeptide sequence as shown in SEQ ID NO: 150. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 150.

In some embodiments, the fusion protein comprises a variable region of heavy chain (V_{H}) comprising a polypeptide sequence as shown in SEQ ID NO: 151. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 151.

In some embodiments, the fusion protein comprises a variable region of heavy chain (V_{H}) comprising a polypeptide sequence as shown in SEQ ID NO: 152. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 152.

In some embodiments, the fusion protein comprises a variable region of heavy chain (V_{H}) comprising a polypeptide sequence as shown in SEQ ID NO: 153. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 153.

In some embodiments, the fusion protein comprises a variable region of heavy chain (V_{H}) comprising a polypeptide sequence as shown in SEQ ID NO: 154. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 154.

In some embodiments, the fusion protein comprises a variable region of heavy chain (V_{H}) comprising a polypeptide sequence as shown in SEQ ID NO: 155. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 155.

In some embodiments, the fusion protein comprises a variable region of heavy chain (V_{H}) comprising a polypeptide sequence as shown in SEQ ID NO: 156. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 156.

In some embodiments, the fusion protein comprises a variable region of heavy chain (V_{H}) comprising a polypeptide sequence as shown in SEQ ID NO: 157. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 157.

In some embodiments, the fusion protein comprises a variable region of heavy chain (V_{H}) comprising a polypeptide sequence as shown in SEQ ID NO: 158. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 158.

In some embodiments, the fusion protein comprises a variable region of heavy chain (V_{H}) comprising a polypeptide sequence as shown in SEQ ID NO: 159. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 159.

In some embodiments, the fusion protein comprises a variable region of heavy chain (V_{H}) comprising a polypeptide sequence as shown in SEQ ID NO: 160. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 160.

In some embodiments, the fusion protein comprises a variable region of heavy chain (V_{H}) comprising a polypeptide sequence as shown in SEQ ID NO: 161. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 161.

In some embodiments, the fusion protein comprises a variable region of heavy chain (V_{H}) comprising a polypeptide sequence as shown in SEQ ID NO: 162. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 162.

In some embodiments, the fusion protein comprises a variable region of heavy chain (V_{H}) comprising a polypeptide sequence as shown in SEQ ID NO: 163. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 163.

In some embodiments, the fusion protein comprises a variable region of heavy chain (V_{H}) comprising a polypeptide sequence as shown in SEQ ID NO: 164. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 164.

In some embodiments, the fusion protein comprises a variable region of light chain (V_{L}) comprising a polypeptide sequence as shown in SEQ ID NO: 8. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 8.

In some embodiments, the fusion protein comprises a variable region of light chain (V_{L}) comprising a polypeptide sequence as shown in SEQ ID NO: 9. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 9.

In some embodiments, the fusion protein comprises a variable region of light chain (V_{L}) comprising a polypeptide sequence as shown in SEQ ID NO: 10. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 10.

In some embodiments, the fusion protein comprises a variable region of light chain (V_{L}) comprising a polypeptide sequence as shown in SEQ ID NO: 11. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 11.

In some embodiments, the fusion protein comprises a variable region of light chain (V_{L}) comprising a polypeptide sequence as shown in SEQ ID NO: 12. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 12.

In some embodiments, the fusion protein comprises a variable region of light chain (V_{L}) comprising a polypeptide sequence as shown in SEQ ID NO: 13. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 13.

In some embodiments, the fusion protein comprises a variable region of light chain (V_{L}) comprising a polypeptide sequence as shown in SEQ ID NO: 148. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 148.

In some embodiments, the fusion protein comprises a polypeptide sequence as shown in SEQ ID NO: 15. In some embodiments, the fusion protein comprises a polypeptide sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15.

In some embodiments, the fusion protein comprises a polypeptide sequence as shown in SEQ ID NO: 16. In some embodiments, the fusion protein comprises a polypeptide sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 16.

In some embodiments, the fusion protein comprises a polypeptide sequence as shown in SEQ ID NO: 143. In some embodiments, the fusion protein comprises a polypeptide sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 143.

In some embodiments, the fusion protein comprises a polypeptide sequence as shown in SEQ ID NO: 144. In some embodiments, the fusion protein comprises a polypeptide sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 144.

In some embodiments, the fusion protein comprises a polypeptide sequence as shown in SEQ ID NO: 145. In some embodiments, the fusion protein comprises a polypeptide sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 145.

### Anti-TGF β Antibodies-Fusion Proteins, or Fragments or Variants Thereof

In other embodiments, the cytokine trap is an antibody, antibody fragment or an antibody variant against TGF-β. In such embodiments, such antibodies can include pan-neutralizing anti-TGFB antibody, or an anti-receptor antibody that blocks the receptor from binding to TGFβ1, 2 and/or 3. In certain embodiments, the antibody fragment or variant is a Fab, Fab2, (Fab′)₂, Fv, (Fv)₂, scFv, scFv-Fc, Fc, diabody, triabody, or minibody of the TGFβ antibody. In some embodiments, the anti-TGF-β antibody or fragment or variant thereof binds to TGF-β1, TGF-β2, and TGF-β3. In certain embodiments, the anti-TGF-β antibody or fragment or variant thereof binds to TGF-β1. In certain embodiments, the anti-TGF-β antibody or fragment or variant thereof binds to TGF-β3. In certain embodiments, the anti-TGF-β antibody or fragment or variant thereof binds to TGF-β1 and TGF-β2. In certain embodiments, the anti-TGF-β antibody or fragment or variant thereof binds to TGF-β1 and TGF-β3. Examples of V_{H} sequence of a TGF-β antibody or fragment or variant thereof include but are not limited to SEQ ID Nos. 166, 168, 169, 171, 173, 175, and 177. Examples of V_{L} sequence of a TGF-β antibody or fragment or variant thereof include but are not limited to SEQ ID NOS: 165, 167, 170, 172, 174, 176, and 178. In some embodiments, the anti-TGF-β antibody comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence selected from any one of SEQ ID NOs: 166, 168, 169, 171, 173, 175, or 177. In some embodiments, the anti-TGF-β antibody comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence selected from any one of SEQ ID NOs: 165, 167, 170, 172, 174, 176, or 178.

### TGF-β Antagonistic Peptides-Fusion Proteins, or Fragments or Variants Thereof

In certain embodiments, the cytokine trap can include TGF-β antagonistic peptides or TGF-β inhibitory peptides. Such peptides can be generated de novo using phage display. In some embodiments, such peptides can be derived from segments of TGF-β isoform(s) or TGF-β receptor(s), Examples of TGF-β antagonistic peptides can include but are not limited to SEQ ID NOs: 468-507 and 263-267. Examples of TGF-β inhibitory peptides can include but are not limited to SEQ ID NOs: 468-507 and 263-267. In some embodiments, the peptides can be fused to V_{H} and/or V_{L} of the antibody of the first component, or a fragment or variant thereof, via a linker. In another embodiment, one peptide can be fused to the antibody of the first component, or a fragment or variant thereof. In a further embodiment, more than one peptide can be fused to the antibody of the first component, or a fragment or variant thereof. When more than one peptide is utilized, the peptide can be fused with or without a linker to form concatemers. When more than one peptide is utilized, the same peptide can be used to form concatemers. Alternatively, a combination of two or more different peptides can be used to form concatemers.

### Adenosine

Adenosine is a key immune modulator in the tumor microenvironment. Extracellular adenosine suppresses inflammatory response upon binding to the A2A adenosine receptor (A2AR) which is predominantly expressed subtype in most immune cells. Several tumors express high levels of CD39 and CD73, the ectonucleotidases responsible for converting ATP and ADP to AMP, and AMP to adenosine, respectively. Accordingly, adenosine promotes suppressive activity of regulatory T cells by inducing the expression of Foxp3, CD39 and CD73. Additionally, hypoxia induces the accumulation of extracellular adenosine in the tumor microenvironment through the induction of CD39 and CD73. In addition, high levels of extracellular adenosine in the tumor microenvironment is maintained by the hypoxia-inducible factor (HIF)-dependent inhibition of the nucleotide transporter ENT-1, and the inhibition of adenosine kinase preventing the relocation of adenosine in the intracellular space and preventing the formation of AMP, respectively. Accordingly, targeting the reduction of extracellular adenosine in the tumor microenvironment is understood to enhance immune cell function and promote tumor cell killing.

In embodiments provided herein are fusion proteins that comprise an adenosine deaminase (e.g., ADA2) to target reduction of extracellular adenosine in the tumor microenvironment.

### Adenosine Deaminase

Adenosine deaminase (also known as adenosine aminohydrolase, or ADA) ADA irreversibly deaminates adenosine, converting it to the related nucleoside inosine by the substitution of the amino group for a keto group. Inosine can then be deribosylated (removed from ribose) by another enzyme called purine nucleoside phosphorylase (PNP), converting it to hypoxanthine. ADA is needed for the breakdown of adenosine from food and for the turnover of nucleic acids in tissues. Its primary function in humans is the development and maintenance of the immune system. However, ADA association has also been observed with epithelial cell differentiation, neurotransmission, and gestation maintenance. There are 2 isoforms of ADA: ADA1 and ADA2.

ADAI is found in most body cells, particularly lymphocytes and macrophages, where it is present not only in the cytosol and nucleus but also as the ecto-form on the cell membrane attached to dipeptidyl peptidase-4 (aka, CD26). ADAI is involved mostly in intracellular activity, and exists both in small form (monomer) and large form (dimer). The interconversion of small to large forms is regulated by a ‘conversion factor’ in the lung.

ADA2 was first identified in human spleen. It was subsequently found in other tissues including the macrophage where it co-exists with ADA1. The two isoforms regulate the ratio of adenosine to deoxyadenosine. ADA2 is found predominantly in the human plasma and serum, and exists mainly as a homodimer. ADA2 is the predominant form present in human blood plasma and is increased in many diseases, particularly those associated with the immune system: for example rheumatoid arthritis, psoriasis, and sarcoidosis. The plasma ADA2 isoform is also increased in most cancers. ADA2 is not ubiquitous but co-exists with ADAI in monocytes-macrophages.

### ADA2 Fusion Proteins, or Fragments or Variants Thereof

Provided herein is a fusion protein or a fragment or a variant thereof comprising: (1) an immune checkpoint inhibitor, such as a PD-1 inhibitor or antibody, or an antibody that binds a tumor antigen expressed on the surface of a tumor cell; and (2) an adenosine deaminase (e.g., ADA2) that can neutralize adenosine. The fusion protein or a fragment or a variant thereof provided herein (e.g., PD-1 inhibitor or antibody fused to an adenosine deaminase (e.g., ADA2) can elicit a synergistic anti-tumor effect due to the simultaneous blockade of the interaction between e.g., PD-L1, MUCI, and MUC16 on tumor cells on tumor cells and PD-1, MUCI, and MUC16 on tumor cells on immune cells, and the neutralization of e.g., adenosine in the tumor microenvironment. Without being bound by theory, this effect is obtained from simultaneous blocking the two major immune escape mechanisms and the targeted depletion of adenosine in the tumor microenvironment by a single molecular entity. This depletion can be achieved by one or more of the following: (1) anti-PD-1, anti-MUCI, or anti-MUC16 targeting of tumor cells; (2) binding of the adenosine in the tumor microenvironment by an adenosine deaminase (e.g., ADA2); and (3) destruction of the bound adenosine through the PD-L1 receptor mediated endocytosis.

In some embodiments, the adenosine deaminase (e.g., ADA2) is a part of the fusion protein or a fragment or a variant thereof. In some embodiments the fusion protein or a fragment or a variant thereof comprises the adenosine deaminase (e.g., ADA2) fused to the antibody of the first component optionally via a cleavable or non-cleavable linker. In some embodiments, an adenosine deaminase is an adenosine deaminase 2 (ADA2). In some embodiments, the fusion protein described herein comprises an adenosine deaminase (e.g., ADA2) described herein or a functional variant or derivative thereof. Examples of ADA2 and variants are described in WO 2016061286, herein incorporated by reference in its entirety. In some embodiments, the TGF-β cytokine trap comprises any one of ADA2 mutant 1, ADA2 mutant 2, ADA2 mutant 3, ADA2 mutant 4, ADA2 mutant 5, ADA2 mutant 6, or ADA2 mutant 7. In some embodiments, the fusion protein or a fragment or a variant thereof provided herein comprises the antibody fused to the ADA protein, or fragment thereof, via a linker.

In some embodiments, the fusion protein or a fragment or a variant thereof described herein comprises a polypeptide sequence having at least 50%, at least 55%, at least 60%, at least 65%, at least 70%, at least 75%, at least 80%, at least 85%, at least 90%, at least 95%, at least 99%, at least 99.5% or 100% identity with the polypeptide sequence of SEQ ID NO: 284. In some embodiments, the fusion protein or a fragment or a variant thereof described herein comprises a polypeptide sequence having at least 50%, at least 55%, at least 60%, at least 65%, at least 70%, at least 75%, at least 80%, at least 85%, at least 90%, at least 95%, at least 99%, at least 99.5% or 100% identity with the polypeptide sequence of SEQ ID NO: 273. In some embodiments, the fusion protein or a fragment or a variant thereof described herein comprises a polypeptide sequence having at least 50%, at least 55%, at least 60%, at least 65%, at least 70%, at least 75%, at least 80%, at least 85%, at least 90%, at least 95%, at least 99%, at least 99.5% or 100% identity with the polypeptide sequence of SEQ ID NO: 274. In some embodiments, the fusion protein or a fragment or a variant thereof described herein comprises a polypeptide sequence having at least 50%, at least 55%, at least 60%, at least 65%, at least 70%, at least 75%, at least 80%, at least 85%, at least 90%, at least 95%, at least 99%, at least 99.5% or 100% identity with the polypeptide sequence of SEQ ID NO: 275. In some embodiments, the fusion protein or a fragment or a variant thereof described herein comprises a polypeptide sequence having at least 50%, at least 55%, at least 60%, at least 65%, at least 70%, at least 75%, at least 80%, at least 85%, at least 90%, at least 95%, at least 99%, at least 99.5% or 100% identity with the polypeptide sequence of SEQ ID NO: 276. In some embodiments, the fusion protein or a fragment or a variant thereof described herein comprises a polypeptide sequence having at least 50%, at least 55%, at least 60%, at least 65%, at least 70%, at least 75%, at least 80%, at least 85%, at least 90%, at least 95%, at least 99%, at least 99.5% or 100% identity with the polypeptide sequence of SEQ ID NO: 277. In some embodiments, the fusion protein or a fragment or a variant thereof described herein comprises a polypeptide sequence having at least 50%, at least 55%, at least 60%, at least 65%, at least 70%, at least 75%, at least 80%, at least 85%, at least 90%, at least 95%, at least 99%, at least 99.5% or 100% identity with the polypeptide sequence of SEQ ID NO: 278. In some embodiments, the fusion protein or a fragment or a variant thereof described herein comprises a polypeptide sequence having at least 50%, at least 55%, at least 60%, at least 65%, at least 70%, at least 75%, at least 80%, at least 85%, at least 90%, at least 95%, at least 99%, at least 99.5% or 100% identity with the polypeptide sequence of SEQ ID NO: 279.

In some embodiments, the fusion protein or a fragment or a variant thereof described herein comprises an adenosine deaminase (e.g., ADA2) as described above and an antibody, a fragment or a variant of the antibody that targets immune checkpoint genes. In some embodiments, an antibody or a fragment or a variant of the antibody that target immune checkpoints, such as cytotoxic T lymphocyte associated protein-4 (CTLA-4) and programmed cell death-1 (PD-1), or an antibody that binds to a tumor antigen expressed on the surface of a tumor cell can be fused to an adenosine deaminase (e.g., ADA2) molecule via a linker.

In some embodiments, an antibody, or fragment or variant thereof, of the first component is fused to an adenosine deaminase (e.g., ADA2) or a fragment thereof. In some embodiments, the antibody moiety is fused to an adenosine deaminase (e.g., ADA2) via a linker. In some embodiments, the antibody moiety is fused to at least one domain of ADA2. In some embodiments, the antibody moiety is fused to at least one domain of ADA2 via a linker.

In some embodiments, the antibody of the first component, or fragment or variant thereof, is a Fab, Fab2, (Fab′)₂, Fv, (Fv)₂, scFv, scFv-Fc, Fc, diabody, triabody, or minibody of the antibody. In some embodiments, the antibody fragment is a single-domain antibody of the antibody. In some embodiments, the single-domain antibody is a V_{NAR} or V_{H}H fragment of the antibody.

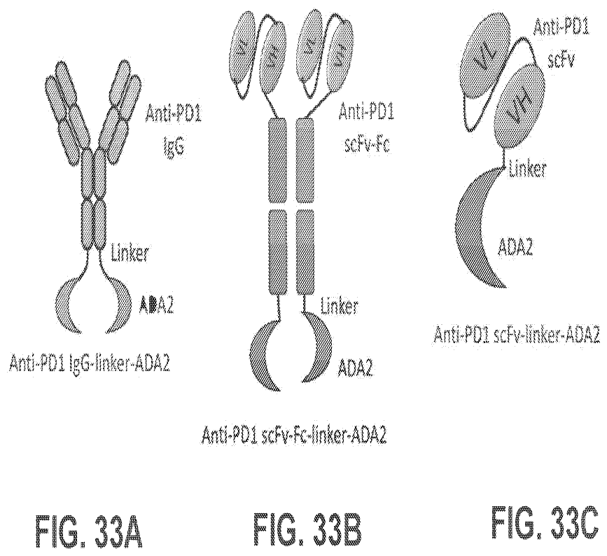

Non-limiting exemplary fusion proteins comprising anti-PD-1 are illustrated in FIGS. 33A-33C. In some embodiments, the fusion protein comprising an antibody or a fragment or a variant thereof fused to an adenosine deaminase (e.g., ADA2) can elicit a synergistic anti-tumor effect due to the simultaneous blockade of the interaction between PD-L1 on tumor cells or PD-1 on immune cells or anti-MUCI or anti-MUC16 targeting of tumor cells, and targeting of the reduction of extracellular adenosine in the tumor microenvironment.

In some embodiments, the adenosine deaminase (e.g., ADA2) is fused to a variable region of heavy chain (V_{H}) of the antibody of the first component, or a fragment or variant thereof. In other embodiments, the adenosine deaminase is fused to IgG of the antibody (for example, FIG. 4a). In certain aspects, the IgG is IgG1, IgG2, IgG3, or lgG4. In an embodiment, the IgG is IgG4. In another embodiment, the IgG4 is SEQ ID NO 146 (wild type), SEQ ID NO: 291, SEQ ID NO: 292 or SEQ ID NO: 147 (S108P). In some embodiments, the adenosine deaminase (e.g., ADA2) is fused to a variable region of heavy chain (V_{H}) of the antibody, or a fragment or variant thereof, via a linker. In some embodiments, the adenosine deaminase (e.g., ADA2) is fused to a constant region of the V_{H} of the antibody, or a fragment or variant thereof, via a linker. In some embodiments, the adenosine deaminase (e.g., ADA2) is fused a variable region of light chain (V_{L}) of the antibody, or a fragment or variant thereof. In some embodiments, the adenosine deaminase (e.g., ADA2) is fused a variable region of light chain (V_{L}) of the antibody, or a fragment or variant thereof, via a linker. In some embodiments, the adenosine deaminase (e.g., ADA2) is fused to a constant region of the V_{L}, of the antibody, or a fragment or variant thereof, via a linker. In one aspect, the adenosine deaminase is fused to either the N- or C-terminus of the V_{H} or V_{L} of the antibody, or a fragment or variant thereof, via a linker.

In some embodiments, the adenosine deaminase (e.g., ADA2) is fused to a Fab of the antibody of the first component, or a fragment or variant thereof. In some embodiments, the adenosine deaminase (e.g., ADA2) is fused to a Fab of the antibody, or a fragment or variant thereof, via a linker. In some embodiments, the adenosine deaminase (e.g., ADA2) is fused to a Fab2 of the antibody, or a fragment or variant thereof. In some embodiments, the adenosine deaminase (e.g., ADA2) is fused to a Fab2 of the antibody, or a fragment or variant thereof, via a linker. In some embodiments, the adenosine deaminase (e.g., ADA2) is fused to a (Fab′)₂ of the antibody, or a fragment or variant thereof. In some embodiments, the adenosine deaminase (e.g., ADA2) is fused to a (Fab′)₂ of the antibody, or a fragment or variant thereof, via a linker. In one aspect, the adenosine deaminase is fused to either the N- or C-terminus of the Fab or Fab2 of the antibody, or a fragment or variant thereof, via a linker.

In some embodiments, the adenosine deaminase (e.g., ADA2) is fused to a Fv of the antibody of the first component, or a fragment or variant thereof. In some embodiments, the adenosine deaminase (e.g., ADA2) is fused to a Fv of the antibody, or a fragment or variant thereof, via a linker. In some embodiments, the adenosine deaminase (e.g., ADA2) is fused to a (Fv)₂ of the antibody, or a fragment or variant thereof. In some embodiments, the adenosine deaminase (e.g., ADA2) is fused to a (Fv)₂ of the antibody, or a fragment or variant thereof, via a linker. In one aspect, the adenosine deaminase is fused to either the N- or C-terminus of the Fv or (Fv)₂ of the antibody, or a fragment or variant thereof, via a linker.

In some embodiments, the adenosine deaminase (e.g., ADA2) is fused to a scFv of the antibody of the first component, or a fragment or variant thereof. In some embodiments, the adenosine deaminase (e.g., ADA2) is fused to a scFv of the antibody, or a fragment or variant thereof, via a linker. In some embodiments, the adenosine deaminase (e.g., ADA2) is fused to a scFv-Fc of the antibody, or a fragment or variant thereof. In some embodiments, the adenosine deaminase (e.g., ADA2) is fused to a scFv-Fc of the antibody, or a fragment or variant thereof, via a linker. In one aspect, the adenosine deaminase is fused to either the N- or C-terminus of the scFv or scFv-Fc of the antibody, or a fragment or variant thereof, via a linker.

In some embodiments, the adenosine deaminase (e.g., ADA2) is fused to a Fc of the antibody of the first component, or a fragment or variant thereof. In some embodiments, the adenosine deaminase (e.g., ADA2) is fused to a Fc of the antibody, or a fragment or variant thereof, via a linker. In some embodiments, the adenosine deaminase (e.g., ADA2) is linked to a C-terminus Fc of the antibody, or a fragment or variant thereof. In some embodiments, the adenosine deaminase (e.g., ADA2) is linked to a C-terminus Fc of the antibody, or a fragment or variant thereof, via a linker. In some embodiments, the adenosine deaminase (e.g., ADA2) is linked to a N-terminus Fc of the antibody, or a fragment or variant thereof. In some embodiments, the adenosine deaminase (e.g., ADA2) is linked to an N-terminus Fc of the antibody, or a fragment or variant thereof, via a linker.

In some embodiments, the adenosine deaminase (e.g., ADA2) is fused to a diabody of the antibody of the first component, or a fragment or variant thereof. In some embodiments, the adenosine deaminase (e.g., ADA2) is fused to a diabody of the antibody, or a fragment or variant thereof, via a linker. In some embodiments, the adenosine deaminase (e.g., ADA2) is fused to a triabody of the antibody, or a fragment or variant thereof. In some embodiments, the adenosine deaminase (e.g., ADA2) is fused to a triabody of the antibody, or a fragment or variant thereof, via a linker. In some embodiments, the adenosine deaminase (e.g., ADA2) is fused to a minibody of the antibody, or a fragment or variant thereof. In some embodiments, the adenosine deaminase (e.g., ADA2) is fused to a minibody of the antibody, or a fragment or variant thereof, via a linker.

In some embodiments, the adenosine deaminase (e.g., ADA2) is fused to a V_{NAR} of the antibody of the first component, or a fragment or variant thereof. In some embodiments, the adenosine deaminase (e.g., ADA2) is fused to a V_{NAR} of the antibody, or a fragment or variant thereof, via a linker. In one aspect, the adenosine deaminase is fused to either the N- or C-terminus of the V_{NAR} of the antibody, or a fragment or variant thereof, via a linker.

In some embodiments, the adenosine deaminase (e.g., ADA2) is fused to a V_{H}H of the antibody of the first component, or a fragment or variant thereof. In some embodiments, the adenosine deaminase (e.g., ADA2) is fused to a V_{H}H of the antibody, or a fragment or variant thereof, via a linker. In one aspect, the adenosine deaminase is fused to either the N- or C-terminus of the V_{H}H of the antibody, or a fragment or variant thereof, via a linker.

In some embodiments, the variable region of heavy chain (V_{H}) of anti-PD-1 antibody, or a fragment or variant thereof, comprises one or more polypeptide sequences as shown in any one of SEQ ID NOs: 1-7, 149-164, 333-337, and 384. In some embodiments, the variable region of heavy chain (V_{H}) of anti-PD-1 antibody, or a fragment or variant thereof, comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 1-7, 149-164, 333-337, and 384.

In some embodiments, the variable region of light chain (V_{L}) of anti-PD-1 antibody, or a fragment or variant thereof, comprises one or more polypeptide sequences as shown in any one of SEQ ID NOs: 8-13, 148, 338-343, and 385. In some embodiments, the variable region of light chain (V_{L}) of anti-PD-1 antibody, or a fragment or variant thereof, comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 8-13, 148, 338-343, and 385.

In some embodiments, the fusion protein comprises a variable region of heavy chain (V_{H}) comprising a polypeptide sequence as shown in SEQ ID NO: 6 and a variable region of light chain (V_{L}) comprising a polypeptide sequence as shown in SEQ ID NO: 12. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 6 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 12.

In some embodiments, the fusion protein comprises a variable region of heavy chain (V_{H}) comprising a polypeptide sequence as shown in SEQ ID NO: 333 and a variable region of light chain (V_{L}) comprising a polypeptide sequence as shown in SEQ ID NO: 338. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 333 and a variable region of light chain (V_{L}) that is at least 80%, at least 85%, at least 90%, at least 95%, at least 98%, at least 99%, or 100% identical to a sequence as shown in SEQ ID NO: 338.

In some embodiments, the fusion protein comprises a variable region of heavy chain (V_{H}) comprising a polypeptide sequence as shown in SEQ ID NO: 334 and a variable region of light chain (V_{L}) comprising a polypeptide sequence as shown in SEQ ID NO: 339. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 334 and a variable region of light chain (V_{L}) that is at to a sequence as shown in SEQ ID NO: 339.

In some embodiments, the fusion protein comprises a variable region of heavy chain (V_{H}) comprising a polypeptide sequence as shown in SEQ ID NO: 335 and a variable region of light chain (V_{L}) comprising a polypeptide sequence as shown in SEQ ID NO: 340. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 335 and a variable region of light chain (V_{L}) that is at Icast 80%, at least 85%, at least 90%, at least 95%, at least 98%, at least 99%, or 100% identical to a sequence as shown in SEQ ID NO: 340.

In some embodiments, the fusion protein comprises a variable region of heavy chain (V_{H}) comprising a polypeptide sequence as shown in SEQ ID NO: 336 and a variable region of light chain (V_{L}) comprising a polypeptide sequence as shown in SEQ ID NO: 341. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 336 and a variable region of light chain (V_{L}) that is at least 80%, at least 85%, at least 90%, at least 95%, at least 98%, at least 99%, or 100% identical to a sequence as shown in SEQ ID NO: 341.

In some embodiments, the fusion protein comprises a variable region of heavy chain (V_{H}) comprising a polypeptide sequence as shown in SEQ ID NO: 337 and a variable region of light chain (V_{L}) comprising a polypeptide sequence as shown in SEQ ID NO: 342. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 337 and a variable region of light chain (V_{L}) that is at least 80%, at least 85%, at least 90%, at least 95%, at least 98%, at least 99%, or 100% identical to a sequence as shown in SEQ ID NO: 342.

In some embodiments, the fusion protein comprises a variable region of heavy chain (V_{H}) comprising a polypeptide sequence as shown in SEQ ID NO: 335 and a variable region of light chain (V_{L}) comprising a polypeptide sequence as shown in SEQ ID NO: 343. In some embodiments, the fusion protein comprises a variable region of heavy chain (V_{H}) that is at least 80%, at least 85%, at least 90%, a at least 95%, at least 98%, at least 99%, or 100% identical to a sequence as shown in SEQ ID NO: 335 and a variable region of light chain (V_{L}) that is at to a sequence as shown in SEQ ID NO: 343.

In some embodiments, the fusion protein comprises a variable region of heavy chain (V_{H}) comprising a polypeptide sequence as shown in SEQ ID NO: 384 and a variable region of light chain (V_{L}) comprising a polypeptide sequence as shown in SEQ ID NO: 385. In some embodiments, the fusion protein comprises a variable region of heavy chain (V_{H}) that is at least 80%, at least 85%, at least 90%, at least 95%, at least 98%, at least 99%, or 100% identical to a sequence as shown in SEQ ID NO: 384 and a variable region of light chain (V_{L}) that is at Icast 80%, at least 85%, at least 90%, at least 95%, at least 98%, at least 99%, or 100% identical to a sequence as shown in SEQ ID NO: 385.

In some embodiments, the variable region of heavy chain (V_{H}) of a MUCI targeting antibody, or a fragment or variant thereof, comprises one or more polypeptide sequences as shown in any one of SEQ ID NOs: 444-448. In some embodiments, the variable region of heavy chain (V_{H}) of a MUCI targeting antibody, or a fragment or variant thereof, comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 444-448.

In some embodiments, the variable region of light chain (V_{L}) of a MUCI targeting antibody, or a fragment or variant thereof, comprises one or more polypeptide sequences as shown in any one of SEQ ID NOs: 449-453. In some embodiments, the variable region of light chain (V_{L}) of a MUCI targeting antibody, or a fragment or variant thereof, comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOS: 449-453.

In some embodiments, the fusion protein comprises a variable region of heavy chain (V_{H}) comprising a polypeptide sequence as shown in SEQ ID NO: 444. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 444.

In some embodiments, the fusion protein comprises a heavy chain sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 466.

In some embodiments, the fusion protein comprises a light chain sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 467.

In some embodiments, the variable region of heavy chain (V_{H}) of a MUC16 targeting antibody, or a fragment or variant thereof, comprises one or more polypeptide sequences as shown in any one of SEQ ID NOs: 390-403. In some embodiments, the variable region of heavy chain (V_{H}) of a MUC16 targeting antibody, or a fragment or variant thereof, comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 390-403.

In some embodiments, the variable region of light chain (V_{L}) of a MUC16 targeting antibody, or a fragment or variant thereof, comprises one or more polypeptide sequences as shown in any one of SEQ ID NOs: 417-428. In some embodiments, the variable region of light chain (V_{L}) of a MUC16 targeting antibody, or a fragment or variant thereof, comprises one or more polypeptide sequences having at least 50%, at least 55%, at least 60%, at least 65%, at least 70%, at least 75%, at least 80%, at least 85%, at least 90%, at least 95%, at least 99%, at least 99.5% or 100% identity with any one of polypeptide sequences as shown in SEQ ID NOs: 417-428.

In some embodiments, the fusion protein comprises a variable region of heavy chain (V_{H}) comprising a polypeptide sequence as shown in SEQ ID NO: 399. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 399.

In some embodiments, the fusion protein comprises a variable region of light chain (V_{L}) comprising a polypeptide sequence as shown in SEQ ID NO: 426. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 426.

In some embodiments, the fusion protein comprises a heavy chain sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 441.

In some embodiments, the fusion protein comprises a light chain sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 443.

In certain embodiments, the fusion protein comprises a fully human monoclonal antibody that comprises a human constant region of the IgG4 isotype and a heavy chain variable region and a light chain variable region as described above.

In certain embodiments, the fusion protein comprises a heavy chain CDR comprising an amino acid sequence selected from the group consisting of: SEQ ID NOs: 310-320, 378-380, and sequences having at least 80%, at least 85%, at least 90%, at least 95%, at least 98%, at least 99%, or 100% sequence identity therewith. In certain embodiments, the fusion protein comprises a light chain CDR comprising an amino acid sequence selected from the group consisting of: SEQ ID NOs: 321-332, 381-383, and sequences having at least 80%, at least 85%, at least 90%, at least 95%, at least 98%, at least 99%, or 100% sequence identity therewith.

In certain embodiments, the fusion protein comprises at least one, two, three, four, five, or six CDRs, each comprising an amino acid sequence individually selected from: SEQ ID NOs: 310-312 and 321-323: SEQ ID NOs: 312-314, 321, 323, and 324; SEQ ID NOs: 310, 312, 314, 321, 324, and 325; SEQ ID NOs: 310, 312, 314, 321, 324, and 332; SEQ ID NOs: 315-317 and 326-328: SEQ ID NOs: 318-320 and 329-331; or SEQ ID NOs: 378-383. In certain embodiments, the fusion protein comprises a heavy chain variable region comprising the amino acid sequences of: SEQ ID NOs: 310, 311, and/or 312; SEQ ID NOs: 312, 313, and/or 314; SEQ ID NOS: 310, 312, and/or 314; SEQ ID NOs: 315, 316, and/or 317; SEQ ID NOs: 318, 319, and/or 320; or SEQ ID NOs: 378, 379, and/or 380. In certain embodiments, the fusion protein comprises a light chain variable region comprising the amino acid sequences of: SEQ ID NOs: 321, 322, and/or 323; SEQ ID NOs: 321, 323, and/or 324; SEQ ID NOs: 321, 324, and/or 325; SEQ ID NOs: 326, 327, and/or 328; SEQ ID NOs: 329, 330, and/or 331: SEQ ID NOs: 321, 324, and/or 332; or SEQ ID NOs: 381, 382, and/or 383. In certain embodiments, the fusion protein comprises: a heavy chain variable region comprising the amino acid sequences of SEQ ID NOs: 310, 311, and/or 312 and a light chain variable region comprising the amino acid sequences of SEQ ID NOs: 321, 322, and/or 323; a heavy chain variable region comprising the amino acid sequences of SEQ ID NOs: 312, 313, and/or 314 and a light chain variable region comprising the amino acid sequences of SEQ ID NOs: 321, 323, and/or 324; a heavy chain variable region comprising the amino acid sequences of SEQ ID NOs: 310, 312, and/or 314 and a light chain variable region comprising the amino acid sequences of SEQ ID NOs: 321, 324, and/or 325; a heavy chain variable region comprising the amino acid sequences of SEQ ID NOs: 315, 316, and/or 317 and a light chain variable region comprising the amino acid sequences of SEQ ID NOs: 326, 327, and/or 328: a heavy chain variable region comprising the amino acid sequences of SEQ ID NOs: 318, 319, and/or 320 and a light chain variable region comprising the amino acid sequences of SEQ ID NOs: 329, 330, and/or 331; a heavy chain variable region comprising the amino acid sequences of SEQ ID NOs: 310, 312, and/or 315 and a light chain variable region comprising the amino acid sequences of SEQ ID NOs: 321, 324, and/or 332; or a heavy chain variable region comprising the amino acid sequences of SEQ ID NOs: 378, 379, and/or 380 and a light chain variable region comprising the amino acid sequences of SEQ ID NOs: 381, 382, and/or 383. In each of the aforementioned embodiments, the CDRs may instead comprise amino acid sequences having at least 80%, at least 85%, at least 90%, at least 98%, or at least 99% sequence identity with the recited sequences.

In certain embodiments, the fusion protein comprises; three heavy chain CDRs, the CDRs comprising, respectively, amino acid sequences of SEQ ID NOs: 378, 379, and 380, or sequences having at least 80%, at least 85%, at least 90%, at least 95%, at least 98%, at least 99%, or 100% sequence identity therewith; and three light chain CDRs, the CDRs comprising, respectively, amino acid sequences of SEQ ID NOs: 381, 382, and 383, or sequences having at least 80%, at least 85%, at least 90%, at least 95%, at least 98%, at least 99%, or 100% sequence identity therewith.

In certain embodiments, the fusion protein comprises: three heavy chain CDRs, the CDRs comprising, respectively, amino acid sequences of SEQ ID NOs: 378, 379, and 380; and three light chain CDRs, the CDRs comprising, respectively, amino acid sequences of SEQ ID NOs: 381, 382, and 383.

In certain embodiments, the fusion protein comprises a heavy chain comprising an amino acid sequence of SEQ ID NO: 386, or a sequence having at least 80%, at least 85%, at least 90%, at least 95%, at least 98%, at least 99%, or 100% sequence identity therewith, and a light chain comprising an amino acid sequence of SEQ ID NO: 387, or a sequence having at least 80%, at least 85%, at least 90%, at least 95%, at least 98%, at least 99%, or 100% sequence identity therewith. In certain embodiments, the fusion protein comprises a heavy chain comprising and amino acid sequence of SEQ ID NO: 386 and a light chain comprising an amino acid sequence of SEQ ID NO: 387.

In some embodiments of the present invention, the fusion protein comprises an anti-PD-1 moiety that is selected or derived from a full-length, fragment, or variant of an antibody from Table 1.

In some embodiments, the fusion protein comprises an anti-PD-1 moiety that is selected or derived from a full-length, fragment, or variant of pembrolizumab (Keytruda®), nivolumab (Opdivo®), zimberelimab (WBP3055), or cetrelimab (JNJ-63723283).

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 12 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 280.

In some embodiments, the fusion protein comprises a sequence as shown in SEQ ID NO: 12 and a sequence as shown in SEQ ID NO: 280.

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 12 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 281.

In some embodiments, the fusion protein comprises a sequence as shown in SEQ ID NO: 12 and a sequence as shown in SEQ ID NO: 281.

In some embodiments, the fusion protein comprises a variable region of heavy chain (V_{H}) comprising a polypeptide sequence as shown in SEQ ID NO: 7 and a variable region of light chain (V_{L}) comprising a polypeptide sequence as shown in SEQ ID NO: 13. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 7 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 13.

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 13 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 282.

In some embodiments, the fusion protein comprises a sequence as shown in SEQ ID NO: 13 and a sequence as shown in SEQ ID NO: 282.

In some embodiments, the fusion protein comprises a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 13 and a sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 283.

In some embodiments, the fusion protein comprises a sequence as shown in SEQ ID NO: 13 and a sequence as shown in SEQ ID NO: 283.

In some embodiments, the fusion protein comprises a variable region of heavy chain (V_{H}) comprising a polypeptide sequence as shown in SEQ ID NO: 1. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 1.

In some embodiments, the fusion protein comprises a variable region of heavy chain (V_{H}) comprising a polypeptide sequence as shown in SEQ ID NO: 2. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 2.

In some embodiments, the fusion protein comprises a variable region of heavy chain (V_{H}) comprising a polypeptide sequence as shown in SEQ ID NO: 3. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 3.

In some embodiments, the fusion protein comprises a variable region of heavy chain (V_{H}) comprising a polypeptide sequence as shown in SEQ ID NO: 4. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 4.

In some embodiments, the fusion protein comprises a variable region of heavy chain (V_{H}) comprising a polypeptide sequence as shown in SEQ ID NO: 5. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 5.

In some embodiments, the fusion protein comprises a variable region of heavy chain (V_{H}) comprising a polypeptide sequence as shown in SEQ ID NO: 6. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 6.

In some embodiments, the fusion protein comprises a variable region of heavy chain (V_{H}) comprising a polypeptide sequence as shown in SEQ ID NO: 7. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 7.

In some embodiments, the fusion protein comprises a variable region of heavy chain (V_{H}) comprising a polypeptide sequence as shown in SEQ ID NO: 149. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 149.

In some embodiments, the fusion protein comprises a variable region of heavy chain (V_{H}) comprising a polypeptide sequence as shown in SEQ ID NO: 150. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 150.

In some embodiments, the fusion protein comprises a variable region of heavy chain (V_{H}) comprising a polypeptide sequence as shown in SEQ ID NO: 151. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 151.

In some embodiments, the fusion protein comprises a variable region of heavy chain (V_{H}) comprising a polypeptide sequence as shown in SEQ ID NO: 152. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 152.

In some embodiments, the fusion protein comprises a variable region of heavy chain (V_{H}) comprising a polypeptide sequence as shown in SEQ ID NO: 153. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 153.

In some embodiments, the fusion protein comprises a variable region of heavy chain (V_{H}) comprising a polypeptide sequence as shown in SEQ ID NO: 154. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 154.

In some embodiments, the fusion protein comprises a variable region of heavy chain (V_{H}) comprising a polypeptide sequence as shown in SEQ ID NO: 155. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 155.

In some embodiments, the fusion protein comprises a variable region of heavy chain (V_{H}) comprising a polypeptide sequence as shown in SEQ ID NO: 156. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 156.

In some embodiments, the fusion protein comprises a variable region of heavy chain (V_{H}) comprising a polypeptide sequence as shown in SEQ ID NO: 157. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 157.

In some embodiments, the fusion protein comprises a variable region of heavy chain (V_{H}) comprising a polypeptide sequence as shown in SEQ ID NO: 158. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 158.

In some embodiments, the fusion protein comprises a variable region of heavy chain (V_{H}) comprising a polypeptide sequence as shown in SEQ ID NO: 159. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 159.

In some embodiments, the fusion protein comprises a variable region of heavy chain (V_{H}) comprising a polypeptide sequence as shown in SEQ ID NO: 160. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 160.

In some embodiments, the fusion protein comprises a variable region of heavy chain (V_{H}) comprising a polypeptide sequence as shown in SEQ ID NO: 161. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 161.

In some embodiments, the fusion protein comprises a variable region of heavy chain (V_{H}) comprising a polypeptide sequence as shown in SEQ ID NO: 162. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 162.

In some embodiments, the fusion protein comprises a variable region of heavy chain (V_{H}) comprising a polypeptide sequence as shown in SEQ ID NO: 163. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 163.

In some embodiments, the fusion protein comprises a variable region of heavy chain (V_{H}) comprising a polypeptide sequence as shown in SEQ ID NO: 164. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 164.

In some embodiments, the fusion protein comprises a variable region of light chain (V_{L}) comprising a polypeptide sequence as shown in SEQ ID NO: 8. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 8.

In some embodiments, the fusion protein comprises a variable region of light chain (V_{L}) comprising a polypeptide sequence as shown in SEQ ID NO: 9. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 9.

In some embodiments, the fusion protein comprises a variable region of light chain (V_{L}) comprising a polypeptide sequence as shown in SEQ ID NO: 10. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 10.

In some embodiments, the fusion protein comprises a variable region of light chain (V_{L}) comprising a polypeptide sequence as shown in SEQ ID NO: 11. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 11.

In some embodiments, the fusion protein comprises a variable region of light chain (V_{L}) comprising a polypeptide sequence as shown in SEQ ID NO: 12. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 12.

In some embodiments, the fusion protein comprises a variable region of light chain (V_{L}) comprising a polypeptide sequence as shown in SEQ ID NO: 13. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 13.

In some embodiments, the fusion protein comprises a variable region of light chain (V_{L}) comprising a polypeptide sequence as shown in SEQ ID NO: 148. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 148.

In some embodiments, the fusion protein comprises a polypeptide sequence as shown in SEQ ID NO: 15. In some embodiments, the fusion protein comprises a polypeptide sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 15.

In some embodiments, the fusion protein comprises a polypeptide sequence as shown in SEQ ID NO: 280. In some embodiments, the fusion protein comprises a polypeptide sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 280.

In some embodiments, the fusion protein comprises a polypeptide sequence as shown in SEQ ID NO: 281. In some embodiments, the fusion protein comprises a polypeptide sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 281.

In some embodiments, the fusion protein comprises a polypeptide sequence as shown in SEQ ID NO: 282. In some embodiments, the fusion protein comprises a polypeptide sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 282.

In some embodiments, the fusion protein comprises a polypeptide sequence as shown in SEQ ID NO: 283. In some embodiments, the fusion protein comprises a polypeptide sequence that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 283.

In some embodiments, the fusion protein comprises a variable region of heavy chain (V_{H}) comprising a polypeptide sequence as shown in SEQ ID NO: 5 and a variable region of light chain (V_{L}) comprising a polypeptide sequence as shown in SEQ ID NO: 8. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 5 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 8.

In some embodiments, the fusion protein comprises a variable region of heavy chain (V_{H}) comprising a polypeptide sequence as shown in SEQ ID NO: 149 and a variable region of light chain (V_{L}) comprising a polypeptide sequence as shown in SEQ ID NO: 8. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 149 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 8.

In some embodiments, the fusion protein comprises a variable region of heavy chain (V_{H}) comprising a polypeptide sequence as shown in SEQ ID NO: 157 and a variable region of light chain (V_{L}) comprising a polypeptide sequence as shown in SEQ ID NO: 8. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 157 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 8.

In some embodiments, the fusion protein comprises a variable region of heavy chain (V_{H}) comprising a polypeptide sequence as shown in SEQ ID NO: 158 and a variable region of light chain (V_{L}) comprising a polypeptide sequence as shown in SEQ ID NO: 8. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 158 and a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 8.

In some embodiments, the fusion protein comprises a variable region of heavy chain (V_{H}) comprising a polypeptide sequence as shown in SEQ ID NO: 5. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 5.

In some embodiments, the fusion protein comprises a variable region of heavy chain (V_{H}) comprising a polypeptide sequence as shown in SEQ ID NO: 149. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 149.

In some embodiments, the fusion protein comprises a variable region of heavy chain (V_{H}) comprising a polypeptide sequence as shown in SEQ ID NO: 157. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 157.

In some embodiments, the fusion protein comprises a variable region of heavy chain (V_{H}) comprising a polypeptide sequence as shown in SEQ ID NO: 158. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 158.

In some embodiments, the fusion protein comprises a variable region of heavy chain (V_{H}) comprising a polypeptide sequence as shown in SEQ ID NO: 158. In some embodiments, the fusion protein comprises a variable region of heavy chain (V_{H}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 158.

In some embodiments, the fusion protein comprises a variable region of light chain (V_{L}) comprising a polypeptide sequence as shown in SEQ ID NO: 8. In some embodiments, the fusion protein comprises a variable region of light chain (V_{L}) that is at least 50%, at least 55%, at least 60%, at least 65%, at least 70%, at least 75%, at least 80%, at least 85%, at least 90%, at least 95%, at least 99%, at least 99.5%, or 100% identical to a sequence as shown in SEQ ID NO: 8.

### Linkers

In some embodiments, the linker comprises one or more polypeptide sequences having at least 80% sequence identity to any one of SEQ ID NOs: 17-34. In some embodiments, the linker can be a flexible linker. Flexible linkers can be applied when a joined domain requires a certain degree of movement or interaction. Flexible linkers can be composed of small, non-polar (e.g., Gly) or polar (e.g., Ser or Thr) amino acids. A flexible linker can have sequences consisting primarily of stretches of Gly and Ser residues (“GS” linker). A non-limiting example of a flexible linker can have the sequence of (Gly-Gly-Gly-Gly-Ser) n, wherein n can be 1, 2, 3, 4, 5, 6, 7, 8, 9, or 10 (SEQ ID NO: 32). By adjusting the copy number “n,” the length of this exemplary GS linker can be optimized to achieve appropriate separation of functional domains, or to maintain necessary inter-domain interactions. Besides GS linkers, other flexible linkers can be utilized for recombinant fusion proteins. In some embodiment, a flexible linker can have the sequence of (Gly) n, wherein n can be 6, 7, or 8 (SEQ ID NO: 33). In some cases, flexible linkers can also be rich in small or polar amino acids such as Gly and Ser, but can contain additional amino acids such as Thr and Ala to maintain flexibility. In some cases, the linker described herein can be a rigid linker. A rigid linker can be utilized to maintain a fixed distance between domains of the fusion protein or a fragment or a variant thereof described herein. Non-limiting examples of rigid linkers can be: alpha helix-forming linkers, pro-rich sequence, (XP) n, X-Pro backbone, (EAAAK) n (n=1-6) (SEQ ID NO: 34). Rigid linkers can exhibit relatively stiff structures by adopting α-helical structures or by containing multiple Pro residues in some cases. In some embodiments, the immune checkpoint inhibitor, such as a PD-1 inhibitor, and the cytokine trap (e.g., TGF-β trap) that can neutralize the cytokine (e.g., TGF-β) in a fusion protein or a fragment or a variant thereof described herein can be separated by an intervening sequence encoding an intervening linker polypeptide. In some embodiments, the immune checkpoint inhibitor, such as a PD-1 inhibitor, and the ADA2 (or mutants thereof) in a fusion protein or a fragment or a variant thereof described herein can be separated by an intervening sequence encoding an intervening linker polypeptide. In certain embodiments, the linker polypeptide comprises disclosed in the table below:

### Table 2. Linker Amino Acid Sequences and Polynucleotide Sequences

TABLE 2

| SEQ ID | |
|---|---|
| NO | Linkers |
| 17 | DPGGGGSGGGGSNPGS |
| 18 | GGGGSGGGGSGSDPGS |
| 19 | DPGSGGGGSGGGGSGS |
| 20 | GGGGSGGGGSGGGGSDPGS |
| 21 | DPGSGGGGSGGGGSGGGGS |
| 22 | DPGSGSVPLGSGSNPGS |
| 23 | DPGSGGSVPLGSGGSNPGS |
| 24 | DPGVLEREDKPTTSKPNPGS |
| 25 | DPGVLEREDVPTTSYPNPGS |
| 26 | DPGVLEREDKVTTSKYNPGS |
| 27 | DPVLEREDKVTTSKNPGS |
| 28 | DIEGRMD |
| 29 | GEGKSSGSGSESKAS |
| 30 | GSTSGSGKPGSGEGSTKG |
| 31 | A(EAAAK)₄ALEA(EAAAK)₄A |
| 32 | (G4S)n, n = 1-10 |
| 33 | (Gly)n, n = 6-8 |
| 34 | (EAAAK)n, n = 1-6 |

In some embodiments, the linker can be a flexible linker, a rigid linker, an in vivo cleavable linker, or any combination thereof. In some cases, the linker can connect functional domains together (as in flexible and rigid linkers) or releasing free functional domain in vivo as in in vivo cleavable linkers. In some embodiments, linkers can improve biological activity, increase expression yield, and achieving desirable pharmacokinetic profiles. In some embodiments, the linker can also comprise hydrazone, peptide, disulfide, or thioesther.

In some cases, the linker sequence described herein can include a flexible linker. Flexible linkers can be applied when a joined domain requires a certain degree of movement or interaction. Flexible linkers can be composed of small, non-polar (e.g., Gly) or polar (e.g., Ser or Thr) amino acids. A flexible linker can have sequences consisting primarily of stretches of Gly and Ser residues (“GS” linker). An example of a flexible linker can have the sequence of (G4S) n, wherein n is 1, 2, 3, 4, 5, 6, 7, 8, 9 or 10 (SEQ ID NO: 32). In some embodiment, a flexible linker can have the sequence of (Gly) n, wherein n can be 6, 7, or 8 (SEQ ID NO: 33). In some cases, flexible linkers can also be rich in small or polar amino acids such as Gly and Ser, but can contain additional amino acids such as Thr and Ala to maintain flexibility. In other cases, polar amino acids such as Lys and Glu can be used to improve solubility. By adjusting the copy number “n,” the length of these non-limiting exemplary linkers can be optimized to achieve appropriate separation of functional domains, or to maintain necessary inter-domain interactions. Besides GS linkers, other flexible linkers can be utilized for the fusion protein or a fragment or a variant thereof described herein. In some cases, flexible linkers can also be rich in small or polar amino acids such as Gly and Ser, but can contain additional amino acids such as Thr and Ala to maintain flexibility. In other cases, polar amino acids such as Lys and Glu can be used to improve solubility.

Flexible linkers included in linker sequences described herein, can be rich in small or polar amino acids such as Gly and Ser to provide good flexibility and solubility. Flexible linkers can be suitable choices when certain movements or interactions are desired for fusion protein or a fragment or a variant thereof domains. In addition, although flexible linkers do not have rigid structures, they can serve as a passive linker to keep a distance between functional domains. The length of flexible linkers can be adjusted to allow for proper folding or to achieve optimal biological activity of the fusion protein or a fragment or a variant thereof.

The linker described herein can further include a rigid linker in some cases. A rigid linker can be utilized to maintain a fixed distance between domains of the fusion protein or a fragment or a variant thereof described herein. Examples of rigid linkers can be: alpha helix-forming linkers, pro-rich sequence, (XP) n, X-Pro backbone, (EAAAK) n (n=1-6) (SEQ ID NO: 34), to name a few. Rigid linkers can exhibit relatively stiff structures by adopting α-helical structures or by containing multiple Pro residues in some cases.

In some embodiments, the linker described herein can be a cleavable linker. In other cases a linker is not cleavable. Linkers that are not cleavable can covalently join functional domains of the fusion protein or a fragment or a variant thereof together to act as one molecule throughout an in vivo processes or an ex vivo process. A linker can also be cleavable in vivo. A cleavable linker can be introduced to release free functional domains in vivo. A cleavable linker can be cleaved by the presence of reducing reagents, proteases, to name a few. For example, a reduction of a disulfide bond can be utilized to produce a cleavable linker. In the case of a disulfide linker, a cleavage event through disulfide exchange with a thiol, such as glutathione, could produce a cleavage. In other cases, an in vivo cleavage of a linker in a recombinant fusion protein can also be carried out by proteases that can be expressed in vivo under pathological conditions (e.g. cancer or inflammation), in specific cells or tissues, or constrained within certain cellular compartments. In some cases, a cleavable linker can allow for targeted cleavage. For example, the specificity of many proteases can offer slower cleavage of a linker in constrained compartments. A cleavable linker can also comprise hydrazone, peptides, disulfide, or thioesther. For example, a hydrazone can confer serum stability. In other cases, a hydrazone can allow for cleavage in an acidic compartment. An acidic compartment can have a pH up to 7. A linker can also include a thioether. A thioether can be nonreducible. A thioether can be designed for intracellular proteolytic degradation.

In some cases, a linker can be engineered linker. For example, a linker can be designed to comprise chemical characteristics such as hydrophobicity. In some cases, at least two different linker polypeptide sequences can encode the same polypeptide linker sequence. Methods of designing linkers can be computational. In some cases, computational methods can include graphic techniques. Computation methods can be used to search for suitable peptides from libraries of three-dimensional peptide structures derived from databases. For example, a Brookhaven Protein Data Bank (PDB) can be used to span the distance in space between selected amino acids of a linker. In some cases, a polypeptide linker can also include one or more GS linker sequences, for instance, (G4S) n (SEQ ID NO: 556), (GS) n (SEQ ID NO: 557), (SG) n (SEQ ID NO: 558), (GSG) n (SEQ ID NO: 559), and (SGSG) n (SEQ ID NO: 560), wherein n can be any number from zero to fifteen.

### Methods of Treating Cancer

Also provided herein is a method of treating cancer with the fusion protein of the present invention. The development of monoclonal antibodies targeting blockade of an immune checkpoint pathway, such as PD-1 and PD-L1/2 signaling pathway and CTLA-4 and CD80/86 signaling pathway, have revolutionized cancer treatment demonstrating durable clinical activity in several cancer indications including, but not limited to, melanoma (e.g., advanced or metastatic melanoma), lung cancer (e.g., non-small cell lung cancer and small cell lung cancer), breast cancer (e.g., HER-2 negative breast cancer, estrogen-receptor+/HER-2-breast cancer, and triple negative breast cancer), pancreatic cancer (e.g., pancreatic adenocarcinoma), renal cell carcinoma, bladder cancer, head and neck squamous cell carcinoma, MSI-high colorectal carcinoma, Merkel cell carcinoma, and Hodgkin lymphoma, as well as gastric, head and neck, prostate, gynecologic, and hematologic cancers. Despite the durable responses, the response rate remains very low, and several patients have developed resistance leading to disease progression. Furthermore, in several indications, such as ovarian, gastroesophageal, prostate, pancreas and many other cancers, checkpoint inhibitors have failed to show any substantive clinical responses.

The failure of checkpoint blockade can be attributed to the complexity of the immunosuppressive factors present in the tumor microenvironment. These factors can include, but not limited to, suppressive cells, such as myeloid derived suppressive cells, tumor-associated macrophages (TAM); suppressive cytokine and growth factors, such as TGF-β and interleukin 10 (IL-10); and metabolic derivative, such as adenosine and indoleamine 2,3-dioxygenase (IDO) byproducts. Anti-PD-1-TGFβRII fusion protein provided herein is an example of a therapy that can target two negative suppressive pathways in the tumor microenvironment. These pathways can include cell intrinsic interactions mediated by tumor cells to immune, in which PD-1/PD-L1 interaction can play a major role, and cell extrinsic interactions mediated by immunosuppressive cytokines, of which TGF-β can be a prominent member.

In some embodiments, the cancer is, but not limited to, glioblastoma, colorectal, gastric, cervical, ovarian, pancreatic, prostate, breast, and renal cancers. In addition, the fusion proteins as described herein can be amenable to indications, such as in non-small cell cancer (NSCL) and melanoma, in which response rate of checkpoint blockades is lower and TGF-β is highly expressed.

Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, triple negative breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, hepatocellular carcinoma (HCC), choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pincaloma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer whose phenotype is determined by the method of the present disclosure is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, brenner, or undifferentiated. In some embodiments, the present disclosure is used in the treatment, diagnosis, and/or prognosis of lymphoma or its subtypes, including, but not limited to, mantle cell lymphoma.

In certain embodiments, anti-PD-1-TGFβRII fusion protein provided herein is an example of a therapy that can be used to treat cancers that have about 0, 5, 10, 15, 20, 25, 30, 35, 40, 45, 50, 55, 60, 65, 70, 75, 80, 85, 90 or 95% average response rate to standard therapy (including but not limited to chemotherapy, chemotherapy and current clinical trial therapies). In certain embodiments, anti-PD-1-ADA2 fusion protein provided herein is an example of a therapy that can be used to treat cancers that have about 0, 5, 10, 15, 20, 25, 30, 35, 40, 45, 50, 55, 60, 65, 70, 75, 80, 85, 90 or 95% average response rate to standard therapy (including but not limited to chemotherapy, chemotherapy and current clinical trial therapies). Such cancers include but are not limited to, Hodgkin's lymphoma, melanoma, non-small cell lung cancer (NSCLC), microsatellite instability (MSI)-high or mismatch repair (MMR)-deficient solid tumors, CSCC, RCC, CRC, melanoma, Merkel cell cancer, bladder cancer, RCC, hepatocellular carcinoma (HCC), head & neck cancer (H&N), cervical cancer, gastric cancer, small cell lung cancer (SCLC), endometrial cancer, mesothelioma, ovarian cancer, triple negative breast cancer (TNBC), breast cancer, colorectal cancer (CRC), pancreatic cancer, prostate cancer.

### Combination Therapy

In some embodiments, the fusion protein is administered as a combination therapy with an additional therapeutic agent. In some embodiments, an additional therapeutic agent comprises a biological molecule, such as an antibody. For example, treatment can involve the combined administration of the fusion protein with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX). In some embodiments, an additional therapeutic agent comprises an agent such as a small molecule. For example, treatment can involve the combined administration of the fusion protein or a fragment or a variant thereof provided herein with a small molecule that acts as an inhibitor against tumor-associated antigens including, but not limited to, EGFR, HER2 (ErbB2), and/or VEGF. In some embodiments, the fusion protein or a fragment or a variant thereof is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor. In another embodiment, the additional therapeutic agent is chemotherapy or other inhibitors that reduce the number of TREG cells. In certain embodiments, the therapeutic agent is cyclophosphamide or an anti-CTLA4 antibody. In another embodiment, the additional therapeutic reduces the presence of myeloid-derived suppressor cells. In a further embodiment, the additional therapeutic is carbotaxol. In a further embodiment, the additional therapeutic agent is ibrutinib.

In some embodiments, the method can further comprise one or more checkpoint inhibitors in combination with the fusion protein or a fragment or a variant thereof as described herein. In some embodiments, the additional checkpoint inhibitor can be an anti-CTLA-4 antibody. The anti-CTLA-4 antibody (e.g., ipilimumab) has shown durable anti-tumor activities and prolonged survival in participants with advanced melanoma, resulting in its Food and Drug Administration (FDA) approval in 2011. See Hodi et al., Improved survival with ipilimumab in patients with metastatic melanoma. N Engl J Med. (2010) August 19; 363 (8): 711-23. In some embodiments, the one or more checkpoint inhibitors can be an anti-PD-L1 antibody. In some embodiments, the anti-PD-L1 antibody can be a full length atezolizumab (anti-PD-L1), avelumab (anti-PD-L1), durvalumab (anti-PD-L1), or a fragment or a variant thereof. In some embodiments, the one or more checkpoint inhibitors can be any one or more of CD27 inhibitor, CD28 inhibitor, CD40 inhibitor, CD122 inhibitor, CD137 inhibitor, OX40 (also known as CD134) inhibitor, GITR inhibitor, ICOS inhibitor, or any combination thereof. In some embodiments, the one or more checkpoint inhibitors can be any one or more of A2AR inhibitor, B7-H3 (also known as CD276) inhibitor, B7-H4 (also known as VTCN1) inhibitor, BTLA inhibitor, IDO inhibitor, KIR inhibitor, LAG3 inhibitor, TIM-3 inhibitor, VISTA inhibitor, or any combination thereof.

In certain embodiments, an additional therapeutic agent comprises a second immunotherapeutic agent. In some embodiments, the additional immunotherapeutic agent includes, but is not limited to, a colony stimulating factor, an interleukin, an antibody that blocks immunosuppressive functions (e.g., an anti-CTLA-4 antibody, anti-CD28 antibody, anti-CD3 antibody, anti-PD-L1 antibody, anti-TIGIT antibody), an antibody that enhances immune cell functions (e.g., an anti-GITR antibody, an anti-OX-40 antibody, an anti-CD40 antibody, or an anti-4-1BB antibody), a toll-like receptor (e.g., TLR4, TLR7, TLR9), a soluble ligand (e.g., GITRL, GITRL-Fc, OX-40L, OX-40L-Fc, CD40L, CD40L-Fc, 4-1BB ligand, or 4-1BB ligand -Fc), or a member of the B7 family (e.g., CD80, CD86). In some embodiments, the additional immunotherapeutic agent targets CTLA-4, CD28, CD3, PD-L1, TIGIT, GITR, OX-40, CD-40, or 4-1BB.

In some embodiments, the additional therapeutic agent is an additional immune checkpoint inhibitor. In some embodiments, the additional immune checkpoint inhibitor is an anti-PD-L1 antibody, an anti-CTLA-4 antibody, an anti-CD28 antibody, an anti-TIGIT antibody, an anti-LAG3 antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments, the additional therapeutic agent is an anti-PD-L1 antibody selected from the group consisting of: BMS935559 (MDX-1105), atexolizumab (MPDL3280A), durvalumab (MEDI4736), and avelumab (MSB0010718C). In some embodiments, the additional therapeutic agent is an anti-CTLA-4 antibody selected from the group consisting of: ipilimumab (YERVOY) and tremelimumab. In some embodiments, the additional therapeutic agent is an anti-LAG-3 antibody selected from the group consisting of: BMS-986016 and LAG525. In some embodiments, the additional therapeutic agent is an anti-OX-40 antibody selected from the group consisting of: MEDI6469, MEDI0562, and MOXR0916. In some embodiments, the additional therapeutic agent is an anti-4-1BB antibody selected from the group consisting of: PF-05082566. In some embodiments, the fusion protein or a fragment or a variant thereof can be administered in combination with a biologic molecule selected from the group consisting of: cytokines, adrenomedullin (AM), angiopoietin (Ang), BMPs, BDNF, EGF, erythropoietin (EPO), FGF, GDNF, granulocyte colony stimulating factor (G-CSF), granulocyte-macrophage colony stimulating factor (GM-CSF), macrophage colony stimulating factor (M-CSF), stem cell factor (SCF), GDF9, HGF, HDGF, IGF, migration-stimulating factor, myostatin (GDF-8), NGF, neurotrophins, PDGF, thrombopoietin, TGF-α, TGF-β, TNF-α, VEGF, PIGF, gamma-IFN, IL-1, IL-2, IL-3, IL-4, IL-5, IL-6, IL-7, IL-12, IL-15, and IL-18.

### Cytokines

In some cases, the cytokine comprises at least one chemokine, interferon, interleukin, lymphokine, tumor necrosis factor, or variant or combination thereof. In some cases, the cytokine is an interleukin. In some cases the interleukin is at least one of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33 and functional variants and fragments thereof. In some embodiments, the cytokines can be membrane bound or secreted. In embodiments, the cytokine is soluble IL-15, soluble IL-15/IL-15Ra complex (e.g., ALT-803). In certain cases, the interleukin can comprise membrane bound IL-15 (mbIL-15) or a fusion of IL-15 and IL-15Rα. In some embodiments, a mbIL-15 is a membrane-bound chimeric IL-15 which can be co-expressed with a modified immune effector cell described herein. In some embodiments, the mbIL-15 comprises a full-length IL-15 (e.g., a native IL-15 polypeptide) or fragment or variant thereof, fused in frame with a full length IL-15Rα, fragment or variant thereof. In some cases, the IL-15 is indirectly linked to the IL-15Ra through a linker. In some instances, the mbIL-15 is as described in Hurton et al., “Tethered IL-15 augments antitumor activity and promotes a stem-cell memory subset in tumor-specific T cells,” PNAS 2016. In some cases, the cytokine is expressed in the same immune effector cell as the CAR.

In some embodiments, the mbIL-15 is expressed with a cell tag such as HERIt, HER-It-1, CD20t-1 or CD20 as described herein. The mbIL-15 can be expressed in-frame with HER It, HER-1t-1, CD20t-1 or CD20.

In some embodiments, the mbIL-15 can be under the control of an inducible promoter for gene transcription. In one aspect, the inducible promoter can be a gene switch ligand inducible promoter. In some cases, an inducible promoter can be a small molecule ligand -inducible two polypeptide ecdysone receptor-based gene switch, such as RHEOSWITCH® gene switch.

In another aspect, the interleukin can comprise IL-12. In some embodiments, the IL-12 is a single chain IL-12 (scIL-12), protease sensitive IL-12, destabilized IL-12, membrane bound IL-12, intercalated IL-12. In some instances, the IL-12 variants are as described in WO2015/095249, WO2016/048903, WO2017/062953, all of which is incorporated by reference in their entireties. In some embodiments, the cytokines described above can be under the control of an inducible promoter for gene transcription. In one aspect, the inducible promoter can be a gene switch ligand inducible promoter. In some cases, an inducible promoter can be a small molecule ligand -inducible two polypeptide ecdysone receptor-based gene switch, such as RHEOSWITCH® gene switch.

In some embodiments, the fusion protein as described herein can be under the control of an inducible promoter for gene transcription. In one aspect, the inducible promoter can be a gene switch ligand inducible promoter. In some cases, an inducible promoter can be a small molecule ligand -inducible two polypeptide ecdysone receptor-based gene switch, such as RHEOSWITCH® gene switch.

### Gene Switches

Provided herein are gene switch polypeptides, polynucleotides encoding ligand -inducible gene switch polypeptides, and methods and systems incorporating these polypeptides and/or polynucleotides. The term “gene switch” refers to the combination of a response element associated with a promoter, and for instance, an ecdysone receptor (EcR) based system which, in the presence of one or more ligands, modulates the expression of a gene into which the response element and promoter are incorporated. Tightly regulated inducible gene expression systems or gene switches are useful for various applications such as gene therapy, large scale production of proteins in cells, cell based high throughput screening assays, functional genomics and regulation of traits in transgenic plants and animals. Such inducible gene expression systems can include ligand inducible heterologous gene expression systems.

An early version of EcR-based gene switch used Drosophila melanogaster EcR (DmEcR) and Mus musculus RXR (MmRXR) polypeptides and showed that these receptors in the presence of steroid, ponasteroneA, transactivate reporter genes in mammalian cell lines and transgenic mice (Christopherson et al., 1992; No et al., 1996). Later, Suhr et al., 1998 showed that non-steroidal ecdysone agonist, tebufenozide, induced high level of transactivation of reporter genes in mammalian cells through Bombyx mori EcR (BmEcR) in the absence of exogenous heterodimer partner.

International Patent Applications No. PCT/US97/05330 (WO 97/38117) and PCT/US99/08381 (WO99/58155) disclose methods for modulating the expression of an exogenous gene in which a DNA construct comprising the exogenous gene and an ecdysone response element is activated by a second DNA construct comprising an ecdysone receptor that, in the presence of a ligand therefor, and optionally in the presence of a receptor capable of acting as a silent partner, binds to the ecdysone response element to induce gene expression. In this example, the ecdysone receptor was isolated from Drosophila melanogaster. Typically, such systems require the presence of the silent partner, preferably retinoid X receptor (RXR), in order to provide optimum activation. In mammalian cells, insect ecdysone receptor (EcR) is capable of heterodimerizing with mammalian retinoid X receptor (RXR) and, thereby, be used to regulate expression of target genes or heterologous genes in a ligand dependent manner. International Patent Application No. PCT/US98/14215 (WO 99/02683) discloses that the ecdysone receptor isolated from the silk moth Bombyx mori is functional in mammalian systems without the need for an exogenous dimer partner.

U.S. Pat. No. 6,265,173 discloses that various members of the steroid/thyroid superfamily of receptors can combine with Drosophila melanogaster ultraspiracle receptor (USP) or fragments thereof comprising at least the dimerization domain of USP for use in a gene expression system. U.S. Pat. No. 5,880,333 discloses a Drosophila melanogaster EcR and ultraspiracle (USP) heterodimer system used in plants in which the transactivation domain and the DNA binding domain are positioned on two different hybrid proteins. In each of these cases, the transactivation domain and the DNA binding domain (either as native EcR as in International Patent Application No. PCT/US98/14215 or as modified EcR as in International Patent Application No. PCT/US97/05330) were incorporated into a single molecule and the other heterodimeric partners, either USP or RXR, were used in their native state.

International Patent Application No. PCT/US01/0905 discloses an ecdysone receptor-based inducible gene expression system in which the transactivation and DNA binding domains are separated from each other by placing them on two different proteins results in greatly reduced background activity in the absence of a ligand and significantly increased activity over background in the presence of a ligand. This two-hybrid system is a significantly improved inducible gene expression modulation system compared to the two systems disclosed in applications PCT/US97/05330 and PCT/US98/14215. The two-hybrid system is believed to exploit the ability of a pair of interacting proteins to bring the transcription activation domain into a more favorable position relative to the DNA binding domain such that when the DNA binding domain binds to the DNA binding site on the gene, the transactivation domain more effectively activates the promoter (see, for example, U.S. Pat. No. 5,283,173). The two-hybrid gene expression system comprises two gene expression cassettes; the first encoding a DNA binding domain fused to a nuclear receptor polypeptide, and the second encoding a transactivation domain fused to a different nuclear receptor polypeptide. In the presence of ligand, it is believed that a conformational change is induced which promotes interaction of the antibody with the TGF-β cytokine trap, thereby resulting in dimerization of the DNA binding domain and the transactivation domain. Since the DNA binding and transactivation domains reside on two different molecules, the background activity in the absence of ligand is greatly reduced.

Certain modifications of the two-hybrid system could also provide improved sensitivity to non-steroidal ligands for example, diacylhydrazines, when compared to steroidal ligands for example, ponasterone A (“PonA”) or muristerone A (“MurA”). That is, when compared to steroids, the non-steroidal ligands provided higher gene transcription activity at a lower ligand concentration. Furthermore, the two-hybrid system avoids some side effects due to overexpression of RXR that can occur when unmodified RXR is used as a switching partner. In a preferred two-hybrid system, native DNA binding and transactivation domains of EcR or RXR are eliminated and as a result, these hybrid molecules have less chance of interacting with other steroid hormone receptors present in the cell, thereby resulting in reduced side effects.

The ecdysone receptor (EcR) is a member of the nuclear receptor superfamily and is classified into subfamily 1, group H (referred to herein as “Group H nuclear receptors”). The members of each group share 40-60% amino acid identity in the E (ligand binding) domain (Laudet et al., A Unified Nomenclature System for the Nuclear Receptor Subfamily, 1999; Cell 97:161-163). In addition to the ecdysone receptor, other members of this nuclear receptor subfamily 1, group H include: ubiquitous receptor (UR), Orphan receptor 1 (OR-1), steroid hormone nuclear receptor 1 (NER-1), RXR interacting protein-15 (RIP-15), liver x receptor β (LXRB), steroid hormone receptor like protein (RLD-1), liver x receptor (LXR), liver x receptor α (LXRa), farnesoid x receptor (FXR), receptor interacting protein 14 (RIP-14), and farnesol receptor (HRR-1).

In some cases, an inducible promoter (“IP”) can be a small molecule ligand -inducible two polypeptide ecdysone receptor-based gene switch, such as Intrexon Corporation's RHEOSWITCH® gene switch. In some cases, a gene switch can be selected from ecdysone-based receptor components as described in, but without limitation to, any of the systems described in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/US2002/005235 (WO 2002/066613); U.S. application Ser. No. 10/468,200 (U.S. Pub. No. 20120167239); PCT/US2002/005706 (WO 2002/066614); U.S. Pat. Nos. 7,531,326; 8,236,556; 8,598,409; PCT/US2002/005090 (WO 2002/066612); U.S. Pat. No. 8,715,959 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); U.S. application Ser. No. 12/241,018 (U.S. Pub. No. 20090136465); PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); WO 2018/132494 (PCT/US2018/013196); and U.S. Pat. No. 9,402,919; each of which is incorporated by reference in its entirety.

As used herein, the term “ligand,” as applied to ligand -activated ecdysone receptor-based gene switches are small molecules of varying solubility (such as diacylhydrazine compounds) having the capability of activating a gene switch to stimulate gene expression (i.e., therein providing ligand inducible expression of polynucleotides (e.g., mRNAs, miRNAs, etc) and/or polypeptides). Examples of such ligands include, but are not limited to those described in: WO 2004/072254 (PCT/US2004/003775); WO 2004/005478 (PCT/US2003/021149); WO 2005/017126 (PCT/US2004/005149); WO 2004/078924 (PCT/US2004/005912); WO 2008/153801 (PCT/US2008/006757); WO 2009/114201 (PCT/US2009/001639); WO 2013/036758 (PCT/US2012/054141); WO 2014/144380 (PCT/US2014/028768); and, WO 2016/044390 (PCT/US2015/050375); each of which are hereby incorporated by reference herein in the entirety.

Examples of ligands also include, without limitation, an ecdysteroid, such as ecdysone, 20-hydroxyecdysone, ponasterone A, muristerone A, and the like, 9-cis-retinoic acid, synthetic analogs of retinoic acid, N,N′-diacylhydrazines such as those disclosed in U.S. Pat. Nos. 6,258,603; 6,013,836; 5,117,057; 5,530,028; 5,378,726; 7,304,161; 7,851,220; 8,748,125; 9,272,986; 7,456,315; 7,563,928; 8,524,948; 9,102,648; 9,169,210; 9,255,273; and, 9,359,289; oxadiazolines as described in U.S. Pat. Nos. 8,669,072; and, 8,895,306; dibenzoylalkyl cyanohydrazines such as those disclosed in European Application No. 2,461,809; N-alkyl-N,N′-diaroylhydrazines such as those disclosed in U.S. Pat. No. 5,225,443; N-acyl-N-alkylcarbonylhydrazines such as those disclosed in European Application No. 234,994; N-aroyl-N-alkyl-N′-aroylhydrazines such as those described in U.S. Pat. No. 4,985,461; amidoketones such as those described in U.S. Pat. Nos. 7,375,093; 8,129,355; and, 9,802,936; each of which is incorporated herein by reference and other similar materials including 3,5-di-tert-butyl-4-hydroxy-N-isobutyl-benzamide, 8-O-acetylharpagide, oxysterols, 22 (R) hydroxycholesterol, 24 (S) hydroxycholesterol, 25-epoxycholesterol, T0901317, 5-alpha-6-alpha-epoxycholesterol-3-sulfate (ECHS), 7-ketocholesterol-3-sulfate, famesol, bile acids, 1,1-biphosphonate esters, juvenile hormone III, and the like. Examples of diacylhydrazine ligands useful in the present invention include RG-115819 (3,5-Dimethyl-benzoic acid N-(1-ethyl-2,2-dimethyl-propyl)-N′-(2-methyl-3-methoxybenzoyl)-hydrazide), RG-115932 ((R)-3,5-Dimethyl-benzoic acid N-(1-tert-butyl-butyl) N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide). See, e.g., WO 2008/153801 (PCT/US2008/006757); and, WO 2013/036758 (PCT/US2012/054141), both of which are incorporated herein by reference in their entireties.

For example, a ligand for the ecdysone receptor-based gene switch may be selected from any suitable ligands. Both naturally occurring ecdysone or ecdysone analogs (e.g., 20-hydroxyecdysone, muristerone A, ponasterone A, ponasterone B, ponasterone C, 26-iodoponasterone A, inokosterone or 26-mesylinokosterone) and non-steroid inducers may be used as a ligand for gene switch of the present invention. U.S. Pat. No. 6,379,945, describes an insect steroid receptor isolated from Heliothis virescens (“HEcR”) which is capable of acting as a gene switch responsive to both steroid and certain non-steroidal inducers. Non-steroidal inducers have a distinct advantage over steroids, in this and many other systems which are responsive to both steroids and non-steroid inducers, for several reasons including, for example: lower manufacturing cost, metabolic stability, absence from insects, plants, or mammals, and environmental acceptability. U.S. Pat. No. 6,379,945 describes the utility of two dibenzoylhydrazines, 1,2-dibenzoyl-1-tert-butyl-hydrazine and tebufenozide (N-(4-ethylbenzoyl)-N′-(3,5-dimethylbenzoyl)-N′-tert-butyl-hydrazine) as ligands for an ecdysone-based gene switch. Also included in the present invention as a ligand are other dibenzoylhydrazines, such as those disclosed in U.S. Pat. No. 5,117,057. Use of tebufenozide as a chemical ligand for the ecdysone receptor from Drosophila melanogaster is also disclosed in U.S. Pat. No. 6,147,282. Additional, non-limiting examples of ecdysone ligands are 3,5-di-tert-butyl-4-hydroxy-N-isobutyl-benzamide, 8-O-acetylharpagide, a 1,2-diacyl hydrazine, an N′-substituted-N,N′-disubstituted hydrazine, a dibenzoylalkyl cyanohydrazine, an N-substituted-N-alkyl-N,N-diaroyl hydrazine, an N-substituted-N-acyl-N-alkyl, carbonyl hydrazine or an N-aroyl-N′-alkylN′-aroyl hydrazine. (See U.S. Pat. No. 6,723,531).

In some embodiments, the ligand for an ecdysone-based gene switch system is a diacylhydrazine ligand or chiral diacylhydrazine ligand. The ligand used in the gene switch system may be compounds of Formula I

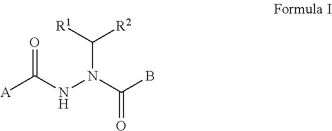

wherein A is alkoxy, arylalkyloxy or aryloxy; B is optionally substituted aryl or optionally substituted heteroaryl; and R1 and R2 are independently optionally substituted alkyl, arylalkyl, hydroxyalkyl, haloalkyl, optionally substituted cycloalkyl, optionally substituted alkenyl, optionally substituted alkynyl, optionally substituted heterocyclo, optionally substituted aryl or optionally substituted heteroaryl; or pharmaceutically acceptable salts, hydrates, crystalline forms or amorphous forms thereof.

In another embodiment, the ligand may be enantiomerically enriched compounds of Formula II

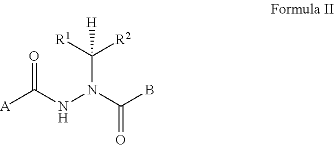

wherein A is alkoxy, arylalkyloxy, aryloxy, arylalkyl, optionally substituted aryl or optionally substituted heteroaryl; B is optionally substituted aryl or optionally substituted heteroaryl; and R1 and R2 are independently optionally substituted alkyl, arylalkyl, hydroxyalkyl, haloalkyl, optionally substituted cycloalkyl, optionally substituted alkenyl, optionally substituted alkynyl, optionally substituted heterocyclo, optionally substituted aryl or optionally substituted heteroaryl; with the proviso that R1 does not equal R2; wherein the absolute configuration at the asymmetric carbon atom bearing R1 and R2 is predominantly S; or pharmaceutically acceptable salts, hydrates, crystalline forms or amorphous forms thereof.

In certain embodiments, the ligand may be enantiomerically enriched compounds of Formula III

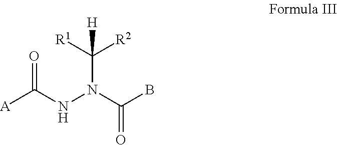

wherein A is alkoxy, arylalkyloxy, aryloxy, arylalkyl, optionally substituted aryl or optionally substituted heteroaryl; B is optionally substituted aryl or optionally substituted heteroaryl; and R1 and R2 are independently optionally substituted alkyl, arylalkyl, hydroxyalkyl, haloalkyl, optionally substituted cycloalkyl, optionally substituted alkenyl, optionally substituted alkynyl, optionally substituted heterocyclo, optionally substituted aryl or optionally substituted heteroaryl; with the proviso that RI does not equal R2; wherein the absolute configuration at the asymmetric carbon atom bearing R1 and R2 is predominantly R; or pharmaceutically acceptable salts, hydrates, crystalline forms or amorphous forms thereof.

In some embodiments, a ligand may be (R)-3,5-dimethyl-benzoic acid N-(1-tertbutyl-butyl)-N′-(2-ethyl-3-methoxy-benzoyl)-hydrazide having an enantiomeric excess of at least 95% or a pharmaceutically acceptable salt, hydrate, crystalline form or amorphous form thereof.

The diacylhydrazine ligands of Formula I and chiral diacylhydrazine ligands of Formula II or III, when used with an ecdysone-based gene switch system, provide the means for external temporal regulation of expression of a therapeutic polypeptide or therapeutic polynucleotide of the present invention. See U.S. Pat. Nos. 8,076,517; 8,884,060; and, 9,598,355; each of which are fully incorporated by reference herein.

The ligands used in the present invention may form salts. The term “salt(s)” as used herein denotes acidic and/or basic salts formed with inorganic and/or organic acids and bases. In addition, when a compound of Formula I, II or III contains both a basic moiety and an acidic moiety, zwitterions (“inner salts”) may be formed and are included within the term “salt(s)” as used herein. Pharmaceutically acceptable (i.e., non-toxic, physiologically acceptable) salts are used, although other salts are also useful, e.g., in isolation or purification steps which may be employed during preparation. Salts of the compounds of Formula I, II or III may be formed, for example, by reacting a compound with an amount of acid or base, such as an equivalent amount, in a medium such as one in which the salt precipitates or in an aqueous medium followed by lyophilization.

The ligands which contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The ligands which contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine and the like.

Non-limiting examples of the ligands for the inducible gene expression system also includes those utilizing the FK506 binding domain are FK506, Cyclosporin A, or Rapamycin. FK506, rapamycin, and their analogs are disclosed in U.S. Pat. Nos. 6,649,595; 6,187,757; 7,276,498; and, 7,273,874.

In some embodiments, a diacylhydrazine ligand for inducible gene expression is administered at unit daily dose of about 5, 10, 15, 20, 25, 30, 35, 40, 50, 60, 70, 80, 90, 100 or 120 mg. In some embodiments, the diacylhydrazine ligand is administered at a unit daily dose of about 5 mg. In some embodiments, the diacylhydrazine ligand is administered at a unit daily dose of about 10 mg. In some embodiments, the diacylhydrazine ligand is administered at a unit daily dose of about 15 mg. In some embodiments, the diacylhydrazine ligand is administered daily at a unit daily dose of about 20 mg.

In some embodiments, the combination therapy with two or more therapeutic agents can use agents that work by different mechanisms of action, although this is not required. Combination therapy using agents with different mechanisms of action can result in additive or synergetic effects. Combination therapy can allow for a lower dose of each agent than is used in monotherapy, thereby reducing toxic side effects and/or increasing the therapeutic index of the agent(s). Combination therapy can decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In certain embodiments, in addition to administering the fusion protein or a fragment or a variant thereof described herein, the method or treatment further comprises administering at least one additional therapeutic agent. An additional therapeutic agent can be administered prior to, concurrently with, and/or subsequently to, administration of the agent. In some embodiments, the at least one additional therapeutic agent comprises 1, 2, 3, or more additional therapeutic agents.

Therapeutic agents that can be administered in combination with the fusion protein or a fragment or a variant thereof described herein include chemotherapeutic agents. Thus, in some embodiments, the method or treatment involves the administration of an agent described herein in combination with a chemotherapeutic agent or in combination with a cocktail of chemotherapeutic agents. In some embodiments, the methods can further comprise one or more antineoplastic agents, such as cisplatin, capecitabine, or 5-fluorouracil, in combination with the fusion protein or a fragment or a variant thereof as described herein. Treatment with an agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4th Edition, 2008, M. C. Perry, Editor, Lippincott, Williams & Wilkins, Philadelphia, PA.

Useful classes of chemotherapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

The fusion protein or a fragment or a variant thereof provided herein can be used alone or in combination with conventional therapeutic regimens such as surgery, irradiation, chemotherapy and/or bone marrow transplantation (autologous, syngeneic, allogeneic or unrelated). A set of tumor antigens can be useful, e.g., in a large fraction of cancer patients.

Fusion Proteins in Combination with Chimeric Receptors

In some embodiments, the fusion protein is administered as a combination therapy with an additional therapeutic agent. In some embodiments, an additional therapeutic agent comprises a chimeric receptor, such as chimeric antigen receptor or an engineered T-cell receptor. For example, treatment can involve the simultaneous administration of the fusion protein with a chimeric receptor. In some embodiments, the treatment can involve the co-administration of a fusion protein and a modified effector cell comprising a chimeric receptor. In some embodiments, the treatment can involve the sequential administration of a modified effector cell which comprises a chimeric receptor followed by the administration of the fusion protein. In another embodiment, the treatment can involve the sequential administration of a fusion protein followed by the administration of a modified effector cell which comprises a chimeric receptor. In one aspect, there can be a lag of at least 0.5, 1, 2, 3, 4, 5, 6, 8, 10, 12, 15, 18, 20, or 24 hours between administrations. In another aspect, there can be at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 75, 90 or more days in between administrations.

In some embodiments, modified immune effector cells are modified immune cells that comprise T cells and/or natural killer cells. T cells or T lymphocytes are a subtype of white blood cells that are involved in cell-mediated immunity. Exemplary T cells include T helper cells, cytotoxic T cells, TH17 cells, stem memory T cells (TSCM), naïve T cells, memory T cells, effector T cells, regulatory T cells, or natural killer T cells. In certain aspects, the embodiments described herein include making and/or expanding the modified immune effector cells (e.g., T-cells, Tregs, NK-cell or NK T-cells) that comprises transfecting the cells with an expression vector containing a DNA (or RNA) construct encoding the chimeric receptor.

In some embodiments, described herein includes a modified effector cell which comprises a chimeric receptor expressed on the surface of the cell. In some instances, the chimeric receptor comprises an antigen binding region that enables recognition and binding to a tumor antigen, e.g., a tumor-associated antigen or a tumor-specific antigen. In some instances, the antigen binding region comprises an antibody or binding fragment, for example, an Fab, an Fab′, an F(ab′)₂, an F(ab′)₃, an scFv, an sc (Fv)₂, a dsFv, a diabody, a minibody, and a nanobody or binding fragments thereof. In some cases, the antigen binding region comprises a scFv. In some cases, the chimeric receptor comprises a scFv (e.g., a chimeric antigen receptor (CAR)). In some instances, the chimeric antigen receptor comprises a pattern-recognition receptor. In other cases, the chimeric receptor comprises an engineered T-cell receptor (TCR).

Further provided herein is an immune effector cell comprising a cell tag for use as a kill switch, selection marker, a biomarker, or a combination thereof. In some embodiments, the cell tag comprises HERIt, HERIt-1, CD20t-1 or CD20. In some cases, the cell tag comprises HER It, and said HER It comprises the polypeptide sequence of SEQ ID NO: 68. In some instances, the cell tag comprises HER It-1, and the HERIt-1 comprises the polypeptide sequence of SEQ ID NO: 69.

### Chimeric Antigen Receptors (CARs)

A chimeric antigen receptor (CAR) is an engineered receptor which grafts an exogenous specificity onto an immune effector cell. In some instances, a CAR comprises an extracellular domain (ectodomain) that comprises an antigen binding domain, a stalk region, a transmembrane domain and an intracellular (endodomain) domain. In some instances, the intracellular domain further comprises one or more intracellular signaling domains. In some instances, a CAR described herein comprises an antigen binding domain, a stalk region, a transmembrane domain, one or more costimulatory domains, and a signaling domain for T-cell activation.

An antigen binding domain can comprise complementary determining regions of a monoclonal antibody, variable regions of a monoclonal antibody, and/or antigen binding fragments thereof. A complementarity determining region (CDR) is a short amino acid sequence found in the variable domains of antigen receptor (e.g., immunoglobulin and T-cell receptor) proteins that complements an antigen and therefore provides the receptor with its specificity for that particular antigen. Each polypeptide chain of an antigen receptor can contain three CDRs (CDRI. CDR2, and CDR3). In some instances, an antigen binding domain comprises F(ab′)₂. Fab′, Fab. Fv, or scFv. In some cases, an antigen binding domain is a scFv. In some cases, an antigen binding domain is a Fab. In some cases, an antigen binding domain is a Fab′. In some cases, an antigen binding domain is F(ab′)₂. In some cases, an antigen binding domain is an Fv.

In some embodiments, a CAR described herein comprises an antigen binding domain that binds to an epitope on CD19, BCMA, CD23, BAFF-R, GPRC5D, CD44, CAIX, CD5, CD30, CD70, CD44v6, CD44v7, CD44v8, CD174, CD28, CD128, CD138, CS1, CLL-1, L1-CAM, FAP, ROR1, CEA, EGP-2, EGP-40, HER2, HER3, Folate-binding Protein, GD2, GD3, IL-13R-a2, IL-11Ra, EphA2, CSPG4, KDR, EDB-F, mesothelin, CD22, EGFR, Folate receptor α, MUC-1, MUC-4, MUC-16, MAGE-A1, h5T4, PSMA, PSCA, GPC3, c-met, TAG-72, EGFR, CD20, EGFRVIII, CD123 or VEGF-R2. In some embodiments, a CAR described herein comprises an antigen binding domain that binds to an epitope on MUC16. In some embodiments, a CAR described herein comprises an antigen binding domain that binds to an epitope on CD19 or CD33. In some instances, a CAR described herein comprises an antigen binding domain that binds to an epitope on CD19. In some cases, a CAR described herein comprises an antigen binding domain that binds to an epitope on CD33. In further embodiments, a CAR described herein comprises an autoantigen or an antigen binding region that binds to an epitope on HLA-A2, myelin oligodendrocyte glycoprotein (MOG), factor VIII (FVIII), MAdCAMI, SDF1, or collagen type II.

In some embodiments, the CARs and methods described herein can be used for the treatment of a hyperproliferative disease, such as a cancer, an autoimmune disease or for the treatment of an infection, such as a viral, bacterial or parasitic infection. In some aspects, the CAR targets an antigen that is elevated in cancer cells, in autoimmune cells or in cells that are infected by a virus, bacteria or parasite. Pathogens that may be targeted include, without limitation, Plasmodium, trypanosome, Aspergillus, Candida, Hepatitis A, Hepatitis B, Hepatitis C, HSV, HPV, RSV, EBV, CMV, JC virus, BK virus, or Ebola pathogens. Autoimmune diseases can include graft-versus-host disease, rheumatoid arthritis, lupus, celiac disease, Crohn's disease, Sjögren Syndrome, polymyalgia rheumatic, multiple sclerosis, neuromyelitis optica, ankylosing spondylitis, Type 1 diabetes, alopecia areata, vasculitis, temporal arteritis, bullous pemphigoid, psoriasis, pemphigus vulgaris, or autoimmune uveitis.

The pathogen recognized by a CAR may be essentially any kind of pathogen, but in some embodiments the pathogen is a fungus, bacteria, or virus. Exemplary viral pathogens include those of the families of Adenoviridae, Epstein-Barr virus (EBV), Cytomegalovirus (CMV), Respiratory Syncytial Virus (RSV), JC virus, BK virus, HPV, HSV, HHV family of viruses, Hepatitis family of viruses, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae. Exemplary pathogenic viruses cause smallpox, influenza, mumps, measles, chickenpox, ebola, and rubella. Exemplary pathogenic fungi include Candida, Aspergillus, Cryptococcus, Histoplasma, Pneumocystis, and Stachybotrys. Exemplary pathogenic bacteria include Streptococcus, Pseudomonas, Shigella, Campylobacter, Staphylococcus, Helicobacter, E. coli, Rickettsia, Bacillus, Bordetella, Chlamydia, Spirochetes, and Salmonella. In some embodiments the pathogen receptor Dectin-1 may be used to generate a CAR that recognizes the carbohydrate structure on the cell wall of fungi such as Aspergillus. In another embodiment, CARs can be made based on an antibody recognizing viral determinants (e.g., the glycoproteins from CMV and Ebola) to interrupt viral infections and pathology.

In some embodiments, a “stalk” region, or a “spacer” or “hinge” region, is used to link the antigen-binding domain to the transmembrane domain. In some instances, a “stalk domain” or “stalk region” comprise any oligonucleotide- or polypeptide that functions to link the transmembrane domain to, either the extracellular domain or, the cytoplasmic domain in the polypeptide chain. In some embodiments, it is flexible enough to allow the antigen-binding domain to orient in different directions to facilitate antigen recognition. In some instances, the stalk region comprises the hinge region from IgG1. In alternative instances, the stalk region comprises the CH2CH3 region of immunoglobulin and optionally portions of CD3. In some cases, the stalk region comprises a CD8a hinge region, an IgG4-Fc 12 amino acid hinge region (ESKYGPPCPPCP) (SEQ ID NO: 561) or IgG4 hinge regions as described in WO/2016/073755.

The transmembrane domain can be derived from either a natural or a synthetic source. Where the source is natural, the domain can be derived from any membrane-bound or transmembrane protein. Suitable transmembrane domains can include the transmembrane region(s) of alpha, beta or zeta chain of the T-cell receptor; or a transmembrane region from CD28, CD3 epsilon, CD35, CD45, CD4, CD5, CD8alpha, CD9, CD16, CD22, CD33, CD37, CD64, CD80, CD86, CD134, CD137 or CD154. Alternatively the transmembrane domain can be synthetic, and can comprise hydrophobic residues such as leucine and valine. In some embodiments, a triplet of phenylalanine, tryptophan and valine is found at one or both termini of a synthetic transmembrane domain. Optionally, a short oligonucleotide or polypeptide linker, in some embodiments, between 2 and 10 amino acids in length may form the linkage between the transmembrane domain and the cytoplasmic signaling domain of a CAR. In some embodiments, the linker is a glycine-serine linker.

In some embodiments, the transmembrane domain comprises a CD8a transmembrane domain or a CD35 transmembrane domain. In some embodiments, the transmembrane domain comprises a CD8a transmembrane domain. In other embodiments, the transmembrane domain comprises a CD35 transmembrane domain.

The intracellular domain can comprise one or more costimulatory domains. Exemplary costimulatory domains include, but are not limited to, CD8, CD27, CD28, 4-1BB (CD137), ICOS, DAP10, DAP12, OX40 (CD 134) or fragment or combination thereof. In some instances, a CAR described herein comprises one or more, or two or more of costimulatory domains selected from CD8, CD27, CD28, 4-1BB (CD137), ICOS, DAP10, DAP12, OX40 (CD134) or fragment or combination thereof. In some instances, a CAR described herein comprises one or more, or two or more of costimulatory domains selected from CD27, CD28, 4-1BB (CD137), ICOS, OX40 (CD 134) or fragment or combination thereof. In some instances, a CAR described herein comprises one or more, or two or more of costimulatory domains selected from CD8, CD28, 4-1BB (CD137), or fragment or combination thereof. In some instances, a CAR described herein comprises one or more, or two or more of costimulatory domains selected from CD28, 4-1BB (CD137), or fragment or combination thereof. In some instances, a CAR described herein comprises costimulatory domains CD28 and 4-1BB (CD137) or their respective fragments thereof. In some instances, a CAR described herein comprises costimulatory domains CD28 and OX40 (CD134) or their respective fragments thereof. In some instances, a CAR described herein comprises costimulatory domains CD8 and CD28 or their respective fragments thereof. In some instances, a CAR described herein comprises costimulatory domains CD28 or a fragment thereof. In some instances, a CAR described herein comprises costimulatory domains 4-1BB (CD137) or a fragment thereof. In some instances, a CAR described herein comprises costimulatory domains OX40 (CD134) or a fragment thereof. In some instances, a CAR described herein comprises costimulatory domains CD8 or a fragment thereof.

In some embodiments, the intracellular domain further comprises a signaling domain for T-cell activation. In some instances, the signaling domain for T-cell activation comprises a domain derived from TCR zeta, FcR gamma, FcR beta, CD3 gamma, CD3 delta, CD3 epsilon, CD5, CD22, CD79a, CD79b or CD66d. In some cases, the signaling domain for T-cell activation comprises a domain derived from CD35.

In some embodiments, a CAR described herein is administered to a subject with one or more additional therapeutic agents that include but are not limited to cytokines as described herein. In further embodiments, an immune effector cell expressing a CAR described herein expresses membrane-bound IL-15 (“mIL-15 or mbIL-15”). In aspects of the invention, the mbIL-15 comprises a fusion protein between IL-15 and IL-15Rα. In further embodiments, the mbIL-15 comprises an amino acid sequence having at least 90%, 91%, 92%, 93%, 94%, 95%, 96%, 97%, 98%, 99% or 100% identity with the amino acid sequence of SEQ ID NO: 69. In certain cases, the CAR and the cytokine is expressed in separate vectors. In specific cases, the vectors can be lentiviral vectors, retroviral vectors or Sleeping Beauty transposons.

### CD19-Specific CARs

CD19 is a cell surface glycoprotein of the immunoglobulin superfamily and is found predominately in malignant B-lineage cells. In some instances, CD19 has also been detected in solid tumors such as pancreatic cancer, liver cancer, and prostate cancer.

In some embodiments, described herein include a CD19-specific CAR, in which the antigen binding domain comprises a F(ab′)₂, Fab′, Fab, Fv, or scFv. In some instances, the antigen binding domain recognizes an epitope on CD19. In some embodiments, the antigen binding domain recognizes an epitope on CD19 that is also recognized by FMC63. In some embodiments the scFv and/or V_{H}/V_{L} domains is derived from FMC63. FMC63 generally refers to a mouse monoclonal IgG1 antibody raised against Nalm-1 and -16 cells expressing CD 19 of human origin (Ling, N. R., el al. (1987). Leucocyte typing III. 302). In some instances, the CD19-specific CAR-T cell further comprises a transmembrane domain selected from a CD8alpha transmembrane domain or a CD35 transmembrane domain; one or more costimulatory domains selected from CD27, CD28, 4-1BB (CD137), ICOS, DAP10, OX40 (CD134) or fragment or combination thereof; and a signaling domain from CD35.

In some embodiments, the antigen binding domain recognizes an epitope on CD19 that is also recognized by JCAR014. JCAR015, JCAR017, or 19-28z CAR (Juno Therapeutics). In some embodiments, described herein include a CD19-specific CAR-T cell, in which the antigen binding domain recognizes an epitope on CD 19 that is also recognized by JCAR014, JCAR015. JCAR017, or 19-282 CAR (Juno Therapeutics). In some instances, the CD19-specific CAR-T cell further comprises a transmembrane domain selected from a CD8alpha transmembrane domain or a CD35 transmembrane domain; one or more costimulatory domains selected from CD27, CD28, 4-1BB (CD137), ICOS, DAP10, OX40 (CD134) or fragment or combination thereof; and a signaling domain from CD3.

In some embodiments, described herein include a CD19-specific CAR-T cell comprises a scFv antigen binding domain, and the antigen binding domain recognizes an epitope on CD19 that is also recognized by JCAR014, JCAR015, JCAR017, or 19-28z CAR (Juno Therapeutics). In some instances, the CD19-specific CAR-T cell further comprises a transmembrane domain selected from a CD8alpha transmembrane domain or a CD35 transmembrane domain; one or more costimulatory domains selected from CD27, CD28, 4-1BB (CD137), ICOS, DAP10, OX40 (CD134) or fragment or combination thereof; and a signaling domain from CD35.

In some embodiments, a CD19-specific CAR-T cell described herein comprises an anti-CD19 antibody described in US20160152723. In some embodiments, a CD19-specific CAR-T cell described herein comprises an anti-CD19 antibody described in WO2015/123642. In some embodiments, a CD19-specific CAR-T cell described herein comprises an anti-CD19 scFv derived from clone FMC63 (Nicholson et al. Construction and characterization of a functional CD19 specific single chain Fv fragment for immunotherapy of B lineage leukemia and lymphoma. Mol. Immunol., 34:1157-1165, 1997).

In some embodiments, the antigen binding domain recognizes an epitope on CD19 that is also recognized by KTE-C19 (Kite Pharma, Inc.). In some embodiments, described herein include a CD19-specific CAR-T cell, in which the antigen binding domain recognizes an epitope on CD 19 that is also recognized by KTE-C19. In some instances, the CD19-specific CAR-T cell further comprises a transmembrane domain selected from a CD8alpha transmembrane domain or a CD32 transmembrane domain; one or more costimulatory domains selected from CD27, CD28, 4-1BB (CD137), ICOS, DAP10, OX40 (CD134) or fragment or combination thereof; and a signaling domain from CD35.

In some embodiments, described herein include a CD19-specific CAR-T cell comprises a scFv antigen binding domain, and the antigen binding domain recognizes an epitope on CD19 that is also recognized by KTE-C19. In some instances, the CD19-specific CAR-T cell further comprises a transmembrane domain selected from a CD8alpha transmembrane domain or a CD32 transmembrane domain; one or more costimulatory domains selected from CD27, CD28, 4-1BB (CD137), ICOS, DAP10, DAP12, OX40 (CD134) or fragment or combination thereof; and a signaling domain from CD35.

In some embodiments, a CD19-specific CAR-T cell described herein comprises an anti-CD19 antibody described in WO2015187528 or fragment or derivative thereof.

In some embodiments, the antigen binding domain recognizes an epitope on CD19 that is also recognized by CTL019 (Novartis). In some embodiments, described herein include a CD19-specific CAR-T cell, in which the antigen binding domain recognizes an epitope on CD19 that is also recognized by CTL019. In some instances, the CD19-specific CAR-T cell further comprises a transmembrane domain selected from a CD8alpha transmembrane domain or a CD35 transmembrane domain; one or more costimulatory domains selected from CD27, CD28, 4-1BB (CD137), ICOS, DAP10, DAP12, OX40 (CD134) or fragment or combination thereof; and a signaling domain from CD35.

In some embodiments, described herein include a CD19-specific CAR-T cell comprises a scFv antigen binding domain, and the antigen binding domain recognizes an epitope on CD19 that is also recognized by CTL019. In some instances, the CD19-specific CAR-T cell further comprises a transmembrane domain selected from a CD8alpha transmembrane domain or a CD3 transmembrane domain; one or more costimulatory domains selected from CD27, CD28, 4-1BB (CD137), ICOS, DAP10, DAP12, OX40 (CD134) or fragment or combination thereof; and a signaling domain from CD35.

In some embodiments, the antigen binding domain recognizes an epitope on CD19 that is also recognized by UCART19 (Cellectis). In some embodiments, described herein include a CD19-specific CAR-T cell, in which the antigen binding domain recognizes an epitope on CD 19 that is also recognized by UCART19. In some instances, the CD19-specific CAR-T cell further comprises a transmembrane domain selected from a CD8alpha transmembrane domain or a CD35 transmembrane domain; one or more costimulatory domains selected from CD27, CD28, 4-1BB (CD137), ICOS, DAP10, DAP12, OX40 (CD134) or fragment or combination thereof; and a signaling domain from CD3.

In some embodiments, described herein include a CD19-specific CAR-T cell comprises a scFv antigen binding domain, and the antigen binding domain recognizes an epitope on CD19 that is also recognized by UCART19. In some instances, the CD19-specific CAR-T cell further comprises a transmembrane domain selected from a CD8alpha transmembrane domain or a CD32 transmembrane domain; one or more costimulatory domains selected from CD27, CD28, 4-1BB (CD137), ICOS, DAP10, DAP12, OX40 (CD134) or fragment or combination thereof; and a signaling domain from CD35.

In some embodiments, the antigen binding domain recognizes an epitope on CD19 that is also recognized by BPX-401 (Bellicum). In some embodiments, described herein include a CD19-specific CAR-T cell, in which the antigen binding domain recognizes an epitope on CD19 that is also recognized by BPX-401. In some instances, the CD19-specific CAR-T cell further comprises a transmembrane domain selected from a CD8alpha transmembrane domain or a CD32 transmembrane domain; one or more costimulatory domains selected from CD27, CD28, 4-1BB (CD137), ICOS, DAP10, DAP12, OX40 (CD134) or fragment or combination thereof; and a signaling domain from CD32.

In some embodiments, described herein include a CD19-specific CAR-T cell comprises a scFv antigen binding domain, and the antigen binding domain recognizes an epitope on CD19 that is also recognized by BPX-401. In some instances, the CD19-specific CAR-T cell further comprises a transmembrane domain selected from a CD8alpha transmembrane domain or a CD3 transmembrane domain; one or more costimulatory domains selected from CD27, CD28, 4-1BB (CD137), ICOS, DAP10, DAP12, OX40 (CD134) or fragment or combination thereof; and a signaling domain from CD35.

In some cases, the antigen binding domain recognizes an epitope on CD 19 that is also recognized by blinatumomab (Amgen), coltuximabravtansine (ImmunoGen Inc./Sanofi-aventis). MOR208 (Morphosys AG/Xencor Inc.), MEDI-551 (Mcdimmune), denintuzumabmafodotin (Seattle Genetics). B4 (or DI-B4) (Merck Serono), taplitumomabpaptox (National Cancer Institute), XmAb 5871 (Amgen/Xencor, Inc.). MDX-1342 (Medarex) or AFM11 (Affimed). In some instances, the CD19-specific CAR further comprises a transmembrane domain selected from a CD8alpha transmembrane domain or a CD3 transmembrane domain; one or more costimulatory domains selected from CD27, CD28, 4-1BB (CD137), ICOS, DAP10, OX40 (CD134) or fragment or combination thereof; and a signaling domain from CD35.

In some embodiments, described herein include a CD19-specific CAR-T cell, in which the antigen binding domain comprises a F(ab′), Fab′, Fab, Fv, or scFv. In some instances, the antigen binding domain recognizes an epitope on CD19. In some cases, the antigen binding domain recognizes an epitope on CD19 that is also recognized by blinatumomab (Amgen), coltuximabravtansine (ImmunoGen Inc./Sanofi-aventis), MOR208 (Morphosys AG/Xencor Inc.). MEDI-551 (Medimmunc), denintuzumabmafodotin (Seattle Genetics), B4 (or DI-B4) (Merck Serono), taplitumomabpaptox (National Cancer Institute), XmAb 5871 (Amgen/Xencor, Inc.), MDX-1342 (Medarex) or AFM11 (Affimed). In some instances, the CD19-specific CAR-T cell further comprises a transmembrane domain selected from a CD8alpha transmembrane domain or a CD32 transmembrane domain; one or more costimulatory domains selected from CD27, CD28, 4-1BB (CD137), ICOS, DAP10, DAP12, OX40 (CD134) or fragment or combination thereof; and a signaling domain from CD35.

In some cases, a CD19-specific CAR-T cell described herein comprise a scFv antigen binding domain, and the antigen binding domain recognizes an epitope on CD19 that is also recognized by FMC63, blinatumomab (Amgen), coltuximabravtansine (ImmunoGen Inc./Sanofi-aventis), MOR208 (Morphosys AG/Xencor Inc.), MEDI-551 (Medimmune), denintuzumabmafodotin (Scattle Genctics). B4 (or DI-B4) (Merck Serono), taplitumomabpaptox (National Cancer Institute), XmAb 5871 (Amgen/Xencor, Inc.), MDX-1342 (Medarex) or AFM11 (Affimed). In some instances, the CD19-specific CAR-T cell further comprises a transmembrane domain selected from a CD8alpha transmembrane domain or a CD35 transmembrane domain; one or more costimulatory domains selected from CD27, CD28, 4-1BB (CD137), ICOS, DAP10, DAP12, OX40 (CD134) or fragment or combination thereof; and a signaling domain from CD3%.

### CD33-Specific CARs

“CD33,” is a 67 kDa single pass transmembrane glycoprotein and is a member of the sialic acid-binding immunoglobulin-like lectins (Siglecs) super-family. CD33 is characterized by a V-set Ig-like domain responsible for sialic acid binding and a C2-set Ig-like domain in its extracellular domain. Alternative splicing of CD33 mRNA leads to a shorter isoform (CD33m) lacking the V-set Ig-like domain as well as the disulfide bond linking the V- and C2-set Ig-like domains. In healthy subjects, CD33 is primarily expressed as a myeloid differentiation antigen found on normal multipotent myeloid precursors, unipotent colony-forming cells, monocytes and maturing granulocytes. CD33 is expressed on more than 80% of myeloid leukemia cells but not on normal hematopoietic stem cells or mature granulocytes. (Andrews, R. et al., The L4F3 antigen is expressed by unipotent and multipotent colony-forming cells but not by their precursors, Blood, 68 (5): 1030-5 (1986)). CD33 has been reported to be expressed on malignant myeloid cells, activated T cells and activated NK cells and is found on at least a subset of blasts in the vast majority of AML patients (Pollard, J. et al., Correlation of CD33 expression level with disease characteristics and response to gemtuzumab ozogamicin containing chemotherapy in childhood AML, Blood, 119 (16): 3705-11 (2012)). In addition to broad expression on AML blasts, CD33 may be expressed on stem cells underlying AML.

In embodiments, the antigen binding moiety of a CAR described herein is specific to CD33 (CD33 CAR). The CD33-specific CAR, when expressed on the cell surface, redirects the specificity of T cells to human CD33. In embodiments, the antigen binding domain comprises a single chain antibody fragment (scFv) comprising a variable domain light chain (V_{L}) and variable domain heavy chain (VH) of a target antigen specific monoclonal anti-CD33 antibody joined by a flexible linker, such as a glycine-serine linker or a Whitlow linker. In embodiments, the scFv are M195, m2H12, DRB2, and/or My9-6. In embodiments, the scFv is humanized, for example, hM195. In some embodiments, the antigen binding moiety may comprise VH and VL that are directionally linked, for example, from N to C terminus, VH-linker-VL or VL-linker-VH.

In embodiments, a CAR described herein comprises an antigen-binding moiety comprising a VL polypeptide having at least 90%, 91%, 92%, 93%, 94%, 95%, 96%, 97%, 98%, 99% or 100% identity with the amino acid sequence of SEQ ID NO:35 (hM195 VL).

In embodiments, a CAR described herein comprises an antigen-binding moiety comprising a VH polypeptide having at least 90%, 91%, 92%, 93%, 94%, 95%, 96%, 97%, 98%, 99% or 100% identity with the amino acid sequence of SEQ ID NO:36 (hM195 VH).

In embodiments, a CAR described herein comprises an antigen-binding moiety comprising a VH polypeptide having at least 90%, 91%, 92%, 93%, 94%, 95%, 96%, 97%, 98%, 99% or 100% identity with the amino acid sequence of SEQ ID NO:37 (M2H12 VH).

In embodiments, a CAR described herein comprises an antigen-binding moiety comprising a VL polypeptide having at least 90%, 91%, 92%, 93%, 94%, 95%, 96%, 97%, 98%, 99% or 100% identity with the amino acid sequence of SEQ ID NO:38 (M2H12 VL).

In embodiments, a CAR described herein comprises an antigen-binding moiety comprising a VH polypeptide having at least 90%, 91%, 92%, 93%, 94%, 95%, 96%, 97%, 98%, 99% or 100% identity with the amino acid sequence of SEQ ID NO:39 (DRB2 VH).

In embodiments, a CAR described herein comprises an antigen-binding moiety comprising a VL polypeptide having at least 90%, 91%, 92%, 93%, 94%, 95%, 96%, 97%, 98%, 99% or 100% identity with the amino acid sequence of SEQ ID NO:40 (DRB2 VL).

In embodiments, a CAR described herein comprises an antigen-binding moiety comprising a VH polypeptide having at least 90%, 91%, 92%, 93%, 94%, 95%, 96%, 97%, 98%, 99% or 100% identity with the amino acid sequence of SEQ ID NO:41 (My9-6 VH).

In embodiments, a CAR described herein comprises an antigen-binding moiety comprising a VL polypeptide having at least 90%, 91%, 92%, 93%, 94%, 95%, 96%, 97%, 98%, 99% or 100% identity with the amino acid sequence of SEQ ID NO:42 (My9-6 VL).

### MUC16-Specific CARs

MUC16 is a large carbohydrate antigen, also known as CA-125. MUC16 is encoded by the MUC16 gene located on human chromosome 19. MUC16 is a highly glycosylated multidomain type I transmembrane protein comprising 3 domains. The C-terminal domain contains multiple extracellular SEA (sea urchin sperm protein, enterokinase, and agrin) modules that have an autoproteolytic activity. SEA harbors two proteolytic sites proximal to the transmembrane (TM) domain. A large cleaved domain termed CA-125 is released into circulation at acidic pH. CA-125 is commonly used as disease biomarker for ovarian cancer. The highly conserved truncated extracellular membrane tethered protein domain called MUC16ecto domain. A MUC16 antibody was identified that specifically bound the ectodomain of MUC16 that is retained on the tumor cell surface. “Overexpression of MUC16” by a cell of interest (such as a cancer cell) refers to a higher level of MUC16 protein and/or mRNA that is expressed by the cell of interest compared to a control cell (such as a non-cancerous cell, normal cell, etc.).

In embodiments, the antigen binding moiety of a CAR described herein is specific to MUC16 (MUC16 CAR). The MUC16-specific CAR, when expressed on the cell surface, redirects the specificity of T cells to human MUC16. In embodiments, the antigen binding domain comprises a single chain antibody fragment (scFv) comprising a variable domain light chain (VL) and variable domain heavy chain (VH) of a target antigen specific monoclonal anti-MUC16 antibody joined by a flexible linker, such as a glycine-serine linker or a Whitlow linker. In embodiments, the scFv are MUC16-1 scFv (SEQ ID NOs: 43-44), MUC16-2 scFv (SEQ ID NOs: 45-46), MUC16-3 scFv (SEQ ID NOs: 47-48), MUC16-4 scFv (SEQ ID NOS: 49-50), MUC16-5 scFv (SEQ ID NOs: 51-52), MUC16-6 scFv (SEQ ID NOs: 53-54) or MUC16-7 scFv (SEQ ID NOs: 55-56). In embodiments, the scFv is humanized.

In embodiments, a CAR described herein comprises an antigen-binding moiety comprising a VL polypeptide having at least 90%, 91%, 92%, 93%, 94%, 95%, 96%, 97%, 98%, 99% or 100% identity with the amino acid sequence of SEQ ID NO:43 (MUC16-1).

In embodiments, a CAR described herein comprises an antigen-binding moiety comprising a VH polypeptide having at least 90%, 91%, 92%, 93%, 94%, 95%, 96%, 97%, 98%, 99% or 100% identity with the amino acid sequence of SEQ ID NO:44 (MUC16-1).

In embodiments, a CAR described herein comprises an antigen-binding moiety comprising a VL polypeptide having at least 90%, 91%, 92%, 93%, 94%, 95%, 96%, 97%, 98%, 99% or 100% identity with the amino acid sequence of SEQ ID NO:45 (MUC16-2).

In embodiments, a CAR described herein comprises an antigen-binding moiety comprising a VH polypeptide having at least 90%, 91%, 92%, 93%, 94%, 95%, 96%, 97%, 98%, 99% or 100% identity with the amino acid sequence of SEQ ID NO:46 (MUC16-2).

In embodiments, a CAR described herein comprises an antigen-binding moiety comprising a VL polypeptide having at least 90%, 91%, 92%, 93%, 94%, 95%, 96%, 97%, 98%, 99% or 100% identity with the amino acid sequence of SEQ ID NO:47 (MUC16-3).

In embodiments, a CAR described herein comprises an antigen-binding moiety comprising a VH polypeptide having at least 90%, 91%, 92%, 93%, 94%, 95%, 96%, 97%, 98%, 99% or 100% identity with the amino acid sequence of SEQ ID NO:48 (MUC16-3).

In embodiments, a CAR described herein comprises an antigen-binding moiety comprising a VL polypeptide having at least 90%, 91%, 92%, 93%, 94%, 95%, 96%, 97%, 98%, 99% or 100% identity with the amino acid sequence of SEQ ID NO:49 (MUC16-4).

In embodiments, a CAR described herein comprises an antigen-binding moiety comprising a VH polypeptide having at least 90%, 91%, 92%, 93%, 94%, 95%, 96%, 97%, 98%, 99% or 100% identity with the amino acid sequence of SEQ ID NO:50 (MUC16-4).

In embodiments, a CAR described herein comprises an antigen-binding moiety comprising a VL polypeptide having at least 90%, 91%, 92%, 93%, 94%, 95%, 96%, 97%, 98%, 99% or 100% identity with the amino acid sequence of SEQ ID NO:51 (MUC16-5).

In embodiments, a CAR described herein comprises an antigen-binding moiety comprising a VL polypeptide having at least 90%, 91%, 92%, 93%, 94%, 95%, 96%, 97%, 98%, 99% or 100% identity with the amino acid sequence of SEQ ID NO:52 (MUC16-5).

In embodiments, a CAR described herein comprises an antigen-binding moiety comprising a VH polypeptide having at least 90%, 91%, 92%, 93%, 94%, 95%, 96%, 97%, 98%, 99% or 100% identity with the amino acid sequence of SEQ ID NO:53 (MUC16-5).

In embodiments, a CAR described herein comprises an antigen-binding moiety comprising a VL polypeptide having at least 90%, 91%, 92%, 93%, 94%, 95%, 96%, 97%, 98%, 99% or 100% identity with the amino acid sequence of SEQ ID NO:54 (MUC16-6).

In embodiments, a CAR described herein comprises an antigen-binding moiety comprising a VL polypeptide having at least 90%, 91%, 92%, 93%, 94%, 95%, 96%, 97%, 98%, 99% or 100% identity with the amino acid sequence of SEQ ID NO:55 (MUC16-7).

In embodiments, a CAR described herein comprises an antigen-binding moiety comprising a VH polypeptide having at least 90%, 91%, 92%, 93%, 94%, 95%, 96%, 97%, 98%, 99% or 100% identity with the amino acid sequence of SEQ ID NO:56 (MUC16-7).

In some embodiments, the antigen binding moiety can comprise VH and VL that are directionally linked, for example, from N to C terminus, VH-linker-VL or VL-linker-VH.

### Engineered T-Cell Receptors (“TCRs”)

In some embodiments, the chimeric receptor comprises an engineered T-cell receptor. The T cell receptor (TCR) is composed of two chains (αβ or γδ) that pair on the surface of the T cell to form a heterodimeric receptor. In some instances, the αβ TCR is expressed on most T cells in the body and is known to be involved in the recognition of specific MHC-restricted antigens. Each a and B chain are composed of two domains: a constant domain (C) which anchors the protein to the cell membrane and is associated with invariant subunits of the CD3 signaling apparatus; and a variable domain (V) that confers antigen recognition through six loops, referred to as complementarity determining regions (CDRs). In some instances, each of the V domains comprises three CDRs; e.g., CDRI, CDR2 and CDR3 with CDR3 as the hypervariable region. These CDRs interact with a complex formed between an antigenic peptide bound to a protein encoded by the major histocompatibility complex (pepMHC) (e.g., HLA-A, HLA-B, HLA-C, HLA-DPAI, HLA-DPBI, HLA-DQAI, HLA-DQBI, HLA-DRA, or HLA-DRB 1 complex). In some instances, the constant domain further comprises a joining region that connects the constant domain to the variable domain. In some cases, the beta chain further comprises a short diversity region which makes up part of the joining region.

In some cases, such TCR are reactive to specific tumor antigen, e.g. NY-ESO, Mage A3, Titin. In other cases, such TCR are reactive to specific neoantigens expressed within a patient's tumor (i.e. patient-specific, somatic, non-synonymous mutations expressed by tumors). In some cases, engineered TCRs can be affinity-enhanced.

In some embodiments, a TCR is described using the International Immunogenetics (IMGT) TCR nomenclature, and links to the IMGT public database of TCR sequences. For example, there can be several types of alpha chain variable (Va) regions and several types of beta chain variable (VB) regions distinguished by their framework, CDR1, CDR2, and CDR3 sequences. As such, a Va type can be referred to in IMGT nomenclature by a unique TRAV number. For example, “TRAV21” defines a TCR Va region having unique framework and CDR1 and CDR2 sequences, and a CDR3 sequence which is partly defined by an amino acid sequence which is preserved from TCR to TCR but which also includes an amino acid sequence which varies from TCR to TCR. Similarly, “TRBV5-1” defines a TCR VB region having unique framework and CDRI and CDR2 sequences, but with only a partly defined CDR3 sequence.

In some cases, the beta chain diversity region is referred to in IMGT nomenclature by the abbreviation TRBD.

In some instances, the unique sequences defined by the IMGT nomenclature are widely known and accessible to those working in the TCR field. For example, they can be found in the IMGT public database and in “T cell Receptor Factsbook,” (2001) LeFranc and LeFranc, Academic Press, ISBN 0-12-441352-8.

In some embodiments, an αβ heterodimeric TCR is, for example, transfected as full length chains having both cytoplasmic and transmembrane domains. In some cases, the TCRs contain an introduced disulfide bond between residues of the respective constant domains, as described, for example, in WO 2006/000830.

In some instances, TCRs described herein are in single chain format, for example see WO 2004/033685. Single chain formats include αβ TCR polypeptides of the Vα-L-Vβ, Vβ-L-Vα, Vα-Cα-L-Vβ, Vα-L-Vβ-Cβ, Vα-Cα-L-Vβ-Cβ types, wherein Va and VB are TCR α and B variable regions respectively, Cα and Cβ are TCR α and β constant regions respectively, and L is a linker sequence. In certain embodiments single chain TCRs of the invention may have an introduced disulfide bond between residues of the respective constant domains, as described in WO 2004/033685.

The TCR described herein may be associated with a detectable label, a therapeutic agent or a PK modifying moiety.

Exemplary detectable labels for diagnostic purposes include, but are not limited to, fluorescent labels, radiolabels, enzymes, nucleic acid probes and contrast reagents.

Therapeutic agents which may be associated with the TCRs described herein include immunomodulators, radioactive compounds, enzymes (perforin for example) or chemotherapeutic agents. To ensure that toxic effects are exercised in the desired location the toxin could be inside a liposome linked to a TCR so that the compound is released in a controlled manner. In some cases, the controlled release minimize damaging effects during the transport in the body and ensure that the toxin has maximum effect after binding of the TCR to the relevant antigen presenting cells.

In some embodiments, additional suitable therapeutic agents include for instance:
a. small molecule cytotoxic agents, e.g., compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cisplatin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate glucuronate, auristatin E vincristine and doxorubicin;
b. peptide cytotoxins, i.e. proteins or fragments thereof with the ability to kill mammalian cells. For example, ricin, diphtheria toxin, pseudomonas bacterial exotoxin A, DNase and RNase;
c. radio-nuclides, i.e. unstable isotopes of elements which decay with the concurrent emission of one or more of α or β particles, or γ rays. For example, iodine 131, rhenium 186, indium 111, yttrium 90, bismuth 210 and 213, actinium 225 and astatine 213; chelating agents may be used to facilitate the association of these radio-nuclides to the high affinity TCRs, or multimers thereof;
d. immuno-stimulants, i.e. immune effector molecules which stimulate immune response. For example, cytokines such as IL-2 and IFN-γ,
e. superantigens and mutants thereof;
f. TCR-HLA fusions;
g. chemokines such as IL-8, platelet factor 4, melanoma growth stimulatory protein, etc;
h. antibodies or fragments thereof, including anti-T cell or NK cell determinant antibodies (e.g. anti-CD3, anti-CD28 or anti-CD16);
i. alternative protein scaffolds with antibody like binding characteristics
j. complement activators; and
k. xenogeneic protein domains, allogeneic protein domains, viral/bacterial protein domains, viral/bacterial peptides.

### Doses

Suitable dosages of the fusion protein and modified immune effector cells used will depend on the age and weight of the subject and the particular drug used. Dosages and therapeutic regimens of the fusion protein and modified immune effector cells can be determined by a skilled artisan.

In certain embodiments, the fusion protein is administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg. In some embodiments, the fusion protein is administered at a dose of about 1 mg/kg, about 3 mg/kg, about 5 mg/kg, about 10 mg/kg, about 20 mg/kg, about 30 mg/kg, or about 40 mg/kg. In some embodiments, the fusion protein is administered at a dose of about 1-3 mg/kg, or about 3-10 mg/kg. In some embodiments, the fusion protein is administered at a dose of about 0.5-2, 2-4, 2-5, 5-15, or 5-20 mg/kg. The dosing schedule can vary from e.g., once a week to once every 2, 3, or 4 weeks. In some embodiments, the fusion protein is administered at a dose from about 10 to 20 mg/kg every other week. In another embodiment, the fusion protein is administered at a dose of about 1 mg/kg once every two weeks, about 3 mg/kg once every two weeks, 10 mg/kg once every two weeks, 3 mg/kg once every four weeks, or 5 mg/kg once every four weeks.

In other embodiments, the fusion protein is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 300 mg or about 400 mg. In some embodiments, the fusion protein is administered at a dose of about 200 mg, about 250 mg, about 300 mg, about 350 mg, about 400 mg, about 450 mg, or about 500 mg. In some embodiments, the fusion protein is administered at a dose of 200 or 300 mg. In some embodiments, the fusion protein is administered at a dose of about 250-450 mg, or about 300-400 mg. In some embodiments, the fusion protein is administered at a dose of about 200-300 mg, 250-350 mg, 300-400 mg, 350-450 mg, or 400-500 mg. The dosing schedule can vary from e.g., once a week to once every 2, 3, 4, 5, or 6 weeks. In one embodiment the fusion protein is administered at a dose from about 300 mg to 400 mg once every three or once every four weeks. In some embodiments, the fusion protein is administered at a dose from about 300 mg once every three weeks. In some embodiments, the fusion protein is administered at a dose from about 400 mg once every four weeks. In some embodiments, the fusion protein is administered at a dose from about 300 mg once every four weeks. In some embodiments, the fusion protein is administered at a dose from about 400 mg once every three weeks. The fusion protein can be administered one or more times, e.g., one, two, three, four, five, six, seven or more times. In some embodiments, the fusion protein is administered six times. The fusion protein can be administered at least 5 days, e.g., about 5, 6, 7, 8, 9, 10, 11, 12, 13, 14, 20, 25, 30, 35, or 40 days, after administration of CAR-expressing cells, e.g., MUC16, CD33, CD19 or BCMA-specific CAR expressing cells. In some embodiments, the fusion protein can be administered about 8 days or about 15 days after administration of CAR-expressing cells, e.g., MUC16 specific CAR expressing cells or CD33 specific CAR expressing cells.

The fusion protein can be administered by a variety of methods known in the art, although for many therapeutic applications, the preferred route/mode of administration is intravenous injection or infusion. For example, the fusion protein can be administered by intravenous infusion at a rate of more than 20 mg/min, e.g., 20-40 mg/min, and typically greater than or equal to 40 mg/min to reach a dose of about 35 to 440 mg/m2, typically about 70 to 310 mg/m2, and more typically, about 110 to 130 mg/m2. In embodiments, the fusion protein can be administered by intravenous infusion at a rate of less than 10 mg/min; preferably less than or equal to 5 mg/min to reach a dose of about 1 to 100 mg/m2, preferably about 5 to 50 mg/m2, about 7 to 25 mg/m2 and more preferably, about 10 mg/m2.

The fusion protein can be administered by intravenous infusion at a rate of more than 20 mg/min, e.g., 20-40 mg/min, and typically greater than or equal to 40 mg/min to reach a dose of about 35 to 440 mg/m2, typically about 70 to 310 mg/m2, and more typically, about 110 to 130 mg/m2. In embodiments, the infusion rate of about 110 to 130 mg/m2 achieves a level of about 3 mg/kg. In other embodiments, the fusion protein can be administered by intravenous infusion at a rate of less than 10 mg/min, e.g., less than or equal to 5 mg/min to reach a dose of about 1 to 100 mg/m2, e.g., about 5 to 50 mg/m2, about 7 to 25 mg/m2, or, about 10 mg/m2. In some embodiments, the antibody is infused over a period of about 30 min.

### Modified Effector Cell Doses

In some embodiments, an amount of modified effector cells is administered to a subject in need thereof and the amount is determined based on the efficacy and the potential of inducing a cytokine-associated toxicity. In some cases, an amount of modified effector cells comprises about 10⁵ to about 10⁹ modified effector cells/kg. In some cases, an amount of modified effector cells comprises about 10⁵ to about 10⁸ modified effector cells/kg. In some cases, an amount of modified effector cells comprises about 10⁵ to about 10⁷ modified effector cells/kg. In some cases, an amount of modified effector cells comprises about 10⁶ to about 10⁹ modified effector cells/kg. In some cases, an amount of modified effector cells comprises about 10⁶ to about 10⁸ modified effector cells/kg. In some cases, an amount of modified effector cells comprises about 10⁷ to about 10⁹ modified effector cells/kg. In some cases, an amount of modified effector cells comprises about 10⁵ to about 10⁶ modified effector cells/kg. In some cases, an amount of modified effector cells comprises about 10⁶ to about 10⁷ modified effector cells/kg. In some cases, an amount of modified effector cells comprises about 10⁷ to about 10⁸ modified effector cells/kg. In some cases, an amount of modified effector cells comprises about 10⁸ to about 10⁹ modified effector cells/kg. In some instances, an amount of modified effector cells comprises about 10⁹ modified effector cells/kg. In some instances, an amount of modified effector cells comprises about 10⁸ modified effector cells/kg. In some instances, an amount of modified effector cells comprises about 10⁷ modified effector cells/kg. In some instances, an amount of modified effector cells comprises about 10⁶ modified effector cells/kg. In some instances, an amount of modified effector cells comprises about 10⁵ modified effector cells/kg.

In some embodiments, the modified effector cells are modified T cells. In some instances, the modified T cells are CAR-T cells. In some cases, an amount of CAR-T cells comprises about 10⁵ to about 10⁹ CAR-T cells/kg. In some cases, an amount of CAR-T cells comprises about 10⁵ to about 10⁸ CAR-T cells/kg. In some cases, an amount of CAR-T cells comprises about 10⁵ to about 10⁷ CAR-T cells/kg. In some cases, an amount of CAR-T cells comprises about 10⁶ to about 10⁹ CAR-T cells/kg. In some cases, an amount of CAR-T cells comprises about 10⁶ to about 10⁸ CAR-T cells/kg. In some cases, an amount of CAR-T cells comprises about 10⁷ to about 10⁹ CAR-T cells/kg. In some cases, an amount of CAR-T cells comprises about 10⁵ to about 10⁶ CAR-T cells/kg. In some cases, an amount of CAR-T cells comprises about 10⁶ to about 10⁷ CAR-T cells/kg. In some cases, an amount of CAR-T cells comprises about 10⁷ to about 10⁸ CAR-T cells/kg. In some cases, an amount of CAR-T cells comprises about 10⁸ to about 10⁹ CAR-T cells/kg. In some instances, an amount of CAR-T cells comprises about 10⁹ CAR-T cells/kg. In some instances, an amount of CAR-T cells comprises about 10⁸ CAR-T cells/kg. In some instances, an amount of CAR-T cells comprises about 10⁷ CAR-T cells/kg. In some instances, an amount of CAR-T cells comprises about 10⁶ CAR-T cells/kg. In some instances, an amount of CAR-T cells comprises about 10⁵ CAR-T cells/kg.

In some embodiments, the CAR-T cells are CD19-specific CAR-T cells. In some cases, an amount of CD19-specific CAR-T cells comprises about 105 to about 10⁹ CAR-T cells/kg. In some cases, an amount of CD19-specific CAR-T cells comprises about 10⁵ to about 10⁸ CAR-T cells/kg. In some cases, an amount of CD19-specific CAR-T cells comprises about 10⁵ to about 10⁷ CAR-T cells/kg. In some cases, an amount of CD19-specific CAR-T cells comprises about 10⁶ to about 10⁹ CAR-T cells/kg. In some cases, an amount of CD19-specific CAR-T cells comprises about 10⁶ to about 10⁸ CAR-T cells/kg. In some cases, an amount of CD19-specific CAR-T cells comprises about 10⁷ to about 10⁹ CAR-T cells/kg. In some cases, an amount of CD19-specific CAR-T cells comprises about 10⁵ to about 10⁶ CAR-T cells/kg. In some cases, an amount of CD19-specific CAR-T cells comprises about 10⁶ to about 10⁷ CAR-T cells/kg. In some cases, an amount of CD19-specific CAR-T cells comprises about 107 to about 10⁸ CAR-T cells/kg. In some cases, an amount of CD19-specific CAR-T cells comprises about 10⁸ to about 10⁹ CAR-T cells/kg. In some instances, an amount of CD19-specific CAR-T cells comprises about 10⁹ CAR-T cells/kg. In some instances, an amount of CD19-specific CAR-T cells comprises about 10⁸ CAR-T cells/kg. In some instances, an amount of CD19-specific CAR-T cells comprises about 10⁷ CAR-T cells/kg. In some instances, an amount of CD19-specific CAR-T cells comprises about 10⁶ CAR-T cells/kg. In some instances, an amount of CD19-specific CAR-T cells comprises about 10⁵ CAR-T cells/kg.

In some embodiments, the modified T cells are engineered TCR T-cells. In some cases, an amount of engineered TCR T-cells comprises about 10⁵ to about 10⁹ TCR cells/kg. In some cases, an amount of engineered TCR cells comprises about 10⁵ to about 10⁸ TCR cells/kg. In some cases, an amount of engineered TCR cells comprises about 10⁵ to about 10⁷ TCR cells/kg. In some cases, an amount of engineered TCR cells comprises about 10⁶ to about 10⁹ TCR cells/kg. In some cases, an amount of engineered TCR cells comprises about 10⁶ to about 10⁸ TCR cells/kg. In some cases, an amount of engineered TCR cells comprises about 10⁷ to about 10⁹ TCR cells/kg. In some cases, an amount of engineered TCR cells comprises about 10⁵ to about 10⁶ TCR cells/kg. In some cases, an amount of engineered TCR cells comprises about 10⁶ to about 10⁷ TCR cells/kg. In some cases, an amount of engineered TCR cells comprises about 10⁷ to about 10⁸ TCR cells/kg. In some cases, an amount of engineered TCR cells comprises about 10⁸ to about 10⁹ TCR cells/kg. In some instances, an amount of engineered TCR cells comprises about 10⁹ TCR cells/kg. In some instances, an amount of engineered TCR cells comprises about 10⁸ TCR cells/kg. In some instances, an amount of engineered TCR cells comprises about 10⁷ TCR cells/kg. In some instances, an amount of engineered TCR cells comprises about 10⁶ TCR cells/kg. In some instances, an amount of engineered TCR cells comprises about 10⁵ TCR cells/kg.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### Expression and Purification System of Fusion Proteins

The fusion protein or a fragment or a variant thereof described herein can be produced from cells by culturing a host cell transformed with the expression vector comprising one or more polynucleotides encoding the fusion protein or a fragment or a variant thereof, under conditions, and for an amount of time, sufficient to allow expression of the fusion protein, or a fragment or a variant thereof. For example, polypeptides expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning-A Laboratory Manual-3rd Ed., Cold Spring Harbor Laboratory Press, New York (2001)). The choice of codons, suitable expression vectors and suitable host cells can vary depending on a number of factors, and can be easily optimized as needed. The fusion protein or a fragment or a variant thereof described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the fusion protein or a fragment or a variant thereof can be purified or isolated. The term “purified” or “isolated” as applied to any of the fusion proteins or fragments or variants thereof described herein can refer to a polypeptide or a protein that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9% by weight of the total protein in a sample.

The fusion protein or a fragment or a variant thereof described herein can be isolated or purified in a variety of ways depending on what other components are present in the sample. Standard purification methods include electrophoretic, molecular, immunological, and chromatographic techniques, including ion exchange, hydrophobic, affinity, and reverse-phase HPLC chromatography. For example, the fusion protein or a fragment or a variant thereof can be purified using a standard anti-fusion protein antibody affinity column. Ultrafiltration and diafiltration techniques, in conjunction with protein concentration, are also useful. (See, e.g., Scopes (1994) “Protein Purification, 3rd edition,” Springer-Verlag, New York City, N.Y.) The degree of purification necessary can vary depending on the desired use. In some instances, no purification of the expressed fusion protein or a fragment or variant thereof is necessary.

Methods for determining the yield or purity of a purified fusion protein or a fragment or a variant thereof can include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain). Once expressed, purified, or after being purified following expression, a fusion protein or a fragment or a variant thereof described herein can be assayed for any one of a numbered of desired properties using in vitro or in vivo assays such as any of those described herein. For example, a fusion protein or a fragment or a variant thereof described herein can be assayed for e.g., its ability to inhibit PD-1 and trap TGF-β.

The fusion protein or a fragment or a variant thereof described herein can be produced using a variety of techniques. For example, a polynucleotide encoding a fusion protein or a fragment or a variant thereof described herein can be inserted into an expression vector that contains transcriptional and translational regulatory sequences, which can include, e.g., promoter sequences, ribosomal binding sites, transcriptional start and stop sequences, translational start and stop sequences, transcription terminator signals, polyadenylation signals, and enhancer or activator sequences. In some embodiments, the regulatory sequences can include a promoter and transcriptional start and stop sequences. In addition, the expression vector can include more than one replication system such that it can be maintained in two different organisms, for example, in mammalian or insect cells for expression and in a prokaryotic host for cloning and amplification.

Several possible vector systems are available for the expression of the fusion protein or a fragment or a variant thereof from nucleic acids in mammalian cells (e.g., host cells). For example, one class of vectors relies upon the integration of the desired gene sequences into the host cell genome. Cells which have stably integrated DNA can be selected by simultaneously introducing drug resistance genes such as E. coli gpt (Mulligan and Berg, Proc Natl Acad Sci USA 78:2072 (1981)) or Tn5 neo (Southern and Berg (1982) Mol Appl Genet 1:327). The selectable marker gene can be either linked to the DNA gene sequences to be expressed, or introduced into the same cell by co-transfection (Wigler et al., Cell 16:77 (1979)). Another class of vectors utilizes DNA elements which confer autonomously replicating capabilities to an extrachromosomal plasmid. These vectors can be derived from animal viruses, such as bovine papillomavirus (Sarver et al., Proc Natl Acad Sci USA, 79:7147 (1982)), polyoma virus (Deans et al., Proc Natl Acad Sci USA 81:1292 (1984)), or SV40 virus (Lusky and Botchan, Nature 293:79 (1981)). The expression vectors can be introduced into cells (e.g., host cells) in a manner suitable for subsequent expression of the nucleic acid. The method of introduction is largely dictated by the targeted cell type, discussed below. Non-limiting exemplary methods can include CaPO₄ precipitation, liposome fusion, lipofectin, electroporation, viral infection, dextran-mediated transfection, polybrene-mediated transfection, protoplast fusion, and direct microinjection.

Appropriate host cells for the expression of the fusion protein or a fragment or a variant thereof can include, but not limited to, yeast, bacteria, insect, plant, and, as described above, mammalian cells. Of interest are bacteria such as E. coli, fungi such as Saccharomyces cerevisiae and Pichia pastoris, insect cells such as SF9, mammalian cell lines (e.g., human cell lines), as well as primary cell lines (e.g., primary mammalian cells). In some embodiments, the fusion protein or a fragment or a variant thereof can be expressed in Chinese hamster ovary (CHO) cells or in a suitable myeloma cell line such as (NSO). Suitable cell lines also include, for example, BHK-21 (baby hamster kidney) cells; 293 (human embryonic kidney) cells; HMEpC (Human Mammary Epithelial cells; 3T3 (mouse embryonic fibroblast) cells.

The methods described herein provide for the expression and purification of the fusion protein or a fragment or a variant thereof in various cell-based expression systems, such as protein production in bacterial, mammalian, insect, yeast, and chymadomonas cells. Protein expression can be constitutive or inducible with inducers such as copper sulfate, sugars such as galactose, methanol, methylamine, thiamine, tetracycline, or IPTG. After the fusion protein or a fragment or a variant thereof is expressed in the host cells, the host cells are lysed to liberate the fusion protein or a fragment or a variant thereof for purification. Methods of lysing the various host cells are featured in “Sample Preparation-Tools for Protein Research” EMD Bioscience and in the Current Protocols in Protein Sciences (CPPS). A non-limiting exemplary purification method is affinity chromatography, such as ion-metal affinity chromatograph using nickel, cobalt, or zinc affinity resins for e.g., a histidine-tagged fusion protein or fragment or variant thereof. Methods of purifying histidine-tagged proteins are described by Clontech using their Talonx cobalt resin and by Novagen in their pET system manual, 10th edition. Another non-limiting exemplary purification strategy is by immuno-affinity chromatography, for example, anti-myc antibody conjugated resin can be used to affinity purify e.g., a myc-tagged fusion protein or fragment or variant thereof. Enzymatic digestion with serine proteases, such as thrombin and enterokinase, cleave and release the fusion protein or a fragment or a variant thereof from the histidine or myc tag, releasing the fusion protein or a fragment or a variant thereof from the affinity resin while the histidine-tags and myc-tags are left attached to the affinity resin.

Methods of introducing and expressing polynucleotides encoding the fusion protein or a fragment or a variant thereof into a cell (e.g., a host cell) are known in the art. In the context of an expression vector, the vector comprising the polynucleotide encoding the fusion protein or a fragment or a variant thereof can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. (See, for example, Sambrook et al. (Molecular Cloning: A Laboratory Manual, Cold Spring Harbor Laboratory, New York (2001)). In embodiments, a method for the introduction of a polynucleotide into a host cell is calcium phosphate transfection or polyethylenimine (PEI) Transfection.

Biological methods for introducing polynucleotides encoding the fusion protein or a fragment or a variant thereof into a host cell can include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. Sec, for example, U.S. Pat. Nos. 5,350,674 and 5,585,362.

In some embodiments, the expression vector can have the necessary 5′ upstream and 3′ downstream regulatory elements such as promoter sequences, ribosome recognition and binding TATA box, and 3′ UTR AAUAAA transcription termination sequence for the efficient gene transcription and translation in its respective host cell. The expression vector can have additional sequence such as 6X-histidine (SEQ ID NO: 562), V5, thioredoxin, glutathione-S-transferase, c-Myc, VSV-G, HSV, FLAG, maltose binding peptide, metal-binding peptide, HA and “secretion” signals (Honeybee melittin,.alpha.-factor, PHO, Bip), which are incorporated into the expressed fusion protein or fragment or variant thereof. In addition, there can be enzyme digestion sites incorporated after these sequences to facilitate enzymatic removal of them after they are not needed. These additional sequences are useful for the detection of the fusion protein expression, for protein purification by affinity chromatography, enhanced solubility of the recombinant protein in the host cytoplasm, and/or for secreting the fusion protein out into the culture media, into the periplasm of the prokaryote bacteria, or the spheroplast of the yeast cells. The expression of the fusion protein can be constitutive in the host cells or it can be induced, e.g., with copper sulfate, sugars, such as galactose, methanol, methylamine, thiamine, tetracycline, infection with baculovirus, and (isopropyl-beta-D-thiogalactopyranoside) IPTG, a stable synthetic analog of lactose.

Non-limiting examples of expression vectors and host cells can include the pET vectors (Novagen), pGEX vectors (Amersham Pharmacia), and pMAL vectors (New England labs. Inc.) for protein expression in E. coli host cell such as BL21, BL21 (DE3) and AD494 (DE3) pLysS, Rosetta (DE3), and Origami (DE3) (Novagen); the strong CMV promoter-based pcDNA3.1 (Invitrogen) and pCIneo vectors (Promega) for expression in mammalian cell lines such as CHO, COS, HEK-293, Jurkat, and MCF-7; replication incompetent adenoviral vector vectors pAdeno X, pAd5F35, pLP-Adeno-X-CMV (Clontech), pAd/CMV/V5-DEST, pAd-DEST vector (Invitrogen) for adenovirus-mediated gene transfer and expression in mammalian cells; pLNCX2, pLXSN, and pLAPSN retrovirus vectors for use with the Retro-X™ system from Clontech for retroviral-mediated gene transfer and expression in mammalian cells; pLenti4V5-DEST.TM., pLenti6/V5-DEST™, and pLenti6.2/V5-GW/lacZ (Invitrogen) for lentivirus-mediated gene transfer and expression in mammalian cells; adenovirus-associated virus expression vectors such as pAAV-MCS, pAAV-IRES-hrGFP, and pAAV-RC vector (Stratagene) for adeno-associated virus-mediated gene transfer and expression in mammalian cells; BACpak6 baculovirus (Clontech) and pFastBac™-HT (Invitrogen) for the expression in Spodopera frugiperda 9 (S9) and Sfll insect cell lines; pMT/BiP/V5-His (Invitrogen) for the expression in Drosophila Schneider S2 cells; Pichia expression vectors pPICZa, pPICZ, pFLDa and pFLD (Invitrogen) for expression in Pichia pastoris and vectors pMETa and pMET for expression in P. methanolica; pYES2/GS and pYDI (Invitrogen) vectors for expression in yeast Saccharomyces cerevisiae. Recent advances in the large scale expression heterologous proteins in Chlamydomonas reinhardtii are described by Griesbeck C. et. al. Mol. Biotechnol. 34:213-33 (2006) and Fuhrmann M., Methods Mol Med. 94:191-5 (2004).

Besides cell-based expression systems, cell-free expression systems are also contemplated. Cell-free expression systems offer several advantages over traditional cell-based expression methods, including the easy modification of reaction conditions to favor protein folding, decreased sensitivity to product toxicity and suitability for high-throughput strategies such as rapid expression screening or large amount protein production because of reduced reaction volumes and process time. The cell-free expression system can use plasmid or linear DNA. Moreover, improvements in translation efficiency have resulted in yields that exceed a milligram of protein per milliliter of reaction mix.

In some embodiments, a continuous cell-free translation system can be used to produce the fusion protein or a fragment or a variant thereof. A continuous cell-free translation system capable of producing proteins in high yield is described by Spirin A S. et. al., Science 242:1162 (1988). The method uses a continuous flow design of the feeding buffer which contains amino acids, adenosine triphosphate (ATP), and guanosine triphosphate (GTP) throughout the reaction mixture and a continuous removal of the translated polypeptide product. The system uses E. coli lysate to provide the cell-free continuous feeding buffer. This continuous flow system is compatible with both prokaryotic and eukaryotic expression vectors. Large scale cell-free production is described by Chang G. el. al., Science 310:1950-3 (2005).

Other commercially available cell-free expression systems include the Expressway™ Cell-Free Expression Systems (Invitrogen), which utilize an E. coli-based in vitro system for efficient, coupled transcription and translation reactions to produce up to milligram quantities of active recombinant protein in a tube reaction format; the Rapid Translation System (RTS) (Roche Applied Science), which also uses an E. coli-based in vitro system; and the TNT Coupled Reticulocyte Lysate Systems (Promega), which uses rabbit reticulocyte-based in-vitro system.

In other exemplary production methods, the fusion protein or a fragment or a variant thereof described herein can be synthesized de novo in whole or in part, using chemical methods. For example, the component amino acid sequences of the fusion protein or a fragment or a variant thereof can be synthesized by solid phase techniques, cleaved from the resin, and purified by preparative high performance liquid chromatography followed by chemical linkage to form a desired polypeptide. The composition of the synthetic peptides may be confirmed by amino acid analysis or sequencing.

Methods for detecting and/or measuring the amount of endotoxin present in a sample (both before and after purification) can be based on commercial kits that are available. For example, the concentration of endotoxin in a protein sample can be determined using the QCL-1000 Chromogenic kit (BioWhittaker), the limulus amebocyte lysate (LAL)-based kits such as the Pyrotell®, Pyrotell®-T, Pyrochrome®, Chromo-LAL, and CSE kits available from the Associates of Cape Cod Incorporated. In some embodiments, endotoxins can be removed from the fusion protein preparations using a variety of commercially available reagents including, without limitation, the ProteoSpin™ Endotoxin Removal Kits (Norgen Biotek Corporation), Detoxi-Gel Endotoxin Removal Gel (Thermo Scientific; Pierce Protein Research Products), MiraCLEAN® Endotoxin Removal Kit (Mirus), or Acrodisc™-Mustang® E membrane (Pall Corporation).

In some embodiments, following expression and purification, the fusion protein or a fragment or a variant thereof described herein can be modified. The modifications can be covalent or non-covalent modifications. Such modifications can be introduced into the fusion protein or a fragment or a variant thereof by, e.g., reacting targeted amino acid residues of the fusion protein or a fragment or a variant thereof with an organic derivatizing agent that is capable of reacting with selected side chains or terminal residues. Suitable sites for modification can be chosen using any of a variety of criteria including, e.g., structural analysis or amino acid sequence analysis of the fusion protein or a fragment or a variant thereof.

In some exemplary production methods, the fusion protein or a fragment or a variant thereof as described herein can be conjugated to a heterologous moiety. In some embodiments, the heterologous moiety can be, e.g., a heterologous polypeptide, a therapeutic agent (e.g., a toxin or a drug), or a detectable label such as, but not limited to, a radioactive label, an enzymatic label, a fluorescent label, or a luminescent label. Suitable heterologous polypeptides can include, e.g., an antigenic tag (e.g., FLAG, polyhistidine, hemagglutinin (HA), glutathione-S-transferase (GST), or maltose-binding protein (MBP)) for use in purifying antibodies or fragments or variants thereof. Heterologous polypeptides can also include polypeptides that are useful as diagnostic or detectable markers, for example, luciferase, green fluorescent protein (GFP), or chloramphenicol acetyl transferase (CAT). Where the heterologous moiety is a polypeptide, the moiety can be incorporated into the fusion protein or a fragment or a variant thereof described herein, resulting in a fusion protein comprising the heterologous moiety.

### Promoters

“Promoter” refers to a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. Yet other promoters are tissue specific or activated promoters, including but not limited to T-cell specific promoters.

The term “promoter activity” and its grammatical equivalents as used herein refer to the extent of expression of nucleotide sequence that is operably linked to the promoter whose activity is being measured. Promoter activity can be measured directly by determining the amount of RNA transcript produced, for example by Northern blot analysis or indirectly by determining the amount of product coded for by the linked nucleic acid sequence, such as a reporter nucleic acid sequence linked to the promoter.

“Inducible promoter” as used herein refers to a promoter which is induced into activity by the presence or absence of transcriptional regulators, e.g., biotic or abiotic factors. Inducible promoters are useful because the expression of genes operably linked to them can be turned on or off at certain stages of development of an organism or in a particular tissue. Examples of inducible promoters are alcohol-regulated promoters, tetracycline-regulated promoters, steroid-regulated promoters, metal-regulated promoters, pathogenesis-regulated promoters, temperature-regulated promoters and light-regulated promoters.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences can also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the promoter is a constitutive promoter, a tissue specific promoter, or an inducible promoter. In some embodiments, the inducible promoter is a small molecule ligand -inducible two polypeptide ecdysone receptor-based gene switch.

Additional promoter elements, e.g., enhancers, can regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it can appear that individual elements can function either cooperatively or independently to activate transcription.

### Reporters

Reporter genes can be used for identifying potentially transfected cells and for evaluating the functionality of regulatory sequences. In general, a reporter gene is a gene that is not present in or expressed by the host cell and that encodes a polypeptide whose expression is manifested by some easily detectable property, e.g., enzymatic activity. Expression of the reporter gene is assayed at a suitable time after the polypeptides encoding the fusion protein or a fragment or a variant thereof has been introduced into the host cells. Suitable reporter genes can include, but not limited to, genes encoding luciferase, beta-galactosidase, chloramphenicol acetyl transferase, secreted alkaline phosphatase, or the green fluorescent protein gene (e.g., Ui-Tei et al., FEBS Letters 479:79-82 (2000)). Suitable expression systems are well known and can be prepared using known techniques or obtained commercially. In general, a polynucleotide construct with the minimal 5′ flanking region showing the highest level of expression of reporter gene is identified as the promoter. Such promoter regions can be linked to a reporter gene and used to evaluate agents for the ability to modulate promoter-driven transcription.

### Fragments and Variants

The term “fragment” or “variant” refers to variants and derivatives of the fusion protein described herein, containing one or more binding moieties to e.g., PD-1, MUCI, MUC16 and/or TGF-β. In certain embodiments, amino acid sequence variants of the fusion protein are contemplated. For example, in some embodiments, amino acid sequence variants of the fusion protein described herein are contemplated to improve the binding affinity and/or other biological properties of the fusion protein. Exemplary method for preparing amino acid variants include, but are not limited to, introducing appropriate modifications into the nucleotide sequence encoding the fusion protein, or by peptide synthesis. Such modifications include, for example, deletions from, and/or insertions into and/or substitutions of residues within the amino acid sequences of the fusion protein.

Any combination of deletion, insertion, and substitution can be made to the various domains to arrive at the final fusion protein, provided that the final fusion protein possesses the desired characteristics, e.g., PD-1 inhibition, MUC1/16 binding ability, and TGF-β trap. In some embodiments, fusion protein variants having one or more amino acid substitutions are provided. In some embodiments, sites of interest for substitution mutagenesis can include the CDRs and framework regions. In some embodiments, amino acid substitutions can be introduced into the variable domains of the target-binding protein of interest and the products screened for a desired activity, e.g., retained/improved antigen binding, decreased immunogenicity, or improved antibody-dependent cell mediated cytotoxicity (ADCC) or complement dependent cytotoxicity (CDC). Both conservative and non-conservative amino acid substitutions can be contemplated for preparing the antibody variants (e.g., anti-PD-1 antibody variants).

In another example of a substitution to create a variant fusion protein can be substituting one or more hypervariable region residues in a parent antibody. In general, variants are then selected based on improvements in desired properties compared to a parent antibody, for example, increased affinity, reduced affinity, reduced immunogenicity, increased pH dependence of binding. For example, an affinity matured variant antibody can be generated, e.g., using phage display-based affinity maturation techniques, such as those described herein and known in the field. Substitutions can be made in hypervariable regions (HVR) of a parent antibody to generate variants, and variants are then selected based on binding affinity, i.e., by affinity maturation. In some embodiments of affinity maturation, diversity can be introduced into the variable genes chosen for maturation by any of a variety of methods (e.g., error-prone PCR, chain shuffling, or oligonucleotide-directed mutagenesis). A secondary library can be then created. The library can be then screened to identify any antibody variants with the desired affinity. Another method to introduce diversity can involve HVR-directed approaches, in which several HVR residues (e.g., 4-6 residues at a time) can be randomized. HVR residues involved in antigen binding can be specifically identified, e.g., using alanine scanning mutagenesis or modeling. Substitutions can be in one, two, three, four, or more sites within a parent antibody sequence.

In some embodiments, a fragment or a variant of the fusion protein, as described herein can comprise a VL and a VH domain with amino acid sequences corresponding to the amino acid sequence of a naturally occurring VL or VH domain, respectively, but that has been “humanized,” i.e., by replacing one or more amino acid residues in the amino acid sequence of the naturally occurring VL or VH domains (and in particular in the framework sequences) by one or more of the amino acid residues that occur at the corresponding position(s) in a VL or VH domain from a conventional 4-chain antibody from a human. This can be performed in a manner known in the field, which can be clear to the skilled person, for example on the basis of the further description herein. It should be noted that such humanized fusion protein or a fragment or a variant thereof are obtained in any suitable manner known per se and thus are not strictly limited to polypeptides that have been obtained using a polypeptide that comprises a naturally occurring VL and/or VH domain as a starting material.

In some embodiments, a fragment or a variant of the fusion protein, as described herein, comprises a VL and a VH domain with amino acid sequences corresponding to the amino acid sequence of a naturally occurring VL or VH domain, respectively, but that has been “camelized,” i.e., by replacing one or more amino acid residues in the amino acid sequence of a naturally occurring VL or VH domain from a conventional 4-chain antibody by one or more of the amino acid residues that occur at the corresponding position(s) in a VL or a VH domain of a heavy chain antibody. Such “camelizing” can be preferably inserted at amino acid positions that form and/or are present at the VH-VL interface, and/or at the so-called Camelidae hallmark residues (see for example WO 94/04678 and Davies and Riechmann (1994 and 1996)). In some embodiments, the VH sequence that is used as a starting material or starting point for generating or designing the camelized single domain can be a VH sequence from a mammal, e.g., a human, such as a VH3 sequence. It should be noted that such camelized fusion protein or a fragment or a variant thereof, in certain embodiments, can be obtained in any suitable manner known in the field and thus are not strictly limited to polypeptides that have been obtained using a polypeptide that comprises a naturally occurring VL and/or VH domain as a starting material.

For example, both “humanization” and “camelization” can be performed by providing a nucleotide sequence that encodes a naturally occurring VL and/or VH domain, respectively, and then changing, one or more codons in the nucleotide sequence in such a way that the new polynucleotide sequence encodes a “humanized” or “camelized” fusion protein or a fragment or a variant thereof, respectively. This polynucleotide can then be expressed, so as to provide the desired binding capabilities (e.g., PD-1). Alternatively, in other embodiments, the fusion protein or a fragment or a variant thereof comprises a “humanized” or “camelized” antibody synthesized de novo using known peptide synthesis technique from the amino acid sequence of a naturally occurring antibody comprising a VL and/or VH domain. In some embodiments, the fusion protein or a fragment or a variant thereof comprises a “humanized” or “camelized” antibody synthesized de novo using known peptide synthesis technique from the amino acid sequence or nucleotide sequence of a naturally occurring antibody comprising a VL and/or VH domain, respectively, a nucleotide sequence encoding the desired humanized or camelized antibody of the disclosure, respectively, is designed and then synthesized de novo using known techniques for nucleic acid synthesis, after which the nucleic acid thus obtained is expressed in using known expression techniques, so as to provide the desired antibody.

Other suitable methods and techniques for obtaining the fusion protein or a fragment or a variant thereof, starting from naturally occurring sequences for VL or VH domains, for example, comprises combining one or more parts of one or more naturally occurring VL or VH sequences (such as one or more framework (FR) sequences and/or complementarity determining region (CDR) sequences), and/or one or more synthetic or semi-synthetic sequences, and/or a naturally occurring sequence for a CH2 domain, and a naturally occurring sequence for a CH3 domain comprising amino acid substitutions that favor formation of heterodimer over homodimer, in a suitable manner, so as to provide the fusion protein or a fragment or a variant thereof.

### Antibody Engineering

In some embodiments, it can be desirable to change certain amino acids containing exposed side-chains to another amino acid residue in order to provide for greater chemical stability of a final antibody, as follows. The deamidation of asparagine can occur on N-G or D-G sequences and result in the creation of an isoaspartic acid residue that introduces a kink into the polypeptide chain and decreases its stability (isoaspartic acid effect). In some embodiments, the antibodies of the fusion protein of the present disclosure do not contain asparagine isomerism sites.

For example, an asparagine (Asn) residue may be changed to Gln or Ala to reduce the potential for formation of isoaspartate at any Asn-Gly sequences, particularly within a CDR. A similar problem can occur at a Asp-Gly sequence. Reissner and Aswad (2003) Cell. Mol. Life Sci. 60:1281. Isoaspartate formation can debilitate or completely abrogate binding of an antibody to its target antigen. See, Presta (2005) J. Allergy Clin. Immunol. 116:731 at 734. In some embodiments, the asparagine can be changed to glutamine (Gln). It can also be desirable to alter an amino acid adjacent to an asparagine (Asn) or glutamine (Gln) residue to reduce the likelihood of deamidation, which occurs at greater rates when small amino acids occur adjacent to asparagine or glutamine. Sec, Bischoff & Kolbe (1994) J. Chromatog. 662:261. In addition, any methionine residues (typically solvent exposed Met) in CDRs can be changed to Lys, Leu, Ala, or Phe in order to reduce the possibility that the methionine sulfur would oxidize, which can reduce antigen binding affinity and also can contribute to molecular heterogeneity in the final antibody preparation. Id. In some embodiments, the methionine can be changed to alanine (Ala). Additionally, in order to prevent or minimize potential scissile Asn-Pro peptide bonds, it can be desirable to alter any Asn-Pro combinations found in a CDR to Gln-Pro, Ala-Pro, or Asn-Ala. Antibodies with such substitutions can be subsequently screened to ensure that the substitutions do not decrease the affinity or specificity of the antibody for human PD-L1, or other desired biological activity to unacceptable levels.

An antibody of the fusion protein disclosed herein can also be conjugated to a chemical moiety such as a radionuclide or other detectable label. Radionuclides include ⁹⁹Tc, ⁹⁰Y, ¹¹¹ In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷Cu, ²¹⁷Ci, ²¹¹ At, ²¹²Pb, ⁴⁷Sc, ¹⁰⁹Pd, ²³⁴Th, and ⁴⁰K, ¹⁵⁷Gd, ⁵⁵Mn, ⁵²Tr, and ⁵⁶Fe. Fluorescent or chemilluminescent labels can include fluorophores, such as rare earth chelates, fluorescein and its derivatives, rhodamine and its derivatives, isothiocyanate, phycoerythrin, phycocyanin, allophycocyanin, o-phthaladehyde, fluorescamine, 152Eu, dansyl, umbelliferone, luciferin, luminal label, isoluminal label, an aromatic acridinium ester label, an imidazole label, an acridimium salt label, an oxalate ester label, an acquorin label, 2,3-dihydrophthalazinediones, biotin/avidin, spin labels and stable free radicals.

Any method known in the art for conjugating antibody molecules to the various moieties can be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

### Pharmaceutical Compositions

The present disclosure provides a composition comprising the fusion protein or a fragment or a variant thereof described herein and a carrier therefor (e.g., a pharmaceutically acceptable carrier). The composition desirably is a physiologically acceptable (e.g., pharmaceutically acceptable) composition, which comprises a carrier, preferably a physiologically (e.g., pharmaceutically) acceptable carrier, and the fusion protein or a fragment or a variant thereof. Any suitable carrier can be used within the context of the present disclosure, and such carriers are well known in the art. The choice of carrier will be determined, in part, by the particular use of the composition (e.g., administration to an animal) and the particular method used to administer the composition. Ideally, in the context of replication-deficient adenoviral vectors, the pharmaceutical composition preferably is free of replication-competent adenovirus. The pharmaceutical composition optionally can be sterile.

Suitable compositions include aqueous and non-aqueous isotonic sterile solutions, which can contain anti-oxidants, buffers, and bacteriostats, and aqueous and non-aqueous sterile suspensions that can include suspending agents, solubilizers, thickening agents, stabilizers, and preservatives. The composition can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. In some cases, the fusion protein or a fragment or a variant thereof is part of a composition formulated to protect the fusion protein or a fragment or a variant thereof from damage prior to administration. For example, the composition can be formulated to reduce loss of the fusion protein or a fragment or a variant thereof on devices used to prepare, store, or administer the fusion protein or a fragment or a variant thereof, such as glassware, syringes, or needles. The composition can be formulated to decrease the light sensitivity and/or temperature sensitivity of the fusion protein or a fragment or a variant thereof. To this end, the composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the fusion protein or a fragment or a variant thereof, and facilitate its administration. Formulations for fusion protein or a fragment or a variant thereof-containing compositions are further described in, for example, U.S. Pat. Nos. 6,225,289, 6,514,943, and International Patent Application Publication WO 2000/034444.

The composition also can be formulated to enhance transduction efficiency. In addition, one of ordinary skill in the art will appreciate that the fusion protein or a fragment or a variant thereof can be present in a composition with other therapeutic or biologically-active agents. For example, factors that control inflammation, such as ibuprofen or steroids, can be part of the composition to reduce swelling and inflammation associated with in vivo administration of the fusion protein or a fragment or a variant thereof. Antibiotics, i.e., microbicides and fungicides, can be present to treat existing infection and/or reduce the risk of future infection, such as infection associated with the administration procedures.

In some instances, pharmaceutical compositions comprising the presently described fusion protein or a fragment or a variant thereof are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Pharmaceutical compositions are optionally manufactured in a conventional manner, such as, by way of example only, by means of conventional mixing, dissolving, granulating, dragée-making, levigating, emulsifying, encapsulating, entrapping or compression processes.

In certain embodiments, compositions can also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions can also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical compositions described herein are administered by any suitable administration route, including but not limited to, oral, parenteral (e.g., intravenous, subcutaneous, intramuscular, intracerebral, intracerebroventricular, intra-articular, intraperitoneal, or intracranial), intranasal, buccal, sublingual, or rectal administration routes. In some instances, the pharmaceutical composition is formulated for parenteral (e.g., intravenous, subcutaneous, intramuscular, intracerebral, intracerebroventricular, intra-articular, intraperitoneal, or intracranial) administration.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragées, capsules, delayed release formulations, extended release formulations, pulsatile release formulations, multiparticulate formulations, and mixed immediate release and controlled release formulations. In some embodiments, the pharmaceutical compositions are formulated into capsules. In some embodiments, the pharmaceutical compositions are formulated into solutions (for example, for IV administration). In some cases, the pharmaceutical composition is formulated as an infusion. In some cases, the pharmaceutical composition is formulated as an injection.

The pharmaceutical solid dosage forms described herein optionally include the presently described fusion protein or a fragment or a variant thereof and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating can be provided around the compositions. In some embodiments, the compositions are formulated into particles (for example for administration by capsule) and some or all of the particles can be coated. In some embodiments, the compositions are formulated into particles (for example for administration by capsule) and some or all of the particles can be microencapsulated. In some embodiments, the compositions can be formulated into particles (for example for administration by capsule) and some or all of the particles are not microencapsulated and are uncoated.

In certain embodiments, compositions provided herein can also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, for example, butylated hydroxytoluene (BHT), sodium ascorbate, ascorbic acid, sodium metabisulfite and tocopherol. In certain embodiments, antioxidants enhance chemical stability where required.

Formulations described herein can benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (c) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Vecgum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib and an anticancer agent, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

“Pharmaceutically compatible carrier materials” can include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. Sec, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or croding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants can be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Kits and Articles of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. The label also indicates directions for use of the contents, such as in the methods described herein.

### Use of Fusion Protein

Also disclosed herein is a use of a fusion protein of the present invention, or a polynucleotide encoding the same, in the manufacture of a medicament for use in the treatment of cancer.

Embodiments of the invention (E1-E130) include:

E1. A fusion protein comprising:
(a) a first component that is an antibody, or a fragment or variant thereof; and
(b) a second component that is: (i) a cytokine trap, or fragment or variant thereof; or (ii) an adenosine deaminase (ADA), or fragment or variant thereof.

E2. The fusion protein of embodiment E1, wherein the first and second components are connected by a linker.

E3. The fusion protein of embodiment E1 or E2, wherein the first component is an immunoglobulin G (IgG).

E4. The fusion protein of any one of embodiments E1-E3, wherein the first component is IgG1, IgG2, IgG3, or IgG4.

E5. The fusion protein of any one of embodiments E1-E4, wherein the first component is IgG1 or IgG4.

E6. The fusion protein of any one of embodiments E1-E3, wherein the the first component is a Fab, (Fab)₂, (Fab′)₂, Fv, (Fv)₂, or scFv.

E7. The fusion protein of any one of embodiments E1-E6, wherein the first component comprises a variable region of a heavy chain (VH) and a variable region of a light chain (VL).

E8. The fusion protein of any one of embodiments E1-E7, wherein the first component comprises a fragment crystallizable region (FC).

E9. The fusion protein of any one of embodiments E1-E8, wherein the first component comprises an FC1, FC2, FC3, or FC4 region, or a fragment or variant thereof.

E10. The fusion protein of any one of embodiments E1-E9, wherein the first component comprises an FC1 region.

E11. The fusion protein of any one of embodiments E1-E10, wherein the first component comprises an FC comprising a mutation.

E12. The fusion protein of any one of embodiments E1-E11, wherein the first component comprises a scFv and an FC.

E13. The fusion protein of any one of embodiments E1-E12, wherein the first component binds to a tumor antigen expressed on the surface of a tumor cell.

E14. The fusion protein of any one of embodiments E1-E13, wherein the first component binds to a tumor antigen expressed on the surface of a tumor cell, wherein the tumor antigen is selected from CD19, BCMA, CD23, BAFF-R, GPRC5D, CD44, CAIX, CD5, CD30, CD70, CD44v6, CD44v7, CD44v8, CD174, CD28, CD128, CD138, CS1, CLL-1, L1-CAM, FAP, ROR1, CEA, EGP-2, EGP-40, HER2, HER3, Folate-binding Protein, GD2, GD3, IL-13R-a2, IL-11RD, EphA2, CSPG4, KDR, EDB-F, mesothelin, CD22, EGFR, Folate receptor α, MUC-1, MUC-4, MUC-16, MAGE-A1, h5T4, PSMA, PSCA, GPC3, c-met, TAG-72, EGFR, CD20, EGFRVIII, CD123, and VEGF-R2.

E15. The fusion protein of any one of embodiments E1-E14, wherein the first component binds to a tumor antigen expressed on the surface of a tumor cell, wherein the tumor antigen is a mucin.

E16. The fusion protein of any one of embodiments E1-E15, wherein the first component binds to a tumor antigen expressed on the surface of a tumor cell, and wherein the tumor antigen is selected from MUC1, MUC2, MUC3A, MUC3B, MUC4, MUC5AC, MUC5B, MUC6, MUC7, MUC8, MUC12, MUC13, MUC15, MUC16, MUC17, MUC19, and MUC20 antigen.

E17. The fusion protein of any one of embodiments E1-E16, wherein the first component binds to a tumor antigen expressed on the surface of a tumor cell, and wherein the tumor antigen is MUC16 or MUC1.

E18. The fusion protein of any one of embodiments E1-E17, wherein the first component binds to a tumor antigen expressed on the surface of a tumor cell, and wherein the tumor antigen is MUC16.

E19. The fusion protein of any one of embodiments E1-E18, wherein the first component comprises a V_{L} region comprising a sequence that is at least 80% identical to any one of SEQ ID NOs: 417-428.

E20. The fusion protein of any one of embodiments E1-E19, wherein the first component comprises a V_{H} region comprising a sequence that is at least 80% identical to any one of SEQ ID NOS: 390-403.

E21. The fusion protein of any one of embodiments E1-E20, wherein the first component comprises: (a) a VL region comprising a sequence that is at least 80% identical to any one of SEQ ID NOs: 417-428; and (b) a VH region comprising a sequence that is at least 80% identical to any one of SEQ ID NOs: 390-403.

E22. The fusion protein of any one of embodiments E1-E21, wherein the first component comprises a VH region comprising a sequence that is at least 80% identical to SEQ ID NO: 399.

E23. The fusion protein of any one of embodiments E1-E22, wherein the first component comprises: (a) a VL region comprising a sequence that is at least 80% identical to SEQ ID NO: 426; and (b) a VH region comprising a sequence that is at least 80% identical to any one of SEQ ID NO: 399.

E24. The fusion protein of any one of embodiments E1-E18, wherein the first component comprises a heavy chain sequence that is at least 80% identical to SEQ ID NO: 441.

E25. The fusion protein of any one of embodiments E1-E18, wherein the first component comprises a light chain sequence that is at least 80% identical to SEQ ID NO: 443.

E26. The fusion protein of any one of embodiments E1-E18, E24, and E25, wherein the first component comprises: (a) a heavy chain sequence that is at least 80% identical to SEQ ID NO: 441; and (b) a light chain sequence that is at least 80% identical to SEQ ID NO: 443.

E27. The fusion protein of any one of embodiments E1-E17, wherein the first component binds to a tumor antigen expressed on the surface of a tumor cell, and wherein the tumor antigen is MUC1.

E28. The fusion protein of any one of embodiments E1-E17 and E27, wherein the first component comprises a VL region comprising a sequence that is at least 80% identical to any one of SEQ ID NOs: 449-453.

E29. The fusion protein of any one of embodiments E1-E17, E27, and E28, wherein the first component comprises a VH region comprising a sequence that is at least 80% identical to any one of SEQ ID NOs: 444-448.

E30. The fusion protein of any one of embodiments E1-E17 and E27-E29, wherein the first component comprises: (a) a VL region comprising a sequence that is at least 80% identical to any one of SEQ ID NOs: 449-453; and (b) a VH region comprising a sequence that is at least 80% identical to any one of SEQ ID NOs: 444-448.

E31. The fusion protein of any one of embodiments E1-E17 and E27-E30, wherein the first component comprises a VH region comprising a sequence that is at least 80% identical to SEQ ID NO: 444.

E32. The fusion protein of any one of embodiments E1-E17 and E27-E31, wherein the first component comprises a heavy chain sequence that is at least 80% identical to SEQ ID NO: 466.

E33. The fusion protein of any one of embodiments E1-E17 and E27-E32, wherein the first component comprises a light chain sequence that is at least 80% identical to SEQ ID NO: 467.

E34. The fusion protein of any one of embodiments E1-E17 and E27-E33, wherein the first component comprises: (a) a heavy chain sequence that is at least 80% identical to SEQ ID NO: 466; and (b) a light chain sequence that is at least 80% identical to SEQ ID NO: 467.

E35. The fusion protein of any one of embodiments E1-E12, wherein the first component binds to programmed cell death protein-1 (PD-1).

E36. The fusion protein of embodiment E35, wherein the first component is an IgG4 antibody comprising a sequence having at least 80% sequence identity to SEQ ID NO: 146 or 292 and having a mutation at position 108 thereof.

E37. The fusion protein of embodiment E35 or E36, wherein the first component is an IgG4 antibody comprising a sequence having at least 80% sequence identity to SEQ ID NO: 146 or 292 and having a S108P mutation.

E38. The fusion protein of embodiment E35, wherein the first component comprises a variable region of a heavy chain (VH) and is connected to the second component by a linker.

E39. The fusion protein of embodiment E35 or E36, wherein the first component comprises a variable region of a light chain (VL) and is connected to the second component by a linker.

E40. The fusion protein of embodiment E38 or E39, wherein the first component comprises a VH connected to a VL by a second linker.

E41. The fusion protein of embodiment E40, wherein the second linker comprises a sequence of any one of SEQ ID NOs: 17-34.

E42. The fusion protein of any one of embodiments E35 and E38-E41, wherein the first component comprises a VH comprising a sequence that is at least 80% identical to any one of SEQ ID NOs: 1-7, 149-164, 333-337, and 384.

E43. The fusion protein of any one of embodiments E35 and E38-E42, wherein the first component comprises a VL comprising a sequence that is at least 80% identical to any one of SEQ ID NOs: 8-13, 148, 338-343, and 385.

E44. The fusion protein of any one of embodiments E35 and E38-E43, wherein the first component comprises: (a) a VH comprising a sequence that is at least 90% identical to SEQ ID NO: 6; and (b) a VL comprising a sequence that is at least 90% identical to SEQ ID NO: 12.

E45. The fusion protein of any one of embodiments E35 and E38-E43, wherein the first component comprises: (a) a VH comprising a sequence that is at least 90% identical to SEQ ID NO: 7; and (b) a VL comprising a sequence that is at least 90% identical to SEQ ID NO: 13.

E46. The fusion protein of any one of embodiments E35 and E38-E43, wherein the first component comprises: (a) a VH comprising a sequence that is at least 90% identical to SEQ ID NO: 333; and (b) a VL comprising a sequence that is at least 90% identical to SEQ ID NO: 338.

E47. The fusion protein of any one of embodiments E35 and E38-E43, wherein the first component comprises: (a) a VH comprising a sequence that is at least 90% identical to SEQ ID NO: 334; and (b) a VL comprising a sequence that is at least 90% identical to SEQ ID NO: 339.

E48. The fusion protein of any one of embodiments E35 and E38-E43, wherein the first component comprises: (a) a VH comprising a sequence that is at least 90% identical to SEQ ID NO: 335; and (b) a VL comprising a sequence that is at least 90% identical to SEQ ID NO: 340.

E49. The fusion protein of any one of embodiments E35 and E38-E43, wherein the first component comprises: (a) a VH comprising a sequence that is at least 90% identical to SEQ ID NO: 336; and (b) a VL comprising a sequence that is at least 90% identical to SEQ ID NO: 341.

E50. The fusion protein of any one of embodiments E35 and E38-E43, wherein the first component comprises: (a) a VH comprising a sequence that is at least 90% identical to SEQ ID NO: 337; and (b) a VL comprising a sequence that is at least 90% identical to SEQ ID NO: 342.

E51. The fusion protein of any one of embodiments E35 and E38-E43, wherein the first component comprises: (a) a VH comprising a sequence that is at least 90% identical to SEQ ID NO: 335; and (b) a VL comprising a sequence that is at least 90% identical to SEQ ID NO: 343.

E52. The fusion protein of any one of embodiments E35 and E38-E43, wherein the first component comprises: (a) a VH comprising a sequence that is at least 90% identical to SEQ ID NO: 384; and (b) a VL comprising a sequence that is at least 90% identical to SEQ ID NO: 385.

E53. The fusion protein of any one of embodiments E35 and E38-E43, wherein the first component comprises: (a) a VH comprising a sequence that is at least 90% identical to SEQ ID NO: 386; and (b) a VL comprising a sequence that is at least 90% identical to SEQ ID NO: 387.

E54. The fusion protein of embodiment E35, wherein the first component is selected from: AM-0001, AMP-224, balstilimab, budigalimab, BI 754091, camrelizumab, cemiplimab, cetrelimab, dostarlimab, JTX-4014, MEDI-0680, MGA012, nivolumab, pembrolizumab, pidilizumab, prolgolimab, sasanlimab, sintilimab, spartalizumab, STI-1110, tislelizumab, toripalimab, and zimberelimab; or a fragment or variant thereof.

E55. The fusion protein of embodiment E35 or E54, wherein the first component is selected from: pembrolizumab, nivolumab, zimberelimab and cetrelimab; or a fragment or variant thereof.

E56. The fusion protein of any one of embodiments E35, E54, or E55, wherein the first component is cetrelimab, or a fragment or variant thereof.

E57. The fusion protein of embodiment E35, wherein the first component comprises: (a) a sequence that is at least 90% identical to SEQ ID NO: 15; and (b) a sequence that is at least 90% identical to SEQ ID NO: 16 or 143.

E58. The fusion protein of embodiment E35 comprising: (a) a sequence that is at least 80% identical to SEQ ID NO: 296; and (b) a sequence that is at least 80% identical to any one of SEQ ID NOs: 144, 145, and 295.

E59. The fusion protein of embodiment E35 comprising: (a) a sequence that is at least 80% identical to SEQ ID NO: 13 or 15; and (b) a sequence that is at least 80% identical to SEQ ID NO: 294 or 295.

E60. The fusion protein of any one of embodiments E1-E59, wherein the second component is a cytokine trap.

E61. The fusion protein of any one of embodiments E1-E60, wherein the cytokine trap is a TGF-β cytokine trap.

E62. The fusion protein of any one of embodiments E1-E61, wherein the cytokine trap is a TGF-β cytokine trap comprising: a transforming growth factor receptor (TGFβR), or a fragment or variant thereof; an anti-TGF-β antibody, or an antigen binding fragment or variant thereof; a TGF-β inhibitory peptide or a fragment or variant thereof, and/or a TGF-β antagonistic peptide or a fragment or variant thereof.

E63. The fusion protein of any one of embodiments E1-E62, wherein the cytokine trap is a TGF-β cytokine trap comprising a transforming growth factor beta receptor II (TGFβRII), or a fragment or variant thereof.

E64. The fusion protein of any one of embodiments E1-E63, wherein the cytokine trap is a TGF-β cytokine trap comprising a TGFβR extracellular domain, or a fragment or variant thereof.

E65. The fusion protein of any one of embodiments E1-E64, wherein the cytokine trap is a TGF-β cytokine trap comprising a TGFβRII extracellular domain, or a fragment or variant thereof.

E66. The fusion protein of any one of embodiments E1-E65, wherein the cytokine trap is a TGF-β cytokine trap comprising a TGFβRII extracellular domain, or fragment or variant thereof, comprising a sequence that is at least 80% identical to any one of SEQ ID NOs: 14, 141, and 142.

E67. The fusion protein of any one of embodiments E1-E66, wherein the cytokine trap is a TGF-β cytokine trap comprising a TGFβRII extracellular domain, or fragment or variant thereof, that binds TGF-β1 and/or TGF-β3.

E68. The fusion protein of any one of embodiments E1-E67, wherein the cytokine trap is a TGF-β cytokine trap comprising a TGFβRII extracellular domain, or fragment or variant thereof, that does not bind TGF-β2 or binds TGF-β2 at a lower affinity than it does TGF-β1 and TGF-β3.

E69. The fusion protein of any one of embodiments E1-E68, wherein the cytokine trap is a TGF-β cytokine trap comprising a sequence that is at least 80% identical to any one of SEQ ID NOS: 14, 141, and 142.

E70. The fusion protein of any one of embodiments E1-E69, wherein the cytokine trap is a TGF-β cytokine trap comprising a sequence that is at least 80% identical to SEQ ID NO: 14.

E71. The fusion protein of any one of embodiments E1-E70, wherein the cytokine trap is a TGF-β cytokine trap comprising an anti-TGF-β antibody or an antigen binding fragment or variant thereof.

E72. The fusion protein of any one of embodiments E1-E71, wherein the cytokine trap is a TGF-β cytokine trap comprising an anti-TGF-β antibody, or an antigen binding fragment or variant thereof, comprising a variable region of a heavy chain (VH) and a variable region of a light chain (VL).

E73. The fusion protein of any one of embodiments E1-E72, wherein the cytokine trap is a TGF-β cytokine trap comprising an anti-TGF-β antibody, or an antigen binding fragment or variant thereof, comprising a VH comprising a sequence that is at least 80% identical to any one of SEQ ID NOs: 166, 168, 169, 171, 173, and 175.

E74. The fusion protein of any one of embodiments E1-E73, wherein the cytokine trap is a TGF-β cytokine trap comprising an anti-TGF-β antibody, or an antigen binding fragment or variant thereof, comprising a VL comprising a sequence that is at least 80% identical to any one of SEQ ID NOs: 165, 167, 170, 172, 174, 176, and 178.

E75. The fusion protein of any one of embodiments E1-E74, wherein the cytokine trap is a TGF-β cytokine trap comprising an anti-TGF-β antibody, or an antigen binding fragment or variant thereof, comprising: (a) a VH comprising a sequence that is at least 80% identical to any one of SEQ ID NOs: 166, 168, 169, 171, 173, and 175; and (b) a VL comprising a sequence that is at least 80% identical to any one of SEQ ID NOs: 165, 167, 170, 172, 174, 176, and 178.

E76. The fusion protein of any one of embodiments E1-E75, wherein the cytokine trap is a TGF-β cytokine trap comprising an anti-TGF-β antibody, or an antigen binding fragment or variant thereof, comprising a VL comprising a sequence that is at least 80% identical to any one of SEQ ID NOs: 165, 167, 170, 172, 174, 176, and 178.

E77. The fusion protein of any one of embodiments E1-E76, wherein the cytokine trap is a TGF-β cytokine trap comprising a TGF-β inhibitory peptide, or a fragment or variant thereof.

E78. The fusion protein of any one of embodiments E1-E77, wherein the cytokine trap is a TGF-β cytokine trap comprising a TGF-β inhibitory peptide, or a fragment or variant thereof, comprising a sequence that is at least 80% identical to any one of SEQ ID NOs: 468-507 and 263-267.

E79. The fusion protein of any one of embodiments E1-E78, wherein the cytokine trap is a TGF-β cytokine trap comprising two or more TGF-β inhibitory peptides, or fragments or variants thereof.

E80. The fusion protein of any one of embodiments E1-E79, wherein the cytokine trap is a TGF-β cytokine trap comprising two or more TGF-β inhibitory peptides, or fragments or variants thereof, that are connected by linker(s).

E81. The fusion protein of any one of embodiments E1-E80, wherein the cytokine trap is a TGF-β cytokine trap comprising a TGF-β antagonistic peptide, or a fragment or variant thereof.

E82. The fusion protein of any one of embodiments E1-E81, wherein the cytokine trap is a TGF-β cytokine trap comprising two or more TGF-β antagonistic peptides, or fragments or variants thereof.

E83. The fusion protein of any one of embodiments E1-E82, wherein the cytokine trap is a TGF-β cytokine trap comprising two or more TGF-β antagonistic peptides, or fragments or variants thereof, that are connected by linker(s).

E84. The fusion protein of any one of embodiments E1-E59, wherein the second component is an ADA, or a fragment or variant thereof.

E85. The fusion protein of any one of embodiments E1-E59, wherein the ADA is adenosine deaminase 2 (ADA2), or a fragment or variant thereof.

E86. The fusion protein of any one of embodiments E1-E59 and E85, wherein the ADA is a fragment or variant of ADA2 that comprises at least one amino acid substitution or deletion.

E87. The fusion protein of any one of embodiments E1-E59, E85, and E86, wherein the ADA comprises a sequence that is at least 80% identical to any one of SEQ ID NOs: 273-279 and 284.

E88. The fusion protein of any one of embodiments E1-E87, wherein the first and second components are connected by a linker comprising (G4S) n, wherein n is 2, 3, 4, 5, or 6 (SEQ ID NO: 555).

E89. The fusion protein of any one of embodiments E1-E88, wherein the first and second components are connected by a linker comprising (Gly) n, wherein n is 6, 7, or 8 (SEQ ID NO: 33).

E90. The fusion protein of any one of embodiments E1-E89, wherein the first and second components are connected by a linker comprising (EAAAK) n, wherein n is 1, 2, 3, 4, 5, or 6 (SEQ ID NO: 34).

E91. The fusion protein of any one of embodiments E1-E90, wherein the first and second components are connected by a linker comprising A (EAAAK)₄ALEA (EAAAK)₄A (SEQ ID NO: 31).

E92. The fusion protein of any one of embodiments E1-E91, wherein the first and second components are connected by a linker comprising a sequence of any one of SEQ ID NOs: E17-E34.

E93. A polynucleotide encoding the fusion protein of any one of embodiments E1-92.

E94. An expression vector comprising the polynucleotide of embodiment E93 operably linked to a promoter.

E95. The vector of embodiment E94, wherein the promoter is constitutive, tissue specific, and/or inducible.

E96. The vector of embodiment E94, wherein the promoter is a small molecule ligand -inducible two polypeptide ecdysone receptor-based gene switch.

E97. The vector of any one of embodiments E94-E96, wherein the vector is an adenoviral vector.

E98. A pharmaceutical composition comprising: (a) the fusion protein of any one of embodiments E1-E92, or a polynucleotide encoding the same; and (b) a pharmaceutically-acceptable excipient.

E99. The pharmaceutical composition of embodiment E98, comprising an expression vector comprising the polynucleotide.

E100. A method of treating cancer, the method comprising contacting a cell with the fusion protein of any one of embodiments E1-E92, or a polynucleotide encoding the same.

E101. The method of embodiment E100, wherein the cell is contacted with an expression vector comprising the polynucleotide.

E102. The method of embodiments E100 or E101, wherein the cell is a cancer cell.

E103. The method of any one of embodiments E100-E102, wherein the cell is a mammalian cell.

E104. A method of treating cancer in a subject in need of such treatment, the method comprising administering to the subject the pharmaceutical composition of embodiments E98 or E99.

E105. The method of embodiment E104, wherein the cancer is a refractory cancer.

E106. The method of embodiment E104 or E105, wherein the subject is non-responsive to treatment with an anti-PD-1 antibody or a CTLA-4 antibody.

E107. The method of any one of embodiments E104-E106, further comprising administering one or more additional anti-cancer agents.

E108. The method of any one of embodiments E104-E107, further comprising administering a PD-1 inhibitor, PD-L1 inhibitor, and/or a CTLA-4 inhibitor.

E109. The method of embodiment E108, wherein the PD-1 inhibitor is an anti-PD-1 antibody, or a fragment or variant thereof.

E110. The method of embodiment E108, wherein the CTLA-4 inhibitor is an anti-CTLA-4 antibody, or a fragment or variant thereof.

E111. The method of any one of embodiments E104-E110, further comprising administering a cytokine.

E112. The method of any one of embodiments E104-E111, further comprising administering a cytokine that is a fusion protein comprising IL-15 and IL-15Rα.

E113. The method of any one of embodiments E104-E112, wherein the subject is a mammal.

E114. The method of any one of embodiments E104-E113, wherein the subject is human.

E115. The method of any one of embodiments E104-E114, wherein the cancer is selected from mesothelioma, glioblastoma, endometrial cancer, colorectal cancer, gastric cancer, cervical cancer, ovarian cancer, pancreatic cancer, prostate cancer, breast cancer, stomach cancer, bladder cancer, liver cancer, Hodgkin's lymphoma, lung cancer, skin cancer, renal cancer, head and neck cancer, melanoma, bronchus cancer, urinary tract cancer, anal cancer, brain cancer, esophageal cancer, cervical cancer, uterine cancer, cancer of the oral cavity or pharynx, kidney cancer, testicular cancer, biliary tract cancer, small bowel cancer, appendix cancer, salivary gland cancer, thyroid gland cancer, adrenal gland cancer, osteosarcoma, chondrosarcoma, and a cancer of a hematological tissue.

E116. The method of any one of embodiments E104-E115, wherein the cancer is cutaneous squamous-cell carcinoma, melanoma, or basal cell cancer.

E117. The method of any one of embodiments E104-E115, wherein the cancer is non-small cell lung cancer (NSLC) or small cell lung cancer (SCLC).

E118. The method of any one of embodiments E104-E115, wherein the cancer is triple negative breast cancer (TNBC).

E119. The method of any one of embodiments E104-E118, further comprising administering an effective amount of T cells engineered to express an exogenous receptor.

E120. The method of embodiment E119, wherein the exogenous receptor is a chimeric antigen receptor.

E121. The method of embodiment E120, wherein the chimeric antigen receptor comprises an antigen binding domain that binds to an epitope on CD19, BCMA, CD23, BAFF-R, GPRC5D, CD44, CAIX, CD5, CD30, CD70, CD44v6, CD44v7, CD44v8, CD174, CD28, CD128, CD138, CS1, CLL-1, L1-CAM, FAP, ROR1, CEA, EGP-2, EGP-40, HER2, HER3, Folate-binding Protein, GD2, GD3, IL-13R-a2, IL-11R, EphA2, CSPG4, KDR, EDB-F, mesothelin, CD22, EGFR, Folate receptor α, MUC-1, MUC-4, MUC-16, MAGE-A1, h5T4, PSMA, PSCA, GPC3, c-met, TAG-72, EGFR, CD20, EGFRVIII, CD123, or VEGF-R2.

E122. The method of embodiment E120 or E121, wherein the chimeric antigen receptor is an engineered T-cell receptor.

E123. The method of any one of embodiments E120-E122, wherein the chimeric antigen receptor comprises an antigen binding domain comprising a sequence that is at least 90% identical to any one of SEQ ID NOs: 37-56.

E124. The method of any one of embodiments E120-E122, wherein the chimeric antigen receptor comprises an antigen binding domain comprising a sequence that is at least 90% identical to SEQ ID NO: 35 or 36.

E125. The method of any one of embodiments E119-E124, wherein the effective amount of engineered T-cells is at least 102 cells/kg.

E126. The method of any one of embodiments E119-E125, wherein the effective amount of engineered T-cells is at least 104 cells/kg.

E127. The method of any one of embodiments E119-E126, wherein the effective amount of engineered T-cells is at least 105 cells/kg.

E128. The method of any one of embodiments E119-E127, wherein the engineered T-cells express a cytokine.

E129. The method of any one of embodiments E119-E128, wherein the engineered T-cells express a cytokine comprising a fusion protein comprising IL-15 and IL-15Rα.

E130. A use of the fusion protein of any one of embodiments E1-E92, or a polynucleotide encoding the same, in the manufacture of a medicament for use in the treatment of cancer.

### EXAMPLES

These examples are provided for illustrative purposes only and do not in any way limit the scope of the claims provided herein.

### EXAMPLE 1. Fusion Protein of Anti-PD-1-TGF-β Trap

Anti-PD-1 VHs and VLs were synthesized and formatted in IgG, scFv-Fc or scFv configurations with TGFβRII fused at C-terminal by linker (G4S)₂ (SEQ ID NO: 563). Anti-PD-1-trap fusion proteins were transiently expressed in Expi293 cells according to manufacturer's protocol. To purify the fusion proteins, the transfected supernatants were loaded to onto a protein L column using AKTA AVANT. The column was washed with PBS, before the protein was eluted with IgG elution buffer (Pierce). The eluted protein was then buffer-exchanged into PBS using a PD-10 column (GE Healthcare).

### Surface Plasmon Resonance

Surface plasma resonance (SPR) analysis was performed using Biacore3000, CM5 chip, an amine-coupling kit, 10×HBS-P running buffer and Glycine for regeneration (GE Healthcare). For anti-PD-1 kinetic assay, purified PD-1 Fc fusion protein was immobilized on CM5 chip using a pre-defined ligand immobilization program. Purified anti-PD-1-Trap fusion proteins were diluted in HBS-P running buffer to a range of final concentrations and injected. Dissociation was allowed to proceed followed by a pulse of 10 mM Glycine (pH 1.5) for regeneration of the chip surface.

To investigate if the anti-PD-1-trap fusion protein could simultaneously bind to PD-1 and TGFβRII, 100 nM of anti-PD-1 trap fusion protein was injected to PD-1 immobilized CM5 chip for 3 minutes, followed by 3 minutes of dissociation. Subsequently, different isoforms of TGF-β were injected to examine the binding of TGF-β to PD-1 bond anti-PD-1-trap fusion protein.

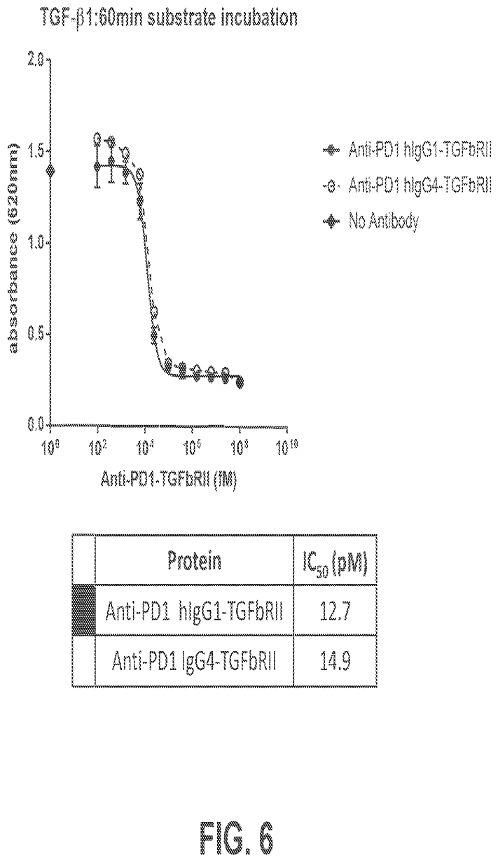

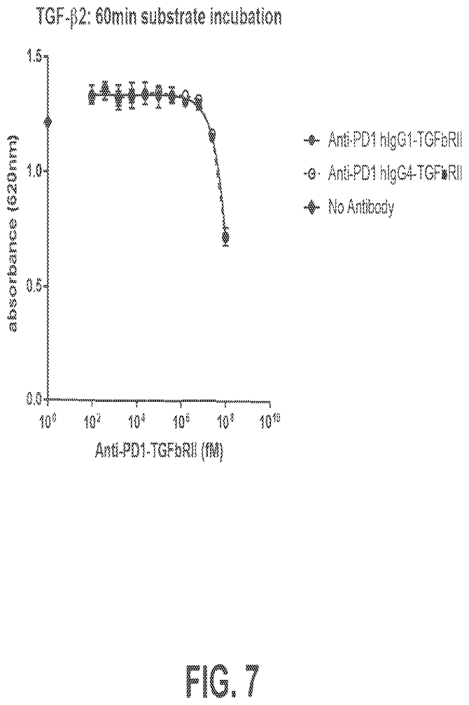

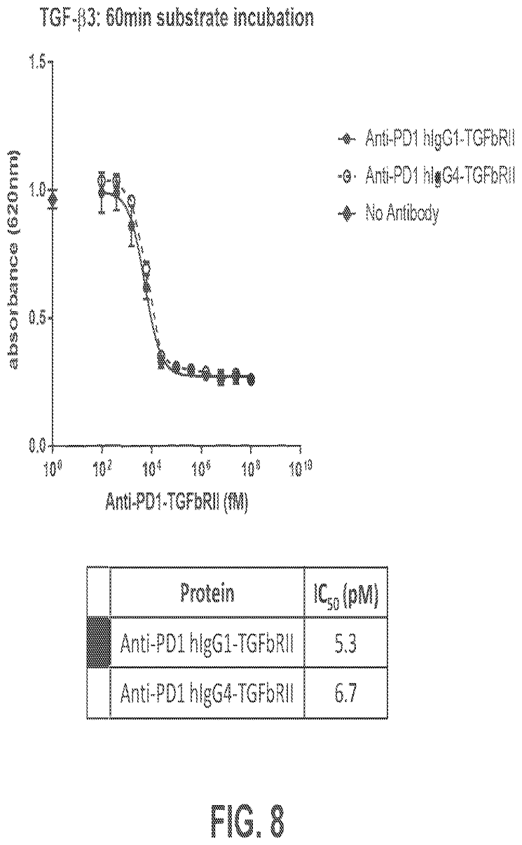

To analyze the binding affinity of anti-PD-1-TGFβRII to TGF-β isoforms, anti-PD-1-TGFβRII were immobilized on CM5 chip and then TGF-β proteins were diluted in HBS-P running buffer to a range of final concentrations and injected to measure the binding affinity of TGF-β to immobilized anti-PD-1-TGFβRII. Affinity of anti-PD-1 (V_{H}6-V_{L}5 or V_{H}7-V_{L}6)-TGFβRII fusion protein to immobilized PD-1 is shown in Table 3 (FIGS. 6-8). Affinity of TGF-β isoforms to immobilized anti-PD-1 IgG4 antibody (V_{H}6-V_{L}5)-TGFβRII fusion protein is shown in Table 4. As observed, the fusion protein bound to TGF-β1 and TGF-β3 with high affinity and bound to TGF-β2 with lower affinity.

### Table 3. Affinity of Anti-PD-1-TGFβRII to Immobilized PD-1

| | K_{D} |
|---|---|
| Anti-PD-1-TGFβRII format | (M) |
| Anti-PD-1(VH7-VL6) IgG4-TGFβRII | 3.79e−11 |
| Anti-PD-1(VH6-VL5) IgG4-TGFβRII | 2.95e−11 |
| Anti-PD-1(VH7-VL6) IgG1-TGFβRII | 8.11e−13 |
| Anti-PD-1(VH6-VL5) IgG1-TGFβRII | 7.64e−12 |
| Anti-PD-1(VH7-VL6) scFv-Fc-TGFβRII | 3.53e−10 |
| Anti-PD-1(VH6-VL5) scFv-Fc-TGFβRII | 2.79e−10 |
| Anti-PD-1(VH7-VL6) scFv-TGFβRII | 7.77e−9 |
| Anti-PD-1(VH6-VL5) scFv-TGFβRII | 1.97e−8 |

Table 4. Affinity of TGF-β to Immobilized Anti-PD-1 (V_{H}6-V_{L}5) IgG4-TGFβRII

| | TGF-β | K_{D} |
|---|---|---|
| Anti-PD-1-TGFβRII | isoform | (M) |
| Anti-PD-1(VH6-VL5) | TGF-β1 | 5.22e−12 |
| IgG4-TGFβRII | TGF-β2 | 8.59e−10 |
| | TGF-β3 | 2.19e−12 |

Binding affinity of various anti-PD-1 variants fused to TGFβRII were assessed by surface plasmon resonance (SPR) assay using Biacore 3000. PD-1 fused to human Fc region was immobilized on sensor chip CM5. Different concentrations of various fusion proteins were injected in solution phase and data was analyzed using BIAevaluation to calculate KD of the fusion proteins. As shown in Table 5, variants such as anti-PD-1 (V_{H}6-V_{L}5)-TGFβRII fusion protein and anti-PD-1 (V_{H}7-V_{L}6)-TGFβRII fusion protein were found to have similar binding affinity to PD-1 compared to respective anti-PD-1 alone.

### Table 5. Affinity of Two Anti-PD-1-TGFβRII Fusion Proteins as Compared to the Respective Anti-PD-1 Alone

| | Binding affinity to |
|---|---|
| | PD-1-Fc antigen |
| Fusion Protein | (K_{D}, M) |
| Anti-PD-1(VH6-VL5)-TGFβRII | 3.42E−13 |
| Anti-PD-1(VH6-VL5) | 4.74E−13 |
| Anti-PD-1(VH7-VL6)-TGFβRII | 6.69E−13 |
| Anti-PD-1(VH7-VL6) | 2.44E−13 |

### PD-1/PD-L1 Blockade Assay

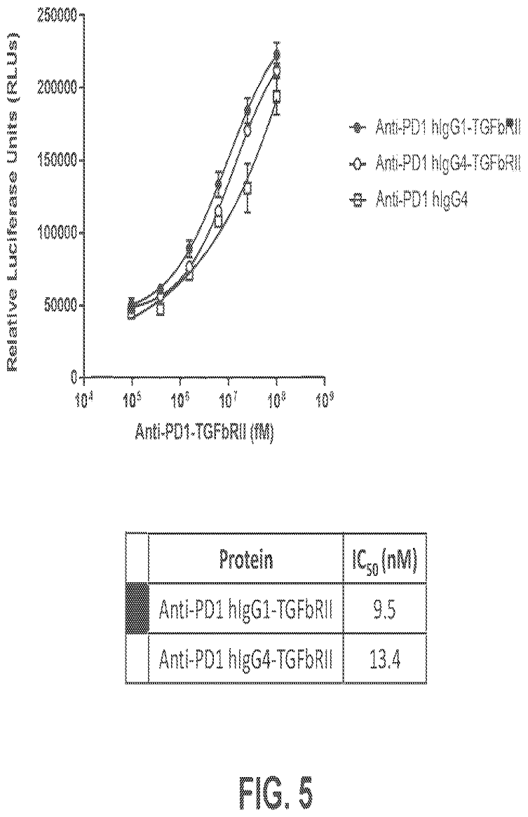

To investigate if the anti-PD-1-TGFβRII fusion proteins could function as an anti-PD-1 by blocking the PD-1/PD-L1 interaction, a PD-1/PD-L1 blockade bioassay kit from Promega was employed. In brief, the PD-L1 aAPC/CHO-K1 cells were thawed one day prior to the start of the assay and allowed to recover overnight at 37° C., 5% CO₂ for 16 to 20 hours. To generate a dose response curve, serial dilutions of anti-PD-1 TGFβRII proteins ranging from 100 nM to 0.098 nM were prepared. The plates were then removed from the incubator and cell media was discarded. Antibodies of interest were then added directly to the plate containing PD-L1 aAPC/CHO-K1 cells. Following this step, PD-1 effector cells were then thawed and added to the 96-well plate. Plates were incubated for at 37° C., 5% CO₂ for 6 hours. Bio-Glo™ luciferase substrate reagent (Promega) was added to each well and Relative Luciferase Units (RLUs) were measured on a Glomax 96-microplate luminometer. The IC₅₀ of the anti-PD-1 TGFβRII proteins were determined by fitting the RLU-concentration data to a four parameter logistic equation. These values were used as a measure of PD-1/PD-L1 blocking potency (FIG. 5).

### HEK-Blue TGF-β Assay

To examine the efficacy of anti-PD-1 TGFβRII fusion proteins to neutralize the function of TGF-β, a stable cell line with induced reporter gene (HEK-Blue™ TGB-β reporter cell line, Invitrogen) was utilized. This stable cell line allows for the detection of bioactive TGF-β via the activation of TGF-β/SMAD pathway, leading to the production of secreted embryonic alkaline phosphatase (SEAP), which can be quantified. In brief, TGF-β1, TGF-2, and TGF-β3 were diluted to a pre-determined EC₅₀ concentration and added to a flat bottom 96-well plate. To generate a dose response curve, serial dilutions of anti-PD-1 TGFβRII proteins were then added to the plate containing different isoforms of TGF-β. Plates were incubated for 30 minutes at 37° C. The HEK-Blue™ TGF-β cells were then added to the plate at a final concentration of 0.7×10⁶ cells/mL. Plates were then incubated at 37° C., 5% CO₂ for a total of 20 hours. Following the incubation, an aliquot of supernatant was removed and added to a plate containing the QUANTI-Blue™ substrate (Invitrogen) used to quantify secreted SEAP. Plates were incubated for one hour at 37° C. The plates were then read on a SpectraMax Plus plate reader at 620 to 655 nm. The IC₅₀ was determined by fitting absorbance-concentration data to a four-parameter logistic equation (GraphPad Prism). Results of the efficacy of various anti-PD-1 TGFβRII fusion proteins in comparison to controls are depicted in FIGS. 5-8.

Tables 6 and 7 further lists various fusion protein constructs tested and the inhibition of TGF-β1 induced reported gene activity.

Table 6: Summary of IC₅₀-inhibition of TGF-β1 induced reporter gene activity for various fusion constructs

| | IC₅₀ |
|---|---|
| Fusion Protein | (pM) |
| Anti-PD-1 (VL5-VH6)•IgG4 | 9.0 |
| Anti-PD-1 (VL5-VH6)•IgG4(S108P)•TGFbRII | 9.7 |
| Anti-PD-1 (VL5-VH6) ScFv-Fc | 4.3 |
| Anti-PD-1 (VL6-VH7)•IgG4.TGFbRII | 5.6 |
| Anti-PD-1 (VL6-VH7)•IgG4(S108P)•TGFbRII | 9.3 |
| Anti-PD-1 (nVL1-nVH3)•IgG4-TGFbRII | 10.7 |
| Anti-PD-1 (nVL1-nVH3)•scFv-Fc-TGFbRII | 6.5 |
| Anti-PD-1 (nVL1-nVH7)•IgG4-TGFbRII | 15.1 |
| Anti-PD-1 (nVL1-nVH8)•IgG4-TGFbRII | 54.5 |
| Anti-RSV IgG4-TGFbRII | 8.6 |
| Anti-RSV S108P IgG4-TGFbRII | 6.9 |
| Anti-RSV scFv-Fc4 | 8.9 |

### Table 7: Summary of TGF-β1 Neutralization Activity of Various Fusion Constructs

| | IC₅₀ |
|---|---|
| Fusion Protein | (pM) |
| Anti-PD-1 (VL5/VH6)•IgG4-Linker2-TGFbRII | 1.2 |
| Anti-PD-1 (VL5/VH6)•IgG4-Linker12-TGFbRII | 2.7 |
| Anti-PD-1 (VL5/VH6)•IgG4-Linker7-TGFbRII | 1.1 |
| Anti-PD-1 (VL5/VH6)•IgG4-Lmker8-TGFbRII | 1.2 |
| Anti-PD-1 (VL5/VH6)•IgG4-Linker10-TGFbRII | 1.9 |

PD-1 Binding of Various Anti-PD-1 lgG4-TGFbRII Fusion Constructs

PD-1-Fc antigen and reference antigen was immobilized on Biacore CM5 surface. 6-12 replicated series diluted concentrations of various anti-PD-1 IgG4-TGFbRII (Table 8) were series injected on PD-1-Fc antigen and reference surface. Kinetics data was evaluated for 1:1 model: Langmuir with mass transfer. As observed in in the table, the binding affinity (KD) of Anti-PD-1 IgG4-TGFbRII with PD-1-Fc antigen was between 1012 to 1010 range.

### Table 8: PD-1 Binding Affinity Analysis of PD-1 IgG4-TGFbRII Fusion Proteins

| | K_{D} |
|---|---|
| Fusion Protein | (M) |
| Anti-PD-1 (nVL1-nVH3)•IgG4 | 1.48E−11 |
| Anti-PD-1 (nVL1-nVH3)•IgG4-TGFbRII | 2.77E−11 |
| Anti-PD-1 (nVL1-nVH3)•IgG4(S108P) | 1.41E−11 |
| Anti-PD-1 (nVL1-nVH3)•IgG4-(S108P)-TGFbRII | 8.16E−12 |
| Anti-PD-1 (nVL1-nVH7)•IgG4-TGFbRII | 1.47E−10 |
| Anti-PD-1 (nVL1-nVH8)•IgG4-TGFbRII | 3.21E−10 |

Simultaneous PD-1 and TGF-β1 Binding of Various Anti-PD-1 1gG4-TGFβRII Fusion Constructs

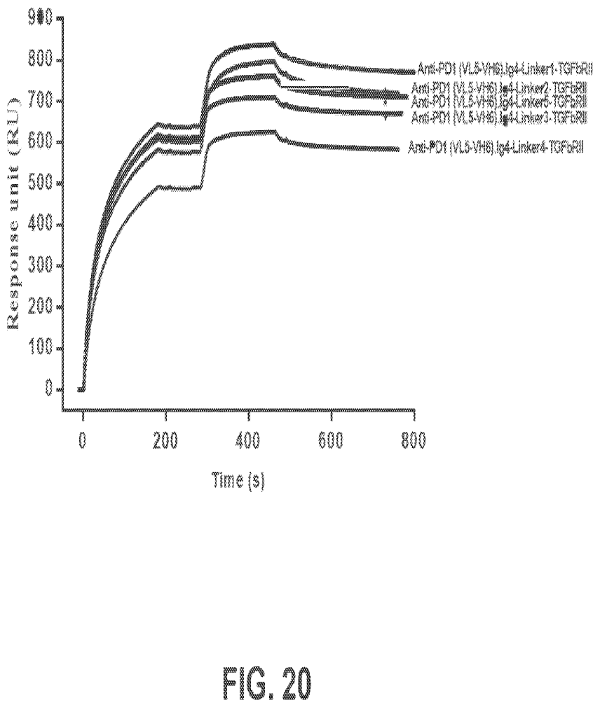

To investigate if the anti-PD-1-TGFRII fusion proteins (Table 9) with different linkers could simultaneously bind to PD-1 and TGF-β1, the following constructs were made and tested in a Biacore experiment. PD-1-Fc antigen was immobilized on a Biacore CM5 chip surface. Anti-PD-1 (VL5-VH6).IgG4-linker-TGFbRII was injected over sensor surfaces, followed by TGFβ1 over sensor surfaces. As shown in FIG. 20, all the anti-PD-1 (VL5-VH6).IgG4-linker-TGFRII constructs bound to both of PD-1-Fc antigen and TGF β1 simultaneously.

Table 9: Anti-PD-1 (VL5-VH6).IgG4-Linker-TGFRII Constructs with Various Linkers

| Fusion protein | Linker |
|---|---|
| Anti-PD-1 (VL5-VH6).IgG4-Linker 2 -TGFbRII | GGGGSGGGS (Linker 2) |
| | (SEQ ID NO. 293) |
| Anti-PD-1 (VL5-VH6).IgG4-Linker12 -TGFbRII | DPVLEREDKVTTSKNPGS (linker 12) |
| | (SEQ ID NO: 27) |
| Anti-PD-1 (VL5-VH6).IgG4-Linker7 -TGFbRII | DPGSGSVPLGSGSNPGS (linker7) |
| | (SEQ ID NO: 22) |
| Anti-PD-1 (VL5-VH6).IgG4-Linker8 -TGFbRII | DPGSGGSVPLGSGGSNPGS (linker 8) |
| | (SEQ ID NO: 23) |
| Anti-PD-1 (VL5-VH6).IgG4-Linker10 -TGFbRII | DPGVLEREDVPTTSYPNPGS (linker10) |
| | (SEQ ID NO: 25) |

### EXAMPLE 2. Anti-PD-1-TGF-β Trap Promotes T Cell Activation in In Vitro Culture

To assess the ability of the anti-PD-1-TGF-β trap to promote T cells functions, peripheral blood mononuclear cells (PBMCs) purified using Ficoll-Hypaque from a leukapheresis product (obtained from a normal healthy donor under an Institutional Review Board (IRB) approved protocol) were used in vitro. PBMCs were labeled with the cell proliferation dye CellTrace violet and stimulated with anti-CD3 and anti-CD28. At the end of the incubation culture supernatants were collected and stored until used for IFN-γ release assay. The cells were labeled with fluorescent conjugated antibodies to assess T cell proliferation.

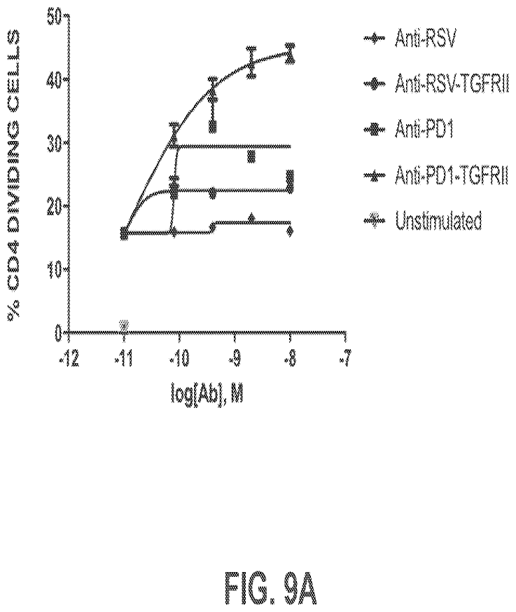

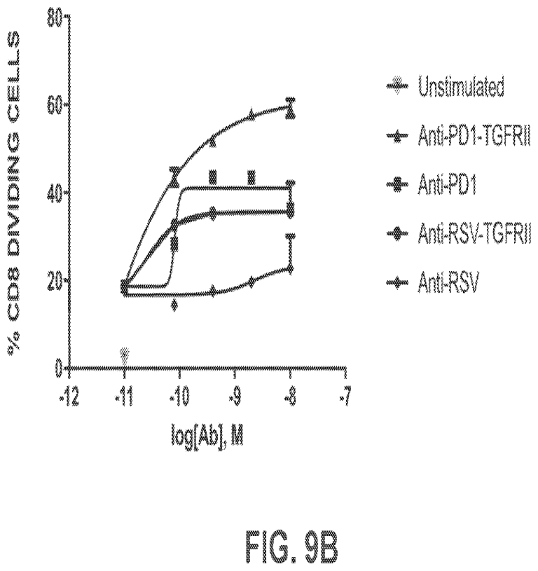

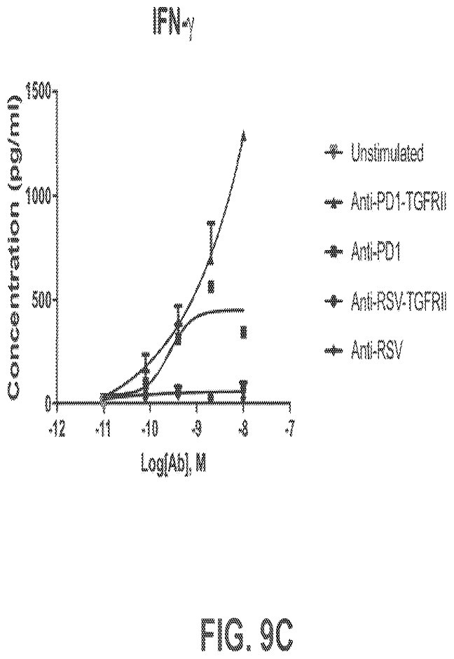

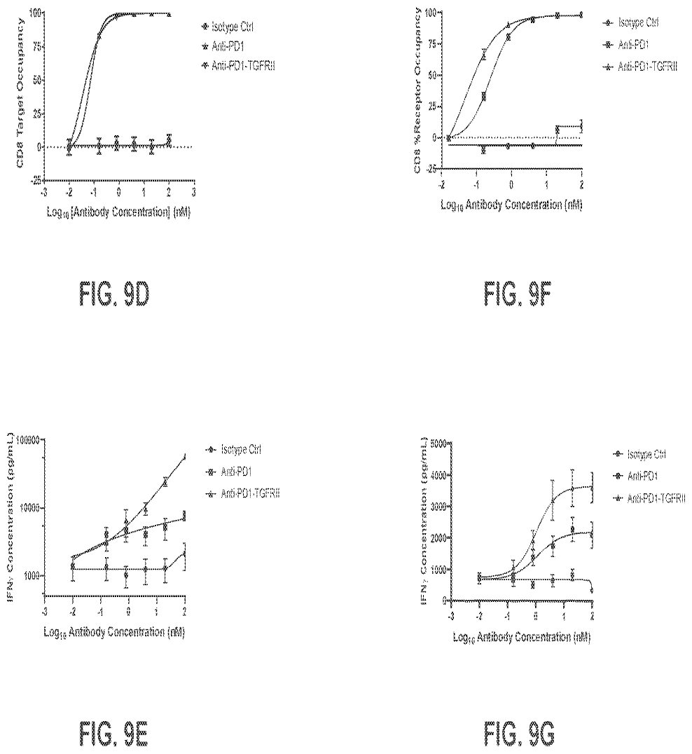

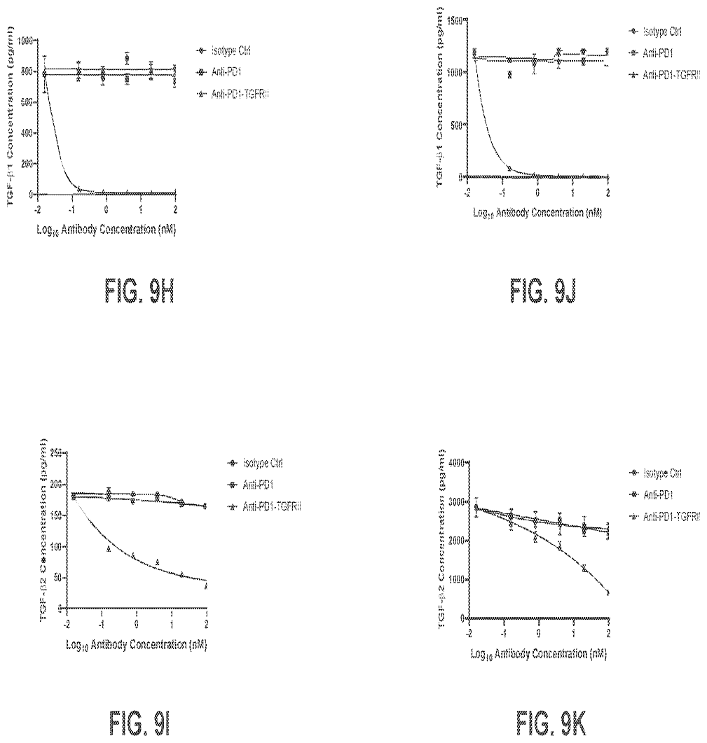

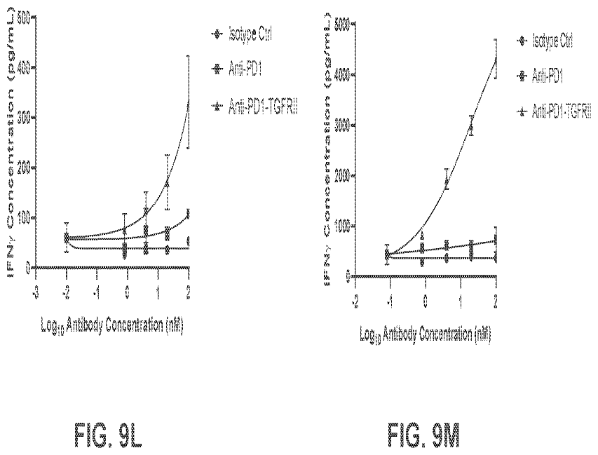

Stimulated PBMCs in the presence of anti-PD-1-TGF-β trap showed enhanced proliferation and IFN-γ production in a dose dependent manner compared to anti-PD-1 or anti-RSV control antibodies suggesting the ability of the fusion molecule to target both the PD-1/PD-L1 and TGF-β pathways (FIGS. 9A-9C). The data also demonstrates an additive enhanced effect of the fusion molecule compared to anti-PD-1 alone.

### Anti PD-1-TGF-RII Exhibits Superior Anti-Tumor Response Compared to Anti PD-1 Alone in an In Vitro Model of Colorectal Cancer

Effectiveness of anti-PD-1-TGFRII fusion protein was evaluated in an in vitro model of colorectal cancer. PBMCs purified from healthy donor were co-cultured with cancer cell line HT-29 for 5 days. The cells were stimulated with anti-CD3 in the presence of isotype control, anti-PD-1 or anti-PD-1-TGFRII fusion protein. The culture supernatants were collected and IFN-γ levels were quantified by MSD per manufacturer's protocol. The cells were collected and target occupancy on T cells were analyzed by flow cytometry. The levels of TGF-β1, TGF-β2 and TGF-β3 in cell culture supernatants were quantified by Luminex per manufacturer's protocol.

FIG. 9D, shows that anti-PD-1-TGFRII binds to PD-1 on CD8+ T cell surface similar to that of anti PD-1. As shown in FIG. 9E anti-PD-1-TGFRII fusion protein promoted higher level of IFN-γ production compared to anti-PD-1 or isotype control. FIG. 9H. TGF-β1 produced in HT-29 PBMC coculture system is completely neutralized upon anti-PD-1-TGFRII treatment even at the lowest dose tested. FIG. 9I. TGF-β2 produced in HT-29 PBMC coculture system is partially neutralized upon anti-PD-1-TGFRII treatment. Overall, results from this in vitro model exhibit superior anti-tumor response of anti-PD-1-TGFRII fusion protein compared to an anti-PD-1 antibody.

### Anti-PD-1-TGFRII Exhibits Superior Anti-Tumor Response Compared to Anti-PD-1 Alone in an In Vitro Model of Head and Neck Cancer

Effectiveness of anti-PD-1-TGFRII fusion protein was evaluated in an in vitro model of head and neck cancer. PBMCs purified from healthy donor were co-cultured with cancer cell line Detroit 562 for 5 days. The cells were stimulated with anti-CD3 in the presence of isotype control, anti-PD-1 or anti-PD-1-TGFRII fusion protein. The culture supernatants were collected and IFN-γ levels were quantified by MSD per manufacturer's protocol. The cells were collected and target occupancy on T cells were analyzed by flow cytometry. The levels of TGF-β1, TGF-β2 and TGF-β3 in cell culture supernatants were quantified by luminex per manufacturer's protocol.

FIG. 9F, shows that anti-PD-1-TGFRII binds to PD-1 on CD8+ T cell surface similar to that of anti PD-1. As shown in FIG. 9G, anti-PD-1-TGFRII fusion protein promoted higher level of IFN-γ production compared to anti-PD-1 or isotype control. FIG. 9J. TGF-β1 produced in Detroit 562 PBMC coculture system is completely neutralized upon anti-PD-1-TGFRII treatment even at the lowest dose tested. FIG. 9K. TGF-β2 produced in Detroit 562 PBMC coculture system is partially neutralized upon anti-PD-1-TGFRII treatment. Overall, results from this in vitro model exhibit superior anti-tumor response of anti-PD-1-TGFRII fusion protein compared to an anti-PD-1 antibody.

### Anti PD-1-TGF-RII Exhibits Superior Anti-Tumor Response Compared to Anti PD-1 Alone in an In Vitro 3D Tumor Spheroid Model of Colorectal Cancer

Effectiveness of anti-PD-1-TGFRII fusion protein was evaluated in an in vitro 3D tumor spheroid model of colorectal cancer. 3D tumor spheroids were generated by coculturing colorectal cancer cell line HT-29 and a fibroblast cell line. PBMCs purified from healthy donor were then added to spheroid culture. The cells were stimulated with anti-CD3 in the presence of isotype control, anti-PD-1 or anti-PD-1-TGFRII fusion protein for 5 days. The culture supernatants were collected and IFN-γ levels were quantified by MSD per manufacturer's protocol.

As shown in FIG. 9L, anti-PD-1-TGFRII fusion protein promoted higher level of IFN-γ production compared to anti-PD-1 or isotype control. Overall, result from this 3D tumor spheroid model exhibit superior anti-tumor response of anti-PD-1-TGFRII fusion protein compared to an anti-PD-1 antibody.

### Anti-PD-1-TGF-RII Exhibits Superior Anti-Tumor Response Compared to Anti PD-1 Alone in an In Vitro 3D Tumor Spheroid Model of Head and Neck Cancer

Effectiveness of anti-PD-1-TGFRII fusion protein was evaluated in an in vitro 3D tumor spheroid model of colorectal cancer. 3D tumor spheroids were generated by coculturing Detroit 562 cell line and a fibroblast cell line. PBMCs purified from healthy donor were then added to spheroid culture. The cells were stimulated with anti-CD3 in the presence of isotype control, anti-PD-1 or anti-PD-1-TGFRII fusion protein for 5 days. The culture supernatants were collected and IFN-γ levels were quantified by MSD per manufacturer's protocol.

As shown in FIG. 9M, anti-PD-1-TGFRII fusion protein promoted higher level of IFN-γ production compared to anti-PD-1 or isotype control. Overall, result from this 3D tumor spheroid model exhibit superior anti-tumor response of anti-PD-1-TGFRII fusion protein compared to an anti-PD-1 antibody.

### EXAMPLE 3. Anti-PD-1-TGF-β Trap has Superior Activity Compared to Anti-PD-1 in the Presence of Recombinant TGF-β1 In Vitro

To assess the ability of the anti-PD-1-TGF-β trap to promote T cells functions, in an environment with high levels of TGF-β, PBMCs were stimulated as above in the presence of recombinant TGF-β1. At the end of the incubation, culture supernatants were collected and stored until used for cytokine analyses. The cells were labeled with fluorescent conjugated antibodies and assessed for T cell proliferation and the upregulation of IL-2 receptor alpha or CD25 as a readout for activation.

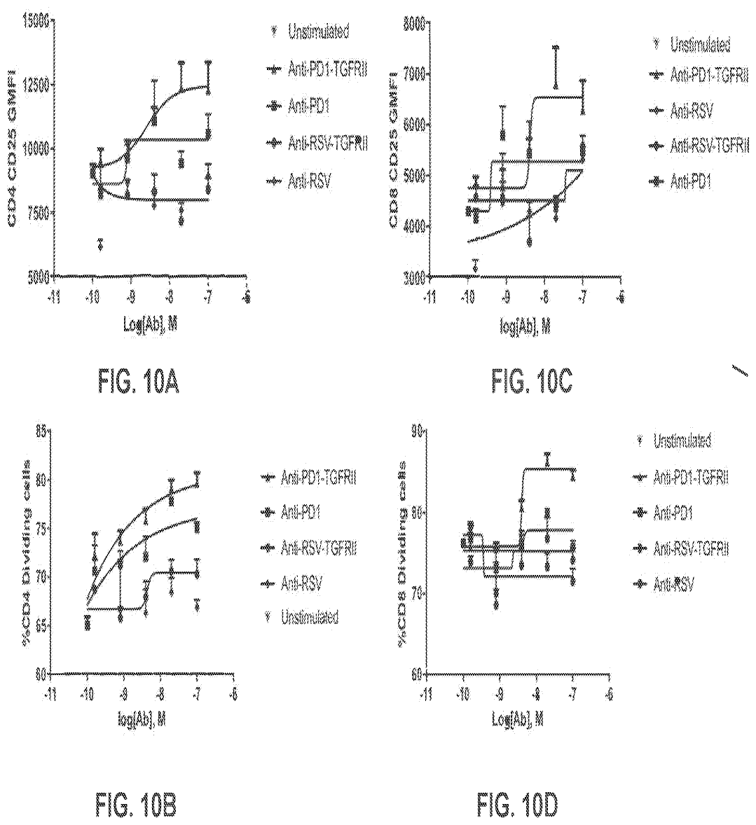

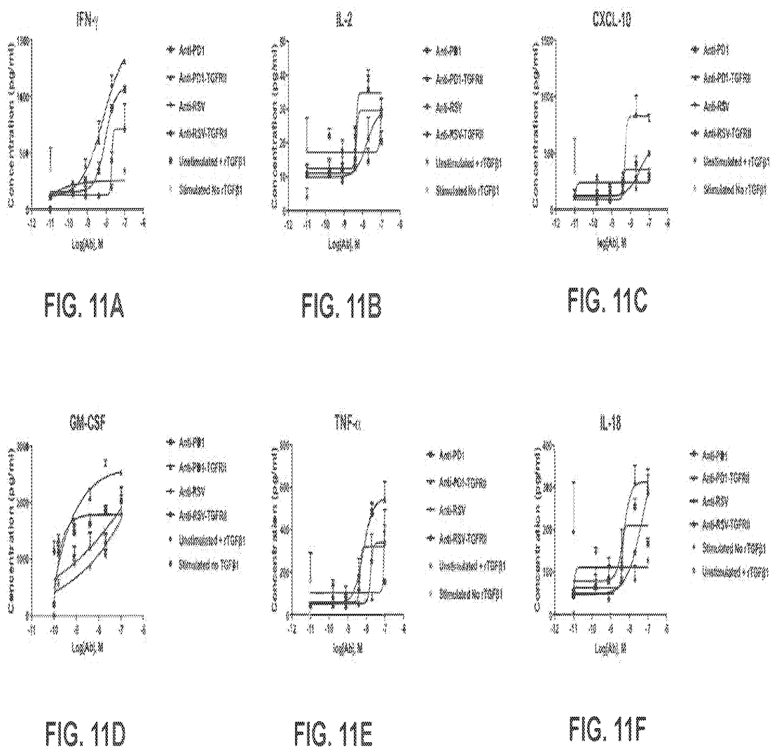

Recombinant TGF-β1 suppressed T cell activation, proliferation and cytokine and chemokine production (FIG. 10 and FIG. 11). This suppression was reversed significantly by the anti-PD-1-TGF-β1 trap in a dose dependent manner compared to anti-PD-1. The data suggests the ability of the fusion protein to neutralize high amount of exogenous TGF-β1 and disrupt the PD-1/PD-L1 pathway resulting in enhance T cell immune responses. Thus, the blockade of the PD-1/PD-L1 and neutralization of TGF-β by the fusion molecule can be an attractive immunotherapy for eliciting potent anti-tumor responses in cancer indications where checkpoint inhibitors treatments have failed.

### EXAMPLE 4. Anti-PD-1-TGF-RII Exhibits Superior Anti-Tumor Response Compared to Anti-PD-1 Alone in a Humanized Mouse Model of Colorectal Cancer

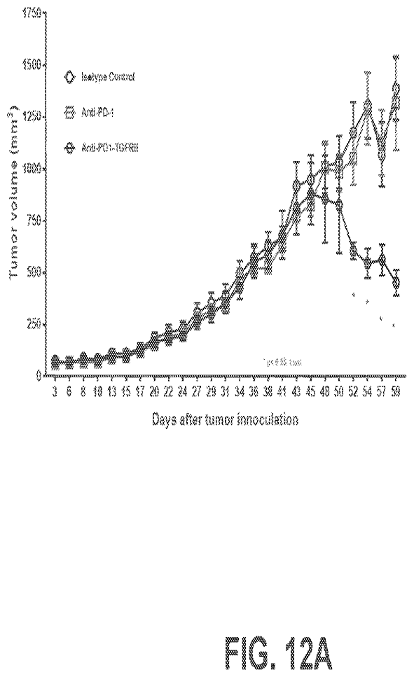

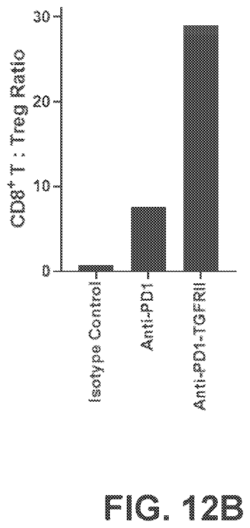

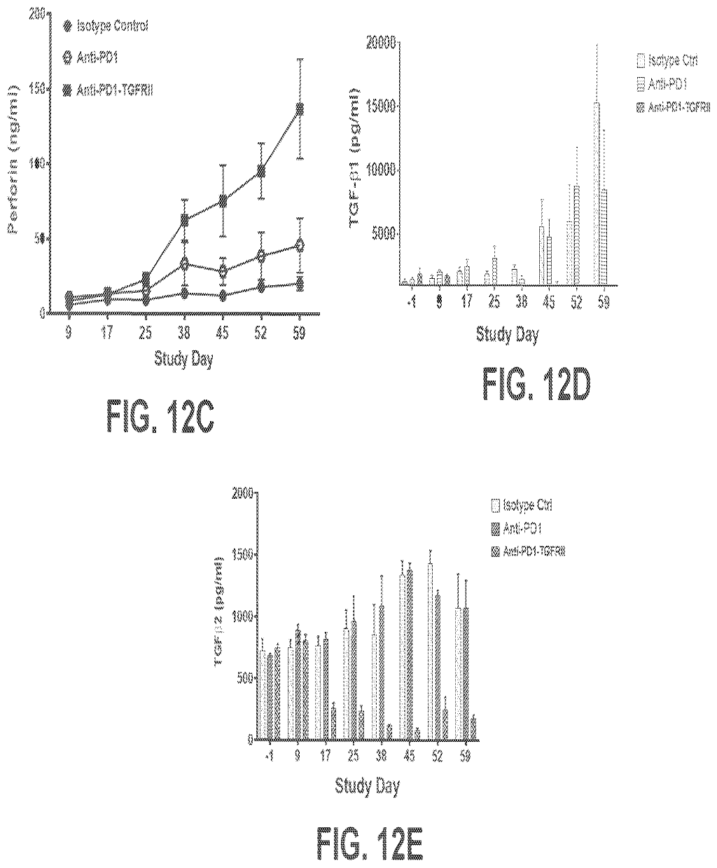

The ability of anti-PD-1-TGFRII fusion protein to inhibit colorectal cancer tumor was evaluated in a humanized mouse model. Humanized NOG mice were administered HT-29 cancer cells. Tumor-bearing mice were randomized and administered twice weekly with either anti-PD-1-TGFRII fusion protein, an anti-PD-1 antibody alone or an isotype control. As seen in the FIG. 12A, anti-PD-1-TGFRII fusion protein significantly inhibited tumor growth compared to anti-PD-1 and the isotype control. Analyses of the tumors at the end of the study showed that anti-PD-1-TGFRII fusion protein treated mice had higher frequency of tumor infiltrating lymphocytes (TILs). Analysis of tumors from anti-PD-1-TGFRII treated mice also showed higher ratio of CD8+ T cells to regulatory T cells (Tregs).

### EXAMPLE 5. Anti-PD-1-TGFRII Exhibits Superior Anti-Tumor Response Compared to Anti-PD-1 Alone in an In Vitro Model of Head and Neck Cancer

Effectiveness of anti-PD-1-TGFRII fusion protein was evaluated in an in vitro model of head and neck cancer. PBMCs purified from healthy donor were co-cultured with cancer cell line Detroit 562 for 48 hours. The cells were stimulated with anti-CD3 in the presence of isotype control, anti-PD-1 or anti-PD-1-TGFRII fusion protein. The culture supernatants were collected and IFN-γ levels were quantified by MSD per manufacture's protocol. The cell pellets from co-culture at the end of culture period were harvested by centrifugation and RNA was purified using the Qiagen RNAeasy micro kit according the manufacturer's protocol. Purified RNA was used for gene expression analysis using the nanostring according to the manufacturer's instructions. Gene expression was analyzed using Nanostring nCounter software.

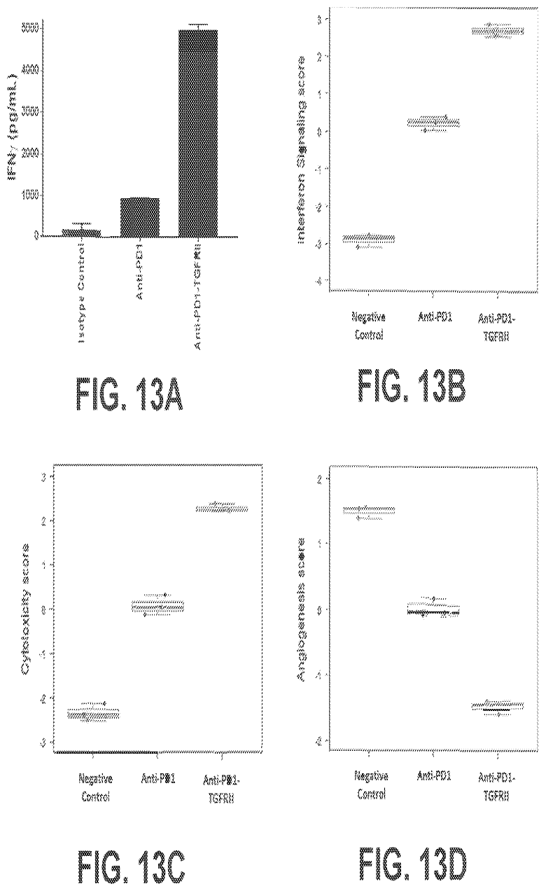

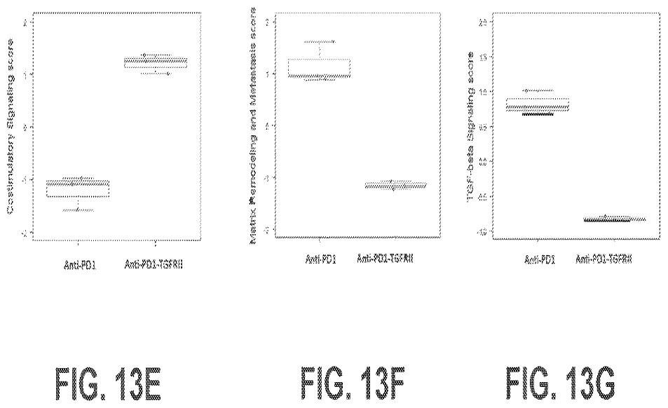

As shown in FIG. 13A, anti-PD-1-TGFRII fusion protein promoted higher level of IFN-γ production compared to anti-PD-1 or isotype control. Anti-PD-1-TGFRII fusion protein treatment resulted in significant upregulation of interferon pathway genes (FIG. 13B) as well as cytotoxic genes (FIG. 13C) compared to anti-PD-1 treatment highlighting improved cytotoxic function in presence of tumor compared to an anti-PD-1 antibody. Furthermore, anti-PD-1-TGFRII fusion protein treatment resulted in significant downregulation of genes involved in tumor metastasis and angiogenesis pathways (FIG. 13D). Overall, results from this in vitro model exhibit superior anti-tumor response of anti-PD-1-TGFRII fusion protein compared to an anti-PD-1 antibody.

### EXAMPLE 6. Anti-PD-1-TGFRII Exhibits Superior Anti-Tumor Response Compared to Anti-PD-1 Alone in a Humanized Mouse Model of Head and Neck Cancer

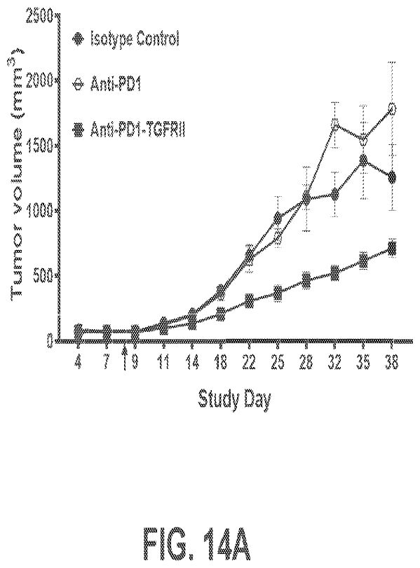

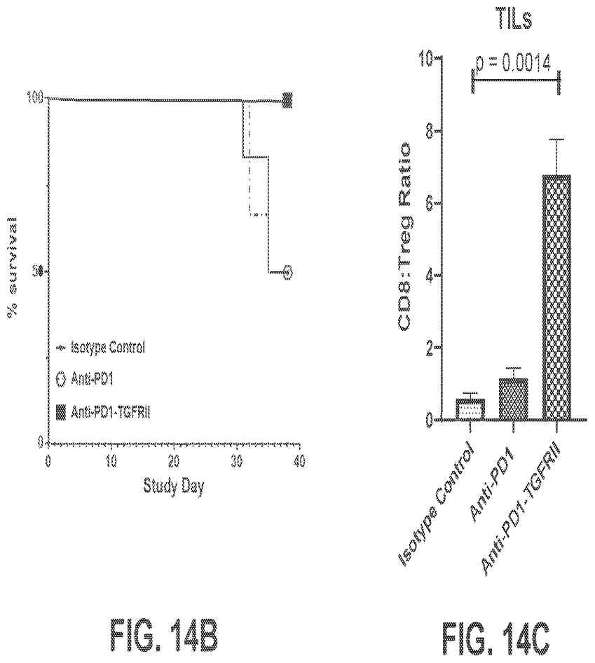

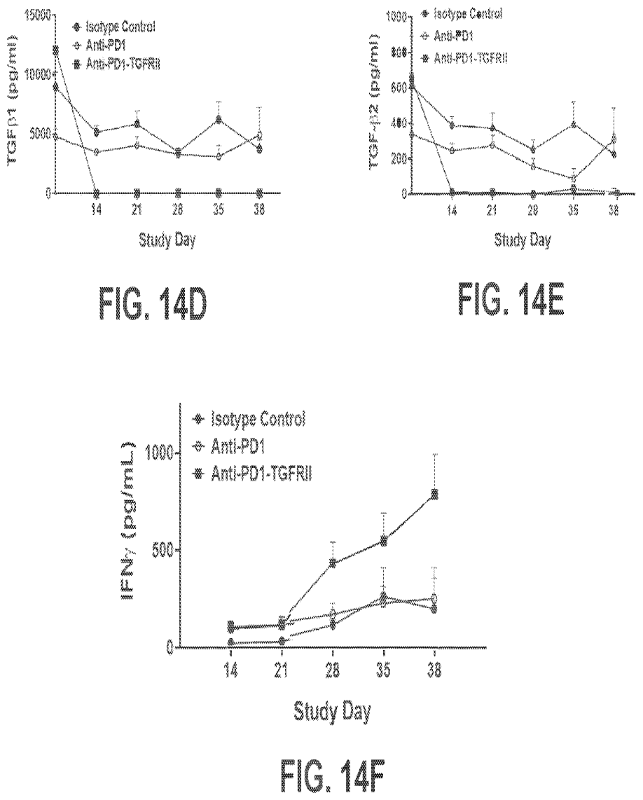

Ability of anti-PD-1-TGFRII fusion protein to inhibit head and neck cancer was evaluated in a humanized mouse model. Humanized NSG mice were administered D562 cell line. Tumor-bearing mice were randomized in and administered twice weekly with anti-PD-1-TGFRII fusion protein, an anti-PD-1 antibody alone or an isotype control. As seen in FIG. 14, Anti-PD-1-TGFRII fusion protein significantly inhibited tumor growth (FIG. 14A), improved survival of tumor bearing mice (FIG. 14B) and increase in IFNγ levels (FIG. 14F) compared to anti-PD-1 and the isotype control. Treatment with anti-PD-1-TGFRII fusion protein significantly improved CD8+ T cells to Treg ratio in tumors (FIG. 14C).

### EXAMPLE 7. Anti-PD-1-TGFRII Exhibits Superior Anti-Tumor Response Compared to Anti-PD-1 Alone in Primary Colorectal Cancer (CRC) Patient Samples

Effectiveness of anti-PD-1-TGFRII fusion protein was evaluated in primary colorectal cancer patient samples. PBMCs from CRC patients were co-cultured with dissociated tumor cells. The cells were stimulated in the presence of isotype control, anti-PD-1 or anti-PD-1-TGFRII fusion protein. The culture supernatants were collected and IFN-γ levels were quantified by MSD per manufacturer's protocol. TGF-β levels were assessed by Luminex per the manufacturer's protocol. The cell pellets from co-culture at the end of culture period were harvested by centrifugation and RNA was purified using the Qiagen RNAeasy micro kit according the manufacturer's protocol. Purified RNA was used for gene expression analysis using the NanoString according to the manufacturer's instructions. Gene expression was analyzed using Nanostring nCounter software.

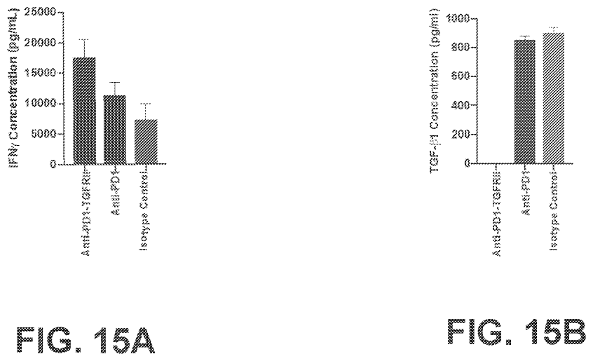

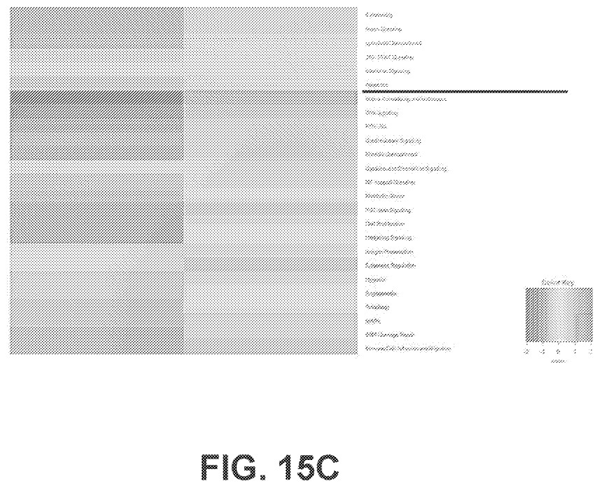

As shown in FIG. 15A, anti-PD-1-TGFRII fusion protein promoted higher level of IFN-γ production compared to anti-PD-1 or isotype control. Anti-PD-TGFRII fusion protein completely neutralized TGF-β1 produced by patient PBMC and dissociated tumor cells (FIG. 15B). Anti-PD-1-TGFRII fusion protein treatment resulted in significant upregulation of cytotoxic genes (FIG. 15C) compared to anti-PD-1 treatment highlighting improved cytotoxic function in presence of tumor compared to an anti-PD-1 antibody. Furthermore, anti-PD-1-TGFRII fusion protein treatment resulted in significant downregulation of genes involved in tumor metastasis and angiogenesis pathways (FIG. 15C). As expected TGF-β signaling pathway is significantly downregulated in anti-PD-1-TGFRII fusion protein treatment group. Overall, results from this patient derived samples exhibit superior anti-tumor response of anti-PD-1-TGFRII fusion protein compared to an anti-PD-1 antibody.

### EXAMPLE 8. Anti-PD-1-TGFRII Exhibits Increased Cytotoxicity Compared to Anti-PD-L1-TGFRII in an In Vitro Model of Ovarian Cancer

Effectiveness of anti-PD-1-TGFRII was compared with anti-PD-L1-TGFRII in an in vitro model of ovarian cancer. PBMCs purified from healthy donors were co-cultured with cancer cell line SK-OV-3. The immune cells were stimulated in the presence of isotype control, anti-PD-1-TGFRII fusion protein or anti-PD-L1-TGFRII fusion protein. The killing of tumor cells was assessed across culture period using Incucyte live cell analysis system.

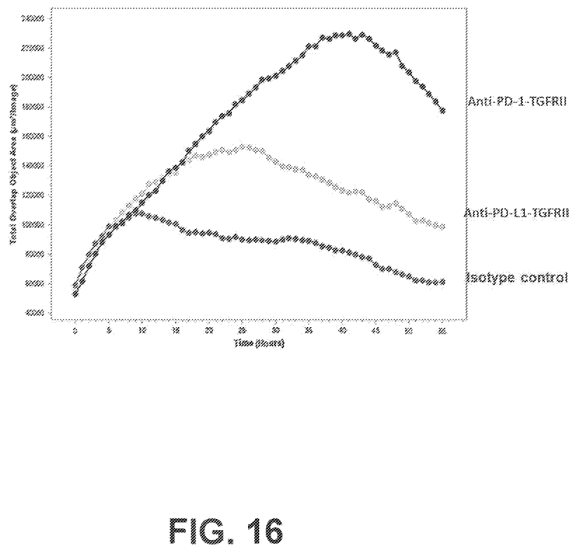

As shown in FIG. 16, the presence of anti-PD-1-TGFRII fusion protein exhibited increased killing of tumor cells when compared to anti-PD-L1-TGFRII fusion protein. Overall, anti-PD-1-TGFRII exhibits improved cytotoxicity when compared to anti-PD-L1-TGFRII.

### EXAMPLE 9. Combination of Anti-PD-1-TGF-RII with CAR-T Significantly Enhanced 3D Tumor Spheroid Killing When Compared to Combination with Anti-PD-1 or CAR-T Alone

Combination of anti-PD-1-TGFRII fusion protein with CAR-T cells was evaluated in an in vitro 3D tumor spheroid model of ovarian cancer. In this example, the CAR-T cells were engineered to express MUC16 CAR. 3D tumor spheroids were generated by coculturing ovarian cancer cell line SK-OV-3 and a fibroblast cell line. CAR-T cells were then added to the 3D tumor spheroid culture in the presence of anti-PD-1-TGFRII fusion protein or anti-PD-1. The target specific killing of 3D tumor spheroid was assessed across time using Incucyte live cell analysis system.

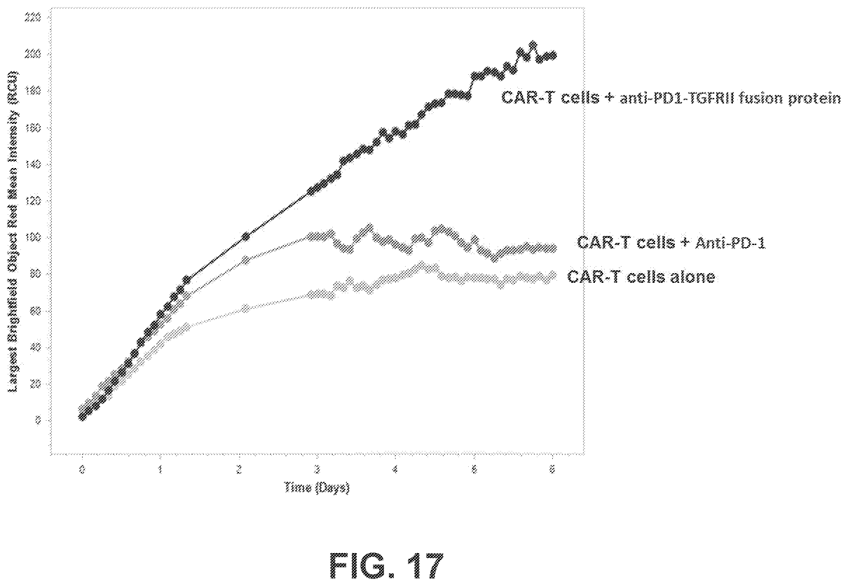

As shown in FIG. 17, the combination of anti-PD-1-TGFRII fusion protein with CAR-T cells showed increased killing of 3-D tumor spheroids when compared to the combination of anti-PD-1 and CAR-T cells or CAR-T cells alone. Overall, result from this kill assay suggest that addition of anti-PD-1-TGF-RII to CAR-T cells enhances the anti-tumor response of CAR-T cells.

### EXAMPLE 10 Combination of Anti-PD-1-TGF-RII with CD33 Specific CAR-T Cells

CAR-T cells were co-cultured with MOLM-13 AML tumor cell line in a cytotoxicity assay and the cultures were incubated in the IncuCyte S3 live cell analysis instrument. In this example, the CAR-T cells were engineered to express CD33 CAR. Anti PD-1 or anti-PD-1-TGFRII was added to the indicated cultures at an equimolar concentration. Sytox Green was directly added at the start of the culture to allow for the identification and enumeration of dying cells in the culture. Analysis was performed using the IncuCyte S3 Software. The data presented in the graph shown is the mean±SD from triplicate wells, with 5 scans/well taken.

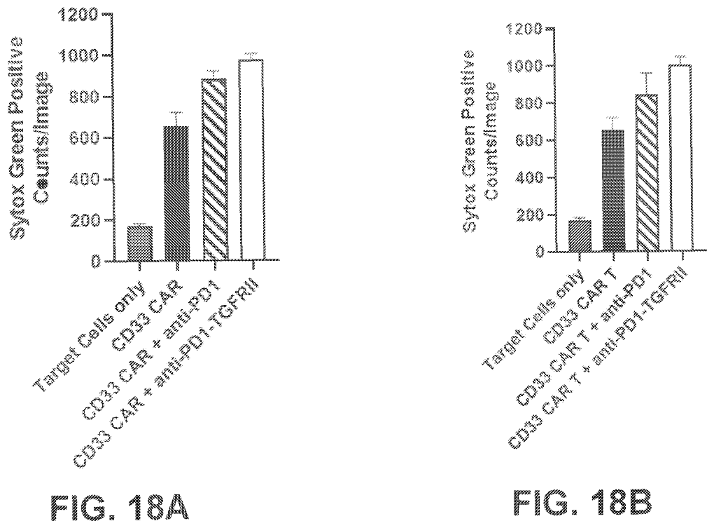

As shown in FIG. 18A, the combination of anti-PD-1 (VH6/VL5)-TGFRII fusion protein with the CD33 CAR T cells showed increased killing as compared to CAR T cells and anti-PD-1 (VH6/VL5) combination or CAR T cells alone.

As shown in FIG. 18B, the combination of anti-PD-1 (VH7/VL6)-TGFRII fusion protein with the CD33 CAR-T cells showed increased killing compared to CAR-T cells and anti-PD-1 combination or CAR-T cells alone. Overall, the result for these cytotoxicity assays demonstrate that anti-PD-1 (VH6/VL5)-TGFRII fusion protein and anti-PD-1 (VH7/VL6)-TGFRII fusion protein enhance the anti-tumor immune responses of CAR-T cells.

### EXAMPLE 11. Combination of Anti-PD-1-TGF-RII with CD19 CAR-T Cells Enhances Tumor Cell Killing

CD19 CAR-T cells are co-cultured with tumor cells expressing CD19 antigen on cell surface in presence of anti-PD-1-TGFRII fusion protein or anti-PD-1. The combination of anti-PD-1-TGFRII fusion protein with CD19 CAR-T cells shows increased killing of tumor cells as compared to the combination of anti-PD-1 and CAR-T cells or CAR-T cells alone.

### EXAMPLE 12. Combination of Anti-PD-1-TGF-RII with ROR1 Specific CAR-T Enhances Tumor Cell Killing

ROR1 CAR-T cells are co-cultured with tumor cells expressing ROR1 antigen on cell surface in presence of anti-PD-1-TGFRII fusion protein or anti-PD-1. The combination of anti-PD-1-TGFRII fusion protein with ROR1 CAR-T cells shows increased killing of tumor cells as compared to the combination of anti-PD-1 and CAR-T cells or CAR-T cells alone.

### EXAMPLE 13. Combination of Anti-PD-1-TGF-RII with BCMA Specific CAR-T Improves Cytotoxic Potential of CAR-T Cells

BCMA specific CAR-T cells are co-cultured with tumor cell line expressing BCMA antigen in presence or absence of anti-PD-1-TGFRII fusion protein or anti-PD-1. The combination of anti-PD-1-TGFRII fusion protein with CAR-T cells improves cytotoxic potential of CAR-T cells compared to the combination of anti-PD-1 and CAR-T cells or CAR-T cells alone.

### EXAMPLE 14. Combination of Anti-PD-1-TGF-RII with PSMA Specific CAR-T Enhances Tumor Cell Killing by CAR-T Cells

PSMA specific CAR-T cells are co-cultured with tumor cell line expressing PSMA antigen in presence or absence of anti-PD-1-TGFRII fusion protein or anti-PD-1. The combination of anti-PD-1-TGFRII fusion protein with CAR-T cells improves cytotoxicity of CAR-T cells when compared to the combination of anti-PD-1 and CAR-T cells or CAR-T cells alone.

### EXAMPLE 15. NK Cell Target Cell Lysis was Improved by Anti-PD-1-TGFRII

The ability of anti-PD-1 (VH7/VL6)-TGFRII and anti-PD-1 (VH6/VL5)-TGFRII to promote natural killer cell-mediated killing of tumor cells was assessed in an in vitro model of ovarian cancer. Effector NK cells were purified from healthy donor and co-cultured with SK-OV-3 target cell line overnight at different E: T cell ratios in the presence of anti-RSV-TFGBRII (control), anti-PD-1 (VH6/VL5) and anti-PD-1 (VH7/VL6), anti-PD-1 (VH7/VL6)-TGFRII and anti-PD-1 (VH6/VL5)-TGFRII. Propidium iodine was added to the culture to identify dead cells and cell death was assayed using the Nexcelom Celigo.

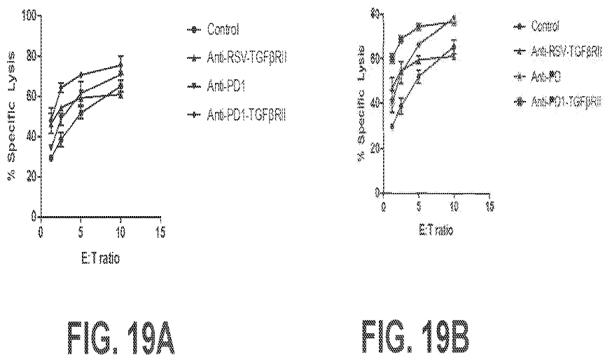

As seen in FIG. 19A and FIG. 19B, anti-PD-1-TGFβRII fusion proteins promoted higher tumor cell killing compared to anti-PD-1 or the TGFβRII alone (Anti-RSV-TGFβRII).

### EXAMPLE 16: Fusion Protein of Anti-PD-1-ADA2

Anti-PD-1 VHs and VLs were synthesized and formatted in IgG, scFv-Fc or scFv configurations with adenosine deaminase 2 (ADA2) fused at C-terminal by linker (G4S)₂ (SEQ ID NO: 563). Anti-PD-1-adenosine deaminase fusion proteins were transiently expressed in Expi293 cells according to the manufacturer's protocol and purified using AKTA AVANT system.

### Surface Plasmon Resonance

Surface plasma resonance (SPR) analysis was performed using Biacore3000, CM5 chip, an amine-coupling kit, 10×HBS-P running buffer and Glycine for. For anti-PD-1 kinetic assay, recombinant PD-1 Fc protein was immobilized on chip using a pre-defined ligand immobilization program. Purified anti-PD-1-adenosine deaminase fusion proteins were diluted in running buffer to a range of final concentrations and injected. Dissociation was allowed to proceed followed by regeneration of the chip surface.

### Table 10. Affinity of Anti-PD-1-ADA2 to Immobilized PD-1

| | K_{D} |
|---|---|
| Fusion protein | (M) |
| Anti-PD-1 (VH7-VL6) IgG4-ADA2 | 6.63e−14 |
| Anti-PD-1 (VH6-VL5) IgG4-ADA2 | 6.16e−14 |
| Anti-PD-1 (VH7-VL6) IgG1-ADA2 | 5.09e−13 |
| Anti-PD-1 (VH6-VL5) IgG1-ADA2 | 3.09e−13 |
| Anti-PD-1 (VH7-VL6) scFv-Fc-ADA2 | 5.63e−10 |
| Anti-PD-1 (VH6-VL5) scFv-Fc-ADA2 | 1.84e−12 |
| Anti-PD-1 (VH7-VL6) scFv-ADA2 | 1.3e−10 |
| Anti-PD-1 (VH6-VL5) scFv-ADA2 | 3.46e−9 |

### PD-1/PD-L1 Blockade Assay

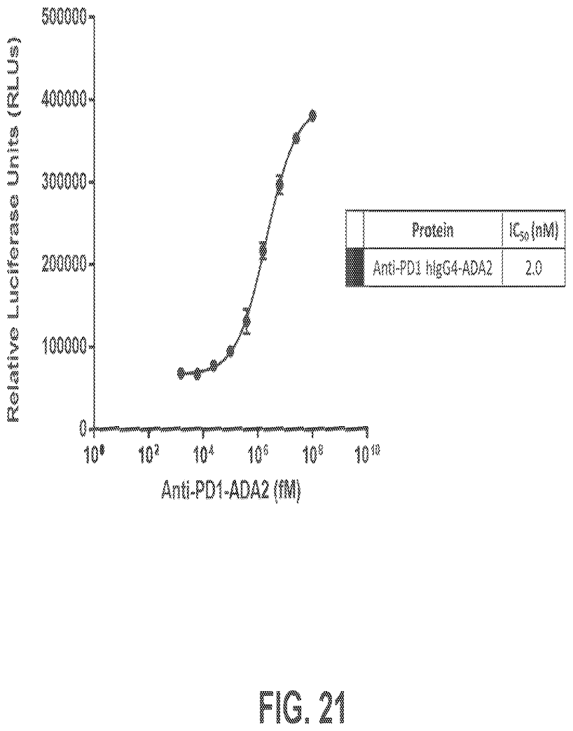

To investigate whether the anti-PD-1-ADA2 fusion proteins could function as a PD-1 inhibitor by blocking the PD-1/PD-L1 interaction, a PD-1/PD-L1 blockade bioassay kit from Promega was used as described in Example 1. The values in Table 10 were used as a measure of PD-1/PD-L1 blocking potency (FIG. 21).

### ADA Enzymatic Activity Assay

To determine the anti-PD-1-ADA2 fusion protein enzymatic activity, the colorimetric Adenosine Deaminase (ADA) Activity Kit (Abcam) was utilized according to the manufacturer's recommendations. Two-minute and 30-minute time points were selected to calculate the ADA enzymatic activity over time.

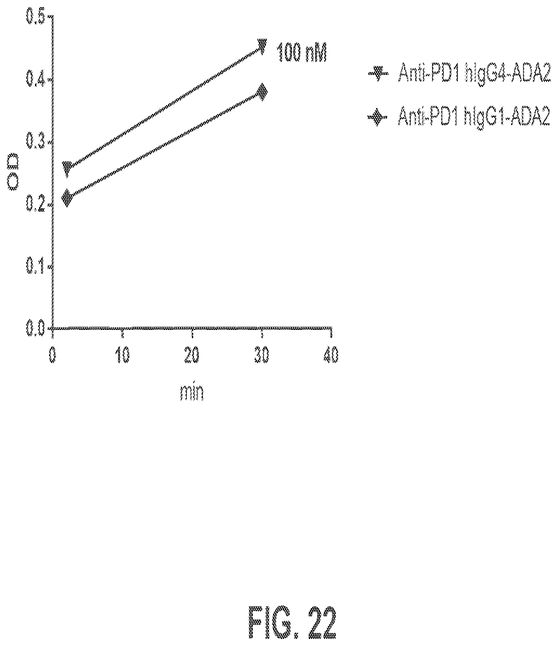

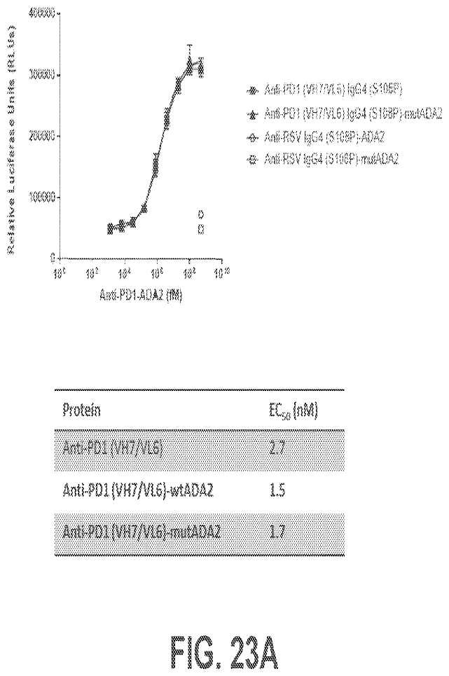

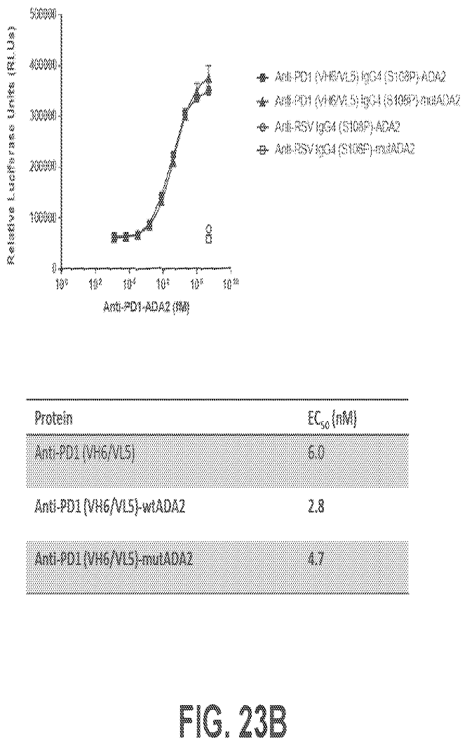

As shown in FIG. 22, anti-PD-1-ADA2 fusion proteins exhibited enzymatic activity over time.

### EXAMPLE 17: PD-1 Binding Affinity Analysis of Anti-PD-1 IgG4 S108P-wtADA2 and -mutADA2

PD-1-Fc antigen and reference were immobilized on a Biacore CM5 surface, then 6-12 replicated series diluted concentrations of anti-PD-1-IgG4-S108P-wtADA2 and -mutADA2 were series-injected on PD-1-Fc antigen and reference surface. Kinetics data was evaluated for 1:1 model: Langmuir with mass transfer. Results are shown in Table 11.

Table 11. Binding Affinity of Anti-PD-1 (VH6/VL5) and (VH7/VL6) IgG4 S108P and the Anti-PD-1-wtADA2 and -mutADA2

| | K_{D} |
|---|---|
| Analyte | (M) |
| Anti-PD-1 (VH6/VL5) IgG4 S108P | 3.34e−13 |
| Anti-PD-1 (VH6/VL5) IgG4 S108P-ADA2 | 7.53e−13 |
| Anti-PD-1 (VH6/VL5) IgG4 S108P-mutADA2 | 2.18e−13 |
| Anti-PD-1 (VH7/VL6) IgG4 S108P | 3.92e−13 |
| Anti-PD-1 (VH7/VL6) IgG4 S108P-ADA2 | 4.13e−13 |
| Anti-PD-1 (VH7/VL6) IgG4 S108P-mutADA2 | 3.38e−13 |

### EXAMPLE 18: Anti-PD-1-ADA2 Fusion Proteins Effectively Blocked PD-L1/PD-1 Signaling

Ability of anti-PD-1-ADA2 fusion proteins to block PD-1/PD-L1 interaction was evaluated using a reporter bioassay as mentioned in Example 12.

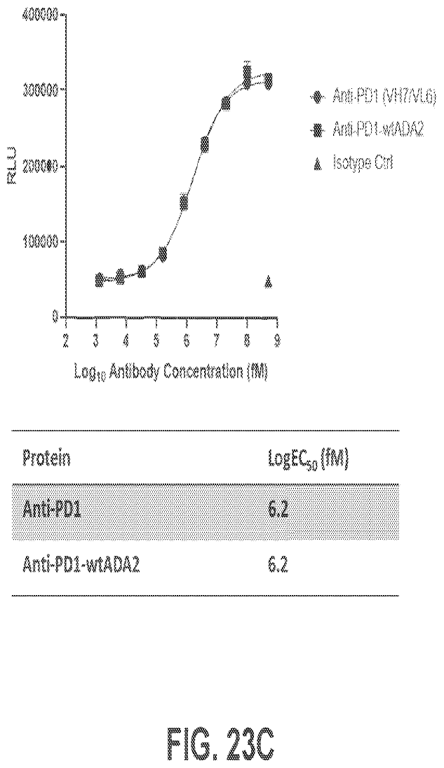

As shown in FIG. 23A-23B, anti-PD-1 (VH7/VL6)-wtADA2 and -mut7ADA2, and anti-PD-1 (VH6/VL5)-wtADA2 and -mut7ADA2 blocked PD-1/PD-L1 interaction with similar potency.

### EXAMPLE 19: ADA2 Enzymatic Activity

The ability of anti-PD-1-ADA2 fusion proteins to enzymatically degrade adenosine was evaluated utilizing an in vitro ADA enzymatic activity assay, as described in Example 12.

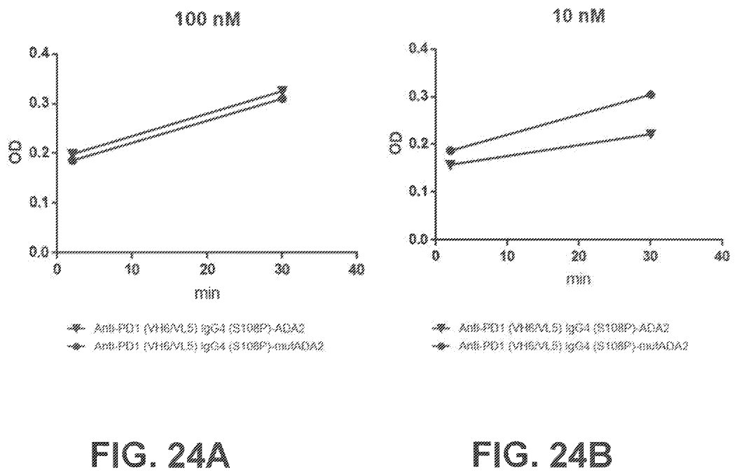

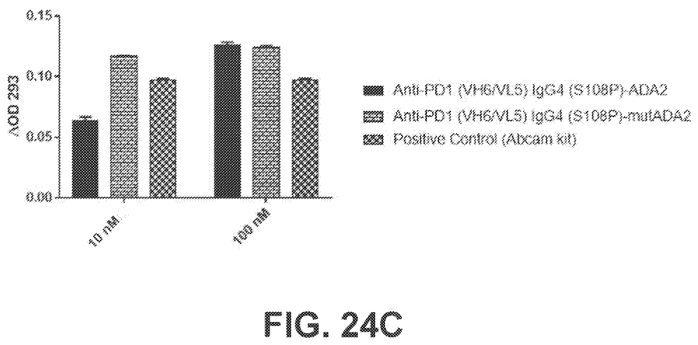

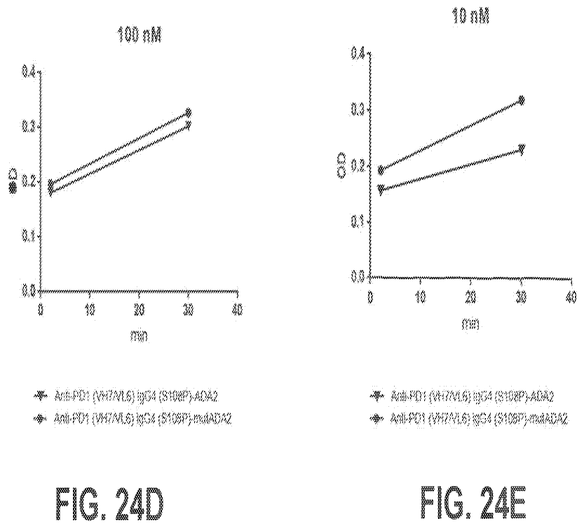

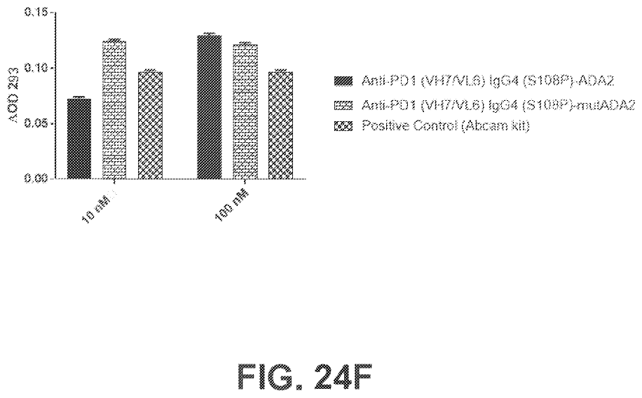

As shown in FIG. 24A-24F, at lower concentrations of adenosine, the mut7ADA2 has higher enzymatic activity as compared to the wtADA2.

### EXAMPLE 20: Anti-PD-1-Mut7-ADA2 has Higher Enzymatic Activity than Anti-PD-1-wtADA2

The Michaelis Menten constants (Km) of anti-PD-1 (VH6/VL5)-wtADA2 and anti-PD-1 (VH6/VL5)-mut7ADA2 were measured using the fluorometric ADA2 enzyme assay kit.

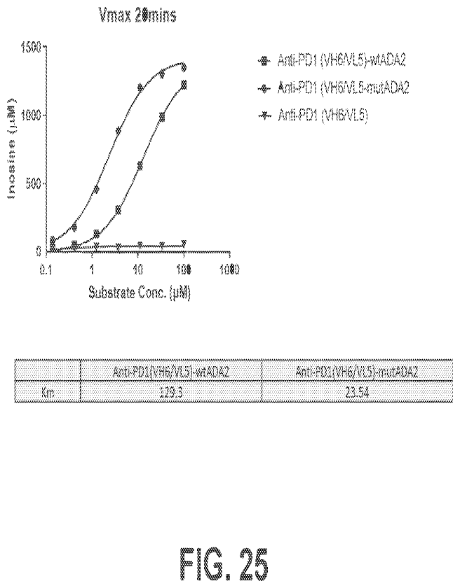

As shown in FIG. 25, anti-PD-1-mut7ADA2 has a higher Km compared to anti-PD-1-wtADA2.

### EXAMPLE 21: Anti-PD-1-wtADA2 Reverses Adenosine-Mediated Suppression of T Cell Proliferation

The ability of anti-PD-1-wtADA2 to promote T cell proliferation was assessed in vitro using normal donor PBMCs. CellTrace Violet-labeled PBMCs co-cultured in media containing 1 mM adenosine were stimulated in the presence of increasing concentrations of anti-PD-1 (VH6/VL5)-wtADA2 and anti-PD-1 (VH6/VL5). The supernatants were collected for cytokine analysis. The cells were assayed for T cell proliferation by flow cytometry.

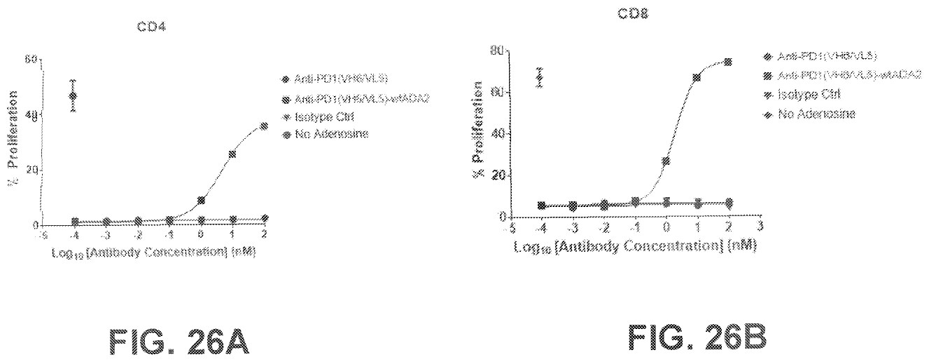

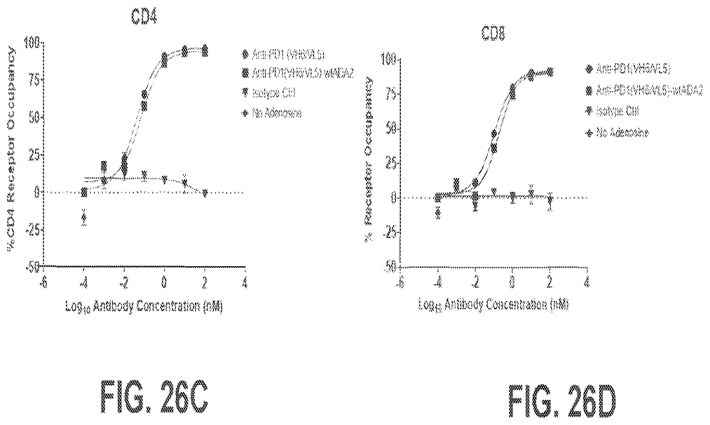

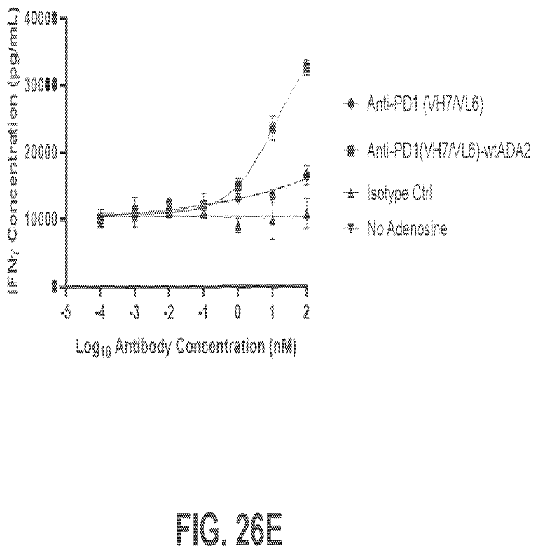

The data showed that anti-PD-1 (VH6/VL5)-wtADA2 significantly reversed the adenosine-mediated suppression of CD4+ (FIG. 26A) and CD8+ T cells (FIG. 26B) as compared to anti-PD-1 (VH6/VL5) or isotype control. As shown in FIGS. 26C and D, both anti-PD-1 and anti-PD-1-wtADA2 similarly occupied PD-1 receptor on CD4+ (FIG. 26C) and CD8+ T cells (FIG. 26D).

The ability of anti-PD-1 (VH7/VL6)-wtADA2 to promote T cells function was further assessed by stimulating PBMCs in media containing adenosine in the presence of increasing concentrations of anti-PD-1 (VH7/VL6), anti-PD-1 (VH7/VL6)-wtADA2, or isotype controls. The culture supernatants were collected and assayed for IFNγ production.

FIG. 26E shows that anti-PD-1 (V_{H}7/V_{L}6)-wtADA2 treatment induced significantly more IFN-γ as compared to anti-PD-1 or isotype control.

### EXAMPLE 22: WtADA2 and mutADA2 Showed Comparable Reversal of Adenosine-Mediated Suppression of T Cell Proliferation

The effectiveness of wtADA2 and mutADA2 to reverse adenosine-mediated suppression of T cell proliferation was assessed by stimulating labeled PBMCs in a media containing adenosine in the presence of increasing concentration of anti-PD-1-wtADA2, anti-PD-1-mutADA2, anti-RSV-wtADA2, and anti-RSV-mutADA2. The cells were stained, then analyzed for proliferation by flow cytometry.

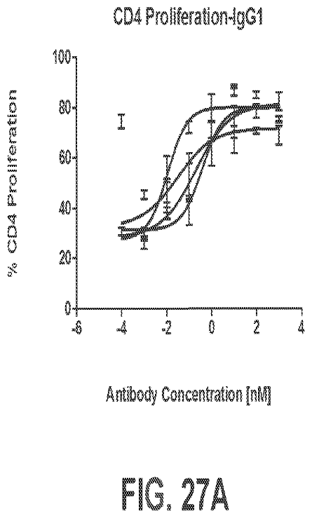

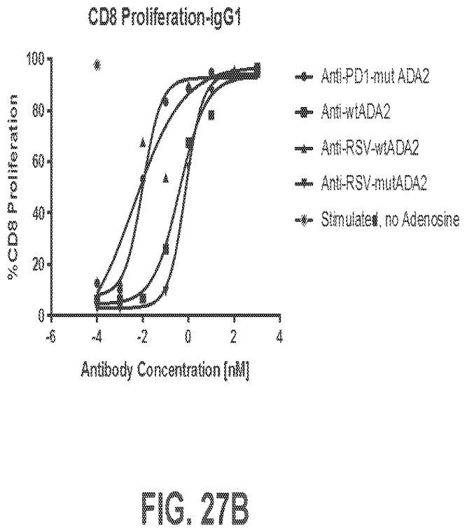

As shown in FIG. 27, both the wtADA2 and mutADA fusion proteins were equally effective at reversing the adenosine-mediated suppression of CD4 T cells (FIG. 27A) and CD8 T cells (FIG. 27B).

### EXAMPLE 23: Blockade of PD-1-PDL1 Interaction by Anti-PD-1 (VH6/VL5)-ADA2

The ability of various forms of anti-PD-1-ADA2 fusion proteins to block PD-1/PD-L1 interaction was evaluated using a reporter bioassay as mentioned in Example 12.

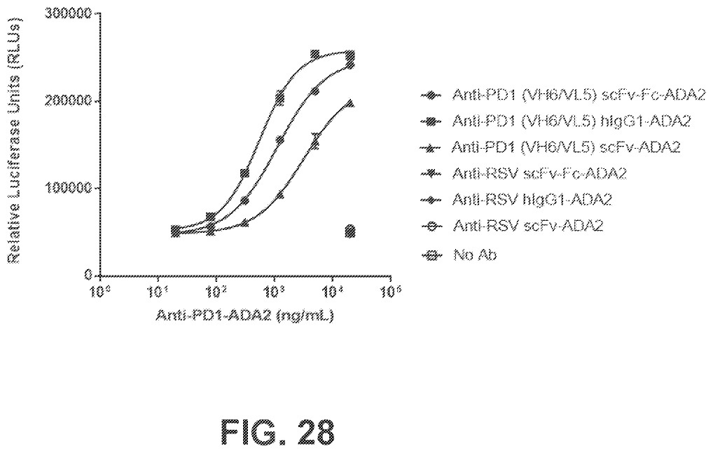

As shown in FIG. 28, anti-PD-1 (VH6/VL5) scFv-Fc-ADA2, hlgG1-ADA2, and scFv-ADA2 all showed functional activity as measured by the PD-1/PD-L1 Blockade Bioassay.

### EXAMPLE 24: ADA Enzymatic Activity of Anti-PD-1-ADA2-scFv-Fc

The ability of various forms of anti-PD-1-ADA2 fusion proteins to enzymatically degrade adenosine was evaluated in an in vitro ADA enzymatic activity assay, as described in Example 12.

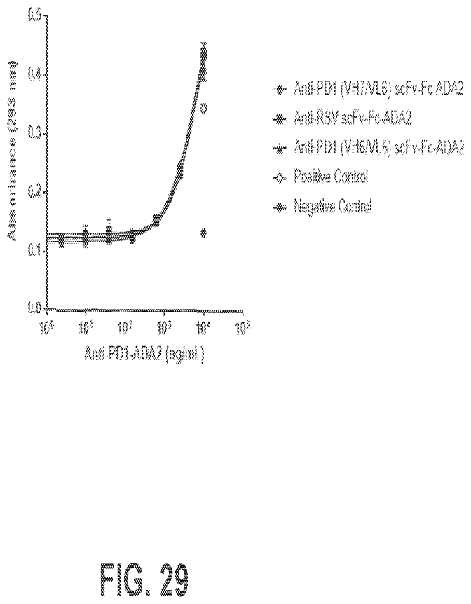

As shown in FIG. 29, anti-PD-1 (VH7/VL6) scFv-Fc-ADA2 and (VH6/VL5) scFv-Fc-ADA2 had similar ADA2 enzymatic activity as compared to the control.

### EXAMPLE 25: ADA Enzymatic Activity of Various Anti-PD-1-ADA2 and Anti-PD-1-mutADA2 Constructs

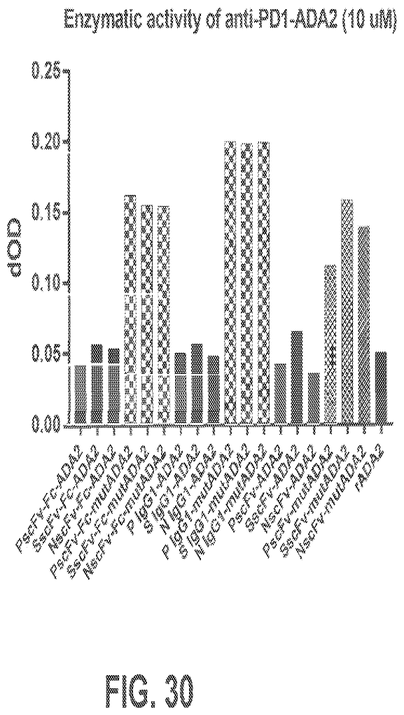

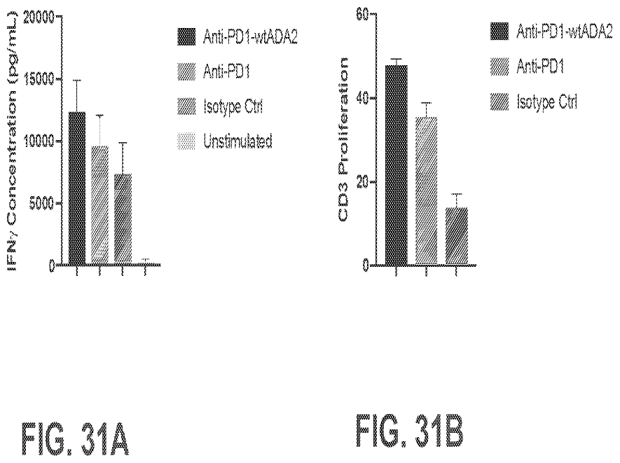

ADA enzymatic activity assay was conducted as described in Example 12. As shown in FIG. 30, at 10 nM of adenosine, mutADA2 was seen to exhibit higher enzymatic activity as compared to the wtADA2.

### EXAMPLE 26: Anti-PD-1-ADA2 Promoted IFN-γ Production and Proliferation of Tumor-Infiltrating Lymphocytes (TILs) in Primary CRC Patient Tumor

PBMCs purified from CRC patients were co-cultured with matched dissociated tumor cells. CellTrace Violet-labeled cells were stimulated with anti-CD3 and anti-CD28 in the presence of isotype control, anti-PD-1 (VH6/VL5), or anti-PD-1 (VH6/VL5)-wtADA2 fusion protein. The culture supernatants were collected and IFN-γ levels were quantified by MSD per the manufacturer's protocol and cells were assessed for T cell proliferation. For gene expression analysis, the cells pellets were harvested by centrifugation and RNA was purified using the Qiagen RNAeasy micro kit according the manufacturer's protocol. Purified RNA was used for gene expression analysis using the nanostring according to the manufacturer's instructions. Gene expression was analyzed using Nanostring nCounter software.

As shown in FIG. 31A, anti-PD-1-wtADA2 fusion protein promoted higher level of IFN-γ production and T cell proliferation (FIG. 31B) as compared to anti-PD-1 or isotype control. In addition, anti-PD-1-wtADA2 fusion protein treatment resulted in significant upregulation of IFN-γ signaling and chemokine signaling genes and cytotoxic genes (FIG. 31C) as compared to anti-PD-1 treatment, highlighting improved cytotoxic function of T cells in the presence of tumor as compared to an anti-PD-1 antibody.

### EXAMPLE 27: Effect of Anti-PD-1-wtADA2 Fusion Protein in a Humanized Mouse Model of Lung Cancer

NSG mice humanized with PBMCs were inoculated intra-prancreatically (ortotopic injection) with the pancreatic cancer cell line Panc.08.fLuc.cGFP. Mice body weight was measured weekly and tumor weights were measured at the end of the study on day 38. Mice were randomized into treatment groups and treated twice weekly with HBSS (vehicle control), anti-PD-1 (VH6/VL5), and anti-PD-1 (VH6/VL5)-wtADA2.

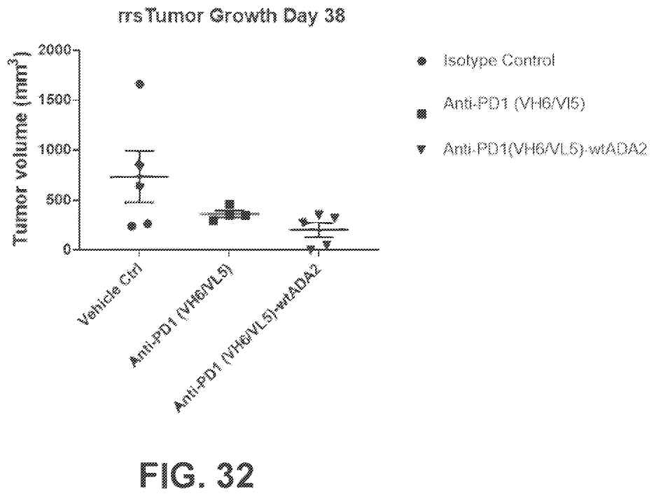

As can be seen in FIG. 32, mice treated with anti-PD-wtADA2 had significantly smaller tumors compared to mice treated with anti-PD-1 or isotype control.

### EXAMPLE 28: Treatment of Anti-PD-1 (V_{H}6/V_{L}5)-wtADA2 Downregulated the Adenosine Pathway, Angiogenesis and Upregulated Cytotoxicity and Cytokine Genes

PBMCs purified from CRC patients were co-cultured with matched dissociated tumor cells. CellTrace Violet-labeled cells were stimulated with anti-CD3 and anti-CD28 in the presence of isotype control, anti-PD-1 (VH6/VL5) or anti-PD-1 (VH6/VL5)-wtADA2 fusion protein. The culture supernatants were collected and IFN-γ levels were quantified by MSD per manufacturer's protocol and cells assess for T cell proliferation. For gene expression analysis, the cultules were duplicated and the cells pellets were harvested by centrifugation and RNA was purified using the Qiagen RNAeasy micro kit according the manufacturer's protocol. Purified RNA was used for gene expression analysis using the nanostring according to the manufacturer's instructions. Gene expression was analyzed using Nanostring nCounter software.

Anti-PD-1-wtADA2 fusion protein promoted higher level of IFN-γ production and T cell proliferation compared to anti-PD-1 or isotype control (data not shown). Anti-PD-1-wtADA2 fusion protein treatment also resulted in significant upregulation of IFN-γ signaling and chemokine signaling genes and cytotoxic genes compared to anti-PD-1 treatment highlighting improved cytotoxic function of T cells in the presence of tumor compared to an anti-PD-1 antibody (data not shown).

### EXAMPLE 29: Anti-PD-1 (VH7-VL6)-mutADA2 Inhibits the PDL-1/PD1 Pathway

To assess the effect of an anti-PD-1 (V_{H}7-V_{L}6)-mutADA2 on the PDL1/PD1 pathway, the Promega PD1/PDL1 bloackade assay was used. Briefly, effector Jurkat T cells expressing PD-1 and a luciferase reporter under a Nuclear Factor of Activated T-Cells (“NFAT”) response element, artificial antigen presenting cells (aAPC), and CHO-K1 cells engineered to express PD-L1 and a T cell receptor activator were thawed and rested overnight in complete T cell media at 37° C. in a CO₂ incubator. The cells were then co-cultured in the presence of increasing concentrations of anti-PD-1 (VH7-V_{L}6)-mutADA2. The bioluminescent signal was detected and quantified using the Bio-Glo™ Luciferase Assay System and the GloMax® Discover System luminometer.

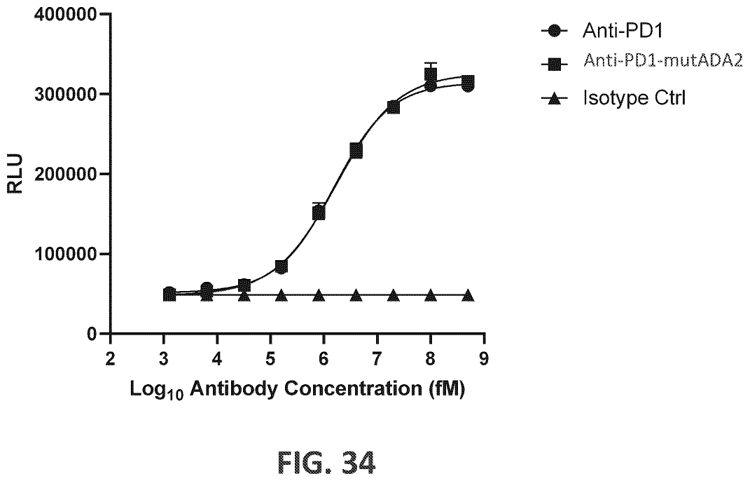

The addition of anti-PD-1 (VH7-VL6)-mutADA2 was found to inhibit the inhibitory signal and reverse the bioluminescence signal. As can been seen in FIG. 34, anti-PD-1 (VH7/VL6)-mutADA2 inhibits the the inhibitory signal of PD1/PDL1 pathway in a dose-dependent manner.

### EXAMPLE 30: Anti-PD-1 (V_{H}6/V_{L}5)-wtADA2 Catalyzes Adenosine into Inosine in Dose-Dependent and Substrate Concentration-Dependent Manner

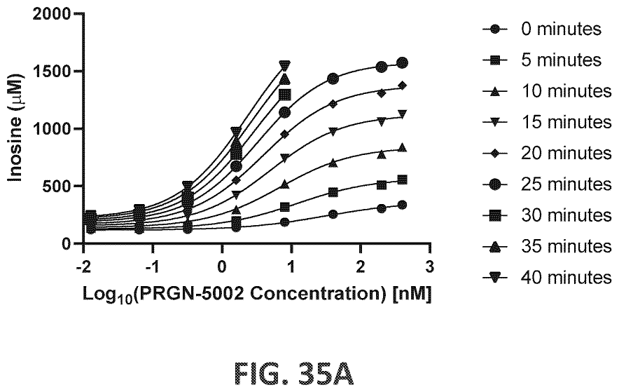

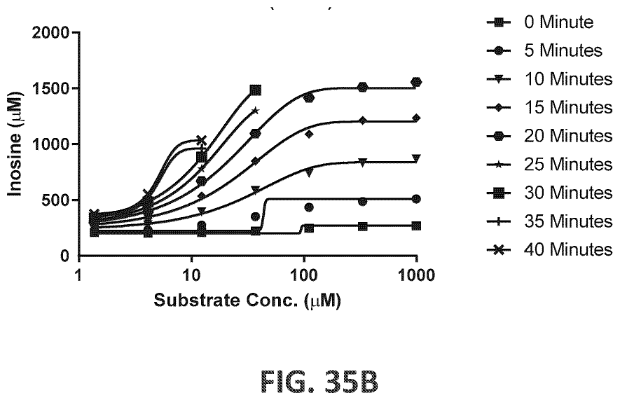

To assess the ability of anti-PD-1 (VH6/VL6)-wtADA2 to catalyze adenosine into inosine, the adenosine deaminase activity assay was used. Initially 100 μM of adenosine was incubated with different concentrations of anti-PD-1 (VH6/VL6)-wtADA2 at different times. As can been seen FIGS. 35A-35B, anti-PD-1 (VH6/VL6)-wtADA2 catalyzed adenosine in both a concentration and time dependent manner.

### EXAMPLE 31: Anti-PD-1 (VH6/VL5)-wtADA2 and Anti-PD-1 (VH6/VL5)-mutADA2 Reverses Adenosine-Mediated Suppression of T Cell Proliferation in Dose-Dependent Manner

Adenosine has been shown to suppress T cell activation and effector function (e.g., proliferation, cytokine production, and cytotoxicity). To assess the ability of anti-PD-1 (VH6/VL5)-wtADA2 and anti-PD-1 (VH6/VL5)-mutADA2 to reverse the adenosine-mediated suppression of T cell functions, peripheral mononuclear cells (PBMCs) were stimulated with anti-CD3 antibody in the presence of adenosine (100 mM) and increasing concentrations of either 100 mM, a control antibody-ADA2, or anti-PD1. The PBMCs were stained with CellTrace Violet to monitor T cell proliferation and the culture supernatants were collected at the end of the incubation and screened for IFN-γ production.

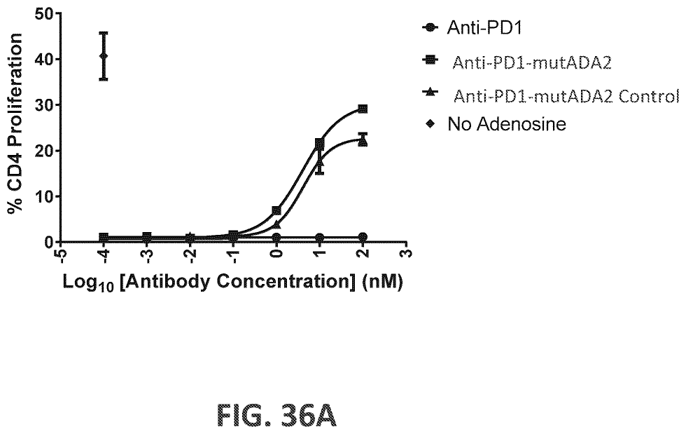

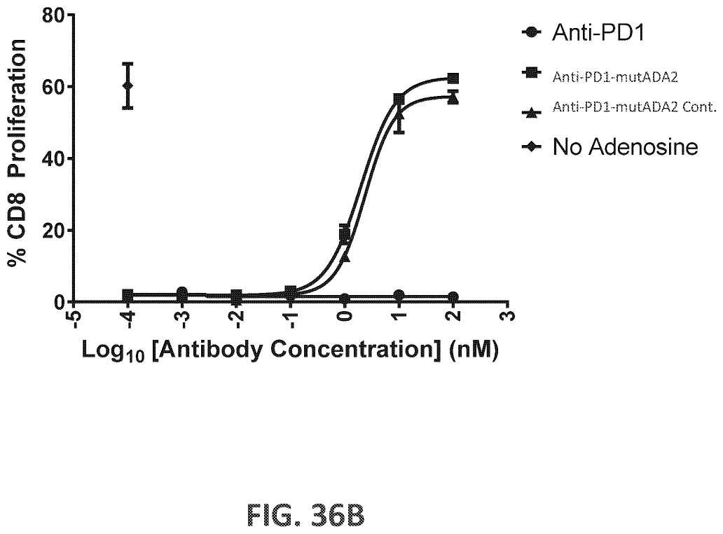

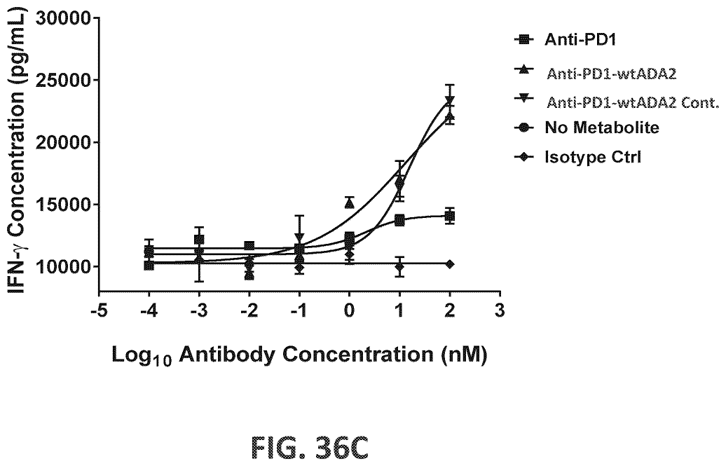

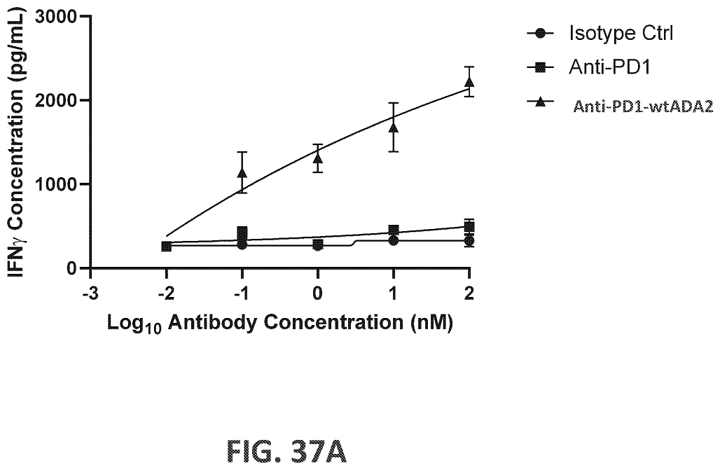

As can be in FIGS. 36A-36C, addition of adenosine was found to completely inhibit T cell proliferation, yet this inhibition was specifically reversed by anti-PD-1 (VH6/VL5)-mutADA2 (FIGS. 36A and 36AB) anti-PD-1 (VH6/VL5)-wtADA2 (FIG. 36C) or its control and not by anti-PDI in a dose-dependent manner.

### EXAMPLE 32: Anti-PD-1 (VH7/VL6)-wtADA2 and Anti-PD-1 (VH6/VL5)-wtADA2 Reverses Inhibition of T Cell Function Mediated by Adenosine Produced by Tumor Cells in the Presence of ATP

To determine whether anti-PD-1 (VH7/VL6)-wtADA2 and anti-PD-1 (VH6/VL5)-wtADA2 is able to reverse the inhibition of T cells function mediated by tumor cells in the presence of ATP, several human tumor cell lines shown to convert ATP into adenosine were co-cultured with the T cells in the presence of ATP and increasing concentrations of either anti-PD-1 (VH7/VL6)-wtADA2 or anti-PD-1 (VH6/VL5)-wtADA2, anti-PD1, or isotype controls. The culture supernatants were collected at the end of the incubation and screened for IFN-γ production.

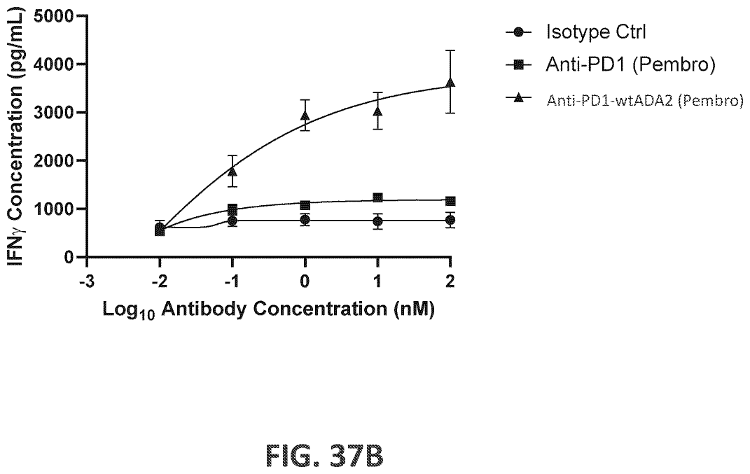

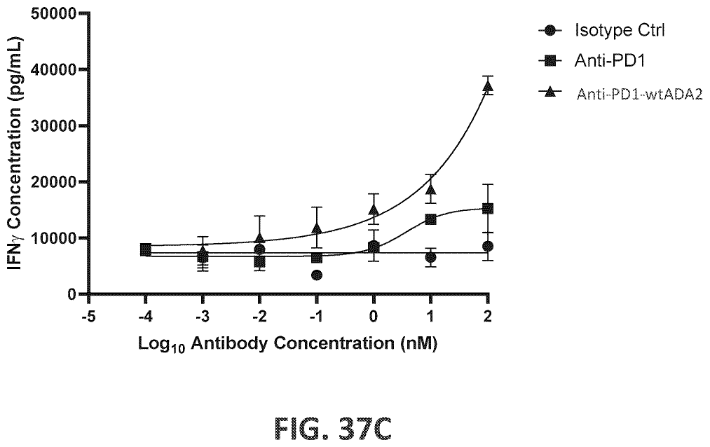

As can been seen in FIGS. 37A and 37B, anti-PD-1 (VH7/VL6)-wtADA2) reversed the suppression of IFN-γ production in a dose-dependent manner in tumor cell lines A549, and SKOV3 respectively. And as can be seen in FIG. 37C, anti-PD-1 (VH6/VL5)-wtADA2 and reversed the suppression of IFN-γ production in a dose-dependent manner in tumor cell line Panc.08.

### EXAMPLE 33: Anti-PD-1 (VH6/VL5)-wtADA2 Inhibits Growth of A549 Tumor in NSG Mouse

To assess the ability of anti-PD-1 (VH6/VL5)-wtADA2 to inhibit or delay tumor growth, A549 tumor cells were implanted subcutaneously into humanized NSG mice. Mice were randomized by tumor volume into treatment groups and administered anti-PD-1 (VH6/VL5)-wtADA2, anti-PD1, or isotype control.

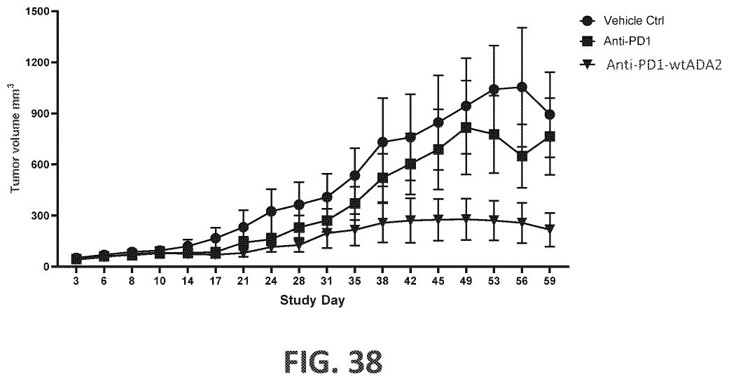

As can be seen in in FIG. 38, anti-PD-1 (VH6/VL5)-wtADA2 significantly delayed A549 tumor growth as compared to anti-PD1 or isotype control.

### EXAMPLE 34: Method of Manufacturing Anti-PD-1 Fusion Proteins

Fusion proteins are synthesized comprising: a) an anti-PD-1 heavy chain variable region comprising the amino acid sequence of any one of SEQ ID NOs: 333-337 or SEQ ID NOS: 384, or SEQ ID NO: 386; and b) an anti-PD-1 light chain variable region comprising the amino acid sequence of any one of SEQ ID NOs: 338-343, SEQ ID NO: 385, or SEQ ID NO: 387. The fusion proteins are formatted in IgG, scFv-Fc or scFv configurations with TGFβRII fused at the C-terminal by linker (G4S)₂ (SEQ ID NO: 563). The anti-PD-1-trap fusion protcins are then transiently expressed in Expi293 cells according to the manufacturer's protocol. To purify the fusion proteins, the transfected supernatants are loaded onto a protein L column using AKTA AVANT and the column is washed with PBS before the protein is eluted with IgG elution buffer (Pierce). The eluted protein is then buffer-exchanged into PBS using a PD-10 column (GE Healthcare).

### EXAMPLE 35: Anti-MUC16 Antibodies

The MUC16 binding affinity of anti-MUC16 IgGs was analyzed using Biacore. MUC16-Fc antigen and reference were immobilized on Biacore CMS surface, 6-12 replicate series diluted concentrations of anti-MUC16 IgG on MUC16-Fc antigen and reference surface were injected. The kinetics were evaluated for 1:1 model: Langmuir with mass transfer.

As shown in Table 12, anti-MUC16 VL10/VHI0, VL7/VH10, VL9/VH10 and VL8/VH2 IgG showed strong interaction with MUC16-Fc antigen by Biacore. The binding affinity (KD) was in the 1-4 pM range.

### Table 12: Biacore Binding Affinity of Humanized Variants of Anti-MUC16 Antibodies

| | | kₐ | k_{d} | K_{D} |
|---|---|---|---|---|
| Ligand | Analyte | (1/Ms) | (1/s) | (M) |
| MUC16- | Anti-MUC16 V_{L}8/V_{H}13 IgG | 3.56e5 | 3.77e−5 | 1.06e−10 |
| Fc | Anti-MUC16 V_{L}10/V_{H}10 IgG | 4.78e5 | 7.42e−7 | 1.55e−12 |
| | Anti-MUC16 V_{L}8/V_{H}10 IgG | 4.62e5 | 2.2e−6 | 4.77e−12 |
| | Anti-MUC16 V_{L}9/V_{H}10 IgG | 6.5e5 | 8.62e−7 | 1.33e−12 |
| | Anti-MUC16 V_{L}8/V_{H}2 IgG | 5.42e5 | 7.31e−7 | 1.35e−12 |

### EXAMPLE 36: TGFβ1 Binding to Anti-MUC16 VL10/VH10 hIgG1-TGFβRII

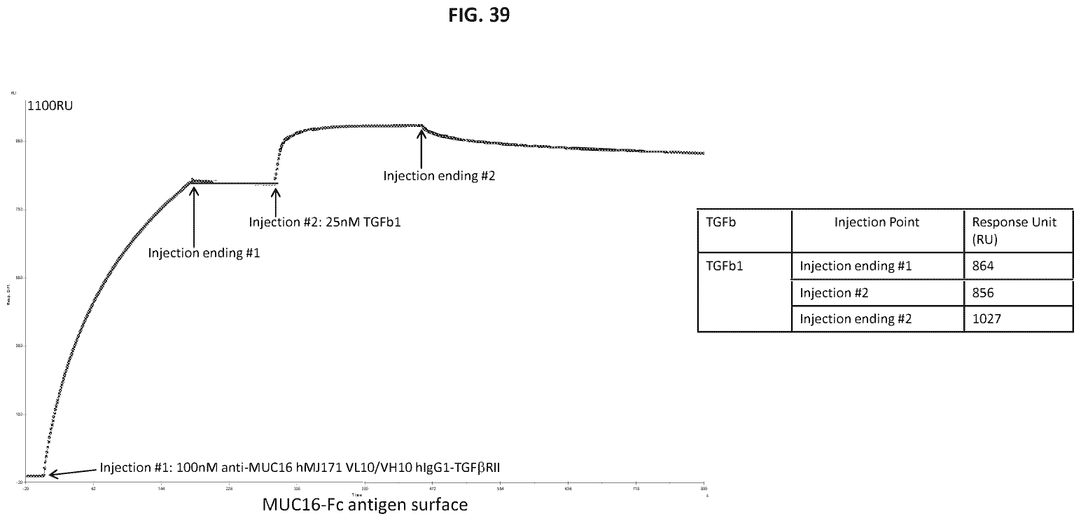

Simultaneous binding of TGFβ1 and MUC16 to anti-MUC16 VL10/VH10 hIgG1-TGFβRII fusion protein was analyzed using Biocore assay. MUC16-Fc antigen was immobilized on Biacore CM5 chip surface. 100 nM anti-MUC16 VL10/VH10 hIgG1-TGFβRII was injected over sensor surfaces followed by the injection of 25 nM TGFβ1 over sensor surfaces. As observed in FIG. 39, anti-MUC16 VLI0/VHI0-TGF-Rllbound to both of MUC16-Fc antigen and TGF-1 using Biacore simultaneous binding assay.

### EXAMPLE 37: Inhibition of TGFβ1 Induced Reporter Gene Activity by Anti-MUC16 VL10/VH10 hIgG1-TGFβRII

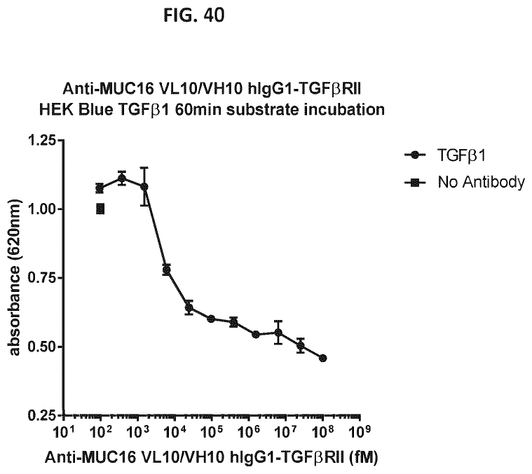

TGFβ1 was diluted and added to a flat-bottom 96-well plate. Anti-MUC16 VL10/VH10 hIgG1-TGFβRII was diluted to 100 nM and serially diluted four-fold. Trap proteins was added to TGF-β and allowed to incubate. HEK Blue TGF-β1 cells were diluted to 0.7e6 cells/mL and added to the plate following incubation. Following the second incubation, supernatant was removed and added to the Quanti-Blue substrate and the plate will be read on the SpectraMax instrument (620 to 655 nm). As shown in FIG. 40, anti-MUC16 VL10/VH10 hIgG1-TGFβRII showed binding to TGFβ1 with IC₅₀-3.8 pM using HEK Blue TGF-β assay.

### Sequences

Provided herein are tables providing a representative list of certain sequences included in the embodiments described herein.

Table 13. Non-limiting exemplary polypeptide and nucleotide sequences

| | SEQ | Amino Acid | SEQ | |
|---|---|---|---|---|
| Name | ID NO | Sequence | ID NO | NUCLEOTIDE SEQUENCE |
| Anti- | 1 | QVQLVESGGGVVQP | | |
| PD-1 | | GRSLRLSCAASGFT | | |
| VH1 | | FRSYGMHWVRQAPG | | |
| | | KGLEWVAIIFYDGS | | |
| | | NKYYADSVKGRFTI | | |
| | | SRDNSKNTLYLQMN | | |
| | | SLRAEDTAVYYCAR | | |
| | | DDDYWGQGALVTVS | | |
| | | S | | |
| Anti- | 2 | EVQLVQSGGGVVQP | | |
| PD-1 | | GRSLRLSCAASGFT | | |
| VH2 | | FSSYGMHWVRQAPG | | |
| | | KGLEWVAVIWYDGS | | |
| | | NKYYADSVKGRFTI | | |
| | | SRDNSKNTLYLQMN | | |
| | | SLRAEDTAVYYCAG | | |
| | | EGFDYWGQGTLVTV | | |
| | | SS | | |
| Anti- | 3 | QVQLVQSGAEVKKP | | |
| PD-1 | | GASVKVSCKASGYT | | |
| VH3 | | FTGYYMHWVRQAPG | | |
| | | QGLEWMGAINPNSG | | |
| | | GTNYAQKFQGRVTM | | |
| | | TRDTSISTAYMELS | | |
| | | RLRSDDTAVYYCAR | | |
| | | RPDRANWHFDYWGQ | | |
| | | GTLVTVSS | | |
| Anti- | 4 | QVQLVQSGAEVKKP | | |
| PD-1 | | GASVKVSCKASGYT | | |
| VH4 | | FTGYYMHWVRQAPG | | |
| | | QGLEWMGAINPNSG | | |
| | | GTNYAQKLQGRVTM | | |
| | | TTDTSTSTAYMELR | | |
| | | SLRSDDTAVYYCAR | | |
| | | HGLKGDGYFDYWGQ | | |
| | | GTLVTVSS | | |
| Anti- PD-1 VH5 | 5 | 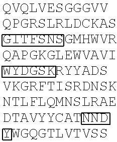 | | |
| Anti- | 6 | QVQLVESGGGVVQP | 109 | CAGGTGCAGCTGGTCGAAAGCGGAGGA |
| PD-1 | | GRSLRLDCKASGIT | | GGAGTGGTCCAGCCAGGACGATCCCTG |
| VH6 | | FSNSGMHWVRAQPG | | AGACTGGATTGTAAGGCCTCTGGAATC |
| | | KGLEWVAVIWYDGS | | ACATTCTCTAACAGTGGAATGCACTGG |
| | | KRYYADSVKGRFTI | | GTGCGCCAGGCACCAGGAAAAGGACTG |
| | | SRDNSKNTLFLQMN | | GAGTGGGTGGCCGTCATCTGGTACGAC |
| | | SLRAEDTAVYYCAT | | GGGTCAAAGCGATACTATGCAGATAGC |
| | | NDDYWGQGTLVTVS | | GTGAAAGGAAGGTTCACAATTTCACGC |
| | | S | | GACAACAGCAAGAATACTCTGTTTCTG |
| | | | | CAGATGAACTCTCTGAGAGCAGAGGAT |
| | | | | ACTGCCGTGTACTATTGTGCTACCAAT |
| | | | | GACGATTATTGGGGGCAGGGAACTCTG |
| | | | | GTGACCGTCAGTTCA |
| Anti- | 7 | QVQLVQSGVEVKKP | 110 | CAGGTGCAGCTGGTCCAGAGCGGCGTG |
| PD-1 | | GASVKVSCKASGYT | | GAAGTCAAGAAACCCGGGGCCTCAGTG |
| VH7 | | FTNYYMYWVRQAPG | | AAGGTCAGCTGTAAAGCTTCCGGCTAC |
| | | QGLEWMGGINPSNG | | ACCTTCACAAACTACTATATGTATTGG |
| | | GTNFNEKFKNRVTL | | GTGAGACAGGCACCAGGACAGGGACTG |
| | | TTDSSTTTAYMELK | | GAGTGGATGGGCGGGATTAACCCTAGT |
| | | SLQFDDTAVYYCAR | | AATGGAGGCACTAACTTCAACGAAAAG |
| | | RDYRFDMGFDYWGQ | | TTTAAAAACAGGGTGACCCTGACCACA |
| | | GTTVTVSS | | GATTCAAGCACTACCACAGCTTACATG |
| | | | | GAGCTGAAGTCCCTGCAGTTTGACGAT |
| | | | | ACAGCCGTGTACTATTGTGCTCGGAGA |
| | | | | GACTACAGGTTCGATATGGGCTTTGAC |
| | | | | TATTGGGGCCAGGGGACTACCGTGACC |
| | | | | GTCTCCTCT |
| Anti- | 8 | EIVMTQSPATLSLS | | |
| PD-1 | | PGERATLSCRASQS | | |
| VL1 | | VSSYLAWYQQKPGQ | | |
| | | APRLLIYDASNRAT | | |
| | | GIPARFSGSGSGTD | | |
| | | FTLTISSLEPEDFA | | |
| | | VYYCQQYNNWPRTF | | |
| | | GQGTKVEIK | | |
| Anti- | 9 | EIVLTQSPATLSLS | | |
| PD-1 | | PGERATLSCRASQS | | |
| VL2 | | VSSYLAWYQQKPGQ | | |
| | | APRLLIYDASNRAT | | |
| | | GIPARFSGSGSGTD | | |
| | | FTLTISSLEPEDFA | | |
| | | VYYCQQRSNWPKTF | | |
| | | GQGTKVEIK | | |
| Anti- | 10 | RNVLTQSPLSLPVT | | |
| PD-1 | | PGEPASISCRSSQS | | |
| VL3 | | LSSSGYTYLDWYLQ | | |
| | | KPGQSPQLLIYLAS | | |
| | | WRDSGVPDRFSGSG | | |
| | | SGTDFTLKISRVEA | | |
| | | EDVGVYYCMQAEQT | | |
| | | PGPGNTFGQGTKLE | | |
| | | IK | | |
| Anti- | 11 | DVVMTQSPLSLPVT | | |
| PD-1 | | PGEPASISCRSSQS | | |
| VL4 | | LLHTNGYNYLHWYL | | |
| | | QKPGQSPQLLIYLG | | |
| | | SWQDSGVPDRFSGS | | |
| | | GSGTDFTLKISRVE | | |
| | | AEDVGVYYCMQAEQ | | |
| | | TPRTFGQGTRLEVK | | |
| Ant- | 12 | EIVLTQSPATLSIS | 111 | GAGATCGTCCTGACACAGAGTCCAGCA |
| PD-1 | | PGERATLSCRASQS | | ACTCTGAGCCTGTCCCCCGGCGAACGA |
| VL5 | | VSSYLAWYQQKPGQ | | GCTACTCTGTCCTGCCGGGCATCTCAG |
| | | APRLLIYDASNRAT | | AGTGTGTCTAGTTACCTGGCCTGGTAT |
| | | GIPARFSGSGSGTD | | CAGCAGAAGCCCGGCCAGGCTCCTAGG |
| | | FTLTISSLEPEDFA | | CTGCTGATCTACGACGCCAGCAACAGA |
| | | VYYCQQSSNWPRTF | | GCTACCGGGATTCCTGCCAGGTTCTCA |
| | | GQGTKVEIK | | GGCAGCGGGTCCGGAACAGACTTTACC |
| | | | | CTGACAATCTCAAGCCTGGAGCCCGAA |
| | | | | GATTTCGCTGTGTACTATTGCCAGCAG |
| | | | | TCCTCTAATTGGCCTCGCACCTTTGGC |
| | | | | CAGGGGACAAAGGTCGAGATCAAG |
| Anti- | 13 | EIVLTQSPATLSLS | 112 | GAGATCGTCCTGACTCAGTCCCCAGCA |
| PD-1 | | PGERATLSCRASKG | | ACCCTGAGTCTGTCACCAGGAGAAAGG |
| VL6 | | VSTSGYSYLHWYQQ | | GCAACCCTGAGCTGCCGAGCATCCAAG |
| | | KPGQAPRLLIYLAS | | GGGGTGAGCACATCCGGATACTCTTAT |
| | | YLESGVPARFSGSG | | CTGCACTGGTACCAGCAGAAACCCGGA |
| | | SGTDFTLTISSLEP | | CAGGCTCCTCGACTGCTGATCTACCTG |
| | | EDFAVYYCQHSRDL | | GCATCTTATCTGGAGAGTGGCGTGCCT |
| | | PLTFGGGTKVEIK | | GCTCGGTTCTCTGGGAGTGGATCAGGC |
| | | | | ACCGATTTTACACTGACTATTTCTAGT |
| | | | | CTGGAGCCAGAAGATTTCGCAGTGTAC |
| | | | | TATTGCCAGCATTCTCGAGACCTGCCC |
| | | | | CTGACATTTGGCGGGGGAACTAAGGTC |
| | | | | GAGATCAAA |
| TGFβRII | 14 | IPPHVQKSVNNDMI | | |
| ECD | | VTDNNGAVKFPQLC | | |
| | | KFCDVRFSTCDNQK | | |
| | | SCMSNCSITSICEK | | |
| | | PQEVCVAVWRKNDE | | |
| | | NITLETVCHDPKLP | | |
| | | YHDFILEDAASPKC | | |
| | | IMKEKKKPGITFFM | | |
| | | CSCSSDECNDNIIF | | |
| | | SEEYNTSNPD | | |
| TGFβRII2 | 141 | IPPHVQKSDVEMEA | | |
| ECD | | QKDEIICPSCNRTA | | |
| | | HPLRHINNDMIVTD | | |
| | | NNGAVKFPQLCKFC | | |
| | | DVRFSTCDNQKSCM | | |
| | | SNCSITSICEKPQE | | |
| | | VCVAVWRKNDENIT | | |
| | | LETVCHDPKLPYHD | | |
| | | FILEDAASPKCIMK | | |
| | | EKKKPGETFFMCSC | | |
| | | SSDECNDNIIFSEE | | |
| | | YNTSNPD | | |
| TGFβRII | 142 | PQLCKFCDVRFSTC | | |
| min | | DNQKSCMSNCSITS | | |
| ECD | | ICEKPQEVCVAVWR | | |
| | | KNDENITLETVCHD | | |
| | | PKLPYHDFILEDAA | | |
| | | SPKCIMKEKKKPGE | | |
| | | TFFMCSCSSDECND | | |
| | | NIIFSEEYNTSNPD | | |
| TGFβRII | 289 | MGRGLLRGLWPLHI | | |
| | | VLWTRIASTIPPHV | | |
| | | QKSDVEMEAQKDEI | | |
| | | ICPSCNRTAHPLRH | | |
| | | INNDMIVTDNNGAV | | |
| | | KFPQLCKFCDVRKS | | |
| | | TCDNQKSCMSNCSI | | |
| | | TSICEKPQEVCVAV | | |
| | | WRKNDENITLETVC | | |
| | | HDPKLPYHDFILED | | |
| | | AASPKCIMKEKKKP | | |
| | | GETFFMCSCSSDEC | | |
| | | NDNIIFSEEYNTSN | | |
| | | PDLLLVIFQVTGIS | | |
| | | LLPPLGVAISVIII | | |
| | | FYCYRVNRQQKLSS | | |
| | | TWETGKTRKLMEFS | | |
| | | EHCAIILEDDRSDI | | |
| | | SSTCANNINHNTEL | | |
| | | LPIELDTLVGKGRF | | |
| | | AEVYKAKLKQNTSE | | |
| | | QFETVAVKIFPYEE | | |
| | | YASWKTEKDIFSDI | | |
| | | NLKHENILQFLTAE | | |
| | | ERKTELGKQYWLIT | | |
| | | AFHAKGNLQEYLTR | | |
| | | HVISWEDLRKLGSS | | |
| | | LARGIAHLHSDHTP | | |
| | | CGRPKMPIVHRDLK | | |
| | | SSNILVKNDLTCCL | | |
| | | CDFGLSLRLDPTLS | | |
| | | VDDLANSGQVGTAR | | |
| | | YMAPEVLESRMNLE | | |
| | | NVESFKQTDVYSMA | | |
| | | LVLWEMTSRCNAVG | | |
| | | EVKDYEPPFGSKVR | | |
| | | EHPCVESMKDNVLR | | |
| | | DRGRPEIPSFWLNH | | |
| | | QGIQMVCETLTECW | | |
| | | DHDPEARLTAQCVA | | |
| | | ERFSELEHLDRLSG | | |
| | | RSCSEEKIPEDGSL | | |
| | | NTTK | | |
| TGFβRII | 290 | MGRGLLRGLWPLHI | | |
| Isoform | | VLWTRIASTIPPHV | | |
| B | | QKSVNNDMIVTDNN | | |
| | | GAVKFPQLCKFCDV | | |
| | | RFSTCDNQKSCMSN | | |
| | | CSITSICEKPQEVC | | |
| | | VAVWRKNDENITLE | | |
| | | TVCHDPKLPYHDFI | | |
| | | LEDAASPKCIMKEK | | |
| | | KKPGETFFMCSCSS | | |
| | | DECNDNIIFSEEYN | | |
| | | TSNPDLLLVIFQVT | | |
| | | GISLLPPLGVAISV | | |
| | | IIIFYCYRVNRQQK | | |
| | | LSSTWETGKTRKLM | | |
| | | EFSEHCAIILEDDR | | |
| | | SDISSTCANNINHN | | |
| | | TELLPIELDTLVGK | | |
| | | GRFAEVYKAKLKQN | | |
| | | TSEQFETVAVKIFP | | |
| | | YEEYASWKTEKDIF | | |
| | | SDINLKHENILQFL | | |
| | | TAEERKTELGKQYW | | |
| | | LITAFHAKGNLQEY | | |
| | | LTRHVISWEDLRKL | | |
| | | GSSLARGIAHLHSD | | |
| | | HTPCGRPKMPIVHR | | |
| | | DLKSSNILVKNDLT | | |
| | | CCLCDFGLSLRLDP | | |
| | | TLSVDDLANSGQVG | | |
| | | TARYMAPEVLESRM | | |
| | | NLENVESFKQTDVY | | |
| | | SMALVLWEMTSRCN | | |
| | | AVGEVKDYEPPFGS | | |
| | | KVREHPCVESMKDN | | |
| | | VLRDRGRPEIPSFW | | |
| | | LNHQGIQMVCETLT | | |
| | | ECWDHDPEARLTAQ | | |
| | | CVAERFSELEHLDR | | |
| | | LSGRSCSEEKIPED | | |
| | | GSLNTTK | | |
| Anti- | 15 | EIVLTQSPATLSLS | 299 | GAGATCGTCCTGACACAGAGTCCAGCA |
| PD- | | PGERATLSCRASQS | | ACTCTGAGCCTGTCCCCCGGCGAACGA |
| 1(VL5) | | VSSYLAWYQQKPGQ | | GCTACTCTGTCCTGCCGGGCATCTCAG |
| IgG4- | | APRLLIYDASNRAT | | AGTGTGTCTAGTTACCTGGCCTGGTAT |
| (light | | GIPARFSGSGSGTD | | CAGCAGAAGCCCGGCCAGGCTCCTAGG |
| chain) | | FTLTISSLEPEDFA | | CTGCTGATCTACGACGCCAGCAACAGA |
| | | VYYCQQSSNWPRTF | | GCTACCGGGATTCCTGCCAGGTTCTCA |
| | | GQGTKVEIKRTVAA | | GGCAGCGGGTCCGGAACAGACTTTACC |
| | | PSVFIFPPSDEQLK | | CTGACAATCTCAAGCCTGGAGCCCGAA |
| | | SGTASVVCLLNNFY | | GATTTCGCTGTGTACTATTGCCAGCAG |
| | | PREAKVQWKVDNAL | | TCCTCTAATTGGCCTCGCACCTTTGGC |
| | | QSGNSQESVTEQDS | | CAGGGGACAAAGGTCGAGATCAAGCGT |
| | | KDSTYSLSSTLTLS | | ACGGTGGCTGCACCATCTGTCTTCATC |
| | | KADYEKHKVYACEV | | TTCCCGCCATCTGATGAGCAGTTGAAA |
| | | THQGLSSPVTKSFN | | TCTGGAACTGCCTCTGTTGTGTGCCTG |
| | | RGEC | | CTGAATAACTTCTATCCCAGAGAGGCC |
| | | | | AAAGTACAGTGGAAGGTGGATAACGCC |
| | | | | CTCCAATCGGGTAACTCCCAGGAGAGT |
| | | | | GTCACAGAGCAGGACAGCAAGGACAGC |
| | | | | ACCTACAGCCTCAGCAGCACCCTGACG |
| | | | | CTGAGCAAAGCAGACTACGAGAAACAC |
| | | | | AAAGTCTACGCCTGCGAAGTCACCCAT |
| | | | | CAGGGCCTGAGCTCGCCCGTCACAAAG |
| | | | | AGCTTCAACAGGGGAGAGTGT |
| Anti- | 296 | EIVLTQSPATLSLS | 300 | GAGATCGTCCTGACTCAGTCCCCAGCA |
| PD- | | PGERATLSCRASKG | | ACCCTGAGTCTGTCACCAGGAGAAAGG |
| 1(VL6) | | VSTSGYSYLHWYQQ | | GCAACCCTGAGCTGCCGAGCATCCAAG |
| | | KPGQAPRLLIYLAS | | GGGGTGAGCACATCCGGATACTCTTAT |
| IgG4- | | YLESGVPARFSGSG | | CTGCACTGGTACCAGCAGAAACCCGGA |
| (light | | SGTDFTLTISSLEP | | CAGGCTCCTCGACTGCTGATCTACCTG |
| chain) | | EDFAVYYCQHSRDL | | GCATCTTATCTGGAGAGTGGCGTGCCT |
| | | PLTFGGGTKVEIKR | | GCTCGGTTCTCTGGGAGTGGATCAGGC |
| | | TVAAPSVFIFPPSD | | ACCGATTTTACACTGACTATTTCTAGT |
| | | EQLKSGTASVVCLL | | CTGGAGCCAGAAGATTTCGCAGTGTAC |
| | | NNFYPREAKVQWKV | | TATTGCCAGCATTCTCGAGACCTGCCC |
| | | DNALQSGNSQESVT | | CTGACATTTGGCGGGGGAACTAAGGTC |
| | | EQLKSGTASVVCLL | | GAGATCAAACGTACGGTGGCTGCACCA |
| | | LTLSKADYEKHKVY | | TCTGTCTTCATCTTCCCGCCATCTGAT |
| | | ACEVTHQGLSSPVT | | GAGCAGTTGAAATCTGGAACTGCCTCT |
| | | KSFNRGEC | | GTTGTGTGCCTGCTGAATAACTTCTAT |
| | | | | CCCAGAGAGGCCAAAGTACAGTGGAAG |
| | | | | GTGGATAACGCCCTCCAATCGGGTAAC |
| | | | | TCCCAGGAGAGTGTCACAGAGCAGGAC |
| | | | | AGCAAGGACAGCACCTACAGCCTCAGC |
| | | | | AGCACCCTGACGCTGAGCAAAGCAGAC |
| | | | | TACGAGAAACACAAAGTCTACGCCTGC |
| | | | | GAAGTCACCCATCAGGGCCTGAGCTCG |
| | | | | CCCGTCACAAAGAGCTTCAACAGGGGA |
| | | | | GAGTGT |
| Anti- | 16 | QVQLVESGGGVVQP | 113 | CAGGTGCAGCTGGTCGAAAGCGGAGGA |
| PD-1 | | GRSLRLDCKASGIT | | GGAGTGGTCCAGCCAGGACGATCCCTG |
| (VH6) | | FSNSGMHWVRQAPG | | AGACTGGATTGTAAGGCCTCTGGAATC |
| IgG4- | | KGLEWVAVIWYDGS | | ACATTCTCTAACAGTGGAATGCACTGG |
| linker- | | KRYYADSVKGRFTI | | GTGCGCCAGGCACCAGGAAAAGGACTG |
| TGFβRII | | SRDNSKNTLFLQMN | | GAGTGGGTGGCCGTCATCTGGTACGAC |
| | | SLRAEDTAVYYCAT | | GGGTCAAAGCGATACTATGCAGATAGC |
| | | NDDYWGQGTLVTVS | | GTGAAAGGAAGGTTCACAATTTCACGC |
| | | SASTKGPSVFPLAP | | GACAACAGCAAGAATACTCTGTTTCTG |
| | | CSRSTSESTAALGC | | CAGATGAACTCTCTGAGAGCAGAGGAT |
| | | LVKDYFPEPVTVSW | | ACTGCCGTGTACTATTGTGCTACCAAT |
| | | NSGALTSGVHTFPA | | GACGATTATTGGGGGCAGGGAACTCTG |
| | | VLQSSGLYSLSSVV | | GTGACCGTCAGTTCAGCTAGCACCAAG |
| | | TVPSSSLGTKTYTC | | GGCCCATCGGTCTTCCCCCTGGCGCCC |
| | | NVDHKPSNTKVDKR | | TGCTCCAGGAGCACCTCCGAGAGCACA |
| | | VESKYGPPCPSCPA | | GCCGCCCTGGGCTGCCTGGTCAAGGAC |
| | | PEFLGGPSVFLFPP | | TACTTCCCCGAACCGGTGACGGTGTCG |
| | | KPKDTLMISRTPEV | | TGGAACTCAGGCGCCCTGACCAGCGGC |
| | | TCVVVDVSQEDPEV | | GTGCACACCTTCCCGGCTGTCCTACAG |
| | | QFNWYVDGVEVHNA | | TCCTCAGGACTCTACTCCCTCAGCAGC |
| | | KTKPREEQFNSTYR | | GTGGTGACCGTGCCCTCCAGCAGCTTG |
| | | VVSVLTVLHQDWLN | | GGCACGAAGACCTACACCTGCAACGTA |
| | | GKEYKCKVSNKGLP | | GATCACAAGCCCAGCAACACCAAGGTG |
| | | SSIEKTISKAKGQP | | GACAAGAGAGTTGAGTCCAAATATGGT |
| | | REPQVYTLPPSQEE | | CCCCCATGCCCATCATGCCCAGCACCT |
| | | MTKNQVSLTCLVKG | | GAGTTCCTGGGGGGACCATCAGTCTTC |
| | | FYPSDIAVEWESNG | | CTGTTCCCCCCAAAACCCAAGGACACT |
| | | QPENNYKTTPPVLD | | CTCATGATCTCCCGGACCCCTGAGGTC |
| | | SDGSFFLYSRLTVD | | ACGTGCGTGGTGGTGGACGTGAGCCAG |
| | | KSRWQEGVNFSCSV | | GAAGACCCCGAGGTCCAGTTCAACTGG |
| | | MHEALHNHYTQKSL | | TACGTGGATGGCGTGGAGGTGCATAAT |
| | | SLSPGKGGGGSGGG | | GCCAAGACAAAGCCGCGGGAGGAGCAG |
| | | GSIPPHVQKSVNND | | TTCAACAGCACGTACCGTGTGGTCAGC |
| | | MIVTDNNGAVKFPQ | | GTCCTCACCGTCCTGCACCAGGACTGG |
| | | LCKFCDVRFSTCDN | | CTGAACGGCAAGGAGTACAAGTGCAAG |
| | | QKSCMSNCSITSIC | | GTCTCCAACAAAGGCCTCCCGTCCTCC |
| | | EKPQEVCVAVWRKN | | ATCGAGAAAACCATCTCCAAAGCCAAA |
| | | DENITLETVCHDPK | | GGGCAGCCCCGAGAGCCACAGGTGTAC |
| | | LPYHDFILEDAASP | | ACCCTGCCCCCATCCCAGGAGGAGATG |
| | | KCIMKEKKKPGETF | | ACCAAGAACCAGGTCAGCCTGACCTGC |
| | | FMCSCSSDECNDNI | | CTGGTCAAAGGCTTCTACCCCAGCGAC |
| | | IFSEEYNTSNPD | | ATCGCCGTGGAGTGGGAGAGCAATGGG |
| | | | | CAGCCGGAGAACAACTACAAGACCACG |
| | | | | CCTCCCGTGCTGGACTCCGACGGCTCC |
| | | | | TTCTTCCTCTACAGCAGGCTCACCGTG |
| | | | | GACAAGAGCAGGTGGCAGGAGGGGAAT |
| | | | | GTCTTCTCATGCTCCGTGATGCATGAG |
| | | | | GCTCTGCACAACCACTACACACAGAAG |
| | | | | AGCCTCTCCCTGTCTCCGGGTAAAGGT |
| | | | | GGAGGTGGTTCTGGAGGTGGAGGTAGT |
| | | | | ATCCCTCCTCACGTACAGAAGTCCGTG |
| | | | | AACAATGACATGATTGTCACTGACAAT |
| | | | | AACGGAGCCGTCAAGTTTCCTCAGCTA |
| | | | | TGTAAGTTCTGCGATGTTCGGTTCTCC |
| | | | | ACATGCGATAATCAGAAAAGCTGTATG |
| | | | | TCTAATTGCAGTATCACTAGTATATGC |
| | | | | GAAAAACCTCAAGAAGTTTGCGTCGCC |
| | | | | GTGTGGCGGAAAAATGATGAAAATATC |
| | | | | ACGCTTGAGACTGTCTGCCATGATCCA |
| | | | | AAGTTACCCTACCACGACTTCATCTTA |
| | | | | GAAGACGCCGCATCACCCAAGTGCATT |
| | | | | ATGAAAGAGAAAAAGAAGCCAGGAGAA |
| | | | | ACATTCTTTATGTGCTCATGCTCCTCT |
| | | | | GACGAATGCAACGACAACATTATCTTC |
| | | | | TCTGAGGAGTATAACACCTCAAATCCA |
| | | | | GAC |
| Anti- | 143 | QVQLVESGGGVVQP | 114 | CAGGTGCAGCTGGTCGAAAGCGGAGGA |
| PD-1 | | GRSLRLDCKASGIT | | GGAGTGGTCCAGCCAGGACGATCCCTG |
| (VH6) | | FSNSGMHWVRQAPG | | AGACTGGATTGTAAGGCCTCTGGAATC |
| IgG4- | | KGLEWVAVIWYDGS | | ACATTCTCTAACAGTGGAATGCACTGG |
| (S108P). | | KRYYADSVKGRFTI | | GTGCGCCAGGCACCAGGAAAAGGACTG |
| linker- | | SRDNSKNTLFLQMN | | GAGTGGGTGGCCGTCATCTGGTACGAC |
| TGFβRII | | SLRAEDTAVYYCAT | | GGGTCAAAGCGATACTATGCAGATAGC |
| | | NDDYWGQGTLVTVS | | GTGAAAGGAAGGTTCACAATTTCACGC |
| | | SASTKGPSVFPLAP | | GACAACAGCAAGAATACTCTGTTTCTG |
| | | CSRSTSESTAALGC | | CAGATGAACTCTCTGAGAGCAGAGGAT |
| | | LVKDYFPEPVTVSW | | ACTGCCGTGTACTATTGTGCTACCAAT |
| | | NSGALTSGVHTFPA | | GACGATTATTGGGGGCAGGGAACTCTG |
| | | VLQSSGLYSLSSVV | | GTGACCGTCAGTTCAGCTAGCACCAAG |
| | | TVPSSSLGTKTYTC | | GGCCCATCGGTCTTCCCCCTGGCGCCC |
| | | NVDHKPSNTKVDKR | | TGCTCCAGGAGCACCTCCGAGAGCACA |
| | | VESKYGPPCPPCPA | | GCCGCCCTGGGCTGCCTGGTCAAGGAC |
| | | PEFLGGPSVFLFPP | | TACTTCCCCGAACCGGTGACGGTGTCG |
| | | KPKDTLMISRTPEV | | TGGAACTCAGGCGCCCTGACCAGCGGC |
| | | TCVVVDVSQESPEV | | GTGCACACCTTCCCGGCTGTCCTACAG |
| | | QFNWYVDGVEVHNA | | TCCTCAGGACTCTACTCCCTCAGCAGC |
| | | KTKPREEQFNSTYR | | GTGGTGACCGTGCCCTCCAGCAGCTTG |
| | | VVSVLTVLHQDWLN | | GGCACGAAGACCTACACCTGCAACGTA |
| | | GKEYKCKVSNKGLP | | GATCACAAGCCCAGCAACACCAAGGTG |
| | | SSIEKTISKAKGQP | | GACAAGAGAGTTGAGTCCAAATATGGT |
| | | REPQVYTLPPSQEE | | CCCCCATGCCCACCATGCCCAGCACCT |
| | | MTKNQVSLTCLVKG | | GAGTTCCTGGGGGGACCATCAGTCTTC |
| | | FYPSDIAVEWESNG | | CTGTTCCCCCCAAAACCCAAGGACACT |
| | | QPENNYKTTPPVLD | | CTCATGATCTCCCGGACCCCTGAGGTC |
| | | SDGSFFLYSRLTVD | | ACGTGCGTGGTGGTGGACGTGAGCCAG |
| | | KSRWQEGNVFSCSV | | GAAGACCCCGAGGTCCAGTTCAACTGG |
| | | MHEALHNHYTQKSL | | TACGTGGATGGCGTGGAGGTGCATAAT |
| | | SLSPGKGGGGSGGG | | GCCAAGACAAAGCCGCGGGAGGAGCAG |
| | | GSIPPHVQKSVNND | | TTCAACAGCACGTACCGTGTGGTCAGC |
| | | MIVTDNNGAVKFPQ | | GTCCTCACCGTCCTGCACCAGGACTGG |
| | | LCKFCDVRFSTCDN | | CTGAACGGCAAGGAGTACAAGTGCAAG |
| | | QKSCMSNCSITSIC | | GTCTCCAACAAAGGCCTCCCGTCCTCC |
| | | EKPQEVCVAVWRKN | | ATCGAGAAAACCATCTCCAAAGCCAAA |
| | | DENITLETVCHDPK | | GGGCAGCCCCGAGAGCCACAGGTGTAC |
| | | LPYHDFILEDAASP | | ACCCTGCCCCCATCCCAGGAGGAGATG |
| | | KCIMKEKKKPGETF | | ACCAAGAACCAGGTCAGCCTGACCTGC |
| | | FMCSCSSDECNDNI | | CTGGTCAAAGGCTTCTACCCCAGCGAC |
| | | IFSEEYNTSNPD | | ATCGCCGTGGAGTGGGAGAGCAATGGG |
| | | | | CAGCCGGAGAACAACTACAAGACCACG |
| | | | | CCTCCCGTGCTGGACTCCGACGGCTCC |
| | | | | TTCTTCCTCTACAGCAGGCTCACCGTG |
| | | | | GACAAGAGCAGGTGGCAGGAGGGGAAT |
| | | | | GTCTTCTCATGCTCCGTGATGCATGAG |
| | | | | GCTCTGCACAACCACTACACACAGAAG |
| | | | | AGCCTCTCCCTGTCTCCGGGTAAAGGT |
| | | | | GGAGGTGGTTCTGGAGGTGGAGGTAGT |
| | | | | ATCCCTCCTCACGTACAGAAGTCCGTG |
| | | | | AACAATGACATGATTGTCACTGACAAT |
| | | | | AACGGAGCCGTCAAGTTTCCTCAGCTA |
| | | | | TGTAAGTTCTGCGATGTTCGGTTCTCC |
| | | | | ACATGCGATAATCAGAAAAGCTGTATG |
| | | | | TCTAATTGCAGTATCACTAGTATATGC |
| | | | | GAAAAACCTCAAGAAGTTTGCGTCGCC |
| | | | | GTGTGGCGGAAAAATGATGAAAATATC |
| | | | | ACGCTTGAGACTGTCTGCCATGATCCA |
| | | | | AAGTTACCCTACCACGACTTCATCTTA |
| | | | | GAAGACGCCGCATCACCCAAGTGCATT |
| | | | | ATGAAAGAGAAAAAGAAGCCAGGAGAA |
| | | | | ACATTCTTTATGTGCTCATGCTCCTCT |
| | | | | GACGAATGCAACGACAACATTATCTTC |
| | | | | TCTGAGGAGTATAACACCTCAAATCCA |
| | | | | GAC |
| Anti- | 144 | QVQLVQSGVEVKKP | 115 | CAGGTGCAGCTGGTCCAGAGCGGCGTC |
| PD-1 | | GASVKVSCKASGYT | | GAAGTCAAGAAACCCGGGGCCTCAGTG |
| VH7 | | FTNYYMYWVRQAPG | | AAGGTCAGCTGTAAAGCTTCCGGCTAC |
| IgG4 | | QGLEWMGGINPSNG | | ACCTTCACAAACTACTATATGTATTGG |
| (S108P) | | GTNFNEKFKNRVTL | | GTGAGACAGGCACCAGGACAGGGACTG |
| linker- | | TTDSSTTTAYMELK | | GAGTGGATGGGCGGGATTAACCCTAGT |
| TGFbRII | | SLQFDDTAVYYCAR | | AATGGAGGCACTAACTTCAACGAAAAG |
| | | RDYRFDMGFDYWGQ | | TTTAAAAACAGGGTGACCCTGACCACA |
| | | GTTVTVSSASTKGP | | GATTCAAGCACTACCACAGCTTACATG |
| | | SVFPLAPCSRSTSE | | GAGCTGAAGTCCCTGCAGTTTGACGAT |
| | | STAALGCLVKDYFP | | ACAGCCGTGTACTATTGTGCTCGGAGA |
| | | EPVTVSWNSGALTS | | GACTACAGGTTCGATATGGGCTTTGAC |
| | | GVHTFPAVLQSSGL | | TATTGGGGCCAGGGGACTACCGTGACC |
| | | YSLSSVVTVPSSSL | | GTCTCCTCTGCTAGCACCAAGGGCCCA |
| | | GTKTYTCNVDHKPS | | TCGGTCTTCCCCCTGGCGCCCTGCTCC |
| | | NTKVDKRVESKYGP | | AGGAGCACCTCCGAGAGCACAGCCGCC |
| | | PCPPCPAPEFLGGP | | CTGGGCTGCCTGGTCAAGGACTACTTC |
| | | SVFLFPPKPKDTLM | | CCCGAACCGGTGACGGTGTCGTGGAAC |
| | | ISRTPEVTCVVVDV | | TCAGGCGCCCTGACCAGCGGCGTGCAC |
| | | SQESPEVQFNWYVD | | ACCTTCCCGGCTGTCCTACAGTCCTCA |
| | | GVEVHNAKTKPREE | | GGACTCTACTCCCTCAGCAGCGTGGTG |
| | | QFNSTYRVVSVLTV | | ACCGTGCCCTCCAGCAGCTTGGGCACG |
| | | LHQDWLNGKEYKCK | | AAGACCTACACCTGCAACGTAGATCAC |
| | | VSNKGLPSSIEKTI | | AAGCCCAGCAACACCAAGGTGGACAAG |
| | | SKAKGQPREPQVYT | | AGAGTTGAGTCCAAATATGGTCCCCCA |
| | | LPPSQEEMTKNQVS | | TGCCCACCATGCCCAGCACCTGAGTTC |
| | | LTCLVKGFYSPDIA | | CTGGGGGGACCATCAGTCTTCCTGTTC |
| | | VEWESNGQPENNYK | | CCCCCAAAACCCAAGGACACTCTCATG |
| | | TTPPVLDSDGSFFL | | ATCTCCCGGACCCCTGAGGTCACGTGC |
| | | YSRLTVDKSRWQEG | | GTGGTGGTGGACGTGAGCCAGGAAGAC |
| | | NVFSCSVMHEALHN | | CCCGAGGTCCAGTTCAACTGGTACGTG |
| | | HYTQKSLSLSPGKG | | GATGGCGTGGAGGTGCATAATGCCAAG |
| | | GGGSGGGGSIPPHV | | ACAAAGCCGCGGGAGGAGCAGTTCAAC |
| | | QKSVNNDMIVTDNN | | AGCACGTACCGTGTGGTCAGCGTCCTC |
| | | GAVKFPQLCKFCDV | | ACCGTCCTGCACCAGGACTGGCTGAAC |
| | | RFSTCDNQKSCMSN | | GGCAAGGAGTACAAGTGCAAGGTCTCC |
| | | CSITSICEKPQEVC | | AACAAAGGCCTCCCGTCCTCCATCGAG |
| | | VAVWRKNDENITLE | | AAAACCATCTCCAAAGCCAAAGGGCAG |
| | | TVCHDPKLPYHDFI | | CCCCGAGAGCCACAGGTGTACACCCTG |
| | | LEDAASPKCIMKEK | | CCCCCATCCCAGGAGGAGATGACCAAG |
| | | KKPGETFFMCSCSS | | AACCAGGTCAGCCTGACCTGCCTGGTC |
| | | DECNDNIIFSEEYN | | AAAGGCTTCTACCCCAGCGACATCGCC |
| | | TSNPD* | | GTGGAGTGGGAGAGCAATGGGCAGCCG |
| | | | | GAGAACAACTACAAGACCACGCCTCCC |
| | | | | GTGCTGGACTCCGACGGCTCCTTCTTC |
| | | | | CTCTACAGCAGGCTCACCGTGGACAAG |
| | | | | AGCAGGTGGCAGGAGGGGAATGTCTTC |
| | | | | TCATGCTCCGTGATGCATGAGGCTCTG |
| | | | | CACAACCACTACACACAGAAGAGCCTC |
| | | | | TCCCTGTCTCCGGGTAAAGGTGGAGGT |
| | | | | GGTTCTGGAGGTGGAGGTAGTATCCCT |
| | | | | CCTCACGTACAGAAGTCCGTGAACAAT |
| | | | | GACATGATTGTCACTGACAATAACGGA |
| | | | | GCCGTCAAGTTTCCTCAGCTATGTAAG |
| | | | | TTCTGCGATGTTCGGTTCTCCACATGC |
| | | | | GATAATCAGAAAAGCTGTATGTCTAAT |
| | | | | TGCAGTATCACTAGTATATGCGAAAAA |
| | | | | CCTCAAGAAGTTTGCGTCGCCGTGTGG |
| | | | | CGGAAAAATGATGAAAATATCACGCTT |
| | | | | GAGACTGTCTGCCATGATCCAAAGTTA |
| | | | | CCCTACCACGACTTCATCTTAGAAGAC |
| | | | | GCCGCATCACCCAAGTGCATTATGAAA |
| | | | | GAGAAAAAGAAGCCAGGAGAAACATTC |
| | | | | TTTATGTGCTCATGCTCCTCTGACGAA |
| | | | | TGCAACGACAACATTATCTTCTCTGAG |
| | | | | GAGTATAACACCTCAAATCCAGAC |
| Anti- | 145 | QVQLVQSGVEVKKP | 116 | CAGGTGCAGCTGGTCCAGAGCGGCGTG |
| PD-1 | | GASVKVSCKASGYT | | GAAGTCAAGAAACCCGGGGCCTCAGTG |
| VH7 | | FTNYYMYWVRQAPC | | AAGGTCAGCTGTAAAGCTTCCGGCTAC |
| IgG4 | | QGLEWMGGINPSNG | | ACCTTCACAAACTACTATATGTATTGG |
| (linker- | | GTNFNEKFKNRVTL | | GTGAGACAGGCACCAGGACAGGGACTG |
| TGFbRII | | TTDSSTTTAYMELK | | GAGTGGATGGGCGGGATTAACCCTAGT |
| | | SLQFDDTAVYYCAR | | AATGGAGGCACTAACTTCAACGAAAAG |
| | | RDYRFDMGFDYWGQ | | TTTAAAAACAGGGTGACCCTGACCACA |
| | | GTTVTVSSASTKGP | | GATTCAAGCACTACCACAGCTTACATG |
| | | SVFPLAPCSRSTSE | | GAGCTGAAGTCCCTGCAGTTTGACGAT |
| | | STAALGCLVKDYFP | | ACAGCCGTGTACTATTGTGCTCGGAGA |
| | | EPVTVSWNSGALTS | | GACTACAGGTTCGATATGGGCTTTGAC |
| | | GVHTFPAVLQSSGL | | TATTGGGGCCAGGGGACTACCGTGACC |
| | | YSLSSVVTVPSSSL | | GTCTCCTCTGCTAGCACCAAGGGCCCA |
| | | GTKTYTCNVDHKPS | | TCGGTCTTCCCCCTGGCGCCCTGCTCC |
| | | NTKVDKRVESKYGP | | AGGAGCACCTCCGAGAGCACAGCCGCC |
| | | PCPSCPAPEFLGGP | | CTGGGCTGCCTGGTCAAGGACTACTTC |
| | | SVFLFPPKPKDTLM | | CCCGAACCGGTGACGGTGTCGTGGAAC |
| | | ISRTPEVTCVVVDV | | TCAGGCGCCCTGACCAGCGGCGTGCAC |
| | | SQEDPEVQFNWYVD | | ACCTTCCCGGCTGTCCTACAGTCCTCA |
| | | GVEVHNAKTKPREE | | GGACTCTACTCCCTCAGCAGCGTGGTG |
| | | QFNSTYRVVSVLTV | | ACCGTGCCCTCCAGCAGCTTGGGCACG |
| | | LHQDWLNGKEYKCK | | AAGACCTACACCTGCAACGTAGATCAC |
| | | VSNKGLPSSIEKTI | | AAGCCCAGCAACACCAAGGTGGACAAG |
| | | SKAKGQPREPQVYT | | AGAGTTGAGTCCAAATATGGTCCCCCA |
| | | LPPSQEEMTKNQVS | | TGCCCATCATGCCCAGCACCTGAGTTC |
| | | LTCLVKGFYPSDIA | | CTGGGGGGACCATCAGTCTTCCTGTTC |
| | | VEWESNGQPENNYK | | CCCCCAAAACCCAAGGACACTCTCATG |
| | | TTPPVLDSDGSFFL | | ATCTCCCGGACCCCTGAGGTCACGTGC |
| | | YSRLTVDKSRWQEG | | GTGGTGGTGGACGTGAGCCAGGAAGAC |
| | | NVFSCSVMHEALHN | | CCCGAGGTCCAGTTCAACTGGTACGTG |
| | | HYTQKSLSLSPGKG | | GATGGCGTGGAGGTGCATAATGCCAAG |
| | | GGGSGGGGSIPPHV | | ACAAAGCCGCGGGAGGAGCAGTTCAAC |
| | | QKSVNNDMIVTDNN | | AGCACGTACCGTGTGGTCAGCGTCCTC |
| | | GAVKFPQLCKFCDV | | ACCGTCCTGCACCAGGACTGGCTGAAC |
| | | RFSTCDNQKSCMSN | | GGCAAGGAGTACAAGTGCAAGGTCTCC |
| | | CSITSICEKPQEVC | | AACAAAGGCCTCCCGTCCTCCATCGAG |
| | | VAVWRKNDENITLE | | AAAACCATCTCCAAAGCCAAAGGGCAG |
| | | TVCHDPKLPYHDFI | | CCCCGAGAGCCACAGGTGTACACCCTG |
| | | LEDAASPKCIMKEK | | CCCCCATCCCAGGAGGAGATGACCAAG |
| | | KKPGETFFMCSCSS | | AACCAGGTCAGCCTGACCTGCCTGGTC |
| | | DECNDNIIFSEEYN | | AAAGGCTTCTACCCCAGCGACATCGCC |
| | | TSNPD | | GTGGAGTGGGAGAGCAATGGGCAGCCG |
| | | | | GAGAACAACTACAAGACCACGCCTCCC |
| | | | | GTGCTGGACTCCGACGGCTCCTTCTTC |
| | | | | CTCTACAGCAGGCTCACCGTGGACAAG |
| | | | | AGCAGGTGGCAGGAGGGGAATGTCTTC |
| | | | | TCATGCTCCGTGATGCATGAGGCTCTG |
| | | | | CACAACCACTACACACAGAAGAGCCTC |
| | | | | TCCCTGTCTCCGGGTAAAGGTGGAGGT |
| | | | | GGTTCTGGAGGTGGAGGTAGTATCCCT |
| | | | | CCTCACGTACAGAAGTCCGTGAACAAT |
| | | | | GACATGATTGTCACTGACAATAACGGA |
| | | | | GCCGTCAAGTTTCCTCAGCTATGTAAG |
| | | | | TTCTGCGATGTTCGGTTCTCCACATGC |
| | | | | GATAATCAGAAAAGCTGTATGTCTAAT |
| | | | | TGCAGTATCACTAGTATATGCGAAAAA |
| | | | | CCTCAAGAAGTTTGCGTCGCCGTGTGG |
| | | | | CGGAAAAATGATGAAAATATCACGCTT |
| | | | | GAGACTGTCTGCCATGATCCAAAGTTA |
| | | | | CCCTACCACGACTTCATCTTAGAAGAC |
| | | | | GCCGCATCACCCAAGTGCATTATGAAA |
| | | | | GAGAAAAAGAAGCCAGGAGAAACATTC |
| | | | | TTTATGTGCTCATGCTCCTCTGACGAA |
| | | | | TGCAACGACAACATTATCTTCTCTGAG |
| | | | | GAGTATAACACCTCAAATCCAGAC |
| VH6- | 294 | QVQLVESGGGVVQP | 297 | CAGGTGCAGCTGGTCGAAAGCGGAGGA |
| IgG4 | | GRSLRLDCKASGIT | | GGAGTGGTCCAGCCAGGACGATCCCTG |
| (mut)- | | FSNSGMHWVRQAPG | | AGACTGGATTGTAAGGCCTCTGGAATC |
| linker- | | KGLEWVAVIWYDGS | | ACATTCTCTAACAGTGGAATGCACTGG |
| ECD | | KRYYADSVKGRFTI | | GTGCGCCAGGCACCAGGAAAAGGACTG |
| | | SRDNSKNTLFLQMN | | GAGTGGGTGGCCGTCATCTGGTACGAC |
| | | SLRAEDTAVYYCAT | | GGGTCAAAGCGATACTATGCAGATAGC |
| | | NDDYWGQGTLVTVS | | GTGAAAGGAAGGTTCACAATTTCACGC |
| | | SASTKGPSVFPLAP | | GACAACAGCAAGAATACTCTGTTTCTG |
| | | CSRSTSESTAALGC | | CAGATGAACTCTCTGAGAGCAGAGGAT |
| | | LVKDYFPEPVTVSW | | ACTGCCGTGTACTATTGTGCTACCAAT |
| | | NSGALTSGVHTFPA | | GACGATTATTGGGGGCAGGGAACTCTG |
| | | VLQSSGLYSLSSVV | | GTGACCGTCAGTTCAGCTAGCACCAAG |
| | | TVPSSSLGTKTYTC | | GGCCCATCGGTCTTCCCCCTGGCGCCC |
| | | NVDHKPSNTKVDKR | | TGCTCCAGGAGCACCTCCGAGAGCACA |
| | | VESKYGPPCPPCPA | | GCCGCCCTGGGCTGCCTGGTCAAGGAC |
| | | PEFLGGPSVFLFPP | | TACTTCCCCGAACCGGTGACGGTGTCG |
| | | KPKDTLMISRTPEV | | TGGAACTCAGGCGCCCTGACCAGCGGC |
| | | TCVVVDVSQESPEV | | GTGCACACCTTCCCGGCTGTCCTACAG |
| | | QFNWYVDGVEVHNA | | TCCTCAGGACTCTACTCCCTCAGCAGC |
| | | KTKPREEQFNSTYR | | GTGGTGACCGTGCCCTCCAGCAGCTTG |
| | | VVSVLTVLHQDWLN | | GGCACGAAGACCTACACCTGCAACGTA |
| | | GKEYKCKVSNKGLP | | GATCACAAGCCCAGCAACACCAAGGTG |
| | | SSIEKTISKAKGQP | | GACAAGAGAGTTGAGTCCAAATATGGT |
| | | REPQVYTLPPSQEE | | CCCCCATGCCCACCATGCCCAGCACCT |
| | | MTKNQVSLTCLVKG | | GAGTTCCTGGGGGGACCATCAGTCTTC |
| | | FYPSDIAVEWESNG | | CTGTTCCCCCCAAAACCCAAGGACACT |
| | | QPENNYKTTPPVLD | | CTCATGATCTCCCGGACCCCTGAGGTC |
| | | SDGSFFLYSRLTVD | | ACGTGCGTGGTGGTGGACGTGAGCCAG |
| | | KSRWQEGNVFSCSV | | GAAGACCCCGAGGTCCAGTTCAACTGG |
| | | MHEALHNHYTQKSL | | TACGTGGATGGCGTGGAGGTGCATAAT |
| | | SLSLGKGGGGSGGG | | GCCAAGACAAAGCCGCGGGAGGAGCAG |
| | | GSIPPHVQKSVNND | | TTCAACAGCACGTACCGTGTGGTCAGC |
| | | MIVTDNNGAVKFPQ | | GTCCTCACCGTCCTGCACCAGGACTGG |
| | | LCKFCDVRFSTCDN | | CTGAACGGCAAGGAGTACAAGTGCAAG |
| | | QKSCMSNCSITSIC | | GTCTCCAACAAAGGCCTCCCGTCCTCC |
| | | EKPQEVCVAVWRKN | | ATCGAGAAAACCATCTCCAAAGCCAAA |
| | | DENITLETVCHDPK | | GGGCAGCCCCGAGAGCCACAGGTGTAC |
| | | LPYHDFILEDAASP | | ACCCTGCCCCCATCCCAGGAGGAGATG |
| | | KCIMKEKKKPGETF | | ACCAAGAACCAGGTCAGCCTGACCTGC |
| | | FMCSCSSDECNDNI | | CTGGTCAAAGGCTTCTACCCCAGCGAC |
| | | IFSEEYNTSNPD | | ATCGCCGTGGAGTGGGAGAGCAATGGG |
| | | | | CAGCCGGAGAACAACTACAAGACCACG |
| | | | | CCTCCCGTGCTGGACTCCGACGGCTCC |
| | | | | TTCTTCCTCTACAGCAGGCTCACCGTG |
| | | | | GACAAGAGCAGGTGGCAGGAGGGGAAT |
| | | | | GTCTTCTCATGCTCCGTGATGCATGAG |
| | | | | GCTCTGCACAACCACTACACACAGAAG |
| | | | | AGCCTCTCCCTGTCTCTGGGTAAAGGT |
| | | | | GGAGGTGGTTCTGGAGGTGGAGGTAGT |
| | | | | ATCCCTCCTCACGTACAGAAGTCCGTG |
| | | | | AACAATGACATGATTGTCACTGACAAT |
| | | | | AACGGAGCCGTCAAGTTTCCTCAGCTA |
| | | | | TGTAAGTTCTGCGATGTTCGGTTCTCC |
| | | | | ACATGCGATAATCAGAAAAGCTGTATG |
| | | | | TCTAATTGCAGTATCACTAGTATATGC |
| | | | | GAAAAACCTCAAGAAGTTTGCGTCGCC |
| | | | | GTGTGGCGGAAAAATGATGAAAATATC |
| | | | | ACGCTTGAGACTGTCTGCCATGATCCA |
| | | | | AAGTTACCCTACCACGACTTCATCTTA |
| | | | | GAAGACGCCGCATCACCCAAGTGCATT |
| | | | | ATGAAAGAGAAAAAGAAGCCAGGAGAA |
| | | | | ACATTCTTTATGTGCTCATGCTCCTCT |
| | | | | GACGAATGCAACGACAACATTATCTTC |
| | | | | TCTGAGGAGTATAACACCTCAAATCCA |
| | | | | GAC |
| VH7- | 295 | QVQLVQSGVEVKKP | 298 | CAGGTGCAGCTGGTCCAGAGCGGCGTG |
| IgG4 | | GASVKVSCKASGYT | | GAAGTCAAGAAACCCGGGGCCTCAGTG |
| (mut)- | | FTNYYMYWVRQAPG | | AAGGTCAGCTGTAAAGCTTCCGGCTAC |
| linker- | | QGLEWMGGINPSNG | | ACCTTCACAAACTACTATATGTATTGG |
| ECD | | GTNFNEKFKNRVTL | | GTGAGACAGGCACCAGGACAGGGACTG |
| | | TTDSSTTTAYMELK | | GAGTGGATGGGCGGGATTAACCCTAGT |
| | | SLQFDDTAVYYCAR | | AATGGAGGCACTAACTTCAACGAAAAG |
| | | RDYRFDMGFDYWGQ | | TTTAAAAACAGGGTGACCCTGACCACA |
| | | GTTVTVSSASTKGP | | GATTCAAGCACTACCACAGCTTACATG |
| | | SVFPLAPCSRSTSE | | GAGCTGAAGTCCCTGCAGTTTGACGAT |
| | | STAALGCLVKDYFP | | ACAGCCGTGTACTATTGTGCTCGGAGA |
| | | EPVTVSWNSGALTS | | GACTACAGGTTCGATATGGGCTTTGAC |
| | | GVHTFPAVLQSSGL | | TATTGGGGCCAGGGGACTACCGTGACC |
| | | YSLSSVVTVPSSSL | | GTCTCCTCTGCTAGCACCAAGGGCCCA |
| | | GTKTYTCNVDHKPS | | TCGGTCTTCCCCCTGGCGCCCTGCTCC |
| | | NTKVDKRVESKYGP | | AGGAGCACCTCCGAGAGCACAGCCGCC |
| | | PCPPCPAPEFLGGP | | CTGGGCTGCCTGGTCAAGGACTACTTC |
| | | SVFLFPPKPKDTLM | | CCCGAACCGGTGACGGTGTCGTGGAAC |
| | | ISRTPEVTCVVVDV | | TCAGGCGCCCTGACCAGCGGCGTGCAC |
| | | SQEDPEVQFNWYVD | | ACCTTCCCGGCTGTCCTACAGTCCTCA |
| | | GVEVHNAKTKPREE | | GGACTCTACTCCCTCAGCAGCGTGGTG |
| | | QFNSTYRVVSVLTV | | ACCGTGCCCTCCAGCAGCTTGGGCACG |
| | | LHQDWLNGKEYKCK | | AAGACCTACACCTGCAACGTAGATCAC |
| | | VSNKGLPSSIEKTI | | AAGCCCAGCAACACCAAGGTGGACAAG |
| | | SKAKGQPREPQVYT | | AGAGTTGAGTCCAAATATGGTCCCCCA |
| | | LPPSQEEMTKNQVS | | TGCCCACCATGCCCAGCACCTGAGTTC |
| | | LTCLVKGFYPSDIA | | CTGGGGGGACCATCAGTCTTCCTGTTC |
| | | VEWESNGQPENNYK | | CCCCCAAAACCCAAGGACACTCTCATG |
| | | TTPPVLDSDGSFFL | | ATCTCCCGGACCCCTGAGGTCACGTGC |
| | | YSRLTVDKSRWQEG | | GTGGTGGTGGACGTGAGCCAGGAAGAC |
| | | NVFSCSVMHEALHN | | CCCGAGGTCCAGTTCAACTGGTACGTG |
| | | HYTQKSLSLSLGKG | | GATGGCGTGGAGGTGCATAATGCCAAG |
| | | GGGSGGGGSIPPHV | | ACAAAGCCGCGGGAGGAGCAGTTCAAC |
| | | QKSVNNDMIVTDNN | | AGCACGTACCGTGTGGTCAGCGTCCTC |
| | | GAVKFPQLCKFCDV | | ACCGTCCTGCACCAGGACTGGCTGAAC |
| | | RFSTCDNQKSCMSN | | GGCAAGGAGTACAAGTGCAAGGTCTCC |
| | | CSITSICEKPQEVC | | AACAAAGGCCTCCCGTCCTCCATCGAG |
| | | VAVWRKNDENITLE | | AAAACCATCTCCAAAGCCAAAGGGCAG |
| | | TVCHDPKLPYHDFI | | CCCCGAGAGCCACAGGTGTACACCCTG |
| | | LEDAASPKCIMKEK | | CCCCCATCCCAGGAGGAGATGACCAAG |
| | | KKPGETFFMCSCSS | | AACCAGGTCAGCCTGACCTGCCTGGTC |
| | | DECNDNIIFSEEYN | | AAAGGCTTCTACCCCAGCGACATCGCC |
| | | TSNPD* | | GTGGAGTGGGAGAGCAATGGGCAGCCG |
| | | | | GAGAACAACTACAAGACCACGCCTCCC |
| | | | | GTGCTGGACTCCGACGGCTCCTTCTTC |
| | | | | CTCTACAGCAGGCTCACCGTGGACAAG |
| | | | | AGCAGGTGGCAGGAGGGGAATGTCTTC |
| | | | | TCATGCTCCGTGATGCATGAGGCTCTG |
| | | | | CACAACCACTACACACAGAAGAGCCTC |
| | | | | TCCCTGTCTCTGGGTAAAGGTGGAGGT |
| | | | | GGTTCTGGAGGTGGAGGTAGTATCCCT |
| | | | | CCTCACGTACAGAAGTCCGTGAACAAT |
| | | | | GACATGATTGTCACTGACAATAACGGA |
| | | | | GCCGTCAAGTTTCCTCAGCTATGTAAG |
| | | | | TTCTGCGATGTTCGGTTCTCCACATGC |
| | | | | GATAATCAGAAAAGCTGTATGTCTAAT |
| | | | | TGCAGTATCACTAGTATATGCGAAAAA |
| | | | | CCTCAAGAAGTTTGCGTCGCCGTGTGG |
| | | | | CGGAAAAATGATGAAAATATCACGCTT |
| | | | | GAGACTGTCTGCCATGATCCAAAGTTA |
| | | | | CCCTACCACGACTTCATCTTAGAAGAC |
| | | | | GCCGCATCACCCAAGTGCATTATGAAA |
| | | | | GAGAAAAAGAAGCCAGGAGAAACATTC |
| | | | | TTTATGTGCTCATGCTCCTCTGACGAA |
| | | | | TGCAACGACAACATTATCTTCTCTGAG |
| | | | | GAGTATAACACCTCAAATCCAGAC |
| Linker | 17 | DPGGGGSGGGGSNP | | |
| | | GS | | |
| Linker | 18 | GGGGSGGGGSGSDP | | |
| | | GS | | |
| Linker | 19 | DPGSGGGGSGGGGS | | |
| | | GS | | |
| Linker | 20 | GGGGSGGGGSGGGG | | |
| | | SDPGS | | |
| Linker | 21 | DPGSGGGGSGGGGS | | |
| | | GGGGS | | |
| Linker 7 | 22 | DPGSGSVPLGSGSN | | |
| | | PGS | | |
| Linker 8 | 23 | DPGSGGSVPLGSGG | | |
| | | SNPGS | | |
| Linker | 24 | DPGVLEREDKPTTS | | |
| | | KPNPGS | | |
| Linker | 25 | DPGVLEREDVPTTS | | |
| 10 | | YPNPGS | | |
| Linker | 26 | DPGVLEREDKVTTS | | |
| | | KYNPGS | | |
| Linker | 27 | DPVLEREDKVTTSK | | |
| 12 | | NPGS | | |
| Linker | 28 | DIEGRMD | | |
| Linker | 29 | GEGKSSGSGSESKA | | |
| | | S | | |
| Linker | 30 | GSTSGSGKPGSGEG | | |
| | | STKG | | |
| Linker | 31 | A(EAAAK)₄ALEA(EAAAK)₄A | | |
| Linker | 32 | (G4S)n, n = 1-10 | | |
| Linker | 33 | (Gly)n, n = 6-8 | | |
| Linker | 34 | (EAAAK)n, n = 1-6 | | |
| hM195 | 35 | DIQMTQSPSSLSAS | 72 | GACATTCAGATGACCCAGTCTCCGAGC |
| VL | | VGDRVTITCRASES | | TCTCTGTCCGCATCAGTAGGAGACAGG |
| | | VDNYGISFMNWFQQ | | GTCACCATCACATGCAGAGCCAGCGAA |
| | | KPGKAPKLLIYAAS | | AGTGTCGACAATTATGGCATTAGCTTT |
| | | NQGSGVPSRFSGSG | | ATGAACTGGTTCCAACAGAAACCCGGG |
| | | SGTDFTLTISSLQP | | AAGGCTCCTAAGCTTCTGATTTACGCT |
| | | DDFATYYCQQSKEV | | GCATCCAACCAAGGCTCCGGGGTACCC |
| | | PWTFGQGTKVEIK | | TCTCGCTTCTCAGGCAGTGGATCTGGG |
| | | | | ACAGACTTCACTCTCACCATTTCATCT |
| | | | | CTGCAGCCTGATGACTTCGCAACCTAT |
| | | | | TACTGTCAGCAAAGTAAGGAGGTTCCG |
| | | | | TGGACGTTCGGTCAAGGGACCAAGGTG |
| | | | | GAGATCAAA |
| hM195 | 36 | QVQLVQSGAEVKKP | 73 | CAGGTTCAGCTGGTGCAGTCTGGAGCT |
| VH | | GSSVKVSCKASGYT | | GAGGTGAAGAAGCCTGGGAGCTCAGTG |
| | | FTDYNMHWVRQAPG | | AAGGTTTCCTGCAAAGCTTCTGGCTAC |
| | | QGLEWIGYIYPYNG | | ACCTTCACTGACTACAACATGCACTGG |
| | | GTGYNQKFKSKATI | | GTGAGGCAGGCTCCTGGCCAAGGCCTG |
| | | TADESTNTAYMELS | | GAATGGATTGGATATATTTATCCTTAC |
| | | SLRSEDTAVYYCAR | | AATGGTGGTACCGGCTACAACCAGAAG |
| | | GRPAMDYWGQGTLV | | TTCAAGAGCAAGGCCACAATTACAGCA |
| | | TVSS | | GACGAGAGTACTAACACAGCCTACATG |
| | | | | GAACTCTCCAGCCTGAGGTCTGAGGAC |
| | | | | ACTGCAGTCTATTACTGCGCAAGAGGG |
| | | | | CGCCCCGCTATGGACTACTGGGGCCAA |
| | | | | GGGACTCTGGTCACTGTCTCTTCA |
| M2H12 | 37 | QVQLQQSGPELVRP | | |
| VH | | GTFVKISCKASGYT | | |
| | | FTNYDINWVNQRPG | | |
| | | QGLEWIGWIYPGDG | | |
| | | STKYNEKFKAKTAL | | |
| | | TADKSSSTAYLQLN | | |
| | | NLTSENSAVYFCAS | | |
| | | GYEDAMDYWGQGTS | | |
| | | VTVSS | | |
| M2H12 | 38 | DIKMTQSPSSMYAS | | |
| VL | | LGERVIINCKASQD | | |
| | | INSYLSWFQQKPGK | | |
| | | SPKTLIYRANRLVD | | |
| | | GVPSRFSGSGSGQD | | |
| | | YSLTISSLEYEDMG | | |
| | | IYYCLQYDEFPLTF | | |
| | | GAGTKLELKR | | |
| DRB2 | 39 | EVKLQESGPELVKP | | |
| VH | | GASVKMSCKASGYK | | |
| | | FTDYVVHWLKQKPG | | |
| | | QGLEWIGYINPYND | | |
| | | GTKYNEKFKGKATL | | |
| | | TSDKSSSTAYMEVS | | |
| | | SLTSEDSAVYYCAR | | |
| | | DYRYEVYGMDYWGQ | | |
| | | GYSVTVSS | | |
| DRB2 | 40 | DIVLTQSPTIMSAS | | |
| VL | | PGERVTMTCTASSS | | |
| | | VNYIH | | |
| | | WYQQKSGDSPLRWI | | |
| | | FDTSKVASGVPARF | | |
| | | SGSGSGTSYSLTIS | | |
| | | TMEAEDAATYYCQQ | | |
| | | WRSYPLTFGDGTRL | | |
| | | ELKRADAAPTVS | | |
| My9-6 | 41 | QVQLQQPGAEVVKP | | |
| VH | | GASVKMSCKASGYT | | |
| | | FTSYYIHWIKQTPG | | |
| | | QGLEWVGVIYPGND | | |
| | | DISYNQKFKGKATL | | |
| | | TADKSSTTAYMQLS | | |
| | | SLTSEDSAVYYCAR | | |
| | | EVRLRYFDVWGAGT | | |
| | | TVTVSS | | |
| My9-6 | 42 | NIMLTQSPSSLAVS | | |
| VL | | AGEKVTMSCKSSQS | | |
| | | VFFSSSQKNYLAWY | | |
| | | QQIPGQSPKLLIYW | | |
| | | ASTRESGVPDRFTG | | |
| | | SGSGTDFTLTISSV | | |
| | | QSEDLAIYYCHQYL | | |
| | | SSRTFGGGTKLEIK | | |
| | | R | | |
| MUC16- | 43 | DIELTQSPSSLAVS | 74 | GACATCGAGCTGACACAGAGCCCATCT |
| 1 VL | | AGEKVTMSCKSSQS | | AGCCTGGCTGTGTCTGCCGGCGAGAAA |
| | | LLNSRTRKNQLAWY | | GTGACCATGAGCTGCAAGAGCAGCCAG |
| | | QQKPGQSPELLIYW | | AGCCTGCTGAACAGCCGGACCAGAAAG |
| | | ASTRQSGVPDRFTG | | AATCAGCTGGCCTGGTATCAGCAGAAG |
| | | SGSGTDFTLTISSV | | CCCGGCCAATCTCCTGAGCTGCTGATC |
| | | QAEDLAVYYCQQSY | | TACTGGGCCAGCACAAGACAGAGCGGC |
| | | NLLTFGPGTKLEVK | | GTGCCCGATAGATTCACAGGATCTGGC |
| | | R | | AGCGGCACCGACTTCACCCTGACAATC |
| | | | | AGTTCTGTGCAGGCCGAGGACCTGGCC |
| | | | | GTGTACTACTGTCAGCAGAGCTACAAC |
| | | | | CTGCTGACCTTCGGACCCGGCACCAAG |
| | | | | CTGGAAGTGAAGAGA |
| MUC16- | 44 | VKLQESGGGFVKPG | 75 | GTGAAGCTGCAAGAGTCCGGCGGAGGC |
| 1 VH | | GSLKVSCAASGFTF | | TTTGTGAAGCCTGGCGGCTCTCTGAAA |
| | | SSYAMSWVRLSPEM | | GTGTCCTGTGCCGCCAGCGGCTTCACC |
| | | RLEWVATISSAGGY | | TTTAGCAGCTACGCCATGAGCTGGGTC |
| | | IFYSDSVQGRFTIS | | CGACTGAGCCCTGAGATGAGACTGGAA |
| | | RDNAKNTLHLQMGS | | TGGGTCGCCACCATCAGTAGCGCAGGC |
| | | LRSGDTAMYYCARQ | | GGCTACATCTTCTACAGCGACTCTGTG |
| | | GFGNYGDYYAMDYW | | CAGGGCAGATTCACCATCAGCCGGGAC |
| | | GQGTTVTVSS | | AACGCCAAGAACACCCTGCACCTCCAG |
| | | | | ATGGGCAGTCTGAGAAGCGGCGATACC |
| | | | | GCCATGTACTACTGCGCCAGACAAGGC |
| | | | | TTCGGCAACTACGGCGACTACTATGCC |
| | | | | ATGGATTACTGGGGCCAGGGCACCACC |
| | | | | GTGACAGTCTCTTCT |
| MUC16- | 45 | DIELTQSPSSLAVS | 76 | GACATCGAGCTGACACAGAGCCCATCT |
| 2 VL | | AGEKVTMSCKSSQS | | AGCCTGGCTGTGTCTGCCGGCGAGAAA |
| | | LLNSRTRKNQLAWY | | GTGACCATGAGCTGCAAGAGCAGCCAG |
| | | QQKTGQSPELLIYW | | AGCCTGCTGAACAGCCGGACCAGAAAG |
| | | ASTRQSGVPDRFTG | | AATCAGCTGGCCTGGTATCAGCAGAAA |
| | | SGSGTDFTLTISSV | | ACCGGACAGAGCCCCGAGCTGCTGATC |
| | | QAEDLAVYYCQQSY | | TACTGGGCCAGCACAAGACAGAGCGGC |
| | | NLLTFGPGTKLEIK | | GTGCCCGATAGATTCACAGGATCTGGC |
| | | R | | AGCGGCACCGACTTCACCCTGACAATC |
| | | | | AGTTCTGTGCAGGCCGAGGACCTGGCC |
| | | | | GTGTACTACTGTCAGCAGAGCTACAAC |
| | | | | CTGCTGACCTTCGGACCCGGCACCAAG |
| | | | | CTGGAAATCAAGAGA |
| MUC16- | 46 | VKLEESGGGFVKPG | 77 | GTGAAGCTGGAAGAGTCCGGCGGAGGC |
| 2 VH | | GSLKISCAASGFTF | | TTTGTGAAGCCTGGCGGAAGCCTGAAG |
| | | RNYAMSWVRLSPEM | | ATCAGCTGTGCCGCCAGCGGCTTCACC |
| | | RLEWVATISSAGGY | | TTCAGAAACTACGCCATGAGCTGGGTC |
| | | IFYSDSVQGRFTIS | | CGACTGAGCCCCGAGATGAGACTGGAA |
| | | RDNAKNTHLQMGSL | | TGGGTCGCCACAATCAGCAGCGCAGGC |
| | | RSGDTAMYYCARQG | | GGCTACATCTTCTACAGCGATAGCGTG |
| | | FGNYGDYYAMDYWG | | CAGGGCAGATTCACCATCAGCCGGGAC |
| | | QGTTVTVSS | | AACGCCAAGAACACCCTGCACCTCCAG |
| | | | | ATGGGCAGTCTGAGATCTGGCGACACC |
| | | | | GCCATGTACTACTGCGCCAGACAAGGC |
| | | | | TTCGGCAACTACGGCGACTACTATGCC |
| | | | | ATGGATTACTGGGGCCAGGGCACCACC |
| | | | | GTGACAGTCTCTTCT |
| MUC16- | 47 | DIKMAQSPSSVNAS | 78 | GACATCAAGATGGCTCAGTCCCCTTCT |
| 3 VL | | LGERVTITCKASRD | | AGCGTGAATGCTTCGCTAGGGGAGCGT |
| | | INNFLSWFHQKPGK | | GTGACCATCACATGTAAAGCATCACGC |
| | | SPKTLIYRANRLVD | | GACATAAATAATTTCCTTTCCTGGTTT |
| | | GVPSRFSGSGSGQD | | CATCAGAAACCGGGCAAGTCGCCTAAG |
| | | YSFTISSLEYEDVG | | ACGCTGATTTACAGAGCAAATCGGTTG |
| | | IYYCLQYGDLYTFG | | GTAGATGGAGTGCCAAGCAGATTCAGC |
| | | GGTKLEIK | | GGGAGCGGAAGTGGACAGGATTATAGC |
| | | | | TTCACTATTTCATCCCTGGAATACGAG |
| | | | | GACGTAGGTATCTATTATTGCCTCCAG |
| | | | | TATGGCGATCTTTACACATTTGGTGGG |
| | | | | GGGACTAAGCTGGAGATTAAG |
| MUC16- | 48 | DVQLLESGPGLVRP | 79 | GACGTGCAACTTCTGGAGAGCGGGCCA |
| 3 VH | | SQSLSLTCSVTGYS | | GGGCTAGTCAGGCCCTCCCAGTCGCTT |
| | | IVSHYYWNWIRQFP | | TCACTGACTTGCAGTGTGACCGGTTAC |
| | | GNKLEWMGYISSDG | | TCTATTGTGAGTCACTACTATTGGAAC |
| | | SNEYNPSLKNRISI | | TGGATTCGGCAGTTCCCAGGCAACAAA |
| | | SLDTSKNQFFLKFD | | CTGGAATGGATGGGGTACATATCTTCC |
| | | FVTTADTATYFCVR | | GATGGCTCGAATGAATATAACCCATCA |
| | | GVDYWGQGTTLTVS | | TTGAAAAATCGTATTTCCATCAGTCTG |
| | | S | | GATACGAGTAAAAACCAGTTTTTCCTC |
| | | | | AAATTCGATTTCGTGACTACAGCAGAT |
| | | | | ACTGCCACATACTTCTGTGTACGAGGT |
| | | | | GTCGATTATTGGGGACAGGGCACAACG |
| | | | | CTGACCGTAAGTTCT |
| MUC16- | 49 | DIQMTQSSSFLSVS | 80 | GACATCCAGATGACCCAGAGCAGCAGC |
| 4 VL | | LGGRVTITCKASDL | | TTCCTGAGCGTGTCCCTTGGCGGCAGA |
| | | IHNWLAWYQQKPGN | | GTGACCATCACCTGTAAAGCCAGCGAC |
| | | APRLLISGATSLET | | CTGATCCACAACTGGCTGGCCTGGTAT |
| | | GVPSRFSGSGSGND | | CAGCAGAAGCCTGGCAACGCTCCCAGA |
| | | YTLSIASLQTEDAA | | CTGCTGATTAGCGGCGCCACCTCTCTG |
| | | TYYCQQYWTTPFTF | | GAAACAGGCGTGCCAAGCAGATTTTCC |
| | | GSGTKLEIK | | GGCAGCGGCTCCGGCAACGACTACACA |
| | | | | CTGTCTATTGCCAGCCTGCAGACCGAG |
| | | | | GATGCCGCCACCTATTACTGCCAGCAG |
| | | | | TACTGGACCACACCTTTCACCTTTGGC |
| | | | | AGCGGCACCAAGCTGGAAATCAAG |
| MUC16- | 50 | DVQLQESGPGLVNP | 81 | GACGTTCAGCTGCAAGAGTCTGGCCCT |
| 4 VH | | SQSLSLTCTVTGYS | | GGCCTGGTCAATCCTAGCCAGAGCCTG |
| | | ITNDYAWNWIRQFP | | AGCCTGACATGTACCGTGACCGGCTAC |
| | | GNKLEWMGYINYSG | | AGCATCACCAACGACTACGCCTGGAAC |
| | | YTTYNPSLKSRISI | | TGGATCAGACAGTTCCCCGGCAACAAG |
| | | TRDTSKNQFFLHLN | | CTGGAATGGATGGGCTACATCAACTAC |
| | | SVTTEDTATYYCAR | | AGCGGCTACACCACCTACAATCCCAGC |
| | | WDGGLTYWGQGTLV | | CTGAAGTCCCGGATCTCCATCACCAGA |
| | | TVSA | | GACACCAGCAAGAACCAGTTCTTCCTG |
| | | | | CACCTGAACAGCGTGACCACCGAGGAT |
| | | | | ACCGCCACCTACTACTGCGCTAGATGG |
| | | | | GATGGCGGCCTGACATATTGGGGCCAG |
| | | | | GGAACACTGGTCACCGTGTCTGCT |
| MUC16- | 51 | DIQMTQSPSSLSAS | 82 | GACATCCAGATGACCCAGAGCCCCAGC |
| 5 VL | | VGDRVTITCKASDL | | AGCCTGAGCGCCAGCGTGGGCGACAGG |
| | | IHNWLAWYQQKPGK | | GTGACCATCACCTGCAAGGCCAGCGAC |
| | | APKLLISGATSLET | | CTGATCCACAACTGGCTGGCCTGGTAC |
| | | GVPSRFSGSGSGTD | | CAGCAGAAGCCCGGCAAGGCCCCCAAG |
| | | FTLTISSLQPEDFA | | CTGCTGATCAGCGGCGCCACCAGCCTG |
| | | TYYCQQYWTTPFTF | | GAGACCGGCGTGCCCAGCAGGTTCAGC |
| | | GQGTKVEIKR | | GGCAGCGGCAGCGGCACCGACTTCACC |
| | | | | CTGACCATCAGCAGCCTGCAGCCCGAG |
| | | | | GACTTCGCCACCTACTACTGCCAGCAG |
| | | | | TACTGGACCACCCCCTTCACCTTCGGC |
| | | | | CAGGGCACCAAGGTGGAGATCAAGAGG |
| MUC16- | 52 | EVQLVESGGGLVQP | 83 | GAGGTGCAGCTGGTGGAGAGCGGCGGC |
| 5 VH-L | | GGSLRLSCAASGYS | | GGCCTGGTGCAGCCCGGCGGCAGCCTG |
| | | ITNDYAWNWVRQAP | | AGGCTGAGCTGCGCCGCCAGCGGCTAC |
| | | GKGLEWVGYINYSG | | AGCATCACCAACGACTACGCCTGGAAC |
| | | YTTYNPSLKSRFTI | | TGGGTGAGGCAGGCCCCCGGCAAGGGC |
| | | SRDNSKNTLYLQMN | | CTGGAGTGGGTGGGCTACATCAACTAC |
| | | SLRAEDTAVYYCAR | | AGCGGCTACACCACCTACAACCCCAGC |
| | | WDGGLTYWGQGTLV | | CTGAAGAGCAGGTTCACCATCAGCAGG |
| | | TVSS | | GACAACAGCAAGAACACCCTGTACCTG |
| | | | | CAGATGAACAGCCTGAGGGCCGAGGAC |
| | | | | ACCGCCGTGTACTACTGCGCCAGGTGG |
| | | | | GACGGCGGCCTGACCTACTGGGGCCAG |
| | | | | GGCACCCTGGTGACCGTGAGCAGC |
| MUC16- | 53 | EVQLVESGGGLVQP | 84 | GAGGTGCAGCTGGTGGAGAGCGGCGGC |
| 5 VH-F | | GGSLRLSCAASGYS | | GGCCTGGTGCAGCCCGGCGGCAGCCTG |
| | | ITNDYAWNWVRQAP | | AGGCTGAGCTGCGCCGCCAGCGGCTAC |
| | | GKGLEWVGYINYSG | | AGCATCACCAACGACTACGCCTGGAAC |
| | | YTTYNPSLKSRFTI | | TGGGTGAGGCAGGCCCCCGGCAAGGGC |
| | | SRDNSKNTFYLQMN | | CTGGAGTGGGTGGGCTACATCAACTAC |
| | | SLRAEDTAVYYCAR | | AGCGGCTACACCACCTACAACCCCAGC |
| | | WDGGLTYWGQGTLV | | CTGAAGAGCAGGTTCACCATCAGCAGG |
| | | TVSS | | GACAACAGCAAGAACACCTTCTACCTG |
| | | | | CAGATGAACAGCCTGAGGGCCGAGGAC |
| | | | | ACCGCCGTGTACTACTGCGCCAGGTGG |
| | | | | GACGGCGGCCTGACCTACTGGGGCCAG |
| | | | | GGCACCCTGGTGACCGTGAGCAGC |
| MUC16- | 54 | DIVLTQSPAIMSAS | 85 | GACATCGTGCTGACACAGAGCCCTGCC |
| 6 VL | | LGERVTMTCTASSS | | ATCATGTCTGCCAGCCTCGGCGAGCGA |
| | | VSSSYLHWYQQKPG | | GTGACCATGACATGTACAGCCAGCAGC |
| | | SSPKLWIYSTSNLA | | AGCGTGTCCAGCAGCTACCTGCATTGG |
| | | SGVPGRFSGSGSGT | | TATCAGCAGAAGCCCGGCAGCAGCCCC |
| | | SYSLTISSMEAEDA | | AAGCTGTGGATCTACAGCACAAGCAAT |
| | | ATYYCHQYHRSPYT | | CTGGCCAGCGGCGTGCCAGGCAGATTT |
| | | FGGGTKVEIKR | | TCTGGTTCTGGCAGCGGCACCAGCTAC |
| | | | | AGCCTGACAATCAGCAGCATGGAAGCC |
| | | | | GAGGATGCCGCCACCTACTACTGCCAC |
| | | | | CAGTACCACAGAAGCCCCTACACCTTT |
| | | | | GGCGGAGGCACCAAGGTGGAAATCAAG |
| | | | | CGG |
| MUC16- | 55 | DIQMTQSPSSLSAS | 86 | GACATCCAGATGACACAGAGCCCTAGC |
| 7 VL | | VGDRVTITCTASSS | | AGCCTGTCTGCCAGCGTGGGAGACAGA |
| | | VSSSYLHWYQQKPG | | GTGACCATCACCTGTACAGCCAGCAGC |
| | | KAPKLLIYSTSNLA | | AGCGTGTCCAGCAGCTACCTGCATTGG |
| | | SGVPSRFSGSGSGT | | TATCAGCAGAAGCCCGGCAAGGCCCCT |
| | | DFTLTISSLQPEDF | | AAGCTGCTGATCTACAGCACCAGCAAT |
| | | ATYYCHQYHRSPYT | | CTGGCCAGCGGCGTGCCAAGCAGATTT |
| | | FGQGTKVEIKR | | TCTGGCTCTGGCAGCGGCACCGACTTC |
| | | | | ACCCTGACCATATCTAGCCTGCAGCCT |
| | | | | GAGGACTTCGCCACCTACTACTGCCAC |
| | | | | CAGTACCACAGAAGCCCCTACACCTTT |
| | | | | GGCCAGGGCACCAAGGTGGAAATCAAG |
| | | | | CGG |
| MUC16- | 56 | EVQLVESGGGLVQP | 87 | GAGGTGCAGCTGGTTGAATCTGGCGGA |
| 7 VH | | GGSLRLSCAASGFN | | GGACTGGTTCAGCCTGGCGGATCTCTG |
| | | IKDTYMHWVRQAPG | | AGACTGTCTTGTGCCGCCAGCGGCTTC |
| | | KGLEWVGRVDPANG | | AACATCAAGGACACCTACATGCACTGG |
| | | NTKYDPKFQGRFTI | | GTCCGACAGGCCCCTGGCAAAGGACTT |
| | | SADTSKNTAYLQMN | | GAGTGGGTTGGAAGAGTGGACCCCGCC |
| | | SLRAEDTAVYYCVR | | AACGGCAACACCAAATACGACCCCAAG |
| | | DYYGHTYGFAFWGQ | | TTCCAGGGCAGATTCACCATCAGCGCC |
| | | GTLVTVSS | | GACACCAGCAAGAACACCGCCTACCTG |
| | | | | CAGATGAACAGCCTGAGAGCCGAGGAC |
| | | | | ACCGCCGTGTACTATTGCGTGCGGGAT |
| | | | | TACTACGGCCATACCTACGGCTTCGCC |
| | | | | TTTTGGGGCCAGGGCACACTGGTTACC |
| | | | | GTTAGCTCT |
| CD8alpha | 57 | KPTTTPAPRPPTPA | 88 | AAGCCCACCACCACCCCTGCCCCTAGA |
| hinge | | PTIASQPLSLRPEA | | CCTCCAACCCCAGCCCCTACAATCGCC |
| | | CRPAAGGAVHTRGL | | AGCCAGCCCCTGAGCCTGAGGCCCGAA |
| | | DFACD | | GCCTGTAGACCTGCCGCTGGCGGAGCC |
| | | | | GTGCACACCAGAGGCCTGGATTTCGCC |
| | | | | TGCGAC |
| CD8alpha | 58 | KPTTTPAPRPPTPA | 89 | AAACCTACTACAACTCCTGCCCCCCGG |
| 2x | | PTIASQPLSLRPEA | | CCTCCTACACCAGCTCCTACTATCGCC |
| | | SRPAAGGAVHTRGL | | TCCCAGCCACTCAGTCTCAGACCCGAG |
| | | DFASDKPTTTPAPR | | GCTTCTAGGCCAGCGGCCGGAGGCGCG |
| | | PPTPAPTIASQPLS | | GTCCACACCCGCGGGCTGGACTTTGCA |
| | | LRPEACRPAAGGAV | | TCCGATAAGCCCACCACCACCCCTGCC |
| | | HTRGLDFACD | | CCTAGACCTCCAACCCCAGCCCCTACA |
| | | | | ATCGCCAGCCAGCCCCTGAGCCTGAGG |
| | | | | CCCGAAGCCTGTAGACCTGCCGCTGGC |
| | | | | GGAGCCGTGCACACCAGAGGCCTGGAT |
| | | | | TTCGCCTGCGAC |
| CD8alpha | 59 | KPTTTPAPRPPTPA | 90 | AAGCCTACCACCACCCCCGCACCTCGT |
| 3x | | PTIASQPLSLRPEA | | CCTCCAACCCCTGCACCTACGATTGCC |
| | | SRPAAGGAVHTRGL | | AGTCAGCCTCTTTCACTGCGGCCTGAG |
| | | DFASDKPTTTPAPR | | GCCAGCAGACCAGCTGCCGGCGGTGCC |
| | | PPTPAPTIASQPLS | | GTCCATACAAGAGGACTGGACTTCGCG |
| | | LRPEASRPAAGGAV | | TCCGATAAACCTACTACCACTCCAGCC |
| | | HTRGLDFASDKPTT | | CCAAGGCCCCCAACCCCAGCACCGACT |
| | | TPAPRPPTPAPTIA | | ATCGCATCACAGCCTTTGTCACTGCGT |
| | | SQPLSLRPEACRPA | | CCTGAAGCCAGCCGGCCAGCTGCAGGG |
| | | AGGAVHTRGLDFAC | | GGGGCCGTCCACACAAGGGGACTCGAC |
| | | D | | TTTGCGAGTGATAAGCCCACCACCACC |
| | | | | CCTGCCCCTAGACCTCCAACCCCAGCC |
| | | | | CCTACAATCGCCAGCCAGCCCCTGAGC |
| | | | | CTGAGGCCCGAAGCCTGTAGACCTGCC |
| | | | | GCTGGCGGAGCCGTGCACACCAGAGGC |
| | | | | CTGGATTTCGCCTGCGAC |
| CD8alpa | 60 | TTPAPRPPTPAPTI | 91 | AAGCCTACCACCACCCCCGCACCTCGT |
| 4x | | ASQPLSLRPEASRP | | CCTCCAACCCCTGCACCTACGATTGCC |
| | | AAGGAVHTRGLDFA | | AGTCAGCCTCTTTCACTGCGGCCTGAG |
| | | SDKPTTTPAPRPPT | | GCCAGCAGACCAGCTGCCGGCGGTGCC |
| | | PAPTIASQPLSLRP | | GTCCATACAAGAGGACTGGACTTCGCG |
| | | EASRPAAGGAVHTR | | TCCGATAAACCTACTACCACTCCAGCC |
| | | GLDFASDKPTTTPA | | CCAAGGCCCCCAACCCCAGCACCGACT |
| | | PRPPTPAPTIASQP | | ATCGCATCACAGCCTTTGTCACTGCGT |
| | | LSLRPEASRPAAGG | | CCTGAAGCCAGCCGGCCAGCTGCAGGG |
| | | AVHTRGLDFASDKP | | GGGGCCGTCCACACAAGGGGACTCGAC |
| | | TTTPAPRPPTPAPT | | TTTGCGAGTGATAAACCTACTACAACT |
| | | IASQPLSLRPEACR | | CCTGCCCCCCGGCCTCCTACACCAGCT |
| | | PAAGGAVHTRGLDF | | CCTACTATCGCCTCCCAGCCACTCAGT |
| | | ACD | | CTCAGACCCGAGGCTTCTAGGCCAGCG |
| | | | | GCCGGAGGCGCGGTCCACACCCGCGGG |
| | | | | CTGGACTTTGCATCCGATAAGCCCACC |
| | | | | ACCACCCCTGCCCCTAGACCTCCAACC |
| | | | | CCAGCCCCTACAATCGCCAGCCAGCCC |
| | | | | CTGAGCCTGAGGCCCGAAGCCTGTAGA |
| | | | | CCTGCCGCTGGCGGAGCCGTGCACACC |
| | | | | AGAGGCCTGGATTTCGCCTGCGAC |
| CD8alpha | 61 | IYIWAPLAGTCGVL | 92 | ATCTACATCTGGGCCCCTCTGGCCGGC |
| TM | | LLSLVITLYCNHRN | | ACCTGTGGCGTGCTGCTGCTGAGCCTG |
| | | | | GTCATCACCCTGTACTGCAACCACCGG |
| | | | | AAT |
| CD28 | 62 | FWVLVVVGGVLACY | 93 | TTTTGGGTGCTGGTGGTGGTTGGTGGA |
| TM | | SLLVTVAFIIFWV | | GTCCTGGCTTGCTATAGCTTGCTAGTA |
| | | | | ACAGTGGCCTTTATTATTTTCTGGGTG |
| 4-1BB | 63 | KRGRKKLLYIFKQP | 94 | AAGAGAGGCCGGAAGAAACTGCTGTAC |
| signaling | | FMRPVQTTQEEDGC | | ATCTTCAAGCAGCCCTTCATGCGGCCC |
| domain | | SCRFPEEEEGGCEL | | GTGCAGACCACCCAGGAAGAGGACGGC |
| | | | | TGCAGCTGCCGGTTCCCCGAGGAAGAG |
| | | | | GAAGGCGGCTGCGAACTG |
| CD28 | 64 | RSKRSRGGHSDYMN | 95 | AGGAGCAAGCGGAGCAGAGGCGGCCAC |
| signaling | | MTPRRPGPTRKHYQ | | AGCGACTACATGAACATGACCCCCCGG |
| domain | | PYAPPRDFAAYRS | | AGGCCTGGCCCCACCCGGAAGCACTAC |
| | | | | CAGCCCTACGCCCCTCCCAGGGACTTC |
| | | | | GCCGCCTACCGGAGC |
| DNAX- | 65 | LCARPRRSPAQEDG | 96 | CTGTGCGCACGCCCACGCCGCAGCCCC |
| activation | | KVYINMPGRG | | GCCCAAGAAGATGGCAAAGTCTACATC |
| protein | | | | AACATGCCAGGCAGGGGC |
| 10 (DAP | | | | |
| 10) | | | | |
| Signaling | | | | |
| Domain | | | | |
| DNAX- | 66 | YFLGRLVPRGRGAA | 97 | TACTTCCTGGGCCGGCTGGTCCCTCGG |
| activation | | EAATRKQRITETES | | GGGCGAGGGGCTGCGGAGGCAGCGACC |
| protein | | PYQELQGQRSDVYS | | CGGAAACAGCGTATCACTGAGACCGAG |
| 12 | | DLNTQRPYYK | | TCGCCTTATCAGGAGCTCCAGGGTCAG |
| (DAP12) | | | | AGGTCGGATGTCTACAGCGACCTCAAC |
| Signaling | | | | ACACAGAGGCCGTATTACAAA |
| Domain | | | | |
| CD3ζ | 67 | RVKFSRSADAPAYQ | 98 | CGGGTGAAGTTCAGCCGGAGCGCCGAC |
| signaling | | QGQNQLYNELNLGR | | GCCCCTGCCTACCAGCAGGGCCAGAAC |
| domain | | REEYDVLDKRRGRD | | CAGCTGTACAACGAGCTGAACCTGGGC |
| | | PEMGGKPRRKNPQE | | CGGAGGGAGGAGTACGACGTGCTGGAC |
| | | GLYNELQKDKMAEA | | AAGCGGAGAGGCCGGGACCCTGAGATG |
| | | YSEIGMKGERRRGK | | GGCGGCAAGCCCCGGAGAAAGAACCCT |
| | | GHDGLYQGLSTATK | | CAGGAGGGCCTGTATAACGAACTGCAG |
| | | DTYDALHMQALPPR | | AAAGACAAGATGGCCGAGGCCTACAGC |
| | | | | GAGATCGGCATGAAGGGCGAGCGGCGG |
| | | | | AGGGGCAAGGGCCACGACGGCCTGTAC |
| | | | | CAGGGCCTGAGCACCGCCACCAAGGAT |
| | | | | ACCTACGACGCCCTGCACATGCAGGCC |
| | | | | CTGCCCCCCAGA |
| HER1t | 68 | RKVCNGIGIGEFKD | 99 | CGCAAAGTGTGTAACGGAATAGGTATT |
| | | SLSINATNIKHFKN | | GGTGAATTTAAAGACTCACTCTCCATA |
| | | CTSISGDLHILPVA | | AATGCTACGAATATTAAACACTTCAAA |
| | | FRGDSFTHTPPLDP | | AACTGCACCTCCATCAGTGGCGATCTC |
| | | QELDILKTVKEITG | | CACATCCTGCCGGTGGCATTTAGGGGT |
| | | FLLIQAWPENRTDL | | GACTCCTTCACACATACTCCTCCTCTG |
| | | HAFENLEIIRGRTK | | GATCCACAGGAACTGGATATTCTGAAA |
| | | QHGQFSLAVVSLNI | | ACCGTAAAGGAAATCACAGGGTTTTTG |
| | | TSLGLRSLKEISDG | | CTGATTCAGGCTTGGCCTGAAAACAGG |
| | | DVIISGNKNLCYAN | | ACGGACCTCCATGCCTTTGAGAACCTA |
| | | TINWKKLFGTSGQK | | GAAATCATACGCGGCAGGACCAAGCAA |
| | | TKIISNRGENSCKA | | CATGGTCAGTTTTCTCTTGCAGTCGTC |
| | | TGQVCHALCSPEGC | | AGCCTGAACATAACATCCTTGGGATTA |
| | | WGPEPRDCVSCRNV | | CGCTCCCTCAAGGAGATAAGTGATGGA |
| | | SRGRECVDKCNLLE | | GATGTGATAATTTCAGGAAACAAAAAT |
| | | GEPREFVENSECIQ | | TTGTGCTATGCAAATACAATAAACTGG |
| | | CHPECLPQAMNITC | | AAAAAACTGTTTGGGACCTCCGGTCAG |
| | | TGRGPDNCIQCAHY | | AAAACCAAAATTATAAGCAACAGAGGT |
| | | IDGPHCVKTCPAGV | | GAAAACAGCTGCAAGGCCACAGGCCAG |
| | | MGENNTLVWKYADA | | GTCTGCCATGCCTTGTGCTCCCCCGAG |
| | | GHVCHLCHPNCTYG | | GGCTGCTGGGGCCCGGAGCCCAGGGAC |
| | | CTGPGLEGCPTNGP | | TGCGTCTCTTGCCGGAATGTCAGCCGA |
| | | KIPSIATGMVGALL | | GGCAGGGAATGCGTGGACAAGTGCAAC |
| | | LLLVVALGIGLFM | | CTTCTGGAGGGTGAGCCAAGGGAGTTT |
| | | | | GTGGAGAACTCTGAGTGCATACAGTGC |
| | | | | CACCCAGAGTGCCTGCCTCAGGCCATG |
| | | | | AACATCACCTGCACAGGACGGGGACCA |
| | | | | GACAACTGTATCCAGTGTGCCCACTAC |
| | | | | ATTGACGGCCCCCACTGCGTCAAGACC |
| | | | | TGCCCGGCAGGAGTCATGGGAGAAAAC |
| | | | | AACACCCTGGTCTGGAAGTACGCAGAC |
| | | | | GCCGGCCATGTGTGCCACCTGTGCCAT |
| | | | | CCAAACTGCACCTACGGATGCACTGGG |
| | | | | CCAGGTCTTGAAGGCTGTCCAACGAAT |
| | | | | GGGCCTAAGATCCCGTCCATCGCCACT |
| | | | | GGGATGGTGGGGGCCCTCCTCTTGCTG |
| | | | | CTGGTGGTGGCCCTGGGGATCGGCCTC |
| | | | | TTCATG |
| HER1t- | 69 | RKVCNGIGIGEFKD | 100 | CGCAAAGTGTGTAACGGAATAGGTATT |
| 1 | | SLSINATNIKHFKN | | GGTGAATTTAAAGACTCACTCTCCATA |
| | | CTSISGDLHILPVA | | AATGCTACGAATATTAAACACTTCAAA |
| | | FRGDSFTHTPPLDP | | AACTGCACCTCCATCAGTGGCGATCTC |
| | | QELDILKTVKEITG | | CACATCCTGCCGGTGGCATTTAGGGGT |
| | | FLLIQAWPENRTDL | | GACTCCTTCACACATACTCCTCCTCTG |
| | | HAFENLEIIRGRTK | | GATCCACAGGAACTGGATATTCTGAAA |
| | | QHGQFSLAVVSLNI | | ACCGTAAAGGAAATCACAGGGTTTTTG |
| | | TSLGLRSLKEISDG | | CTGATTCAGGCTTGGCCTGAAAACAGG |
| | | DVIISGNKNLCYAN | | ACGGACCTCCATGCCTTTGAGAACCTA |
| | | TINWKKLFGTSGQK | | GAAATCATACGCGGCAGGACCAAGCAA |
| | | TKIISNRGENSCKA | | CATGGTCAGTTTTCTCTTGCAGTCGTC |
| | | TGQVCHALCSPEGC | | AGCCTGAACATAACATCCTTGGGATTA |
| | | WGPEPRDCVSGGGG | | CGCTCCCTCAAGGAGATAAGTGATGGA |
| | | SGGGSGGGGSGGGG | | GATGTGATAATTTCAGGAAACAAAAAT |
| | | SFWVLVVVGGVLAC | | TTGTGCTATGCAAATACAATAAACTGG |
| | | YSLLVTVAFIIFWV | | AAAAAACTGTTTGGGACCTCCGGTCAG |
| | | RSKRS | | AAAACCAAAATTATAAGCAACAGAGGT |
| | | | | GAAAACAGCTGCAAGGCCACAGGCCAG |
| | | | | GTCTGCCATGCCTTGTGCTCCCCCGAG |
| | | | | GGCTGCTGGGGCCCGGAGCCCAGGGAC |
| | | | | TGCGTCTCTGGTGGCGGTGGCTCGGGC |
| | | | | GGTGGTGGGTCGGGTGGCGGCGGATCT |
| | | | | GGTGGCGGTGGCTCGTTTTGGGTGCTG |
| | | | | GTGGTGGTTGGTGGAGTCCTGGCTTGC |
| | | | | TATAGCTTGCTAGTAACAGTGGCCTTT |
| | | | | ATTATTTTCTGGGTGAGGAGTAAGAGG |
| | | | | AGC |
| FL | 70 | MTTPRNSVNGTFPA | 101 | ATGACAACACCCAGAAATTCAGTAAAT |
| CD20 | | EPMKGPIAMQSGPK | | GGGACTTTCCCGGCAGAGCCAATGAAA |
| | | PLFRRMSSLVGPTQ | | GGCCCTATTGCTATGCAATCTGGTCCA |
| | | SFFMRESKTLGAVQ | | AAACCACTCTTCAGGAGGATGTCTTCA |
| | | IMNGLFHIALGGLL | | CTGGTGGGCCCCACGCAAAGCTTCTTC |
| | | MIPAGIYAPICVTV | | ATGAGGGAATCTAAGACTTTGGGGGCT |
| | | WYPLWGGIMYIISG | | GTCCAGATTATGAATGGGCTCTTCCAC |
| | | SLLAATEKNSRKCL | | ATTGCCCTGGGGGGTCTTCTGATGATC |
| | | VKGKMIMNSLSLFA | | CCAGCAGGGATCTATGCACCCATCTGT |
| | | AISGMILSIMDILN | | GTGACTGTGTGGTACCCTCTCTGGGGA |
| | | IKISHFLKMESLNF | | GGCATTATGTATATTATTTCCGGATCA |
| | | IRAHTPYINIYNCE | | CTCCTGGCAGCAACGGAGAAAAACTCC |
| | | PANPSEKNSPSTQY | | AGGAAGTGTTTGGTCAAAGGAAAAATG |
| | | CYSIQSLFLGILSV | | ATAATGAATTCATTGAGCCTCTTTGCT |
| | | MLIFAFFQELVIAG | | GCCATTTCTGGAATGATTCTTTCAATC |
| | | IVENEWKRTCSRPK | | ATGGACATACTTAATATTAAAATTTCC |
| | | SNIVLLSAEEKKEQ | | CATTTTTTAAAAATGGAGAGTCTGAAT |
| | | TIEIKEEVVGLTET | | TTTATTAGAGCTCACACACCATATATT |
| | | SSQPKNEEDIEIIP | | AACATATACAACTGTGAACCAGCTAAT |
| | | IQEEEEEETETNFP | | CCCTCTGAGAAAAACTCCCCATCTACC |
| | | EPPQDQESSPIEND | | CAATACTGTTACAGCATACAATCTCTG |
| | | SSP | | TTCTTGGGCATTTTGTCAGTGATGCTG |
| | | | | ATCTTTGCCTTCTTCCAGGAACTTGTA |
| | | | | ATAGCTGGCATCGTTGAGAATGAATGG |
| | | | | AAAAGAACGTGCTCCAGACCCAAATCT |
| | | | | AACATAGTTCTCCTGTCAGCAGAAGAA |
| | | | | AAAAAAGAACAGACTATTGAAATAAAA |
| | | | | GAAGAAGTGGTTGGGCTAACTGAAACA |
| | | | | TCTTCCCAACCAAAGAATGAAGAAGAC |
| | | | | ATTGAAATTATTCCAATCCAAGAAGAG |
| | | | | GAAGAAGAAGAAACAGAGACGAACTTT |
| | | | | CCAGAACCTCCCCAAGATCAGGAATCC |
| | | | | TCACCAATAGAAAATGACAGCTCTCCT |
| CD20t-1 | 71 | MTTPRNSVNGTFPA | 102 | ATGACCACACCACGGAACTCTGTGAAT |
| | | EPMKGPIAMQSGPK | | GGCACCTTCCCAGCAGAGCCAATGAAG |
| | | PLFRRMSSLVGPTQ | | GGACCAATCGCAATGCAGAGCGGACCC |
| | | SFFMRESKTLGAVQ | | AAGCCTCTGTTTCGGAGAATGAGCTCC |
| | | IMNGLFHIALGGLL | | CTGGTGGGCCCAACCCAGTCCTTCTTT |
| | | MIPAGIYAPICVTV | | ATGAGAGAGTCTAAGACACTGGGCGCC |
| | | WYPLWGGIMYIISG | | GTGCAGATCATGAACGGACTGTTCCAC |
| | | SLLAATEKNSRKCL | | ATCGCCCTGGGAGGACTGCTGATGATC |
| | | VKGKMIMNSLSLFA | | CCAGCCGGCATCTACGCCCCTATCTGC |
| | | AISGMILSIMDILN | | GTGACCGTGTGGTACCCTCTGTGGGGC |
| | | IKISHFLKMESLNF | | GGCATCATGTATATCATCTCCGGCTCT |
| | | IRAHTPYINIYNCE | | CTGCTGGCCGCCACAGAGAAGAACAGC |
| | | PANPSEKNSPSTQY | | AGGAAGTGTCTGGTGAAGGGCAAGATG |
| | | CYSIQSLFLGILSV | | ATCATGAATAGCCTGTCCCTGTTTGCC |
| | | MLIFAFFQELVIAG | | GCCATCTCTGGCATGATCCTGAGCATC |
| | | IVENEWKRTCSRPK | | ATGGACATCCTGAACATCAAGATCAGC |
| | | SNIVLLSAEEKKEQ | | CACTTCCTGAAGATGGAGAGCCTGAAC |
| | | TIEIKEEVVGLTET | | TTCATCAGAGCCCACACCCCTTACATC |
| | | SSQPKNEEDIE | | AACATCTATAATTGCGAGCCTGCCAAC |
| | | | | CCATCCGAGAAGAATTCTCCAAGCACA |
| | | | | CAGTACTGTTATTCCATCCAGTCTCTG |
| | | | | TTCCTGGGCATCCTGTCTGTGATGCTG |
| | | | | ATCTTTGCCTTCTTTCAGGAGCTGGTC |
| | | | | ATCGCCGGCATCGTGGAGAACGAGTGG |
| | | | | AAGAGGACCTGCAGCCGCCCCAAGTCC |
| | | | | AATATCGTGCTGCTGTCCGCCGAGGAG |
| | | | | AAGAAGGAGCAGACAATCGAGATCAAG |
| | | | | GAGGAGGTGGTGGGCCTGACCGAGACA |
| | | | | TCTAGCCAGCCTAAGAATGAGGAGGAT |
| | | | | ATCGAG |
| mbIL15 | 103 | MDWTWILFLVAAAT | 106 | ATGGATTGGACCTGGATTCTGTTTCTG |
| | | RVHSNWVNVISDLK | | GTGGCCGCTGCCACAAGAGTGCACAGC |
| | | KIEDLIQSMHIDAT | | AACTGGGTGAATGTGATCAGCGACCTG |
| | | LYTESDVHPSCKVT | | AAGAAGATCGAGGATCTGATCCAGAGC |
| | | AMKCFLLELQVISL | | ATGCACATTGATGCCACCCTGTACACA |
| | | ESGDASIHDTVENL | | GAATCTGATGTGCACCCTAGCTGTAAA |
| | | IILANNSLSSNGNV | | GTGACCGCCATGAAGTGTTTTCTGCTG |
| | | TESGCKECEELEEK | | GAGCTGCAGGTGATTTCTCTGGAAAGC |
| | | NIKEFLQSFVHIVQ | | GGAGATGCCTCTATCCACGACACAGTG |
| | | MFINTSSGGGSGGG | | GAGAATCTGATCATCCTGGCCAACAAT |
| | | GSGGGGSGGGGSGG | | AGCCTGAGCAGCAATGGCAATGTGACA |
| | | GSLQITCPPPMSVE | | GAGTCTGGCTGTAAGGAGTGTGAGGAG |
| | | HADIWVKSYSLYSR | | CTGGAGGAGAAGAACATCAAGGAGTTT |
| | | ERYICNSGFKRKAG | | CTGCAGAGCTTTGTGCACATCGTGCAG |
| | | TSSLTECVLNKATN | | ATGTTCATCAATACAAGCTCTGGCGGA |
| | | VAHWTTPSLKCIRD | | GGATCTGGAGGAGGCGGATCTGGAGGA |
| | | PALVHQRPAPPSTV | | GGAGGCAGTGGAGGCGGAGGATCTGGC |
| | | TTAGVTPQPESLSP | | GGAGGATCTCTGCAGATTACATGCCCT |
| | | SGKEPAASSPSSNN | | CCTCCAATGTCTGTGGAGCACGCCGAT |
| | | TAATTAAIVPGSQL | | ATTTGGGTGAAGTCCTACAGCCTGTAC |
| | | MPSKSPSTGTTEIS | | AGCAGAGAGAGATACATCTGCAACAGC |
| | | SHESSHGTPSQTTA | | GGCTTTAAGAGAAAGGCCGGCACCTCT |
| | | KNWELTASASHQPP | | TCTCTGACAGAGTGCGTGCTGAATAAG |
| | | GVYPQGHSDTTVAI | | GCCACAAATGTGGCCCACTGGACAACA |
| | | STSTVLLCGLSAVS | | CCTAGCCTGAAGTGCATTAGAGATCCT |
| | | LLACYLKSRQTPPL | | GCCCTGGTCCACCAGAGGCCTGCCCCT |
| | | ASVEMEAMEALPVT | | CCATCTACAGTGACAACAGCCGGAGTG |
| | | WGTSSRDEDLENCE | | ACACCTCAGCCTGAATCTCTGAGCCCT |
| | | HHL | | TCTGGAAAAGAACCTGCCGCCAGCTCT |
| | | | | CCTAGCTCTAATAATACCGCCGCCACA |
| | | | | ACAGCCGCCATTGTGCCTGGATCTCAG |
| | | | | CTGATGCCTAGCAAGTCTCCTAGCACA |
| | | | | GGCACAACAGAGATCAGCAGCCACGAA |
| | | | | TCTTCTCACGGAACACCTTCTCAGACC |
| | | | | ACCGCCAAGAATTGGGAGCTGACAGCC |
| | | | | TCTGCCTCTCACCAGCCTCCAGGAGTG |
| | | | | TATCCTCAGGGCCACTCTGATACAACA |
| | | | | GTGGCCATCAGCACATCTACAGTGCTG |
| | | | | CTGTGTGGACTGTCTGCCGTGTCTCTG |
| | | | | CTGGCCTGTTACCTGAAGTCTAGACAG |
| | | | | ACACCTCCTCTGGCCTCTGTGGAGATG |
| | | | | GAGGCCATGGAAGCCCTGCCTGTGACA |
| | | | | TGGGGAACAAGCAGCAGAGATGAGGAC |
| | | | | CTGGAGAATTGTTCTCACCACCTG |
| IL-15 | 104 | NWVNVISDLKKIED | 107 | AACTGGGTGAATGTGATCAGCGACCTG |
| | | LIQSMHIDATLYTE | | AAGAAGATCGAGGATCTGATCCAGAGC |
| | | SDVHPSCKVTAMKC | | ATGCACATTGATGCCACCCTGTACACA |
| | | FLLELQVISLESGD | | GAATCTGATGTGCACCCTAGCTGTAAA |
| | | ASIHDTVENLIILA | | GTGACCGCCATGAAGTGTTTTCTGCTG |
| | | NNSLSSNGNVTESG | | GAGCTGCAGGTGATTTCTCTGGAAAGC |
| | | CKECEELEEKNIKE | | GGAGATGCCTCTATCCACGACACAGTG |
| | | FLQSFVHIVQMFIN | | GAGAATCTGATCATCCTGGCCAACAAT |
| | | TS | | AGCCTGAGCAGCAATGGCAATGTGACA |
| | | | | GAGTCTGGCTGTAAGGAGTGTGAGGAG |
| | | | | CTGGAGGAGAAGAACATCAAGGAGTTT |
| | | | | CTGCAGAGCTTTGTGCACATCGTGCAG |
| | | | | ATGTTCATCAATACAAGC |
| IL-15Rα | 105 | ITCPPPMSVEHADI | 108 | ATTACATGCCCTCCTCCAATGTCTGTG |
| | | WVKSYSLYSRERYI | | GAGCACGCCGATATTTGGGTGAAGTCC |
| | | CNSGFKRKAGTSSL | | TACAGCCTGTACAGCAGAGAGAGATAC |
| | | TECVLNKATNVAHW | | ATCTGCAACAGCGGCTTTAAGAGAAAG |
| | | TTPSLKCIRDPALV | | GCCGGCACCTCTTCTCTGACAGAGTGC |
| | | HQRPAPPSTVTTAG | | GTGCTGAATAAGGCCACAAATGTGGCC |
| | | VTPQPESLSPSGKE | | CACTGGACAACACCTAGCCTGAAGTGC |
| | | PAASSPSSNNTAAT | | ATTAGAGATCCTGCCCTGGTCCACCAG |
| | | TAAIVPGSQLMPSK | | AGGCCTGCCCCTCCATCTACAGTGACA |
| | | SPSTGTTEISSHES | | ACAGCCGGAGTGACACCTCAGCCTGAA |
| | | SHGTPSQTTAKNWE | | TCTCTGAGCCCTTCTGGAAAAGAACCT |
| | | LTASASHQPPGVYP | | GCCGCCAGCTCTCCTAGCTCTAATAAT |
| | | QGHSDTTVAISTST | | ACCGCCGCCACAACAGCCGCCATTGTG |
| | | VLLCGLSAVSLLAC | | CCTGGATCTCAGCTGATGCCTAGCAAG |
| | | YLKSRQTPPLASVE | | TCTCCTAGCACAGGCACAACAGAGATC |
| | | MEAMEALPVTWGTS | | AGCAGCCACGAATCTTCTCACGGAACA |
| | | SRDEDLENCSHHL | | CCTTCTCAGACCACCGCCAAGAATTGG |
| | | | | GAGCTGACAGCCTCTGCCTCTCACCAG |
| | | | | CCTCCAGGAGTGTATCCTCAGGGCCAC |
| | | | | TCTGATACAACAGTGGCCATCAGCACA |
| | | | | TCTACAGTGCTGCTGTGTGGACTGTCT |
| | | | | GCCGTGTCTCTGCTGGCCTGTTACCTG |
| | | | | AAGTCTAGACAGACACCTCCTCTGGCC |
| | | | | TCTGTGGAGATGGAGGCCATGGAAGCC |
| | | | | CTGCCTGTGACATGGGGAACAAGCAGC |
| | | | | AGAGATGAGGACCTGGAGAATTGTTCT |
| | | | | CACCACCTG |
| HUMAN | 146 | ASTKGPSVFPLAPC | | |
| Immuno- | | SRSTSESTAALGCL | | |
| globulin | | VKDYFPEPVTVSWN | | |
| heavy | | SGALTSGVHTFPAV | | |
| constant | | LQSSGLYSLSSVVT | | |
| gamma 4 | | VPSSSLGTKTYTCN | | |
| (IgG4 | | VDHKPSNTKVDKRV | | |
| CH) | | ESKYGPPCPSCPAP | | |
| | | EFLGGPSVFLFPPK | | |
| | | PKDTLMISRTPEVT | | |
| | | CVVVDVSQESPEVQ | | |
| | | FNWYVDGVEVHNAK | | |
| | | TKPREEQFNSTYRV | | |
| | | VSVLTVLHQDWLNG | | |
| | | KEYKCKVSNKGLPS | | |
| | | SIEKTISKAKGQPR | | |
| | | EPQVYTLPPSQEEM | | |
| | | TK | | |
| | | NQVSLTCLVKGFYP | | |
| | | SDIAVEWESNGQPE | | |
| | | NNYKTTPPVLDSDG | | |
| | | SFFLYSRLTVDKSR | | |
| | | WQEGNVFSCSVMHE | | |
| | | ALHNHYTQKSLSLS | | |
| | | LGK | | |
| IgG4 | 147 | ASTKGPSVFPLAPC | 117 | GCTAGCACCAAGGGCCCATCGGTCTTC |
| CH | | SRSTSESTAALGCL | | CCCCTGGCGCCCTGCTCCAGGAGCACC |
| (S108P, | | VKDYFPEPVTVSWN | | TCCGAGAGCACAGCCGCCCTGGGCTGC |
| L325P) | | SGALTSGVHTFPAV | | CTGGTCAAGGACTACTTCCCCGAACCG |
| | | LQSSGLYSLSSVVT | | GTGACGGTGTCGTGGAACTCAGGCGCC |
| | | VPSSSLGTKTYTCN | | CTGACCAGCGGCGTGCACACCTTCCCG |
| | | VDHKPSNTKVDKRV | | GCTGTCCTACAGTCCTCAGGACTCTAC |
| | | ESKYGPPCPPCPAP | | TCCCTCAGCAGCGTGGTGACCGTGCCC |
| | | EFLGGPSVFLFPPK | | TCCAGCAGCTTGGGCACGAAGACCTAC |
| | | PKDTLMISRTPEVT | | ACCTGCAACGTAGATCACAAGCCCAGC |
| | | CVVVDVSQEDPEVQ | | AACACCAAGGTGGACAAGAGAGTTGAG |
| | | FNWYVDGVEVHNAK | | TCCAAATATGGTCCCCCATGCCCACCA |
| | | TKPREEQFNSTYRV | | TGCCCAGCACCTGAGTTCCTGGGGGGA |
| | | VSVLTVLHQDWLNG | | CCATCAGTCTTCCTGTTCCCCCCAAAA |
| | | KEYKCKVSNKGLPS | | CCCAAGGACACTCTCATGATCTCCCGG |
| | | SIEKTISKAKGQPR | | ACCCCTGAGGTCACGTGCGTGGTGGTG |
| | | EPQVYTLPPSQEEM | | GACGTGAGCCAGGAAGACCCCGAGGTC |
| | | TKNQVSLTCLVKGF | | CAGTTCAACTGGTACGTGGATGGCGTG |
| | | YPSDIAVEWESNGQ | | GAGGTGCATAATGCCAAGACAAAGCCG |
| | | PENNYKTTPPVLDS | | CGGGAGGAGCAGTTCAACAGCACGTAC |
| | | DGSFFLYSRLTVDK | | CGTGTGGTCAGCGTCCTCACCGTCCTG |
| | | SRWQEGNVFSCSVM | | CACCAGGACTGGCTGAACGGCAAGGAG |
| | | HEALHNHYTQKSLS | | TACAAGTGCAAGGTCTCCAACAAAGGC |
| | | LSPGK | | CTCCCGTCCTCCATCGAGAAAACCATC |
| | | | | TCCAAAGCCAAAGGGCAGCCCCGAGAG |
| | | | | CCACAGGTGTACACCCTGCCCCCATCC |
| | | | | CAGGAGGAGATGACCAAGAACCAGGTC |
| | | | | AGCCTGACCTGCCTGGTCAAAGGCTTC |
| | | | | TACCCCAGCGACATCGCCGTGGAGTGG |
| | | | | GAGAGCAATGGGCAGCCGGAGAACAAC |
| | | | | TACAAGACCACGCCTCCCGTGCTGGAC |
| | | | | TCCGACGGCTCCTTCTTCCTCTACAGC |
| | | | | AGGCTCACCGTGGACAAGAGCAGGTGG |
| | | | | CAGGAGGGGAATGTCTTCTCATGCTCC |
| | | | | GTGATGCATGAGGCTCTGCACAACCAC |
| | | | | TACACACAGAAGAGCCTCTCCCTGTCT |
| | | | | CCGGGTAAA |
| IgG4 | 291 | ASTKGPSVFPLAPC | | |
| CH | | SRSTSESTAALGCL | | |
| (S108P) | | VKDYFPEPVTVSWN | | |
| | | SGALTSGVHTFPAV | | |
| | | LQSSGLYSLSSVVT | | |
| | | VPSSSLGTKTYTCN | | |
| | | VDHKPSNTKVDKRV | | |
| | | ESKYGPPCPPCPAP | | |
| | | EFLGGPSVFLFPPK | | |
| | | PKDTLMISRTPEVT | | |
| | | CVVVDVSQEDPEVQ | | |
| | | FNWYVDGVEVHNAK | | |
| | | TKPREEQFNSTYRV | | |
| | | VSVLTVLHQDWLNG | | |
| | | KEYKCKVSNKGLPS | | |
| | | SIEKTISKAKGQPR | | |
| | | EPQVYTLPPSQEEM | | |
| | | TKNQVSLTCLVKGF | | |
| | | YPSDIAVEWESNGQ | | |
| | | PENNYKTTPPVLDS | | |
| | | DGSFFLYSRLTVDK | | |
| | | SRWQEGNVFSCSVM | | |
| | | HEALHNHYTQKSLS | | |
| | | LSLGK | | |
| IgG4 | 292 | ASTKGPSVFPLAPC | | |
| CH | | SRSTSESTAALGCL | | |
| (L325P) | | VKDYFPEPVTVSWN | | |
| | | SGALTSGVHTFPAV | | |
| | | LQSSGLYSLSSVVT | | |
| | | VPSSSLGTKTYTCN | | |
| | | VDHKPSNTKVDKRV | | |
| | | ESKYGPPCPSCPAP | | |
| | | EFLGGPSVFLFPPK | | |
| | | PKDTLMISRTPEVT | | |
| | | CVVVDVSQEDPEVQ | | |
| | | FNWYVDGVEVHNAK | | |
| | | TKPREEQFNSTYRV | | |
| | | VSVLTVLHQDWLNG | | |
| | | KEYKCKVSNKGLPS | | |
| | | SIEKTISKAKGQPR | | |
| | | EPQVYTLPPSQEEM | | |
| | | TKNQVSLTCLVKGF | | |
| | | YPSDIAVEWESNGQ | | |
| | | PENNYKTTPPVLDS | | |
| | | DGSFFLYSRLTVDK | | |
| | | SRWQEGNVFSCSVM | | |
| | | HEALHNHYTQKSLS | | |
| | | LSPGK | | |
| Anti- | | | 118 | GAGATAGTTATGACTCAAAGCCCCGCT |
| PD-1 | | | | ACATTATCCCTGTCTCCGGGTGAACGG |
| (nVL1) | | | | GCCACCCTGTCATGCCGGGCTTCACAG |
| | | | | TCAGTGTCAAGCTATCTGGCATGGTAT |
| | | | | CAGCAGAAGCCTGGACAGGCCCCAAGG |
| | | | | CTACTGATTTATGACGCCAGCAACCGC |
| | | | | GCTACAGGTATTCCTGCTAGGTTCTCA |
| | | | | GGGTCAGGCTCTGGAACCGACTTTACT |
| | | | | CTGACTATCTCCTCTCTTGAACCCGAG |
| | | | | GATTTCGCGGTGTACTACTGTCAGCAG |
| | | | | TATAATAACTGGCCACGCACATTCGGC |
| | | | | GAGGGCACTAAAGTCGAAATTAAG |
| Anti- | | | 119 | GAGATCGTACTGACTCAGTCTCCAGCC |
| PD-1 | | | | ACATTGTCCCTGTCCCCAGGGGAGCGC |
| (nVL2) | | | | GCCACCCTGAGCTGTAGAGCTTCACAG |
| | | | | TCCGTCAGTTCTTACCTCGCGTGGTAT |
| | | | | CAGCAAAAACCTGGACAAGCTCCGAGG |
| | | | | TTGCTTATCTATGACGCCTCCAACCGC |
| | | | | GCCACTGGCATACCAGCAAGGTTCAGC |
| | | | | GGATCTGGGTCCGGCACAGATTTTACC |
| | | | | CTCACTATTTCTAGCCTTGAGCCGGAA |
| | | | | GATTTCGCTGTTTACTACTGCCAGCAG |
| | | | | CGATCCAACTGGCCCAAGACATTCGGC |
| | | | | CAGGGAACTAAAGTGGAAATCAAA |
| Anti- | 148 | EIVLTQSPATLSLS | 120 | GAAATTGTGTTGACACAGTCTCCAGCC |
| PD-1 | | PGERATLSCRASQS | | ACCCTGTCTTTGTCTCCAGGGGAAAGA |
| (nVL3) | | VSSYLAWYQQKPGQ | | GCCACCCTCTCCTGCAGGGCCAGTCAG |
| | | APRLLIYDASNRAN | | AGTGTTAGTAGTTACTTAGCCTGGTAC |
| | | GIPARFSGSGSGTD | | CAACAGAAACCTGGCCAGGCTCCCAGG |
| | | FTLTISSLEPEDFA | | CTCCTCATCTATGATGCATCCAACAGG |
| | | VYYCQQSSNWPRTF | | GCCAACGGCATCCCAGCCAGGTTCAGT |
| | | GQGTKVEIKR | | GGCAGTGGGTCTGGGACAGACTTCACT |
| | | | | CTCACCATCAGCAGCCTAGAGCCTGAA |
| | | | | GATTTCGCAGTTTATTACTGTCAGCAG |
| | | | | AGTAGCAACTGGCCTCGGACGTTCGGC |
| | | | | CAAGGGACCAAGGTGGAAATCAAAAGA |
| Anti- | | | 121 | CGGAACGTGCTGACCCAGTCCCCCCTT |
| PD-1 | | | | AGCCTCCCCGTCACGCCCGGAGAGCCC |
| (pVL1) | | | | GCAAGTATCAGCTGCCGCAGTTCACAA |
| | | | | AGTCTGAGTTCTTCTGGATACACCTAT |
| | | | | TTGGACTGGTATTTGCAGAAGCCAGGG |
| | | | | CAATCCCCACAGCTCCTGATATACCTC |
| | | | | GCAAGCTGGAGAGATAGCGGAGTACCT |
| | | | | GATCGCTTTTCTGGTAGCGGATCTGGT |
| | | | | ACGGATTTCACTCTGAAGATTTCTAGG |
| | | | | GTGGAGGCGGAGGACGTGGGAGTGTAC |
| | | | | TACTGTATGCAAGCCGAGCAGACTCCC |
| | | | | GGCCCAGGTAACACGTTCGGACAGGGG |
| | | | | ACCAAACTGGAGATTAAG |
| Anti- | | | 122 | GATGTGGTAATGACCCAGTCACCTCTT |
| PD-1 | | | | TCACTGCCTGTCACTCCCGGAGAGCCA |
| (pVL2) | | | | GCTTCAATCTCCTGCCGTAGCTCTCAA |
| | | | | TCATTGTTGCACACCAATGGATACAAC |
| | | | | TACCTCCACTGGTATCTCCAGAAGCCC |
| | | | | GGACAAAGCCCGCAGCTGCTGATCTAC |
| | | | | CTGGGCAGCTGGCAGGACTCCGGGGTG |
| | | | | CCCGACCGATTTAGCGGCAGTGGGAGC |
| | | | | GGCACGGACTTTACACTGAAGATCAGC |
| | | | | CGAGTAGAGGCGGAGGACGTGGGCGTT |
| | | | | TACTACTGTATGCAGGCAGAGCAGACC |
| | | | | CCCAGAACCTTCGGCCAGGGCACCCGG |
| | | | | CTGGAGGTGAAA |
| Anti- | | | 123 | CAGGTGCAGTTGGTTGAAAGCGGAGGA |
| PD-1 | | | | GGCGTGGTTCAACCCGGTAGAAGCCTA |
| (nVH1) | | | | CGGCTGTCATGTGCGGCCTCCGGCTTC |
| | | | | ACATTTCGATCTTACGGAATGCACTGG |
| | | | | GTCAGGCAGGCACCCGGCAAGGGTCTG |
| | | | | GAGTGGGTCGCCATAATTTTCTATGAC |
| | | | | GGCAGCAACAAGTATTACGCCGACAGT |
| | | | | GTTAAGGGGCGGTTTACCATCAGCAGA |
| | | | | GACAACTCTAAAAACACTCTTTATCTG |
| | | | | CAAATGAACTCTCTGCGGGCAGAGGAT |
| | | | | ACCGCTGTTTACTATTGCGCCAGAGAT |
| | | | | GACGACTACTGGGGGCAGGGTGCCTTG |
| | | | | GTGACTGTGAGCAGC |
| Anti- | | | 124 | GAGGTCCAGTTAGTCCAAAGCGGCGGA |
| PD-1 | | | | GGCGTAGTGCAACCTGGCAGAAGCCTG |
| (nVH2) | | | | CGGTTATCGTGCGCCGCAAGCGGCTTC |
| | | | | ACCTTTAGCTCTTATGGTATGCACTGG |
| | | | | GTCAGACAGGCCCCTGGGAAGGGCCTG |
| | | | | GAGTGGGTGGCCGTGATCTGGTATGAC |
| | | | | GGGAGCAACAAGTATTACGCGGATTCC |
| | | | | GTCAAGGGACGGTTCACCATATCCCGC |
| | | | | GATAACAGCAAGAATACTCTTTACTTA |
| | | | | CAGATGAACAGCCTGAGGGCCGAGGAC |
| | | | | ACCGCAGTATATTATTGCGCTGGCGAA |
| | | | | GGCTTTGACTATTGGGGTCAGGGCACT |
| | | | | CTGGTGACTGTGAGCAGC |
| Anti- | 149 | QVQLVESGGGVVQP | 125 | CAGGTGCAGCTGGTGGAGTCTGGGGGA |
| PD-1 | | GRSLRLDCKASGIT | | GGCGTGGTCCAGCCTGGGAGGTCCCTG |
| (nVH3) | | FSNSGMHWVRQAPG | | AGACTCGACTGTAAAGCGTCTGGAATC |
| | | KGLEWVAVIWYDGS | | ACCTTCAGTAACTCTGGCATGCACTGG |
| | | KRYYADSVKGRFTI | | GTCCGCCAGGCTCCAGGCAAGGGGCTG |
| | | SRDNSKNTLFLQMN | | GAGTGGGTGGCAGTTATTTGGTATGAT |
| | | SLRAEDTAVYYCAT | | GGAAGTAAAAGATACTATGCAGACTCC |
| | | NNDYWGQGTLVTVS | | GTGAAGGGCCGATTCACCATCTCCAGA |
| | | S | | GACAATTCCAAGAACACGCTGTTTCTG |
| | | | | CAAATGAACAGCCTGAGAGCCGAGGAC |
| | | | | ACGGCTGTGTATTACTGTGCGACAAAC |
| | | | | AACGACTACTGGGGCCAGGGAACCCTG |
| | | | | GTCACCGTCTCCTCA |
| Anti- | 150 | QVQLVESGGGVVQP | 126 | CAGGTGCAGCTGGTGGAGTCTGGGGGA |
| PD-1 | | GRSLRLDCKASGIT | | GGCGTGGTCCAGCCTGGGAGGTCCCTG |
| (nVH4) | | FSNSGMHWVRQAPG | | AGACTCGACTGTAAAGCGTCTGGAATC |
| | | KGLEWVAVIWYDGS | | ACCTTCAGTAACTCTGGCATGCACTGG |
| | | KRYYADSVKGRFTI | | GTCCGCCAGGCTCCAGGCAAGGGGCTG |
| | | SRDNSKNTLFLQMN | | GAGTGGGTGGCAGTTATTTGGTATGAT |
| | | SLRAEDTAVYYCAR | | GGAAGTAAAAGATACTATGCAGACTCC |
| | | NDDYWGQGTLVTVS | | GTGAAGGGCCGATTCACCATCTCCAGA |
| | | S | | GACAATTCCAAGAACACGCTGTTTCTG |
| | | | | CAAATGAACAGCCTGAGAGCCGAGGAC |
| | | | | ACGGCTGTGTATTACTGTGCGAGAAAC |
| | | | | GACGACTACTGGGGCCAGGGAACCCTG |
| | | | | GTCACCGTCTCCTCA |
| Anti- | 151 | QVQLVQSGAEVKKP | 127 | CAGGTACAGCTGGTGCAGAGCGGCGCA |
| PD-1 | | GASVKVSCKASGYT | | GAGGTGAAGAAGCCAGGCGCTTCTGTA |
| (pVH1) | | FTGYYMHWVRQAPG | | AAGGTATCCTGCAAGGCATCCGGGTAT |
| | | QGLEWMGAINPNSG | | ACTTTCACCGGCTATTACATGCACTGG |
| | | GTNYAQKFQGRVTM | | GTTCGTCAGGCACCCGGCCAGGGACTA |
| | | TRDTSISTAYMELS | | GAATGGATGGGGGCCATCAACCCTAAT |
| | | RLRSDDTAVYYCAR | | AGTGGCGGTACTAACTACGCACAAAAG |
| | | RPDRANWHFDYWGQ | | TTTCAGGGGCGAGTGACCATGACTCGG |
| | | GTLVTVSS | | GATACCTCCATCTCCACGGCATACATG |
| | | | | GAGCTGAGTCGCTTGCGGTCAGATGAC |
| | | | | ACTGCGGTGTACTACTGCGCTCGCAGG |
| | | | | CCCGACCGAGCTAATTGGCACTTTGAC |
| | | | | TACTGGGGACAGGGTACACTGGTGACC |
| | | | | GTGTCATCA |
| Anti- | 152 | QVQLVQSGAEVKKP | 128 | CAGGTGCAGCTGGTCCAGAGCGGCGCG |
| PD-1 | | GASVKVSCKASGYT | | GAAGTGAAAAAGCCCGGCGCTTCCGTG |
| (pVH2) | | FTGYYMHWVRQAPG | | AAGGTTTCTTGCAAAGCCTCTGGATAC |
| | | QGLEWMGAINPNSG | | ACATTCACTGGCTATTATATGCACTGG |
| | | GTNYAQKLQGRVTM | | GTCAGACAGGCCCCCGGCCAGGGATTG |
| | | TTDTSTSTAYMELR | | GAGTGGATGGGTGCAATCAACCCCAAT |
| | | SLRSDDTAVYYCAR | | TCTGGTGGGACCAATTACGCACAGAAA |
| | | HGLKGDGYFDYWGQ | | CTCCAGGGCCGAGTGACAATGACCACC |
| | | GTLVTVSS | | GACACTTCTACCAGCACTGCCTACATG |
| | | | | GAGCTGCGGTCTCTGCGATCAGACGAC |
| | | | | ACCGCTGTGTACTATTGTGCAAGACAC |
| | | | | GGGCTGAAGGGCGACGGCTATTTTGAC |
| | | | | TACTGGGGACAGGGCACGCTGGTTACC |
| | | | | GTGAGTTCC |
| Anti- | 153 | QVQLVQSGVEVKKP | 129 | CAGGTCCAGCTCGTGCAAAGCGGAGTG |
| PD-1 | | GASVKVSCKASGYT | | GAAGTGAAAAAGCCTGGCGCTTCCGTC |
| (pVH3) | | FTNYYMYWVRQAPG | | AAGGTCAGCTGTAAGGCCAGCGGATAC |
| | | QGLEWMGGINPSNG | | ACATTCACAAACTATTACATGTACTGG |
| | | GTNFNEKFKNRVTL | | GTGAGGCAGGCTCCCGGACAGGGACTG |
| | | TTDSSTTTAYMELK | | GAATGGATGGGCGGAATCAATCCCTCC |
| | | SLQFDDTAVYYCAR | | AACGGAGGCACAAACTTTAACGAAAAG |
| | | RDYRFYMGFDYWGQ | | TTTAAGAATAGAGTCACCCTCACCACA |
| | | GTTVTVSS | | GACTCCAGCACAACCACAGCCTATATG |
| | | | | GAACTGAAAAGCCTCCAGTTTGACGAT |
| | | | | ACCGCTGTGTATTACTGTGCCAGGAGA |
| | | | | GATTACAGGTTCTACATGGGATTCGAT |
| | | | | TACTGGGGCCAAGGCACAACCGTCACC |
| | | | | GTCAGCTCC |
| Anti- | 154 | QVQLVQSGVEVKKP | 130 | CAGGTCCAGCTCGTGCAAAGCGGAGTG |
| PD-1 | | GASVKVSCKASGYT | | GAAGTGAAAAAGCCTGGCGCTTCCGTC |
| (pVH4) | | FTNYYMYWVRQAPG | | AAGGTCAGCTGTAAGGCCAGCGGATAC |
| | | QGLEWMGGINPSNG | | ACATTCACAAACTATTACATGTACTGG |
| | | GTNFNEKFKNRVTL | | GTGAGGCAGGCTCCCGGACAGGGACTG |
| | | TTDSSTTTAYMELK | | GAATGGATGGGCGGAATCAATCCCTCC |
| | | SLQFDDTAVYYCAR | | AACGGAGGCACAAACTTTAACGAAAAG |
| | | RDYRFNMGFDYWGQ | | TTTAAGAATAGAGTCACCCTCACCACA |
| | | GTTVTVSS | | GACTCCAGCACAACCACAGCCTATATG |
| | | | | GAACTGAAAAGCCTCCAGTTTGACGAT |
| | | | | ACCGCTGTGTATTACTGTGCCAGGAGA |
| | | | | GATTACAGGTTCAACATGGGATTCGAT |
| | | | | TACTGGGGCCAAGGCACAACCGTCACC |
| | | | | GTCAGCTCC |
| Anti- | 155 | QVQLVQSGVEVKKP | 131 | CAGGTCCAGCTCGTGCAAAGCGGAGTG |
| PD-1 | | GASVKVSCKASGYT | | GAAGTGAAAAAGCCTGGCGCTTCCGTC |
| (pVH5) | | FTNYYMYWVRQAPG | | AAGGTCAGCTGTAAGGCCAGCGGATAC |
| | | QGLEWMGGIQPSNG | | ACATTCACAAACTATTACATGTACTGG |
| | | GTNFNEKFKNRVTL | | GTGAGGCAGGCTCCCGGACAGGGACTG |
| | | TTDSSTTTAYMELK | | GAATGGATGGGCGGAATCCAGCCCTCC |
| | | SLQFDDTAVYYCAR | | AACGGAGGCACAAACTTTAACGAAAAG |
| | | RDYRFYMGFDYWGQ | | TTTAAGAATAGAGTCACCCTCACCACA |
| | | GTTVTVSS | | GACTCCAGCACAACCACAGCCTATATG |
| | | | | GAACTGAAAAGCCTCCAGTTTGACGAT |
| | | | | ACCGCTGTGTATTACTGTGCCAGGAGA |
| | | | | GATTACAGGTTCTACATGGGATTCGAT |
| | | | | TACTGGGGCCAAGGCACAACCGTCACC |
| | | | | GTCAGCTCC |
| Anti- | 156 | QVQLVQSGVEVKKP | 132 | CAGGTCCAGCTCGTGCAAAGCGGAGTG |
| PD-1 | | GASVKVSCKASGYT | | GAAGTGAAAAAGCCTGGCGCTTCCGTC |
| (pVH6) | | FTNYYMYWVRQAPG | | AAGGTCAGCTGTAAGGCCAGCGGATAC |
| | | QGLEWMGGIDPSNG | | ACATTCACAAACTATTACATGTACTGG |
| | | GTNFNEKFKNRVTL | | GTGAGGCAGGCTCCCGGACAGGGACTG |
| | | TTDSSTTTAYMELK | | GAATGGATGGGCGGAATCGACCCCTCC |
| | | SLQFDDTAVYYCAR | | AACGGAGGCACAAACTTTAACGAAAAG |
| | | YDYRFDMGFDYWGQ | | TTTAAGAATAGAGTCACCCTCACCACA |
| | | GTTVTVSS | | GACTCCAGCACAACCACAGCCTATATG |
| | | | | GAACTGAAAAGCCTCCAGTTTGACGAT |
| | | | | ACCGCTGTGTATTACTGTGCCAGGTAC |
| | | | | GATTACAGGTTCGATATGGGATTCGAT |
| | | | | TACTGGGGCCAAGGCACAACCGTCACC |
| | | | | GTCAGCTCC |
| Anti- | 157 | QVQLVESGGGVVQP | 133 | CAGGTGCAGCTGGTGGAGTCTGGGGGA |
| PD-1 | | GRSLRLDCKASGFT | | GGCGTGGTCCAGCCTGGGAGGTCCCTG |
| (nVH7) | | FSNSGMHWVRQAPG | | AGACTCGACTGTAAAGCGTCTGGATTC |
| | | KGLEWVAVIWYDGS | | ACCTTCAGTAACTCTGGCATGCACTGG |
| | | KRYYADSVKGRFTI | | GTCCGCCAGGCTCCAGGCAAGGGGCTG |
| | | SRDNSKNTLFLQMN | | GAGTGGGTGGCAGTTATTTGGTATGAT |
| | | SLRAEDTAVYYCAT | | GGAAGTAAAAGATACTATGCAGACTCC |
| | | NNDYWGQGTLVTVS | | GTGAAGGGCCGATTCACCATCTCCAGA |
| | | S | | GACAATTCCAAGAACACGCTGTTTCTG |
| | | | | CAAATGAACAGCCTGAGAGCCGAGGAC |
| | | | | ACGGCTGTGTATTACTGTGCGACAAAC |
| | | | | AACGACTACTGGGGCCAGGGAACCCTG |
| | | | | GTCACCGTCTCCTCA |
| Anti- | 158 | EVQLVQSGGGVVQP | 134 | GAGGTCCAGTTAGTCCAAAGCGGCGGA |
| PD-1 | | GRSLRLSCAASGFT | | GGCGTAGTGCAACCTGGCAGAAGCCTG |
| (nVH8) | | FSSYGMHWVRQAPG | | CGGTTATCGTGCGCCGCAAGCGGCTTC |
| | | KGLEWVAVIWYDGS | | ACCTTTAGCTCTTATGGTATGCACTGG |
| | | NKYYADSVKGRFTI | | GTCAGACAGGCCCCTGGGAAGGGCCTG |
| | | SRDNSKNTLYLQMN | | GAGTGGGTGGCCGTGATCTGGTATGAC |
| | | SLRAEDTAVYYCAT | | GGGAGCAACAAGTATTACGCGGATTCC |
| | | NNDYWGQGTLVTVS | | GTCAAGGGACGGTTCACCATATCCCGC |
| | | S | | GATAACAGCAAGAATACTCTTTACTTA |
| | | | | CAGATGAACAGCCTGAGGGCCGAGGAC |
| | | | | ACCGCAGTATATTATTGCGCTACCAAT |
| | | | | AATGACTATTGGGGTCAGGGCACTCTG |
| | | | | GTGACTGTGAGCAGC |
| Anti- | 159 | EVQLVQSGGGVVQP | 135 | GAGGTGCAACTGGTTCAGTCCGGCGGG |
| PD-1 | | GRSLRLSCAASGIT | | GGCGTCGTCCAGCCGGGGCGCAGTCTG |
| (nVH9) | | FSNSMHWVRQAPGK | | CGCTTGAGCTGTGCTGCCTCTGGGATT |
| | | GLEWVAVIWYDGSK | | ACCTTTAGCAACTCCATGCATTGGGTG |
| | | RYYADSVKGRFTIS | | CGGCAGGCACCCGGGAAGGGACTGGAA |
| | | RDNSKNTLYLQMNS | | TGGGTCGCAGTGATCTGGTACGATGGA |
| | | LRAEDTAVYYCATN | | TCAAAGCGGTATTACGCCGACTCCGTC |
| | | NDYWGQGTLVTVSS | | AAAGGCCGGTTCACAATCAGCCGCGAC |
| | | | | AACAGCAAAAATACTTTATATCTTCAG |
| | | | | ATGAATTCCCTTAGGGCAGAGGATACT |
| | | | | GCTGTGTATTACTGCGCTACTAACAAC |
| | | | | GATTATTGGGGGCAGGGGACACTAGTC |
| | | | | ACTGTTTCTAGT |
| Anti- | 160 | EVQLVQSGGGVVQP | 136 | GAGGTCCAGTTAGTCCAAAGCGGCGGA |
| PD-1 | | GRSLRLSCAASGFT | | GGCGTAGTGCAACCTGGCAGAAGCCTG |
| (nVH10) | | FSNSGMHWVRQAPG | | CGGTTATCGTGCGCCGCAAGCGGCTTC |
| | | KGLEWVAVIWYDGS | | ACCTTTAGCAATTCTGGTATGCACTGG |
| | | NKYYADSVKGRFTI | | GTCAGACAGGCCCCTGGGAAGGGCCTG |
| | | SRDNSKNTLYLQMN | | GAGTGGGTGGCCGTGATCTGGTATGAC |
| | | SLRAEDTAVYYCAT | | GGGAGCAACAAGTATTACGCGGATTCC |
| | | NNDYWGQGTLVTVS | | GTCAAGGGACGGTTCACCATATCCCGC |
| | | S | | GATAACAGCAAGAATACTCTTTACTTA |
| | | | | CAGATGAACAGCCTGAGGGCCGAGGAC |
| | | | | ACCGCAGTATATTATTGCGCTACCAAT |
| | | | | AATGACTATTGGGGTCAGGGCACTCTG |
| | | | | GTGACTGTGAGCAGC |
| Anti- | 161 | EVQLVQSGGGVVQP | 137 | GAGGTCCAGTTAGTCCAAAGCGGCGGA |
| PD-1 | | GRSLRLSCAASGFT | | GGCGTAGTGCAACCTGGCAGAAGCCTG |
| (nVH11) | | FSSYGMHWVRQAPG | | CGGTTATCGTGCGCCGCAAGCGGCTTC |
| | | KGLEWVAVIWYDGS | | ACCTTTAGCTCTTATGGTATGCACTGG |
| | | KYYADSVKGRFTIS | | GTCAGACAGGCCCCTGGGAAGGGCCTG |
| | | RDNSKNTLYLQMNS | | GAGTGGGTGGCCGTGATCTGGTATGAC |
| | | LRAEDTAVYYCATN | | GGGAGCAAGTATTACGCGGATTCCGTC |
| | | NDYWGQGTLVTVSS | | AAGGGACGGTTCACCATATCCCGCGAT |
| | | | | AACAGCAAGAATACTCTTTACTTACAG |
| | | | | ATGAACAGCCTGAGGGCCGAGGACACC |
| | | | | GCAGTATATTATTGCGCTACCAATAAT |
| | | | | GACTATTGGGGTCAGGGCACTCTGGTG |
| | | | | ACTGTGAGCAGC |
| Anti- | 162 | QVQLVESGGGVVQP | 138 | CAGGTGCAGCTGGTGGAGTCTGGGGGA |
| PD-1 | | GRSLRLDCKASGIT | | GGCGTGGTCCAGCCTGGGAGGTCCCTG |
| (nVH12) | | FSNSGMHWVRQAPG | | AGACTCGACTGTAAAGCGTCTGGAATC |
| | | KGLEWVAVIWYDGS | | ACCTTCAGTAACTCTGGCATGCACTGG |
| | | NKRYYADSVKGRFT | | GTCCGCCAGGCTCCAGGCAAGGGGCTG |
| | | ISRDNSKNTLFLQM | | GAGTGGGTGGCAGTTATTTGGTATGAT |
| | | NSLRAEDTAVYYCA | | GGAAGTAACAAAAGATACTATGCAGAC |
| | | TNNDYWGQGTLVTV | | TCCGTGAAGGGCCGATTCACCATCTCC |
| | | SS | | AGAGACAATTCCAAGAACACGCTGTTT |
| | | | | CTGCAAATGAACAGCCTGAGAGCCGAG |
| | | | | GACACGGCTGTGTATTACTGTGCGACA |
| | | | | AACAACGACTACTGGGGCCAGGGAACC |
| | | | | CTGGTCACCGTCTCCTCA |
| Anti- | 163 | QVQLVESGGGVVQP | 139 | CAGGTGCAGCTGGTGGAGTCTGGGGGA |
| PD-1 | | GRSLRLDCKASGFT | | GGCGTGGTCCAGCCTGGGAGGTCCCTG |
| (nVH12) | | FSNSGMHWVRQAPG | | AGACTCGACTGTAAAGCGTCTGGATTC |
| | | KGL | | ACCTTCAGTAACTCTGGCATGCACTGG |
| | | | | GTCCGCCAGGCTCCAGGCAAGGGGCTG |
| | | | | GAGTGGGTGGCAGTTATTTGGTATGAT |
| | | | | GGAAGTAACAAAAGATACTATGCAGAC |
| | | | | TCCGTGAAGGGCCGATTCACCATCTCC |
| | | | | AGAGACAATTCCAAGAACACGCTGTTT |
| | | | | CTGCAAATGAACAGCCTGAGAGCCGAG |
| | | | | GACACGGCTGTGTATTACTGTGCGACA |
| | | | | AACAACGACTACTGGGGCCAGGGAACC |
| | | | | CTGGTCACCGTCTCCTCA |
| Anti- | 164 | EVQLVQSGGGVVQP | 140 | GAGGTGCAACTTGTGCAAAGCGGCGGC |
| PD-1 | | GRSLRLSCAASGIT | | GGAGTCGTGCAGCCCGGTCGATCTCTT |
| (nVH14) | | FSNSGMHWVRQAPG | | CGCCTGAGTTGTGCTGCCAGCGGCATT |
| | | KGLEWVAVIWYDGS | | ACCTTTAGCAATTCTGGTATGCACTGG |
| | | KYYADSVKGRFTIS | | GTACGTCAGGCCCCCGGTAAGGGGCTA |
| | | RDNSKNTLYLQMNS | | GAATGGGTGGCTGTGATTTGGTACGAT |
| | | LRAEDTAVYYCATN | | GGTTCTAAGTACTACGCCGACAGCGTT |
| | | NDYWGQGTLVTVSS | | AAAGGCCGATTCACCATCAGTAGAGAC |
| | | | | AACAGTAAGAACACCCTCTACCTCCAG |
| | | | | ATGAACAGTCTGCGAGCTGAAGACACT |
| | | | | GCTGTGTACTACTGTGCCACCAACAAC |
| | | | | GACTACTGGGGACAGGGAACCCTGGTC |
| | | | | ACCGTGAGTAGT |

Table 14. Exemplary Anti-TGFB V_{H} and V_{L} Sequences

Table 14 provides exemplary TGFB receptor sequences for use in anti-PD-1 fusion proteins described herein. In any embodiments exemplifying a fusion protein with. e.g., a TGFβRII ECD, an anti-TGFB antibody (e.g., scFv, Fab) may be employed instead.

| | SEQ | | SEQ | |
|---|---|---|---|---|
| Name | ID NO | Amino Acid Sequence | ID NQ | Nucleotide Sequence |
| Anti- | 165 | DIMMTQSPSSLAVS | 179 | GATATAATGACACAGAGCCCCA |
| TGFb | | AGEKVTMSCKSSQS | | GCTCTCTAGCTGTGAGTGCTGG |
| clone 1 | | VLYSSNQKNYLAW | | CGAGAAGGTGACCATGAGCTGT |
| VL | | YQQKPGQSPKLLIY | | AAGAGCAGTCAAAGCGTGCTGT |
| | | WASTRESGVPDRFT | | ACAGTTCCAATCAGAAAAATTA |
| | | GSGSGTDFTLTISSV | | CCTCGCATGGTATCAGAAGCCA |
| | | QAEDLAVYYCHQY | | GGTCAAAGCCCTAAGCTCCTTA |
| | | LSSDTFGGGTKLEIK | | TCTACTGGGCCTCAACCCGTGA |
| | | RTVA | | AAGTGGAGTGCCTGACAGATTT |
| | | | | ACTGGTTCAGGGAGCGGCACCG |
| | | | | ATTTCACTCTGACTATTAGCTCT |
| | | | | GTGCAGGCAGAAGACCTTGCCG |
| | | | | TGTATTACTGTCACCAGTATCT |
| | | | | GTCTTCAGACACGTTTGGAGGT |
| | | | | GGGACCAAACTAGAAATCAAA |
| | | | | CGTACTGTCGCA |
| Anti- | 166 | QVXLXQSGAELVRP | 180 | CAGGTGNNNCTGNNNCAGAGC |
| TGFb | | GTSVKVSCKASGYA | | GGCGCCGAGCTGGTGAGGCCCG |
| clone 1 | | FTNYLIEWVKQRPG | | GCACCAGCGTGAAGGTGAGCTG |
| VH | | QGLEWIGVNNPGSG | | CAAGGCCAGCGGCTACGCCTTC |
| | | GSNYNEKFKGKATL | | ACCAACTACCTGATCGAGTGGG |
| | | TADKSSSTAYMQLS | | TGAAGCAGAGGCCCGGCCAGG |
| | | SLTSDDSAVYFCAR | | GCCTGGAGTGGATCGGCGTGAA |
| | | SGGFYFDYWGQGT | | CAACCCCGGCAGCGGCGGCAG |
| | | TQSPSPQPKRRAH | | CAACTACAACGAGAAGTTCAAG |
| | | | | GGCAAGGCCACCCTGACCGCCG |
| | | | | ACAAGAGCAGCAGCACCGCCT |
| | | | | ACATGCAGCTGAGCAGCCTGAC |
| | | | | CAGCGACGACAGCGCCGTGTAC |
| | | | | TTCTGCGCCAGGAGCGGCGGCT |
| | | | | TCTACTTCGACTACTGGGGCCA |
| | | | | GGGCACCACCCAGAGCCCCAGC |
| | | | | CCCCAGCCCAAGAGGAGGGCC |
| | | | | CAC |
| Anti- | 167 | DIQMTQSPSSLSASV | 181 | GACATCCAGATGACCCAGAGCC |
| TGFb | | GDRVTITCRASQSV | | CCAGCAGCCTGAGCGCCAGCGT |
| clone 2 | | LYSSNQKNYLAWY | | GGGCGACAGGGTGACCATCACC |
| VL | | QQKPGKAPKLLIYW | | TGCAGGGCCAGCCAGAGCGTGC |
| | | ASTRESGVPSRFSGS | | TGTACAGCAGCAACCAGAAGA |
| | | GSGTDFTLTISSLQP | | ACTACCTGGCCTGGTACCAGCA |
| | | EDFATYYCHQYLSS | | GAAGCCCGGCAAGGCCCCCAA |
| | | DTFGQGTKVE | | GCTGCTGATCTACTGGGCCAGC |
| | | IKRTVA | | ACCAGGGAGAGCGGCGTGCCC |
| | | | | AGCAGGTTCAGCGGCAGCGGC |
| | | | | AGCGGCACCGACTTCACCCTGA |
| | | | | CCATCAGCAGCCTGCAGCCCGA |
| | | | | GGACTTCGCCACCTACTACTGC |
| | | | | CACCAGTACCTGAGCAGCGACA |
| | | | | CCTTCGGCCAGGGCACCAAGGT |
| | | | | GGAGATCAAGAGGACCGTGGCC |
| Anti- | 168 | EVQLVESGGGLVQP | 182 | GAGGTGCAGCTGGTGGAGAGC |
| TGFb | | GGSLRLSCAASGYA | | GGCGGCGGCCTGGTGCAGCCCG |
| clone 2 | | FTNYLIEWVRQAPG | | GCGGCAGCCTGAGGCTGAGCTG |
| VH | | KGLEWVGVNNPGS | | CGCCGCCAGCGGCTACGCCTTC |
| | | GGSNYNEKFKGRAT | | ACCAACTACCTGATCGAGTGGG |
| | | ISADNSKNTLYLQM | | TGAGGCAGGCCCCCGGCAAGG |
| | | NSLRAEDTAVYYCA | | GCCTGGAGTGGGTGGGCGTGAA |
| | | RSGGFYFDYWGQG | | CAACCCCGGCAGCGGCGGCAG |
| | | TLVTVSSASTKGPS | | CAACTACAACGAGAAGTTCAAG |
| | | | | GGCAGGGCCACCATCAGCGCCG |
| | | | | ACAACAGCAAGAACACCCTGTA |
| | | | | CCTGCAGATGAACAGCCTGAGG |
| | | | | GCCGAGGACACCGCCGTGTACT |
| | | | | ACTGCGCCAGGAGCGGCGGCTT |
| | | | | CTACTTCGACTACTGGGGCCAG |
| | | | | GGCACCCTGGTGACCGTGAGCA |
| | | | | GCGCCAGCACCAAGGGCCCCAGC |
| Anti- | 169 | LARPGASVKMSCKT | 183 | CTGGCCAGGCCCGGCGCCAGCG |
| TGFb | | SGYTFTNYWMHWV | | TGAAGATGAGCTGCAAGACCA |
| clone 3 | | RQRPGQGLEWIGTI | | GCGGCTACACCTTCACCAACTA |
| VH | | YPGNSDTNYNQKFK | | CTGGATGCACTGGGTGAGGCAG |
| | | DKAKLTAVTSATTA | | AGGCCCGGCCAGGGCCTGGAGT |
| | | YMELSSLTNEDSAV | | GGATCGGCACCATCTACCCCGG |
| | | YFCTREDSRSLYYN | | CAACAGCGACACCAACTACAAC |
| | | GWDYFDYWGQGTT | | CAGAAGTTCAAGGACAAGGCC |
| | | LTVSS | | AAGCTGACCGCCGTGACCAGCG |
| | | | | CCACCACCGCCTACATGGAGCT |
| | | | | GAGCAGCCTGACCAACGAGGA |
| | | | | CAGCGCCGTGTACTTCTGCACC |
| | | | | AGGGAGGACAGCAGGAGCCTG |
| | | | | TACTACAACGGCTGGGACTACT |
| | | | | TCGACTACTGGGGCCAGGGCAC |
| | | | | CACCCTGACCGTGAGCAGC |
| Anti- | 170 | LTQSPASLAVSLGQR | 184 | CTGACCCAGAGCCCCGCCAGCC |
| TGFb | | ATISCRASESVDNY | | TGGCCGTGAGCCTGGGCCAGAG |
| clone 3 | | GISFLNWFQQKPGQ | | GGCCACCATCAGCTGCAGGGCC |
| VL | | PPKLLIYAASNQGSG | | AGCGAGAGCGTGGACAACTAC |
| | | VPARFSGSGSGTDFS | | GGCATCAGCTTCCTGAACTGGT |
| | | LNIHPMEEDDTGMY | | TCCAGCAGAAGCCCGGCCAGCC |
| | | FCQQSKEVPRTFGG | | CCCCAAGCTGCTGATCTACGCC |
| | | GTKLEII | | GCCAGCAACCAGGGCAGCGGC |
| | | | | GTGCCCGCCAGGTTCAGCGGCA |
| | | | | GCGGCAGCGGCACCGACTTCAG |
| | | | | CCTGAACATCCACCCCATGGAG |
| | | | | GAGGACGACACCGGCATGTACT |
| | | | | TCTGCCAGCAGAGCAAGGAGGT |
| | | | | GCCCAGGACCTTCGGCGGCGGC |
| | | | | ACCAAGCTGGAGATCATC |
| Anti- | 171 | QVQLVQSGAEVKKP | 185 | CAGGTGCAGCTGGTGCAGAGCG |
| TGFb | | GSSVKVSCKASGGT | | GCGCCGAGGTGAAGAAGCCCG |
| clone 4 | | FSSYAISWVRQAPG | | GCAGCAGCGTGAAGGTGAGCT |
| VH | | QGLEWMGGIIPIFGT | | GCAAGGCCAGCGGCGGCACCTT |
| | | ANYAQKFQGRVTIT | | CAGCAGCTACGCCATCAGCTGG |
| | | ADESTSTAYMELSS | | GTGAGGCAGGCCCCCGGCCAG |
| | | LRSEDTAVYYCARG | | GGCCTGGAGTGGATGGGCGGC |
| | | LWEVRALPSVYWG | | ATCATCCCCATCTTCGGCACCG |
| | | QGTLVTVSS | | CCAACTACGCCCAGAAGTTCCA |
| | | | | GGGCAGGGTGACCATCACCGCC |
| | | | | GACGAGAGCACCAGCACCGCCT |
| | | | | ACATGGAGCTGAGCAGCCTGAG |
| | | | | GAGCGAGGACACCGCCGTGTAC |
| | | | | TACTGCGCCAGGGGCCTGTGGG |
| | | | | AGGTGAGGGCCCTGCCCAGCGT |
| | | | | GTACTGGGGCCAGGGCACCCTG |
| | | | | GTGACCGTGAGCAGC |
| Anti- | 172 | SYELTQPPSVSVAPG | 186 | AGCTACGAGCTGACCCAGCCCC |
| TGFb | | QTARITCGANDIGSK | | CCAGCGTGAGCGTGGCCCCCGG |
| clone 4 | | SVHWYQQKAGQAP | | CCAGACCGCCAGGATCACCTGC |
| VL | | VLVVSEDIIRPSGIPE | | GGCGCCAACGACATCGGCAGC |
| | | RISGSNSGNTATLTI | | AAGAGCGTGCACTGGTACCAGC |
| | | SRVEAGDEADYYC | | AGAAGGCCGGCCAGGCCCCCGT |
| | | QVWDRDSDQYVFG | | GCTGGTGGTGAGCGAGGACATC |
| | | TGTKVTVLG | | ATCAGGCCCAGCGGCATCCCCG |
| | | | | AGAGGATCAGCGGCAGCAACA |
| | | | | GCGGCAACACCGCCACCCTGAC |
| | | | | CATCAGCAGGGTGGAGGCCGG |
| | | | | CGACGAGGCCGACTACTACTGC |
| | | | | CAGGTGTGGGACAGGGACAGC |
| | | | | GACCAGTACGTGTTCGGCACCG |
| | | | | GCACCAAGGTGACCGTGCTGGGC |
| Anti- | 173 | QVQLVQSGAEVKKP | 187 | CAGGTGCAGCTGGTGCAGAGCG |
| TGFb | | GSSVKVSCKASGYT | | GCGCCGAGGTGAAGAAGCCCG |
| clone 5 | | FSSNVISWVRQAPG | | GCAGCAGCGTGAAGGTGAGCT |
| VH | | QGLEWMGGVIPIVD | | GCAAGGCCAGCGGCTACACCTT |
| | | IANYAQRFKGRVTIT | | CAGCAGCAACGTGATCAGCTGG |
| | | ADESTSTTYMELSSL | | GTGAGGCAGGCCCCCGGCCAG |
| | | RSEDTAVYYCASTL | | GGCCTGGAGTGGATGGGCGGC |
| | | GLVLDAMDYWGQG | | GTGATCCCCATCGTGGACATCG |
| | | TLVTVSS | | CCAACTACGCCCAGAGGTTCAA |
| | | | | GGGCAGGGTGACCATCACCGCC |
| | | | | GACGAGAGCACCAGCACCACCT |
| | | | | ACATGGAGCTGAGCAGCCTGAG |
| | | | | GAGCGAGGACACCGCCGTGTAC |
| | | | | TACTGCGCCAGCACCCTGGGCC |
| | | | | TGGTGCTGGACGCCATGGACTA |
| | | | | CTGGGGCCAGGGCACCCTGGTG |
| | | | | ACCGTGAGCAGC |
| Anti- | 174 | ETVLTQSPGTLSLSP | 188 | GAGACCGTGCTGACCCAGAGCC |
| TGFb | | GERATLSCRASQSL | | CCGGCACCCTGAGCCTGAGCCC |
| clone 5 | | GSSYLAWYQQKPG | | CGGCGAGAGGGCCACCCTGAG |
| VL | | QAPRLLIYGASSRAP | | CTGCAGGGCCAGCCAGAGCCTG |
| | | GIPDRFSGSGSGTDF | | GGCAGCAGCTACCTGGCCTGGT |
| | | TLTISRLEPEDFAVY | | ACCAGCAGAAGCCCGGCCAGG |
| | | YCQQYADSPITFGQ | | CCCCCAGGCTGCTGATCTACGG |
| | | GTRLEIK | | CGCCAGCAGCAGGGCCCCCGGC |
| | | | | ATCCCCGACAGGTTCAGCGGCA |
| | | | | GCGGCAGCGGCACCGACTTCAC |
| | | | | CCTGACCATCAGCAGGCTGGAG |
| | | | | CCCGAGGACTTCGCCGTGTACT |
| | | | | ACTGCCAGCAGTACGCCGACAG |
| | | | | CCCCATCACCTTCGGCCAGGGC |
| | | | | ACCAGGCTGGAGATCAAG |
| Anti- | 175 | QVQLVQSGAEVKKP | 189 | CAGGTGCAGCTGGTGCAGAGCG |
| TGFb | | GSSVKVSCKASGYT | | GCGCCGAGGTGAAGAAGCCCG |
| clone 6 | | FSSNVISWVRQAPG | | GCAGCAGCGTGAAGGTGAGCT |
| VH | | QGLEWMGGVIPIVD | | GCAAGGCCAGCGGCTACACCTT |
| | | IANYAQRFKGRVTIT | | CAGCAGCAACGTGATCAGCTGG |
| | | ADESTSTTYMELSSL | | GTGAGGCAGGCCCCCGGCCAG |
| | | RSEDTAVYYCALPR | | GGCCTGGAGTGGATGGGCGGC |
| | | AFVLDAMDYWGQG | | GTGATCCCCATCGTGGACATCG |
| | | TLVTVSS | | CCAACTACGCCCAGAGGTTCAA |
| | | | | GGGCAGGGTGACCATCACCGCC |
| | | | | GACGAGAGCACCAGCACCACCT |
| | | | | ACATGGAGCTGAGCAGCCTGAG |
| | | | | GAGCGAGGACACCGCCGTGTAC |
| | | | | TACTGCGCCCTGCCCAGGGCCT |
| | | | | TCGTGCTGGACGCCATGGACTA |
| | | | | CTGGGGCCAGGGCACCCTGGTG |
| | | | | ACCGTGAGCAGC |
| Anti- | 176 | ETVLTQSPGTLSLSP | 190 | GAGACCGTGCTGACCCAGAGCC |
| TGFb | | GERATLSCRASQSL | | CCGGCACCCTGAGCCTGAGCCC |
| clone 6 | | GSSYLAWYQQKPG | | CGGCGAGAGGGCCACCCTGAG |
| VL | | QAPRLLIYGASSRAP | | CTGCAGGGCCAGCCAGAGCCTG |
| | | GIPDRFSGSGSGTDF | | GGCAGCAGCTACCTGGCCTGGT |
| | | TLTISRLEPEDFAVY | | ACCAGCAGAAGCCCGGCCAGG |
| | | YCQQYADSPITFGQ | | CCCCCAGGCTGCTGATCTACGG |
| | | GTRLEIK | | CGCCAGCAGCAGGGCCCCCGGC |
| | | | | ATCCCCGACAGGTTCAGCGGCA |
| | | | | GCGGCAGCGGCACCGACTTCAC |
| | | | | CCTGACCATCAGCAGGCTGGAG |
| | | | | CCCGAGGACTTCGCCGTGTACT |
| | | | | ACTGCCAGCAGTACGCCGACAG |
| | | | | CCCCATCACCTTCGGCCAGGGC |
| | | | | ACCAGGCTGGAGATCAAG |
| Anti- | 177 | QVQLVQSGAEVKKP | 191 | CAGGTGCAGCTGGTGCAGAGCG |
| TGFb | | GSSVKVSCKASGGT | | GCGCCGAGGTGAAGAAGCCCG |
| clone 7 | | FSTSFINWVRQAPG | | GCAGCAGCGTGAAGGTGAGCT |
| VH | | QGLEWMGGIIPIFDI | | GCAAGGCCAGCGGCGGCACCTT |
| | | TNYAQKFQSRVTIT | | CAGCACCAGCTTCATCAACTGG |
| | | ADKSTSTAYMELSS | | GTGAGGCAGGCCCCCGGCCAG |
| | | LRSEDTAVYYCARG | | GGCCTGGAGTGGATGGGCGGC |
| | | NGNYALDAMDYW | | ATCATCCCCATCTTCGACATCA |
| | | GQGTLVTVSS | | CCAACTACGCCCAGAAGTTCCA |
| | | | | GAGCAGGGTGACCATCACCGCC |
| | | | | GACAAGAGCACCAGCACCGCCT |
| | | | | ACATGGAGCTGAGCAGCCTGAG |
| | | | | GAGCGAGGACACCGCCGTGTAC |
| | | | | TACTGCGCCAGGGGCAACGGCA |
| | | | | ACTACGCCCTGGACGCCATGGA |
| | | | | CTACTGGGGCCAGGGCACCCTG |
| | | | | GTGACCGTGAGCAGC |
| Anti- | 178 | EIVLTQSPGTLSLSPG | 192 | GAGATCGTGCTGACCCAGAGCC |
| TGFb | | ERATLSCRASQSVSS | | CCGGCACCCTGAGCCTGAGCCC |
| clone 7 | | SYFAWYQQKPGQA | | CGGCGAGAGGGCCACCCTGAG |
| VL | | PRLLIYGASSRATGI | | CTGCAGGGCCAGCCAGAGCGTG |
| | | PDRFSGSGSGTDFTL | | AGCAGCAGCTACTTCGCCTGGT |
| | | TISRLEPEDFAVYYC | | ACCAGCAGAAGCCCGGCCAGG |
| | | QQYYDSPITFGQGT | | CCCCCAGGCTGCTGATCTACGG |
| | | RLEIK | | CGCCAGCAGCAGGGCCACCGG |
| | | | | CATCCCCGACAGGTTCAGCGGC |
| | | | | AGCGGCAGCGGCACCGACTTCA |
| | | | | CCCTGACCATCAGCAGGCTGGA |
| | | | | GCCCGAGGACTTCGCCGTGTAC |
| | | | | TACTGCCAGCAGTACTACGACA |
| | | | | GCCCCATCACCTTCGGCCAGGG |
| | | | | CACCAGGCTGGAGATCAAG |

### Table 15. Exemplary TGFβ1 Inhibitory Peptide and Nucleotide Sequences

Table 15 provides exemplary TGFβ1 inhibitory peptides for use in anti-PD-1 fusion proteins described herein. In any embodiments exemplifying a fusion protein with, e.g., a TGFβRII ECD, TGFβ1 inhibitory peptides may be employed instead.

| | SEQ ID | Amino acid | SEQ ID | Nucleotide |
|---|---|---|---|---|
| Name | NO | sequence | NO | sequence |
| TGF | 263 | HANFCLGPCPYIW | 268 | CACGCCAACTTCTGCCTGGGCCCCT |
| b1- | | SLA | | GCCCCTACATCTGGAGCCTGGCC |
| I36 | | | | |
| TGF | 264 | FCLGPCPYIWSLD | 269 | TTCTGCCTGGGCCCCTGCCCCTACA |
| b1- | | TA | | TCTGGAGCCTGGACACCGCC |
| I37 | | | | |
| TGF | 265 | SNPYSAFQVDIIV | 270 | AGCAACCCCTACAGCGCCTTCCAGG |
| b1- | | DIA | | TGGACATCATCGTGGACATCGCC |
| I38 | | | | |
| TGF | 266 | TSLDATMIWTMMA | 271 | ACCAGCCTGGACGCCACCATGATCT |
| b1- | | | | GGACCATGATGGCC |
| I39 | | | | |
| TGF | 267 | TSLDASIWAMMQN | 272 | ACCAGCCTGGACGCCAGCATCTGGG |
| b1- | | A | | CCATGATGCAGAACGCC |
| I40 | | | | |

### Table 16. Exemplary Fhuman ADA2 Sequences

Table 16 provides exemplary ADA2 sequences for use in anti-PD-1 fusion proteins described herein. In any embodiments exemplifying a fusion protein with, e.g., a TGFβRII ECD, an ADA2 sequence may be employed instead.

| | SEQ | | SEQ | |
|---|---|---|---|---|
| | ID | Amino Acid | ID | NUCLEOTIDE |
| Name | NO | Sequence | NO | SEQUENCE |
| ADA2 | 273 | IDETRAHLLLKEKM | | |
| mutant 1 | | MRLGGRLVLNTKEE | | |
| | | LANERLMTLKIAEM | | |
| | | KEAMRTLIFPPSMH | | |
| | | FFQAKHLIERSQVF | | |
| | | NILRMMPKGAALHL | | |
| | | HDIGIVTMDWLVRN | | |
| | | VTYRPHGIALPGDS | | |
| | | LLRNFTLVTQHPEV | | |
| | | IYTNQNVVLSKFET | | |
| | | IFFTISGLIHYAPV | | |
| | | FRDYVFRSMQEFYE | | |
| | | DNVLYMEIRASLLP | | |
| | | VYELSGEHHDEEWS | | |
| | | VKTYQEVAQDFVET | | |
| | | HPEFIGIKIIYSDH | | |
| | | RSKDVAVIAESIRM | | |
| | | AMGLRIKFPTVVAG | | |
| | | FDLVGHEDTGHSLH | | |
| | | DYKEALMIPAKDGV | | |
| | | KLPYFFHAGETDWQ | | |
| | | GTSIDRNILDALML | | |
| | | NTTRIGHGFALSKH | | |
| | | PAVRTYSWKKDIPI | | |
| | | EVCPISNQVLKLVS | | |
| | | DLRNHPVATLMATG | | |
| | | HPMVISSDDPAMFG | | |
| | | AKGLSYDFYEVFMG | | |
| | | IGGMKADLRTLKQL | | |
| | | AMNSIKYSTLLESE | | |
| | | KNTFMEIWKKRWDK | | |
| | | FIADVATK | | |
| ADA2 | 274 | IDETRAHLLLKEKM | | |
| mutant 2 | | MRLGGRLVLNTKEE | | |
| | | LANERLMTLKIAEM | | |
| | | KEAMRTLIFPPSMH | | |
| | | FFQAKHLIERSQVF | | |
| | | NILRMMPKGAALHL | | |
| | | HDIGIVTMDWLVRN | | |
| | | VTYRPHGIALPGDS | | |
| | | LLRNFTLVTQHPEV | | |
| | | IYTNQNVVWSKFET | | |
| | | IVFTISGLIHYAPV | | |
| | | FRDYVFRSMQEFYE | | |
| | | DNVLYMEIRARLLP | | |
| | | VYELSGEHHDEEWS | | |
| | | VKTYQEVAQDFVET | | |
| | | HPEFIGIKIIYSDH | | |
| | | RSKDVAVIAESIRM | | |
| | | AMGLRIKFPTVVAG | | |
| | | FDLAGHEDTGHSLH | | |
| | | DYKEALMIPAKDGV | | |
| | | KLPYFFHAGETDWQ | | |
| | | GTSIDRNILDALML | | |
| | | NTTRIGHGFALSKH | | |
| | | PAVRTYSWKKDIPI | | |
| | | EVCPISNQVLKLVS | | |
| | | DLRNHPVATLMATG | | |
| | | HPMVISSDDPAMFG | | |
| | | AKGLSYDFYEVFMG | | |
| | | IGGMKADLRTLKQL | | |
| | | AMNSIKYSTLLESE | | |
| | | KNTFMEIWKKRWDK | | |
| | | FIADVATK | | |
| ADA2 | 275 | EMKEAMRTLIFPPS | | |
| mutant 3 | | MHFFQAKHLIERSQ | | |
| | | VFNILRMMPKGAAL | | |
| | | HLHDIGIVTMDWLV | | |
| | | RNVTYRPHGIALPG | | |
| | | DSLLRNFTLVTQHP | | |
| | | EVIYTNQNVVLSKF | | |
| | | ETIVFTISGLIHYA | | |
| | | PVFRDYVFRSMQEF | | |
| | | YEDNVLYMEIRARL | | |
| | | LPVYELSGEHHDEE | | |
| | | WSVKTYQEVAQDFV | | |
| | | ETHPEFIGIKIIYS | | |
| | | DHRSKDVAVIAESI | | |
| | | RMAMGLRIKFPTVV | | |
| | | AGFDLVGHEDTGHS | | |
| | | LHDYKEALMIPAKD | | |
| | | GVKLPYFFHAGETD | | |
| | | WQGTSIDRNILDAL | | |
| | | MLNTTRIGHGFALS | | |
| | | KHPAVRTYSWKKDI | | |
| | | PIEVCPISNQVLKL | | |
| | | VSDLRNHPVATLMA | | |
| | | TGHPMVISSDDPAM | | |
| | | FGAKGLSYDFYEVF | | |
| | | MGIGGMKADLRTLK | | |
| | | QLAMNSIKYSTLLE | | |
| | | SEKNTFMEIWKKRW | | |
| | | DKFIADVATK | | |
| ADA2 | 276 | IDETRAHLLLKEKM | | |
| mutant 4 | | MRLGGRLVLNTKEE | | |
| | | LANERLMTLKIAEM | | |
| | | KEAMRTLIFPPSMH | | |
| | | FFQAKHLIERSQVF | | |
| | | NILRMMPKGAALHL | | |
| | | HDIGIVTMDWLGGG | | |
| | | GSGGGGSVTEFDDS | | |
| | | LLRNFTLVTQHPEV | | |
| | | IYTNQNVVWSKFET | | |
| | | IFFTISGLIHYAPV | | |
| | | FRDYVFRSMQEFYE | | |
| | | DNVLYMEIRARLLP | | |
| | | VYELSGEHHDEEWS | | |
| | | VKTYQEVAQEFVET | | |
| | | HPEFIGIKIIYSDH | | |
| | | RSRDVAVIAESIRM | | |
| | | AMGLRIKFPTVVAG | | |
| | | FDLSGHEDTGHSLH | | |
| | | DYKEALMIPAKDGV | | |
| | | KLPYFFHAGETDWQ | | |
| | | GTSIDRNILDALML | | |
| | | NTTRIGHGFALSKH | | |
| | | PAVRTYSWKKDIPI | | |
| | | EVCPISNQVLKLVS | | |
| | | DLRNHPVATLMATG | | |
| | | HPMVISSDDPAMFG | | |
| | | AKGLSYDFYEVFMG | | |
| | | IGGMKADLRTLKQL | | |
| | | AMNSIKYSTLLESE | | |
| | | KNTFMEIWKKRWDK | | |
| | | FIADVATK | | |
| ADA2 | 277 | IDETRAHLLLKEKM | | |
| mutant 5 | | MRLGGRLVLNTKEE | | |
| | | LANERLMTLKIAEM | | |
| | | KEAMRTLIFPPSMH | | |
| | | FFQAKHLIERSQVF | | |
| | | NILRMMPKGAALHL | | |
| | | HNIGIVTMDWLGGG | | |
| | | GSGGGGSVTEFDDS | | |
| | | LLRNFTLVTQHPEV | | |
| | | IYTNQNVVWSKFET | | |
| | | IFFTISGLIHYAPV | | |
| | | FRDYVFRSMQEFYE | | |
| | | DNVLYMEIRARLLP | | |
| | | VYELSGEHHDEEWS | | |
| | | VKTYQEVAQDFVET | | |
| | | HPEFIGIKIIYSDH | | |
| | | RSYDVAVIAESIRM | | |
| | | AMGLRIKFPTVVAG | | |
| | | FDLVGHEDTGHSLH | | |
| | | DYKEALMIPAKDGV | | |
| | | KLPYFFHAGETDWQ | | |
| | | GTSIDRNILDALML | | |
| | | NTTRIGHGFALSKH | | |
| | | PAVRTYSWKKDIPI | | |
| | | EVCPISNQVLKLVS | | |
| | | DLRNHPVATLMATG | | |
| | | HPMVISSDDPAMFG | | |
| | | AKGLSYDFYEVFMG | | |
| | | IGGMKADLRTLKQL | | |
| | | AMNSIKYSTLLESE | | |
| | | KNTFMEIWKKRWDK | | |
| | | FIADVATK | | |
| ADA2 | 278 | IDETRAHLLLKEKM | | |
| mutant 6 | | MRLGGRLVLNTKEE | | |
| | | LANERLMTLKIAEM | | |
| | | KEAMRTLIFPPSMH | | |
| | | FFQAKHLIERSQVF | | |
| | | NILRMMPKGAALHL | | |
| | | HDIGIVTMDWLVRN | | |
| | | VTYRPHCHICFTPR | | |
| | | GIMQFRFAHPTPRP | | |
| | | SEKCSKWILLEDYR | | |
| | | KRVQNVTEFDDSLL | | |
| | | RNFTLVTQHPEVIY | | |
| | | TNQNVVWSKFETIF | | |
| | | FTISGLIHYAPVFR | | |
| | | DYVFRSMQEFYEDN | | |
| | | VLYMEIRARLLPVY | | |
| | | ELSGEHHDEEWSVK | | |
| | | TYQEVAQKFVETHP | | |
| | | EFIGIKIIYSDHRS | | |
| | | KDVAVIAESIRMAM | | |
| | | GLRIKFPTVVAGFD | | |
| | | LVGHEDTGHSLHDY | | |
| | | KEALMIPAKDGVKL | | |
| | | PYFFHAGETDWQGT | | |
| | | SIDRNILDALMLNT | | |
| | | TRIGHGFALSKHPA | | |
| | | VRTYSWKKDIPIEV | | |
| | | CPISNQVLKLVSDL | | |
| | | RNHPVATLMATGHP | | |
| | | MVISSDDPAMFGAK | | |
| | | GLSYDFYEVFMGIG | | |
| | | GMKADLRTLKQLAM | | |
| | | NSIKYSTLLESEKN | | |
| | | TFMEIWKKRWDKFI | | |
| | | ADVATK | | |
| ADA2 | 279 | IDETRAHLLLKEKM | | |
| mutant 7 | | MRLGGRLVLNTKEE | | |
| | | LANERLMTLKIAEM | | |
| | | KEAMRTLIFPPSMH | | |
| | | FFQAKHLIERSQVE | | |
| | | NILRMMPKGAALHL | | |
| | | HDIGIVTMDWLVRN | | |
| | | VTYRPHCHICFTPR | | |
| | | GIMQFRFAHPTPRP | | |
| | | SEKCSKWILLEDYR | | |
| | | KRVQNVTEFDDSLL | | |
| | | RNFTLVTQHPEVIY | | |
| | | TNQNVVWSKFETIF | | |
| | | FTISGLIHYAPVFR | | |
| | | DYVFRSMQEFYEDN | | |
| | | VLYMEIRAQLLPVY | | |
| | | ELSGEHHDEEWSVK | | |
| | | TYQEVAQKFVETHP | | |
| | | EFIGIKIIYNDHRS | | |
| | | KDVAVIAESIRMAM | | |
| | | GLRIKFPTVVAGFD | | |
| | | LVGHEDTGHSLHDY | | |
| | | KEALMIPAKDGVKL | | |
| | | PYFFHAGETDWQGT | | |
| | | SIDRNILDALMLNT | | |
| | | TRIGHGFALSKHPA | | |
| | | VRTYSWDKDIPIEV | | |
| | | CPISNQVLKLVSDL | | |
| | | RNHPVATLMATGHP | | |
| | | MVISSDDPAMFGAK | | |
| | | GLSYDFYEVFMGIG | | |
| | | GMKADLRTLKQLAM | | |
| | | NSIKYSTLLESEKN | | |
| | | TFMEIWKKRWDKFI | | |
| | | ADVATK | | |
| VH6 | 280 | QVQLVESGGGVVQP | 285 | CAGGTGCAGCTGGTCGAAAGCGGAGG |
| ADA2 | | GRSLRLDCKASGIT | | AGGAGTGGTCCAGCCAGGACGATCCC |
| | | FSNSGMHWVRQAPG | | TGAGACTGGATTGTAAGGCCTCTGGA |
| | | KGLEWVAVIWYDGS | | ATCACATTCTCTAACAGTGGAATGCA |
| | | KRYYADSVKGRFTI | | CTGGGTGCGCCAGGCACCAGGAAAAG |
| | | SRDNSKNTLFLQMN | | GACTGGAGTGGGTGGCCGTCATCTGG |
| | | SLRAEDTAVYYCAT | | TACGACGGGTCAAAGCGATACTATGC |
| | | NDDYWGQGTLVTVS | | AGATAGCGTGAAAGGAAGGTTCACAA |
| | | SASTKGPSVFPLAP | | TTTCACGCGACAACAGCAAGAATACT |
| | | CSRSTSESTAALGC | | CTGTTTCTGCAGATGAACTCTCTGAG |
| | | LVKDYFPEPVTVSW | | AGCAGAGGATACTGCCGTGTACTATT |
| | | NSGALTSGVHTFPA | | GTGCTACCAATGACGATTATTGGGGG |
| | | VLQSSGLYSLSSVV | | CAGGGAACTCTGGTGACCGTCAGTTC |
| | | TVPSSSLGTKTYTC | | AGCTAGCACCAAGGGCCCATCGGTCT |
| | | NVDHKPSNTKVDKR | | TCCCCCTGGCGCCCTGCTCCAGGAGC |
| | | VESKYGPPCPPCPA | | ACCTCCGAGAGCACAGCCGCCCTGGG |
| | | PEFLGGPSVFLFPP | | CTGCCTGGTCAAGGACTACTTCCCCG |
| | | KPKDTLMISRTPEV | | AACCGGTGACGGTGTCGTGGAACTCA |
| | | TCVVVDVSQEDPEV | | GGCGCCCTGACCAGCGGCGTGCACAC |
| | | QFNWYVDGVEVHNA | | CTTCCCGGCTGTCCTACAGTCCTCAG |
| | | KTKPREEQFNSTYR | | GACTCTACTCCCTCAGCAGCGTGGTG |
| | | VVSVLTVLHQDWLN | | ACCGTGCCCTCCAGCAGCTTGGGCAC |
| | | GKEYKCKVSNKGLP | | GAAGACCTACACCTGCAACGTAGATC |
| | | SSIEKTISKAKGQP | | ACAAGCCCAGCAACACCAAGGTGGAC |
| | | REPQVYTLPPSQEE | | AAGAGAGTTGAGTCCAAATATGGTCC |
| | | MTKNQVSLTCLVKG | | CCCATGCCCACCATGCCCAGCACCTG |
| | | FYPSDIAVEWESNG | | AGTTCCTGGGGGGACCATCAGTCTTC |
| | | QPENNYKTTPPVLD | | CTGTTCCCCCCAAAACCCAAGGACAC |
| | | SDGSFFLYSRLTVD | | TCTCATGATCTCCCGGACCCCTGAGG |
| | | KSRWQEGNVFSCSV | | TCACGTGCGTGGTGGTGGACGTGAGC |
| | | MHEALHNHYTQKSL | | CAGGAAGACCCCGAGGTCCAGTTCAA |
| | | SLSPGKGGGGSGGG | | CTGGTACGTGGATGGCGTGGAGGTGC |
| | | GSIDETRAHLLLKE | | ATAATGCCAAGACAAAGCCGCGGGAG |
| | | KMMRLGGRLVLNTK | | GAGCAGTTCAACAGCACGTACCGTGT |
| | | EELANERLMTLKIA | | GGTCAGCGTCCTCACCGTCCTGCACC |
| | | EMKEAMRTLIFPPS | | AGGACTGGCTGAACGGCAAGGAGTAC |
| | | MHFFQAKHLIERSQ | | AAGTGCAAGGTCTCCAACAAAGGCCT |
| | | VFNILRMMPKGAAL | | CCCGTCCTCCATCGAGAAAACCATCT |
| | | HLHDIGIVTMDWLV | | CCAAAGCCAAAGGGCAGCCCCGAGAG |
| | | RNVTYRPHCHICFT | | CCACAGGTGTACACCCTGCCCCCATC |
| | | PRGIMQFRFAHPTP | | CCAGGAGGAGATGACCAAGAACCAGG |
| | | RPSEKCSKWILLED | | TCAGCCTGACCTGCCTGGTCAAAGGC |
| | | YRKRVQNVTEFDDS | | TTCTACCCCAGCGACATCGCCGTGGA |
| | | LLRNFTLVTQHPEV | | GTGGGAGAGCAATGGGCAGCCGGAGA |
| | | IYTNQNVVWSKFET | | ACAACTACAAGACCACGCCTCCCGTG |
| | | IFFTISGLIHYAPV | | CTGGACTCCGACGGCTCCTTCTTCCT |
| | | FRDYVFRSMQEFYE | | CTACAGCAGGCTCACCGTGGACAAGA |
| | | DNVLYMEIRARLLP | | GCAGGTGGCAGGAGGGGAATGTCTTC |
| | | VYELSGEHHDEEWS | | TCATGCTCCGTGATGCATGAGGCTCT |
| | | VKTYQEVAQKFVET | | GCACAACCACTACACACAGAAGAGCC |
| | | HPEFIGIKIIYSDH | | TCTCCCTGTCTCCGGGTAAAGGTGGA |
| | | RSKDVAVIAESIRM | | GGTGGTTCTGGAGGTGGAGGTAGTAT |
| | | AMGLRIKFPTVVAG | | CGACGAAACCAGAGCACACTTACTGC |
| | | FDLVGHEDTGHSLH | | TGAAAGAGAAAATGATGCGCCTGGGC |
| | | DYKEALMIPAKDGV | | GGGAGATTGGTGTTAAATACTAAGGA |
| | | KLPYFFHAGETDWQ | | AGAGCTGGCAAATGAAAGACTCATGA |
| | | GTSIDRNILDALML | | CACTGAAGATTGCTGAAATGAAGGAG |
| | | NTTRIGHGFALSKH | | GCGATGAGGACGCTGATCTTTCCGCC |
| | | PAVRTYSWKKDIPI | | TTCCATGCACTTCTTCCAAGCTAAAC |
| | | EVCPISNQVLKLVS | | ACCTGATCGAAAGATCCCAAGTGTTT |
| | | DLRNHPVATLMATG | | AACATCCTGAGGATGATGCCTAAGGG |
| | | HPMVISSDDPAMFG | | GGCCGCTCTGCACCTTCACGATATTG |
| | | AKGLSYDFYEVFMG | | GGATTGTAACAATGGACTGGCTGGTA |
| | | IGGMKADLRTLKQL | | AGGAACGTGACATACAGACCTCATTG |
| | | AMNSIKYSTLLESE | | CCATATTTGTTTTACTCCCCGAGGAA |
| | | KNTFMEIWKKRWDK | | TCATGCAATTCAGGTTTGCCCACCCA |
| | | FIADVATK | | ACTCCTCGGCCAAGCGAGAAGTGTAG |
| | | | | TAAGTGGATTTTGCTGGAAGATTACC |
| | | | | GTAAGCGCGTGCAGAATGTGACAGAG |
| | | | | TTTGATGACTCCCTGCTCCGCAATTT |
| | | | | TACCCTGGTGACCCAGCACCCCGAAG |
| | | | | TTATATACACTAACCAAAATGTCGTG |
| | | | | TGGTCCAAGTTTGAGACGATCTTCTT |
| | | | | CACGATTTCAGGCTTGATCCACTACG |
| | | | | CCCCGGTCTTTCGGGATTATGTGTTT |
| | | | | AGGAGTATGCAGGAGTTTTATGAGGA |
| | | | | TAATGTTCTGTACATGGAGATCCGAG |
| | | | | CCCGGCTGCTTCCAGTCTACGAACTA |
| | | | | TCCGGCGAACACCATGACGAGGAATG |
| | | | | GAGCGTCAAGACCTATCAAGAGGTGG |
| | | | | CCCAGAAGTTCGTAGAAACGCATCCA |
| | | | | GAGTTCATCGGTATTAAGATTATCTA |
| | | | | CTCTGATCACCGCTCAAAGGATGTGG |
| | | | | CTGTCATCGCCGAGTCTATACGGATG |
| | | | | GCCATGGGCCTGCGGATTAAGTTCCC |
| | | | | TACCGTCGTCGCCGGATTCGACCTCG |
| | | | | TTGGGCATGAGGATACTGGCCATAGT |
| | | | | CTCCATGACTATAAAGAAGCCCTTAT |
| | | | | GATCCCAGCAAAGGACGGAGTGAAGC |
| | | | | TGCCCTACTTCTTCCACGCAGGGGAG |
| | | | | ACCGACTGGCAGGGAACGAGCATCGA |
| | | | | CCGGAACATACTTGATGCACTCATGC |
| | | | | TTAATACCACACGAATCGGCCACGGC |
| | | | | TTCGCTCTCTCCAAGCACCCAGCCGT |
| | | | | GAGAACCTACAGCTGGAAGAAGGATA |
| | | | | TCCCCATCGAGGTTTGTCCCATCAGC |
| | | | | AATCAGGTGCTGAAATTGGTGAGTGA |
| | | | | CCTGAGAAACCACCCAGTCGCAACAT |
| | | | | TAATGGCCACTGGCCACCCTATGGTG |
| | | | | ATTTCAAGCGATGATCCAGCCATGTT |
| | | | | CGGAGCAAAAGGACTCAGTTACGACT |
| | | | | TCTATGAGGTATTCATGGGTATTGGT |
| | | | | GGTATGAAGGCAGACCTGCGGACTCT |
| | | | | TAAGCAGTTGGCAATGAACTCAATTA |
| | | | | AGTACTCTACCTTATTGGAGTCTGAA |
| | | | | AAGAACACATTTATGGAGATCTGGAA |
| | | | | AAAGCGCTGGGACAAATTCATCGCAG |
| | | | | ATGTTGCCACAAAA |
| VH6 | 281 | QVQLVESGGGVVQP | 286 | CAGGTGCAGCTGGTCGAAAGCGGAGG |
| ADA2 | | GRSLRLDCKASGIT | | AGGAGTGGTCCAGCCAGGACGATCCC |
| mut 7 | | FSNSGMHWVRQAPG | | TGAGACTGGATTGTAAGGCCTCTGGA |
| | | KGLEWVAVIWYDGS | | ATCACATTCTCTAACAGTGGAATGCA |
| | | KRYYADSVKGRFTI | | CTGGGTGCGCCAGGCACCAGGAAAAG |
| | | SRDNSKNTLFLQMN | | GACTGGAGTGGGTGGCCGTCATCTGG |
| | | SLRAEDTAVYYCAT | | TACGACGGGTCAAAGCGATACTATGC |
| | | NDDYWGQGTLVTVS | | AGATAGCGTGAAAGGAAGGTTCACAA |
| | | SASTKGPSVFPLAP | | TTTCACGCGACAACAGCAAGAATACT |
| | | CSRSTSESTAALGC | | CTGTTTCTGCAGATGAACTCTCTGAG |
| | | LVKDYFPEPVTVSW | | AGCAGAGGATACTGCCGTGTACTATT |
| | | NSGALTSGVHTFPA | | GTGCTACCAATGACGATTATTGGGGG |
| | | VLQSSGLYSLSSVV | | CAGGGAACTCTGGTGACCGTCAGTTC |
| | | TVPSSSLGTKTYTC | | AGCTAGCACCAAGGGCCCATCGGTCT |
| | | NVDHKPSNTKVDKR | | TCCCCCTGGCGCCCTGCTCCAGGAGC |
| | | VESKYGPPCPPCPA | | ACCTCCGAGAGCACAGCCGCCCTGGG |
| | | PEFLGGPSVFLFPP | | CTGCCTGGTCAAGGACTACTTCCCCG |
| | | KPKDTLMISRTPEV | | AACCGGTGACGGTGTCGTGGAACTCA |
| | | TCVVVDVSQEDPEV | | GGCGCCCTGACCAGCGGCGTGCACAC |
| | | QFNWYVDGVEVHNA | | CTTCCCGGCTGTCCTACAGTCCTCAG |
| | | KTKPREEQFNSTYR | | GACTCTACTCCCTCAGCAGCGTGGTG |
| | | VVSVLTVLHQDWLN | | ACCGTGCCCTCCAGCAGCTTGGGCAC |
| | | GKEYKCKVSNKGLP | | GAAGACCTACACCTGCAACGTAGATC |
| | | SSIEKTISKAKGQP | | ACAAGCCCAGCAACACCAAGGTGGAC |
| | | REPQVYTLPPSQEE | | AAGAGAGTTGAGTCCAAATATGGTCC |
| | | MTKNQVSLTCLVKG | | CCCATGCCCACCATGCCCAGCACCTG |
| | | FYPSDIAVEWESNG | | AGTTCCTGGGGGGACCATCAGTCTTC |
| | | QPENNYKTTPPVLD | | CTGTTCCCCCCAAAACCCAAGGACAC |
| | | SDGSFFLYSRLTVD | | TCTCATGATCTCCCGGACCCCTGAGG |
| | | KSRWQEGNVFSCSV | | TCACGTGCGTGGTGGTGGACGTGAGC |
| | | MHEALHNHYTOKSL | | CAGGAAGACCCCGAGGTCCAGTTCAA |
| | | SLSPGKGGGGSGGG | | CTGGTACGTGGATGGCGTGGAGGTGC |
| | | GSIDETRAHLLLKE | | ATAATGCCAAGACAAAGCCGCGGGAG |
| | | KMMRLGGRLVLNTK | | GAGCAGTTCAACAGCACGTACCGTGT |
| | | EELANERLMTLKIA | | GGTCAGCGTCCTCACCGTCCTGCACC |
| | | EMKEAMRTLIFPPS | | AGGACTGGCTGAACGGCAAGGAGTAC |
| | | MHFFQAKHLIERSQ | | AAGTGCAAGGTCTCCAACAAAGGCCT |
| | | VFNILRMMPKGAAL | | CCCGTCCTCCATCGAGAAAACCATCT |
| | | HLHDIGIVTMDWLV | | CCAAAGCCAAAGGGCAGCCCCGAGAG |
| | | RNVTYRPHCHICFT | | CCACAGGTGTACACCCTGCCCCCATC |
| | | PRGIMQFRFAHPTP | | CCAGGAGGAGATGACCAAGAACCAGG |
| | | RPSEKCSKWILLED | | TCAGCCTGACCTGCCTGGTCAAAGGC |
| | | YRKRVQNVTEFDDS | | TTCTACCCCAGCGACATCGCCGTGGA |
| | | LLRNFTLVTQHPEV | | GTGGGAGAGCAATGGGCAGCCGGAGA |
| | | IYTNQNVVWSKFET | | ACAACTACAAGACCACGCCTCCCGTG |
| | | IFFTISGLIHYAPV | | CTGGACTCCGACGGCTCCTTCTTCCT |
| | | FRDYVFRSMQEFYE | | CTACAGCAGGCTCACCGTGGACAAGA |
| | | DNVLYMEIRAQLLP | | GCAGGTGGCAGGAGGGGAATGTCTTC |
| | | VYELSGEHHDEEWS | | TCATGCTCCGTGATGCATGAGGCTCT |
| | | VKTYQEVAQKFVET | | GCACAACCACTACACACAGAAGAGCC |
| | | HPEFIGIKIIYNDH | | TCTCCCTGTCTCCGGGTAAAGGTGGA |
| | | RSKDVAVIAESIRM | | GGTGGTTCTGGAGGTGGAGGTAGTAT |
| | | AMGLRIKFPTVVAG | | CGACGAAACCAGAGCACACTTACTGC |
| | | FDLVGHEDTGHSLH | | TGAAAGAGAAAATGATGCGCCTGGGC |
| | | DYKEALMIPAKDGV | | GGGAGATTGGTGTTAAATACTAAGGA |
| | | KLPYFFHAGETDWQ | | AGAGCTGGCAAATGAAAGACTCATGA |
| | | GTSIDRNILDALML | | CACTGAAGATTGCTGAAATGAAGGAG |
| | | NTTRIGHGFALSKH | | GCGATGAGGACGCTGATCTTTCCGCC |
| | | PAVRTYSWDKDIPI | | TTCCATGCACTTCTTCCAAGCTAAAC |
| | | EVCPISNQVLKLVS | | ACCTGATCGAAAGATCCCAAGTGTTT |
| | | DLRNHPVATLMATG | | AACATCCTGAGGATGATGCCTAAGGG |
| | | HPMVISSDDPAMFG | | GGCCGCTCTGCACCTTCACGATATTG |
| | | AKGLSYDFYEVFMG | | GGATTGTAACAATGGACTGGCTGGTA |
| | | IGGMKADLRTLKQL | | AGGAACGTGACATACAGACCTCATTG |
| | | AMNSIKYSTLLESE | | CCATATTTGTTTTACTCCCCGAGGAA |
| | | KNTFMEIWKKRWDK | | TCATGCAATTCAGGTTTGCCCACCCA |
| | | FIADVATK | | ACTCCTCGGCCAAGCGAGAAGTGTAG |
| | | | | TAAGTGGATTTTGCTGGAAGATTACC |
| | | | | GTAAGCGCGTGCAGAATGTGACAGAG |
| | | | | TTTGATGACTCCCTGCTCCGCAATTT |
| | | | | TACCCTGGTGACCCAGCACCCCGAAG |
| | | | | TTATATACACTAACCAAAATGTCGTG |
| | | | | TGGTCCAAGTTTGAGACGATCTTCTT |
| | | | | CACGATTTCAGGCTTGATCCACTACG |
| | | | | CCCCGGTCTTTCGGGATTATGTGTTT |
| | | | | AGGAGTATGCAGGAGTTTTATGAGGA |
| | | | | TAATGTTCTGTACATGGAGATCCGAG |
| | | | | CCCAGCTGCTTCCAGTCTACGAACTA |
| | | | | TCCGGCGAACACCATGACGAGGAATG |
| | | | | GAGCGTCAAGACCTATCAAGAGGTGG |
| | | | | CCCAGAAGTTCGTAGAAACGCATCCA |
| | | | | GAGTTCATCGGTATTAAGATTATCTA |
| | | | | CAATGATCACCGCTCAAAGGATGTGG |
| | | | | CTGTCATCGCCGAGTCTATACGGATG |
| | | | | GCCATGGGCCTGCGGATTAAGTTCCC |
| | | | | TACCGTCGTCGCCGGATTCGACCTCG |
| | | | | TTGGGCATGAGGATACTGGCCATAGT |
| | | | | CTCCATGACTATAAAGAAGCCCTTAT |
| | | | | GATCCCAGCAAAGGACGGAGTGAAGC |
| | | | | TGCCCTACTTCTTCCACGCAGGGGAG |
| | | | | ACCGACTGGCAGGGAACGAGCATCGA |
| | | | | CCGGAACATACTTGATGCACTCATGC |
| | | | | TTAATACCACACGAATCGGCCACGGC |
| | | | | TTCGCTCTCTCCAAGCACCCAGCCGT |
| | | | | GAGAACCTACAGCTGGGATAAGGATA |
| | | | | TCCCCATCGAGGTTTGTCCCATCAGC |
| | | | | AATCAGGTGCTGAAATTGGTGAGTGA |
| | | | | CCTGAGAAACCACCCAGTCGCAACAT |
| | | | | TAATGGCCACTGGCCACCCTATGGTG |
| | | | | ATTTCAAGCGATGATCCAGCCATGTT |
| | | | | CGGAGCAAAAGGACTCAGTTACGACT |
| | | | | TCTATGAGGTATTCATGGGTATTGGT |
| | | | | GGTATGAAGGCAGACCTGCGGACTCT |
| | | | | TAAGCAGTTGGCAATGAACTCAATTA |
| | | | | AGTACTCTACCTTATTGGAGTCTGAA |
| | | | | AAGAACACATTTATGGAGATCTGGAA |
| | | | | AAAGCGCTGGGACAAATTCATCGCAG |
| | | | | ATGTTGCCACAAAA |
| VH7 | 282 | QVQLVQSGVEVKKP | 287 | CAGGTGCAGCTGGTCCAGAGCGGCGT |
| ADA2 | | GASVKVSCKASGYT | | GGAAGTCAAGAAACCCGGGGCCTCAG |
| | | FTNYYMYWVRQAPG | | TGAAGGTCAGCTGTAAAGCTTCCGGC |
| | | QGLEWMGGINPSNG | | TACACCTTCACAAACTACTATATGTA |
| | | GTNFNEKFKNRVTL | | TTGGGTGAGACAGGCACCAGGACAGG |
| | | TTDSSTTTAYMELK | | GACTGGAGTGGATGGGCGGGATTAAC |
| | | SLQFDDTAVYYCAR | | CCTAGTAATGGAGGCACTAACTTCAA |
| | | RDYRFDMGFDYWGQ | | CGAAAAGTTTAAAAACAGGGTGACCC |
| | | GTTVTVSSASTKGP | | TGACCACAGATTCAAGCACTACCACA |
| | | SVFPLAPCSRSTSE | | GCTTACATGGAGCTGAAGTCCCTGCA |
| | | STAALGCLVKDYFP | | GTTTGACGATACAGCCGTGTACTATT |
| | | EPVTVSWNSGALTS | | GTGCTCGGAGAGACTACAGGTTCGAT |
| | | GVHTFPAVLQSSGL | | ATGGGCTTTGACTATTGGGGCCAGGG |
| | | YSLSSVVTVPSSSL | | GACTACCGTGACCGTCTCCTCTGCTA |
| | | GTKTYTCNVDHKPS | | GCACCAAGGGCCCATCGGTCTTCCCC |
| | | NTKVDKRVESKYGP | | CTGGCGCCCTGCTCCAGGAGCACCTC |
| | | PCPPCPAPEFLGGP | | CGAGAGCACAGCCGCCCTGGGCTGCC |
| | | SVFLFPPKPKDTLM | | TGGTCAAGGACTACTTCCCCGAACCG |
| | | ISRTPEVTCVVVDV | | GTGACGGTGTCGTGGAACTCAGGCGC |
| | | SQEDPEVQFNWYVD | | CCTGACCAGCGGCGTGCACACCTTCC |
| | | GVEVHNAKTKPREE | | CGGCTGTCCTACAGTCCTCAGGACTC |
| | | QFNSTYRVVSVLTV | | TACTCCCTCAGCAGCGTGGTGACCGT |
| | | LHQDWLNGKEYKCK | | GCCCTCCAGCAGCTTGGGCACGAAGA |
| | | VSNKGLPSSIEKTI | | CCTACACCTGCAACGTAGATCACAAG |
| | | SKAKGQPREPQVYT | | CCCAGCAACACCAAGGTGGACAAGAG |
| | | LPPSQEEMTKNQVS | | AGTTGAGTCCAAATATGGTCCCCCAT |
| | | LTCLVKGFYPSDIA | | GCCCACCATGCCCAGCACCTGAGTTC |
| | | VEWESNGQPENNYK | | CTGGGGGGACCATCAGTCTTCCTGTT |
| | | TTPPVLDSDGSFFL | | CCCCCCAAAACCCAAGGACACTCTCA |
| | | YSRLTVDKSRWQEG | | TGATCTCCCGGACCCCTGAGGTCACG |
| | | NVFSCSVMHEALHN | | TGCGTGGTGGTGGACGTGAGCCAGGA |
| | | HYTQKSLSLSPGKG | | AGACCCCGAGGTCCAGTTCAACTGGT |
| | | GGGSGGGGSIDETR | | ACGTGGATGGCGTGGAGGTGCATAAT |
| | | AHLLLKEKMMRLGG | | GCCAAGACAAAGCCGCGGGAGGAGCA |
| | | RLVLNTKEELANER | | GTTCAACAGCACGTACCGTGTGGTCA |
| | | LMTLKIAEMKEAMR | | GCGTCCTCACCGTCCTGCACCAGGAC |
| | | TLIFPPSMHFFQAK | | TGGCTGAACGGCAAGGAGTACAAGTG |
| | | HLIERSQVFNILRM | | CAAGGTCTCCAACAAAGGCCTCCCGT |
| | | MPKGAALHLHDIGI | | CCTCCATCGAGAAAACCATCTCCAAA |
| | | VTMDWLVRNVTYRP | | GCCAAAGGGCAGCCCCGAGAGCCACA |
| | | HCHICFTPRGIMQF | | GGTGTACACCCTGCCCCCATCCCAGG |
| | | RFAHPTPRPSEKCS | | AGGAGATGACCAAGAACCAGGTCAGC |
| | | KWILLEDYRKRVQN | | CTGACCTGCCTGGTCAAAGGCTTCTA |
| | | VTEFDDSLLRNFTL | | CCCCAGCGACATCGCCGTGGAGTGGG |
| | | VTQHPEVIYTNQNV | | AGAGCAATGGGCAGCCGGAGAACAAC |
| | | VWSKFETIFFTISG | | TACAAGACCACGCCTCCCGTGCTGGA |
| | | LIHYAPVFRDYVFR | | CTCCGACGGCTCCTTCTTCCTCTACA |
| | | SMQEFYEDNVLYME | | GCAGGCTCACCGTGGACAAGAGCAGG |
| | | IRARLLPVYELSGE | | TGGCAGGAGGGGAATGTCTTCTCATG |
| | | HHDEEWSVKTYQEV | | CTCCGTGATGCATGAGGCTCTGCACA |
| | | AQKFVETHPEFIGI | | ACCACTACACACAGAAGAGCCTCTCC |
| | | KIIYSDHRSKDVAV | | CTGTCTCCGGGTAAAGGTGGAGGTGG |
| | | IAESIRMAMGLRIK | | TTCTGGAGGTGGAGGTAGTATCGACG |
| | | FPTVVAGFDLVGHE | | AAACCAGAGCACACTTACTGCTGAAA |
| | | DTGHSLHDYKEALM | | GAGAAAATGATGCGCCTGGGCGGGAG |
| | | IPAKDGVKLPYFFH | | ATTGGTGTTAAATACTAAGGAAGAGC |
| | | AGETDWQGTSIDRN | | TGGCAAATGAAAGACTCATGACACTG |
| | | ILDALMLNTTRIGH | | AAGATTGCTGAAATGAAGGAGGCGAT |
| | | GFALSKHPAVRTYS | | GAGGACGCTGATCTTTCCGCCTTCCA |
| | | WKKDIPIEVCPISN | | TGCACTTCTTCCAAGCTAAACACCTG |
| | | QVLKLVSDLRNHPV | | ATCGAAAGATCCCAAGTGTTTAACAT |
| | | ATLMATGHPMVISS | | CCTGAGGATGATGCCTAAGGGGGCCG |
| | | DDPAMFGAKGLSYD | | CTCTGCACCTTCACGATATTGGGATT |
| | | FYEVFMGIGGMKAD | | GTAACAATGGACTGGCTGGTAAGGAA |
| | | LRTLKQLAMNSIKY | | CGTGACATACAGACCTCATTGCCATA |
| | | STLLESEKNTFMEI | | TTTGTTTTACTCCCCGAGGAATCATG |
| | | WKKRWDKFIADVAT | | CAATTCAGGTTTGCCCACCCAACTCC |
| | | K | | TCGGCCAAGCGAGAAGTGTAGTAAGT |
| | | | | GGATTTTGCTGGAAGATTACCGTAAG |
| | | | | CGCGTGCAGAATGTGACAGAGTTTGA |
| | | | | TGACTCCCTGCTCCGCAATTTTACCC |
| | | | | TGGTGACCCAGCACCCCGAAGTTATA |
| | | | | TACACTAACCAAAATGTCGTGTGGTC |
| | | | | CAAGTTTGAGACGATCTTCTTCACGA |
| | | | | TTTCAGGCTTGATCCACTACGCCCCG |
| | | | | GTCTTTCGGGATTATGTGTTTAGGAG |
| | | | | TATGCAGGAGTTTTATGAGGATAATG |
| | | | | TTCTGTACATGGAGATCCGAGCCCGG |
| | | | | CTGCTTCCAGTCTACGAACTATCCGG |
| | | | | CGAACACCATGACGAGGAATGGAGCG |
| | | | | TCAAGACCTATCAAGAGGTGGCCCAG |
| | | | | AAGTTCGTAGAAACGCATCCAGAGTT |
| | | | | CATCGGTATTAAGATTATCTACTCTG |
| | | | | ATCACCGCTCAAAGGATGTGGCTGTC |
| | | | | ATCGCCGAGTCTATACGGATGGCCAT |
| | | | | GGGCCTGCGGATTAAGTTCCCTACCG |
| | | | | TCGTCGCCGGATTCGACCTCGTTGGG |
| | | | | CATGAGGATACTGGCCATAGTCTCCA |
| | | | | TGACTATAAAGAAGCCCTTATGATCC |
| | | | | CAGCAAAGGACGGAGTGAAGCTGCCC |
| | | | | TACTTCTTCCACGCAGGGGAGACCGA |
| | | | | CTGGCAGGGAACGAGCATCGACCGGA |
| | | | | ACATACTTGATGCACTCATGCTTAAT |
| | | | | ACCACACGAATCGGCCACGGCTTCGC |
| | | | | TCTCTCCAAGCACCCAGCCGTGAGAA |
| | | | | CCTACAGCTGGAAGAAGGATATCCCC |
| | | | | ATCGAGGTTTGTCCCATCAGCAATCA |
| | | | | GGTGCTGAAATTGGTGAGTGACCTGA |
| | | | | GAAACCACCCAGTCGCAACATTAATG |
| | | | | GCCACTGGCCACCCTATGGTGATTTC |
| | | | | AAGCGATGATCCAGCCATGTTCGGAG |
| | | | | CAAAAGGACTCAGTTACGACTTCTAT |
| | | | | GAGGTATTCATGGGTATTGGTGGTAT |
| | | | | GAAGGCAGACCTGCGGACTCTTAAGC |
| | | | | AGTTGGCAATGAACTCAATTAAGTAC |
| | | | | TCTACCTTATTGGAGTCTGAAAAGAA |
| | | | | CACATTTATGGAGATCTGGAAAAAGC |
| | | | | GCTGGGACAAATTCATCGCAGATGTT |
| | | | | GCCACAAAA |
| VH7 | 283 | QVQLVQSGVEVKKP | 288 | CAGGTGCAGCTGGTCCAGAGCGGCGT |
| ADA2 | | GASVKVSCKASGYT | | GGAAGTCAAGAAACCCGGGGCCTCAG |
| mut7 | | FTNYYMYWVRQAPG | | TGAAGGTCAGCTGTAAAGCTTCCGGC |
| | | QGLEWMGGINPSNG | | TACACCTTCACAAACTACTATATGTA |
| | | GTNFNEKFKNRVTL | | TTGGGTGAGACAGGCACCAGGACAGG |
| | | TTDSSTTTAYMELK | | GACTGGAGTGGATGGGCGGGATTAAC |
| | | SLQFDDTAVYYCAR | | CCTAGTAATGGAGGCACTAACTTCAA |
| | | RDYRFDMGFDYWGQ | | CGAAAAGTTTAAAAACAGGGTGACCC |
| | | GTTVTVSSASTKGP | | TGACCACAGATTCAAGCACTACCACA |
| | | SVFPLAPCSRSTSE | | GCTTACATGGAGCTGAAGTCCCTGCA |
| | | STAALGCLVKDYFP | | GTTTGACGATACAGCCGTGTACTATT |
| | | EPVTVSWNSGALTS | | GTGCTCGGAGAGACTACAGGTTCGAT |
| | | GVHTFPAVLQSSGL | | ATGGGCTTTGACTATTGGGGCCAGGG |
| | | YSLSSVVTVPSSSL | | GACTACCGTGACCGTCTCCTCTGCTA |
| | | GTKTYTCNVDHKPS | | GCACCAAGGGCCCATCGGTCTTCCCC |
| | | NTKVDKRVESKYGP | | CTGGCGCCCTGCTCCAGGAGCACCTC |
| | | PCPPCPAPEFLGGP | | CGAGAGCACAGCCGCCCTGGGCTGCC |
| | | SVFLFPPKPKDTLM | | TGGTCAAGGACTACTTCCCCGAACCG |
| | | ISRTPEVTCVVVDV | | GTGACGGTGTCGTGGAACTCAGGCGC |
| | | SQEDPEVQFNWYVD | | CCTGACCAGCGGCGTGCACACCTTCC |
| | | GVEVHNAKTKPREE | | CGGCTGTCCTACAGTCCTCAGGACTC |
| | | QFNSTYRVVSVLTV | | TACTCCCTCAGCAGCGTGGTGACCGT |
| | | LHQDWLNGKEYKCK | | GCCCTCCAGCAGCTTGGGCACGAAGA |
| | | VSNKGLPSSIEKTI | | CCTACACCTGCAACGTAGATCACAAG |
| | | SKAKGQPREPQVYT | | CCCAGCAACACCAAGGTGGACAAGAG |
| | | LPPSQEEMTKNQVS | | AGTTGAGTCCAAATATGGTCCCCCAT |
| | | LTCLVKGFYPSDIA | | GCCCACCATGCCCAGCACCTGAGTTC |
| | | VEWESNGQPENNYK | | CTGGGGGGACCATCAGTCTTCCTGTT |
| | | TTPPVLDSDGSFFL | | CCCCCCAAAACCCAAGGACACTCTCA |
| | | YSRLTVDKSRWQEG | | TGATCTCCCGGACCCCTGAGGTCACG |
| | | NVFSCSVMHEALHN | | TGCGTGGTGGTGGACGTGAGCCAGGA |
| | | HYTQKSLSLSPGKG | | AGACCCCGAGGTCCAGTTCAACTGGT |
| | | GGGSGGGGSIDETR | | ACGTGGATGGCGTGGAGGTGCATAAT |
| | | AHLLLKEKMMRLGG | | GCCAAGACAAAGCCGCGGGAGGAGCA |
| | | RLVLNTKEELANER | | GTTCAACAGCACGTACCGTGTGGTCA |
| | | LMTLKIAEMKEAMR | | GCGTCCTCACCGTCCTGCACCAGGAC |
| | | TLIFPPSMHFFQAK | | TGGCTGAACGGCAAGGAGTACAAGTG |
| | | HLIERSQVFNILRM | | CAAGGTCTCCAACAAAGGCCTCCCGT |
| | | MPKGAALHLHDIGI | | CCTCCATCGAGAAAACCATCTCCAAA |
| | | VTMDWLVRNVTYRP | | GCCAAAGGGCAGCCCCGAGAGCCACA |
| | | HCHICFTPRGIMQF | | GGTGTACACCCTGCCCCCATCCCAGG |
| | | RFAHPTPRPSEKCS | | AGGAGATGACCAAGAACCAGGTCAGC |
| | | KWILLEDYRKRVQN | | CTGACCTGCCTGGTCAAAGGCTTCTA |
| | | VTEFDDSLLRNFTL | | CCCCAGCGACATCGCCGTGGAGTGGG |
| | | VTQHPEVIYTNQNV | | AGAGCAATGGGCAGCCGGAGAACAAC |
| | | VWSKFETIFFTISG | | TACAAGACCACGCCTCCCGTGCTGGA |
| | | LIHYAPVFRDYVFR | | CTCCGACGGCTCCTTCTTCCTCTACA |
| | | SMQEFYEDNVLYME | | GCAGGCTCACCGTGGACAAGAGCAGG |
| | | IRAQLLPVYELSGE | | TGGCAGGAGGGGAATGTCTTCTCATG |
| | | HHDEEWSVKTYQEV | | CTCCGTGATGCATGAGGCTCTGCACA |
| | | AQKFVETHPEFIGI | | ACCACTACACACAGAAGAGCCTCTCC |
| | | KIIYNDHRSKDVAV | | CTGTCTCCGGGTAAAGGTGGAGGTGG |
| | | IAESIRMAMGLRIK | | TTCTGGAGGTGGAGGTAGTATCGACG |
| | | FPTVVAGFDLVGHE | | AAACCAGAGCACACTTACTGCTGAAA |
| | | DTGHSLHDYKEALM | | GAGAAAATGATGCGCCTGGGCGGGAG |
| | | IPAKDGVKLPYFFH | | ATTGGTGTTAAATACTAAGGAAGAGC |
| | | AGETDWQGTSIDRN | | TGGCAAATGAAAGACTCATGACACTG |
| | | ILDALMLNTTRIGH | | AAGATTGCTGAAATGAAGGAGGCGAT |
| | | GFALSKHPAVRTYS | | GAGGACGCTGATCTTTCCGCCTTCCA |
| | | WDKDIPIEVCPISN | | TGCACTTCTTCCAAGCTAAACACCTG |
| | | QVLKLVSDLRNHPV | | ATCGAAAGATCCCAAGTGTTTAACAT |
| | | ATLMATGHPMVISS | | CCTGAGGATGATGCCTAAGGGGGCCG |
| | | DDPAMFGAKGLSYD | | CTCTGCACCTTCACGATATTGGGATT |
| | | FYEVFMGIGGMKAD | | GTAACAATGGACTGGCTGGTAAGGAA |
| | | LRTLKQLAMNSIKY | | CGTGACATACAGACCTCATTGCCATA |
| | | STLLESEKNTFMEI | | TTTGTTTTACTCCCCGAGGAATCATG |
| | | WKKRWDKFIADVAT | | CAATTCAGGTTTGCCCACCCAACTCC |
| | | K | | TCGGCCAAGCGAGAAGTGTAGTAAGT |
| | | | | GGATTTTGCTGGAAGATTACCGTAAG |
| | | | | CGCGTGCAGAATGTGACAGAGTTTGA |
| | | | | TGACTCCCTGCTCCGCAATTTTACCC |
| | | | | TGGTGACCCAGCACCCCGAAGTTATA |
| | | | | TACACTAACCAAAATGTCGTGTGGTC |
| | | | | CAAGTTTGAGACGATCTTCTTCACGA |
| | | | | TTTCAGGCTTGATCCACTACGCCCCG |
| | | | | GTCTTTCGGGATTATGTGTTTAGGAG |
| | | | | TATGCAGGAGTTTTATGAGGATAATG |
| | | | | TTCTGTACATGGAGATCCGAGCCCAG |
| | | | | CTGCTTCCAGTCTACGAACTATCCGG |
| | | | | CGAACACCATGACGAGGAATGGAGCG |
| | | | | TCAAGACCTATCAAGAGGTGGCCCAG |
| | | | | AAGTTCGTAGAAACGCATCCAGAGTT |
| | | | | CATCGGTATTAAGATTATCTACAATG |
| | | | | ATCACCGCTCAAAGGATGTGGCTGTC |
| | | | | ATCGCCGAGTCTATACGGATGGCCAT |
| | | | | GGGCCTGCGGATTAAGTTCCCTACCG |
| | | | | TCGTCGCCGGATTCGACCTCGTTGGG |
| | | | | CATGAGGATACTGGCCATAGTCTCCA |
| | | | | TGACTATAAAGAAGCCCTTATGATCC |
| | | | | CAGCAAAGGACGGAGTGAAGCTGCCC |
| | | | | TACTTCTTCCACGCAGGGGAGACCGA |
| | | | | CTGGCAGGGAACGAGCATCGACCGGA |
| | | | | ACATACTTGATGCACTCATGCTTAAT |
| | | | | ACCACACGAATCGGCCACGGCTTCGC |
| | | | | TCTCTCCAAGCACCCAGCCGTGAGAA |
| | | | | CCTACAGCTGGGATAAGGATATCCCC |
| | | | | ATCGAGGTTTGTCCCATCAGCAATCA |
| | | | | GGTGCTGAAATTGGTGAGTGACCTGA |
| | | | | GAAACCACCCAGTCGCAACATTAATG |
| | | | | GCCACTGGCCACCCTATGGTGATTTC |
| | | | | AAGCGATGATCCAGCCATGTTCGGAG |
| | | | | CAAAAGGACTCAGTTACGACTTCTAT |
| | | | | GAGGTATTCATGGGTATTGGTGGTAT |
| | | | | GAAGGCAGACCTGCGGACTCTTAAGC |
| | | | | AGTTGGCAATGAACTCAATTAAGTAC |
| | | | | TCTACCTTATTGGAGTCTGAAAAGAA |
| | | | | CACATTTATGGAGATCTGGAAAAAGC |
| | | | | GCTGGGACAAATTCATCGCAGATGTT |
| | | | | GCCACAAAA |
| Human | 284 | SIDETRAHLLLKEK | | |
| ADA2 | | MMRLGGRLVLNTKE | | |
| | | ELANERLMTLKIAE | | |
| | | MKEAMRTLIFPPSM | | |
| | | HFFQAKHLIERSQV | | |
| | | FNILRMMPKGAALH | | |
| | | LHDIGIVTMDWLVR | | |
| | | NVTYRPHCHICFTP | | |
| | | RGIMQFRFAHPTPR | | |
| | | PSEKCSKWILLEDY | | |
| | | RKRVQNVTEFDDSL | | |
| | | LRNFTLVTQHPEVI | | |
| | | YTNQNVVWSKFETI | | |
| | | FFTISGLIHYAPVF | | |
| | | RDYVFRSMQEFYED | | |
| | | NVLYMEIRARLLPV | | |
| | | YELSGEHHDEEWSV | | |
| | | KTYQEVAQKFVETH | | |
| | | PEFIGIKIIYSDHR | | |
| | | SKDVAVIAESIRMA | | |
| | | MGLRIKFPTVVAGF | | |
| | | DLVGHEDTGHSLHD | | |
| | | YKEALMIPAKDGVK | | |
| | | LPYFFHAGETDWQG | | |
| | | TSIDRNILDALMLN | | |
| | | TTRIGHGFALSKHP | | |
| | | AVRTYSWKKDIPIE | | |
| | | VCPISNQVLKLVSD | | |
| | | LRNHPVATLMATGH | | |
| | | PMVISSDDPAMFGA | | |
| | | KGLSYDFYEVFMGI | | |
| | | GGMKADLRTLKQLA | | |
| | | MNSIKYSTLLESEK | | |
| | | NTFMEIWKKRWDKF | | |
| | | IADVATK | | |

Table 17. Exemplary Anti-PD-1 V_{H}/V_{L} Pairs

Table 17 provides exemplary anti-PD-1 V_{H}/V_{L} pairs for use in the fusion proteins described herein.

| Exemplary anti-PD-1 VL | Exemplary anti-PD-1 VH |
|---|---|
| Anti-PD-1 VL5 (SEQ ID NO: 12) | Anti-PD-1 VH6 (SEQ ID NO: 6) |
| Anti-PD-1 VL6 (SEQ ID NO: 13 | Anti-PD-1 VH7 (SEQ ID NO: 7) |
| Anti-PD-1 VL1 (SEQ ID NO: 8) | Anti-PD-1 VH5 (SEQ ID NO: 5) |
| Anti-PD-1 nVL1 (SEQ ID NO: 8) | Anti-PD-1 nVH3 (SEQ ID NO: 149) |
| Anti-PD-1 nVL1 (SEQ ID NO: 8) | Anti-PD-1 nVH7 (SEQ ID NO: 157) |
| Anti PD-1 nVL1 (SEQ ID NO: 8) | Anti-PD-1 nVH8 (SEQ ID NO: 158) |

### Table 18. Exemplary Anti-PD-1-TGFbRII ECD Fusion Protein Sequences

Table 18 provides exemplary anti-PD-1-TGFbRII ECD fusion protein sequences. The anti-PD-1 fusion proteins comprise a sequence encoding an anti-PD-1 variable region of light chain and a sequence encoding an anti-PD-1 variable region of heavy chain. ECD stands for extracellular domain. As used herein, the abbreviation “wt” refers to the wild type sequence and the abbreviation “mut” refers to a mutant sequence.

| Exemplary VL | Exemplary VH-IgG4-ECD |
|---|---|
| VL5 (SEQ ID NO: 15) | VH6-IgG4(wt)-linker-ECD (SEQ NO: 16) |
| VL5 (SEQ ID NO: 15) | VH6-IgG4(mut)-linker-ECD (SEQ NO: 143) |
| VL5 (SEQ ID NO: 15) | VH6-IgG4(mut)-linker-ECD (SEQ NO: 294) |
| VL6 (SEQ ID NO: 296) | VH7-IgG4(wt)-linker-ECD (SEQ NO: 145) |
| VL6 (SEQ ID NO: 296) | VH7-IgG4(mut)-linker-ECD (SEQ NO: 144) |
| VL6 (SEQ ID NO: 296) | VH7-IgG4(mut)-linker-ECD (SEQ NO: 295) |

### Table 19. Exemplary Anti-PD-1-ADA2 Fusion Protein Sequences

Table 19 provides exemplary anti-PD-1-ADA2 fusion protein sequences. The anti-PD-1 fusion proteins comprise a sequence encoding an anti-PD-1 variable region of light chain and a sequence encoding an anti-PD-1 variable region of heavy chain.

| Exemplary VL | Exemplary VH-IgG4-ADA2 |
|---|---|
| VL5 (SEQ ID NO: 12) | VH6-IgG4(mut)-linker-ADA2 (wt) |
| | (SEQ NO. 280) |
| VL5 (SEQ ID NO: 12) | VH6-IgG4(mut)-linker-ADA2 (mut 7) |
| | (SEQ NO. 281) |
| VL6 (SEQ ID NO: 13) | VH7-IgG4(mut)-linker-ADA2 (wt) |
| | (SEQ NO. 282) |
| VL6 (SEQ ID NO: 13) | VH7-IgG4(mut)-linker-ADA2 (mut 7) |
| | (SEQ NO. 283) |

### Table 20. Exemplary Anti-PD-1 Heavy Chain CDR Sequences

Table 20 provides exemplary CDR sequences for use in anti-PD-1 heavy chains.

| | SEQ ID | | SEQ ID | |
|---|---|---|---|---|
| Name | NO | Amino acid sequence | NO | Nucleotide Sequence |
| Anti- | 310 | Ser Thr Thr Tyr Tyr | 344 | AGTACTACTTACTACTGGGTC |
| PD-1 | | Trp Val | | |
| heavy | | | | |
| chain | | | | |
| CDR | | | | |
| Anti- | 311 | Ser Ile Ser Tyr Ser | 345 | AGTATCTCTTATAGTGGGAAC |
| PD-1 | | Gly Asn Thr Tyr Tyr | | ACCTACTACAATCCGTCCCTC |
| heavy | | Asn Pro Ser Leu Lys | | AAGAGT |
| chain | | Ser | | |
| CDR | | | | |
| Anti- | 312 | His Leu Gly Tyr Asn | 346 | CATCTAGGGTATAATGGGAGG |
| PD-1 | | Gly Arg Tyr Leu Pro | | TACCTCCCCTTTGACTAC |
| heavy | | Phe Asp Tyr | | |
| chain | | | | |
| CDR | | | | |
| Anti- | 313 | Ser Ser Thr Tyr Tyr | 347 | AGTAGTACTTACTACTGGGGC |
| PD-1 | | Trp Gly | | |
| heavy | | | | |
| chain | | | | |
| CDR | | | | |
| Anti- | 314 | Ser Ile Ser Tyr Ser | 348 | AGTATCTCTTATAGTGGGAGC |
| PD-1 | | Gly Ser Thr Tyr Tyr | | ACCTACTACAATCCGTCCCTC |
| heavy | | Asn Pro Ser Leu Lys | | AAGAGT |
| chain | | Ser | | |
| CDR | | | | |
| Anti- | 315 | Ser Thr Thr Tyr Tyr | 349 | AGTACTACTTACTACTGGGGC |
| PD-1 | | Trp Gly | | |
| heavy | | | | |
| chain | | | | |
| CDR | | | | |
| Anti- | 316 | Ser Ile Ser Tyr Ser | 350 | AGTATCTCTTATAGTGGGACC |
| PD-1 | | Gly Thr Thr Tyr Tyr | | ACCTACTACAACCCGTCCCTC |
| heavy | | Asn Pro Ser Leu Lys | | AAGAGT |
| chain | | Ser | | |
| CDR | | | | |
| Anti- | 317 | His Leu Gly Tyr Asn | 351 | CATCTCGGGTATAACAGCAAC |
| PD-1 | | Ser Asn Trp Tyr Pro | | TGGTACCCTTTTGACTAC |
| heavy | | Phe Asp Tyr | | |
| chain | | | | |
| CDR | | | | |
| Anti- | 318 | Ser His Ala Met Ser | 352 | AGCCATGCCATGAGC |
| PD-1 | | | | |
| heavy | | | | |
| chain | | | | |
| CDR | | | | |
| Anti- | 319 | Thr Ile Thr Gly Gly | 353 | ACTATTACTGGTGGTGGTGGT |
| PD-1 | | Gly Gly Ser Ile Tyr | | AGCATATACTACGCAGACTCC |
| heavy | | Tyr Ala Asp Ser Val | | GTGAAGGGC |
| chain | | Lys | | |
| CDR | | | | |
| Anti- | 320 | Asn Arg Ala Gly Glu | 354 | AACCGCGCTGGGGAGGGTTAC |
| PD-1 | | Gly Tyr Phe Asp Tyr | | TTTGACTAC |
| heavy | | | | |
| chain | | | | |
| CDR | | | | |
| Anti- | 378 | Ser Tyr Ala Ile Ser | | |
| PD-1 | | | | |
| heavy | | | | |
| chain | | | | |
| CDR | | | | |
| Anti- | 379 | Gly Ile Ile Pro Ile | | |
| PD-1 | | Phe Asp Thr Ala Asn | | |
| heavy | | Tyr Ala Gln Lys Phe | | |
| chain | | Gln | | |
| CDR | | | | |
| Anti- | 380 | Pro Gly Leu Ala Ala | | |
| PD-1 | | Ala Tyr Asp Thr Gly | | |
| heavy | | Ser Leu Asp Tyr | | |
| chain | | | | |
| CDR | | | | |

### Table 21. Exemplary Anti-PD-1 Light Chain CDR Sequences

Table 21 provides exemplary CDR sequences for use in anti-PD-1 light chains.

| | SEQ ID | | SEQ ID | |
|---|---|---|---|---|
| Name | NO | Amino acid sequence | NO | Nucleotide Sequence |
| Anti- | 321 | Thr Gly Thr Ser Ser | 355 | ACTGGAACCAGCAGTGACGTT |
| PD-1 | | Asp Val Gly Phe Tyr | | GGTTTTTATAACTATGTCTCC |
| light | | Asn Tyr Val Ser | | |
| chain | | | | |
| CDR | | | | |
| Anti- | 322 | Asp Val Thr Asn Arg | 356 | GATGTCACTAATCGGCCCTCA |
| PD-1 | | Pro Ser | | |
| light | | | | |
| chain | | | | |
| CDR | | | | |
| Anti- | 323 | Ser Ser Tyr Thr Ser | 357 | AGCTCATATACAAGCATCAGC |
| PD-1 | | Ile Ser Thr Trp Val | | ACTTGGGTG |
| light | | | | |
| chain | | | | |
| CDR | | | | |
| Anti- | 324 | Asp Val Ser Asn Arg | 358 | GATGTCAGTAATCGGCCCTCA |
| PD-1 | | Pro Ser | | |
| light | | | | |
| chain | | | | |
| CDR | | | | |
| Anti- | 325 | Ser Ser Tyr Thr Asn | 359 | AGCTCATATACAAACATCAGC |
| PD-1 | | Ile Ser Thr Trp Val | | ACTTGGGTG |
| light | | | | |
| chain | | | | |
| CDR | | | | |
| Anti- | 326 | Thr Gly Thr Ser Ser | 360 | ACTGGAACCAGCAGTGACGTT |
| PD-1 | | Asp Val Gly Ser Tyr | | GGTAGTTATAACCGTGTCTCC |
| light | | Asn Arg Val Ser | | |
| chain | | | | |
| CDR | | | | |
| Anti- | 327 | Glu Val Ser Asn Arg | 361 | GAGGTCAGTAATCGGCCCTCA |
| PD-1 | | Pro Ser | | |
| light | | | | |
| chain | | | | |
| CDR | | | | |
| Anti- | 328 | Ser Ser Tyr Thr Ser | 362 | AGCTCATATACAAGCAGCAGC |
| PD-1 | | Ser Ser Thr Trp Val | | ACTTGGGTG |
| light | | | | |
| chain | | | | |
| CDR | | | | |
| Anti- | 329 | Gly Gly Asp Asn Ile | 363 | GGGGGAGACAACATTGGAAAT |
| PD-1 | | Gly Asn Lys Asp Val | | AAAGATGTGCAC |
| light | | His | | |
| chain | | | | |
| CDR | | | | |
| Anti- | 330 | Arg Asp Ser Asn Arg | 364 | AGGGATAGCAACCGGCCCTCT |
| PD-1 | | Pro Ser | | |
| light | | | | |
| chain | | | | |
| CDR | | | | |
| Anti- | 331 | Gln Val Trp Asp Ser | 365 | CAGGTGTGGGACAGCATTTGG |
| PD-1 | | Ile Trp Val | | GTG |
| light | | | | |
| chain | | | | |
| CDR | | | | |
| Anti- | 332 | Ser Ser Tyr Thr Ser | 366 | AGCTCATATACAAGCATCAGC |
| PD-1 | | Ile Ser Thr Trp Val | | ACTTGGGTG |
| light | | | | |
| chain | | | | |
| CDR | | | | |
| Anti- | 381 | Arg Ala Ser Gln Ser | | |
| PD-1 | | Val Arg Ser Tyr Leu | | |
| light | | Ala | | |
| chain | | | | |
| CDR | | | | |
| Anti- | 382 | Asp Ala Ser Asn Arg | | |
| PD-1 | | Ala Thr | | |
| light | | | | |
| chain | | | | |
| CDR | | | | |
| Anti- | 383 | Gln Gln Arg Asn Tyr | | |
| PD-1 | | Trp Pro Leu Thr | | |
| light | | | | |
| chain | | | | |
| CDR | | | | |

### Table 22. Exemplary Anti-PD-1 Heavy Chain Variable Region Sequences

Table 22 provides exemplary sequences for use in anti-PD-1 heavy chain variable regions.

| | SEQ | | SEQ | |
|---|---|---|---|---|
| | ID | | ID | |
| Name | NO | Amino acid sequence | NO | Nucleotide Sequence |
| Anti- | 333 | Gln Leu Gln Leu Gln | 367 | CAGCTGCAGCTGCAGGAGTCGGGCCC |
| PD-1 | | Glu Ser Gly Pro Gly | | AGGACTGGTGAAGCCTTCGGAGACCC |
| heavy | | Leu Val Lys Pro Ser | | TGACCCTCACCTGCACTGTCTCTGGT |
| chain | | Glu Thr Leu Thr Leu | | GACTCCATCAGCAGTACTACTTACTA |
| variable | | Thr Cys Thr Val Ser | | CTGGGTCTGGATCCGCCAGCCCCCAG |
| region | | Gly Asp Ser Ile Ser | | GGAAGGGACTGGAGTGGATTGGGAGT |
| | | Ser Thr Thr Tyr Tyr | | ATCTCTTATAGTGGGAACACCTACTA |
| | | Trp Val Trp Ile Arg | | CAATCCGTCCCTCAAGAGTCGAGTCA |
| | | Gln Pro Pro Gly Lys | | CCATATCCGTAGACACGTCCAAGAAC |
| | | Gly Leu Glu Trp Ile | | CACTTCTCCCTGAAGCTGAGTTCTGT |
| | | Gly Ser Ile Ser Tyr | | GGCCGCCACAGACACGGCTCTATATT |
| | | Ser Gly Asn Thr Tyr | | ACTGTGCGAGACATCTAGGGTATAAT |
| | | Tyr Asn Pro Ser Leu | | GGGAGGTACCTCCCCTTTGACTACTG |
| | | Lys Ser Arg Val Thr | | GGGCCAGGGAACCCTGGTCACCGTCT |
| | | Ile Ser Val Asp Thr | | CCTCC |
| | | Ser Lys Asn His Phe | | |
| | | Ser Leu Lys Leu Ser | | |
| | | Ser Val Ala Ala Thr | | |
| | | Asp Thr Ala Leu Tyr | | |
| | | Tyr Cys Ala Arg His | | |
| | | Leu Gly Tyr Asn Gly | | |
| | | Arg Tyr Leu Pro Phe | | |
| | | Asp Tyr Trp Gly Gln | | |
| | | Gly Thr Leu Val Thr | | |
| | | Val Ser Ser | | |
| Anti- | 334 | Gln Leu Gln Leu Gln | 368 | CAGCTGCAGCTGCAGGAGTCGGGCCC |
| PD-1 | | Glu Ser Gly Pro Gly | | AGGACTGGTGAAGCCTTCGGAGACCC |
| heavy | | Leu Val Lys Pro Ser | | TGTCCCTCACCTGCACTGTCTCTGGT |
| chain | | Glu Thr Leu Ser Leu | | GGCTCCATCAGCAGTAGTACTTACTA |
| variable | | Thr Cys Thr Val Ser | | CTGGGGCTGGATCCGCCAGCCCCCAG |
| region | | Gly Gly Ser Ile Ser | | GGAAGGGACTGGAGTGGATTGGGAGT |
| | | Ser Ser Thr Tyr Tyr | | ATCTCTTATAGTGGGAGCACCTACTA |
| | | Trp Gly Trp Ile Arg | | CAATCCGTCCCTCAAGAGTCGAGTCA |
| | | Gln Pro Pro Gly Lys | | CCATATCCGTAGACACGTCCAAGAAC |
| | | Gly Leu Glu Trp Ile | | CAGTTCTCCCTGAAGCTGAGCTCTGT |
| | | Gly Ser Ile Ser Tyr | | GACCGACGCAGACACGGCTGTGTATT |
| | | Ser Gly Ser Thr Tyr | | ACTGTGCGAGACATCTAGGGTATAAT |
| | | Tyr Asn Pro Ser Leu | | GGGAGGTACCTCCCCTTTGACTACTG |
| | | Lys Ser Arg Val Thr | | GGGCCAGGGAACCCTGGTCACCGTCT |
| | | Ile Ser Val Asp Thr | | CCTCC |
| | | Ser Lys Asn Gln Phe | | |
| | | Ser Leu Lys Leu Ser | | |
| | | Ser Val Thr Asp Ala | | |
| | | Asp Thr Ala Val Tyr | | |
| | | Tyr Cys Ala Arg His | | |
| | | Leu Gly Tyr Asn Gly | | |
| | | Arg Tyr Leu Pro Phe | | |
| | | Asp Tyr Trp Gly Gln | | |
| | | Gly Thr Leu Val Thr | | |
| | | Val Ser Ser | | |
| Anti- | 335 | Gln Leu Gln Leu Gln | 369 | CAGCTGCAGCTGCAGGAGTCGGGCCC |
| PD-1 | | Glu Ser Gly Pro Gly | | AGGACTGGTGAAGCCTTCGGAGACCC |
| heavy | | Leu Val Lys Pro Ser | | TGACCCTCACCTGCACTGTCTCTGCT |
| chain | | Glu Thr Leu Thr Leu | | GACTCCATCAGCAGTACTACTTACTA |
| variable | | Thr Cys Thr Val Ser | | CTGGGTCTGGATCCGCCAGCCCCCAG |
| region | | Ala Asp Ser Ile Ser | | GGAAGGGACTGGAGTGGATTGGGAGT |
| | | Ser Thr Thr Tyr Tyr | | ATCTCTTATAGTGGGAGCACCTACTA |
| | | Trp Val Trp Ile Arg | | CAATCCGTCCCTCAAGAGTCGAGTCA |
| | | Gln Pro Pro Gly Lys | | CCGTATCCGTAGACACGTCCAAGAAC |
| | | Gly Leu Glu Trp Ile | | CAGTTCTCCCTGAAGCTGAACTCTGT |
| | | Gly Ser Ile Ser Tyr | | GGCCGCCACAGACACGGCTCTATATT |
| | | Ser Gly Ser Thr Tyr | | ACTGTGCGAGACATCTAGGGTATAAT |
| | | Tyr Asn Pro Ser Leu | | GGGAGGTACCTCCCCTTTGACTACTG |
| | | Lys Ser Arg Val Thr | | GGGCCAGGGAACCCTGGTCACCGTCT |
| | | Val Ser Val Asp Thr | | CCTCC |
| | | Ser Lys Asn Gln Phe | | |
| | | Ser Leu Lys Leu Asn | | |
| | | Ser Val Ala Ala Thr | | |
| | | Asp Thr Ala Leu Tyr | | |
| | | Tyr Cys Ala Arg His | | |
| | | Leu Gly Tyr Asn Gly | | |
| | | Arg Tyr Leu Pro Phe | | |
| | | Asp Tyr Trp Gly Gln | | |
| | | Gly Thr Leu Val Thr | | |
| | | Val Ser Ser | | |
| Anti- | 336 | Gln Leu Gln Leu Gln | 370 | CAGCTGCAGCTGCAGGAGTCGGGCCC |
| PD-1 | | Glu Ser Gly Pro Gly | | AGGACTGGTGAAGCCCTCGGAGACCC |
| heavy | | Leu Val Lys Pro Ser | | TGTCCCTCACCTGCACTGTCTCTGGT |
| chain | | Glu Thr Leu Ser Leu | | GGCTCCATCAGCAGTACTACTTACTA |
| variable | | Thr Cys Thr Val Ser | | CTGGGGCTGGATCCGCCAGCCCCCAG |
| region | | Gly Gly Ser Ile Ser | | GGAAGGGGCTGGAGTGGATTGGGAGT |
| | | Ser Thr Thr Tyr Tyr | | ATCTCTTATAGTGGGACCACCTACTA |
| | | Trp Gly Trp Ile Arg | | CAACCCGTCCCTCAAGAGTCGAGTCA |
| | | Gln Pro Pro Gly Lys | | CCATCCCCGTAGACACGTCCAAGAAC |
| | | Gly Leu Glu Trp Ile | | CAGATCTCCCTGAAACTGAGCTCTGT |
| | | Gly Ser Ile Ser Tyr | | GACCGCCGCAGACACGTCTTTGTATT |
| | | Ser Gly Thr Thr Tyr | | ATTGTGCGAGACATCTCGGGTATAAC |
| | | Tyr Asn Pro Ser Leu | | AGCAACTGGTACCCTTTTGACTACTG |
| | | Lys Ser Arg Val Thr | | GGGCCAGGGAACCCTGGTCACCGTCT |
| | | Ile Pro Val Asp Thr | | CCTCA |
| | | Ser Lys Asn Gln Ile | | |
| | | Ser Leu Lys Leu Ser | | |
| | | Ser Val Thr Ala Ala | | |
| | | Asp Thr Ser Leu Tyr | | |
| | | Tyr Cys Ala Arg His | | |
| | | Leu Gly Tyr Asn Ser | | |
| | | Asn Trp Tyr Pro Phe | | |
| | | Asp Tyr Trp Gly Gln | | |
| | | Gly Thr Leu Val Thr | | |
| | | Val Ser Ser | | |
| Anti- | 337 | Glu Val Gln Leu Leu | 371 | GAGGTGCAGCTGTTGGAGTCTGGGGG |
| PD-1 | | Glu Ser Gly Gly Gly | | AGGCTTGGTACAGCCTGGGGGGTCCC |
| heavy | | Leu Val Gln Pro Gly | | TGAGACTGTCCTGCGCAGCCTCTGGA |
| chain | | Gly Ser Leu Arg Leu | | TTCACCTTTAGCAGCCATGCCATGAG |
| variable | | Ser Cys Ala Ala Ser | | CTGGGTCCGCCAGGCTCCAGGGAAGG |
| region | | Gly Phe Thr Phe Ser | | GGCTGGAGTGGGTCTCAACTATTACT |
| | | Ser His Ala Met Ser | | GGTGGTGGTGGTAGCATATACTACGC |
| | | Trp Val Arg Gln Ala | | AGACTCCGTGAAGGGCCGGTTCACCA |
| | | Pro Gly Lys Gly Leu | | TCTCCAGAGACAATTCCAAGAACACG |
| | | Glu Trp Val Ser Thr | | CTGTATCTGCAAATGAACAGCCTGAG |
| | | Ile Thr Gly Gly Gly | | AGCCGAGGACACGGCCGTATATTATT |
| | | Gly Ser Ile Tyr Tyr | | GTGCGAAAAACCGCGCTGGGGAGGGT |
| | | Ala Asp Ser Val Lys | | TACTTTGACTACTGGGGCCAGGGAAC |
| | | Gly Arg Phe Thr Ile | | CCTGGTCACCGTCTCCTCA |
| | | Ser Arg Asp Asn Ser | | |
| | | Lys Asn Thr Leu Tyr | | |
| | | Leu Gln Met Asn Ser | | |
| | | Leu Arg Ala Glu Asp | | |
| | | Thr Ala Val Tyr Tyr | | |
| | | Cys Ala Lys Asn Arg | | |
| | | Ala Gly Glu Gly Tyr | | |
| | | Phe Asp Tyr Trp Gly | | |
| | | Gln Gly Thr Leu Val | | |
| | | Thr Val Ser Ser | | |
| Anti- | 384 | Gln Val Gln Leu Val | | |
| PD-1 | | Gln Ser Gly Ala Glu | | |
| heavy | | Val Lys Lys Pro Gly | | |
| chain | | Ser Ser Val Lys Val | | |
| variable | | Ser Cys Lys Ala Ser | | |
| region | | Gly Gly Thr Phe Ser | | |
| | | Ser Tyr Ala Ile Ser | | |
| | | Trp Val Arg Gln Ala | | |
| | | Pro Gly Gln Gly Leu | | |
| | | Glu Trp Met Gly Gly | | |
| | | Ile Ile Pro Ile Phe | | |
| | | Asp Thr Ala Asn Tyr | | |
| | | Ala Gln Lys Phe Gln | | |
| | | Gly Arg Val Thr Ile | | |
| | | Thr Ala Asp Glu Ser | | |
| | | Thr Ser Thr Ala Tyr | | |
| | | Met Glu Leu Ser Ser | | |
| | | Leu Arg Ser Glu Asp | | |
| | | Thr Ala Val Tyr Tyr | | |
| | | Cys Ala Arg Pro Gly | | |
| | | Leu Ala Ala Ala Tyr | | |
| | | Asp Thr Gly Ser Leu | | |
| | | Asp Tyr Trp Gly Gln | | |
| | | Gly Thr Leu Val Thr | | |
| | | Val Ser Ser | | |

### Table 23. Exemplary Anti-PD-1 Light Chain Variable Region Sequences

Table 23 provides exemplary sequences for use in anti-PD-1 light chain variable regions.

| | SEQ | | SEQ | |
|---|---|---|---|---|
| | ID | | ID | |
| Name | NO | Amino acid sequence | NO | Nucleotide sequence |
| Anti- | 338 | Gln Ser Ala Leu Thr | 372 | CAGTCTGCCCTGACTCAGCCTGCCTCC |
| PD-1 | | Gln Pro Ala Ser Val | | GTGTCTGGGTCTCCTGGACAGTCGATC |
| light | | Ser Gly Ser Pro Gly | | ACCATCTCCTGCACTGGAACCAGCAGT |
| chain | | Gln Ser Ile Thr Ile Ser | | GACGTTGGTTTTTATAACTATGTCTCCT |
| variable | | Cys Thr Gly Thr Ser | | GGTACCAACAGCACCCAGGCAAAGCC |
| region | | Ser Asp Val Gly Phe | | CCCGAACTCATGATTTATGATGTCACT |
| | | Tyr Asn Tyr Val Ser | | AATCGGCCCTCAGGGGTTTCTGATCGC |
| | | Trp Tyr Gln Gln His | | TTCTCTGGCTCCAAGTCTGGCAACACG |
| | | Pro Gly Lys Ala Pro | | GCCTCCCTGACCATCTCTGGGCTCCAG |
| | | Glu Leu Met Ile Tyr | | GCTGAGGACGAGGCTGATTATTACTGC |
| | | Asp Val Thr Asn Arg | | AGCTCATATACAAGCATCAGCACTTGG |
| | | Pro Ser Gly Val Ser | | GTGTTCGGCGGAGGGACCAAGCTGAC |
| | | Asp Arg Phe Ser Gly | | CGTCCTA |
| | | Ser Lys Ser Gly Asn | | |
| | | Thr Ala Ser Leu Thr | | |
| | | Ile Ser Gly Leu Gln | | |
| | | Ala Glu Asp Glu Ala | | |
| | | Asp Tyr Tyr Cys Ser | | |
| | | Ser Tyr Thr Ser Ile Ser | | |
| | | Thr Trp Val Phe Gly | | |
| | | Gly Gly Thr Lys Leu | | |
| | | Thr Val Leu | | |
| Anti- | 339 | Gln Ser Ala Leu Thr | 373 | CAGTCTGCCCTGACTCAGCCTGCCTCC |
| PD-1 | | Gln Pro Ala Ser Val | | GTGTCTGGGTCTCCTGGACAGTCGATC |
| light | | Ser Gly Ser Pro Gly | | ACCATCTCCTGCACTGGAACCAGCAGT |
| chain | | Gln Ser Ile Thr Ile Ser | | GACGTTGGTTTTTATAACTATGTCTCCT |
| variable | | Cys Thr Gly Thr Ser | | GGTACCAACAGCACCCAGGCAAAGCC |
| region | | Ser Asp Val Gly Phe | | CCCGAAGTCATGATTTATGATGTCAGT |
| | | Tyr Asn Tyr Val Ser | | AATCGGCCCTCAGGGGTTTCTGATCGC |
| | | Trp Tyr Gln Gln His | | TTCTCTGGCTCCAAGTCTGGCAACACG |
| | | Pro Gly Lys Ala Pro | | GCCTCCCTGACTATCTCTGGGCTCCAG |
| | | Glu Val Met Ile Tyr | | GCTGAGGACGAGGCTGATTATTACTGC |
| | | Asp Val Ser Asn Arg | | AGCTCATATACAAGCATCAGCACTTGG |
| | | Pro Ser Gly Val Ser | | GTGTTCGGCGGAGGGACCAAGCTGAC |
| | | Asp Arg Phe Ser Gly | | TGTCCTA |
| | | Ser Lys Ser Gly Asn | | |
| | | Thr Ala Ser Leu Thr | | |
| | | Ile Ser Gly Leu Gln | | |
| | | Ala Glu Asp Glu Ala | | |
| | | Asp Tyr Tyr Cys Ser | | |
| | | Ser Tyr Thr Ser Ile Ser | | |
| | | Thr Trp Val Phe Gly | | |
| | | Gly Gly Thr Lys Leu | | |
| | | Thr Val Leu | | |
| Anti- | 340 | Gln Ser Ala Leu Thr | 374 | CAGTCTGCCCTGACTCAGCCTGCCTCC |
| PD-1 | | Gln Pro Ala Ser Val | | GTGTCTGGGTCTCCTGGACAGTCGATC |
| light | | Ser Gly Ser Pro Gly | | ACCATCTCCTGCACTGGAACCAGCAGT |
| chain | | Gln Ser Ile Thr Ile Ser | | GACGTTGGTTTTTATAACTATGTCTCCT |
| variable | | Cys Thr Gly Thr Ser | | GGTACCAACAGCACCCAGGCAAAGCC |
| region | | Ser Asp Val Gly Phe | | CCCGAACTCATGATTTATGATGTCAGT |
| | | Tyr Asn Tyr Val Ser | | AATCGGCCCTCAGGGGTTTCTGATCGC |
| | | Trp Tyr Gln Gln His | | TTCTCTGGCTCCAAGTCTGGCAACACG |
| | | Pro Gly Lys Ala Pro | | GCCTCCCTGACCATCTCTGGGCTCCAG |
| | | Glu Leu Met Ile Tyr | | GCTGAGGACGAGGCTGATTATTACTGC |
| | | Asp Val Ser Asn Arg | | AGCTCATATACAAACATCAGCACTTGG |
| | | Pro Ser Gly Val Ser | | GTGTTCGGCGGAGGGACCAAGCTGAC |
| | | Asp Arg Phe Ser Gly | | CGTCCTA |
| | | Ser Lys Ser Gly Asn | | |
| | | Thr Ala Ser Leu Thr | | |
| | | Ile Ser Gly Leu Gln | | |
| | | Ala Glu Asp Glu Ala | | |
| | | Asp Tyr Tyr Cys Ser | | |
| | | Ser Tyr Thr Asn Ile Ser | | |
| | | Thr Trp Val Phe Gly | | |
| | | Gly Gly Thr Lys Leu | | |
| | | Thr Val Leu | | |
| Anti- | 341 | Gln Ser Ala Leu Thr | 375 | CAGTCGGCCCTGACTCAGCCTCCCTCC |
| PD-1 | | Gln Pro Pro Ser Val | | GTGTCCGGGTCTCCTGGACAGTCAGTC |
| light | | Ser Gly Ser Pro Gly | | ACCATCTCCTGCACTGGAACCAGCAGT |
| chain | | Gln Ser Val Thr Ile Ser | | GACGTTGGTAGTTATAACCGTGTCTCC |
| variable | | Cys Thr Gly Thr Ser | | TGGTACCAGCAGCCCCCAGGCACAGC |
| region | | Ser Asp Val Gly Ser | | CCCCGAAGTCATTATTTATGAGGTCAG |
| | | Tyr Asn Arg Val Ser | | TAATCGGCCCTCAGGGGTCCCTGATCG |
| | | Trp Tyr Gln Gln Pro | | CTTCTCTGGGTCCAAGTCTGGCAACAC |
| | | Pro Gly Thr Ala Pro | | GGCCTCCCTGACCATCTCTGGGCTCCA |
| | | Glu Val Ile Ile Tyr Glu | | GGCTGAGGACGAGGCTGATTATTACTG |
| | | Val Ser Asn Arg Pro | | CAGCTCATATACAAGCAGCAGCACTTG |
| | | Ser Gly Val Pro Asp | | GGTGTTCGGCGGAGGGACCAAGCTGA |
| | | Arg Phe Ser ly Ser Lys | | CCGTCCTA |
| | | Ser Gly Asn Thr Ala | | |
| | | Ser Leu Thr Ile Ser Gly | | |
| | | Leu Gln Ala Glu Asp | | |
| | | Glu Ala Asp Tyr Tyr | | |
| | | Cys Ser Ser Tyr Thr | | |
| | | Ser Ser Ser Thr Trp | | |
| | | Val Phe Gly Gly Gly | | |
| | | Thr Lys Leu Thr Val | | |
| | | Leu | | |
| Anti- | 342 | Ser Tyr Glu Leu Thr | 376 | TCCTATGAGCTGACTCAGCCACTCTCA |
| PD-1 | | Gln Pro Leu Ser Val | | GTGTCAGTGGCCCTGGGACAGACGGC |
| light | | Ser Val Ala Leu Gly | | CAGGATTACCTGTGGGGGAGACAACA |
| chain | | Gln Thr Ala Arg Ile | | TTGGAAATAAAGATGTGCACTGGTACC |
| variable | | Thr Cys Gly Gly Asp | | AGCAGAAGCCAGGCCAGGCCCCTGTG |
| region | | Asn Ile Gly Asn Lys | | CTGGTCATCTATAGGGATAGCAACCGG |
| | | Asp Val His Trp Tyr | | CCCTCTGGGATCCCTGAGGGATTCTCT |
| | | Gln Gln Lys Pro Gly | | GGCTCCAACTCGGGGAACACGGCCAC |
| | | Gln Ala Pro Val Leu | | CCTGACCATCAGCAGAGCCCAAGCCG |
| | | Val Ile Tyr Arg Asp | | GGGATGAGGCTGACTATTACTGTCAGG |
| | | Ser Asn Arg Pro Ser | | TGTGGGACAGCATTTGGGTGTTCGGCG |
| | | Gly Ile Pro Glu Gly | | GAGGGACCAAGCTGACCGTCCTA |
| | | Phe Ser Gly Ser Asn | | |
| | | Ser Gly Asn Thr Ala | | |
| | | Thr Leu Thr Ile Ser | | |
| | | Arg Ala Gln Ala Gly | | |
| | | Asp Glu Ala Asp Tyr | | |
| | | Tyr Cys Gln Val Trp | | |
| | | Asp Ser Ile Trp Val | | |
| | | Phe Gly Gly Gly Thr | | |
| | | Lys Leu Thr Val Leu | | |
| Anti- | 343 | Gln Ser Ala Leu Thr | 377 | CAGTCTGCCCTGACTCAGCCTGCCTCC |
| PD-1 | | Gln Pro Ala Ser Val | | GTGTCTGGGTCTCCTGGACAGTCGATC |
| light | | Ser Gly Ser Pro Gly | | ACCATCTCCTGCACTGGAACCAGCAGT |
| chain | | Gln Ser Ile Thr Ile Ser | | GACGTTGGTTTTTATAACTATGTCTCCT |
| variable | | Cys Thr Gly Thr Ser | | GGTACCAACAGCACCCAGGCAAAGCC |
| region | | Ser Asp Val Gly Phe | | CCCGAACTCATGATTTATGATGTCAGT |
| | | Tyr Asn Tyr Val Ser | | AATCGGCCCTCAGGGGTTTCTGATCGC |
| | | Trp Tyr Gln Gln His | | TTCTCTGGCTCCAAGTCTGGCAACACG |
| | | Pro Gly Lys Ala Pro | | GCCTCCCTGACCATCTCTGGGCTCCAG |
| | | Glu Leu Met Ile Tyr | | GCTGAGGACGAGGCTGATTATTACTGC |
| | | Asp Val Ser Asn Arg | | AGCTCATATACAAGCATCAGCACTTGG |
| | | Pro Ser Gly Val Ser | | GTGTTCGGCGGAGGGACCAAGCTGAC |
| | | Asp Arg Phe Ser Gly | | CGTCCTA |
| | | Ser Lys Ser Gly Asn | | |
| | | Thr Ala Ser Leu Thr | | |
| | | Ile Ser Gly Leu Gln | | |
| | | Ala Glu Asp Glu Ala | | |
| | | Asp Tyr Tyr Cys Ser | | |
| | | Ser Tyr Thr Ser Ile Ser | | |
| | | Thr Trp Val Phe Gly | | |
| | | Gly Gly Thr Lys Leu | | |
| | | Thr Val Leu | | |
| Anti- | 385 | Glu Ile Val Leu Thr | | |
| PD-1 | | Gln Ser Pro Ala Thr | | |
| light | | Leu Ser Leu Ser Pro | | |
| chain | | Gly Glu Arg Ala Thr | | |
| variable | | Leu Ser Cys Arg Ala | | |
| region | | Ser Gln Ser Val Arg | | |
| | | Ser Tyr Leu Ala Trp | | |
| | | Tyr Gln Gln Lys Pro | | |
| | | Gly Gln Ala Pro Arg | | |
| | | Leu Leu Ile Tyr Asp | | |
| | | Ala Ser Asn Arg Ala | | |
| | | Thr Gly Ile Pro Ala | | |
| | | Arg Phe Ser Gly Ser | | |
| | | Gly Ser Gly Thr Asp | | |
| | | Phe Thr Leu Thr Ile | | |
| | | Ser Ser Leu Glu Pro | | |
| | | Glu Asp Phe Ala Val | | |
| | | Tyr Tyr Cys Gln Gln | | |
| | | Arg Asn Tyr Trp Pro | | |
| | | Leu Thr Phe Gly Gln | | |
| | | Gly Thr Lys Val Glu | | |
| | | Ile Lys | | |

### Table 24. Exemplary Anti-PD-1 Heavy Chain Sequence

Table 24 provides an exemplary sequence for use in an anti-PD-1 heavy chain.

| | SEQ | | SEQ | |
|---|---|---|---|---|
| | ID | | ID | |
| Name | NO | Amino acid sequence | NO | Nucleotide sequence |
| Anti- | 386 | Gln Val Gln Leu Val | | |
| PD-1 | | Gln Ser Gly Ala Glu | | |
| heavy | | Val Lys Lys Pro Gly | | |
| chain | | Ser Ser Val Lys Val | | |
| | | Ser Cys Lys Ala Ser | | |
| | | Gly Gly Thr Phe Ser | | |
| | | Ser Tyr Ala Ile Ser | | |
| | | Trp Val Arg Gln Ala | | |
| | | Pro Gly Gln Gly Leu | | |
| | | Glu Trp Met Gly Gly | | |
| | | Ile Ile Pro Ile Phe Asp | | |
| | | Thr Ala Asn Tyr Ala | | |
| | | Gln Lys Phe Gln Gly | | |
| | | Arg Val Thr Ile Thr | | |
| | | Ala Asp Glu Ser Thr | | |
| | | Ser Thr Ala Tyr Met | | |
| | | Glu Leu Ser Ser Leu | | |
| | | Arg Ser Glu Asp Thr | | |
| | | Ala Val Tyr Tyr Cys | | |
| | | Ala Arg Pro Gly Leu | | |
| | | Ala Ala Ala Tyr Asp | | |
| | | Thr Gly Ser Leu Asp | | |
| | | Tyr Trp Gly Gln Gly | | |
| | | Thr Leu Val Thr Val | | |
| | | Ser Ser Ala Ser Thr | | |
| | | Lys Gly Pro Ser Val | | |
| | | Phe Pro Leu Ala Pro | | |
| | | Cys Ser Arg Ser Thr | | |
| | | Ser Glu Ser Thr Ala | | |
| | | Ala Leu Gly Cys Leu | | |
| | | Val Lys Asp Tyr Phe | | |
| | | Pro Glu Pro Val Thr | | |
| | | Val Ser Trp Asn Ser | | |
| | | Gly Ala Leu Thr Ser | | |
| | | Gly Val His Thr Phe | | |
| | | Pro Ala Val Leu Gln | | |
| | | Ser Ser Gly Leu Tyr | | |
| | | Ser Leu Ser Ser Val | | |
| | | Val Thr Val Pro Ser | | |
| | | Ser Ser Leu Gly Thr | | |
| | | Lys Thr Tyr Thr Cys | | |
| | | Asn Val Asp His Lys | | |
| | | Pro Ser Asn Thr Lys | | |
| | | Val Asp Lys Arg Val | | |
| | | Glu Ser Lys Tyr Gly | | |
| | | Pro Pro Cys Pro Pro | | |
| | | Cys Pro Ala Pro Glu | | |
| | | Phe Leu Gly Gly Pro | | |
| | | Ser Val Phe Leu Phe | | |
| | | Pro Pro Lys Pro Lys | | |
| | | Asp Thr Leu Met Ile | | |
| | | Ser Arg Thr Pro Glu | | |
| | | Val Thr Cys Val Val | | |
| | | Val Asp Val Ser Gln | | |
| | | Glu Asp Pro Glu Val | | |
| | | Gln Phe Asn Trp Tyr | | |
| | | Val Asp Gly Val Glu | | |
| | | Val His Asn Ala Lys | | |
| | | Thr Lys Pro Arg Glu | | |
| | | Glu Gln Phe Asn Ser | | |
| | | Thr Tyr Arg Val Val | | |
| | | Ser Val Leu Thr Val | | |
| | | Leu His Gln Asp Trp | | |
| | | Leu Asn Gly Lys Glu | | |
| | | Tyr Lys Cys Lys Val | | |
| | | Ser Asn Lys Gly Leu | | |
| | | Pro Ser Ser Ile Glu | | |
| | | Lys Thr Ile Ser Lys | | |
| | | Ala Lys Gly Gln Pro | | |
| | | Arg Glu Pro Gln Val | | |
| | | Tyr Thr Leu Pro Pro | | |
| | | Ser Gln Glu Glu Met | | |
| | | Thr Lys Asn Gln Val | | |
| | | Ser Leu Thr Cys Leu | | |
| | | Val Lys Gly Phe Tyr | | |
| | | Pro Ser Asp Ile Ala | | |
| | | Val Glu Trp Glu Ser | | |
| | | Asn Gly Gln Pro Glu | | |
| | | Asn Asn Tyr Lys Thr | | |
| | | Thr Pro Pro Val Leu | | |
| | | Asp Ser Asp Gly Ser | | |
| | | Phe Phe Leu Tyr Ser | | |
| | | Arg Leu Thr Val Asp | | |
| | | Lys Ser Arg Trp Gln | | |
| | | Glu Gly Asn Val Phe | | |
| | | Ser Cys Ser Val Met | | |
| | | His Glu Ala Leu His | | |
| | | Asn His Tyr Thr Gln | | |
| | | Lys Ser Leu Ser Leu | | |
| | | Ser Leu Gly Lys | | |

### Table 25. Exemplary Anti-PD-1 Light Chain Sequence

Table 25 provides an exemplary sequences for use in an anti-PD-1 light chain.

| | SEQ | | SEQ | |
|---|---|---|---|---|
| | ID | | ID | |
| Name | NO | Amino acid sequence | NO | Nucleotide sequence |
| Anti- | 387 | Glu Ile Val Leu Thr | | |
| PD-1 | | Gln Ser Pro Ala Thr | | |
| light | | Leu Ser Leu Ser Pro | | |
| chain | | Gly Glu Arg Ala Thr | | |
| | | Leu Ser Cys Arg Ala | | |
| | | Ser Gln Ser Val Arg | | |
| | | Ser Tyr | | |
| | | Leu Ala Trp Tyr Gln | | |
| | | Gln Lys Pro Gly Gln | | |
| | | Ala Pro Arg Leu Leu | | |
| | | Ile Tyr Asp Ala Ser | | |
| | | Asn Arg Ala Thr Gly | | |
| | | Ile Pro Ala Arg Phe | | |
| | | Ser Gly Ser Gly Ser | | |
| | | Gly Thr Asp Phe Thr | | |
| | | Leu Thr Ile Ser Ser | | |
| | | Leu Glu Pro Glu Asp | | |
| | | Phe Ala Val Tyr Tyr | | |
| | | Cys Gln Gln Arg Asn | | |
| | | Tyr Trp Pro Leu Thr | | |
| | | Phe Gly Gln Gly Thr | | |
| | | Lys Val Glu Ile Lys | | |
| | | Arg Thr Val Ala Ala | | |
| | | Pro Ser Val Phe Ile | | |
| | | Phe Pro Pro Ser Asp | | |
| | | Glu Gln Leu Lys Ser | | |
| | | Gly Thr Ala Ser Val | | |
| | | Val Cys Leu Leu Asn | | |
| | | Asn Phe Tyr Pro Arg | | |
| | | Glu Ala Lys Val Gln | | |
| | | Trp Lys Val Asp Asn | | |
| | | Ala Leu Gln Ser Gly | | |
| | | Asn Ser Gln Glu Ser | | |
| | | Val Thr Glu Gln Asp | | |
| | | Ser Lys Asp Ser Thr | | |
| | | Tyr Ser Leu Ser Ser | | |
| | | Thr Leu Thr Leu Ser | | |
| | | Lys Ala Asp Tyr Glu | | |
| | | Lys His Lys Val Tyr | | |
| | | Ala Cys Glu Val Thr | | |
| | | His Gln Gly Leu Ser | | |
| | | Ser Pro Val Thr Lys | | |
| | | Ser Phe Asn Arg Gly | | |
| | | Glu Cys | | |

### Table 26. Amino Acid and Nucleic Acid Sequences for Human PD-1

Table 26 provides the amino acid sequence and an exemplary nucleic acid sequence for human PD-1. The transmembrane domain, located at residues 170 to 191 (and underlined for emphasis) comprises the amino acids: Val Val Gly Val Val Gly Gly Leu Leu Gly Ser Leu Val Leu Leu Val Trp Val Leu Ala Val Ile (SEQ ID NO: 564).

| | SEQ | | SEQ | |
|---|---|---|---|---|
| | ID | | ID | |
| Name | NO | Amino acid sequence | NO | Nucleotide sequence |
| Human | 388 | Phe Leu Asp Ser Pro | 389 | |
| PD-1 | | Asp Arg Pro Trp Asn | | |
| | | Pro Pro Thr Phe Ser | | |
| | | Pro Ala Leu Leu Val | | |
| | | Val Thr Glu Gly Asp | | |
| | | Asn Ala Thr Phe Thr | | |
| | | Cys Ser Phe Ser Asn | | |
| | | Thr Ser Glu Ser Phe | | |
| | | Val Leu Asn Trp Tyr | | |
| | | Arg Met Ser Pro Ser | | |
| | | Asn Gln Thr Asp Lys | | |
| | | Leu Ala Ala Phe Pro | | |
| | | Glu Asp Arg Ser Gln | | |
| | | Pro Gly Gln Asp Cys | | |
| | | Arg Phe Arg Val Thr | | |
| | | Gln Leu Pro Asn Gly | | |
| | | Arg Asp Phe His Met | | |
| | | Ser Val Val Arg Ala | | |
| | | Arg Arg Asn Asp Ser | | |
| | | Gly Thr Tyr Leu Cys | | |
| | | Gly Ala Ile Ser Leu | | |
| | | Ala Pro Lys Ala Gln | | |
| | | Ile Lys Glu Ser Leu | | |
| | | Arg Ala Glu Leu Arg | | |
| | | Val Thr Glu Arg Arg | | |
| | | Ala Glu Val Pro Thr | | |
| | | Ala His Pro Ser Pro | | |
| | | Ser Pro Arg Pro Ala | | |
| | | Gly Gln Phe Gln Thr | | |
| | | Leu Val Val Gly Val | | |
| | | Val Gly Gly Leu Leu | | |
| | | Gly Ser Leu Val Leu | | |
| | | Leu Val Trp Val Leu | | |
| | | Ala Val Ile Cys Ser | | |
| | | Arg Ala Ala Arg Gly | | |
| | | Thr Ile Gly Ala Arg | | |
| | | Arg Thr Gly Gln Pro | | |
| | | Leu Lys Glu Asp Pro | | |
| | | Ser Ala Val Pro Val | | |
| | | Phe Ser Val Asp Tyr | | |
| | | Gly Glu Leu Asp Phe | | |
| | | Gln Trp Arg Glu Lys | | |
| | | Thr Pro Glu Pro Pro | | |
| | | Val Pro Cys Val Pro | | |
| | | Glu Gln Thr Glu Tyr | | |
| | | Ala Thr Ile Val Phe | | |
| | | Pro Ser Gly Met Gly | | |
| | | Thr Ser Ser Pro Ala | | |
| | | Arg Arg Gly Ser Ala | | |
| | | Asp Gly Pro Arg Ser | | |
| | | Ala Gln Pro Leu Arg | | |
| | | Pro Glu Asp Gly His | | |

### Table 27. Exemplary Anti-MUC16 Variable Heavy (VH) and Variable Light (VL) Sequences

Table 27 provides the amino acid sequence and nucleic acid sequences for exemplary anti-MUC16 VH and VL sequences. The CDRs are underlined.

| | SEQ | | SEQ | |
|---|---|---|---|---|
| | ID | | ID | |
| Name | NO | Amino acid sequence | NO | Nucleotide sequence |
| Anti- | 390 | QVQLQESGPGLVK | 404 | CAGGTGCAACTGCAGGAATCAGGTCC |
| MUC16 | | PSQTLSLTCTVSGY | | AGGCTTGGTCAAGCCATCGCAGACTC |
| VHI | | SIVSHYYWSWIRQ | | TTAGTCTGACATGCACCGTGAGTGGC |
| | | HPGKGLEWIGYISS | | TATAGCATCGTGTCGCACTATTATTGG |
| | | DGSNYYNPSLKSL | | TCTTGGATCAGGCAGCATCCAGGAAA |
| | | VTISVDTSKNQFSL | | GGGACTGGAGTGGATCGGGTACATTA |
| | | KLSSVTAADTAVY | | GCAGCGATGGGAGCAACTATTACAAC |
| | | YCVRGVDYWGQG | | CCATCTCTGAAGTCCCTGGTAACTATT |
| | | TMVTVSS | | AGCGTGGATACAAGCAAAAATCAGTT |
| | | | | TTCATTAAAGCTCTCTTCAGTGACCGC |
| | | | | AGCTGATACCGCCGTCTATTATTGCGT |
| | | | | GCGGGGGGTGGACTACTGGGGTCAGG |
| | | | | GCACCATGGTTACTGTGTCATCA |
| Anti- | 391 | QVQLQESGPGLVK | 405 | CAGGTACAGCTGCAGGAGAGTGGCCC |
| MUC16 | | PSDTLSLTCAVSGY | | TGGTTTAGTAAAGCCATCAGATACAC |
| VH2 | | SIVSHYYWGWIRQ | | TTTCACTTACCTGCGCCGTGTCTGGTT |
| | | PPGKGLEWIGYISS | | ATTCTATCGTGAGCCACTATTACTGGG |
| | | DGSNYYNPSLKSR | | GATGGATCCGCCAGCCCCCTGGCAAA |
| | | VTMSVDTSKNQFS | | GGTCTTGAGTGGATTGGCTATATAAG |
| | | LKLSSVTAVDTAV | | TTCGGATGGCAGTAACTATTACAATC |
| | | YYCVRGVDYWGQ | | CTTCTCTGAAGAGCCGTGTCACTATGA |
| | | GTMVTVSS | | GCGTGGACACTAGCAAAAACCAGTTC |
| | | | | AGCCTGAAGCTGTCCTCCGTCACCGC |
| | | | | CGTAGACACCGCTGTCTACTATTGTGT |
| | | | | TAGGGGGGTGGACTACTGGGGCCAAG |
| | | | | GCACCATGGTCACGGTGAGCAGC |
| Anti- | 392 | EVQLVESGGGLVQ | | |
| MUC16 | | PGGSLRLSCAASGY | | |
| VH3 | | SIVSHYYMSWVRQ | | |
| | | APGKGLEWVSVISS | | |
| | | DGSNYYADSVKGR | | |
| | | FTISRDNSKNTLYL | | |
| | | QMNSLRAEDTAVY | | |
| | | YCVRGVDYWGQG | | |
| | | TLVTVSS | | |
| Anti- | 393 | EVQLVESGGGLVQ | 406 | GAGGTGCAGCTCGTCGAGTCCGGAGG |
| MUC16 | | PGRSLRLSCAASGY | | CGGTCTGGTGCAACCCGGCCGTTCTTT |
| VH4 | | SIVSHYYMHWVRQ | | GCGGCTGAGTTGCGCTGCCAGTGGGT |
| | | APGKGLEWVSAISS | | ATAGCATCGTGAGTCACTATTACATG |
| | | DGSNEYADSVEGR | | CATTGGGTTCGTCAAGCCCCTGGCAA |
| | | FTISRDNAKNSLYL | | GGGACTAGAGTGGGTGTCCGCCATCT |
| | | QMNSLRAEDTAVY | | CCTCAGACGGTAGTAATGAGTACGCG |
| | | YCVRGVDYWGQG | | GACAGCGTAGAGGGTAGATTCACCAT |
| | | TLVTVSS | | TTCTCGGGACAATGCCAAAAATAGTT |
| | | | | ATACCTCCAAATGAATTCCCTTAGGG |
| | | | | CCGAAGACACTGCCGTGTACTACTGT |
| | | | | GTTCGGGGCGTGGACTACTGGGGGCA |
| | | | | GGGGACATTGGTGACTGTGAGCTCA |
| Anti- | 394 | QVQLQESGPGLVK | 407 | CAGGTCCAACTGCAGGAATCTGGCCC |
| MUC16 | | PSQTLSLTCTVSGY | | CGGACTGGTTAAACCATCTCAGACAC |
| VH5 | | SIVSHYYWSWIRQ | | TCTCCCTGACCTGCACCGTGTCTGGAT |
| | | HPGKGLEWIGYISS | | ACAGCATCGTTTCTCATTATTACTGGT |
| | | DGSNEYNPSLKSLV | | CATGGATTAGGCAGCATCCCGGAAAA |
| | | TISVDTSKNQFSLK | | GGGCTTGAATGGATTGGCTACATCTC |
| | | LSSVTAADTAVYF | | CTCCGACGGCTCCAATGAGTACAACC |
| | | CVRGVDYWGQGT | | CATCACTTAAATCTCTGGTCACGATAA |
| | | MVTVSS | | GCGTAGACACATCTAAAAATCAGTTC |
| | | | | TCATTAAAGCTCAGCTCTGTTACAGCT |
| | | | | GCCGACACCGCTGTGTACTTCTGTGTG |
| | | | | CGAGGGGTTGACTACTGGGGGCAGGG |
| | | | | CACAATGGTGACAGTGTCTTCC |
| Anti- | 395 | QVQLVQSGAEVKK | 408 | CAGGTTCAACTGGTTCAGTCCGGAGC |
| MUC16 | | PGSSVKVSCKASG | | CGAGGTCAAAAAGCCTGGATCCTCTG |
| VH6 | | YSIVSHYYISWVRQ | | TGAAGGTGTCATGTAAGGCTTCTGGC |
| | | APGQGLEWMGGIS | | TACAGCATCGTCTCACATTATTACATA |
| | | SDGSNNYAQKFQG | | TCTTGGGTGCGACAGGCCCCCGGCCA |
| | | RVTITADESTSTAY | | GGGGCTCGAGTGGATGGGAGGTATTT |
| | | MELSSLRSEDTAVY | | CCTCCGACGGGAGTAACAATTACGCT |
| | | YCVRGVDYWGQG | | CAGAAATTTCAGGGCCGGGTGACCAT |
| | | TLVTVSS | | TACCGCCGACGAAAGTACAAGCACCG |
| | | | | CTTATATGGAATTAAGCTCTTTAAGAT |
| | | | | CAGAGGACACGGCTGTGTACTACTGT |
| | | | | GTAAGGGGCGTGGATTACTGGGGTCA |
| | | | | GGGGACGCTCGTCACCGTCTCGAGC |
| Anti- | 396 | QVQLQESGPGLVK | 409 | CAGGTCCAGCTCCAGGAATCCGGCCC |
| MUC16 | | PSETLSLTCTVSGY | | AGGGTTGGTGAAGCCTTCGGAGACCC |
| VH7 | | SIVSHYYWSWIRQP | | TGTCTCTGACATGCACAGTCAGCGGC |
| | | PGKGLEWIGYISSD | | TATAGTATCGTCTCCCACTATTATTGG |
| | | GSNNYNPSLKSRVT | | TCTTGGATTCGGCAACCTCCAGGCAA |
| | | ISVDTSKNQFSLKL | | GGGGTTAGAATGGATTGGATACATCT |
| | | SSVTAADTAVYYC | | CAAGCGATGGGTCCAATAACTACAAC |
| | | VRGVDYWGQGTT | | CCAAGTCTCAAAAGTAGAGTGACTAT |
| | | VT | | CTCTGTGGATACCAGTAAAAACCAGT |
| | | VSS | | TTTCACTCAAGTTGAGTTCCGTCACCG |
| | | | | CCGCCGACACAGCCGTTTACTACTGT |
| | | | | GTTCGGGGAGTGGACTACTGGGGCCA |
| | | | | AGGTACCACGGTTACCGTGAGCAGC |
| Anti- | 397 | QVQLQESGPGLVK | 410 | CAGGTGCAGCTGCAGGAGAGCGGCCC |
| MUC16 | | PSDTLSLTCAVSGY | | CGGCCTGGTGAAGCCCAGCGACACCC |
| VH8 | | SIVSHYYWHWIRQ | | TGAGCCTGACCTGCGCCGTGAGCGGC |
| | | PPGKGLEWMGYIS | | TACAGCATCGTGAGCCACTACTACTG |
| | | SDGSNDFNPSLKTR | | GCACTGGATCAGACAGCCCCCCGGCA |
| | | ITISRDTSKNQFSLK | | AGGGCCTGGAGTGGATGGGCTACATC |
| | | LSSVTAVDTAVYY | | AGCAGCGACGGCAGCAACGACTTCAA |
| | | CVRGVDYWGQGT | | CCCCAGCCTGAAGACCAGAATCACCA |
| | | LVTVSS | | TCAGCAGAGACACCAGCAAGAACCAG |
| | | | | TTCAGCCTGAAGCTGAGCAGCGTGAC |
| | | | | CGCCGTGGACACCGCCGTGTACTACT |
| | | | | GCGTGAGAGGCGTGGACTACTGGGGC |
| | | | | CAGGGCACCCTGGTGACCGTGAGCAG |
| | | | | C |
| Anti- | 398 | QVQLQESGPGLVK | 411 | CAGGTGCAGCTGCAGGAGAGCGGCCC |
| MUC16 | | PSQTLSLTCAVYGY | | CGGCCTGGTGAAGCCCAGCCAGACCC |
| VH9 | | SIVSHYYWSWIRQP | | TGAGCCTGACCTGCGCCGTGTACGGC |
| | | PGKGLEWIGEISSD | | TACAGCATCGTGAGCCACTACTACTG |
| | | GSNNYNPSLKSRVT | | GAGCTGGATCAGACAGCCCCCCGGCA |
| | | ISVDTSKNQFSLKL | | AGGGCCTGGAGTGGATCGGCGAGATC |
| | | SSVTAADTAVYYC | | AGCAGCGACGGCAGCAACAACTACAA |
| | | VRGVDYWGQGTL | | CCCCAGCCTGAAGAGCAGAGTGACCA |
| | | VTVSS | | TCAGCGTGGACACCAGCAAGAACCAG |
| | | | | TTCAGCCTGAAGCTGAGCAGCGTGAC |
| | | | | CGCCGCCGACACCGCCGTGTACTACT |
| | | | | GCGTGAGAGGCGTGGACTACTGGGGC |
| | | | | CAGGGCACCCTGGTGACCGTGAGCAG |
| | | | | C |
| Anti- | 399 | QVQLQESGPGLVK | 412 | CAGGTGCAGCTGCAGGAGAGCGGCCC |
| MUC16 | | PSETLSLTCAVSGY | | CGGCCTGGTGAAGCCCAGCGAGACCC |
| VH10 | | SIVSHYYWGWIRQ | | TGAGCCTGACCTGCGCCGTGAGCGGC |
| | | PPGKGLEWIGSISS | | TACAGCATCGTGAGCCACTACTACTG |
| | | DGSNYYNPSLKSR | | GGGCTGGATCAGACAGCCCCCCGGCA |
| | | VTISVDTSKNQFSL | | AGGGCCTGGAGTGGATCGGCAGCATC |
| | | KLSSVTAADTAVY | | AGCAGCGACGGCAGCAACTACTACAA |
| | | YCVRGVDYWGQG | | CCCCAGCCTGAAGAGCAGAGTGACCA |
| | | TLVTVSS | | TCAGCGTGGACACCAGCAAGAACCAG |
| | | | | TTCAGCCTGAAGCTGAGCAGCGTGAC |
| | | | | CGCCGCCGACACCGCCGTGTACTACT |
| | | | | GCGTGAGAGGCGTGGACTACTGGGGC |
| | | | | CAGGGCACCCTGGTGACCGTGAGCAG |
| | | | | C |
| Anti- | 400 | QVQLVESGGGVVQ | 413 | CAGGTGCAGCTGGTGGAGAGCGGCGG |
| MUC16 | | PGRSLRLSCAASGY | | CGGCGTGGTGCAGCCCGGCAGAAGCC |
| VH11 | | SIVSHYYWNWVRQ | | TGAGACTGAGCTGCGCCGCCAGCGGC |
| | | APGKGLEWVAYIS | | TACAGCATCGTGAGCCACTACTACTG |
| | | SDGSNEYNPSLKNR | | GAACTGGGTGAGACAGGCCCCCGGCA |
| | | FTISRDNSKNTLYL | | AGGGCCTGGAGTGGGTGGCCTACATC |
| | | QMNSLRAEDTAVY | | AGCAGCGACGGCAGCAACGAGTACA |
| | | YCVRGVDYWGQG | | ACCCCAGCCTGAAGAACAGATTCACC |
| | | TTVTVSS | | ATCAGCAGAGACAACAGCAAGAACA |
| | | | | CCCTGTACCTGCAGATGAACAGCCTG |
| | | | | AGAGCCGAGGACACCGCCGTGTACTA |
| | | | | CTGCGTGAGAGGCGTGGACTACTGGG |
| | | | | GCCAGGGCACCACCGTGACCGTGAGC |
| | | | | AGC |
| Anti- | 401 | QVQLQESGPGLVK | 414 | CAGGTGCAGCTGCAGGAGAGCGGCCC |
| MUC16 | | PSQTLSLTCTVSGY | | CGGCCTGGTGAAGCCCAGCCAGACCC |
| VH12 | | SIVSHYYWNWIRQ | | TGAGCCTGACCTGCACCGTGAGCGGC |
| | | HPGKGLEWIGYISS | | TACAGCATCGTGAGCCACTACTACTG |
| | | DGSNEYNPSLKNL | | GAACTGGATCAGACAGCACCCCGGCA |
| | | VTISVDTSKNQFSL | | AGGGCCTGGAGTGGATCGGCTACATC |
| | | KLSSVTAADTAVY | | AGCAGCGACGGCAGCAACGAGTACA |
| | | YCVRGVDYWGQG | | ACCCCAGCCTGAAGAACCTGGTGACC |
| | | TMVTVSS | | ATCAGCGTGGACACCAGCAAGAACCA |
| | | | | GTTCAGCCTGAAGCTGAGCAGCGTGA |
| | | | | CCGCCGCCGACACCGCCGTGTACTAC |
| | | | | TGCGTGAGAGGCGTGGACTACTGGGG |
| | | | | CCAGGGCACCATGGTGACCGTGAGCA |
| | | | | GC |
| Anti- | 402 | QVQLQESGPGLVK | 415 | CAGGTGCAGCTGCAGGAGAGCGGCCC |
| MUC16 | | PSDTLSLTCAVSGY | | CGGCCTGGTGAAGCCCAGCGACACCC |
| VH13 | | SIVSHYYWNWIRQ | | TGAGCCTGACCTGCGCCGTGAGCGGC |
| | | PPGKGLEWIGYISS | | TACAGCATCGTGAGCCACTACTACTG |
| | | DGSNEYNPSLKNR | | GAACTGGATCAGACAGCCCCCCGGCA |
| | | VTMSVDTSKNQFS | | AGGGCCTGGAGTGGATCGGCTACATC |
| | | LKLSSVTAVDTAV | | AGCAGCGACGGCAGCAACGAGTACA |
| | | YYCVRGVDYWGQ | | ACCCCAGCCTGAAGAACAGAGTGACC |
| | | GTMVTVSS | | ATGAGCGTGGACACCAGCAAGAACCA |
| | | | | GTTCAGCCTGAAGCTGAGCAGCGTGA |
| | | | | CCGCCGTGGACACCGCCGTGTACTAC |
| | | | | TGCGTGAGAGGCGTGGACTACTGGGG |
| | | | | CCAGGGCACCATGGTGACCGTGAGCA |
| | | | | GC |
| Anti- | 403 | EVQLLESGGGLVQ | 416 | GAGGTGCAGCTGCTGGAGAGCGGCGG |
| MUC16 | | PGGSLRLSCAASGY | | CGGCCTGGTGCAGCCCGGCGGCAGCC |
| VH14 | | SIVSHYYWNWVRQ | | TGAGACTGAGCTGCGCCGCCAGCGGC |
| | | APGKGLEWVSYISS | | TACAGCATCGTGAGCCACTACTACTG |
| | | DGSNEYNPSLKNRF | | GAACTGGGTGAGACAGGCCCCCGGCA |
| | | TISRDNSKNTLYLQ | | AGGGCCTGGAGTGGGTGAGCTACATC |
| | | MNSLRAEDTAVYY | | AGCAGCGACGGCAGCAACGAGTACA |
| | | CVRGVDYWGQGT | | ACCCCAGCCTGAAGAACAGATTCACC |
| | | LVTVSS | | ATCAGCAGAGACAACAGCAAGAACA |
| | | | | CCCTGTACCTGCAGATGAACAGCCTG |
| | | | | AGAGCCGAGGACACCGCCGTGTACTA |
| | | | | CTGCGTGAGAGGCGTGGACTACTGGG |
| | | | | GCCAGGGCACCCTGGTGACCGTGAGC |
| | | | | AGC |
| Anti- | 417 | DIQMTQSPSSLSAS | 429 | GACATACAGATGACTCAGAGCCCCTC |
| MUC16 | | VGDRVTITCQASRD | | CTCACTCTCGGCATCAGTCGGCGACA |
| VL1 | | INNFLNWYQQKPG | | GGGTCACAATTACCTGTCAGGCTTCTC |
| | | KAPKLLIYRANNLE | | GCGACATTAATAACTTCCTGAATTGGT |
| | | TGVPSRFSGSGSGT | | ATCAGCAAAAGCCCGGGAAGGCCCCT |
| | | DFTFTISSLQPEDIA | | AAGCTGTTGATTTATAGAGCAAATAA |
| | | TYFCLQYGDLYTF | | TCTCGAAACCGGCGTGCCCAGTAGGT |
| | | GGGTKVEIK | | TTAGCGGGTCCGGGAGCGGAACAGAC |
| | | | | TTCACATTCACCATTTCTAGTTTGCAG |
| | | | | CCCGAAGACATTGCTACATATTTTTGC |
| | | | | CTGCAGTACGGGGATCTCTACACTTTC |
| | | | | GGGGGCGGAACAAAGGTTGAGATAA |
| | | | | AA |
| Anti- | 418 | DIQMTQSPSSLSAS | 430 | GATATTCAAATGACGCAGTCACCCTC |
| MUC16 | | VGDRVTITCQASRD | | ATCGCTCTCTGCGTCAGTAGGGGATC |
| VL2 | | INNFLNWYQQKPG | | GTGTCACGATAACCTGTCAAGCATCA |
| | | KAPKLLIYRANNLE | | AGGGACATCAACAACTTCCTCAACTG |
| | | TGVPSRFSGSGSGT | | GTACCAACAGAAGCCTGGCAAGGCAC |
| | | DFTFTISSLQPEDIA | | CTAAACTCCTGATCTACCGGGCTAAC |
| | | TYYCLQYGDLYTF | | AACCTAGAAACCGGGGTTCCGAGCCG |
| | | GGGTKVEIK | | ATTCAGTGGGTCTGGAAGCGGGACAG |
| | | | | ACTTTACGTTCACTATTAGTTCGCTAC |
| | | | | AGCCCGAAGACATTGCGACATATTAC |
| | | | | TGTCTTCAGTATGGGGATTTGTATACC |
| | | | | TTTGGGGGAGGCACCAAGGTAGAGAT |
| | | | | AAAG |
| Anti- | 419 | DIQMTQSPSSLSAS | 431 | GACATCCAGATGACTCAGAGCCCGTC |
| MUC16 | | VGDRVTITCRASRD | | TTCTCTATCCGCAAGTGTAGGCGATCG |
| VL3 | | INNFLGWYQQKPG | | TGTCACCATCACATGCCGGGCTTCCCG |
| | | KAPKRLIYRANSLQ | | GGATATCAACAACTTCCTTGGGTGGT |
| | | SGVPSRFSGSGSGT | | ATCAGCAGAAGCCCGGAAAAGCCCCC |
| | | EFTLTISSLQPEDFA | | AAACGGCTCATCTACAGAGCGAATTC |
| | | TYYCLQYGDLYTF | | CCTGCAGTCAGGTGTCCCCAGTAGGT |
| | | GQGTKVEIK | | TCAGCGGATCAGGCTCGGGGACCGAA |
| | | | | TTCACTCTGACCATTAGCTCACTGCAG |
| | | | | CCTGAGGATTTCGCTACTTACTATTGC |
| | | | | CTGCAATACGGCGATCTGTACACTTTC |
| | | | | GGGCAGGGCACCAAGGTGGAAATAA |
| | | | | AA |
| Anti- | 420 | EIVLTQSPGTLSLSP | 432 | GAAATCGTACTGACCCAGTCTCCCGG |
| MUC16 | | GERATLSCRASRDI | | AACCCTGAGTCTCTCACCCGGCGAGC |
| VL4 | | NNFLAWYQQKPGQ | | GCGCAACACTGTCGTGTAGGGCCAGT |
| | | APRLLIYRANSRAT | | AGGGACATAAATAACTTCCTAGCCTG |
| | | GIPDRFSGSGSGTD | | GTACCAACAAAAACCGGGTCAGGCTC |
| | | FTLTISRLEPEDFAV | | CAAGACTGTTGATCTATAGAGCTAAC |
| | | YYCLQYGDLYTFG | | TCCAGGGCCACCGGCATCCCAGACCG |
| | | QGTKVEIK | | ATTCTCAGGCTCCGGATCTGGAACCG |
| | | | | ACTTCACGCTCACCATTAGCCGACTA |
| | | | | GAACCTGAGGACTTTGCTGTATACTAT |
| | | | | TGCCTGCAGTACGGCGACCTGTATAC |
| | | | | CTTTGGACAGGGTACCAAGGTCGAGA |
| | | | | TCAAG |
| Anti- | 421 | EIVLTQSPATLSLSP | 433 | GAGATCGTACTTACGCAGAGCCCAGC |
| MUC16 | | GERATLSCRASRDI | | AACTCTGTCTCTGTCCCCCGGAGAAC |
| VL5 | | NNFLAWYQQKPGQ | | GGGCCACCCTGTCGTGCCGGGCCAGC |
| | | APRLLIYRANNRAT | | CGTGATATTAATAATTTCCTGGCCTGG |
| | | GIPARFSGSGPGTD | | TATCAACAAAAACCGGGGCAGGCTCC |
| | | FTLTISSLEPEDFAV | | TCGACTGTTGATCTACCGGGCCAACA |
| | | YYCLQYGDLYT | | ATAGAGCAACTGGTATCCCTGCTCGC |
| | | FGGGTKVEIK | | TTCTCCGGCAGTGGGCCAGGTACAGA |
| | | | | CTTCACCCTGACTATTTCGTCACTCGA |
| | | | | ACCAGAAGACTTTGCCGTGTATTATTG |
| | | | | CTTACAATACGGGGATCTGTACACTTT |
| | | | | CGGAGGAGGAACTAAGGTCGAAATTA |
| | | | | AG |
| Anti- | 422 | EIVLTQSPDFQSVTP | 434 | GAGATCGTGCTGACCCAGAGCCCCGA |
| MUC16 | | KEKVTITCRASRDI | | CTTCCAGAGCGTGACCCCCAAGGAGA |
| VL6 | | NNFLHWYQQKPDQ | | AGGTGACCATCACCTGCAGAGCCAGC |
| | | SPKLLIKRANQSFS | | AGAGACATCAACAACTTCCTGCACTG |
| | | GVPSRFSGSGSGTD | | GTACCAGCAGAAGCCCGACCAGAGCC |
| | | FTLTINSLEAEDAA | | CCAAGCTGCTGATCAAGAGAGCCAAC |
| | | TYYCLQYGDLYTF | | CAGAGCTTCAGCGGCGTGCCCAGCAG |
| | | GQGTKVEIK | | ATTCAGCGGCAGCGGCAGCGGCACCG |
| | | | | ACTTCACCCTGACCATCAACAGCCTG |
| | | | | GAGGCCGAGGACGCCGCCACCTACTA |
| | | | | CTGCCTGCAGTACGGCGACCTGTACA |
| | | | | CCTTCGGCCAGGGCACCAAGGTGGAG |
| | | | | ATCAAG |
| Anti- | 423 | DIQMTQSPSSLSAS | 435 | GACATCCAGATGACCCAGAGCCCCAG |
| MUC16 | | VGDRVTITCRASRD | | CAGCCTGAGCGCCAGCGTGGGCGACA |
| VL? | | INNFLAWFQQKPG | | GAGTGACCATCACCTGCAGAGCCAGC |
| | | KAPKSLIYRANSLQ | | AGAGACATCAACAACTTCCTGGCCTG |
| | | SGVPSRFSGSGSGT | | GTTCCAGCAGAAGCCCGGCAAGGCCC |
| | | DFTLTISSLQPEDFA | | CCAAGAGCCTGATCTACAGAGCCAAC |
| | | TYYCLQYGDLYTF | | AGCCTGCAGAGCGGCGTGCCCAGCAG |
| | | GGGTKVEIK | | ATTCAGCGGCAGCGGCAGCGGCACCG |
| | | | | ACTTCACCCTGACCATCAGCAGCCTG |
| | | | | CAGCCCGAGGACTTCGCCACCTACTA |
| | | | | CTGCCTGCAGTACGGCGACCTGTACA |
| | | | | CCTTCGGCGGCGGCACCAAGGTGGAG |
| | | | | ATCAAG |
| Anti- | 424 | DIQMTQSPSSLSAS | 436 | GACATCCAGATGACCCAGAGCCCCAG |
| MUC16 | | VGDRVTITCRASRD | | CAGCCTGAGCGCCAGCGTGGGCGACA |
| VL8 | | INNFLAWYQQKPG | | GAGTGACCATCACCTGCAGAGCCAGC |
| | | KAPKLLLYRANRL | | AGAGACATCAACAACTTCCTGGCCTG |
| | | ESGVPSRFSGSGSG | | GTACCAGCAGAAGCCCGGCAAGGCCC |
| | | TDYTLTISSLQPEDF | | CCAAGCTGCTGCTGTACAGAGCCAAC |
| | | ATYYCLQYGDLYT | | AGACTGGAGAGCGGCGTGCCCAGCAG |
| | | FGGGTKVEIK | | ATTCAGCGGCAGCGGCAGCGGCACCG |
| | | | | ACTACACCCTGACCATCAGCAGCCTG |
| | | | | CAGCCCGAGGACTTCGCCACCTACTA |
| | | | | CTGCCTGCAGTACGGCGACCTGTACA |
| | | | | CCTTCGGCGGCGGCACCAAGGTGGAG |
| | | | | ATCAAG |
| Anti- | 425 | DIQMTQSPSSLSAS | 437 | GACATCCAGATGACCCAGAGCCCCAG |
| MUC16 | | VGDRVTITCKASRD | | CAGCCTGAGCGCCAGCGTGGGCGACA |
| VL9 | | INNFLSWYQQKPG | | GAGTGACCATCACCTGCAAGGCCAGC |
| | | KAPKLLIYRANRLV | | AGAGACATCAACAACTTCCTGAGCTG |
| | | DGVPSRFSGSGSGT | | GTACCAGCAGAAGCCCGGCAAGGCCC |
| | | DFTFTISSLQPEDIA | | CCAAGCTGCTGATCTACAGAGCCAAC |
| | | TYYCLQYGDLYTF | | AGACTGGTGGACGGCGTGCCCAGCAG |
| | | GGGTKVEIK | | ATTCAGCGGCAGCGGCAGCGGCACCG |
| | | | | ACTTCACCTTCACCATCAGCAGCCTGC |
| | | | | AGCCCGAGGACATCGCCACCTACTAC |
| | | | | TGCCTGCAGTACGGCGACCTGTACAC |
| | | | | CTTCGGCGGCGGCACCAAGGTGGAGA |
| | | | | TCAAG |
| Anti- | 426 | DIQMTQSPSSLSAS | 438 | GACATCCAGATGACCCAGAGCCCCAG |
| MUC16 | | VGDRVTITCKASRD | | CAGCCTGAGCGCCAGCGTGGGCGACA |
| VL10 | | INNFLSWYQQKPG | | GAGTGACCATCACCTGCAAGGCCAGC |
| | | KAPKLLIYRANRLV | | AGAGACATCAACAACTTCCTGAGCTG |
| | | DGVPSRFSGSGSGT | | GTACCAGCAGAAGCCCGGCAAGGCCC |
| | | DFTLTISSLQPEDFA | | CCAAGCTGCTGATCTACAGAGCCAAC |
| | | TYYCLQYGDLYTF | | AGACTGGTGGACGGCGTGCCCAGCAG |
| | | GQGTKVEIK | | ATTCAGCGGCAGCGGCAGCGGCACCG |
| | | | | ACTTCACCCTGACCATCAGCAGCCTG |
| | | | | CAGCCCGAGGACTTCGCCACCTACTA |
| | | | | CTGCCTGCAGTACGGCGACCTGTACA |
| | | | | CCTTCGGCCAGGGCACCAAGGTGGAG |
| | | | | ATCAAG |
| Anti- | 427 | EIVLTQSPGTLSLSP | 439 | GAGATCGTGCTGACCCAGAGCCCCGG |
| MUC16 | | GERATLSCKASRDI | | CACCCTGAGCCTGAGCCCCGGCGAGA |
| VL11 | | NNFLSWYQQKPGQ | | GAGCCACCCTGAGCTGCAAGGCCAGC |
| | | APRLLIYRANRLVD | | AGAGACATCAACAACTTCCTGAGCTG |
| | | GIPDRFSGSGSGTD | | GTACCAGCAGAAGCCCGGCCAGGCCC |
| | | FTLTISRLEPEDFAV | | CCAGACTGCTGATCTACAGAGCCAAC |
| | | YYCLQYGDLYTF | | AGACTGGTGGACGGCATCCCCGACAG |
| | | GQGTKVEIK | | ATTCAGCGGCAGCGGCAGCGGCACCG |
| | | | | ACTTCACCCTGACCATCAGCAGACTG |
| | | | | GAGCCCGAGGACTTCGCCGTGTACTA |
| | | | | CTGCCTGCAGTACGGCGACCTGTACA |
| | | | | CCTTCGGCCAGGGCACCAAGGTGGAG |
| | | | | ATCAAG |
| Anti- | 428 | EIVLTQSPATLSLSP | 440 | GAGATCGTGCTGACCCAGAGCCCCGC |
| MUC16 | | GERATLSCKASRDI | | CACCCTGAGCCTGAGCCCCGGCGAGA |
| VL12 | | NNFLSWYQQKPGQ | | GAGCCACCCTGAGCTGCAAGGCCAGC |
| | | APRLLIYRANRLVD | | AGAGACATCAACAACTTCCTGAGCTG |
| | | GIPARFSGSGSGTD | | GTACCAGCAGAAGCCCGGCCAGGCCC |
| | | FTLTISSLEPEDFAV | | CCAGACTGCTGATCTACAGAGCCAAC |
| | | YYCLQYGDLYTF | | AGACTGGTGGACGGCATCCCCGCCAG |
| | | GQGTKVEIK | | ATTCAGCGGCAGCGGCAGCGGCACCG |
| | | | | ACTTCACCCTGACCATCAGCAGCCTG |
| | | | | GAGCCCGAGGACTTCGCCGTGTACTA |
| | | | | CTGCCTGCAGTACGGCGACCTGTACA |
| | | | | CCTTCGGCCAGGGCACCAAGGTGGAG |
| | | | | ATCAAG |

### Table 28. Exemplary Anti-MUC16 HC-Linker-TGFβRII ECD Sequences

Table 28 provides exemplary anti-MUC16 HC-linker-TGFβRII ECD sequences. Such heavy chain polypeptides can be paired with an anti-MUC16 light chain, e.g., SEQ ID NO: 443, to make an anti-MUC16 fusion protein. Such heavy chain polypeptides can be paired with an anti-MUC16 variable region, e.g., SEQ ID NOs: 417-428, to make an anti-MUC16 fusion protein.

| | SEQ | | SEQ | |
|---|---|---|---|---|
| | ID | | ID | |
| Name | NO | Amino acid sequence | NO | Nucleotide sequence |
| Anti- | 441 | QVQLQESGPGLVK | 442 | CAGGTGCAGCTGCAGGAGAGCGGCCC |
| MUC16 | | PSETLSLTCAVSGY | | CGGCCTGGTGAAGCCCAGCGAGACCC |
| VH10- | | SIVSHYYWGWIRQ | | TGAGCCTGACCTGCGCCGTGAGCGGC |
| IgG1(wt)- | | PPGKGLEWIGSISS | | TACAGCATCGTGAGCCACTACTACTG |
| linker- | | DGSNYYNPSLKSR | | GGGCTGGATCAGACAGCCCCCCGGCA |
| TGFβRII | | VTISVDTSKNQFSL | | AGGGCCTGGAGTGGATCGGCAGCATC |
| ECD | | KLSSVTAADTAVY | | AGCAGCGACGGCAGCAACTACTACAA |
| | | YCVRGVDYWGQG | | CCCCAGCCTGAAGAGCAGAGTGACCA |
| | | TLVTVSSASTKGPS | | TCAGCGTGGACACCAGCAAGAACCAG |
| | | VFPLAPSSKSTSGG | | TTCAGCCTGAAGCTGAGCAGCGTGAC |
| | | TAALGCLVKDYFP | | CGCCGCCGACACCGCCGTGTACTACT |
| | | EPVTVSWNSGALT | | GCGTGAGAGGCGTGGACTACTGGGGC |
| | | SGVHTFPAVLQSSG | | CAGGGCACCCTGGTGACCGTGAGCAG |
| | | LYSLSSVVTVPSSS | | CGCTAGCACCAAGGGCCCATCGGTCT |
| | | LGTQTYICNVNHKP | | TCCCCCTGGCACCCTCCTCCAAGAGC |
| | | SNTKVDKKVEPKS | | ACCTCTGGGGGCACAGCGGCCCTGGG |
| | | CDKTHTCPPCPAPE | | CTGCCTGGTCAAGGACTACTTCCCCG |
| | | LLGGPSVFLFPPKP | | AACCGGTGACGGTGTCGTGGAACTCA |
| | | KDTLMISRTPEVTC | | GGCGCCCTGACCAGCGGCGTGCACAC |
| | | VVVDVSHEDPEVK | | CTTCCCGGCTGTCCTACAGTCCTCAGG |
| | | FNWYVDGVEVHN | | ACTCTACTCCCTCAGCAGCGTGGTGA |
| | | AKTKPREEQYNST | | CCGTGCCCTCCAGCAGCTTGGGCACC |
| | | YRVVSVLTVLHQD | | CAGACCTACATCTGCAACGTGAATCA |
| | | WLNGKEYKCKVSN | | CAAGCCCAGCAACACCAAGGTGGACA |
| | | KALPAPIEKTISKA | | AGAAAGTGAGCCCAAATCTTGTGACA |
| | | KGQPREPQVYTLPP | | AAACTCACACATGCCCACCGTGCCCA |
| | | SREEMTKNQVSLT | | GCACCTGAACTCCTGGGGGGACCGTC |
| | | CLVKGFYPSDIAVE | | AGTCTTCCTCTTCCCCCCAAAACCCAA |
| | | WESNGQPENNYKT | | GGACACCCTCATGATCTCCCGGACCC |
| | | TPPVLDSDGSFFLY | | CTGAGGTCACATGCGTGGTGGTGGAC |
| | | SKLTVDKSRWQQG | | GTGAGCCACGAAGACCCTGAGGTCAA |
| | | NVFSCSVMHEALH | | GTTCAACTGGTACGTGGACGGCGTGG |
| | | NHYTQKSLSLSPGK | | AGGTGCATAATGCCAAGACAAAGCCG |
| | | QGGGSGGGGSIPPH | | CGGGAGGAGCAGTACAACAGCACGTA |
| | | VQKSVNNDMIVTD | | CCGTGTGGTCAGCGTCCTCACCGTCCT |
| | | NNGAVKFPQLCKF | | GCACCAGGACTGGCTGAATGGCAAGG |
| | | CDVRFSTCDNQKS | | AGTACAAGTGCAAGGTCTCCAACAAA |
| | | CMSNCSITSICEKPQ | | GCCCTCCCAGCCCCCATCGAGAAAAC |
| | | EVCVAVWRKNDE | | CATCTCCAAAGCCAAAGGGCAGCCCC |
| | | NITLETVCHDPKLP | | GAGAACCACAGGTGTACACCCTGCCC |
| | | YHDFILEDAASPKC | | CCATCCCGGGAGGAGATGACCAAGAA |
| | | IMKEKKKPGETFF | | CCAGGTCAGCCTGACCTGCCTGGTCA |
| | | MCSCSSDECNDNII | | AAGGCTTCTATCCCAGCGACATCGCC |
| | | FSEEYNTSNPD | | GTGGAGTGGGAGAGCAATGGGCAGCC |
| | | | | GGAGAACAACTACAAGACCACGCCTC |
| | | | | CCGTGCTGGACTCCGACGGCTCCTTCT |
| | | | | TCCTCTACAGCAAGCTCACCGTGGAC |
| | | | | AAGAGCAGGTGGCAGCAGGGGAACG |
| | | | | TCTTCTCATGCTCCGTGATGCATGAGG |
| | | | | CTCTGCACAACCACTACACGCAGAAG |
| | | | | AGCCTCTCCCTGTCTCCGGGTAAAGGT |
| | | | | GGAGGTGGTTCTGGAGGTGGAGGTAG |
| | | | | TATCCCTCCTCACGTACAGAAGTCCGT |
| | | | | GAACAATGACATGATTGTCACTGACA |
| | | | | ATAACGGAGCCGTCAAGTTTCCTCAG |
| | | | | CTATGTAAGTTCTGCGATGTTCGGTTC |
| | | | | TCCACATGCGATAATCAGAAAAGCTG |
| | | | | TATGTCTAATTGCAGTATCACTAGTAT |
| | | | | ATGCGAAAAACCTCAAGAAGTTTGCG |
| | | | | TCGCCGTGTGGCGGAAAAATGATGAA |
| | | | | AATATCACGCTTGAGACTGTCTGCCAT |
| | | | | GATCCAAAGTTACCCTACCACGACTT |
| | | | | CATCTTAGAAGACGCCGCATCACCCA |
| | | | | AGTGCATTATGAAAGAGAAAAAGAA |
| | | | | GCCAGGAGAAACATTCTTTATGTGCT |
| | | | | CATGCTCCTCTGACGAATGCAACGAC |
| | | | | AACATTATCTTCTCTGAGGAGTATAAC |
| | | | | ACCTCAAATCCAGACTGA |

### Table 29. Exemplary Anti-MUC16 LC Sequence

Table 29 provides exemplary anti-MUC16 LC sequences. Such polypeptides can be paired with an anti-MUC16 heavy chain, e.g., SEQ ID NO: 441, to make a MUC16 fusion protein. Such polypeptides can be paired with an anti-MUC16 variable heavy region, e.g., SEQ ID NOs: 390-403, to make a MUC16 fusion protein.

| | SEQ | |
|---|---|---|
| | ID | |
| Name | NO | Amino acid sequence |
| Anti- | 443 | DIQMTQSPSSLSASVGDRVTITCKASRDINNFLSWYQQKPG |
| MUC16 | | KAPKLLIYRANRLVDGVPSRFSGSGSGTDFTLTISSLQPEDF |
| VL10- | | ATYYCLQYGDLYTFGQGTKVEIKTVAAPSVFIFPPSDEQLK |
| KCL | | SGTASVVCLLNNFYPREAKVQWKVDNALQSGNSQESVTE |
| | | QDSKDSTYSLSSTLTLSKADYEKHKVYACEVTHQGLSSPV |
| | | TKSFNRGEC |

Table 30. Exemplary Anti-MUC1 V_{H} and V_{L} Sequences (CDRs Underlined)

Table 30 provides exemplary anti-MUC1 V_{H} and V_{L} sequences. The CDRs are underlined.

| | SEQ | |
|---|---|---|
| | ID | |
| Name | NO | Amino acid sequence |
| Anti- | 444 | QVQLVQSGAEVKKPGSSVKVSCKASGYAFSNFWMNWVR |
| MUC1 | | QAPGQGLEWMGQIYPGDGDTNYNGKFKGRVTITADKSTS |
| VH1 | | TAYMELSSLRSEDTAVYYCARSYYRSAWFAYWGQGTLVT |
| | | VSS |
| Anti- | 445 | QVQLVQSGAEVKKPGASVKVSCKASGYAFSNFWMNWVR |
| MUC1 | | QAPGQGLEWMGQIYPGDGDTNYNGKFKGRVTMTRDTSTS |
| VH2 | | TVYMELSSLRSEDTAVYYCARSYYRSAWFAYWGQGTLVT |
| | | VSS |
| Anti- | 446 | QVQLVQSGAEVKKPGASVKVSCKASGYAFSNFWMNWVR |
| MUC1 | | QAPGQGLEWMGQIYPGDGDTNYNGKFKGRVTMTRDTSIS |
| VH3 | | TAYMELSRLRSDDTAVYYCARSYYRSAWFAYWGQGTLVT |
| | | VSS |
| Anti- | 447 | QVQLVQSGAEVKKPGATVKISCKVSGYAFSNFWMNWVQ |
| MUC1 | | QAPGKGLEWMGQIYPGDGDTNYNGKFKGRVTITADTSTD |
| VH4 | | TAYMELSSLRSEDTAVYYCARSYYRSAWFAYWGQGTLVT |
| | | VSR |
| Anti- | 448 | EVQLVQSGAEVKKPGESLKISCKGSGYAFSNFWMNWVRQ |
| MUC1 | | MPGKGLEWMGQIYPGDGDTNYNGKFKGQVTISADKSISTA |
| VH5 | | YLQWSSLKASDTAMYYCARSYYRSAWFAYWGQGTLVTV |
| | | SL |
| Anti- | 449 | EIVLTQSPDFQSVTPKEKVTITCRASQSIGTSIHWYQQKPDQ |
| MUC1 | | SPKLLIKYASESISGVPSRFSGSGSGTDFTLTINSLEAEDAAT |
| VL1 | | YYCQQSNNWPLTFGQGTKVEIK |
| Anti- | 450 | EIVMTQSPATLSVSPGERATLSCRASQSIGTSIHWYQQKPG |
| MUC1 | | QAPRLLIYYASESISGIPARFSGSGSGTEFTLTISSLQSEDFAV |
| VL2 | | YYCQQSNNWPLTFGGGTKVEIK |
| Anti- | 451 | EIVLTQSPATLSLSPGERATLSCRASQSIGTSIHWYQQKPGQ |
| MUC1 | | APRLLIYYASESISGIPARFSGSGSGTDFTLTISSLEPEDFAVY |
| VL3 | | YCQQSNNWPLTFGGGTKVEIK |
| Anti- | 452 | AIQLTQSPSSLSASVGDRVTITCRASQSIGTSIHWYQQKPGK |
| MUC1 | | APKLLIYYASESISGVPSRFSGSGSGTDFTLTISSLQPEDFAT |
| VL4 | | YYCQQSNNWPLTFGGGTKVEIK |
| Anti- | 453 | DIVMTQSPSLLSASTGDRVTISCRASQSIGTSIHWYQQKPGK |
| MUC1 | | APELLIYYASESISGVPSRFSGSGSGTDFTLTISCLQSEDFAT |
| VL5 | | YYCQQSNNWPLTFGQGTKVEIK |

### Table 31. Exemplary Anti-MUCI Antibody Sequences

Table 31 provides exemplary anti-MUC1 antibody sequences.

| | SEQ | |
|---|---|---|
| | ID | |
| Name | NO | Amino acid sequence |
| Chimeric | 454 | QVQLQQSGAELVRPGSSVKISCKTSGYAFSNFWMNWVKQ |
| antibody_ | | RPGQGLEWIGQIYPGDGDTNYNGKFKGKATLTADKSSSTA |
| VH- | | YMQLSSLTSEASAVYFCARSYYRSAWFAYWGQGTLVSVS |
| hIgG1C | | AASTKGPSVFPLAPSSKSTSGGTAALGCLVKDYFPEPVTVS |
| H1 | | WNSGALTSGVHTFPAVLQSSGLYSLSSVVTVPSSSLGTQTY |
| Chimeric | 455 | DILLTQSPAILSVSPGERVSFSCRASQSIGTSIHWYQQRINGS |
| antibody_ | | PRLLIKYASESISGIPSRFSGSGSGTDFTLSINSVESEDIADYY |
| VL- | | CQQSNNWPLTFGAGTKLELKRTVAAPSVFIFPPSDEQLKSG |
| hIgKCL: | | TASVVCLLNNFYPREAKVQWKVDNALQ |
| VH1- | 456 | QVQLVQSGAEVKKPGSSVKVSCKASGYAFSNFWMNWVR |
| hIgG1C | | QAPGQGLEWMGQIYPGDGDTNYNGKFKGRVTITADKSTS |
| H: | | TAYMELSSLRSEDTAVYYCARSYYRSAWFAYWGQGTLVT |
| | | VSSASTKGPSV |
| VH2- | 457 | QVQLVQSGAEVKKPGASVKVSCKASGYAFSNFWMNWVR |
| hIgG1C | | QAPGQGLEWMGQIYPGDGDTNYNGKFKGRVTMTRDTSTS |
| H: | | TVYMELSSLRSEDTAVYYCARSYYRSAWFAYWGQGTLVT |
| | | VSSASTKGPSV |
| VH3- | 458 | QVQLVQSGAEVKKPGASVKVSCKASGYAFSNFWMNWVR |
| hIgG1C | | QAPGQGLEWMGQIYPGDGDTNYNGKFKGRVTMTRDTSIS |
| H: | | TAYMELSRLRSDDTAVYYCARSYYRSAWFAYWGQGTLVT |
| | | VSSASTKGPSV |
| VH4- | 459 | QVQLVQSGAEVKKPGATVKISCKVSGYAFSNFWMNWVQ |
| hIgG1C | | QAPGKGLEWMGQIYPGDGDTNYNGKFKGRVTITADTSTD |
| H: | | TAYMELSSLRSEDTAVYYCARSYYRSAWFAYWGQGTLVT |
| | | VSRASTKGPSV |
| VH5- | 460 | EVQLVQSGAEVKKPGESLKISCKGSGYAFSNFWMNWVRQ |
| hIgG1C | | MPGKGLEWMGQIYPGDGDTNYNGKFKGQVTISADKSISTA |
| H: | | YLQWSSLKASDTAMYYCARSYYRSAWFAYWGQGTLVTV |
| | | SLASTKGPSV |
| VL1- | 461 | EIVLTQSPDFQSVTPKEKVTITCRASQSIGTSIHWYQQKPDQ |
| hIgKCL | | SPKLLIKYASESISGVPSRFSGSGSGTDFTLTINSLEAEDAAT |
| | | YYCQQSNWPLTFGQGTKVEIKRTVAAPSVFIFPPSDEQLKS |
| VL2- | 462 | EIVMTQSPATLSVSPGERATLSCRASQSIGTSIHWYQQKPG |
| hIgKCL | | QAPRLLIYYASESISGIPARFSGSGSGTEFTLTISSLQSEDFAV |
| | | YYCQQSNN |
| | | WPLTFGGGTKVEIKRTVAAPSVFIFPPSDEQLKS |
| VL3- | 463 | EIVLTQSPATLSLSPGERATLSCRASQSIGTSIHWYQQKPGQ |
| hIgKCL | | APRLLIYYASESISGIPARFSGSGSGTDFTLTISSLEPEDFAVY |
| | | YCQQSNNWPLTFGGGTKVEIKRTVAAPSVFIFPPSDEQLKS |
| VLA- | 464 | AIQLTQSPSSLSASVGDRVTITCRASQSIGTSIHWYQQKPGK |
| hIgKCL | | APKLLIYYASESISGVPSRFSGSGSGTDFTLTISSLQPEDFAT |
| | | YYCQQSNNWPLTFGGGTKVEIKRTVAAPSVFIFPPSDEQLK |
| | | S |
| VL5- | 465 | DIVMTQSPSLLSASTGDRVTISCRASQSIGTSIHWYQQKPGK |
| hIgKCL | | APELLIYYASESISGVPSRFSGSGSGTDFTLTISCLQSEDFAT |
| | | YYCQQSNNWPLTFGQGTKVEIKRTVAAPSVFIFPPSDEQLK |
| | | S |

### Table 32. Exemplary Anti-MUCI-TGFβRII ECD Heavy Chain Sequences

Table 32 provides exemplary anti-MUCI-TGFβRII ECD heavy chain sequences. Such heavy chain polypeptides can be paired with an anti-MUC1 light chain, e.g., SEQ ID NO: 467, to make an anti-MUC1 fusion protein. Such heavy chain polypeptides can be paired with an anti-MUC1 variable region, e.g., SEQ ID NOs.: 449-453, to make an anti-MUC1 fusion protein.

| | SEQ | |
|---|---|---|
| | ID | |
| Name | NO | Amino acid sequence |
| Anti- | 466 | QVQLVQSGAEVKKPGSSVKVSCKASGYAFSNFWMNWVR |
| MUC1 | | QAPGQGLEWMGQIYPGDGDTNYNGKFKGRVTITADKSTS |
| VHI- | | TAYMELSSLRSEDTAVYYCARSYYRSAWFAYWGQGTLVT |
| IgG1(wt)- | | VSSASTKGPSVFPLAPSSKSTSGGTAALGCLVKDYFPEPVT |
| linker- | | VSWNSGALTSGVHTFPAVLQSSGLYSLSSVVTVPSSSLGTQ |
| TGFβRII | | TYICNVNHKPSNTKVDKKVEPKSCDKTHTCPPCPAPELLGG |
| ECD | | PSVFLFPPKPKDTLMISRTPEVTCVVVDVSHEDPEVKFNWY |
| | | VDGVEVHNAKTKPREEQYNSTYRVVSVLTVLHQDWLNGK |
| | | EYKCKVSNKALPAPIEKTISKAKGQPREPQVYTLPPSREEM |
| | | TKNQVSLTCLVKGFYPSDIAVEWESNGQPENNYKTTPPVL |
| | | DSDGSFFLYSKLTVDKSRWQQGNVFSCSVMHEALHNHYT |
| | | QKSLSLSPGKGGGGSGGGGSIPPHVQKSVNNDMIVTDNNG |
| | | AVKFPQLCKFCDVRFSTCDNQKSCMSNCSITSICEKPQEVC |
| | | VAVWRKNDENITLETVCHDPKLPYHDFILEDAASPKCIMK |
| | | EKKKPGETFFMCSCSSDECNDNIIFSEEYNTSNPD |

### Table 33. Exemplary Anti-MUCI Light Chain Sequences

Table 33 provides exemplary anti-MUC1 light chain sequences. Such polypeptides can be paired with an anti-MUC1 heavy chain, e.g., SEQ ID NO: 467, to make an anti-MUC1 fusion protein. Such polypeptides can be paired with an anti-MUC1 variable heavy region, e.g., SEQ ID NOs.: 444-448, to make an anti-MUCI fusion protein.

| | SEQ | |
|---|---|---|
| | ID | |
| Name | NO | Amino acid sequence |
| Anti- | 467 | EIVLTQSPDFQSVTPKEKVTITCRASQSIGTSIHWYQQKPDQ |
| MUC1 | | SPKLLIKYASESISGVPSRFSGSGSGTDFTLTINSLEAEDAAT |
| VL1- | | YYCQQSNNWPLTFGQGTKVEIKTVAAPSVFIFPPSDEQLKS |
| κCL | | GTASVVCLLNNFYPREAKVQWKVDNALQSGNSQESVTEQ |
| | | DSKDSTYSLSSTLTLSKADYEKHKVYACEVTHQGLSSPVT |
| | | KSFNRGEC |

### Table 34. Exemplary TGFβ1 Inhibitory Peptide Sequences

Table 34 provides exemplary TGFβ1 inhibitory peptides for use in MUCI and MUC16 fusion proteins described herein. In any embodiments exemplifying a fusion protein with. e.g., TGFβRII ECD, a TGFβ1 inhibitory peptide may be used instead.

| | SEQ | | SEQ | |
|---|---|---|---|---|
| | ID | Amino Acid | ID | |
| Name | NO | Sequence | NO | Nucleotide Sequence |
| TGFβ1- | 468 | RIWFIPRSSWYE | 508 | AGGATCTGGTTCATCCCCAGGAGCAG |
| I1 | | RA | | CTGGTACGAGAGGGCC |
| TGFβ1- | 469 | IWFIPRSSWYER | 509 | ATCTGGTTCATCCCCAGGAGCAGCTG |
| I2 | | A | | GTACGAGAGGGCC |
| TGFβ1- | 470 | FIPRSSWYERA | 510 | TTCATCCCCAGGAGCAGCTGGTACGA |
| I3 | | | | GAGGGCC |
| TGFβ1- | 471 | FIPRSSWYERA | 511 | TTCATCCCCAGGAGCAGCTGGTACGA |
| I4 | | | | GAGGGCC |
| TGFβ1- | 472 | IPRSSWYERA | 512 | ATCCCCAGGAGCAGCTGGTACGAGAG |
| I5 | | | | GGCC |
| TGFβ1- | 473 | PRSSWYERA | 513 | CCCAGGAGCAGCTGGTACGAGAGGGC |
| I6 | | | | C |
| TGFβ1- | 474 | KRIWFIPRSSWY | 514 | AAGAGGATCTGGTTCATCCCCAGGAG |
| I7 | | ER | | CAGCTGGTACGAGAGG |
| TGFβ1- | 475 | KRIWFIPRSSWY | 515 | AAGAGGATCTGGTTCATCCCCAGGAG |
| I8 | | E | | CAGCTGGTACGAG |
| TGFβ1- | 476 | KRIWFIPRSSWY | 516 | AAGAGGATCTGGTTCATCCCCAGGAG |
| I9 | | | | CAGCTGGTAC |
| TGFβ1- | 477 | KRIWFIPRSSW | 517 | AAGAGGATCTGGTTCATCCCCAGGAG |
| I10 | | | | CAGCTGG |
| TGFβ1- | 478 | KRIWFIPRSS | 518 | AAGAGGATCTGGTTCATCCCCAGGAG |
| I11 | | | | CAGC |
| TGFβ1- | 479 | KRIWFIPRS | 519 | AAGAGGATCTGGTTCATCCCCAGGAG |
| I12 | | | | C |
| TGFβ1- | 480 | RIWFIPRSSWYE | 520 | AGGATCTGGTTCATCCCCAGGAGCAG |
| I13 | | R | | CTGGTACGAGAGG |
| TGFβ1- | 481 | IWFIPRSSWYE | 521 | ATCTGGTTCATCCCCAGGAGCAGCTG |
| I14 | | | | GTACGAG |
| TGFβ1- | 482 | WFIPRSSWY | 522 | TGGTTCATCCCCAGGAGCAGCTGGTA |
| I15 | | | | C |
| TGFβ1- | 483 | DRRIFWWSLRS | 523 | GACAGGAGGATCTTCTGGTGGAGCCT |
| I16 | | APGA | | GAGGAGCGCCCCCGGCGCC |
| TGFβ1- | 484 | HVRLHHYLRHR | 524 | CACGTGAGGCTGCACCACTACCTGAG |
| I17 | | SLPN | | GCACAGGAGCCTGCCCAAC |
| TGFβ1- | 485 | RFFTRFPWHYH | 525 | AGGTTCTTCACCAGGTTCCCCTGGCAC |
| I18 | | ASRL | | TACCACGCCAGCAGGCTG |
| TGFβ1- | 486 | RLAHSHRHRSH | 526 | AGGCTGGCCCACAGCCACAGGCACAG |
| I19 | | V ALT | | GAGCCACGTGGCCCTGACC |
| TGFβ1- | 487 | RRWVRYPVHLH | 527 | AGGAGGTGGGTGAGGTACCCCGTGCA |
| I20 | | SPIV | | CCTGCACAGCCCCATCGTG |
| TGFβ1- | 488 | PPYHRFWRGHR | 528 | CCCCCCTACCACAGGTTCTGGAGGGG |
| I21 | | HAVQ | | CCACAGGCACGCCGTGCAG |
| TGFβ1- | 489 | HRISHFAHRYLA | 529 | CACAGGATCAGCCACTTCGCCCACAG |
| I22 | | RLH | | GTACCTGGCCAGGCTGCAC |
| TGFβ1- | 490 | WHWRHRIPLQL | 530 | TGGCACTGGAGGCACAGGATCCCCCT |
| I23 | | AAGR | | GCAGCTGGCCGCCGGCAGG |
| TGFβ1- | 491 | GWHSLLHSRYH | 531 | GGCTGGCACAGCCTGCTGCACAGCAG |
| I24 | | RIAA | | GTA CCACAGGATCGCCGCC |
| TGFβ1- | 492 | FVWVRFHRLPR | 532 | TTCGTGTGGGTGAGGTTCCACAGGCT |
| I25 | | QIYT | | GCCCAGGCAGATCTACACC |
| TGFβ1- | 493 | WHKYFLRRPLS | 533 | TGGCACAAGTACTTCCTGAGGAGGCC |
| I26 | | VRTR | | CCTGAGCGTGAGGACCAGG |
| TGFβ1- | 494 | VPMALNHGVY | 534 | GTGCCCATGGCCCTGAACCACGGCGT |
| I27 | | VMVSS | | GTACGTGATGGTGAGCAGC |
| TGFβ1- | 495 | RKWFLQHRRMP | 535 | AGGAAGTGGTTCCTGCAGCACAGGAG |
| I28 | | VSVL | | GATGCCCGTGAGCGTGCTG |
| TGFβ1- | 496 | SGRRHLHRHHIF | 536 | AGCGGCAGGAGGCACCTGCACAGGCA |
| I29 | | SLP | | CCACATCTTCAGCCTGCCC |
| TGFβ1- | 497 | GWITFHRRHHD | 537 | GGCTGGATCACCTTCCACAGGAGGCA |
| I30 | | RVLS | | CCACGACAGGGTGCTGAGC |
| TGFβ1- | 498 | RLHGHRSHRFT | 538 | AGGCTGCACGGCCACAGGAGCCACAG |
| I31 | | HVAQ | | GTTCACCCACGTGGCCCAG |
| TGFβ1- | 499 | KRIWFIPRSSWY | 539 | AAGAGGATCTGGTTCATCCCCAGGAG |
| I32 | | ERA | | CAGCTGGTACGAGAGGGCC |
| TGFβ1- | 500 | MPLSRYWWLFS | 540 | ATGCCCCTGAGCAGGTACTGGTGGCT |
| I33 | | HRPR | | GTTCAGCCACAGGCCCAGG |
| TGFβ1- | 501 | RHLSHFKWLRS | 541 | AGGCACCTGAGCCACTTCAAGTGGCT |
| I34 | | HGLD | | GAGGAGCCACGGCCTGGAC |
| TGFβ1- | 502 | RRFHFHSRMVA | 542 | AGGAGGTTCCACTTCCACAGCAGGAT |
| I35 | | VDNS | | GGTGGCCGTGGACAACAGC |
| TGFβ1- | 503 | HANFCLGPCPYI | 543 | CACGCCAACTTCTGCCTGGGCCCCTGC |
| I36 | | WSLA | | CCCTACATCTGGAGCCTGGCC |
| TGFβ1- | 504 | FCLGPCPYIWSL | 544 | TTCTGCCTGGGCCCCTGCCCCTACATC |
| I37 | | DTA | | TGGAGCCTGGACACCGCC |
| TGFβ1- | 505 | SNPYSAFQVDII | 545 | AGCAACCCCTACAGCGCCTTCCAGGT |
| I38 | | VDIA | | GGACATCATCGTGGACATCGCC |
| TGFβ1- | 506 | TSLDATMIWTM | 546 | ACCAGCCTGGACGCCACCATGATCTG |
| I39 | | MA | | GACCATGATGGCC |
| TGFβ1- | 507 | TSLDASIWAMM | 547 | ACCAGCCTGGACGCCAGCATCTGGGC |
| I40 | | QNA | | CATGATGCAGAACGCC |

### Table 35. Exemplary IgG1 Sequences

Table 35 provides exemplary algG1 sequences for use in fusion proteins describe herein. Variants comprising amino acid substitutions and/or deletions may also be utilized.

| | SEQ | |
|---|---|---|
| | ID | |
| Name | NO | Amino acid sequence |
| CH1 | 548 | ASTKGPSVFPLAPSSKSTSGGTAALGCLVKDYFPEPVTVSW |
| (WT) | | NSGALTSGVHTFPAVLQSSGLYSLSSVVTVPSSSLGTQTYIC |
| | | NVNHKPSNTKVDKKV |
| Hinge | 549 | EPKSCDKTHTCP |
| (WT) | | |
| CH2 | 550 | PCPAPELLGGPSVFLFPPKPKDTLMISRTPEVTCVVVDVSHE |
| (WT) | | DPEVKFNWYVDGVEVHNAKTKPREEQYNSTYRVVSVLTV |
| | | LHQDWLNGKEYKCKVSNKALPAPIEKTISKAK |
| CH3 | 551 | GQPREPQVYTLPPSRDELTKNQVSLTCLVKGFYPSDIAVEW |
| (WT) | | ESNGQPENNYKTTPPVLDSDGSFFLYSKLTVDKSRWQQGN |
| | | VFSCSVMHEALHNHYTQKSLSLSPGK |
| CH1 | 552 | ASTKGPSVFPLAPSSKSTSGGTAALGCLVKDYFPEPVTVSW |
| (Variant | | NSGALTSGVHTFPAVLQSSGLYSLSSVVTVPSSSLGTQTYIC |
| 1) | | NVNHKPSNTKVDKRV |
| CH3 | 553 | GQPREPQVYTLPPSREELTKNQVSLTCLVKGFYPSDIAVEW |
| (Variant | | ESNGQPENNYKTTPPVLDSDGSFFLYSKLTVDKSRWQQGN |
| 1) | | VFSCSVMHEALHNHYTQKSLSLSPGK |
| CH3 | 554 | GQPREPQVYTLPPSREEMTKNQVSLTCLVKGFYPSDIAVEW |
| (Variant | | ESNGQPENNYKTTPPVLDSDGSFFLYSKLTVDKSRWQQGN |
| 2) | | VFSCSVMHEALHNHYTQKSLSLSPGK |

1. A method of treating cancer, the method comprising administering to a patient in need thereof either:
(a) a fusion protein comprising:
a first component that is an antibody, or a functional fragment or variant thereof; and
a second component that is either: a cytokine trap, or a functional fragment or variant thereof; or an adenosine deaminase (ADA), or a functional fragment or variant thereof;
or
(b) a polynucleotide encoding the same.

2-13. (canceled)

14. The method of claim 1, wherein the first component of the fusion protein binds to a tumor antigen expressed on the surface of a tumor cell, wherein the tumor antigen is selected from CD19, BCMA, CD23, BAFF-R, GPRC5D, CD44, CAIX, CD5, CD30, CD70, CD44v6, CD44v7, CD44v8, CD174, CD28, CD128, CD138, CS1, CLL-1, L1-CAM, FAP, ROR1, CEA, EGP-2, EGP-40, HER2, HER3, Folate-binding Protein, GD2, GD3, IL-13R-a2, IL-11Ra, EphA2, CSPG4, KDR, EDB-F, mesothelin, CD22, EGFR, Folate receptor «, MUC-1, MUC-4, MUC-16, MAGE-A1, h5T4, PSMA, PSCA, GPC3, c-met, TAG-72, EGFR, CD20, EGFRVIII, CD123, and VEGF-R2.

15-20. (canceled)

21. The method of claim 1, wherein the first component of the fusion protein comprises:
(a) a VL-region comprising a polypeptide that is at least 80% identical to any one of SEQ ID NOs: 417-428; and
(b) a VH-region comprising a polypeptide that is at least 80% identical to any one of SEQ ID NOs: 390-403.

22. (canceled)

23. The method of claim 1, wherein:
(a) the first component of the fusion protein comprises: a VL-region comprising a polypeptide that is at least 80% identical to SEQ ID NO: 426; and a VH region comprising a polypeptide that is at least 80% identical to SEQ ID NO: 399; and
(b) the second component of the fusion protein is a cytokine trap comprising a sequence that is at least 80% identical to SEQ ID NO: 14.

24-29. (canceled)

30. The method of claim 1, wherein the first component of the fusion protein comprises:
(a) a VL-region comprising a polypeptide that is at least 80% identical to any one of SEQ ID NOs: 449-453; and
(b) a VH-region comprising a polypeptide that is at least 80% identical to any one of SEQ ID NOs: 444-448.

31-62. (canceled)

63. The method of claim 1, wherein the cytokine trap is a TGF-β cytokine trap comprising either:
(a) a transforming growth factor beta receptor II (TGFβRII);
(b) a TGFβR extracellular domain;
(c) a TGFβRII extracellular domain;
(d) an anti-TGF-β antibody;
(e) a TGF-β inhibitory peptide;
(f) a TGF-β antagonistic peptide: or
(g) a functional fragment or variant of any of (a) thru (f).

64-68. (canceled)

69. The method of claim 1, wherein the cytokine trap comprises a polypeptide that is at least 80% identical to any one of SEQ ID NOs: 14, 141, 142.

70. The method of claim 1, wherein the cytokine trap is a TGF-β cytokine trap comprising a polypeptide that is at least 80% identical to SEQ ID NO: 14.

71-74. (canceled)

75. The method of claim 1, wherein the cytokine trap is a TGF-β cytokine trap comprising an anti-TGF-β antibody, or an antigen-binding fragment or variant thereof, wherein the anti-TGF-β antibody comprises:
(a) a VH region comprising a polypeptide that is at least 80% identical to any one of SEQ ID NOs: 166, 168, 169, 171, 173, and 175; and/or
(b) a VL region comprising a polypeptide that is at least 80% identical to any one of SEQ ID NOs: 165, 167, 170, 172, 174, 176, and 178.

76-83. (canceled)

84. The method of claim 1, wherein the second component of the fusion protein is an ADA, or a functional fragment or variant thereof.

85-86. (canceled)

87. The method of claim 84, wherein the ADA comprises a polypeptide that is at least 80% identical to any one of SEQ ID NOs: 273-279 and 284.

88. The method of claim 1, wherein the first and second components of the fusion protein are connected by a linker comprising:
(a) (G4S)ₙ, wherein n is 2, 3, 4, 5, or 6 (SEQ ID NO: 555);
(b) (Gly)ₙ wherein n is 6, 7, or 8 (SEO ID NO: 33);
(c) (EAAAK)ₙ, wherein n is 1, 2, 3, 4, 5, or 6 (SEQ ID NO: 34);
(d) A (EAAAK)₄ALEA (EAAAK)₄A (SEO ID NO: 31); and/or
(e) a sequence of any one of SEO ID NOs: 17-34.

89-111. (canceled)

112. The method of claim 1, further comprising administering to the patient a fusion protein comprising IL-15 and IL-15Rα.

113-118. (canceled)

119. The method of claim 1, further comprising administering to the patient an effective amount of T cells engineered to express an exogenous receptor.

120-124. (canceled)

125. The method of claim 1, wherein the effective amount of engineered T-cells is at least 10² cells/kg.

126-130. (canceled)

131. The method of claim 1, wherein the first component of the fusion protein comprises:
(a) a VL region comprising the sequence of any one of SEQ ID NOs: 423, 425, and 426; and
(b) a VH region comprising the sequence of SEQ ID NO: 399.

132. The method of claim 131, wherein the first component of the fusion protein comprises a VL region comprising the sequence of any one of SEQ ID NO: 423.

133. The method of claim 131, wherein the first component of the fusion protein comprises a VL region comprising the sequence of any one of SEQ ID NO: 425.

134. The method of claim 131, wherein the first component of the fusion protein comprises a VL region comprising the sequence of any one of SEQ ID NO: 426.

135. The method of claim 131, wherein:
(a) the first component of the fusion protein comprises:
(i) a VL region comprising the sequence of SEQ ID NO: 426; and
(ii) a VH region comprising the sequence of SEQ ID NO: 399; and
(b) the second component of the fusion protein is a cytokine trap comprising the sequence of SEQ ID NO: 14.


SEQUENCE LISTING (564 sequences)

SEQ ID NO: 1 - protein, 113 aa, synthetic construct
  REGION 1..113: note = Description of Artificial Sequence: Synthetic polypeptide
QVQLVESGGGVVQPGRSLRLSCAASGFTFRSYGMHWVRQAPGKGLEWVAIIFYDGSNKYYADSVKGRFTISRDNSKNTLYLQMNSLRAEDTAVYYCARDDDYWGQGALVTVSS

SEQ ID NO: 2 - protein, 114 aa, synthetic construct
  REGION 1..114: note = Description of Artificial Sequence: Synthetic polypeptide
EVQLVQSGGGVVQPGRSLRLSCAASGFTFSSYGMHWVRQAPGKGLEWVAVIWYDGSNKYYADSVKGRFTISRDNSKNTLYLQMNSLRAEDTAVYYCAGEGFDYWGQGTLVTVSS

SEQ ID NO: 3 - protein, 120 aa, synthetic construct
  REGION 1..120: note = Description of Artificial Sequence: Synthetic polypeptide
QVQLVQSGAEVKKPGASVKVSCKASGYTFTGYYMHWVRQAPGQGLEWMGAINPNSGGTNYAQKFQGRVTMTRDTSISTAYMELSRLRSDDTAVYYCARRPDRANWHFDYWGQGTLVTVSS

SEQ ID NO: 4 - protein, 120 aa, synthetic construct
  REGION 1..120: note = Description of Artificial Sequence: Synthetic polypeptide
QVQLVQSGAEVKKPGASVKVSCKASGYTFTGYYMHWVRQAPGQGLEWMGAINPNSGGTNYAQKLQGRVTMTTDTSTSTAYMELRSLRSDDTAVYYCARHGLKGDGYFDYWGQGTLVTVSS

SEQ ID NO: 5 - protein, 113 aa, synthetic construct
  REGION 1..113: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNNDYWGQGTLVTVSS

SEQ ID NO: 6 - protein, 113 aa, synthetic construct [sequence omitted]

SEQ ID NO: 7 - protein, 120 aa, synthetic construct [sequence omitted]

SEQ ID NO: 8 - protein, 107 aa, synthetic construct
  REGION 1..107: note = Description of Artificial Sequence: Synthetic polypeptide
EIVMTQSPATLSLSPGERATLSCRASQSVSSYLAWYQQKPGQAPRLLIYDASNRATGIPARFSGSGSGTDFTLTISSLEPEDFAVYYCQQYNNWPRTFGQGTKVEIK

SEQ ID NO: 9 - protein, 107 aa, synthetic construct
  REGION 1..107: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPKTFGQGTKVEIK

SEQ ID NO: 10 - protein, 114 aa, synthetic construct
  REGION 1..114: note = Description of Artificial Sequence: Synthetic polypeptide
RNVLTQSPLSLPVTPGEPASISCRSSQSLSSSGYTYLDWYLQKPGQSPQLLIYLASWRDSGVPDRFSGSGSGTDFTLKISRVEAEDVGVYYCMQAEQTPGPGNTFGQGTKLEIK

SEQ ID NO: 11 - protein, 112 aa, synthetic construct
  REGION 1..112: note = Description of Artificial Sequence: Synthetic polypeptide
DVVMTQSPLSLPVTPGEPASISCRSSQSLLHTNGYNYLHWYLQKPGQSPQLLIYLGSWQDSGVPDRFSGSGSGTDFTLKISRVEAEDVGVYYCMQAEQTPRTFGQGTRLEVK

SEQ ID NO: 12 - protein, 107 aa, synthetic construct [sequence omitted]

SEQ ID NO: 13 - protein, 111 aa, synthetic construct [sequence omitted]

SEQ ID NO: 14 - protein, 136 aa, synthetic construct
  REGION 1..136: note = Description of Artificial Sequence: Synthetic polypeptide
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 15 - protein, 214 aa, synthetic construct
  REGION 1..214: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 586 aa, synthetic construct
  REGION 1..586: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 17 - protein, 16 aa, synthetic construct
  REGION 1..16: note = Description of Artificial Sequence: Synthetic peptide
DPGGGGSGGGGSNPGS

SEQ ID NO: 18 - protein, 16 aa, synthetic construct
  REGION 1..16: note = Description of Artificial Sequence: Synthetic peptide
GGGGSGGGGSGSDPGS

SEQ ID NO: 19 - protein, 16 aa, synthetic construct
  REGION 1..16: note = Description of Artificial Sequence: Synthetic peptide
DPGSGGGGSGGGGSGS

SEQ ID NO: 20 - protein, 19 aa, synthetic construct
  REGION 1..19: note = Description of Artificial Sequence: Synthetic peptide
GGGGSGGGGSGGGGSDPGS

SEQ ID NO: 21 - protein, 19 aa, synthetic construct
  REGION 1..19: note = Description of Artificial Sequence: Synthetic peptide
DPGSGGGGSGGGGSGGGGS

SEQ ID NO: 22 - protein, 17 aa, synthetic construct
  REGION 1..17: note = Description of Artificial Sequence: Synthetic peptide
DPGSGSVPLGSGSNPGS

SEQ ID NO: 23 - protein, 19 aa, synthetic construct
  REGION 1..19: note = Description of Artificial Sequence: Synthetic peptide
DPGSGGSVPLGSGGSNPGS

SEQ ID NO: 24 - protein, 20 aa, synthetic construct
  REGION 1..20: note = Description of Artificial Sequence: Synthetic peptide
DPGVLEREDKPTTSKPNPGS

SEQ ID NO: 25 - protein, 20 aa, synthetic construct
  REGION 1..20: note = Description of Artificial Sequence: Synthetic peptide
DPGVLEREDVPTTSYPNPGS

SEQ ID NO: 26 - protein, 20 aa, synthetic construct
  REGION 1..20: note = Description of Artificial Sequence: Synthetic peptide
DPGVLEREDKVTTSKYNPGS

SEQ ID NO: 27 - protein, 18 aa, synthetic construct
  REGION 1..18: note = Description of Artificial Sequence: Synthetic peptide
DPVLEREDKVTTSKNPGS

SEQ ID NO: 28 - protein, 7 aa, synthetic construct
  REGION 1..7: note = Description of Artificial Sequence: Synthetic peptide
DIEGRMD

SEQ ID NO: 29 - protein, 15 aa, synthetic construct
  REGION 1..15: note = Description of Artificial Sequence: Synthetic peptide
GEGKSSGSGSESKAS

SEQ ID NO: 30 - protein, 18 aa, synthetic construct
  REGION 1..18: note = Description of Artificial Sequence: Synthetic peptide
GSTSGSGKPGSGEGSTKG

SEQ ID NO: 31 - protein, 46 aa, synthetic construct
  REGION 1..46: note = Description of Artificial Sequence: Synthetic polypeptide
AEAAAKEAAAKEAAAKEAAAKALEAEAAAKEAAAKEAAAKEAAAKA

SEQ ID NO: 32 - protein, 50 aa, synthetic construct
  REGION 1..50: note = Description of Artificial Sequence: Synthetic polypeptide
  VARIANT 1..50: note = This sequence may encompass 1-10 "Gly Gly Gly Gly Ser" repeating units
GGGGSGGGGSGGGGSGGGGSGGGGSGGGGSGGGGSGGGGSGGGGSGGGGS

SEQ ID NO: 33 - protein, 8 aa, synthetic construct
  REGION 1..8: note = Description of Artificial Sequence: Synthetic peptide
  VARIANT 1..8: note = This sequence may encompass 6-8 residues
GGGGGGGG

SEQ ID NO: 34 - protein, 30 aa, synthetic construct
  REGION 1..30: note = Description of Artificial Sequence: Synthetic polypeptide
  VARIANT 1..30: note = This sequence may encompass 1-6 "Glu Ala Ala Ala Lys" repeating units
EAAAKEAAAKEAAAKEAAAKEAAAKEAAAK

SEQ ID NO: 35 - protein, 111 aa, synthetic construct
  REGION 1..111: note = Description of Artificial Sequence: Synthetic polypeptide
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 36 - protein, 116 aa, synthetic construct
  REGION 1..116: note = Description of Artificial Sequence: Synthetic polypeptide
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 37 - protein, 117 aa, synthetic construct
  REGION 1..117: note = Description of Artificial Sequence: Synthetic polypeptide
QVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSS

SEQ ID NO: 38 - protein, 108 aa, synthetic construct
  REGION 1..108: note = Description of Artificial Sequence: Synthetic polypeptide
DIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKR

SEQ ID NO: 39 - protein, 120 aa, synthetic construct
  REGION 1..120: note = Description of Artificial Sequence: Synthetic polypeptide
EVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSS

SEQ ID NO: 40 - protein, 115 aa, synthetic construct
  REGION 1..115: note = Description of Artificial Sequence: Synthetic polypeptide
DIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVS

SEQ ID NO: 41 - protein, 118 aa, synthetic construct
  REGION 1..118: note = Description of Artificial Sequence: Synthetic polypeptide
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 42 - protein, 113 aa, synthetic construct
  REGION 1..113: note = Description of Artificial Sequence: Synthetic polypeptide
NIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKR

SEQ ID NO: 43 - protein, 113 aa, synthetic construct
  REGION 1..113: note = Description of Artificial Sequence: Synthetic polypeptide
DIELTQSPSSLAVSAGEKVTMSCKSSQSLLNSRTRKNQLAWYQQKPGQSPELLIYWASTRQSGVPDRFTGSGSGTDFTLTISSVQAEDLAVYYCQQSYNLLTFGPGTKLEVKR

SEQ ID NO: 44 - protein, 122 aa, synthetic construct
  REGION 1..122: note = Description of Artificial Sequence: Synthetic polypeptide
VKLQESGGGFVKPGGSLKVSCAASGFTFSSYAMSWVRLSPEMRLEWVATISSAGGYIFYSDSVQGRFTISRDNAKNTLHLQMGSLRSGDTAMYYCARQGFGNYGDYYAMDYWGQGTTVTVSS

SEQ ID NO: 45 - protein, 113 aa, synthetic construct
  REGION 1..113: note = Description of Artificial Sequence: Synthetic polypeptide
DIELTQSPSSLAVSAGEKVTMSCKSSQSLLNSRTRKNQLAWYQQKTGQSPELLIYWASTRQSGVPDRFTGSGSGTDFTLTISSVQAEDLAVYYCQQSYNLLTFGPGTKLEIKR

SEQ ID NO: 46 - protein, 121 aa, synthetic construct
  REGION 1..121: note = Description of Artificial Sequence: Synthetic polypeptide
VKLEESGGGFVKPGGSLKISCAASGFTFRNYAMSWVRLSPEMRLEWVATISSAGGYIFYSDSVQGRFTISRDNAKNTHLQMGSLRSGDTAMYYCARQGFGNYGDYYAMDYWGQGTTVTVSS

SEQ ID NO: 47 - protein, 106 aa, synthetic construct
  REGION 1..106: note = Description of Artificial Sequence: Synthetic polypeptide
DIKMAQSPSSVNASLGERVTITCKASRDINNFLSWFHQKPGKSPKTLIYRANRLVDGVPSRFSGSGSGQDYSFTISSLEYEDVGIYYCLQYGDLYTFGGGTKLEIK

SEQ ID NO: 48 - protein, 113 aa, synthetic construct
  REGION 1..113: note = Description of Artificial Sequence: Synthetic polypeptide
DVQLLESGPGLVRPSQSLSLTCSVTGYSIVSHYYWNWIRQFPGNKLEWMGYISSDGSNEYNPSLKNRISISLDTSKNQFFLKFDFVTTADTATYFCVRGVDYWGQGTTLTVSS

SEQ ID NO: 49 - protein, 107 aa, synthetic construct
  REGION 1..107: note = Description of Artificial Sequence: Synthetic polypeptide
DIQMTQSSSFLSVSLGGRVTITCKASDLIHNWLAWYQQKPGNAPRLLISGATSLETGVPSRFSGSGSGNDYTLSIASLQTEDAATYYCQQYWTTPFTFGSGTKLEIK

SEQ ID NO: 50 - protein, 116 aa, synthetic construct
  REGION 1..116: note = Description of Artificial Sequence: Synthetic polypeptide
DVQLQESGPGLVNPSQSLSLTCTVTGYSITNDYAWNWIRQFPGNKLEWMGYINYSGYTTYNPSLKSRISITRDTSKNQFFLHLNSVTTEDTATYYCARWDGGLTYWGQGTLVTVSA

SEQ ID NO: 51 - protein, 108 aa, synthetic construct
  REGION 1..108: note = Description of Artificial Sequence: Synthetic polypeptide
DIQMTQSPSSLSASVGDRVTITCKASDLIHNWLAWYQQKPGKAPKLLISGATSLETGVPSRFSGSGSGTDFTLTISSLQPEDFATYYCQQYWTTPFTFGQGTKVEIKR

SEQ ID NO: 52 - protein, 116 aa, synthetic construct
  REGION 1..116: note = Description of Artificial Sequence: Synthetic polypeptide
EVQLVESGGGLVQPGGSLRLSCAASGYSITNDYAWNWVRQAPGKGLEWVGYINYSGYTTYNPSLKSRFTISRDNSKNTLYLQMNSLRAEDTAVYYCARWDGGLTYWGQGTLVTVSS

SEQ ID NO: 53 - protein, 116 aa, synthetic construct
  REGION 1..116: note = Description of Artificial Sequence: Synthetic polypeptide
EVQLVESGGGLVQPGGSLRLSCAASGYSITNDYAWNWVRQAPGKGLEWVGYINYSGYTTYNPSLKSRFTISRDNSKNTFYLQMNSLRAEDTAVYYCARWDGGLTYWGQGTLVTVSS

SEQ ID NO: 54 - protein, 109 aa, synthetic construct
  REGION 1..109: note = Description of Artificial Sequence: Synthetic polypeptide
DIVLTQSPAIMSASLGERVTMTCTASSSVSSSYLHWYQQKPGSSPKLWIYSTSNLASGVPGRFSGSGSGTSYSLTISSMEAEDAATYYCHQYHRSPYTFGGGTKVEIKR

SEQ ID NO: 55 - protein, 109 aa, synthetic construct
  REGION 1..109: note = Description of Artificial Sequence: Synthetic polypeptide
DIQMTQSPSSLSASVGDRVTITCTASSSVSSSYLHWYQQKPGKAPKLLIYSTSNLASGVPSRFSGSGSGTDFTLTISSLQPEDFATYYCHQYHRSPYTFGQGTKVEIKR

SEQ ID NO: 56 - protein, 120 aa, synthetic construct
  REGION 1..120: note = Description of Artificial Sequence: Synthetic polypeptide
EVQLVESGGGLVQPGGSLRLSCAASGFNIKDTYMHWVRQAPGKGLEWVGRVDPANGNTKYDPKFQGRFTISADTSKNTAYLQMNSLRAEDTAVYYCVRDYYGHTYGFAFWGQGTLVTVSS

SEQ ID NO: 57 - protein, 47 aa, synthetic construct
  REGION 1..47: note = Description of Artificial Sequence: Synthetic polypeptide
KPTTTPAPRPPTPAPTIASQPLSLRPEACRPAAGGAVHTRGLDFACD

SEQ ID NO: 58 - protein, 94 aa, synthetic construct
  REGION 1..94: note = Description of Artificial Sequence: Synthetic polypeptide
KPTTTPAPRPPTPAPTIASQPLSLRPEASRPAAGGAVHTRGLDFASDKPTTTPAPRPPTPAPTIASQPLSLRPEACRPAAGGAVHTRGLDFACD

SEQ ID NO: 59 - protein, 141 aa, synthetic construct
  REGION 1..141: note = Description of Artificial Sequence: Synthetic polypeptide
KPTTTPAPRPPTPAPTIASQPLSLRPEASRPAAGGAVHTRGLDFASDKPTTTPAPRPPTPAPTIASQPLSLRPEASRPAAGGAVHTRGLDFASDKPTTTPAPRPPTPAPTIASQPLSLRPEACRPAAGGAVHTRGLDFACD

SEQ ID NO: 60 - protein, 185 aa, synthetic construct
  REGION 1..185: note = Description of Artificial Sequence: Synthetic polypeptide
TTPAPRPPTPAPTIASQPLSLRPEASRPAAGGAVHTRGLDFASDKPTTTPAPRPPTPAPTIASQPLSLRPEASRPAAGGAVHTRGLDFASDKPTTTPAPRPPTPAPTIASQPLSLRPEASRPAAGGAVHTRGLDFASDKPTTTPAPRPPTPAPTIASQPLSLRPEACRPAAGGAVHTRGLDFACD

SEQ ID NO: 61 - protein, 28 aa, synthetic construct
  REGION 1..28: note = Description of Artificial Sequence: Synthetic peptide
IYIWAPLAGTCGVLLLSLVITLYCNHRN

SEQ ID NO: 62 - protein, 27 aa, synthetic construct
  REGION 1..27: note = Description of Artificial Sequence: Synthetic peptide
FWVLVVVGGVLACYSLLVTVAFIIFWV

SEQ ID NO: 63 - protein, 42 aa, synthetic construct
  REGION 1..42: note = Description of Artificial Sequence: Synthetic polypeptide
KRGRKKLLYIFKQPFMRPVQTTQEEDGCSCRFPEEEEGGCEL

SEQ ID NO: 64 - protein, 41 aa, synthetic construct
  REGION 1..41: note = Description of Artificial Sequence: Synthetic polypeptide
RSKRSRGGHSDYMNMTPRRPGPTRKHYQPYAPPRDFAAYRS

SEQ ID NO: 65 - protein, 24 aa, synthetic construct
  REGION 1..24: note = Description of Artificial Sequence: Synthetic peptide
LCARPRRSPAQEDGKVYINMPGRG

SEQ ID NO: 66 - protein, 52 aa, synthetic construct
  REGION 1..52: note = Description of Artificial Sequence: Synthetic polypeptide
YFLGRLVPRGRGAAEAATRKQRITETESPYQELQGQRSDVYSDLNTQRPYYK

SEQ ID NO: 67 - protein, 112 aa, synthetic construct
  REGION 1..112: note = Description of Artificial Sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 335 aa, synthetic construct
  REGION 1..335: note = Description of Artificial Sequence: Synthetic polypeptide
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 69 - protein, 243 aa, synthetic construct
  REGION 1..243: note = Description of Artificial Sequence: Synthetic polypeptide
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGGGSGGGSGGGGSGGGGSFWVLVVVGGVLACYSLLVTVAFIIFWVRSKRS

SEQ ID NO: 70 - protein, 297 aa, synthetic construct
  REGION 1..297: note = Description of Artificial Sequence: Synthetic polypeptide
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 71 - protein, 263 aa, synthetic construct
  REGION 1..263: note = Description of Artificial Sequence: Synthetic polypeptide
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIE

SEQ ID NO: 72 - DNA, 333 bp, synthetic construct
  misc_feature 1..333: note = Description of Artificial Sequence: Synthetic polynucleotide
  source 1..333: mol_type = other DNA
gacattcagatgacccagtctccgagctctctgtccgcatcagtaggagacagggtcaccatcacatgcagagccagcgaaagtgtcgacaattatggcattagctttatgaactggttccaacagaaacccgggaaggctcctaagcttctgatttacgctgcatccaaccaaggctccggggtaccctctcgcttctcaggcagtggatctgggacagacttcactctcaccatttcatctctgcagcctgatgacttcgcaacctattactgtcagcaaagtaaggaggttccgtggacgttcggtcaagggaccaaggtggagatcaaa

SEQ ID NO: 73 - DNA, 348 bp, synthetic construct
  misc_feature 1..348: note = Description of Artificial Sequence: Synthetic polynucleotide
  source 1..348: mol_type = other DNA
caggttcagctggtgcagtctggagctgaggtgaagaagcctgggagctcagtgaaggtttcctgcaaagcttctggctacaccttcactgactacaacatgcactgggtgaggcaggctcctggccaaggcctggaatggattggatatatttatccttacaatggtggtaccggctacaaccagaagttcaagagcaaggccacaattacagcagacgagagtactaacacagcctacatggaactctccagcctgaggtctgaggacactgcagtctattactgcgcaagagggcgccccgctatggactactggggccaagggactctggtcactgtctcttca

SEQ ID NO: 74 - DNA, 339 bp, synthetic construct
  misc_feature 1..339: note = Description of Artificial Sequence: Synthetic polynucleotide
  source 1..339: mol_type = other DNA
gacatcgagctgacacagagcccatctagcctggctgtgtctgccggcgagaaagtgaccatgagctgcaagagcagccagagcctgctgaacagccggaccagaaagaatcagctggcctggtatcagcagaagcccggccaatctcctgagctgctgatctactgggccagcacaagacagagcggcgtgcccgatagattcacaggatctggcagcggcaccgacttcaccctgacaatcagttctgtgcaggccgaggacctggccgtgtactactgtcagcagagctacaacctgctgaccttcggacccggcaccaagctggaagtgaagaga

SEQ ID NO: 75 - DNA, 366 bp, synthetic construct
  misc_feature 1..366: note = Description of Artificial Sequence: Synthetic polynucleotide
  source 1..366: mol_type = other DNA
gtgaagctgcaagagtccggcggaggctttgtgaagcctggcggctctctgaaagtgtcctgtgccgccagcggcttcacctttagcagctacgccatgagctgggtccgactgagccctgagatgagactggaatgggtcgccaccatcagtagcgcaggcggctacatcttctacagcgactctgtgcagggcagattcaccatcagccgggacaacgccaagaacaccctgcacctccagatgggcagtctgagaagcggcgataccgccatgtactactgcgccagacaaggcttcggcaactacggcgactactatgccatggattactggggccagggcaccaccgtgacagtctcttct

SEQ ID NO: 76 - DNA, 339 bp, synthetic construct
  misc_feature 1..339: note = Description of Artificial Sequence: Synthetic polynucleotide
  source 1..339: mol_type = other DNA
gacatcgagctgacacagagcccatctagcctggctgtgtctgccggcgagaaagtgaccatgagctgcaagagcagccagagcctgctgaacagccggaccagaaagaatcagctggcctggtatcagcagaaaaccggacagagccccgagctgctgatctactgggccagcacaagacagagcggcgtgcccgatagattcacaggatctggcagcggcaccgacttcaccctgacaatcagttctgtgcaggccgaggacctggccgtgtactactgtcagcagagctacaacctgctgaccttcggacccggcaccaagctggaaatcaagaga

SEQ ID NO: 77 - DNA, 366 bp, synthetic construct
  misc_feature 1..366: note = Description of Artificial Sequence: Synthetic polynucleotide
  source 1..366: mol_type = other DNA
gtgaagctggaagagtccggcggaggctttgtgaagcctggcggaagcctgaagatcagctgtgccgccagcggcttcaccttcagaaactacgccatgagctgggtccgactgagccccgagatgagactggaatgggtcgccacaatcagcagcgcaggcggctacatcttctacagcgatagcgtgcagggcagattcaccatcagccgggacaacgccaagaacaccctgcacctccagatgggcagtctgagatctggcgacaccgccatgtactactgcgccagacaaggcttcggcaactacggcgactactatgccatggattactggggccagggcaccaccgtgacagtctcttct

SEQ ID NO: 78 - DNA, 318 bp, synthetic construct
  misc_feature 1..318: note = Description of Artificial Sequence: Synthetic polynucleotide
  source 1..318: mol_type = other DNA
gacatcaagatggctcagtccccttctagcgtgaatgcttcgctaggggagcgtgtgaccatcacatgtaaagcatcacgcgacataaataatttcctttcctggtttcatcagaaaccgggcaagtcgcctaagacgctgatttacagagcaaatcggttggtagatggagtgccaagcagattcagcgggagcggaagtggacaggattatagcttcactatttcatccctggaatacgaggacgtaggtatctattattgcctccagtatggcgatctttacacatttggtggggggactaagctggagattaag

SEQ ID NO: 79 - DNA, 339 bp, synthetic construct
  misc_feature 1..339: note = Description of Artificial Sequence: Synthetic polynucleotide
  source 1..339: mol_type = other DNA
gacgtgcaacttctggagagcgggccagggctagtcaggccctcccagtcgctttcactgacttgcagtgtgaccggttactctattgtgagtcactactattggaactggattcggcagttcccaggcaacaaactggaatggatggggtacatatcttccgatggctcgaatgaatataacccatcattgaaaaatcgtatttccatcagtctggatacgagtaaaaaccagtttttcctcaaattcgatttcgtgactacagcagatactgccacatacttctgtgtacgaggtgtcgattattggggacagggcacaacgctgaccgtaagttct

SEQ ID NO: 80 - DNA, 321 bp, synthetic construct
  misc_feature 1..321: note = Description of Artificial Sequence: Synthetic polynucleotide
  source 1..321: mol_type = other DNA
gacatccagatgacccagagcagcagcttcctgagcgtgtcccttggcggcagagtgaccatcacctgtaaagccagcgacctgatccacaactggctggcctggtatcagcagaagcctggcaacgctcccagactgctgattagcggcgccacctctctggaaacaggcgtgccaagcagattttccggcagcggctccggcaacgactacacactgtctattgccagcctgcagaccgaggatgccgccacctattactgccagcagtactggaccacacctttcacctttggcagcggcaccaagctggaaatcaag

SEQ ID NO: 81 - DNA, 348 bp, synthetic construct
  misc_feature 1..348: note = Description of Artificial Sequence: Synthetic polynucleotide
  source 1..348: mol_type = other DNA
gacgttcagctgcaagagtctggccctggcctggtcaatcctagccagagcctgagcctgacatgtaccgtgaccggctacagcatcaccaacgactacgcctggaactggatcagacagttccccggcaacaagctggaatggatgggctacatcaactacagcggctacaccacctacaatcccagcctgaagtcccggatctccatcaccagagacaccagcaagaaccagttcttcctgcacctgaacagcgtgaccaccgaggataccgccacctactactgcgctagatgggatggcggcctgacatattggggccagggaacactggtcaccgtgtctgct

SEQ ID NO: 82 - DNA, 324 bp, synthetic construct
  misc_feature 1..324: note = Description of Artificial Sequence: Synthetic polynucleotide
  source 1..324: mol_type = other DNA
gacatccagatgacccagagccccagcagcctgagcgccagcgtgggcgacagggtgaccatcacctgcaaggccagcgacctgatccacaactggctggcctggtaccagcagaagcccggcaaggcccccaagctgctgatcagcggcgccaccagcctggagaccggcgtgcccagcaggttcagcggcagcggcagcggcaccgacttcaccctgaccatcagcagcctgcagcccgaggacttcgccacctactactgccagcagtactggaccacccccttcaccttcggccagggcaccaaggtggagatcaagagg

SEQ ID NO: 83 - DNA, 348 bp, synthetic construct
  misc_feature 1..348: note = Description of Artificial Sequence: Synthetic polynucleotide
  source 1..348: mol_type = other DNA
gaggtgcagctggtggagagcggcggcggcctggtgcagcccggcggcagcctgaggctgagctgcgccgccagcggctacagcatcaccaacgactacgcctggaactgggtgaggcaggcccccggcaagggcctggagtgggtgggctacatcaactacagcggctacaccacctacaaccccagcctgaagagcaggttcaccatcagcagggacaacagcaagaacaccctgtacctgcagatgaacagcctgagggccgaggacaccgccgtgtactactgcgccaggtgggacggcggcctgacctactggggccagggcaccctggtgaccgtgagcagc

SEQ ID NO: 84 - DNA, 348 bp, synthetic construct
  misc_feature 1..348: note = Description of Artificial Sequence: Synthetic polynucleotide
  source 1..348: mol_type = other DNA
gaggtgcagctggtggagagcggcggcggcctggtgcagcccggcggcagcctgaggctgagctgcgccgccagcggctacagcatcaccaacgactacgcctggaactgggtgaggcaggcccccggcaagggcctggagtgggtgggctacatcaactacagcggctacaccacctacaaccccagcctgaagagcaggttcaccatcagcagggacaacagcaagaacaccttctacctgcagatgaacagcctgagggccgaggacaccgccgtgtactactgcgccaggtgggacggcggcctgacctactggggccagggcaccctggtgaccgtgagcagc

SEQ ID NO: 85 - DNA, 327 bp, synthetic construct
  misc_feature 1..327: note = Description of Artificial Sequence: Synthetic polynucleotide
  source 1..327: mol_type = other DNA
gacatcgtgctgacacagagccctgccatcatgtctgccagcctcggcgagcgagtgaccatgacatgtacagccagcagcagcgtgtccagcagctacctgcattggtatcagcagaagcccggcagcagccccaagctgtggatctacagcacaagcaatctggccagcggcgtgccaggcagattttctggttctggcagcggcaccagctacagcctgacaatcagcagcatggaagccgaggatgccgccacctactactgccaccagtaccacagaagcccctacacctttggcggaggcaccaaggtggaaatcaagcgg

SEQ ID NO: 86 - DNA, 327 bp, synthetic construct
  misc_feature 1..327: note = Description of Artificial Sequence: Synthetic polynucleotide
  source 1..327: mol_type = other DNA
gacatccagatgacacagagccctagcagcctgtctgccagcgtgggagacagagtgaccatcacctgtacagccagcagcagcgtgtccagcagctacctgcattggtatcagcagaagcccggcaaggcccctaagctgctgatctacagcaccagcaatctggccagcggcgtgccaagcagattttctggctctggcagcggcaccgacttcaccctgaccatatctagcctgcagcctgaggacttcgccacctactactgccaccagtaccacagaagcccctacacctttggccagggcaccaaggtggaaatcaagcgg

SEQ ID NO: 87 - DNA, 360 bp, synthetic construct
  misc_feature 1..360: note = Description of Artificial Sequence: Synthetic polynucleotide
  source 1..360: mol_type = other DNA
gaggtgcagctggttgaatctggcggaggactggttcagcctggcggatctctgagactgtcttgtgccgccagcggcttcaacatcaaggacacctacatgcactgggtccgacaggcccctggcaaaggacttgagtgggttggaagagtggaccccgccaacggcaacaccaaatacgaccccaagttccagggcagattcaccatcagcgccgacaccagcaagaacaccgcctacctgcagatgaacagcctgagagccgaggacaccgccgtgtactattgcgtgcgggattactacggccatacctacggcttcgccttttggggccagggcacactggttaccgttagctct

SEQ ID NO: 88 - DNA, 141 bp, synthetic construct
  misc_feature 1..141: note = Description of Artificial Sequence: Synthetic polynucleotide
  source 1..141: mol_type = other DNA
aagcccaccaccacccctgcccctagacctccaaccccagcccctacaatcgccagccagcccctgagcctgaggcccgaagcctgtagacctgccgctggcggagccgtgcacaccagaggcctggatttcgcctgcgac

SEQ ID NO: 89 - DNA, 282 bp, synthetic construct
  misc_feature 1..282: note = Description of Artificial Sequence: Synthetic polynucleotide
  source 1..282: mol_type = other DNA
aaacctactacaactcctgccccccggcctcctacaccagctcctactatcgcctcccagccactcagtctcagacccgaggcttctaggccagcggccggaggcgcggtccacacccgcgggctggactttgcatccgataagcccaccaccacccctgcccctagacctccaaccccagcccctacaatcgccagccagcccctgagcctgaggcccgaagcctgtagacctgccgctggcggagccgtgcacaccagaggcctggatttcgcctgcgac

SEQ ID NO: 90 - DNA, 423 bp, synthetic construct
  misc_feature 1..423: note = Description of Artificial Sequence: Synthetic polynucleotide
  source 1..423: mol_type = other DNA
aagcctaccaccacccccgcacctcgtcctccaacccctgcacctacgattgccagtcagcctctttcactgcggcctgaggccagcagaccagctgccggcggtgccgtccatacaagaggactggacttcgcgtccgataaacctactaccactccagccccaaggcccccaaccccagcaccgactatcgcatcacagcctttgtcactgcgtcctgaagccagccggccagctgcagggggggccgtccacacaaggggactcgactttgcgagtgataagcccaccaccacccctgcccctagacctccaaccccagcccctacaatcgccagccagcccctgagcctgaggcccgaagcctgtagacctgccgctggcggagccgtgcacaccagaggcctggatttcgcctgcgac

SEQ ID NO: 91 - DNA, 564 bp, synthetic construct
  misc_feature 1..564: note = Description of Artificial Sequence: Synthetic polynucleotide
  source 1..564: mol_type = other DNA
aagcctaccaccacccccgcacctcgtcctccaacccctgcacctacgattgccagtcagcctctttcactgcggcctgaggccagcagaccagctgccggcggtgccgtccatacaagaggactggacttcgcgtccgataaacctactaccactccagccccaaggcccccaaccccagcaccgactatcgcatcacagcctttgtcactgcgtcctgaagccagccggccagctgcagggggggccgtccacacaaggggactcgactttgcgagtgataaacctactacaactcctgccccccggcctcctacaccagctcctactatcgcctcccagccactcagtctcagacccgaggcttctaggccagcggccggaggcgcggtccacacccgcgggctggactttgcatccgataagcccaccaccacccctgcccctagacctccaaccccagcccctacaatcgccagccagcccctgagcctgaggcccgaagcctgtagacctgccgctggcggagccgtgcacaccagaggcctggatttcgcctgcgac

SEQ ID NO: 92 - DNA, 84 bp, synthetic construct
  misc_feature 1..84: note = Description of Artificial Sequence: Synthetic oligonucleotide
  source 1..84: mol_type = other DNA
atctacatctgggcccctctggccggcacctgtggcgtgctgctgctgagcctggtcatcaccctgtactgcaaccaccggaat

SEQ ID NO: 93 - DNA, 81 bp, synthetic construct
  misc_feature 1..81: note = Description of Artificial Sequence: Synthetic oligonucleotide
  source 1..81: mol_type = other DNA
ttttgggtgctggtggtggttggtggagtcctggcttgctatagcttgctagtaacagtggcctttattattttctgggtg

SEQ ID NO: 94 - DNA, 126 bp, synthetic construct
  misc_feature 1..126: note = Description of Artificial Sequence: Synthetic polynucleotide
  source 1..126: mol_type = other DNA
aagagaggccggaagaaactgctgtacatcttcaagcagcccttcatgcggcccgtgcagaccacccaggaagaggacggctgcagctgccggttccccgaggaagaggaaggcggctgcgaactg

SEQ ID NO: 95 - DNA, 123 bp, synthetic construct
  misc_feature 1..123: note = Description of Artificial Sequence: Synthetic polynucleotide
  source 1..123: mol_type = other DNA
aggagcaagcggagcagaggcggccacagcgactacatgaacatgaccccccggaggcctggccccacccggaagcactaccagccctacgcccctcccagggacttcgccgcctaccggagc

SEQ ID NO: 96 - DNA, 72 bp, synthetic construct
  misc_feature 1..72: note = Description of Artificial Sequence: Synthetic oligonucleotide
  source 1..72: mol_type = other DNA
ctgtgcgcacgcccacgccgcagccccgcccaagaagatggcaaagtctacatcaacatgccaggcaggggc

SEQ ID NO: 97 - DNA, 156 bp, synthetic construct
  misc_feature 1..156: note = Description of Artificial Sequence: Synthetic polynucleotide
  source 1..156: mol_type = other DNA
tacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaa

SEQ ID NO: 98 - DNA, 336 bp, synthetic construct
  misc_feature 1..336: note = Description of Artificial Sequence: Synthetic polynucleotide
  source 1..336: mol_type = other DNA
cgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccaga

SEQ ID NO: 99 - DNA, 1005 bp, synthetic construct
  misc_feature 1..1005: note = Description of Artificial Sequence: Synthetic polynucleotide
  source 1..1005: mol_type = other DNA
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 100 - DNA, 732 bp, synthetic construct
  misc_feature 1..732: note = Description of Artificial Sequence: Synthetic polynucleotide
  source 1..732: mol_type = other DNA
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctctggtggcggtggctcgggcggtggtgggtcgggtggcggcggatctggtggcggtggctcgttttgggtgctggtggtggttggtggagtcctggcttgctatagcttgctagtaacagtggcctttattattttctgggtgaggagtaagaggagc

SEQ ID NO: 101 - DNA, 891 bp, synthetic construct
  misc_feature 1..891: note = Description of Artificial Sequence: Synthetic polynucleotide
  source 1..891: mol_type = other DNA
atgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctcct

SEQ ID NO: 102 - DNA, 789 bp, synthetic construct
  misc_feature 1..789: note = Description of Artificial Sequence: Synthetic polynucleotide
  source 1..789: mol_type = other DNA
atgaccacaccacggaactctgtgaatggcaccttcccagcagagccaatgaagggaccaatcgcaatgcagagcggacccaagcctctgtttcggagaatgagctccctggtgggcccaacccagtccttctttatgagagagtctaagacactgggcgccgtgcagatcatgaacggactgttccacatcgccctgggaggactgctgatgatcccagccggcatctacgcccctatctgcgtgaccgtgtggtaccctctgtggggcggcatcatgtatatcatctccggctctctgctggccgccacagagaagaacagcaggaagtgtctggtgaagggcaagatgatcatgaatagcctgtccctgtttgccgccatctctggcatgatcctgagcatcatggacatcctgaacatcaagatcagccacttcctgaagatggagagcctgaacttcatcagagcccacaccccttacatcaacatctataattgcgagcctgccaacccatccgagaagaattctccaagcacacagtactgttattccatccagtctctgttcctgggcatcctgtctgtgatgctgatctttgccttctttcaggagctggtcatcgccggcatcgtggagaacgagtggaagaggacctgcagccgccccaagtccaatatcgtgctgctgtccgccgaggagaagaaggagcagacaatcgagatcaaggaggaggtggtgggcctgaccgagacatctagccagcctaagaatgaggaggatatcgag

SEQ ID NO: 103 - protein, 395 aa, synthetic construct [sequence omitted]

SEQ ID NO: 104 - protein, 114 aa, synthetic construct
  REGION 1..114: note = Description of Artificial Sequence: Synthetic polypeptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 105 - protein, 237 aa, synthetic construct
  REGION 1..237: note = Description of Artificial Sequence: Synthetic polypeptide
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 106 - DNA, 1185 bp, synthetic construct
  misc_feature 1..1185: note = Description of Artificial Sequence: Synthetic polynucleotide
  source 1..1185: mol_type = other DNA
atggattggacctggattctgtttctggtggccgctgccacaagagtgcacagcaactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagctctggcggaggatctggaggaggcggatctggaggaggaggcagtggaggcggaggatctggcggaggatctctgcagattacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaggacctggagaattgttctcaccacctg

SEQ ID NO: 107 - DNA, 342 bp, synthetic construct
  misc_feature 1..342: note = Description of Artificial Sequence: Synthetic polynucleotide
  source 1..342: mol_type = other DNA
aactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagc

SEQ ID NO: 108 - DNA, 711 bp, synthetic construct
  misc_feature 1..711: note = Description of Artificial Sequence: Synthetic polynucleotide
  source 1..711: mol_type = other DNA
attacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaggacctggagaattgttctcaccacctg

SEQ ID NO: 109 - DNA, 339 bp, synthetic construct
  misc_feature 1..339: note = Description of Artificial Sequence: Synthetic polynucleotide
  source 1..339: mol_type = other DNA
caggtgcagctggtcgaaagcggaggaggagtggtccagccaggacgatccctgagactggattgtaaggcctctggaatcacattctctaacagtggaatgcactgggtgcgccaggcaccaggaaaaggactggagtgggtggccgtcatctggtacgacgggtcaaagcgatactatgcagatagcgtgaaaggaaggttcacaatttcacgcgacaacagcaagaatactctgtttctgcagatgaactctctgagagcagaggatactgccgtgtactattgtgctaccaatgacgattattgggggcagggaactctggtgaccgtcagttca

SEQ ID NO: 110 - DNA, 360 bp, synthetic construct
  misc_feature 1..360: note = Description of Artificial Sequence: Synthetic polynucleotide
  source 1..360: mol_type = other DNA
caggtgcagctggtccagagcggcgtggaagtcaagaaacccggggcctcagtgaaggtcagctgtaaagcttccggctacaccttcacaaactactatatgtattgggtgagacaggcaccaggacagggactggagtggatgggcgggattaaccctagtaatggaggcactaacttcaacgaaaagtttaaaaacagggtgaccctgaccacagattcaagcactaccacagcttacatggagctgaagtccctgcagtttgacgatacagccgtgtactattgtgctcggagagactacaggttcgatatgggctttgactattggggccaggggactaccgtgaccgtctcctct

SEQ ID NO: 111 - DNA, 321 bp, synthetic construct
  misc_feature 1..321: note = Description of Artificial Sequence: Synthetic polynucleotide
  source 1..321: mol_type = other DNA
gagatcgtcctgacacagagtccagcaactctgagcctgtcccccggcgaacgagctactctgtcctgccgggcatctcagagtgtgtctagttacctggcctggtatcagcagaagcccggccaggctcctaggctgctgatctacgacgccagcaacagagctaccgggattcctgccaggttctcaggcagcgggtccggaacagactttaccctgacaatctcaagcctggagcccgaagatttcgctgtgtactattgccagcagtcctctaattggcctcgcacctttggccaggggacaaaggtcgagatcaag

SEQ ID NO: 112 - DNA, 333 bp, synthetic construct
  misc_feature 1..333: note = Description of Artificial Sequence: Synthetic polynucleotide
  source 1..333: mol_type = other DNA
gagatcgtcctgactcagtccccagcaaccctgagtctgtcaccaggagaaagggcaaccctgagctgccgagcatccaagggggtgagcacatccggatactcttatctgcactggtaccagcagaaacccggacaggctcctcgactgctgatctacctggcatcttatctggagagtggcgtgcctgctcggttctctgggagtggatcaggcaccgattttacactgactatttctagtctggagccagaagatttcgcagtgtactattgccagcattctcgagacctgcccctgacatttggcgggggaactaaggtcgagatcaaa

SEQ ID NO: 113 - DNA, 1758 bp, synthetic construct
  misc_feature 1..1758: note = Description of Artificial Sequence: Synthetic polynucleotide
  source 1..1758: mol_type = other DNA
caggtgcagctggtcgaaagcggaggaggagtggtccagccaggacgatccctgagactggattgtaaggcctctggaatcacattctctaacagtggaatgcactgggtgcgccaggcaccaggaaaaggactggagtgggtggccgtcatctggtacgacgggtcaaagcgatactatgcagatagcgtgaaaggaaggttcacaatttcacgcgacaacagcaagaatactctgtttctgcagatgaactctctgagagcagaggatactgccgtgtactattgtgctaccaatgacgattattgggggcagggaactctggtgaccgtcagttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaaggtggaggtggttctggaggtggaggtagtatccctcctcacgtacagaagtccgtgaacaatgacatgattgtcactgacaataacggagccgtcaagtttcctcagctatgtaagttctgcgatgttcggttctccacatgcgataatcagaaaagctgtatgtctaattgcagtatcactagtatatgcgaaaaacctcaagaagtttgcgtcgccgtgtggcggaaaaatgatgaaaatatcacgcttgagactgtctgccatgatccaaagttaccctaccacgacttcatcttagaagacgccgcatcacccaagtgcattatgaaagagaaaaagaagccaggagaaacattctttatgtgctcatgctcctctgacgaatgcaacgacaacattatcttctctgaggagtataacacctcaaatccagac

SEQ ID NO: 114 - DNA, 1758 bp, synthetic construct
  misc_feature 1..1758: note = Description of Artificial Sequence: Synthetic polynucleotide
  source 1..1758: mol_type = other DNA
caggtgcagctggtcgaaagcggaggaggagtggtccagccaggacgatccctgagactggattgtaaggcctctggaatcacattctctaacagtggaatgcactgggtgcgccaggcaccaggaaaaggactggagtgggtggccgtcatctggtacgacgggtcaaagcgatactatgcagatagcgtgaaaggaaggttcacaatttcacgcgacaacagcaagaatactctgtttctgcagatgaactctctgagagcagaggatactgccgtgtactattgtgctaccaatgacgattattgggggcagggaactctggtgaccgtcagttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaaggtggaggtggttctggaggtggaggtagtatccctcctcacgtacagaagtccgtgaacaatgacatgattgtcactgacaataacggagccgtcaagtttcctcagctatgtaagttctgcgatgttcggttctccacatgcgataatcagaaaagctgtatgtctaattgcagtatcactagtatatgcgaaaaacctcaagaagtttgcgtcgccgtgtggcggaaaaatgatgaaaatatcacgcttgagactgtctgccatgatccaaagttaccctaccacgacttcatcttagaagacgccgcatcacccaagtgcattatgaaagagaaaaagaagccaggagaaacattctttatgtgctcatgctcctctgacgaatgcaacgacaacattatcttctctgaggagtataacacctcaaatccagac

SEQ ID NO: 115 - DNA, 1779 bp, synthetic construct
  misc_feature 1..1779: note = Description of Artificial Sequence: Synthetic polynucleotide
  source 1..1779: mol_type = other DNA
caggtgcagctggtccagagcggcgtggaagtcaagaaacccggggcctcagtgaaggtcagctgtaaagcttccggctacaccttcacaaactactatatgtattgggtgagacaggcaccaggacagggactggagtggatgggcgggattaaccctagtaatggaggcactaacttcaacgaaaagtttaaaaacagggtgaccctgaccacagattcaagcactaccacagcttacatggagctgaagtccctgcagtttgacgatacagccgtgtactattgtgctcggagagactacaggttcgatatgggctttgactattggggccaggggactaccgtgaccgtctcctctgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaaggtggaggtggttctggaggtggaggtagtatccctcctcacgtacagaagtccgtgaacaatgacatgattgtcactgacaataacggagccgtcaagtttcctcagctatgtaagttctgcgatgttcggttctccacatgcgataatcagaaaagctgtatgtctaattgcagtatcactagtatatgcgaaaaacctcaagaagtttgcgtcgccgtgtggcggaaaaatgatgaaaatatcacgcttgagactgtctgccatgatccaaagttaccctaccacgacttcatcttagaagacgccgcatcacccaagtgcattatgaaagagaaaaagaagccaggagaaacattctttatgtgctcatgctcctctgacgaatgcaacgacaacattatcttctctgaggagtataacacctcaaatccagac

SEQ ID NO: 116 - DNA, 1779 bp, synthetic construct
  misc_feature 1..1779: note = Description of Artificial Sequence: Synthetic polynucleotide
  source 1..1779: mol_type = other DNA
caggtgcagctggtccagagcggcgtggaagtcaagaaacccggggcctcagtgaaggtcagctgtaaagcttccggctacaccttcacaaactactatatgtattgggtgagacaggcaccaggacagggactggagtggatgggcgggattaaccctagtaatggaggcactaacttcaacgaaaagtttaaaaacagggtgaccctgaccacagattcaagcactaccacagcttacatggagctgaagtccctgcagtttgacgatacagccgtgtactattgtgctcggagagactacaggttcgatatgggctttgactattggggccaggggactaccgtgaccgtctcctctgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaaggtggaggtggttctggaggtggaggtagtatccctcctcacgtacagaagtccgtgaacaatgacatgattgtcactgacaataacggagccgtcaagtttcctcagctatgtaagttctgcgatgttcggttctccacatgcgataatcagaaaagctgtatgtctaattgcagtatcactagtatatgcgaaaaacctcaagaagtttgcgtcgccgtgtggcggaaaaatgatgaaaatatcacgcttgagactgtctgccatgatccaaagttaccctaccacgacttcatcttagaagacgccgcatcacccaagtgcattatgaaagagaaaaagaagccaggagaaacattctttatgtgctcatgctcctctgacgaatgcaacgacaacattatcttctctgaggagtataacacctcaaatccagac

SEQ ID NO: 117 - DNA, 981 bp, synthetic construct
  misc_feature 1..981: note = Description of Artificial Sequence: Synthetic polynucleotide
  source 1..981: mol_type = other DNA
gctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaa

SEQ ID NO: 118 - DNA, 321 bp, synthetic construct
  misc_feature 1..321: note = Description of Artificial Sequence: Synthetic polynucleotide
  source 1..321: mol_type = other DNA
gagatagttatgactcaaagccccgctacattatccctgtctccgggtgaacgggccaccctgtcatgccgggcttcacagtcagtgtcaagctatctggcatggtatcagcagaagcctggacaggccccaaggctactgatttatgacgccagcaaccgcgctacaggtattcctgctaggttctcagggtcaggctctggaaccgactttactctgactatctcctctcttgaacccgaggatttcgcggtgtactactgtcagcagtataataactggccacgcacattcggccagggcactaaagtcgaaattaag

SEQ ID NO: 119 - DNA, 321 bp, synthetic construct
  misc_feature 1..321: note = Description of Artificial Sequence: Synthetic polynucleotide
  source 1..321: mol_type = other DNA
gagatcgtactgactcagtctccagccacattgtccctgtccccaggggagcgcgccaccctgagctgtagagcttcacagtccgtcagttcttacctcgcgtggtatcagcaaaaacctggacaagctccgaggttgcttatctatgacgcctccaaccgcgccactggcataccagcaaggttcagcggatctgggtccggcacagattttaccctcactatttctagccttgagccggaagatttcgctgtttactactgccagcagcgatccaactggcccaagacattcggccagggaactaaagtggaaatcaaa

SEQ ID NO: 120 - DNA, 324 bp, synthetic construct
  misc_feature 1..324: note = Description of Artificial Sequence: Synthetic polynucleotide
  source 1..324: mol_type = other DNA
gaaattgtgttgacacagtctccagccaccctgtctttgtctccaggggaaagagccaccctctcctgcagggccagtcagagtgttagtagttacttagcctggtaccaacagaaacctggccaggctcccaggctcctcatctatgatgcatccaacagggccaacggcatcccagccaggttcagtggcagtgggtctgggacagacttcactctcaccatcagcagcctagagcctgaagatttcgcagtttattactgtcagcagagtagcaactggcctcggacgttcggccaagggaccaaggtggaaatcaaaaga

SEQ ID NO: 121 - DNA, 342 bp, synthetic construct
  misc_feature 1..342: note = Description of Artificial Sequence: Synthetic polynucleotide
  source 1..342: mol_type = other DNA
cggaacgtgctgacccagtccccccttagcctccccgtcacgcccggagagcccgcaagtatcagctgccgcagttcacaaagtctgagttcttctggatacacctatttggactggtatttgcagaagccagggcaatccccacagctcctgatatacctcgcaagctggagagatagcggagtacctgatcgcttttctggtagcggatctggtacggatttcactctgaagatttctagggtggaggcggaggacgtgggagtgtactactgtatgcaagccgagcagactcccggcccaggtaacacgttcggacaggggaccaaactggagattaag

SEQ ID NO: 122 - DNA, 336 bp, synthetic construct
  misc_feature 1..336: note = Description of Artificial Sequence: Synthetic polynucleotide
  source 1..336: mol_type = other DNA
gatgtggtaatgacccagtcacctctttcactgcctgtcactcccggagagccagcttcaatctcctgccgtagctctcaatcattgttgcacaccaatggatacaactacctccactggtatctccagaagcccggacaaagcccgcagctgctgatctacctgggcagctggcaggactccggggtgcccgaccgatttagcggcagtgggagcggcacggactttacactgaagatcagccgagtagaggcggaggacgtgggcgtttactactgtatgcaggcagagcagacccccagaaccttcggccagggcacccggctggaggtgaaa

SEQ ID NO: 123 - DNA, 339 bp, synthetic construct
  misc_feature 1..339: note = Description of Artificial Sequence: Synthetic polynucleotide
  source 1..339: mol_type = other DNA
caggtgcagttggttgaaagcggaggaggcgtggttcaacccggtagaagcctacggctgtcatgtgcggcctccggcttcacatttcgatcttacggaatgcactgggtcaggcaggcacccggcaagggtctggagtgggtcgccataattttctatgacggcagcaacaagtattacgccgacagtgttaaggggcggtttaccatcagcagagacaactctaaaaacactctttatctgcaaatgaactctctgcgggcagaggataccgctgtttactattgcgccagagatgacgactactgggggcagggtgccttggtgactgtgagcagc

SEQ ID NO: 124 - DNA, 342 bp, synthetic construct
  misc_feature 1..342: note = Description of Artificial Sequence: Synthetic polynucleotide
  source 1..342: mol_type = other DNA
gaggtccagttagtccaaagcggcggaggcgtagtgcaacctggcagaagcctgcggttatcgtgcgccgcaagcggcttcacctttagctcttatggtatgcactgggtcagacaggcccctgggaagggcctggagtgggtggccgtgatctggtatgacgggagcaacaagtattacgcggattccgtcaagggacggttcaccatatcccgcgataacagcaagaatactctttacttacagatgaacagcctgagggccgaggacaccgcagtatattattgcgctggcgaaggctttgactattggggtcagggcactctggtgactgtgagcagc

SEQ ID NO: 125 - DNA, 339 bp, synthetic construct
  misc_feature 1..339: note = Description of Artificial Sequence: Synthetic polynucleotide
  source 1..339: mol_type = other DNA
caggtgcagctggtggagtctgggggaggcgtggtccagcctgggaggtccctgagactcgactgtaaagcgtctggaatcaccttcagtaactctggcatgcactgggtccgccaggctccaggcaaggggctggagtgggtggcagttatttggtatgatggaagtaaaagatactatgcagactccgtgaagggccgattcaccatctccagagacaattccaagaacacgctgtttctgcaaatgaacagcctgagagccgaggacacggctgtgtattactgtgcgacaaacaacgactactggggccagggaaccctggtcaccgtctcctca

SEQ ID NO: 126 - DNA, 339 bp, synthetic construct
  misc_feature 1..339: note = Description of Artificial Sequence: Synthetic polynucleotide
  source 1..339: mol_type = other DNA
caggtgcagctggtggagtctgggggaggcgtggtccagcctgggaggtccctgagactcgactgtaaagcgtctggaatcaccttcagtaactctggcatgcactgggtccgccaggctccaggcaaggggctggagtgggtggcagttatttggtatgatggaagtaaaagatactatgcagactccgtgaagggccgattcaccatctccagagacaattccaagaacacgctgtttctgcaaatgaacagcctgagagccgaggacacggctgtgtattactgtgcgagaaacgacgactactggggccagggaaccctggtcaccgtctcctca

SEQ ID NO: 127 - DNA, 360 bp, synthetic construct
  misc_feature 1..360: note = Description of Artificial Sequence: Synthetic polynucleotide
  source 1..360: mol_type = other DNA
caggtacagctggtgcagagcggcgcagaggtgaagaagccaggcgcttctgtaaaggtatcctgcaaggcatccgggtatactttcaccggctattacatgcactgggttcgtcaggcacccggccagggactagaatggatgggggccatcaaccctaatagtggcggtactaactacgcacaaaagtttcaggggcgagtgaccatgactcgggatacctccatctccacggcatacatggagctgagtcgcttgcggtcagatgacactgcggtgtactactgcgctcgcaggcccgaccgagctaattggcactttgactactggggacagggtacactggtgaccgtgtcatca

SEQ ID NO: 128 - DNA, 360 bp, synthetic construct
  misc_feature 1..360: note = Description of Artificial Sequence: Synthetic polynucleotide
  source 1..360: mol_type = other DNA
caggtgcagctggtccagagcggcgcggaagtgaaaaagcccggcgcttccgtgaaggtttcttgcaaagcctctggatacacattcactggctattatatgcactgggtcagacaggcccccggccagggattggagtggatgggtgcaatcaaccccaattctggtgggaccaattacgcacagaaactccagggccgagtgacaatgaccaccgacacttctaccagcactgcctacatggagctgcggtctctgcgatcagacgacaccgctgtgtactattgtgcaagacacgggctgaagggcgacggctattttgactactggggacagggcacgctggttaccgtgagttcc

SEQ ID NO: 129 - DNA, 360 bp, synthetic construct
  misc_feature 1..360: note = Description of Artificial Sequence: Synthetic polynucleotide
  source 1..360: mol_type = other DNA
caggtccagctcgtgcaaagcggagtggaagtgaaaaagcctggcgcttccgtcaaggtcagctgtaaggccagcggatacacattcacaaactattacatgtactgggtgaggcaggctcccggacagggactggaatggatgggcggaatcaatccctccaacggaggcacaaactttaacgaaaagtttaagaatagagtcaccctcaccacagactccagcacaaccacagcctatatggaactgaaaagcctccagtttgacgataccgctgtgtattactgtgccaggagagattacaggttctacatgggattcgattactggggccaaggcacaaccgtcaccgtcagctcc

SEQ ID NO: 130 - DNA, 360 bp, synthetic construct
  misc_feature 1..360: note = Description of Artificial Sequence: Synthetic polynucleotide
  source 1..360: mol_type = other DNA
caggtccagctcgtgcaaagcggagtggaagtgaaaaagcctggcgcttccgtcaaggtcagctgtaaggccagcggatacacattcacaaactattacatgtactgggtgaggcaggctcccggacagggactggaatggatgggcggaatcaatccctccaacggaggcacaaactttaacgaaaagtttaagaatagagtcaccctcaccacagactccagcacaaccacagcctatatggaactgaaaagcctccagtttgacgataccgctgtgtattactgtgccaggagagattacaggttcaacatgggattcgattactggggccaaggcacaaccgtcaccgtcagctcc

SEQ ID NO: 131 - DNA, 360 bp, synthetic construct
  misc_feature 1..360: note = Description of Artificial Sequence: Synthetic polynucleotide
  source 1..360: mol_type = other DNA
caggtccagctcgtgcaaagcggagtggaagtgaaaaagcctggcgcttccgtcaaggtcagctgtaaggccagcggatacacattcacaaactattacatgtactgggtgaggcaggctcccggacagggactggaatggatgggcggaatccagccctccaacggaggcacaaactttaacgaaaagtttaagaatagagtcaccctcaccacagactccagcacaaccacagcctatatggaactgaaaagcctccagtttgacgataccgctgtgtattactgtgccaggagagattacaggttctacatgggattcgattactggggccaaggcacaaccgtcaccgtcagctcc

SEQ ID NO: 132 - DNA, 360 bp, synthetic construct
  misc_feature 1..360: note = Description of Artificial Sequence: Synthetic polynucleotide
  source 1..360: mol_type = other DNA
caggtccagctcgtgcaaagcggagtggaagtgaaaaagcctggcgcttccgtcaaggtcagctgtaaggccagcggatacacattcacaaactattacatgtactgggtgaggcaggctcccggacagggactggaatggatgggcggaatcgacccctccaacggaggcacaaactttaacgaaaagtttaagaatagagtcaccctcaccacagactccagcacaaccacagcctatatggaactgaaaagcctccagtttgacgataccgctgtgtattactgtgccaggtacgattacaggttcgatatgggattcgattactggggccaaggcacaaccgtcaccgtcagctcc

SEQ ID NO: 133 - DNA, 339 bp, synthetic construct
  misc_feature 1..339: note = Description of Artificial Sequence: Synthetic polynucleotide
  source 1..339: mol_type = other DNA
caggtgcagctggtggagtctgggggaggcgtggtccagcctgggaggtccctgagactcgactgtaaagcgtctggattcaccttcagtaactctggcatgcactgggtccgccaggctccaggcaaggggctggagtgggtggcagttatttggtatgatggaagtaaaagatactatgcagactccgtgaagggccgattcaccatctccagagacaattccaagaacacgctgtttctgcaaatgaacagcctgagagccgaggacacggctgtgtattactgtgcgacaaacaacgactactggggccagggaaccctggtcaccgtctcctca

SEQ ID NO: 134 - DNA, 339 bp, synthetic construct
  misc_feature 1..339: note = Description of Artificial Sequence: Synthetic polynucleotide
  source 1..339: mol_type = other DNA
gaggtccagttagtccaaagcggcggaggcgtagtgcaacctggcagaagcctgcggttatcgtgcgccgcaagcggcttcacctttagctcttatggtatgcactgggtcagacaggcccctgggaagggcctggagtgggtggccgtgatctggtatgacgggagcaacaagtattacgcggattccgtcaagggacggttcaccatatcccgcgataacagcaagaatactctttacttacagatgaacagcctgagggccgaggacaccgcagtatattattgcgctaccaataatgactattggggtcagggcactctggtgactgtgagcagc

SEQ ID NO: 135 - DNA, 336 bp, synthetic construct
  misc_feature 1..336: note = Description of Artificial Sequence: Synthetic polynucleotide
  source 1..336: mol_type = other DNA
gaggtgcaactggttcagtccggcgggggcgtcgtccagccggggcgcagtctgcgcttgagctgtgctgcctctgggattacctttagcaactccatgcattgggtgcggcaggcacccgggaagggactggaatgggtcgcagtgatctggtacgatggatcaaagcggtattacgccgactccgtcaaaggccggttcacaatcagccgcgacaacagcaaaaatactttatatcttcagatgaattcccttagggcagaggatactgctgtgtattactgcgctactaacaacgattattgggggcaggggacactagtcactgtttctagt

SEQ ID NO: 136 - DNA, 339 bp, synthetic construct
  misc_feature 1..339: note = Description of Artificial Sequence: Synthetic polynucleotide
  source 1..339: mol_type = other DNA
gaggtccagttagtccaaagcggcggaggcgtagtgcaacctggcagaagcctgcggttatcgtgcgccgcaagcggcttcacctttagcaattctggtatgcactgggtcagacaggcccctgggaagggcctggagtgggtggccgtgatctggtatgacgggagcaacaagtattacgcggattccgtcaagggacggttcaccatatcccgcgataacagcaagaatactctttacttacagatgaacagcctgagggccgaggacaccgcagtatattattgcgctaccaataatgactattggggtcagggcactctggtgactgtgagcagc

SEQ ID NO: 137 - DNA, 336 bp, synthetic construct
  misc_feature 1..336: note = Description of Artificial Sequence: Synthetic polynucleotide
  source 1..336: mol_type = other DNA
gaggtccagttagtccaaagcggcggaggcgtagtgcaacctggcagaagcctgcggttatcgtgcgccgcaagcggcttcacctttagctcttatggtatgcactgggtcagacaggcccctgggaagggcctggagtgggtggccgtgatctggtatgacgggagcaagtattacgcggattccgtcaagggacggttcaccatatcccgcgataacagcaagaatactctttacttacagatgaacagcctgagggccgaggacaccgcagtatattattgcgctaccaataatgactattggggtcagggcactctggtgactgtgagcagc

SEQ ID NO: 138 - DNA, 342 bp, synthetic construct
  misc_feature 1..342: note = Description of Artificial Sequence: Synthetic polynucleotide
  source 1..342: mol_type = other DNA
caggtgcagctggtggagtctgggggaggcgtggtccagcctgggaggtccctgagactcgactgtaaagcgtctggaatcaccttcagtaactctggcatgcactgggtccgccaggctccaggcaaggggctggagtgggtggcagttatttggtatgatggaagtaacaaaagatactatgcagactccgtgaagggccgattcaccatctccagagacaattccaagaacacgctgtttctgcaaatgaacagcctgagagccgaggacacggctgtgtattactgtgcgacaaacaacgactactggggccagggaaccctggtcaccgtctcctca

SEQ ID NO: 139 - DNA, 342 bp, synthetic construct
  misc_feature 1..342: note = Description of Artificial Sequence: Synthetic polynucleotide
  source 1..342: mol_type = other DNA
caggtgcagctggtggagtctgggggaggcgtggtccagcctgggaggtccctgagactcgactgtaaagcgtctggattcaccttcagtaactctggcatgcactgggtccgccaggctccaggcaaggggctggagtgggtggcagttatttggtatgatggaagtaacaaaagatactatgcagactccgtgaagggccgattcaccatctccagagacaattccaagaacacgctgtttctgcaaatgaacagcctgagagccgaggacacggctgtgtattactgtgcgacaaacaacgactactggggccagggaaccctggtcaccgtctcctca

SEQ ID NO: 140 - DNA, 336 bp, synthetic construct
  misc_feature 1..336: note = Description of Artificial Sequence: Synthetic polynucleotide
  source 1..336: mol_type = other DNA
gaggtgcaacttgtgcaaagcggcggcggagtcgtgcagcccggtcgatctcttcgcctgagttgtgctgccagcggcattacctttagcaattctggtatgcactgggtacgtcaggcccccggtaaggggctagaatgggtggctgtgatttggtacgatggttctaagtactacgccgacagcgttaaaggccgattcaccatcagtagagacaacagtaagaacaccctctacctccagatgaacagtctgcgagctgaagacactgctgtgtactactgtgccaccaacaacgactactggggacagggaaccctggtcaccgtgagtagt

SEQ ID NO: 141 - protein, 161 aa, synthetic construct
  REGION 1..161: note = Description of Artificial Sequence: Synthetic polypeptide
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 142 - protein, 112 aa, synthetic construct
  REGION 1..112: note = Description of Artificial Sequence: Synthetic polypeptide
PQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 143 - protein, 586 aa, synthetic construct
  REGION 1..586: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 144 - protein, 593 aa, synthetic construct
  REGION 1..593: note = Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 145 - protein, 593 aa, synthetic construct
  REGION 1..593: note = Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 146 - protein, 327 aa, synthetic construct
  REGION 1..327: note = Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 327 aa, synthetic construct
  REGION 1..327: note = Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 148 - protein, 108 aa, synthetic construct
  REGION 1..108: note = Description of Artificial Sequence: Synthetic polypeptide
EIVLTQSPATLSLSPGERATLSCRASQSVSSYLAWYQQKPGQAPRLLIYDASNRANGIPARFSGSGSGTDFTLTISSLEPEDFAVYYCQQSSNWPRTFGQGTKVEIKR

SEQ ID NO: 149 - protein, 113 aa, synthetic construct; identical to SEQ ID NO: 5
  REGION 1..113: note = Description of Artificial Sequence: Synthetic polypeptide

SEQ ID NO: 150 - protein, 113 aa, synthetic construct
  REGION 1..113: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCARNDDYWGQGTLVTVSS

SEQ ID NO: 151 - protein, 120 aa, synthetic construct; identical to SEQ ID NO: 3
  REGION 1..120: note = Description of Artificial Sequence: Synthetic polypeptide

SEQ ID NO: 152 - protein, 120 aa, synthetic construct; identical to SEQ ID NO: 4
  REGION 1..120: note = Description of Artificial Sequence: Synthetic polypeptide

SEQ ID NO: 153 - protein, 120 aa, synthetic construct
  REGION 1..120: note = Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFYMGFDYWGQGTTVTVSS

SEQ ID NO: 154 - protein, 120 aa, synthetic construct
  REGION 1..120: note = Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFNMGFDYWGQGTTVTVSS

SEQ ID NO: 155 - protein, 120 aa, synthetic construct
  REGION 1..120: note = Description of Artificial Sequence: Synthetic polypeptide
QVQLVQSGVEVKKPGASVKVSCKASGYTFTNYYMYWVRQAPGQGLEWMGGIQPSNGGTNFNEKFKNRVTLTTDSSTTTAYMELKSLQFDDTAVYYCARRDYRFYMGFDYWGQGTTVTVSS

SEQ ID NO: 156 - protein, 120 aa, synthetic construct
  REGION 1..120: note = Description of Artificial Sequence: Synthetic polypeptide
QVQLVQSGVEVKKPGASVKVSCKASGYTFTNYYMYWVRQAPGQGLEWMGGIDPSNGGTNFNEKFKNRVTLTTDSSTTTAYMELKSLQFDDTAVYYCARYDYRFDMGFDYWGQGTTVTVSS

SEQ ID NO: 157 - protein, 113 aa, synthetic construct
  REGION 1..113: note = Description of Artificial Sequence: Synthetic polypeptide
QVQLVESGGGVVQPGRSLRLDCKASGFTFSNSGMHWVRQAPGKGLEWVAVIWYDGSKRYYADSVKGRFTISRDNSKNTLFLQMNSLRAEDTAVYYCATNNDYWGQGTLVTVSS

SEQ ID NO: 158 - protein, 113 aa, synthetic construct
  REGION 1..113: note = Description of Artificial Sequence: Synthetic polypeptide
EVQLVQSGGGVVQPGRSLRLSCAASGFTFSSYGMHWVRQAPGKGLEWVAVIWYDGSNKYYADSVKGRFTISRDNSKNTLYLQMNSLRAEDTAVYYCATNNDYWGQGTLVTVSS

SEQ ID NO: 159 - protein, 112 aa, synthetic construct
  REGION 1..112: note = Description of Artificial Sequence: Synthetic polypeptide
EVQLVQSGGGVVQPGRSLRLSCAASGITFSNSMHWVRQAPGKGLEWVAVIWYDGSKRYYADSVKGRFTISRDNSKNTLYLQMNSLRAEDTAVYYCATNNDYWGQGTLVTVSS

SEQ ID NO: 160 - protein, 113 aa, synthetic construct
  REGION 1..113: note = Description of Artificial Sequence: Synthetic polypeptide
EVQLVQSGGGVVQPGRSLRLSCAASGFTFSNSGMHWVRQAPGKGLEWVAVIWYDGSNKYYADSVKGRFTISRDNSKNTLYLQMNSLRAEDTAVYYCATNNDYWGQGTLVTVSS

SEQ ID NO: 161 - protein, 112 aa, synthetic construct
  REGION 1..112: note = Description of Artificial Sequence: Synthetic polypeptide
EVQLVQSGGGVVQPGRSLRLSCAASGFTFSSYGMHWVRQAPGKGLEWVAVIWYDGSKYYADSVKGRFTISRDNSKNTLYLQMNSLRAEDTAVYYCATNNDYWGQGTLVTVSS

SEQ ID NO: 162 - protein, 114 aa, synthetic construct
  REGION 1..114: note = Description of Artificial Sequence: Synthetic polypeptide
QVQLVESGGGVVQPGRSLRLDCKASGITFSNSGMHWVRQAPGKGLEWVAVIWYDGSNKRYYADSVKGRFTISRDNSKNTLFLQMNSLRAEDTAVYYCATNNDYWGQGTLVTVSS

SEQ ID NO: 163 - protein, 45 aa, synthetic construct
  REGION 1..45: note = Description of Artificial Sequence: Synthetic polypeptide
QVQLVESGGGVVQPGRSLRLDCKASGFTFSNSGMHWVRQAPGKGL

SEQ ID NO: 164 - protein, 112 aa, synthetic construct
  REGION 1..112: note = Description of Artificial Sequence: Synthetic polypeptide
EVQLVQSGGGVVQPGRSLRLSCAASGITFSNSGMHWVRQAPGKGLEWVAVIWYDGSKYYADSVKGRFTISRDNSKNTLYLQMNSLRAEDTAVYYCATNNDYWGQGTLVTVSS

SEQ ID NO: 165 - protein, 116 aa, synthetic construct
  REGION 1..116: note = Description of Artificial Sequence: Synthetic polypeptide
DIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSDTFGGGTKLEIKRTVA

SEQ ID NO: 166 - protein, 124 aa, synthetic construct
  REGION 1..124: note = Description of Artificial Sequence: Synthetic polypeptide
  VARIANT 3: note = Any amino acid
  VARIANT 5: note = Any amino acid
QVXLXQSGAELVRPGTSVKVSCKASGYAFTNYLIEWVKQRPGQGLEWIGVNNPGSGGSNYNEKFKGKATLTADKSSSTAYMQLSSLTSDDSAVYFCARSGGFYFDYWGQGTTQSPSPQPKRRAH

SEQ ID NO: 167 - protein, 116 aa, synthetic construct
  REGION 1..116: note = Description of Artificial Sequence: Synthetic polypeptide
DIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIKRTVA

SEQ ID NO: 168 - protein, 124 aa, synthetic construct
  REGION 1..124: note = Description of Artificial Sequence: Synthetic polypeptide
EVQLVESGGGLVQPGGSLRLSCAASGYAFTNYLIEWVRQAPGKGLEWVGVNNPGSGGSNYNEKFKGRATISADNSKNTLYLQMNSLRAEDTAVYYCARSGGFYFDYWGQGTLVTVSSASTKGPS

SEQ ID NO: 169 - protein, 115 aa, synthetic construct
  REGION 1..115: note = Description of Artificial Sequence: Synthetic polypeptide
LARPGASVKMSCKTSGYTFTNYWMHWVRQRPGQGLEWIGTIYPGNSDTNYNQKFKDKAKLTAVTSATTAYMELSSLTNEDSAVYFCTREDSRSLYYNGWDYFDYWGQGTTLTVSS

SEQ ID NO: 170 - protein, 108 aa, synthetic construct
  REGION 1..108: note = Description of Artificial Sequence: Synthetic polypeptide
LTQSPASLAVSLGQRATISCRASESVDNYGISFLNWFQQKPGQPPKLLIYAASNQGSGVPARFSGSGSGTDFSLNIHPMEEDDTGMYFCQQSKEVPRTFGGGTKLEII

SEQ ID NO: 171 - protein, 121 aa, synthetic construct
  REGION 1..121: note = Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLWEVRALPSVYWGQGTLVTVSS

SEQ ID NO: 172 - protein, 109 aa, synthetic construct
  REGION 1..109: note = Description of Artificial Sequence: Synthetic polypeptide
SYELTQPPSVSVAPGQTARITCGANDIGSKSVHWYQQKAGQAPVLVVSEDIIRPSGIPERISGSNSGNTATLTISRVEAGDEADYYCQVWDRDSDQYVFGTGTKVTVLG

SEQ ID NO: 173 - protein, 120 aa, synthetic construct
  REGION 1..120: note = Description of Artificial Sequence: Synthetic polypeptide
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 174 - protein, 108 aa, synthetic construct
  REGION 1..108: note = Description of Artificial Sequence: Synthetic polypeptide
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 175 - protein, 120 aa, synthetic construct
  REGION 1..120: note = Description of Artificial Sequence: Synthetic polypeptide
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSS

SEQ ID NO: 176 - protein, 108 aa, synthetic construct; identical to SEQ ID NO: 174
  REGION 1..108: note = Description of Artificial Sequence: Synthetic polypeptide

SEQ ID NO: 177 - protein, 121 aa, synthetic construct
  REGION 1..121: note = Description of Artificial Sequence: Synthetic polypeptide
QVQLVQSGAEVKKPGSSVKVSCKASGGTFSTSFINWVRQAPGQGLEWMGGIIPIFDITNYAQKFQSRVTITADKSTSTAYMELSSLRSEDTAVYYCARGNGNYALDAMDYWGQGTLVTVSS

SEQ ID NO: 178 - protein, 108 aa, synthetic construct
  REGION 1..108: note = Description of Artificial Sequence: Synthetic polypeptide
EIVLTQSPGTLSLSPGERATLSCRASQSVSSSYFAWYQQKPGQAPRLLIYGASSRATGIPDRFSGSGSGTDFTLTISRLEPEDFAVYYCQQYYDSPITFGQGTRLEIK

SEQ ID NO: 179 - DNA, 342 bp, synthetic construct
  misc_feature 1..342: note = Description of Artificial Sequence: Synthetic polynucleotide
  source 1..342: mol_type = other DNA
gatataatgacacagagccccagctctctagctgtgagtgctggcgagaaggtgaccatgagctgtaagagcagtcaaagcgtgctgtacagttccaatcagaaaaattacctcgcatggtatcagaagccaggtcaaagccctaagctccttatctactgggcctcaacccgtgaaagtggagtgcctgacagatttactggttcagggagcggcaccgatttcactctgactattagctctgtgcaggcagaagaccttgccgtgtattactgtcaccagtatctgtcttcagacacgtttggaggtgggaccaaactagaaatcaaacgtactgtcgca

SEQ ID NO: 180 - DNA, 372 bp, synthetic construct
  misc_feature 1..372: note = Description of Artificial Sequence: Synthetic polynucleotide
  misc_difference 7..9: note = a, c, t, g, unknown or other
  misc_difference 13..15: note = a, c, t, g, unknown or other
  source 1..372: mol_type = other DNA
caggtgnnnctgnnncagagcggcgccgagctggtgaggcccggcaccagcgtgaaggtgagctgcaaggccagcggctacgccttcaccaactacctgatcgagtgggtgaagcagaggcccggccagggcctggagtggatcggcgtgaacaaccccggcagcggcggcagcaactacaacgagaagttcaagggcaaggccaccctgaccgccgacaagagcagcagcaccgcctacatgcagctgagcagcctgaccagcgacgacagcgccgtgtacttctgcgccaggagcggcggcttctacttcgactactggggccagggcaccacccagagccccagcccccagcccaagaggagggcccac

SEQ ID NO: 181 - DNA, 348 bp, synthetic construct
  misc_feature 1..348: note = Description of Artificial Sequence: Synthetic polynucleotide
  source 1..348: mol_type = other DNA
gacatccagatgacccagagccccagcagcctgagcgccagcgtgggcgacagggtgaccatcacctgcagggccagccagagcgtgctgtacagcagcaaccagaagaactacctggcctggtaccagcagaagcccggcaaggcccccaagctgctgatctactgggccagcaccagggagagcggcgtgcccagcaggttcagcggcagcggcagcggcaccgacttcaccctgaccatcagcagcctgcagcccgaggacttcgccacctactactgccaccagtacctgagcagcgacaccttcggccagggcaccaaggtggagatcaagaggaccgtggcc

SEQ ID NO: 182 - DNA, 372 bp, synthetic construct
  misc_feature 1..372: note = Description of Artificial Sequence: Synthetic polynucleotide
  source 1..372: mol_type = other DNA
gaggtgcagctggtggagagcggcggcggcctggtgcagcccggcggcagcctgaggctgagctgcgccgccagcggctacgccttcaccaactacctgatcgagtgggtgaggcaggcccccggcaagggcctggagtgggtgggcgtgaacaaccccggcagcggcggcagcaactacaacgagaagttcaagggcagggccaccatcagcgccgacaacagcaagaacaccctgtacctgcagatgaacagcctgagggccgaggacaccgccgtgtactactgcgccaggagcggcggcttctacttcgactactggggccagggcaccctggtgaccgtgagcagcgccagcaccaagggccccagc

SEQ ID NO: 183 - DNA, 345 bp, synthetic construct
  misc_feature 1..345: note = Description of Artificial Sequence: Synthetic polynucleotide
  source 1..345: mol_type = other DNA
ctggccaggcccggcgccagcgtgaagatgagctgcaagaccagcggctacaccttcaccaactactggatgcactgggtgaggcagaggcccggccagggcctggagtggatcggcaccatctaccccggcaacagcgacaccaactacaaccagaagttcaaggacaaggccaagctgaccgccgtgaccagcgccaccaccgcctacatggagctgagcagcctgaccaacgaggacagcgccgtgtacttctgcaccagggaggacagcaggagcctgtactacaacggctgggactacttcgactactggggccagggcaccaccctgaccgtgagcagc

SEQ ID NO: 184 - DNA, 324 bp, synthetic construct
  misc_feature 1..324: note = Description of Artificial Sequence: Synthetic polynucleotide
  source 1..324: mol_type = other DNA
ctgacccagagccccgccagcctggccgtgagcctgggccagagggccaccatcagctgcagggccagcgagagcgtggacaactacggcatcagcttcctgaactggttccagcagaagcccggccagccccccaagctgctgatctacgccgccagcaaccagggcagcggcgtgcccgccaggttcagcggcagcggcagcggcaccgacttcagcctgaacatccaccccatggaggaggacgacaccggcatgtacttctgccagcagagcaaggaggtgcccaggaccttcggcggcggcaccaagctggagatcatc

SEQ ID NO: 185 - DNA, 363 bp, synthetic construct
  misc_feature 1..363: note = Description of Artificial Sequence: Synthetic polynucleotide
  source 1..363: mol_type = other DNA
caggtgcagctggtgcagagcggcgccgaggtgaagaagcccggcagcagcgtgaaggtgagctgcaaggccagcggcggcaccttcagcagctacgccatcagctgggtgaggcaggcccccggccagggcctggagtggatgggcggcatcatccccatcttcggcaccgccaactacgcccagaagttccagggcagggtgaccatcaccgccgacgagagcaccagcaccgcctacatggagctgagcagcctgaggagcgaggacaccgccgtgtactactgcgccaggggcctgtgggaggtgagggccctgcccagcgtgtactggggccagggcaccctggtgaccgtgagcagc

SEQ ID NO: 186 - DNA, 327 bp, synthetic construct
  misc_feature 1..327: note = Description of Artificial Sequence: Synthetic polynucleotide
  source 1..327: mol_type = other DNA
agctacgagctgacccagccccccagcgtgagcgtggcccccggccagaccgccaggatcacctgcggcgccaacgacatcggcagcaagagcgtgcactggtaccagcagaaggccggccaggcccccgtgctggtggtgagcgaggacatcatcaggcccagcggcatccccgagaggatcagcggcagcaacagcggcaacaccgccaccctgaccatcagcagggtggaggccggcgacgaggccgactactactgccaggtgtgggacagggacagcgaccagtacgtgttcggcaccggcaccaaggtgaccgtgctgggc

SEQ ID NO: 187 - DNA, 360 bp, synthetic construct
  misc_feature 1..360: note = Description of Artificial Sequence: Synthetic polynucleotide
  source 1..360: mol_type = other DNA
caggtgcagctggtgcagagcggcgccgaggtgaagaagcccggcagcagcgtgaaggtgagctgcaaggccagcggctacaccttcagcagcaacgtgatcagctgggtgaggcaggcccccggccagggcctggagtggatgggcggcgtgatccccatcgtggacatcgccaactacgcccagaggttcaagggcagggtgaccatcaccgccgacgagagcaccagcaccacctacatggagctgagcagcctgaggagcgaggacaccgccgtgtactactgcgccagcaccctgggcctggtgctggacgccatggactactggggccagggcaccctggtgaccgtgagcagc

SEQ ID NO: 188 - DNA, 324 bp, synthetic construct
  misc_feature 1..324: note = Description of Artificial Sequence: Synthetic polynucleotide
  source 1..324: mol_type = other DNA
gagaccgtgctgacccagagccccggcaccctgagcctgagccccggcgagagggccaccctgagctgcagggccagccagagcctgggcagcagctacctggcctggtaccagcagaagcccggccaggcccccaggctgctgatctacggcgccagcagcagggcccccggcatccccgacaggttcagcggcagcggcagcggcaccgacttcaccctgaccatcagcaggctggagcccgaggacttcgccgtgtactactgccagcagtacgccgacagccccatcaccttcggccagggcaccaggctggagatcaag

SEQ ID NO: 189 - DNA, 360 bp, synthetic construct
  misc_feature 1..360: note = Description of Artificial Sequence: Synthetic polynucleotide
  source 1..360: mol_type = other DNA
caggtgcagctggtgcagagcggcgccgaggtgaagaagcccggcagcagcgtgaaggtgagctgcaaggccagcggctacaccttcagcagcaacgtgatcagctgggtgaggcaggcccccggccagggcctggagtggatgggcggcgtgatccccatcgtggacatcgccaactacgcccagaggttcaagggcagggtgaccatcaccgccgacgagagcaccagcaccacctacatggagctgagcagcctgaggagcgaggacaccgccgtgtactactgcgccctgcccagggccttcgtgctggacgccatggactactggggccagggcaccctggtgaccgtgagcagc

SEQ ID NO: 190 - DNA, 324 bp, synthetic construct; identical to SEQ ID NO: 188
  misc_feature 1..324: note = Description of Artificial Sequence: Synthetic polynucleotide
  source 1..324: mol_type = other DNA

SEQ ID NO: 191 - DNA, 363 bp, synthetic construct
  misc_feature 1..363: note = Description of Artificial Sequence: Synthetic polynucleotide
  source 1..363: mol_type = other DNA
caggtgcagctggtgcagagcggcgccgaggtgaagaagcccggcagcagcgtgaaggtgagctgcaaggccagcggcggcaccttcagcaccagcttcatcaactgggtgaggcaggcccccggccagggcctggagtggatgggcggcatcatccccatcttcgacatcaccaactacgcccagaagttccagagcagggtgaccatcaccgccgacaagagcaccagcaccgcctacatggagctgagcagcctgaggagcgaggacaccgccgtgtactactgcgccaggggcaacggcaactacgccctggacgccatggactactggggccagggcaccctggtgaccgtgagcagc

SEQ ID NO: 192 - DNA, 324 bp, synthetic construct
  misc_feature 1..324: note = Description of Artificial Sequence: Synthetic polynucleotide
  source 1..324: mol_type = other DNA
gagatcgtgctgacccagagccccggcaccctgagcctgagccccggcgagagggccaccctgagctgcagggccagccagagcgtgagcagcagctacttcgcctggtaccagcagaagcccggccaggcccccaggctgctgatctacggcgccagcagcagggccaccggcatccccgacaggttcagcggcagcggcagcggcaccgacttcaccctgaccatcagcaggctggagcccgaggacttcgccgtgtactactgccagcagtactacgacagccccatcaccttcggccagggcaccaggctggagatcaag

SEQ ID NO: 193-262 - skipped (70 entries)

SEQ ID NO: 263 - protein, 16 aa, synthetic construct
  REGION 1..16: note = Description of Artificial Sequence: Synthetic peptide
HANFCLGPCPYIWSLA

SEQ ID NO: 264 - protein, 15 aa, synthetic construct
  REGION 1..15: note = Description of Artificial Sequence: Synthetic peptide
FCLGPCPYIWSLDTA

SEQ ID NO: 265 - protein, 16 aa, synthetic construct
  REGION 1..16: note = Description of Artificial Sequence: Synthetic peptide
SNPYSAFQVDIIVDIA

SEQ ID NO: 266 - protein, 13 aa, synthetic construct
  REGION 1..13: note = Description of Artificial Sequence: Synthetic peptide
TSLDATMIWTMMA

SEQ ID NO: 267 - protein, 14 aa, synthetic construct
  REGION 1..14: note = Description of Artificial Sequence: Synthetic peptide
TSLDASIWAMMQNA

SEQ ID NO: 268 - DNA, 48 bp, synthetic construct
  misc_feature 1..48: note = Description of Artificial Sequence: Synthetic oligonucleotide
  source 1..48: mol_type = other DNA
cacgccaacttctgcctgggcccctgcccctacatctggagcctggcc

SEQ ID NO: 269 - DNA, 45 bp, synthetic construct
  misc_feature 1..45: note = Description of Artificial Sequence: Synthetic oligonucleotide
  source 1..45: mol_type = other DNA
ttctgcctgggcccctgcccctacatctggagcctggacaccgcc

SEQ ID NO: 270 - DNA, 48 bp, synthetic construct
  misc_feature 1..48: note = Description of Artificial Sequence: Synthetic oligonucleotide
  source 1..48: mol_type = other DNA
agcaacccctacagcgccttccaggtggacatcatcgtggacatcgcc

SEQ ID NO: 271 - DNA, 39 bp, synthetic construct
  misc_feature 1..39: note = Description of Artificial Sequence: Synthetic oligonucleotide
  source 1..39: mol_type = other DNA
accagcctggacgccaccatgatctggaccatgatggcc

SEQ ID NO: 272 - DNA, 42 bp, synthetic construct
  misc_feature 1..42: note = Description of Artificial Sequence: Synthetic oligonucleotide
  source 1..42: mol_type = other DNA
accagcctggacgccagcatctgggccatgatgcagaacgcc

SEQ ID NO: 273 - protein, 442 aa, synthetic construct
  REGION 1..442: note = Description of Artificial Sequence: Synthetic polypeptide
IDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHGIALPGDSLLRNFTLVTQHPEVIYTNQNVVLSKFETIFFTISGLIHYAPVFRDYVFRSMQEFYEDNVLYMEIRASLLPVYELSGEHHDEEWSVKTYQEVAQDFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 274 - protein, 442 aa, synthetic construct
  REGION 1..442: note = Description of Artificial Sequence: Synthetic polypeptide
IDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHGIALPGDSLLRNFTLVTQHPEVIYTNQNVVWSKFETIVFTISGLIHYAPVFRDYVFRSMQEFYEDNVLYMEIRARLLPVYELSGEHHDEEWSVKTYQEVAQDFVETHPEFIGIKIIYSDHRSKDVAVIAESIRMAMGLRIKFPTVVAGFDLAGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 275 - protein, 402 aa, synthetic construct
  REGION 1..402: note = Description of Artificial Sequence: Synthetic polypeptide
EMKEAMRTLIFPPSMHFFQAKHLIERSQVFNILRMMPKGAALHLHDIGIVTMDWLVRNVTYRPHGIALPGDSLLRNFTLVTQHPEVIYTNQNVVLSKFETIVFTISGLIHYAPVFRDYVFRSMQEFYEDNVLYMEIRARLLPVYELSGEHHDEEWSVKTYQEVAQDFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 276 - protein, 442 aa, synthetic construct
  REGION 1..442: note = Description of Artificial Sequence: Synthetic polypeptide
IDETRAHLLLKEKMMRLGGRLVLNTKEELANERLMTLKIAEMKEAMRTLIFPPSMHFFQAKHLIERSQVFNILRMMPKGAALHLHDIGIVTMDWLGGGGSGGGGSVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQEFVETHPEFIGIKIIYSDHRSRDVAVIAESIRMAMGLRIKFPTVVAGFDLSGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 277 - protein, 442 aa, synthetic construct
  REGION 1..442: note = Description of Artificial Sequence: Synthetic polypeptide
IDETRAHLLLKEKMMRLGGRLVLNTKEELANERLMTLKIAEMKEAMRTLIFPPSMHFFQAKHLIERSQVFNILRMMPKGAALHLHNIGIVTMDWLGGGGSGGGGSVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQDFVETHPEFIGIKIIYSDHRSYDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 278 - protein, 482 aa, synthetic construct
  REGION 1..482: note = Description of Artificial Sequence: Synthetic polypeptide
IDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 279 - protein, 482 aa, synthetic construct
  REGION 1..482: note = Description of Artificial Sequence: Synthetic polypeptide
IDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRAQLLPVYELSGEHHDEEWSVKTYQEVAQKFVETHPEFIGIKIIYNDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWDKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 280 - protein, 932 aa, synthetic construct
  REGION 1..932: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGGSGGGGSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 281 - protein, 932 aa, synthetic construct
  REGION 1..932: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGGSGGGGSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRAQLLPVYELSGEHHDEEWSVKTYQEVAQKFVETHPEFIGIKIIYNDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWDKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 282 - protein, 939 aa, synthetic construct
  REGION 1..939: note = Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGGSGGGGSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 283 - protein, 939 aa, synthetic construct
  REGION 1..939: note = Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGKGGGGSGGGGSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRAQLLPVYELSGEHHDEEWSVKTYQEVAQKFVETHPEFIGIKIIYNDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWDKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 284 - protein, 483 aa, synthetic construct
  REGION 1..483: note = Description of Artificial Sequence: Synthetic polypeptide
SIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 285 - DNA, 2796 bp, synthetic construct
  misc_feature 1..2796: note = Description of Artificial Sequence: Synthetic polynucleotide
  source 1..2796: mol_type = other DNA
caggtgcagctggtcgaaagcggaggaggagtggtccagccaggacgatccctgagactggattgtaaggcctctggaatcacattctctaacagtggaatgcactgggtgcgccaggcaccaggaaaaggactggagtgggtggccgtcatctggtacgacgggtcaaagcgatactatgcagatagcgtgaaaggaaggttcacaatttcacgcgacaacagcaagaatactctgtttctgcagatgaactctctgagagcagaggatactgccgtgtactattgtgctaccaatgacgattattgggggcagggaactctggtgaccgtcagttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaaggtggaggtggttctggaggtggaggtagtatcgacgaaaccagagcacacttactgctgaaagagaaaatgatgcgcctgggcgggagattggtgttaaatactaaggaagagctggcaaatgaaagactcatgacactgaagattgctgaaatgaaggaggcgatgaggacgctgatctttccgccttccatgcacttcttccaagctaaacacctgatcgaaagatcccaagtgtttaacatcctgaggatgatgcctaagggggccgctctgcaccttcacgatattgggattgtaacaatggactggctggtaaggaacgtgacatacagacctcattgccatatttgttttactccccgaggaatcatgcaattcaggtttgcccacccaactcctcggccaagcgagaagtgtagtaagtggattttgctggaagattaccgtaagcgcgtgcagaatgtgacagagtttgatgactccctgctccgcaattttaccctggtgacccagcaccccgaagttatatacactaaccaaaatgtcgtgtggtccaagtttgagacgatcttcttcacgatttcaggcttgatccactacgccccggtctttcgggattatgtgtttaggagtatgcaggagttttatgaggataatgttctgtacatggagatccgagcccggctgcttccagtctacgaactatccggcgaacaccatgacgaggaatggagcgtcaagacctatcaagaggtggcccagaagttcgtagaaacgcatccagagttcatcggtattaagattatctactctgatcaccgctcaaaggatgtggctgtcatcgccgagtctatacggatggccatgggcctgcggattaagttccctaccgtcgtcgccggattcgacctcgttgggcatgaggatactggccatagtctccatgactataaagaagcccttatgatcccagcaaaggacggagtgaagctgccctacttcttccacgcaggggagaccgactggcagggaacgagcatcgaccggaacatacttgatgcactcatgcttaataccacacgaatcggccacggcttcgctctctccaagcacccagccgtgagaacctacagctggaagaaggatatccccatcgaggtttgtcccatcagcaatcaggtgctgaaattggtgagtgacctgagaaaccacccagtcgcaacattaatggccactggccaccctatggtgatttcaagcgatgatccagccatgttcggagcaaaaggactcagttacgacttctatgaggtattcatgggtattggtggtatgaaggcagacctgcggactcttaagcagttggcaatgaactcaattaagtactctaccttattggagtctgaaaagaacacatttatggagatctggaaaaagcgctgggacaaattcatcgcagatgttgccacaaaa

SEQ ID NO: 286 - DNA, 2796 bp, synthetic construct
  misc_feature 1..2796: note = Description of Artificial Sequence: Synthetic polynucleotide
  source 1..2796: mol_type = other DNA
caggtgcagctggtcgaaagcggaggaggagtggtccagccaggacgatccctgagactggattgtaaggcctctggaatcacattctctaacagtggaatgcactgggtgcgccaggcaccaggaaaaggactggagtgggtggccgtcatctggtacgacgggtcaaagcgatactatgcagatagcgtgaaaggaaggttcacaatttcacgcgacaacagcaagaatactctgtttctgcagatgaactctctgagagcagaggatactgccgtgtactattgtgctaccaatgacgattattgggggcagggaactctggtgaccgtcagttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaaggtggaggtggttctggaggtggaggtagtatcgacgaaaccagagcacacttactgctgaaagagaaaatgatgcgcctgggcgggagattggtgttaaatactaaggaagagctggcaaatgaaagactcatgacactgaagattgctgaaatgaaggaggcgatgaggacgctgatctttccgccttccatgcacttcttccaagctaaacacctgatcgaaagatcccaagtgtttaacatcctgaggatgatgcctaagggggccgctctgcaccttcacgatattgggattgtaacaatggactggctggtaaggaacgtgacatacagacctcattgccatatttgttttactccccgaggaatcatgcaattcaggtttgcccacccaactcctcggccaagcgagaagtgtagtaagtggattttgctggaagattaccgtaagcgcgtgcagaatgtgacagagtttgatgactccctgctccgcaattttaccctggtgacccagcaccccgaagttatatacactaaccaaaatgtcgtgtggtccaagtttgagacgatcttcttcacgatttcaggcttgatccactacgccccggtctttcgggattatgtgtttaggagtatgcaggagttttatgaggataatgttctgtacatggagatccgagcccagctgcttccagtctacgaactatccggcgaacaccatgacgaggaatggagcgtcaagacctatcaagaggtggcccagaagttcgtagaaacgcatccagagttcatcggtattaagattatctacaatgatcaccgctcaaaggatgtggctgtcatcgccgagtctatacggatggccatgggcctgcggattaagttccctaccgtcgtcgccggattcgacctcgttgggcatgaggatactggccatagtctccatgactataaagaagcccttatgatcccagcaaaggacggagtgaagctgccctacttcttccacgcaggggagaccgactggcagggaacgagcatcgaccggaacatacttgatgcactcatgcttaataccacacgaatcggccacggcttcgctctctccaagcacccagccgtgagaacctacagctgggataaggatatccccatcgaggtttgtcccatcagcaatcaggtgctgaaattggtgagtgacctgagaaaccacccagtcgcaacattaatggccactggccaccctatggtgatttcaagcgatgatccagccatgttcggagcaaaaggactcagttacgacttctatgaggtattcatgggtattggtggtatgaaggcagacctgcggactcttaagcagttggcaatgaactcaattaagtactctaccttattggagtctgaaaagaacacatttatggagatctggaaaaagcgctgggacaaattcatcgcagatgttgccacaaaa

SEQ ID NO: 287 - DNA, 2817 bp, synthetic construct
  misc_feature 1..2817: note = Description of Artificial Sequence: Synthetic polynucleotide
  source 1..2817: mol_type = other DNA
caggtgcagctggtccagagcggcgtggaagtcaagaaacccggggcctcagtgaaggtcagctgtaaagcttccggctacaccttcacaaactactatatgtattgggtgagacaggcaccaggacagggactggagtggatgggcgggattaaccctagtaatggaggcactaacttcaacgaaaagtttaaaaacagggtgaccctgaccacagattcaagcactaccacagcttacatggagctgaagtccctgcagtttgacgatacagccgtgtactattgtgctcggagagactacaggttcgatatgggctttgactattggggccaggggactaccgtgaccgtctcctctgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaaggtggaggtggttctggaggtggaggtagtatcgacgaaaccagagcacacttactgctgaaagagaaaatgatgcgcctgggcgggagattggtgttaaatactaaggaagagctggcaaatgaaagactcatgacactgaagattgctgaaatgaaggaggcgatgaggacgctgatctttccgccttccatgcacttcttccaagctaaacacctgatcgaaagatcccaagtgtttaacatcctgaggatgatgcctaagggggccgctctgcaccttcacgatattgggattgtaacaatggactggctggtaaggaacgtgacatacagacctcattgccatatttgttttactccccgaggaatcatgcaattcaggtttgcccacccaactcctcggccaagcgagaagtgtagtaagtggattttgctggaagattaccgtaagcgcgtgcagaatgtgacagagtttgatgactccctgctccgcaattttaccctggtgacccagcaccccgaagttatatacactaaccaaaatgtcgtgtggtccaagtttgagacgatcttcttcacgatttcaggcttgatccactacgccccggtctttcgggattatgtgtttaggagtatgcaggagttttatgaggataatgttctgtacatggagatccgagcccggctgcttccagtctacgaactatccggcgaacaccatgacgaggaatggagcgtcaagacctatcaagaggtggcccagaagttcgtagaaacgcatccagagttcatcggtattaagattatctactctgatcaccgctcaaaggatgtggctgtcatcgccgagtctatacggatggccatgggcctgcggattaagttccctaccgtcgtcgccggattcgacctcgttgggcatgaggatactggccatagtctccatgactataaagaagcccttatgatcccagcaaaggacggagtgaagctgccctacttcttccacgcaggggagaccgactggcagggaacgagcatcgaccggaacatacttgatgcactcatgcttaataccacacgaatcggccacggcttcgctctctccaagcacccagccgtgagaacctacagctggaagaaggatatccccatcgaggtttgtcccatcagcaatcaggtgctgaaattggtgagtgacctgagaaaccacccagtcgcaacattaatggccactggccaccctatggtgatttcaagcgatgatccagccatgttcggagcaaaaggactcagttacgacttctatgaggtattcatgggtattggtggtatgaaggcagacctgcggactcttaagcagttggcaatgaactcaattaagtactctaccttattggagtctgaaaagaacacatttatggagatctggaaaaagcgctgggacaaattcatcgcagatgttgccacaaaa

SEQ ID NO: 288 - DNA, 2817 bp, synthetic construct
  misc_feature 1..2817: note = Description of Artificial Sequence: Synthetic polynucleotide
  source 1..2817: mol_type = other DNA
caggtgcagctggtccagagcggcgtggaagtcaagaaacccggggcctcagtgaaggtcagctgtaaagcttccggctacaccttcacaaactactatatgtattgggtgagacaggcaccaggacagggactggagtggatgggcgggattaaccctagtaatggaggcactaacttcaacgaaaagtttaaaaacagggtgaccctgaccacagattcaagcactaccacagcttacatggagctgaagtccctgcagtttgacgatacagccgtgtactattgtgctcggagagactacaggttcgatatgggctttgactattggggccaggggactaccgtgaccgtctcctctgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaaggtggaggtggttctggaggtggaggtagtatcgacgaaaccagagcacacttactgctgaaagagaaaatgatgcgcctgggcgggagattggtgttaaatactaaggaagagctggcaaatgaaagactcatgacactgaagattgctgaaatgaaggaggcgatgaggacgctgatctttccgccttccatgcacttcttccaagctaaacacctgatcgaaagatcccaagtgtttaacatcctgaggatgatgcctaagggggccgctctgcaccttcacgatattgggattgtaacaatggactggctggtaaggaacgtgacatacagacctcattgccatatttgttttactccccgaggaatcatgcaattcaggtttgcccacccaactcctcggccaagcgagaagtgtagtaagtggattttgctggaagattaccgtaagcgcgtgcagaatgtgacagagtttgatgactccctgctccgcaattttaccctggtgacccagcaccccgaagttatatacactaaccaaaatgtcgtgtggtccaagtttgagacgatcttcttcacgatttcaggcttgatccactacgccccggtctttcgggattatgtgtttaggagtatgcaggagttttatgaggataatgttctgtacatggagatccgagcccagctgcttccagtctacgaactatccggcgaacaccatgacgaggaatggagcgtcaagacctatcaagaggtggcccagaagttcgtagaaacgcatccagagttcatcggtattaagattatctacaatgatcaccgctcaaaggatgtggctgtcatcgccgagtctatacggatggccatgggcctgcggattaagttccctaccgtcgtcgccggattcgacctcgttgggcatgaggatactggccatagtctccatgactataaagaagcccttatgatcccagcaaaggacggagtgaagctgccctacttcttccacgcaggggagaccgactggcagggaacgagcatcgaccggaacatacttgatgcactcatgcttaataccacacgaatcggccacggcttcgctctctccaagcacccagccgtgagaacctacagctgggataaggatatccccatcgaggtttgtcccatcagcaatcaggtgctgaaattggtgagtgacctgagaaaccacccagtcgcaacattaatggccactggccaccctatggtgatttcaagcgatgatccagccatgttcggagcaaaaggactcagttacgacttctatgaggtattcatgggtattggtggtatgaaggcagacctgcggactcttaagcagttggcaatgaactcaattaagtactctaccttattggagtctgaaaagaacacatttatggagatctggaaaaagcgctgggacaaattcatcgcagatgttgccacaaaa

SEQ ID NO: 289 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 290 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 291 - protein, 327 aa, synthetic construct
  REGION 1..327: note = Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 292 - protein, 327 aa, synthetic construct
  REGION 1..327: note = Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 293 - protein, 9 aa, synthetic construct
  REGION 1..9: note = Description of Artificial Sequence: Synthetic peptide
GGGGSGGGS

SEQ ID NO: 294 - protein, 586 aa, synthetic construct
  REGION 1..586: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 295 - protein, 593 aa, synthetic construct
  REGION 1..593: note = Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 296 - protein, 218 aa, synthetic construct
  REGION 1..218: note = Description of Artificial Sequence: Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 297 - DNA, 1758 bp, synthetic construct
  misc_feature 1..1758: note = Description of Artificial Sequence: Synthetic polynucleotide
  source 1..1758: mol_type = other DNA
caggtgcagctggtcgaaagcggaggaggagtggtccagccaggacgatccctgagactggattgtaaggcctctggaatcacattctctaacagtggaatgcactgggtgcgccaggcaccaggaaaaggactggagtgggtggccgtcatctggtacgacgggtcaaagcgatactatgcagatagcgtgaaaggaaggttcacaatttcacgcgacaacagcaagaatactctgtttctgcagatgaactctctgagagcagaggatactgccgtgtactattgtgctaccaatgacgattattgggggcagggaactctggtgaccgtcagttcagctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaaggtggaggtggttctggaggtggaggtagtatccctcctcacgtacagaagtccgtgaacaatgacatgattgtcactgacaataacggagccgtcaagtttcctcagctatgtaagttctgcgatgttcggttctccacatgcgataatcagaaaagctgtatgtctaattgcagtatcactagtatatgcgaaaaacctcaagaagtttgcgtcgccgtgtggcggaaaaatgatgaaaatatcacgcttgagactgtctgccatgatccaaagttaccctaccacgacttcatcttagaagacgccgcatcacccaagtgcattatgaaagagaaaaagaagccaggagaaacattctttatgtgctcatgctcctctgacgaatgcaacgacaacattatcttctctgaggagtataacacctcaaatccagac

SEQ ID NO: 298 - DNA, 1779 bp, synthetic construct
  misc_feature 1..1779: note = Description of Artificial Sequence: Synthetic polynucleotide
  source 1..1779: mol_type = other DNA
caggtgcagctggtccagagcggcgtggaagtcaagaaacccggggcctcagtgaaggtcagctgtaaagcttccggctacaccttcacaaactactatatgtattgggtgagacaggcaccaggacagggactggagtggatgggcgggattaaccctagtaatggaggcactaacttcaacgaaaagtttaaaaacagggtgaccctgaccacagattcaagcactaccacagcttacatggagctgaagtccctgcagtttgacgatacagccgtgtactattgtgctcggagagactacaggttcgatatgggctttgactattggggccaggggactaccgtgaccgtctcctctgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggtaaaggtggaggtggttctggaggtggaggtagtatccctcctcacgtacagaagtccgtgaacaatgacatgattgtcactgacaataacggagccgtcaagtttcctcagctatgtaagttctgcgatgttcggttctccacatgcgataatcagaaaagctgtatgtctaattgcagtatcactagtatatgcgaaaaacctcaagaagtttgcgtcgccgtgtggcggaaaaatgatgaaaatatcacgcttgagactgtctgccatgatccaaagttaccctaccacgacttcatcttagaagacgccgcatcacccaagtgcattatgaaagagaaaaagaagccaggagaaacattctttatgtgctcatgctcctctgacgaatgcaacgacaacattatcttctctgaggagtataacacctcaaatccagac

SEQ ID NO: 299 - DNA, 642 bp, synthetic construct
  misc_feature 1..642: note = Description of Artificial Sequence: Synthetic polynucleotide
  source 1..642: mol_type = other DNA
gagatcgtcctgacacagagtccagcaactctgagcctgtcccccggcgaacgagctactctgtcctgccgggcatctcagagtgtgtctagttacctggcctggtatcagcagaagcccggccaggctcctaggctgctgatctacgacgccagcaacagagctaccgggattcctgccaggttctcaggcagcgggtccggaacagactttaccctgacaatctcaagcctggagcccgaagatttcgctgtgtactattgccagcagtcctctaattggcctcgcacctttggccaggggacaaaggtcgagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 300 - DNA, 654 bp, synthetic construct
  misc_feature 1..654: note = Description of Artificial Sequence: Synthetic polynucleotide
  source 1..654: mol_type = other DNA
gagatcgtcctgactcagtccccagcaaccctgagtctgtcaccaggagaaagggcaaccctgagctgccgagcatccaagggggtgagcacatccggatactcttatctgcactggtaccagcagaaacccggacaggctcctcgactgctgatctacctggcatcttatctggagagtggcgtgcctgctcggttctctgggagtggatcaggcaccgattttacactgactatttctagtctggagccagaagatttcgcagtgtactattgccagcattctcgagacctgcccctgacatttggcgggggaactaaggtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 301-309 - skipped (9 entries)

SEQ ID NO: 310 - protein, 7 aa, synthetic construct
  REGION 1..7: note = Description of Artificial Sequence: Synthetic peptide
STTYYWV

SEQ ID NO: 311 - protein, 16 aa, synthetic construct
  REGION 1..16: note = Description of Artificial Sequence: Synthetic peptide
SISYSGNTYYNPSLKS

SEQ ID NO: 312 - protein, 13 aa, synthetic construct
  REGION 1..13: note = Description of Artificial Sequence: Synthetic peptide
HLGYNGRYLPFDY

SEQ ID NO: 313 - protein, 7 aa, synthetic construct
  REGION 1..7: note = Description of Artificial Sequence: Synthetic peptide
SSTYYWG

SEQ ID NO: 314 - protein, 16 aa, synthetic construct
  REGION 1..16: note = Description of Artificial Sequence: Synthetic peptide
SISYSGSTYYNPSLKS

SEQ ID NO: 315 - protein, 7 aa, synthetic construct
  REGION 1..7: note = Description of Artificial Sequence: Synthetic peptide
STTYYWG

SEQ ID NO: 316 - protein, 16 aa, synthetic construct
  REGION 1..16: note = Description of Artificial Sequence: Synthetic peptide
SISYSGTTYYNPSLKS

SEQ ID NO: 317 - protein, 13 aa, synthetic construct
  REGION 1..13: note = Description of Artificial Sequence: Synthetic peptide
HLGYNSNWYPFDY

SEQ ID NO: 318 - protein, 5 aa, synthetic construct
  REGION 1..5: note = Description of Artificial Sequence: Synthetic peptide
SHAMS

SEQ ID NO: 319 - protein, 16 aa, synthetic construct
  REGION 1..16: note = Description of Artificial Sequence: Synthetic peptide
TITGGGGSIYYADSVK

SEQ ID NO: 320 - protein, 10 aa, synthetic construct
  REGION 1..10: note = Description of Artificial Sequence: Synthetic peptide
NRAGEGYFDY

SEQ ID NO: 321 - protein, 14 aa, synthetic construct
  REGION 1..14: note = Description of Artificial Sequence: Synthetic peptide
TGTSSDVGFYNYVS

SEQ ID NO: 322 - protein, 7 aa, synthetic construct
  REGION 1..7: note = Description of Artificial Sequence: Synthetic peptide
DVTNRPS

SEQ ID NO: 323 - protein, 10 aa, synthetic construct
  REGION 1..10: note = Description of Artificial Sequence: Synthetic peptide
SSYTSISTWV

SEQ ID NO: 324 - protein, 7 aa, synthetic construct
  REGION 1..7: note = Description of Artificial Sequence: Synthetic peptide
DVSNRPS

SEQ ID NO: 325 - protein, 10 aa, synthetic construct
  REGION 1..10: note = Description of Artificial Sequence: Synthetic peptide
SSYTNISTWV

SEQ ID NO: 326 - protein, 14 aa, synthetic construct
  REGION 1..14: note = Description of Artificial Sequence: Synthetic peptide
TGTSSDVGSYNRVS

SEQ ID NO: 327 - protein, 7 aa, synthetic construct
  REGION 1..7: note = Description of Artificial Sequence: Synthetic peptide
EVSNRPS

SEQ ID NO: 328 - protein, 10 aa, synthetic construct
  REGION 1..10: note = Description of Artificial Sequence: Synthetic peptide
SSYTSSSTWV

SEQ ID NO: 329 - protein, 11 aa, synthetic construct
  REGION 1..11: note = Description of Artificial Sequence: Synthetic peptide
GGDNIGNKDVH

SEQ ID NO: 330 - protein, 7 aa, synthetic construct
  REGION 1..7: note = Description of Artificial Sequence: Synthetic peptide
RDSNRPS

SEQ ID NO: 331 - protein, 8 aa, synthetic construct
  REGION 1..8: note = Description of Artificial Sequence: Synthetic peptide
QVWDSIWV

SEQ ID NO: 332 - protein, 10 aa, synthetic construct
  REGION 1..10: note = Description of Artificial Sequence: Synthetic peptide
SSYTSISTWV

SEQ ID NO: 333 - protein, 123 aa, synthetic construct
  REGION 1..123: note = Description of Artificial Sequence: Synthetic polypeptide
QLQLQESGPGLVKPSETLTLTCTVSGDSISSTTYYWVWIRQPPGKGLEWIGSISYSGNTYYNPSLKSRVTISVDTSKNHFSLKLSSVAATDTALYYCARHLGYNGRYLPFDYWGQGTLVTVSS

SEQ ID NO: 334 - protein, 123 aa, synthetic construct
  REGION 1..123: note = Description of Artificial Sequence: Synthetic polypeptide
QLQLQESGPGLVKPSETLSLTCTVSGGSISSSTYYWGWIRQPPGKGLEWIGSISYSGSTYYNPSLKSRVTISVDTSKNQFSLKLSSVTDADTAVYYCARHLGYNGRYLPFDYWGQGTLVTVSS

SEQ ID NO: 335 - protein, 123 aa, synthetic construct
  REGION 1..123: note = Description of Artificial Sequence: Synthetic polypeptide
QLQLQESGPGLVKPSETLTLTCTVSADSISSTTYYWVWIRQPPGKGLEWIGSISYSGSTYYNPSLKSRVTVSVDTSKNQFSLKLNSVAATDTALYYCARHLGYNGRYLPFDYWGQGTLVTVSS

SEQ ID NO: 336 - protein, 123 aa, synthetic construct
  REGION 1..123: note = Description of Artificial Sequence: Synthetic polypeptide
QLQLQESGPGLVKPSETLSLTCTVSGGSISSTTYYWGWIRQPPGKGLEWIGSISYSGTTYYNPSLKSRVTIPVDTSKNQISLKLSSVTAADTSLYYCARHLGYNSNWYPFDYWGQGTLVTVSS

SEQ ID NO: 337 - protein, 119 aa, synthetic construct
  REGION 1..119: note = Description of Artificial Sequence: Synthetic polypeptide
EVQLLESGGGLVQPGGSLRLSCAASGFTFSSHAMSWVRQAPGKGLEWVSTITGGGGSIYYADSVKGRFTISRDNSKNTLYLQMNSLRAEDTAVYYCAKNRAGEGYFDYWGQGTLVTVSS

SEQ ID NO: 338 - protein, 110 aa, synthetic construct
  REGION 1..110: note = Description of Artificial Sequence: Synthetic polypeptide
QSALTQPASVSGSPGQSITISCTGTSSDVGFYNYVSWYQQHPGKAPELMIYDVTNRPSGVSDRFSGSKSGNTASLTISGLQAEDEADYYCSSYTSISTWVFGGGTKLTVL

SEQ ID NO: 339 - protein, 110 aa, synthetic construct
  REGION 1..110: note = Description of Artificial Sequence: Synthetic polypeptide
QSALTQPASVSGSPGQSITISCTGTSSDVGFYNYVSWYQQHPGKAPEVMIYDVSNRPSGVSDRFSGSKSGNTASLTISGLQAEDEADYYCSSYTSISTWVFGGGTKLTVL

SEQ ID NO: 340 - protein, 110 aa, synthetic construct
  REGION 1..110: note = Description of Artificial Sequence: Synthetic polypeptide
QSALTQPASVSGSPGQSITISCTGTSSDVGFYNYVSWYQQHPGKAPELMIYDVSNRPSGVSDRFSGSKSGNTASLTISGLQAEDEADYYCSSYTNISTWVFGGGTKLTVL

SEQ ID NO: 341 - protein, 110 aa, synthetic construct
  REGION 1..110: note = Description of Artificial Sequence: Synthetic polypeptide
QSALTQPPSVSGSPGQSVTISCTGTSSDVGSYNRVSWYQQPPGTAPEVIIYEVSNRPSGVPDRFSGSKSGNTASLTISGLQAEDEADYYCSSYTSSSTWVFGGGTKLTVL

SEQ ID NO: 342 - protein, 105 aa, synthetic construct
  REGION 1..105: note = Description of Artificial Sequence: Synthetic polypeptide
SYELTQPLSVSVALGQTARITCGGDNIGNKDVHWYQQKPGQAPVLVIYRDSNRPSGIPEGFSGSNSGNTATLTISRAQAGDEADYYCQVWDSIWVFGGGTKLTVL

SEQ ID NO: 343 - protein, 110 aa, synthetic construct
  REGION 1..110: note = Description of Artificial Sequence: Synthetic polypeptide
QSALTQPASVSGSPGQSITISCTGTSSDVGFYNYVSWYQQHPGKAPELMIYDVSNRPSGVSDRFSGSKSGNTASLTISGLQAEDEADYYCSSYTSISTWVFGGGTKLTVL

SEQ ID NO: 344 - DNA, 21 bp, synthetic construct
  misc_feature 1..21: note = Description of Artificial Sequence: Synthetic oligonucleotide
  source 1..21: mol_type = other DNA
agtactacttactactgggtc

SEQ ID NO: 345 - DNA, 48 bp, synthetic construct
  misc_feature 1..48: note = Description of Artificial Sequence: Synthetic oligonucleotide
  source 1..48: mol_type = other DNA
agtatctcttatagtgggaacacctactacaatccgtccctcaagagt

SEQ ID NO: 346 - DNA, 39 bp, synthetic construct
  misc_feature 1..39: note = Description of Artificial Sequence: Synthetic oligonucleotide
  source 1..39: mol_type = other DNA
catctagggtataatgggaggtacctcccctttgactac

SEQ ID NO: 347 - DNA, 21 bp, synthetic construct
  misc_feature 1..21: note = Description of Artificial Sequence: Synthetic oligonucleotide
  source 1..21: mol_type = other DNA
agtagtacttactactggggc

SEQ ID NO: 348 - DNA, 48 bp, synthetic construct
  misc_feature 1..48: note = Description of Artificial Sequence: Synthetic oligonucleotide
  source 1..48: mol_type = other DNA
agtatctcttatagtgggagcacctactacaatccgtccctcaagagt

SEQ ID NO: 349 - DNA, 21 bp, synthetic construct
  misc_feature 1..21: note = Description of Artificial Sequence: Synthetic oligonucleotide
  source 1..21: mol_type = other DNA
agtactacttactactggggc

SEQ ID NO: 350 - DNA, 48 bp, synthetic construct
  misc_feature 1..48: note = Description of Artificial Sequence: Synthetic oligonucleotide
  source 1..48: mol_type = other DNA
agtatctcttatagtgggaccacctactacaacccgtccctcaagagt

SEQ ID NO: 351 - DNA, 39 bp, synthetic construct
  misc_feature 1..39: note = Description of Artificial Sequence: Synthetic oligonucleotide
  source 1..39: mol_type = other DNA
catctcgggtataacagcaactggtacccttttgactac

SEQ ID NO: 352 - DNA, 15 bp, synthetic construct
  misc_feature 1..15: note = Description of Artificial Sequence: Synthetic oligonucleotide
  source 1..15: mol_type = other DNA
agccatgccatgagc

SEQ ID NO: 353 - DNA, 51 bp, synthetic construct
  misc_feature 1..51: note = Description of Artificial Sequence: Synthetic oligonucleotide
  source 1..51: mol_type = other DNA
actattactggtggtggtggtagcatatactacgcagactccgtgaagggc

SEQ ID NO: 354 - DNA, 30 bp, synthetic construct
  misc_feature 1..30: note = Description of Artificial Sequence: Synthetic oligonucleotide
  source 1..30: mol_type = other DNA
aaccgcgctggggagggttactttgactac

SEQ ID NO: 355 - DNA, 42 bp, synthetic construct
  misc_feature 1..42: note = Description of Artificial Sequence: Synthetic oligonucleotide
  source 1..42: mol_type = other DNA
actggaaccagcagtgacgttggtttttataactatgtctcc

SEQ ID NO: 356 - DNA, 21 bp, synthetic construct
  misc_feature 1..21: note = Description of Artificial Sequence: Synthetic oligonucleotide
  source 1..21: mol_type = other DNA
gatgtcactaatcggccctca

SEQ ID NO: 357 - DNA, 30 bp, synthetic construct
  misc_feature 1..30: note = Description of Artificial Sequence: Synthetic oligonucleotide
  source 1..30: mol_type = other DNA
agctcatatacaagcatcagcacttgggtg

SEQ ID NO: 358 - DNA, 21 bp, synthetic construct
  misc_feature 1..21: note = Description of Artificial Sequence: Synthetic oligonucleotide
  source 1..21: mol_type = other DNA
gatgtcagtaatcggccctca

SEQ ID NO: 359 - DNA, 30 bp, synthetic construct
  misc_feature 1..30: note = Description of Artificial Sequence: Synthetic oligonucleotide
  source 1..30: mol_type = other DNA
agctcatatacaaacatcagcacttgggtg

SEQ ID NO: 360 - DNA, 42 bp, synthetic construct
  misc_feature 1..42: note = Description of Artificial Sequence: Synthetic oligonucleotide
  source 1..42: mol_type = other DNA
actggaaccagcagtgacgttggtagttataaccgtgtctcc

SEQ ID NO: 361 - DNA, 21 bp, synthetic construct
  misc_feature 1..21: note = Description of Artificial Sequence: Synthetic oligonucleotide
  source 1..21: mol_type = other DNA
gaggtcagtaatcggccctca

SEQ ID NO: 362 - DNA, 30 bp, synthetic construct
  misc_feature 1..30: note = Description of Artificial Sequence: Synthetic oligonucleotide
  source 1..30: mol_type = other DNA
agctcatatacaagcagcagcacttgggtg

SEQ ID NO: 363 - DNA, 33 bp, synthetic construct
  misc_feature 1..33: note = Description of Artificial Sequence: Synthetic oligonucleotide
  source 1..33: mol_type = other DNA
gggggagacaacattggaaataaagatgtgcac

SEQ ID NO: 364 - DNA, 21 bp, synthetic construct
  misc_feature 1..21: note = Description of Artificial Sequence: Synthetic oligonucleotide
  source 1..21: mol_type = other DNA
agggatagcaaccggccctct

SEQ ID NO: 365 - DNA, 24 bp, synthetic construct
  misc_feature 1..24: note = Description of Artificial Sequence: Synthetic oligonucleotide
  source 1..24: mol_type = other DNA
caggtgtgggacagcatttgggtg

SEQ ID NO: 366 - DNA, 30 bp, synthetic construct; identical to SEQ ID NO: 357
  misc_feature 1..30: note = Description of Artificial Sequence: Synthetic oligonucleotide
  source 1..30: mol_type = other DNA

SEQ ID NO: 367 - DNA, 369 bp, synthetic construct
  misc_feature 1..369: note = Description of Artificial Sequence: Synthetic polynucleotide
  source 1..369: mol_type = other DNA
cagctgcagctgcaggagtcgggcccaggactggtgaagccttcggagaccctgaccctcacctgcactgtctctggtgactccatcagcagtactacttactactgggtctggatccgccagcccccagggaagggactggagtggattgggagtatctcttatagtgggaacacctactacaatccgtccctcaagagtcgagtcaccatatccgtagacacgtccaagaaccacttctccctgaagctgagttctgtggccgccacagacacggctctatattactgtgcgagacatctagggtataatgggaggtacctcccctttgactactggggccagggaaccctggtcaccgtctcctcc

SEQ ID NO: 368 - DNA, 369 bp, synthetic construct
  misc_feature 1..369: note = Description of Artificial Sequence: Synthetic polynucleotide
  source 1..369: mol_type = other DNA
cagctgcagctgcaggagtcgggcccaggactggtgaagccttcggagaccctgtccctcacctgcactgtctctggtggctccatcagcagtagtacttactactggggctggatccgccagcccccagggaagggactggagtggattgggagtatctcttatagtgggagcacctactacaatccgtccctcaagagtcgagtcaccatatccgtagacacgtccaagaaccagttctccctgaagctgagctctgtgaccgacgcagacacggctgtgtattactgtgcgagacatctagggtataatgggaggtacctcccctttgactactggggccagggaaccctggtcaccgtctcctcc

SEQ ID NO: 369 - DNA, 369 bp, synthetic construct
  misc_feature 1..369: note = Description of Artificial Sequence: Synthetic polynucleotide
  source 1..369: mol_type = other DNA
cagctgcagctgcaggagtcgggcccaggactggtgaagccttcggagaccctgaccctcacctgcactgtctctgctgactccatcagcagtactacttactactgggtctggatccgccagcccccagggaagggactggagtggattgggagtatctcttatagtgggagcacctactacaatccgtccctcaagagtcgagtcaccgtatccgtagacacgtccaagaaccagttctccctgaagctgaactctgtggccgccacagacacggctctatattactgtgcgagacatctagggtataatgggaggtacctcccctttgactactggggccagggaaccctggtcaccgtctcctcc

SEQ ID NO: 370 - DNA, 369 bp, synthetic construct
  misc_feature 1..369: note = Description of Artificial Sequence: Synthetic polynucleotide
  source 1..369: mol_type = other DNA
cagctgcagctgcaggagtcgggcccaggactggtgaagccctcggagaccctgtccctcacctgcactgtctctggtggctccatcagcagtactacttactactggggctggatccgccagcccccagggaaggggctggagtggattgggagtatctcttatagtgggaccacctactacaacccgtccctcaagagtcgagtcaccatccccgtagacacgtccaagaaccagatctccctgaaactgagctctgtgaccgccgcagacacgtctttgtattattgtgcgagacatctcgggtataacagcaactggtacccttttgactactggggccagggaaccctggtcaccgtctcctca

SEQ ID NO: 371 - DNA, 357 bp, synthetic construct
  misc_feature 1..357: note = Description of Artificial Sequence: Synthetic polynucleotide
  source 1..357: mol_type = other DNA
gaggtgcagctgttggagtctgggggaggcttggtacagcctggggggtccctgagactgtcctgcgcagcctctggattcacctttagcagccatgccatgagctgggtccgccaggctccagggaaggggctggagtgggtctcaactattactggtggtggtggtagcatatactacgcagactccgtgaagggccggttcaccatctccagagacaattccaagaacacgctgtatctgcaaatgaacagcctgagagccgaggacacggccgtatattattgtgcgaaaaaccgcgctggggagggttactttgactactggggccagggaaccctggtcaccgtctcctca

SEQ ID NO: 372 - DNA, 330 bp, synthetic construct
  misc_feature 1..330: note = Description of Artificial Sequence: Synthetic polynucleotide
  source 1..330: mol_type = other DNA
cagtctgccctgactcagcctgcctccgtgtctgggtctcctggacagtcgatcaccatctcctgcactggaaccagcagtgacgttggtttttataactatgtctcctggtaccaacagcacccaggcaaagcccccgaactcatgatttatgatgtcactaatcggccctcaggggtttctgatcgcttctctggctccaagtctggcaacacggcctccctgaccatctctgggctccaggctgaggacgaggctgattattactgcagctcatatacaagcatcagcacttgggtgttcggcggagggaccaagctgaccgtccta

SEQ ID NO: 373 - DNA, 330 bp, synthetic construct
  misc_feature 1..330: note = Description of Artificial Sequence: Synthetic polynucleotide
  source 1..330: mol_type = other DNA
cagtctgccctgactcagcctgcctccgtgtctgggtctcctggacagtcgatcaccatctcctgcactggaaccagcagtgacgttggtttttataactatgtctcctggtaccaacagcacccaggcaaagcccccgaagtcatgatttatgatgtcagtaatcggccctcaggggtttctgatcgcttctctggctccaagtctggcaacacggcctccctgactatctctgggctccaggctgaggacgaggctgattattactgcagctcatatacaagcatcagcacttgggtgttcggcggagggaccaagctgactgtccta

SEQ ID NO: 374 - DNA, 330 bp, synthetic construct
  misc_feature 1..330: note = Description of Artificial Sequence: Synthetic polynucleotide
  source 1..330: mol_type = other DNA
cagtctgccctgactcagcctgcctccgtgtctgggtctcctggacagtcgatcaccatctcctgcactggaaccagcagtgacgttggtttttataactatgtctcctggtaccaacagcacccaggcaaagcccccgaactcatgatttatgatgtcagtaatcggccctcaggggtttctgatcgcttctctggctccaagtctggcaacacggcctccctgaccatctctgggctccaggctgaggacgaggctgattattactgcagctcatatacaaacatcagcacttgggtgttcggcggagggaccaagctgaccgtccta

SEQ ID NO: 375 - DNA, 330 bp, synthetic construct
  misc_feature 1..330: note = Description of Artificial Sequence: Synthetic polynucleotide
  source 1..330: mol_type = other DNA
cagtcggccctgactcagcctccctccgtgtccgggtctcctggacagtcagtcaccatctcctgcactggaaccagcagtgacgttggtagttataaccgtgtctcctggtaccagcagcccccaggcacagcccccgaagtcattatttatgaggtcagtaatcggccctcaggggtccctgatcgcttctctgggtccaagtctggcaacacggcctccctgaccatctctgggctccaggctgaggacgaggctgattattactgcagctcatatacaagcagcagcacttgggtgttcggcggagggaccaagctgaccgtccta

SEQ ID NO: 376 - DNA, 315 bp, synthetic construct
  misc_feature 1..315: note = Description of Artificial Sequence: Synthetic polynucleotide
  source 1..315: mol_type = other DNA
tcctatgagctgactcagccactctcagtgtcagtggccctgggacagacggccaggattacctgtgggggagacaacattggaaataaagatgtgcactggtaccagcagaagccaggccaggcccctgtgctggtcatctatagggatagcaaccggccctctgggatccctgagggattctctggctccaactcggggaacacggccaccctgaccatcagcagagcccaagccggggatgaggctgactattactgtcaggtgtgggacagcatttgggtgttcggcggagggaccaagctgaccgtccta

SEQ ID NO: 377 - DNA, 330 bp, synthetic construct
  misc_feature 1..330: note = Description of Artificial Sequence: Synthetic polynucleotide
  source 1..330: mol_type = other DNA
cagtctgccctgactcagcctgcctccgtgtctgggtctcctggacagtcgatcaccatctcctgcactggaaccagcagtgacgttggtttttataactatgtctcctggtaccaacagcacccaggcaaagcccccgaactcatgatttatgatgtcagtaatcggccctcaggggtttctgatcgcttctctggctccaagtctggcaacacggcctccctgaccatctctgggctccaggctgaggacgaggctgattattactgcagctcatatacaagcatcagcacttgggtgttcggcggagggaccaagctgaccgtccta

SEQ ID NO: 378 - protein, 5 aa, synthetic construct
  REGION 1..5: note = Description of Artificial Sequence: Synthetic peptide
SYAIS

SEQ ID NO: 379 - protein, 16 aa, synthetic construct
  REGION 1..16: note = Description of Artificial Sequence: Synthetic peptide
GIIPIFDTANYAQKFQ

SEQ ID NO: 380 - protein, 14 aa, synthetic construct
  REGION 1..14: note = Description of Artificial Sequence: Synthetic peptide
PGLAAAYDTGSLDY

SEQ ID NO: 381 - protein, 11 aa, synthetic construct
  REGION 1..11: note = Description of Artificial Sequence: Synthetic peptide
RASQSVRSYLA

SEQ ID NO: 382 - protein, 7 aa, synthetic construct
  REGION 1..7: note = Description of Artificial Sequence: Synthetic peptide
DASNRAT

SEQ ID NO: 383 - protein, 9 aa, synthetic construct
  REGION 1..9: note = Description of Artificial Sequence: Synthetic peptide
QQRNYWPLT

SEQ ID NO: 384 - protein, 123 aa, synthetic construct
  REGION 1..123: note = Description of Artificial Sequence: Synthetic polypeptide
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSS

SEQ ID NO: 385 - protein, 107 aa, synthetic construct
  REGION 1..107: note = Description of Artificial Sequence: Synthetic polypeptide
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIK

SEQ ID NO: 386 - protein, 450 aa, synthetic construct
  REGION 1..450: note = Description of Artificial Sequence: Synthetic polypeptide
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 387 - protein, 214 aa, synthetic construct
  REGION 1..214: note = Description of Artificial Sequence: Synthetic polypeptide
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 388 - protein, 265 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 389 - skipped

SEQ ID NO: 390 - protein, 113 aa, synthetic construct
  REGION 1..113: note = Description of Artificial Sequence: Synthetic polypeptide
QVQLQESGPGLVKPSQTLSLTCTVSGYSIVSHYYWSWIRQHPGKGLEWIGYISSDGSNYYNPSLKSLVTISVDTSKNQFSLKLSSVTAADTAVYYCVRGVDYWGQGTMVTVSS

SEQ ID NO: 391 - protein, 113 aa, synthetic construct
  REGION 1..113: note = Description of Artificial Sequence: Synthetic polypeptide
QVQLQESGPGLVKPSDTLSLTCAVSGYSIVSHYYWGWIRQPPGKGLEWIGYISSDGSNYYNPSLKSRVTMSVDTSKNQFSLKLSSVTAVDTAVYYCVRGVDYWGQGTMVTVSS

SEQ ID NO: 392 - protein, 113 aa, synthetic construct
  REGION 1..113: note = Description of Artificial Sequence: Synthetic polypeptide
EVQLVESGGGLVQPGGSLRLSCAASGYSIVSHYYMSWVRQAPGKGLEWVSVISSDGSNYYADSVKGRFTISRDNSKNTLYLQMNSLRAEDTAVYYCVRGVDYWGQGTLVTVSS

SEQ ID NO: 393 - protein, 113 aa, synthetic construct
  REGION 1..113: note = Description of Artificial Sequence: Synthetic polypeptide
EVQLVESGGGLVQPGRSLRLSCAASGYSIVSHYYMHWVRQAPGKGLEWVSAISSDGSNEYADSVEGRFTISRDNAKNSLYLQMNSLRAEDTAVYYCVRGVDYWGQGTLVTVSS

SEQ ID NO: 394 - protein, 113 aa, synthetic construct
  REGION 1..113: note = Description of Artificial Sequence: Synthetic polypeptide
QVQLQESGPGLVKPSQTLSLTCTVSGYSIVSHYYWSWIRQHPGKGLEWIGYISSDGSNEYNPSLKSLVTISVDTSKNQFSLKLSSVTAADTAVYFCVRGVDYWGQGTMVTVSS

SEQ ID NO: 395 - protein, 113 aa, synthetic construct
  REGION 1..113: note = Description of Artificial Sequence: Synthetic polypeptide
QVQLVQSGAEVKKPGSSVKVSCKASGYSIVSHYYISWVRQAPGQGLEWMGGISSDGSNNYAQKFQGRVTITADESTSTAYMELSSLRSEDTAVYYCVRGVDYWGQGTLVTVSS

SEQ ID NO: 396 - protein, 113 aa, synthetic construct
  REGION 1..113: note = Description of Artificial Sequence: Synthetic polypeptide
QVQLQESGPGLVKPSETLSLTCTVSGYSIVSHYYWSWIRQPPGKGLEWIGYISSDGSNNYNPSLKSRVTISVDTSKNQFSLKLSSVTAADTAVYYCVRGVDYWGQGTTVTVSS

SEQ ID NO: 397 - protein, 113 aa, synthetic construct
  REGION 1..113: note = Description of Artificial Sequence: Synthetic polypeptide
QVQLQESGPGLVKPSDTLSLTCAVSGYSIVSHYYWHWIRQPPGKGLEWMGYISSDGSNDFNPSLKTRITISRDTSKNQFSLKLSSVTAVDTAVYYCVRGVDYWGQGTLVTVSS

SEQ ID NO: 398 - protein, 113 aa, synthetic construct
  REGION 1..113: note = Description of Artificial Sequence: Synthetic polypeptide
QVQLQESGPGLVKPSQTLSLTCAVYGYSIVSHYYWSWIRQPPGKGLEWIGEISSDGSNNYNPSLKSRVTISVDTSKNQFSLKLSSVTAADTAVYYCVRGVDYWGQGTLVTVSS

SEQ ID NO: 399 - protein, 113 aa, synthetic construct
  REGION 1..113: note = Description of Artificial Sequence: Synthetic polypeptide
QVQLQESGPGLVKPSETLSLTCAVSGYSIVSHYYWGWIRQPPGKGLEWIGSISSDGSNYYNPSLKSRVTISVDTSKNQFSLKLSSVTAADTAVYYCVRGVDYWGQGTLVTVSS

SEQ ID NO: 400 - protein, 113 aa, synthetic construct
  REGION 1..113: note = Description of Artificial Sequence: Synthetic polypeptide
QVQLVESGGGVVQPGRSLRLSCAASGYSIVSHYYWNWVRQAPGKGLEWVAYISSDGSNEYNPSLKNRFTISRDNSKNTLYLQMNSLRAEDTAVYYCVRGVDYWGQGTTVTVSS

SEQ ID NO: 401 - protein, 113 aa, synthetic construct
  REGION 1..113: note = Description of Artificial Sequence: Synthetic polypeptide
QVQLQESGPGLVKPSQTLSLTCTVSGYSIVSHYYWNWIRQHPGKGLEWIGYISSDGSNEYNPSLKNLVTISVDTSKNQFSLKLSSVTAADTAVYYCVRGVDYWGQGTMVTVSS

SEQ ID NO: 402 - protein, 113 aa, synthetic construct
  REGION 1..113: note = Description of Artificial Sequence: Synthetic polypeptide
QVQLQESGPGLVKPSDTLSLTCAVSGYSIVSHYYWNWIRQPPGKGLEWIGYISSDGSNEYNPSLKNRVTMSVDTSKNQFSLKLSSVTAVDTAVYYCVRGVDYWGQGTMVTVSS

SEQ ID NO: 403 - protein, 113 aa, synthetic construct
  REGION 1..113: note = Description of Artificial Sequence: Synthetic polypeptide
EVQLLESGGGLVQPGGSLRLSCAASGYSIVSHYYWNWVRQAPGKGLEWVSYISSDGSNEYNPSLKNRFTISRDNSKNTLYLQMNSLRAEDTAVYYCVRGVDYWGQGTLVTVSS

SEQ ID NO: 404 - DNA, 339 bp, synthetic construct
  misc_feature 1..339: note = Description of Artificial Sequence: Synthetic polynucleotide
  source 1..339: mol_type = other DNA
caggtgcaactgcaggaatcaggtccaggcttggtcaagccatcgcagactcttagtctgacatgcaccgtgagtggctatagcatcgtgtcgcactattattggtcttggatcaggcagcatccaggaaagggactggagtggatcgggtacattagcagcgatgggagcaactattacaacccatctctgaagtccctggtaactattagcgtggatacaagcaaaaatcagttttcattaaagctctcttcagtgaccgcagctgataccgccgtctattattgcgtgcggggggtggactactggggtcagggcaccatggttactgtgtcatca

SEQ ID NO: 405 - DNA, 339 bp, synthetic construct
  misc_feature 1..339: note = Description of Artificial Sequence: Synthetic polynucleotide
  source 1..339: mol_type = other DNA
caggtacagctgcaggagagtggccctggtttagtaaagccatcagatacactttcacttacctgcgccgtgtctggttattctatcgtgagccactattactggggatggatccgccagccccctggcaaaggtcttgagtggattggctatataagttcggatggcagtaactattacaatccttctctgaagagccgtgtcactatgagcgtggacactagcaaaaaccagttcagcctgaagctgtcctccgtcaccgccgtagacaccgctgtctactattgtgttaggggggtggactactggggccaaggcaccatggtcacggtgagcagc

SEQ ID NO: 406 - DNA, 338 bp, synthetic construct
  misc_feature 1..338: note = Description of Artificial Sequence: Synthetic polynucleotide
  source 1..338: mol_type = other DNA
gaggtgcagctcgtcgagtccggaggcggtctggtgcaacccggccgttctttgcggctgagttgcgctgccagtgggtatagcatcgtgagtcactattacatgcattgggttcgtcaagcccctggcaagggactagagtgggtgtccgccatctcctcagacggtagtaatgagtacgcggacagcgtagagggtagattcaccatttctcgggacaatgccaaaaatagttatacctccaaatgaattcccttagggccgaagacactgccgtgtactactgtgttcggggcgtggactactgggggcaggggacattggtgactgtgagctca

SEQ ID NO: 407 - DNA, 339 bp, synthetic construct
  misc_feature 1..339: note = Description of Artificial Sequence: Synthetic polynucleotide
  source 1..339: mol_type = other DNA
caggtccaactgcaggaatctggccccggactggttaaaccatctcagacactctccctgacctgcaccgtgtctggatacagcatcgtttctcattattactggtcatggattaggcagcatcccggaaaagggcttgaatggattggctacatctcctccgacggctccaatgagtacaacccatcacttaaatctctggtcacgataagcgtagacacatctaaaaatcagttctcattaaagctcagctctgttacagctgccgacaccgctgtgtacttctgtgtgcgaggggttgactactgggggcagggcacaatggtgacagtgtcttcc

SEQ ID NO: 408 - DNA, 339 bp, synthetic construct
  misc_feature 1..339: note = Description of Artificial Sequence: Synthetic polynucleotide
  source 1..339: mol_type = other DNA
caggttcaactggttcagtccggagccgaggtcaaaaagcctggatcctctgtgaaggtgtcatgtaaggcttctggctacagcatcgtctcacattattacatatcttgggtgcgacaggcccccggccaggggctcgagtggatgggaggtatttcctccgacgggagtaacaattacgctcagaaatttcagggccgggtgaccattaccgccgacgaaagtacaagcaccgcttatatggaattaagctctttaagatcagaggacacggctgtgtactactgtgtaaggggcgtggattactggggtcaggggacgctcgtcaccgtctcgagc

SEQ ID NO: 409 - DNA, 339 bp, synthetic construct
  misc_feature 1..339: note = Description of Artificial Sequence: Synthetic polynucleotide
  source 1..339: mol_type = other DNA
caggtccagctccaggaatccggcccagggttggtgaagccttcggagaccctgtctctgacatgcacagtcagcggctatagtatcgtctcccactattattggtcttggattcggcaacctccaggcaaggggttagaatggattggatacatctcaagcgatgggtccaataactacaacccaagtctcaaaagtagagtgactatctctgtggataccagtaaaaaccagttttcactcaagttgagttccgtcaccgccgccgacacagccgtttactactgtgttcggggagtggactactggggccaaggtaccacggttaccgtgagcagc

SEQ ID NO: 410 - DNA, 339 bp, synthetic construct
  misc_feature 1..339: note = Description of Artificial Sequence: Synthetic polynucleotide
  source 1..339: mol_type = other DNA
caggtgcagctgcaggagagcggccccggcctggtgaagcccagcgacaccctgagcctgacctgcgccgtgagcggctacagcatcgtgagccactactactggcactggatcagacagccccccggcaagggcctggagtggatgggctacatcagcagcgacggcagcaacgacttcaaccccagcctgaagaccagaatcaccatcagcagagacaccagcaagaaccagttcagcctgaagctgagcagcgtgaccgccgtggacaccgccgtgtactactgcgtgagaggcgtggactactggggccagggcaccctggtgaccgtgagcagc

SEQ ID NO: 411 - DNA, 339 bp, synthetic construct
  misc_feature 1..339: note = Description of Artificial Sequence: Synthetic polynucleotide
  source 1..339: mol_type = other DNA
caggtgcagctgcaggagagcggccccggcctggtgaagcccagccagaccctgagcctgacctgcgccgtgtacggctacagcatcgtgagccactactactggagctggatcagacagccccccggcaagggcctggagtggatcggcgagatcagcagcgacggcagcaacaactacaaccccagcctgaagagcagagtgaccatcagcgtggacaccagcaagaaccagttcagcctgaagctgagcagcgtgaccgccgccgacaccgccgtgtactactgcgtgagaggcgtggactactggggccagggcaccctggtgaccgtgagcagc

SEQ ID NO: 412 - DNA, 339 bp, synthetic construct
  misc_feature 1..339: note = Description of Artificial Sequence: Synthetic polynucleotide
  source 1..339: mol_type = other DNA
caggtgcagctgcaggagagcggccccggcctggtgaagcccagcgagaccctgagcctgacctgcgccgtgagcggctacagcatcgtgagccactactactggggctggatcagacagccccccggcaagggcctggagtggatcggcagcatcagcagcgacggcagcaactactacaaccccagcctgaagagcagagtgaccatcagcgtggacaccagcaagaaccagttcagcctgaagctgagcagcgtgaccgccgccgacaccgccgtgtactactgcgtgagaggcgtggactactggggccagggcaccctggtgaccgtgagcagc

SEQ ID NO: 413 - DNA, 339 bp, synthetic construct
  misc_feature 1..339: note = Description of Artificial Sequence: Synthetic polynucleotide
  source 1..339: mol_type = other DNA
caggtgcagctggtggagagcggcggcggcgtggtgcagcccggcagaagcctgagactgagctgcgccgccagcggctacagcatcgtgagccactactactggaactgggtgagacaggcccccggcaagggcctggagtgggtggcctacatcagcagcgacggcagcaacgagtacaaccccagcctgaagaacagattcaccatcagcagagacaacagcaagaacaccctgtacctgcagatgaacagcctgagagccgaggacaccgccgtgtactactgcgtgagaggcgtggactactggggccagggcaccaccgtgaccgtgagcagc

SEQ ID NO: 414 - DNA, 339 bp, synthetic construct
  misc_feature 1..339: note = Description of Artificial Sequence: Synthetic polynucleotide
  source 1..339: mol_type = other DNA
caggtgcagctgcaggagagcggccccggcctggtgaagcccagccagaccctgagcctgacctgcaccgtgagcggctacagcatcgtgagccactactactggaactggatcagacagcaccccggcaagggcctggagtggatcggctacatcagcagcgacggcagcaacgagtacaaccccagcctgaagaacctggtgaccatcagcgtggacaccagcaagaaccagttcagcctgaagctgagcagcgtgaccgccgccgacaccgccgtgtactactgcgtgagaggcgtggactactggggccagggcaccatggtgaccgtgagcagc

SEQ ID NO: 415 - DNA, 339 bp, synthetic construct
  misc_feature 1..339: note = Description of Artificial Sequence: Synthetic polynucleotide
  source 1..339: mol_type = other DNA
caggtgcagctgcaggagagcggccccggcctggtgaagcccagcgacaccctgagcctgacctgcgccgtgagcggctacagcatcgtgagccactactactggaactggatcagacagccccccggcaagggcctggagtggatcggctacatcagcagcgacggcagcaacgagtacaaccccagcctgaagaacagagtgaccatgagcgtggacaccagcaagaaccagttcagcctgaagctgagcagcgtgaccgccgtggacaccgccgtgtactactgcgtgagaggcgtggactactggggccagggcaccatggtgaccgtgagcagc

SEQ ID NO: 416 - DNA, 339 bp, synthetic construct
  misc_feature 1..339: note = Description of Artificial Sequence: Synthetic polynucleotide
  source 1..339: mol_type = other DNA
gaggtgcagctgctggagagcggcggcggcctggtgcagcccggcggcagcctgagactgagctgcgccgccagcggctacagcatcgtgagccactactactggaactgggtgagacaggcccccggcaagggcctggagtgggtgagctacatcagcagcgacggcagcaacgagtacaaccccagcctgaagaacagattcaccatcagcagagacaacagcaagaacaccctgtacctgcagatgaacagcctgagagccgaggacaccgccgtgtactactgcgtgagaggcgtggactactggggccagggcaccctggtgaccgtgagcagc

SEQ ID NO: 417 - protein, 106 aa, synthetic construct
  REGION 1..106: note = Description of Artificial Sequence: Synthetic polypeptide
DIQMTQSPSSLSASVGDRVTITCQASRDINNFLNWYQQKPGKAPKLLIYRANNLETGVPSRFSGSGSGTDFTFTISSLQPEDIATYFCLQYGDLYTFGGGTKVEIK

SEQ ID NO: 418 - protein, 106 aa, synthetic construct
  REGION 1..106: note = Description of Artificial Sequence: Synthetic polypeptide
DIQMTQSPSSLSASVGDRVTITCQASRDINNFLNWYQQKPGKAPKLLIYRANNLETGVPSRFSGSGSGTDFTFTISSLQPEDIATYYCLQYGDLYTFGGGTKVEIK

SEQ ID NO: 419 - protein, 106 aa, synthetic construct
  REGION 1..106: note = Description of Artificial Sequence: Synthetic polypeptide
DIQMTQSPSSLSASVGDRVTITCRASRDINNFLGWYQQKPGKAPKRLIYRANSLQSGVPSRFSGSGSGTEFTLTISSLQPEDFATYYCLQYGDLYTFGQGTKVEIK

SEQ ID NO: 420 - protein, 106 aa, synthetic construct
  REGION 1..106: note = Description of Artificial Sequence: Synthetic polypeptide
EIVLTQSPGTLSLSPGERATLSCRASRDINNFLAWYQQKPGQAPRLLIYRANSRATGIPDRFSGSGSGTDFTLTISRLEPEDFAVYYCLQYGDLYTFGQGTKVEIK

SEQ ID NO: 421 - protein, 106 aa, synthetic construct
  REGION 1..106: note = Description of Artificial Sequence: Synthetic polypeptide
EIVLTQSPATLSLSPGERATLSCRASRDINNFLAWYQQKPGQAPRLLIYRANNRATGIPARFSGSGPGTDFTLTISSLEPEDFAVYYCLQYGDLYTFGGGTKVEIK

SEQ ID NO: 422 - protein, 106 aa, synthetic construct
  REGION 1..106: note = Description of Artificial Sequence: Synthetic polypeptide
EIVLTQSPDFQSVTPKEKVTITCRASRDINNFLHWYQQKPDQSPKLLIKRANQSFSGVPSRFSGSGSGTDFTLTINSLEAEDAATYYCLQYGDLYTFGQGTKVEIK

SEQ ID NO: 423 - protein, 106 aa, synthetic construct
  REGION 1..106: note = Description of Artificial Sequence: Synthetic polypeptide
DIQMTQSPSSLSASVGDRVTITCRASRDINNFLAWFQQKPGKAPKSLIYRANSLQSGVPSRFSGSGSGTDFTLTISSLQPEDFATYYCLQYGDLYTFGGGTKVEIK

SEQ ID NO: 424 - protein, 106 aa, synthetic construct
  REGION 1..106: note = Description of Artificial Sequence: Synthetic polypeptide
DIQMTQSPSSLSASVGDRVTITCRASRDINNFLAWYQQKPGKAPKLLLYRANRLESGVPSRFSGSGSGTDYTLTISSLQPEDFATYYCLQYGDLYTFGGGTKVEIK

SEQ ID NO: 425 - protein, 106 aa, synthetic construct
  REGION 1..106: note = Description of Artificial Sequence: Synthetic polypeptide
DIQMTQSPSSLSASVGDRVTITCKASRDINNFLSWYQQKPGKAPKLLIYRANRLVDGVPSRFSGSGSGTDFTFTISSLQPEDIATYYCLQYGDLYTFGGGTKVEIK

SEQ ID NO: 426 - protein, 106 aa, synthetic construct
  REGION 1..106: note = Description of Artificial Sequence: Synthetic polypeptide
DIQMTQSPSSLSASVGDRVTITCKASRDINNFLSWYQQKPGKAPKLLIYRANRLVDGVPSRFSGSGSGTDFTLTISSLQPEDFATYYCLQYGDLYTFGQGTKVEIK

SEQ ID NO: 427 - protein, 106 aa, synthetic construct
  REGION 1..106: note = Description of Artificial Sequence: Synthetic polypeptide
EIVLTQSPGTLSLSPGERATLSCKASRDINNFLSWYQQKPGQAPRLLIYRANRLVDGIPDRFSGSGSGTDFTLTISRLEPEDFAVYYCLQYGDLYTFGQGTKVEIK

SEQ ID NO: 428 - protein, 106 aa, synthetic construct
  REGION 1..106: note = Description of Artificial Sequence: Synthetic polypeptide
EIVLTQSPATLSLSPGERATLSCKASRDINNFLSWYQQKPGQAPRLLIYRANRLVDGIPARFSGSGSGTDFTLTISSLEPEDFAVYYCLQYGDLYTFGQGTKVEIK

SEQ ID NO: 429 - DNA, 318 bp, synthetic construct
  misc_feature 1..318: note = Description of Artificial Sequence: Synthetic polynucleotide
  source 1..318: mol_type = other DNA
gacatacagatgactcagagcccctcctcactctcggcatcagtcggcgacagggtcacaattacctgtcaggcttctcgcgacattaataacttcctgaattggtatcagcaaaagcccgggaaggcccctaagctgttgatttatagagcaaataatctcgaaaccggcgtgcccagtaggtttagcgggtccgggagcggaacagacttcacattcaccatttctagtttgcagcccgaagacattgctacatatttttgcctgcagtacggggatctctacactttcgggggcggaacaaaggttgagataaaa

SEQ ID NO: 430 - DNA, 318 bp, synthetic construct
  misc_feature 1..318: note = Description of Artificial Sequence: Synthetic polynucleotide
  source 1..318: mol_type = other DNA
gatattcaaatgacgcagtcaccctcatcgctctctgcgtcagtaggggatcgtgtcacgataacctgtcaagcatcaagggacatcaacaacttcctcaactggtaccaacagaagcctggcaaggcacctaaactcctgatctaccgggctaacaacctagaaaccggggttccgagccgattcagtgggtctggaagcgggacagactttacgttcactattagttcgctacagcccgaagacattgcgacatattactgtcttcagtatggggatttgtatacctttgggggaggcaccaaggtagagataaag

SEQ ID NO: 431 - DNA, 318 bp, synthetic construct
  misc_feature 1..318: note = Description of Artificial Sequence: Synthetic polynucleotide
  source 1..318: mol_type = other DNA
gacatccagatgactcagagcccgtcttctctatccgcaagtgtaggcgatcgtgtcaccatcacatgccgggcttcccgggatatcaacaacttccttgggtggtatcagcagaagcccggaaaagcccccaaacggctcatctacagagcgaattccctgcagtcaggtgtccccagtaggttcagcggatcaggctcggggaccgaattcactctgaccattagctcactgcagcctgaggatttcgctacttactattgcctgcaatacggcgatctgtacactttcgggcagggcaccaaggtggaaataaaa

SEQ ID NO: 432 - DNA, 318 bp, synthetic construct
  misc_feature 1..318: note = Description of Artificial Sequence: Synthetic polynucleotide
  source 1..318: mol_type = other DNA
gaaatcgtactgacccagtctcccggaaccctgagtctctcacccggcgagcgcgcaacactgtcgtgtagggccagtagggacataaataacttcctagcctggtaccaacaaaaaccgggtcaggctccaagactgttgatctatagagctaactccagggccaccggcatcccagaccgattctcaggctccggatctggaaccgacttcacgctcaccattagccgactagaacctgaggactttgctgtatactattgcctgcagtacggcgacctgtatacctttggacagggtaccaaggtcgagatcaag

SEQ ID NO: 433 - DNA, 318 bp, synthetic construct
  misc_feature 1..318: note = Description of Artificial Sequence: Synthetic polynucleotide
  source 1..318: mol_type = other DNA
gagatcgtacttacgcagagcccagcaactctgtctctgtcccccggagaacgggccaccctgtcgtgccgggccagccgtgatattaataatttcctggcctggtatcaacaaaaaccggggcaggctcctcgactgttgatctaccgggccaacaatagagcaactggtatccctgctcgcttctccggcagtgggccaggtacagacttcaccctgactatttcgtcactcgaaccagaagactttgccgtgtattattgcttacaatacggggatctgtacactttcggaggaggaactaaggtcgaaattaag

SEQ ID NO: 434 - DNA, 318 bp, synthetic construct
  misc_feature 1..318: note = Description of Artificial Sequence: Synthetic polynucleotide
  source 1..318: mol_type = other DNA
gagatcgtgctgacccagagccccgacttccagagcgtgacccccaaggagaaggtgaccatcacctgcagagccagcagagacatcaacaacttcctgcactggtaccagcagaagcccgaccagagccccaagctgctgatcaagagagccaaccagagcttcagcggcgtgcccagcagattcagcggcagcggcagcggcaccgacttcaccctgaccatcaacagcctggaggccgaggacgccgccacctactactgcctgcagtacggcgacctgtacaccttcggccagggcaccaaggtggagatcaag

SEQ ID NO: 435 - DNA, 318 bp, synthetic construct
  misc_feature 1..318: note = Description of Artificial Sequence: Synthetic polynucleotide
  source 1..318: mol_type = other DNA
gacatccagatgacccagagccccagcagcctgagcgccagcgtgggcgacagagtgaccatcacctgcagagccagcagagacatcaacaacttcctggcctggttccagcagaagcccggcaaggcccccaagagcctgatctacagagccaacagcctgcagagcggcgtgcccagcagattcagcggcagcggcagcggcaccgacttcaccctgaccatcagcagcctgcagcccgaggacttcgccacctactactgcctgcagtacggcgacctgtacaccttcggcggcggcaccaaggtggagatcaag

SEQ ID NO: 436 - DNA, 318 bp, synthetic construct
  misc_feature 1..318: note = Description of Artificial Sequence: Synthetic polynucleotide
  source 1..318: mol_type = other DNA
gacatccagatgacccagagccccagcagcctgagcgccagcgtgggcgacagagtgaccatcacctgcagagccagcagagacatcaacaacttcctggcctggtaccagcagaagcccggcaaggcccccaagctgctgctgtacagagccaacagactggagagcggcgtgcccagcagattcagcggcagcggcagcggcaccgactacaccctgaccatcagcagcctgcagcccgaggacttcgccacctactactgcctgcagtacggcgacctgtacaccttcggcggcggcaccaaggtggagatcaag

SEQ ID NO: 437 - DNA, 318 bp, synthetic construct
  misc_feature 1..318: note = Description of Artificial Sequence: Synthetic polynucleotide
  source 1..318: mol_type = other DNA
gacatccagatgacccagagccccagcagcctgagcgccagcgtgggcgacagagtgaccatcacctgcaaggccagcagagacatcaacaacttcctgagctggtaccagcagaagcccggcaaggcccccaagctgctgatctacagagccaacagactggtggacggcgtgcccagcagattcagcggcagcggcagcggcaccgacttcaccttcaccatcagcagcctgcagcccgaggacatcgccacctactactgcctgcagtacggcgacctgtacaccttcggcggcggcaccaaggtggagatcaag

SEQ ID NO: 438 - DNA, 318 bp, synthetic construct
  misc_feature 1..318: note = Description of Artificial Sequence: Synthetic polynucleotide
  source 1..318: mol_type = other DNA
gacatccagatgacccagagccccagcagcctgagcgccagcgtgggcgacagagtgaccatcacctgcaaggccagcagagacatcaacaacttcctgagctggtaccagcagaagcccggcaaggcccccaagctgctgatctacagagccaacagactggtggacggcgtgcccagcagattcagcggcagcggcagcggcaccgacttcaccctgaccatcagcagcctgcagcccgaggacttcgccacctactactgcctgcagtacggcgacctgtacaccttcggccagggcaccaaggtggagatcaag

SEQ ID NO: 439 - DNA, 318 bp, synthetic construct
  misc_feature 1..318: note = Description of Artificial Sequence: Synthetic polynucleotide
  source 1..318: mol_type = other DNA
gagatcgtgctgacccagagccccggcaccctgagcctgagccccggcgagagagccaccctgagctgcaaggccagcagagacatcaacaacttcctgagctggtaccagcagaagcccggccaggcccccagactgctgatctacagagccaacagactggtggacggcatccccgacagattcagcggcagcggcagcggcaccgacttcaccctgaccatcagcagactggagcccgaggacttcgccgtgtactactgcctgcagtacggcgacctgtacaccttcggccagggcaccaaggtggagatcaag

SEQ ID NO: 440 - DNA, 318 bp, synthetic construct
  misc_feature 1..318: note = Description of Artificial Sequence: Synthetic polynucleotide
  source 1..318: mol_type = other DNA
gagatcgtgctgacccagagccccgccaccctgagcctgagccccggcgagagagccaccctgagctgcaaggccagcagagacatcaacaacttcctgagctggtaccagcagaagcccggccaggcccccagactgctgatctacagagccaacagactggtggacggcatccccgccagattcagcggcagcggcagcggcaccgacttcaccctgaccatcagcagcctggagcccgaggacttcgccgtgtactactgcctgcagtacggcgacctgtacaccttcggccagggcaccaaggtggagatcaag

SEQ ID NO: 441 - protein, 589 aa, synthetic construct
  REGION 1..589: note = Description of Artificial Sequence: Synthetic polypeptide
QVQLQESGPGLVKPSETLSLTCAVSGYSIVSHYYWGWIRQPPGKGLEWIGSISSDGSNYYNPSLKSRVTISVDTSKNQFSLKLSSVTAADTAVYYCVRGVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKQGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 442 - DNA, 1769 bp, synthetic construct
  misc_feature 1..1769: note = Description of Artificial Sequence: Synthetic polynucleotide
  source 1..1769: mol_type = other DNA
caggtgcagctgcaggagagcggccccggcctggtgaagcccagcgagaccctgagcctgacctgcgccgtgagcggctacagcatcgtgagccactactactggggctggatcagacagccccccggcaagggcctggagtggatcggcagcatcagcagcgacggcagcaactactacaaccccagcctgaagagcagagtgaccatcagcgtggacaccagcaagaaccagttcagcctgaagctgagcagcgtgaccgccgccgacaccgccgtgtactactgcgtgagaggcgtggactactggggccagggcaccctggtgaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagtgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggaggtggttctggaggtggaggtagtatccctcctcacgtacagaagtccgtgaacaatgacatgattgtcactgacaataacggagccgtcaagtttcctcagctatgtaagttctgcgatgttcggttctccacatgcgataatcagaaaagctgtatgtctaattgcagtatcactagtatatgcgaaaaacctcaagaagtttgcgtcgccgtgtggcggaaaaatgatgaaaatatcacgcttgagactgtctgccatgatccaaagttaccctaccacgacttcatcttagaagacgccgcatcacccaagtgcattatgaaagagaaaaagaagccaggagaaacattctttatgtgctcatgctcctctgacgaatgcaacgacaacattatcttctctgaggagtataacacctcaaatccagactga

SEQ ID NO: 443 - protein, 212 aa, synthetic construct
  REGION 1..212: note = Description of Artificial Sequence: Synthetic polypeptide
DIQMTQSPSSLSASVGDRVTITCKASRDINNFLSWYQQKPGKAPKLLIYRANRLVDGVPSRFSGSGSGTDFTLTISSLQPEDFATYYCLQYGDLYTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 444 - protein, 119 aa, synthetic construct
  REGION 1..119: note = Description of Artificial Sequence: Synthetic polypeptide
QVQLVQSGAEVKKPGSSVKVSCKASGYAFSNFWMNWVRQAPGQGLEWMGQIYPGDGDTNYNGKFKGRVTITADKSTSTAYMELSSLRSEDTAVYYCARSYYRSAWFAYWGQGTLVTVSS

SEQ ID NO: 445 - protein, 119 aa, synthetic construct
  REGION 1..119: note = Description of Artificial Sequence: Synthetic polypeptide
QVQLVQSGAEVKKPGASVKVSCKASGYAFSNFWMNWVRQAPGQGLEWMGQIYPGDGDTNYNGKFKGRVTMTRDTSTSTVYMELSSLRSEDTAVYYCARSYYRSAWFAYWGQGTLVTVSS

SEQ ID NO: 446 - protein, 119 aa, synthetic construct
  REGION 1..119: note = Description of Artificial Sequence: Synthetic polypeptide
QVQLVQSGAEVKKPGASVKVSCKASGYAFSNFWMNWVRQAPGQGLEWMGQIYPGDGDTNYNGKFKGRVTMTRDTSISTAYMELSRLRSDDTAVYYCARSYYRSAWFAYWGQGTLVTVSS

SEQ ID NO: 447 - protein, 119 aa, synthetic construct
  REGION 1..119: note = Description of Artificial Sequence: Synthetic polypeptide
QVQLVQSGAEVKKPGATVKISCKVSGYAFSNFWMNWVQQAPGKGLEWMGQIYPGDGDTNYNGKFKGRVTITADTSTDTAYMELSSLRSEDTAVYYCARSYYRSAWFAYWGQGTLVTVSR

SEQ ID NO: 448 - protein, 119 aa, synthetic construct
  REGION 1..119: note = Description of Artificial Sequence: Synthetic polypeptide
EVQLVQSGAEVKKPGESLKISCKGSGYAFSNFWMNWVRQMPGKGLEWMGQIYPGDGDTNYNGKFKGQVTISADKSISTAYLQWSSLKASDTAMYYCARSYYRSAWFAYWGQGTLVTVSL

SEQ ID NO: 449 - protein, 107 aa, synthetic construct
  REGION 1..107: note = Description of Artificial Sequence: Synthetic polypeptide
EIVLTQSPDFQSVTPKEKVTITCRASQSIGTSIHWYQQKPDQSPKLLIKYASESISGVPSRFSGSGSGTDFTLTINSLEAEDAATYYCQQSNNWPLTFGQGTKVEIK

SEQ ID NO: 450 - protein, 107 aa, synthetic construct
  REGION 1..107: note = Description of Artificial Sequence: Synthetic polypeptide
EIVMTQSPATLSVSPGERATLSCRASQSIGTSIHWYQQKPGQAPRLLIYYASESISGIPARFSGSGSGTEFTLTISSLQSEDFAVYYCQQSNNWPLTFGGGTKVEIK

SEQ ID NO: 451 - protein, 107 aa, synthetic construct
  REGION 1..107: note = Description of Artificial Sequence: Synthetic polypeptide
EIVLTQSPATLSLSPGERATLSCRASQSIGTSIHWYQQKPGQAPRLLIYYASESISGIPARFSGSGSGTDFTLTISSLEPEDFAVYYCQQSNNWPLTFGGGTKVEIK

SEQ ID NO: 452 - protein, 107 aa, synthetic construct
  REGION 1..107: note = Description of Artificial Sequence: Synthetic polypeptide
AIQLTQSPSSLSASVGDRVTITCRASQSIGTSIHWYQQKPGKAPKLLIYYASESISGVPSRFSGSGSGTDFTLTISSLQPEDFATYYCQQSNNWPLTFGGGTKVEIK

SEQ ID NO: 453 - protein, 107 aa, synthetic construct
  REGION 1..107: note = Description of Artificial Sequence: Synthetic polypeptide
DIVMTQSPSLLSASTGDRVTISCRASQSIGTSIHWYQQKPGKAPELLIYYASESISGVPSRFSGSGSGTDFTLTISCLQSEDFATYYCQQSNNWPLTFGQGTKVEIK

SEQ ID NO: 454 - protein, 200 aa, synthetic construct
  REGION 1..200: note = Description of Artificial Sequence: Synthetic polypeptide
QVQLQQSGAELVRPGSSVKISCKTSGYAFSNFWMNWVKQRPGQGLEWIGQIYPGDGDTNYNGKFKGKATLTADKSSSTAYMQLSSLTSEASAVYFCARSYYRSAWFAYWGQGTLVSVSAASTKGPSVFPLAPSSKSTSGGTAALGCLVKDYFPEPVTVSWNSGALTSGVHTFPAVLQSSGLYSLSSVVTVPSSSLGTQTY

SEQ ID NO: 455 - protein, 155 aa, synthetic construct
  REGION 1..155: note = Description of Artificial Sequence: Synthetic polypeptide
DILLTQSPAILSVSPGERVSFSCRASQSIGTSIHWYQQRTNGSPRLLIKYASESISGIPSRFSGSGSGTDFTLSINSVESEDIADYYCQQSNNWPLTFGAGTKLELKRTVAAPSVFIFPPSDEQLKSGTASVVCLLNNFYPREAKVQWKVDNALQ

SEQ ID NO: 456 - protein, 127 aa, synthetic construct
  REGION 1..127: note = Description of Artificial Sequence: Synthetic polypeptide
QVQLVQSGAEVKKPGSSVKVSCKASGYAFSNFWMNWVRQAPGQGLEWMGQIYPGDGDTNYNGKFKGRVTITADKSTSTAYMELSSLRSEDTAVYYCARSYYRSAWFAYWGQGTLVTVSSASTKGPSV

SEQ ID NO: 457 - protein, 127 aa, synthetic construct
  REGION 1..127: note = Description of Artificial Sequence: Synthetic polypeptide
QVQLVQSGAEVKKPGASVKVSCKASGYAFSNFWMNWVRQAPGQGLEWMGQIYPGDGDTNYNGKFKGRVTMTRDTSTSTVYMELSSLRSEDTAVYYCARSYYRSAWFAYWGQGTLVTVSSASTKGPSV

SEQ ID NO: 458 - protein, 127 aa, synthetic construct
  REGION 1..127: note = Description of Artificial Sequence: Synthetic polypeptide
QVQLVQSGAEVKKPGASVKVSCKASGYAFSNFWMNWVRQAPGQGLEWMGQIYPGDGDTNYNGKFKGRVTMTRDTSISTAYMELSRLRSDDTAVYYCARSYYRSAWFAYWGQGTLVTVSSASTKGPSV

SEQ ID NO: 459 - protein, 127 aa, synthetic construct
  REGION 1..127: note = Description of Artificial Sequence: Synthetic polypeptide
QVQLVQSGAEVKKPGATVKISCKVSGYAFSNFWMNWVQQAPGKGLEWMGQIYPGDGDTNYNGKFKGRVTITADTSTDTAYMELSSLRSEDTAVYYCARSYYRSAWFAYWGQGTLVTVSRASTKGPSV

SEQ ID NO: 460 - protein, 127 aa, synthetic construct
  REGION 1..127: note = Description of Artificial Sequence: Synthetic polypeptide
EVQLVQSGAEVKKPGESLKISCKGSGYAFSNFWMNWVRQMPGKGLEWMGQIYPGDGDTNYNGKFKGQVTISADKSISTAYLQWSSLKASDTAMYYCARSYYRSAWFAYWGQGTLVTVSLASTKGPSV

SEQ ID NO: 461 - protein, 126 aa, synthetic construct
  REGION 1..126: note = Description of Artificial Sequence: Synthetic polypeptide
EIVLTQSPDFQSVTPKEKVTITCRASQSIGTSIHWYQQKPDQSPKLLIKYASESISGVPSRFSGSGSGTDFTLTINSLEAEDAATYYCQQSNWPLTFGQGTKVEIKRTVAAPSVFIFPPSDEQLKS

SEQ ID NO: 462 - protein, 127 aa, synthetic construct
  REGION 1..127: note = Description of Artificial Sequence: Synthetic polypeptide
EIVMTQSPATLSVSPGERATLSCRASQSIGTSIHWYQQKPGQAPRLLIYYASESISGIPARFSGSGSGTEFTLTISSLQSEDFAVYYCQQSNNWPLTFGGGTKVEIKRTVAAPSVFIFPPSDEQLKS

SEQ ID NO: 463 - protein, 127 aa, synthetic construct
  REGION 1..127: note = Description of Artificial Sequence: Synthetic polypeptide
EIVLTQSPATLSLSPGERATLSCRASQSIGTSIHWYQQKPGQAPRLLIYYASESISGIPARFSGSGSGTDFTLTISSLEPEDFAVYYCQQSNNWPLTFGGGTKVEIKRTVAAPSVFIFPPSDEQLKS

SEQ ID NO: 464 - protein, 127 aa, synthetic construct
  REGION 1..127: note = Description of Artificial Sequence: Synthetic polypeptide
AIQLTQSPSSLSASVGDRVTITCRASQSIGTSIHWYQQKPGKAPKLLIYYASESISGVPSRFSGSGSGTDFTLTISSLQPEDFATYYCQQSNNWPLTFGGGTKVEIKRTVAAPSVFIFPPSDEQLKS

SEQ ID NO: 465 - protein, 127 aa, synthetic construct
  REGION 1..127: note = Description of Artificial Sequence: Synthetic polypeptide
DIVMTQSPSLLSASTGDRVTISCRASQSIGTSIHWYQQKPGKAPELLIYYASESISGVPSRFSGSGSGTDFTLTISCLQSEDFATYYCQQSNNWPLTFGQGTKVEIKRTVAAPSVFIFPPSDEQLKS

SEQ ID NO: 466 - protein, 595 aa, synthetic construct
  REGION 1..595: note = Description of Artificial Sequence: Synthetic polypeptide
QVQLVQSGAEVKKPGSSVKVSCKASGYAFSNFWMNWVRQAPGQGLEWMGQIYPGDGDTNYNGKFKGRVTITADKSTSTAYMELSSLRSEDTAVYYCARSYYRSAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 467 - protein, 213 aa, synthetic construct
  REGION 1..213: note = Description of Artificial Sequence: Synthetic polypeptide
EIVLTQSPDFQSVTPKEKVTITCRASQSIGTSIHWYQQKPDQSPKLLIKYASESISGVPSRFSGSGSGTDFTLTINSLEAEDAATYYCQQSNNWPLTFGQGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 468 - protein, 14 aa, synthetic construct
  REGION 1..14: note = Description of Artificial Sequence: Synthetic peptide
RIWFIPRSSWYERA

SEQ ID NO: 469 - protein, 13 aa, synthetic construct
  REGION 1..13: note = Description of Artificial Sequence: Synthetic peptide
IWFIPRSSWYERA

SEQ ID NO: 470 - protein, 11 aa, synthetic construct
  REGION 1..11: note = Description of Artificial Sequence: Synthetic peptide
FIPRSSWYERA

SEQ ID NO: 471 - protein, 11 aa, synthetic construct
  REGION 1..11: note = Description of Artificial Sequence: Synthetic peptide
FIPRSSWYERA

SEQ ID NO: 472 - protein, 10 aa, synthetic construct
  REGION 1..10: note = Description of Artificial Sequence: Synthetic peptide
IPRSSWYERA

SEQ ID NO: 473 - protein, 9 aa, synthetic construct
  REGION 1..9: note = Description of Artificial Sequence: Synthetic peptide
PRSSWYERA

SEQ ID NO: 474 - protein, 14 aa, synthetic construct
  REGION 1..14: note = Description of Artificial Sequence: Synthetic peptide
KRIWFIPRSSWYER

SEQ ID NO: 475 - protein, 13 aa, synthetic construct
  REGION 1..13: note = Description of Artificial Sequence: Synthetic peptide
KRIWFIPRSSWYE

SEQ ID NO: 476 - protein, 12 aa, synthetic construct
  REGION 1..12: note = Description of Artificial Sequence: Synthetic peptide
KRIWFIPRSSWY

SEQ ID NO: 477 - protein, 11 aa, synthetic construct
  REGION 1..11: note = Description of Artificial Sequence: Synthetic peptide
KRIWFIPRSSW

SEQ ID NO: 478 - protein, 10 aa, synthetic construct
  REGION 1..10: note = Description of Artificial Sequence: Synthetic peptide
KRIWFIPRSS

SEQ ID NO: 479 - protein, 9 aa, synthetic construct
  REGION 1..9: note = Description of Artificial Sequence: Synthetic peptide
KRIWFIPRS

SEQ ID NO: 480 - protein, 13 aa, synthetic construct
  REGION 1..13: note = Description of Artificial Sequence: Synthetic peptide
RIWFIPRSSWYER

SEQ ID NO: 481 - protein, 11 aa, synthetic construct
  REGION 1..11: note = Description of Artificial Sequence: Synthetic peptide
IWFIPRSSWYE

SEQ ID NO: 482 - protein, 9 aa, synthetic construct
  REGION 1..9: note = Description of Artificial Sequence: Synthetic peptide
WFIPRSSWY

SEQ ID NO: 483 - protein, 15 aa, synthetic construct
  REGION 1..15: note = Description of Artificial Sequence: Synthetic peptide
DRRIFWWSLRSAPGA

SEQ ID NO: 484 - protein, 15 aa, synthetic construct
  REGION 1..15: note = Description of Artificial Sequence: Synthetic peptide
HVRLHHYLRHRSLPN

SEQ ID NO: 485 - protein, 15 aa, synthetic construct
  REGION 1..15: note = Description of Artificial Sequence: Synthetic peptide
RFFTRFPWHYHASRL

SEQ ID NO: 486 - protein, 15 aa, synthetic construct
  REGION 1..15: note = Description of Artificial Sequence: Synthetic peptide
RLAHSHRHRSHVALT

SEQ ID NO: 487 - protein, 15 aa, synthetic construct
  REGION 1..15: note = Description of Artificial Sequence: Synthetic peptide
RRWVRYPVHLHSPIV

SEQ ID NO: 488 - protein, 15 aa, synthetic construct
  REGION 1..15: note = Description of Artificial Sequence: Synthetic peptide
PPYHRFWRGHRHAVQ

SEQ ID NO: 489 - protein, 15 aa, synthetic construct
  REGION 1..15: note = Description of Artificial Sequence: Synthetic peptide
HRISHFAHRYLARLH

SEQ ID NO: 490 - protein, 15 aa, synthetic construct
  REGION 1..15: note = Description of Artificial Sequence: Synthetic peptide
WHWRHRIPLQLAAGR

SEQ ID NO: 491 - protein, 15 aa, synthetic construct
  REGION 1..15: note = Description of Artificial Sequence: Synthetic peptide
GWHSLLHSRYHRIAA

SEQ ID NO: 492 - protein, 15 aa, synthetic construct
  REGION 1..15: note = Description of Artificial Sequence: Synthetic peptide
FVWVRFHRLPRQIYT

SEQ ID NO: 493 - protein, 15 aa, synthetic construct
  REGION 1..15: note = Description of Artificial Sequence: Synthetic peptide
WHKYFLRRPLSVRTR

SEQ ID NO: 494 - protein, 15 aa, synthetic construct
  REGION 1..15: note = Description of Artificial Sequence: Synthetic peptide
VPMALNHGVYVMVSS

SEQ ID NO: 495 - protein, 15 aa, synthetic construct
  REGION 1..15: note = Description of Artificial Sequence: Synthetic peptide
RKWFLQHRRMPVSVL

SEQ ID NO: 496 - protein, 15 aa, synthetic construct
  REGION 1..15: note = Description of Artificial Sequence: Synthetic peptide
SGRRHLHRHHIFSLP

SEQ ID NO: 497 - protein, 15 aa, synthetic construct
  REGION 1..15: note = Description of Artificial Sequence: Synthetic peptide
GWITFHRRHHDRVLS

SEQ ID NO: 498 - protein, 15 aa, synthetic construct
  REGION 1..15: note = Description of Artificial Sequence: Synthetic peptide
RLHGHRSHRFTHVAQ

SEQ ID NO: 499 - protein, 15 aa, synthetic construct
  REGION 1..15: note = Description of Artificial Sequence: Synthetic peptide
KRIWFIPRSSWYERA

SEQ ID NO: 500 - protein, 15 aa, synthetic construct
  REGION 1..15: note = Description of Artificial Sequence: Synthetic peptide
MPLSRYWWLFSHRPR

SEQ ID NO: 501 - protein, 15 aa, synthetic construct
  REGION 1..15: note = Description of Artificial Sequence: Synthetic peptide
RHLSHFKWLRSHGLD

SEQ ID NO: 502 - protein, 15 aa, synthetic construct
  REGION 1..15: note = Description of Artificial Sequence: Synthetic peptide
RRFHFHSRMVAVDNS

SEQ ID NO: 503 - protein, 16 aa, synthetic construct
  REGION 1..16: note = Description of Artificial Sequence: Synthetic peptide
HANFCLGPCPYIWSLA

SEQ ID NO: 504 - protein, 15 aa, synthetic construct
  REGION 1..15: note = Description of Artificial Sequence: Synthetic peptide
FCLGPCPYIWSLDTA

SEQ ID NO: 505 - protein, 16 aa, synthetic construct
  REGION 1..16: note = Description of Artificial Sequence: Synthetic peptide
SNPYSAFQVDIIVDIA

SEQ ID NO: 506 - protein, 13 aa, synthetic construct
  REGION 1..13: note = Description of Artificial Sequence: Synthetic peptide
TSLDATMIWTMMA

SEQ ID NO: 507 - protein, 14 aa, synthetic construct
  REGION 1..14: note = Description of Artificial Sequence: Synthetic peptide
TSLDASIWAMMQNA

SEQ ID NO: 508 - DNA, 42 bp, synthetic construct
  misc_feature 1..42: note = Description of Artificial Sequence: Synthetic oligonucleotide
  source 1..42: mol_type = other DNA
aggatctggttcatccccaggagcagctggtacgagagggcc

SEQ ID NO: 509 - DNA, 39 bp, synthetic construct
  misc_feature 1..39: note = Description of Artificial Sequence: Synthetic oligonucleotide
  source 1..39: mol_type = other DNA
atctggttcatccccaggagcagctggtacgagagggcc

SEQ ID NO: 510 - DNA, 33 bp, synthetic construct
  misc_feature 1..33: note = Description of Artificial Sequence: Synthetic oligonucleotide
  source 1..33: mol_type = other DNA
ttcatccccaggagcagctggtacgagagggcc

SEQ ID NO: 511 - DNA, 33 bp, synthetic construct; identical to SEQ ID NO: 510
  misc_feature 1..33: note = Description of Artificial Sequence: Synthetic oligonucleotide
  source 1..33: mol_type = other DNA

SEQ ID NO: 512 - DNA, 30 bp, synthetic construct
  misc_feature 1..30: note = Description of Artificial Sequence: Synthetic oligonucleotide
  source 1..30: mol_type = other DNA
atccccaggagcagctggtacgagagggcc

SEQ ID NO: 513 - DNA, 27 bp, synthetic construct
  misc_feature 1..27: note = Description of Artificial Sequence: Synthetic oligonucleotide
  source 1..27: mol_type = other DNA
cccaggagcagctggtacgagagggcc

SEQ ID NO: 514 - DNA, 42 bp, synthetic construct
  misc_feature 1..42: note = Description of Artificial Sequence: Synthetic oligonucleotide
  source 1..42: mol_type = other DNA
aagaggatctggttcatccccaggagcagctggtacgagagg

SEQ ID NO: 515 - DNA, 39 bp, synthetic construct
  misc_feature 1..39: note = Description of Artificial Sequence: Synthetic oligonucleotide
  source 1..39: mol_type = other DNA
aagaggatctggttcatccccaggagcagctggtacgag

SEQ ID NO: 516 - DNA, 36 bp, synthetic construct
  misc_feature 1..36: note = Description of Artificial Sequence: Synthetic oligonucleotide
  source 1..36: mol_type = other DNA
aagaggatctggttcatccccaggagcagctggtac

SEQ ID NO: 517 - DNA, 33 bp, synthetic construct
  misc_feature 1..33: note = Description of Artificial Sequence: Synthetic oligonucleotide
  source 1..33: mol_type = other DNA
aagaggatctggttcatccccaggagcagctgg

SEQ ID NO: 518 - DNA, 30 bp, synthetic construct
  misc_feature 1..30: note = Description of Artificial Sequence: Synthetic oligonucleotide
  source 1..30: mol_type = other DNA
aagaggatctggttcatccccaggagcagc

SEQ ID NO: 519 - DNA, 27 bp, synthetic construct
  misc_feature 1..27: note = Description of Artificial Sequence: Synthetic oligonucleotide
  source 1..27: mol_type = other DNA
aagaggatctggttcatccccaggagc

SEQ ID NO: 520 - DNA, 39 bp, synthetic construct
  misc_feature 1..39: note = Description of Artificial Sequence: Synthetic oligonucleotide
  source 1..39: mol_type = other DNA
aggatctggttcatccccaggagcagctggtacgagagg

SEQ ID NO: 521 - DNA, 33 bp, synthetic construct
  misc_feature 1..33: note = Description of Artificial Sequence: Synthetic oligonucleotide
  source 1..33: mol_type = other DNA
atctggttcatccccaggagcagctggtacgag

SEQ ID NO: 522 - DNA, 27 bp, synthetic construct
  misc_feature 1..27: note = Description of Artificial Sequence: Synthetic oligonucleotide
  source 1..27: mol_type = other DNA
tggttcatccccaggagcagctggtac

SEQ ID NO: 523 - DNA, 45 bp, synthetic construct
  misc_feature 1..45: note = Description of Artificial Sequence: Synthetic oligonucleotide
  source 1..45: mol_type = other DNA
gacaggaggatcttctggtggagcctgaggagcgcccccggcgcc

SEQ ID NO: 524 - DNA, 45 bp, synthetic construct
  misc_feature 1..45: note = Description of Artificial Sequence: Synthetic oligonucleotide
  source 1..45: mol_type = other DNA
cacgtgaggctgcaccactacctgaggcacaggagcctgcccaac

SEQ ID NO: 525 - DNA, 45 bp, synthetic construct
  misc_feature 1..45: note = Description of Artificial Sequence: Synthetic oligonucleotide
  source 1..45: mol_type = other DNA
aggttcttcaccaggttcccctggcactaccacgccagcaggctg

SEQ ID NO: 526 - DNA, 45 bp, synthetic construct
  misc_feature 1..45: note = Description of Artificial Sequence: Synthetic oligonucleotide
  source 1..45: mol_type = other DNA
aggctggcccacagccacaggcacaggagccacgtggccctgacc

SEQ ID NO: 527 - DNA, 45 bp, synthetic construct
  misc_feature 1..45: note = Description of Artificial Sequence: Synthetic oligonucleotide
  source 1..45: mol_type = other DNA
aggaggtgggtgaggtaccccgtgcacctgcacagccccatcgtg

SEQ ID NO: 528 - DNA, 45 bp, synthetic construct
  misc_feature 1..45: note = Description of Artificial Sequence: Synthetic oligonucleotide
  source 1..45: mol_type = other DNA
cccccctaccacaggttctggaggggccacaggcacgccgtgcag

SEQ ID NO: 529 - DNA, 45 bp, synthetic construct
  misc_feature 1..45: note = Description of Artificial Sequence: Synthetic oligonucleotide
  source 1..45: mol_type = other DNA
cacaggatcagccacttcgcccacaggtacctggccaggctgcac

SEQ ID NO: 530 - DNA, 45 bp, synthetic construct
  misc_feature 1..45: note = Description of Artificial Sequence: Synthetic oligonucleotide
  source 1..45: mol_type = other DNA
tggcactggaggcacaggatccccctgcagctggccgccggcagg

SEQ ID NO: 531 - DNA, 45 bp, synthetic construct
  misc_feature 1..45: note = Description of Artificial Sequence: Synthetic oligonucleotide
  source 1..45: mol_type = other DNA
ggctggcacagcctgctgcacagcaggtaccacaggatcgccgcc

SEQ ID NO: 532 - DNA, 45 bp, synthetic construct
  misc_feature 1..45: note = Description of Artificial Sequence: Synthetic oligonucleotide
  source 1..45: mol_type = other DNA
ttcgtgtgggtgaggttccacaggctgcccaggcagatctacacc

SEQ ID NO: 533 - DNA, 45 bp, synthetic construct
  misc_feature 1..45: note = Description of Artificial Sequence: Synthetic oligonucleotide
  source 1..45: mol_type = other DNA
tggcacaagtacttcctgaggaggcccctgagcgtgaggaccagg

SEQ ID NO: 534 - DNA, 45 bp, synthetic construct
  misc_feature 1..45: note = Description of Artificial Sequence: Synthetic oligonucleotide
  source 1..45: mol_type = other DNA
gtgcccatggccctgaaccacggcgtgtacgtgatggtgagcagc

SEQ ID NO: 535 - DNA, 45 bp, synthetic construct
  misc_feature 1..45: note = Description of Artificial Sequence: Synthetic oligonucleotide
  source 1..45: mol_type = other DNA
aggaagtggttcctgcagcacaggaggatgcccgtgagcgtgctg

SEQ ID NO: 536 - DNA, 45 bp, synthetic construct
  misc_feature 1..45: note = Description of Artificial Sequence: Synthetic oligonucleotide
  source 1..45: mol_type = other DNA
agcggcaggaggcacctgcacaggcaccacatcttcagcctgccc

SEQ ID NO: 537 - DNA, 45 bp, synthetic construct
  misc_feature 1..45: note = Description of Artificial Sequence: Synthetic oligonucleotide
  source 1..45: mol_type = other DNA
ggctggatcaccttccacaggaggcaccacgacagggtgctgagc

SEQ ID NO: 538 - DNA, 45 bp, synthetic construct
  misc_feature 1..45: note = Description of Artificial Sequence: Synthetic oligonucleotide
  source 1..45: mol_type = other DNA
aggctgcacggccacaggagccacaggttcacccacgtggcccag

SEQ ID NO: 539 - DNA, 45 bp, synthetic construct
  misc_feature 1..45: note = Description of Artificial Sequence: Synthetic oligonucleotide
  source 1..45: mol_type = other DNA
aagaggatctggttcatccccaggagcagctggtacgagagggcc

SEQ ID NO: 540 - DNA, 45 bp, synthetic construct
  misc_feature 1..45: note = Description of Artificial Sequence: Synthetic oligonucleotide
  source 1..45: mol_type = other DNA
atgcccctgagcaggtactggtggctgttcagccacaggcccagg

SEQ ID NO: 541 - DNA, 45 bp, synthetic construct
  misc_feature 1..45: note = Description of Artificial Sequence: Synthetic oligonucleotide
  source 1..45: mol_type = other DNA
aggcacctgagccacttcaagtggctgaggagccacggcctggac

SEQ ID NO: 542 - DNA, 45 bp, synthetic construct
  misc_feature 1..45: note = Description of Artificial Sequence: Synthetic oligonucleotide
  source 1..45: mol_type = other DNA
aggaggttccacttccacagcaggatggtggccgtggacaacagc

SEQ ID NO: 543 - DNA, 48 bp, synthetic construct; identical to SEQ ID NO: 268
  misc_feature 1..48: note = Description of Artificial Sequence: Synthetic oligonucleotide
  source 1..48: mol_type = other DNA

SEQ ID NO: 544 - DNA, 45 bp, synthetic construct; identical to SEQ ID NO: 269
  misc_feature 1..45: note = Description of Artificial Sequence: Synthetic oligonucleotide
  source 1..45: mol_type = other DNA

SEQ ID NO: 545 - DNA, 48 bp, synthetic construct; identical to SEQ ID NO: 270
  misc_feature 1..48: note = Description of Artificial Sequence: Synthetic oligonucleotide
  source 1..48: mol_type = other DNA

SEQ ID NO: 546 - DNA, 39 bp, synthetic construct; identical to SEQ ID NO: 271
  misc_feature 1..39: note = Description of Artificial Sequence: Synthetic oligonucleotide
  source 1..39: mol_type = other DNA

SEQ ID NO: 547 - DNA, 42 bp, synthetic construct; identical to SEQ ID NO: 272
  misc_feature 1..42: note = Description of Artificial Sequence: Synthetic oligonucleotide
  source 1..42: mol_type = other DNA

SEQ ID NO: 548 - protein, 98 aa, synthetic construct
  REGION 1..98: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 549 - protein, 12 aa, synthetic construct
  REGION 1..12: note = Description of Artificial Sequence: Synthetic peptide
EPKSCDKTHTCP

SEQ ID NO: 550 - protein, 113 aa, synthetic construct
  REGION 1..113: note = Description of Artificial Sequence: Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 551 - protein, 107 aa, synthetic construct
  REGION 1..107: note = Description of Artificial Sequence: Synthetic polypeptide
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 552 - protein, 98 aa, synthetic construct
  REGION 1..98: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 553 - protein, 107 aa, synthetic construct
  REGION 1..107: note = Description of Artificial Sequence: Synthetic polypeptide
GQPREPQVYTLPPSREELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 554 - protein, 107 aa, synthetic construct
  REGION 1..107: note = Description of Artificial Sequence: Synthetic polypeptide
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 555 - protein, 30 aa, synthetic construct
  REGION 1..30: note = Description of Artificial Sequence: Synthetic polypeptide
  VARIANT 1..30: note = This sequence may encompass 2-6 "Gly Gly Gly Gly Ser" repeating units
GGGGSGGGGSGGGGSGGGGSGGGGSGGGGS

SEQ ID NO: 556 - protein, 75 aa, synthetic construct
  REGION 1..75: note = Description of Artificial Sequence: Synthetic polypeptide
  VARIANT 1..75: note = This sequence may encompass 0-15 "Gly Gly Gly Gly Ser" repeating units
GGGGSGGGGSGGGGSGGGGSGGGGSGGGGSGGGGSGGGGSGGGGSGGGGSGGGGSGGGGSGGGGSGGGGSGGGGS

SEQ ID NO: 557 - protein, 30 aa, synthetic construct
  REGION 1..30: note = Description of Artificial Sequence: Synthetic polypeptide
  VARIANT 1..30: note = This sequence may encompass 0-15 "Gly Ser" repeating units
GSGSGSGSGSGSGSGSGSGSGSGSGSGSGS

SEQ ID NO: 558 - protein, 30 aa, synthetic construct
  REGION 1..30: note = Description of Artificial Sequence: Synthetic polypeptide
  VARIANT 1..30: note = This sequence may encompass 0-15 "Ser Gly" repeating units
SGSGSGSGSGSGSGSGSGSGSGSGSGSGSG

SEQ ID NO: 559 - protein, 45 aa, synthetic construct
  REGION 1..45: note = Description of Artificial Sequence: Synthetic polypeptide
  VARIANT 1..45: note = This sequence may encompass 0-15 "Gly Ser Gly" repeating units
GSGGSGGSGGSGGSGGSGGSGGSGGSGGSGGSGGSGGSGGSGGSG

SEQ ID NO: 560 - protein, 60 aa, synthetic construct
  REGION 1..60: note = Description of Artificial Sequence: Synthetic polypeptide
  VARIANT 1..60: note = This sequence may encompass 0-15 "Ser Gly Ser Gly" repeating units
SGSGSGSGSGSGSGSGSGSGSGSGSGSGSGSGSGSGSGSGSGSGSGSGSGSGSGSGSGSG

SEQ ID NO: 561 - protein, 12 aa, synthetic construct
  REGION 1..12: note = Description of Artificial Sequence: Synthetic peptide
ESKYGPPCPPCP

SEQ ID NO: 562 - protein, 6 aa, synthetic construct
  REGION 1..6: note = Description of Artificial Sequence: Synthetic 6xHis tag
HHHHHH

SEQ ID NO: 563 - protein, 10 aa, synthetic construct
  REGION 1..10: note = Description of Artificial Sequence: Synthetic peptide
GGGGSGGGGS

SEQ ID NO: 564 - protein, 22 aa, synthetic construct
  REGION 1..22: note = Description of Artificial Sequence: Synthetic peptide
VVGVVGGLLGSLVLLVWVLAVI